(19) United States
(12) Patent Application Publication
(10) Pub. No.: US 2009/0265114 A1
(43) Pub. Date: Oct. 22, 2009

(54) Influenza virus neuraminidase crystal structure and their use thereof

(76) Inventors: Steven John Gamblin, London (GB); Alan James Hay, London (GB); John James Skehel, London (GB); Lesley Findlay Haire, London (GB); David John Stevens, London (GB); Rupert Russell, St. Andrews (GB); Patrick James Collins, London (GB); Yi Pu Lin, London (GB); George Michael Blackburn, Sheffield (GB)

Correspondence Address: NIXON & VANDERHYE, PC, 901 NORTH GLEBE ROAD, 11TH FLOOR, ARLINGTON, VA, 22203, US

(21) Appl. No.: 12/308,065

(22) PCT Filed: Jun. 6, 2007
(86) PCT No.: PCT/GB2007/002065

§ 371 Date: Apr. 7, 2009

(30) Foreign Application Priority Data
Jun. 6, 2006 (GB) 0611136.3

(51) Int. Cl. G06F 19/00; C12N 9/24; C07K 14/11

(52) U.S. Cl. 702/19; 435/200; 702/27; 530/350

(57) ABSTRACT

The invention relates to crystals of the influenza virus neuraminidase protein, their structures and their use.

### FIELD OF THE INVENTION

The present invention relates to the crystals of the influenza virus neuraminidase protein, their structures and their use.

### BACKGROUND TO THE INVENTION

### Influenza Virus

Influenza A virus is an RNA virus which can vary in virulence. The two major surface glycoproteins of Influenza viruses are haemagglutinin (HA) and neuraminidase (NA). HA mediates cell-surface sialic acid receptor binding to initiate virus infection. Following virus replication, NA removes sialic acid from virus and cellular glycoproteins to facilitate virus release and the spread of infection to new cells. The distinct antigenic properties of different HA and NA molecules are used to classify influenza type A viruses into subtypes: 16 for HA (H1-H16) and 9 for NA (N1-N9). Different combinations of HA and NA subtypes are found in avian species and, in humans, the three pandemics of the twentieth century were caused by viruses containing H1N1 in 1918, H2N2 in 1957 and H3N2 in 1968. The N1 and N2 NAs belong to phylogenetically distinct groups. Group-1 contains N1, N4, N5 and N8 subtypes while Group-2 contains N2, N3, N6, N7 and N9.

NA has been targeted in structure-based enzyme inhibitor design programmes that have resulted in the production of two drugs, Relenza (zanamivir) and Tamiflu (oseltamivir). The success of these developments has been attributed in part to proposals that the catalytic sites of the enzymes are an invariant feature which might be exploited for sub-type independent therapy and to observations that they are comparatively rigid with only minor conformational changes in the sites on inhibitor binding. The X-ray crystallographic structural information that supports these conclusions is only available for the Group-2 NAs, N2 and N9, but the idea that the active sites of the Group-1 enzymes would be similar was supported by observations that the structures of more distantly related influenza type B NAs are similar to those of the Group-2 enzymes. Nevertheless, different drug-resistant NA mutant viruses have arisen following Tamiflu treatment of humans infected with viruses containing different NA subtypes. There are also indications that inhibitor structure/activity relationships do not apply across subtypes.

A highly pathogenic form of avian influenza virus, H5N1, appeared in the Far East towards the end of the twentieth century and its continued spread worldwide has raised fears that this virus may acquire the genetic changes necessary for it to transmit effectively from human to human, initiating a new pandemic. Before effective vaccines might be developed the chief hope of moderating the impact of such an outbreak rests with inhibitors of NA.

### DISCLOSURE OF THE INVENTION

The present invention relates to crystals and the crystal structures of three N1 group NAs, namely N1, N4 and N8. It has been found that while the active sites of the three proteins are substantially similar to each other, there are significant conformational differences between these active sites and those of members of the N2 group of NAs. These differences bring about changes to the biding pocket of the N1 group of neuraminidase enzymes which would not be apparent from homology modelling (or using similar techniques) based on currently available Group-2 NA structures.

More particularly, the present inventors have obtained apo crystals of N1, N4 and N8, as well as co-crystals of these proteins with one or more NA inhibitors. Thus in one aspect, the invention provides a three dimensional structure of an N1 group neuraminidase as set out in any one of Tables 1 to 4, and uses, described further herein below of the three dimensional structure of an N1 group neuraminidase set out in any one of Tables 1 to 3.

Reference herein to the structures of Tables 1 to 3, or the structures of any one of Tables 1 to 3 thus includes the individual Tables 1, 2 and 3.

In general aspects, the present invention is concerned with the provision of an N1 group neuraminidase structure and its use in modelling the interaction of molecular structures, e.g. potential and existing pharmaceutical compounds, (including prodrugs, inhibitors or substrates), or fragments of such compounds, with this structure.

These and other aspects and embodiments of the present invention are discussed below.

### Brief Description of the Tables

Table 1 (FIG. 1) sets out the coordinate data of the structure of neuraminidase N1.

Table 2 (FIG. 2) sets out the coordinate data of the structure of neuraminidase N4.

Table 3 (FIG. 3) sets out the coordinate data of the structure of neuraminidase N8.

### BRIEF DESCRIPTION OF THE DRAWINGS

FIG. 1 sets out Table 1.

FIG. 2 sets out Table 2.

FIG. 3 sets out Table 3.

FIG. 4 is an alignment of the N1, N4 and N8 proteins of FIGS. 1-3, numbered according to a consensus numbering system used in Tables 1-3. The consensus numbering includes insertions after residues 169, 330, 342 and 412 designated A, B, etc such that consecutive numbering (170, 331, 343, 413) continues after the insertion. Resides whose consensus number is a multiple of 10 are indicated by an asterisk above the alignment. Reference herein to amino acid numbers are to the numbering of the alignment and not to the numbers of individual sequences SEQ ID NO:1, 2 and 3 unless explicitly indicated to the contrary.

FIG. 5 shows a superposition of the active site of N1 (dark grey) and N9 (light grey) NAs. Residues such as Glu-276, Glu-119, Asp-151 and the hydrophobic residue at position 149 that adopt different conformations between Group-1 and Group-2 NAs are shown in stick representation.

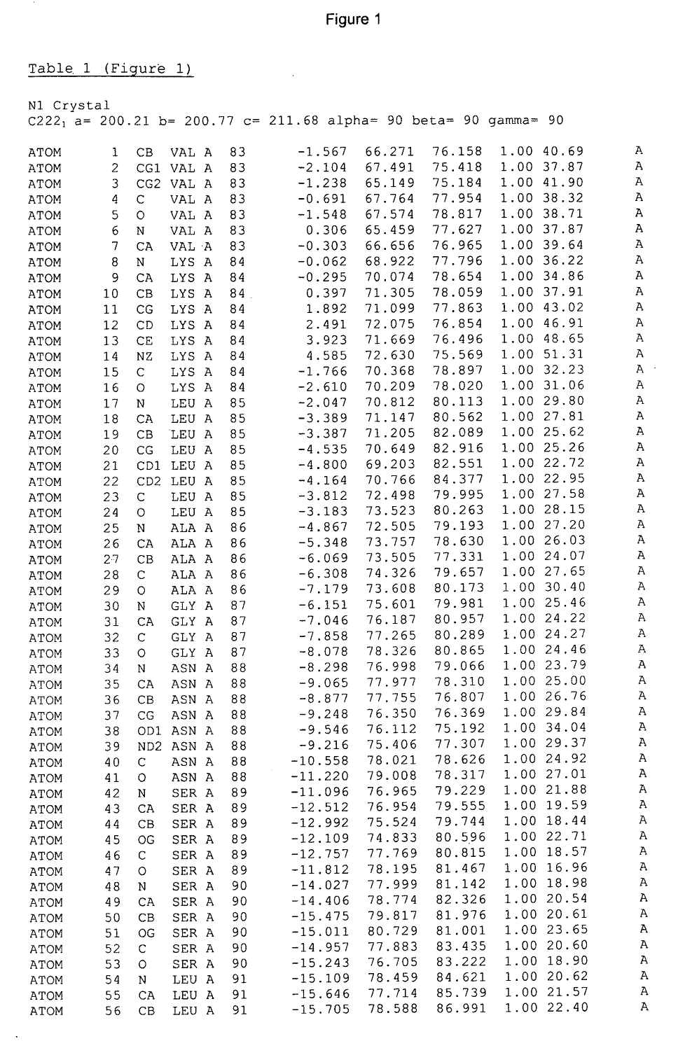

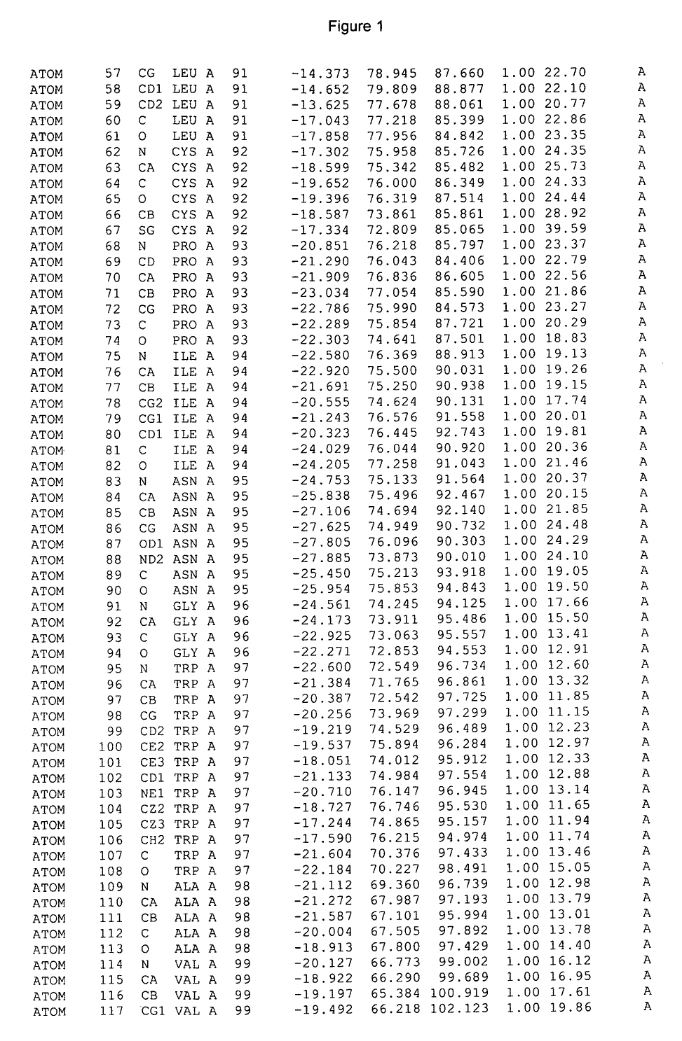

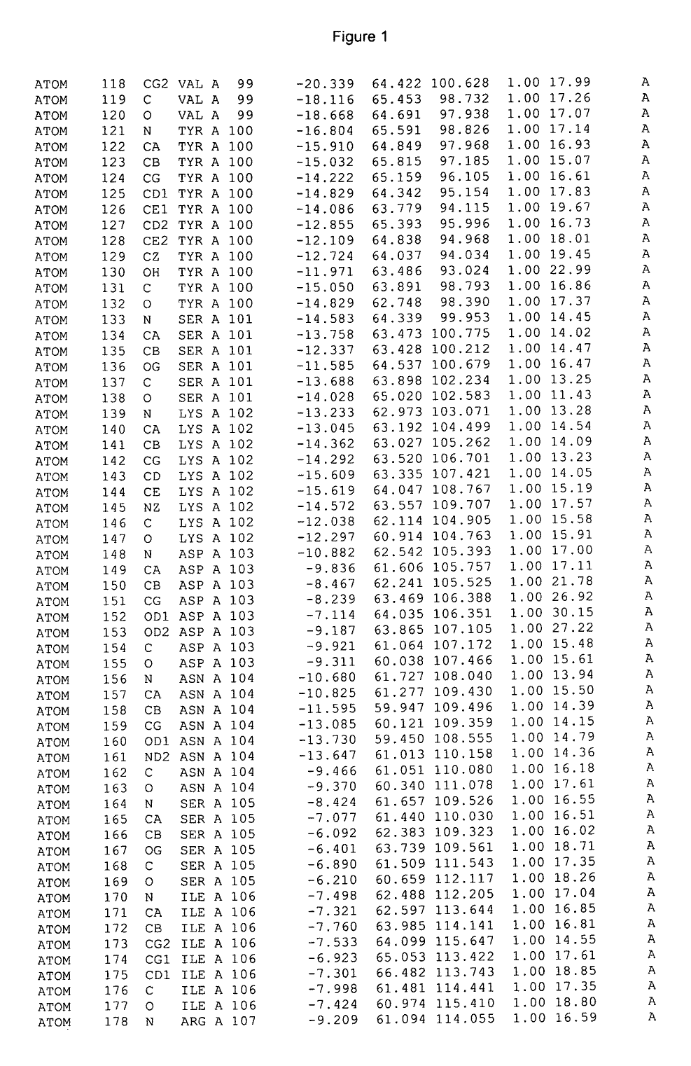

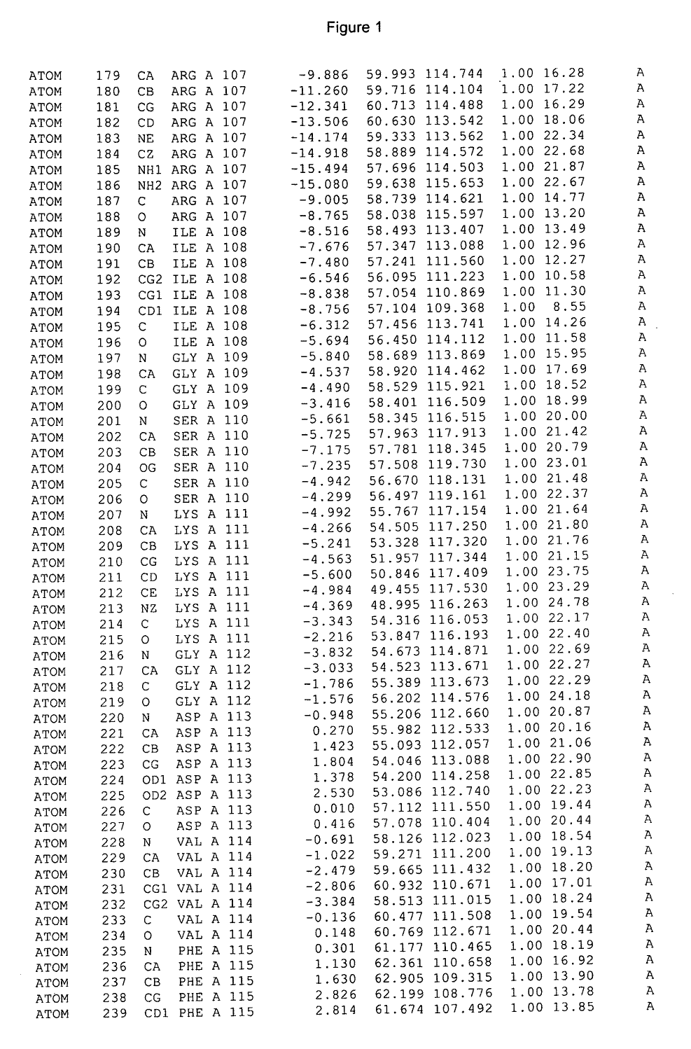

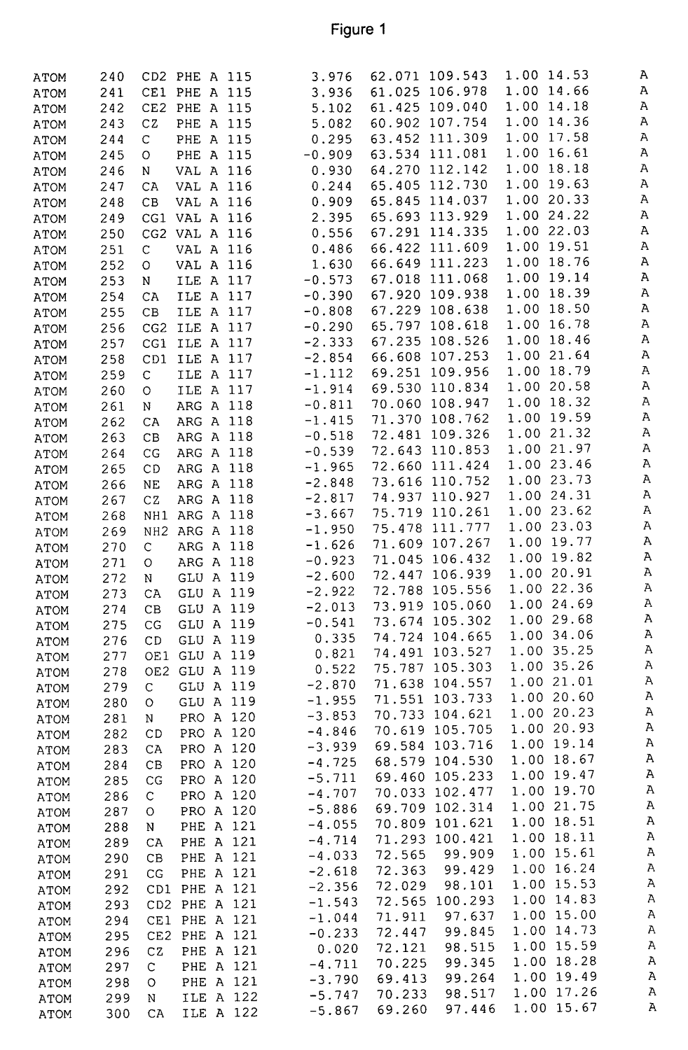

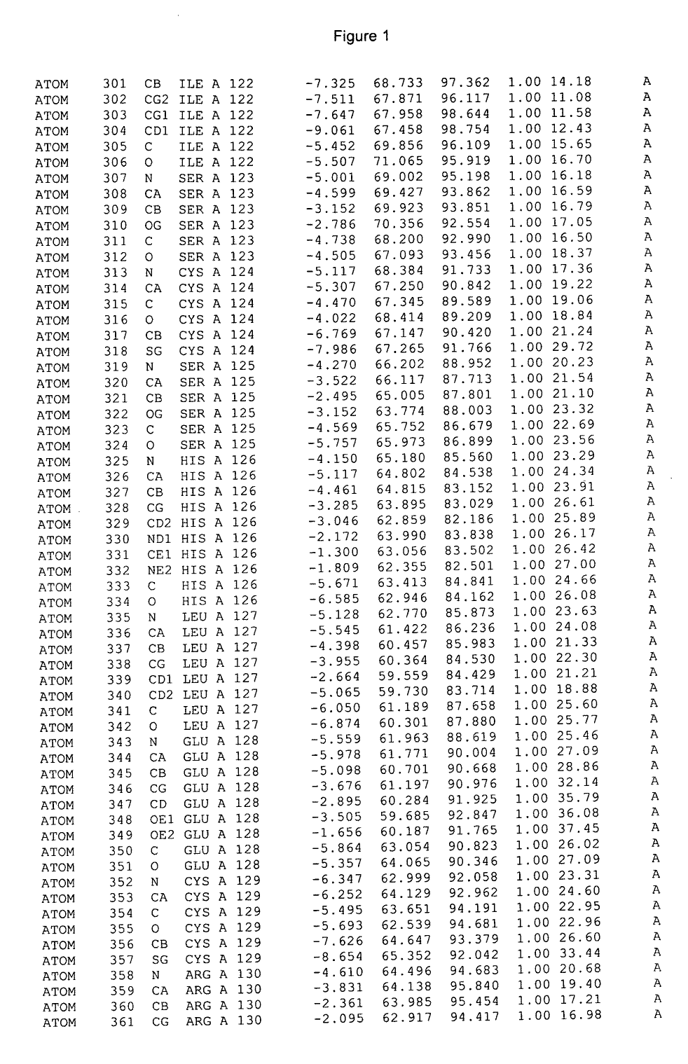

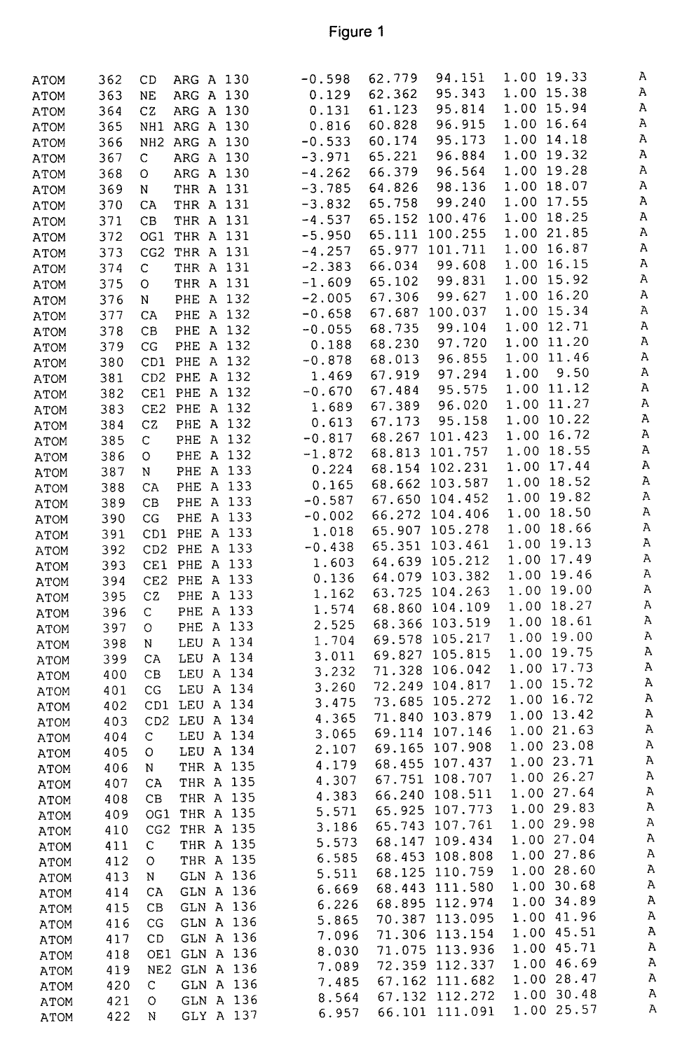

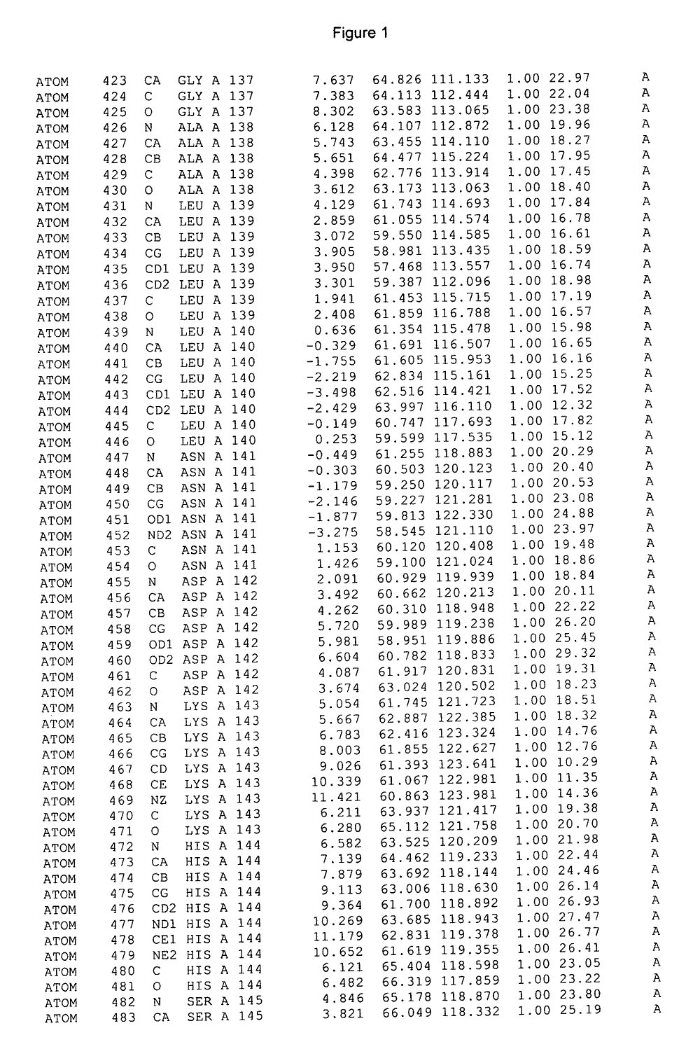

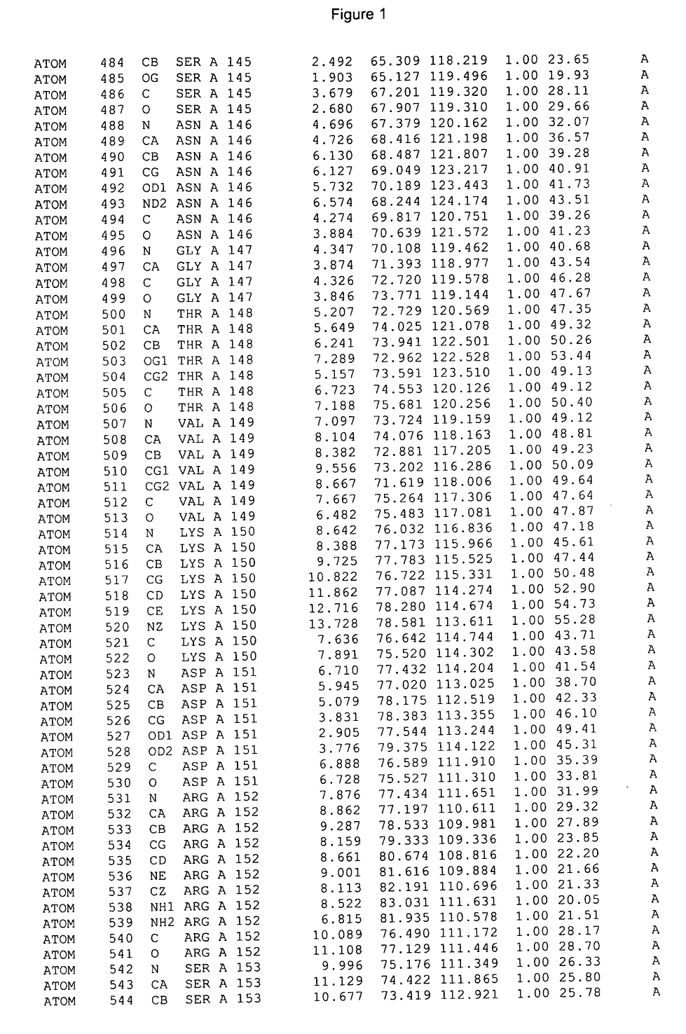

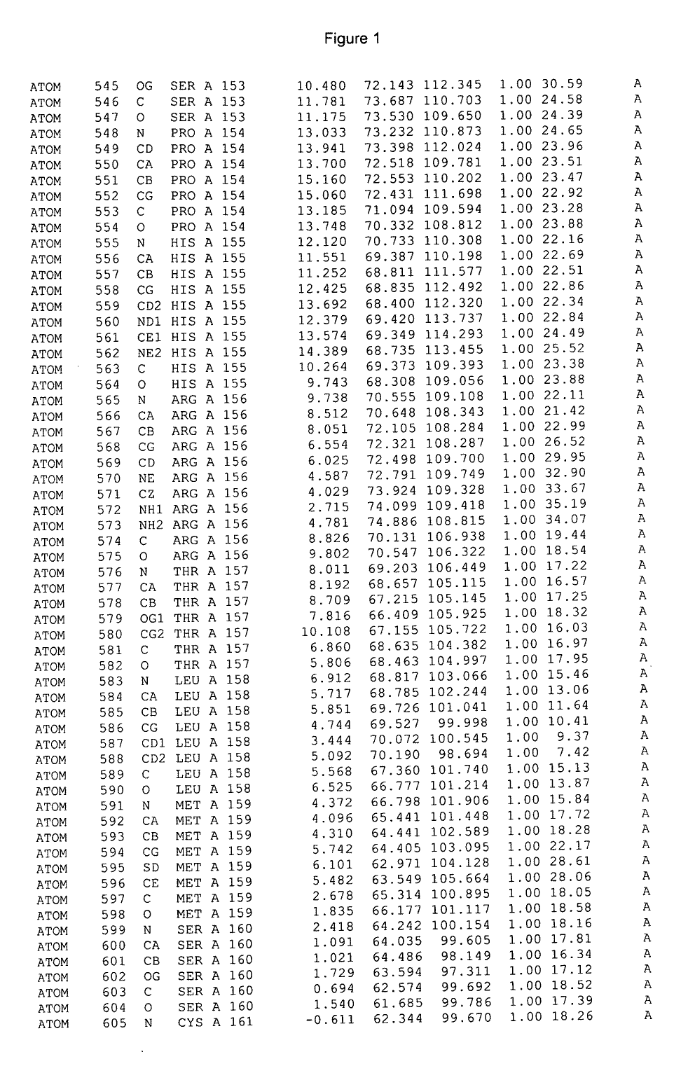

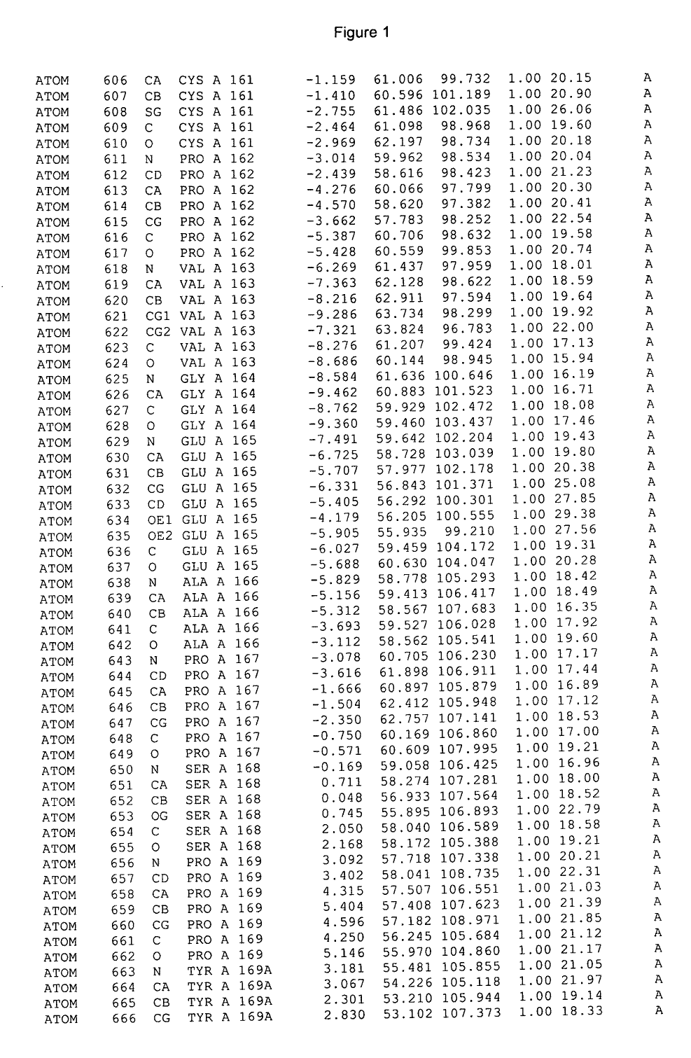

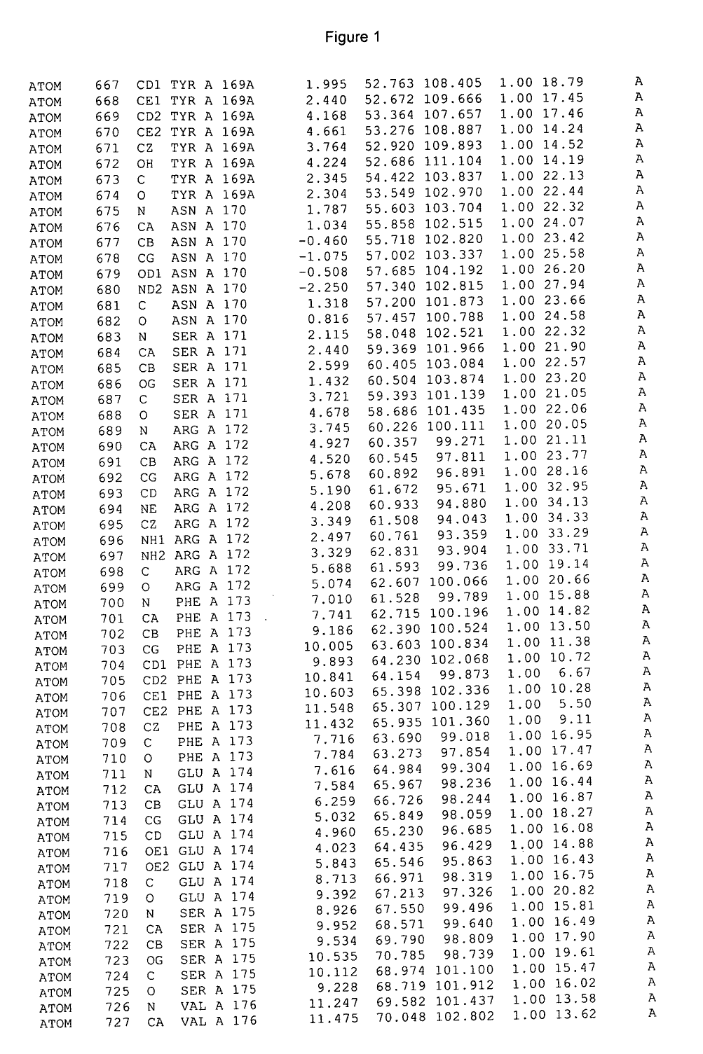

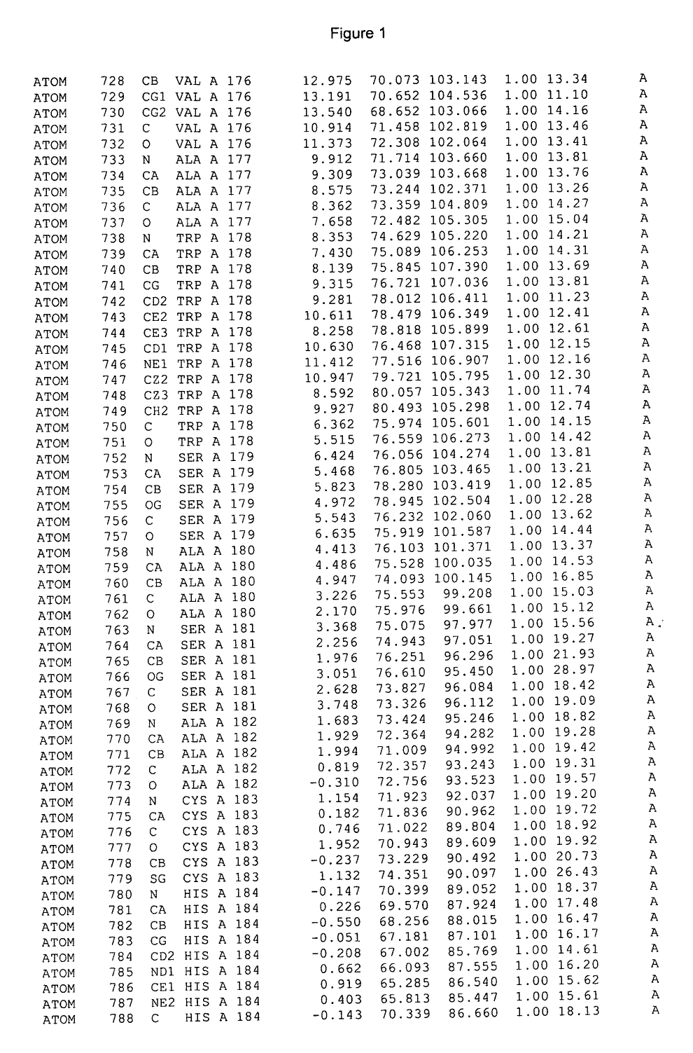

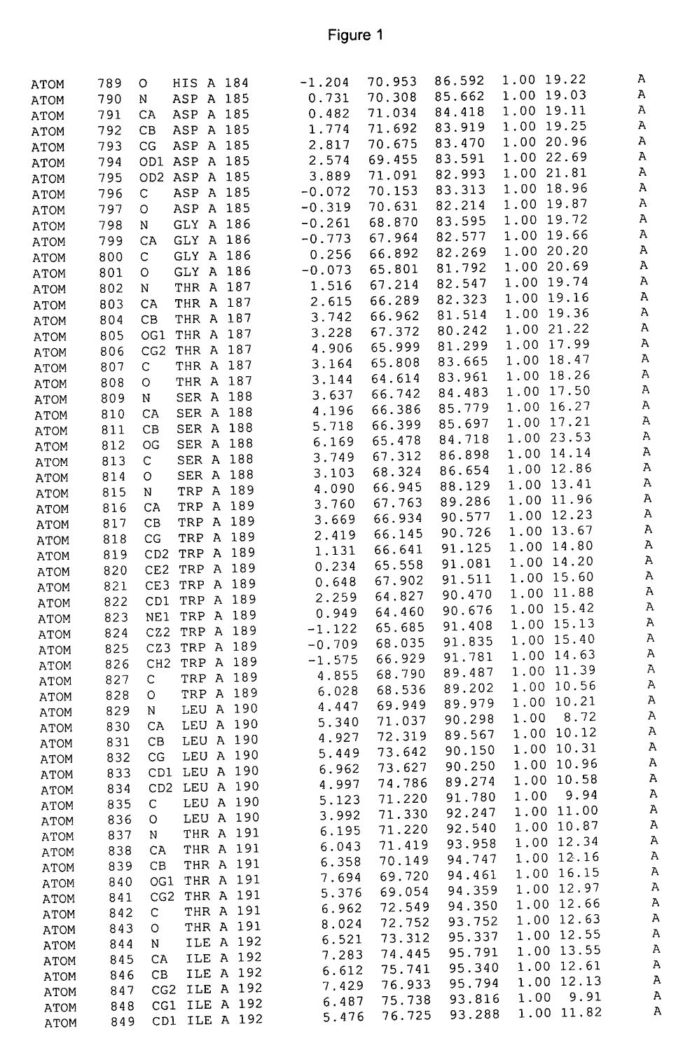

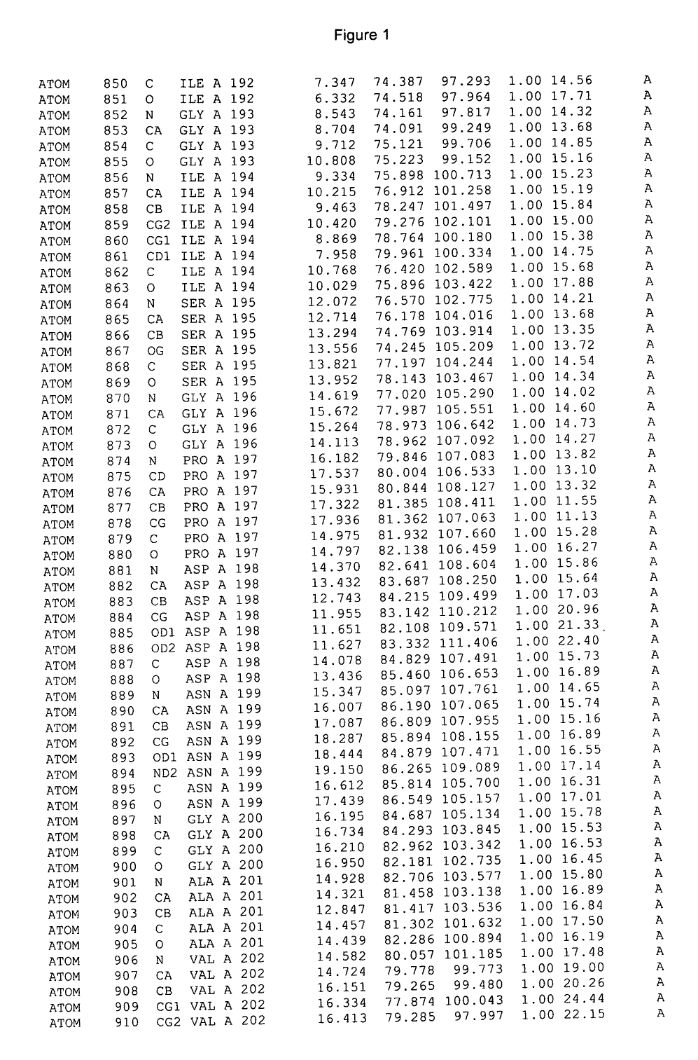

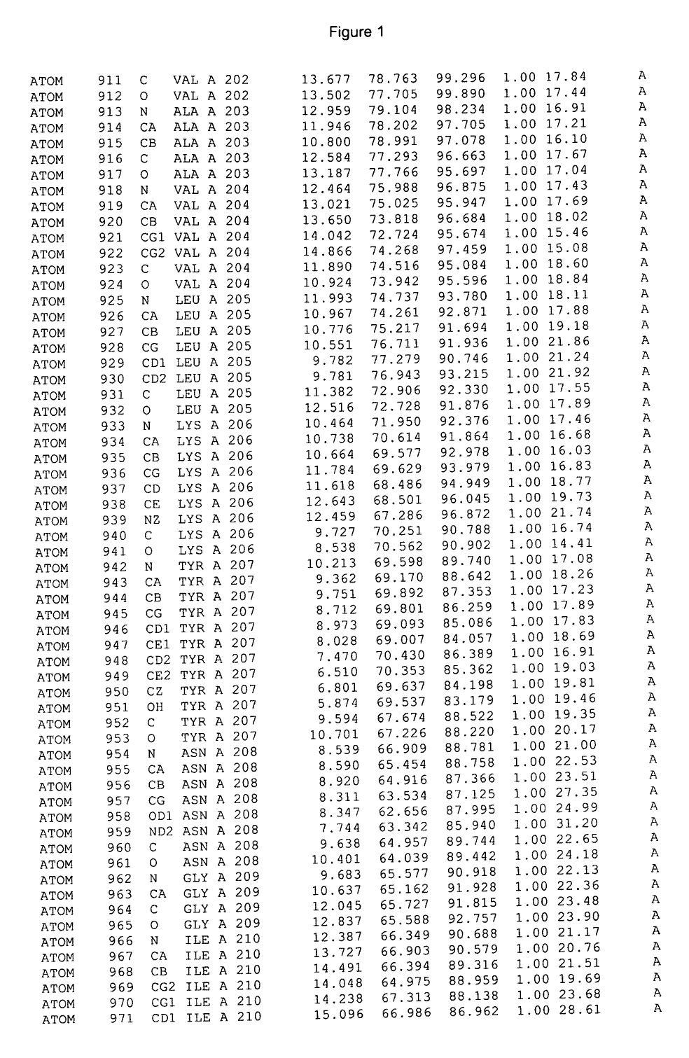

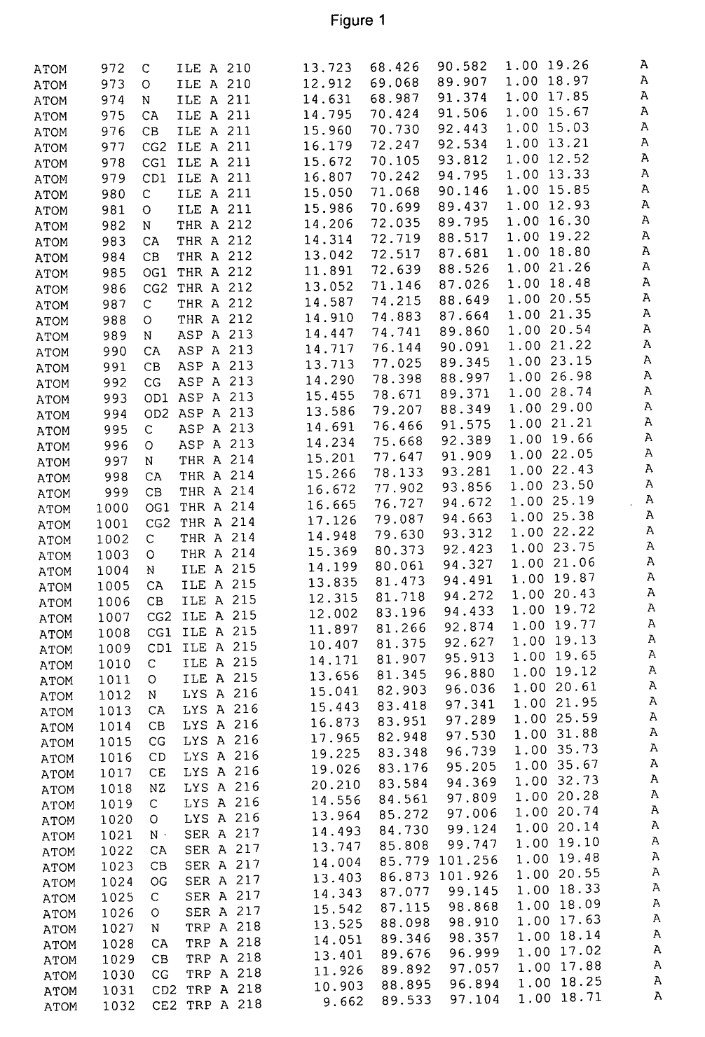

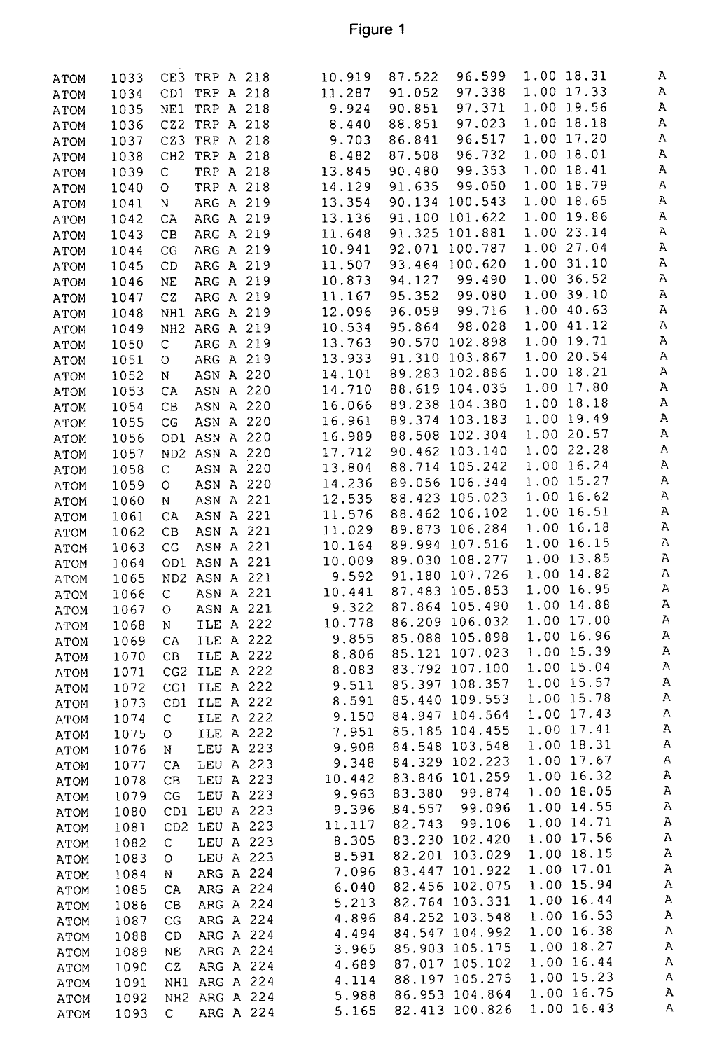

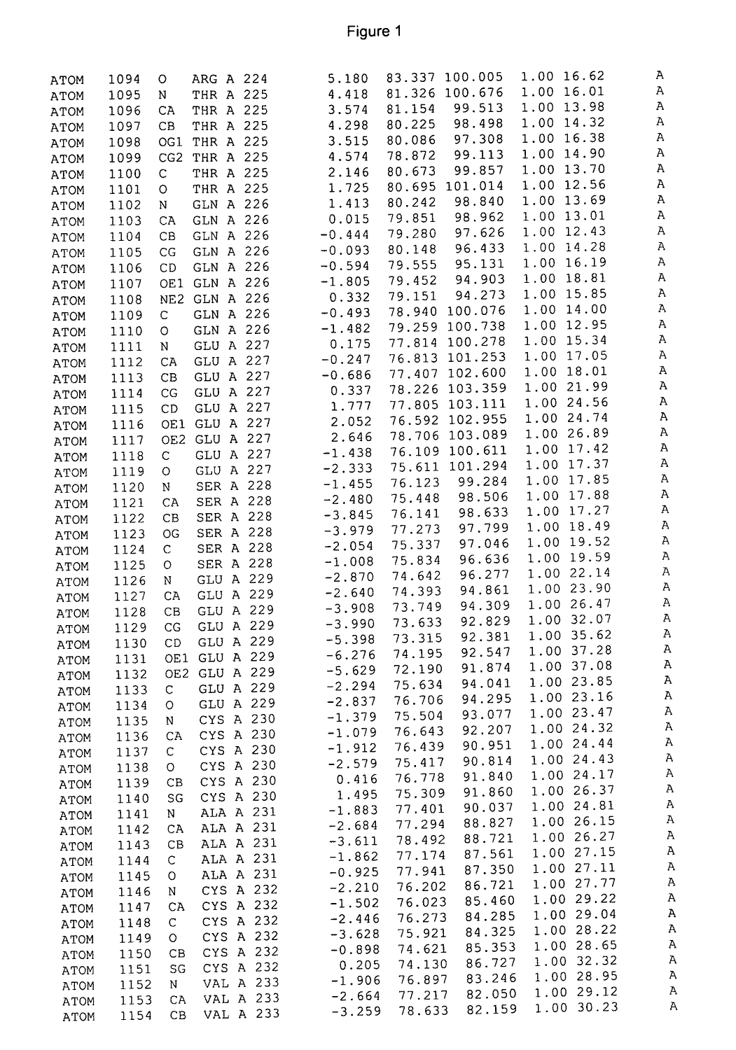

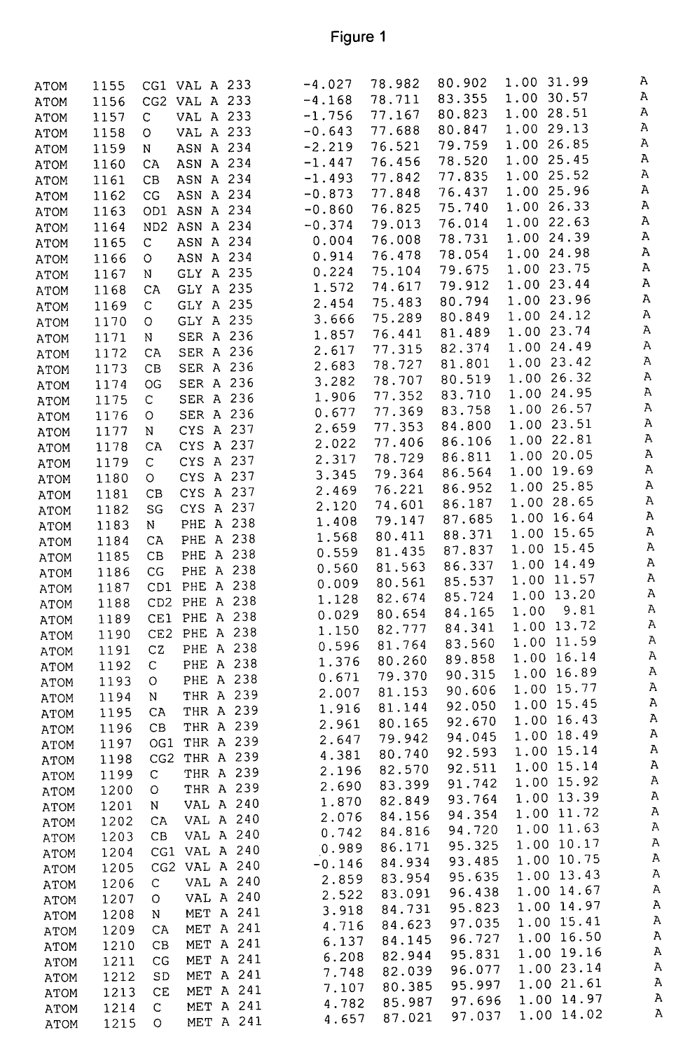

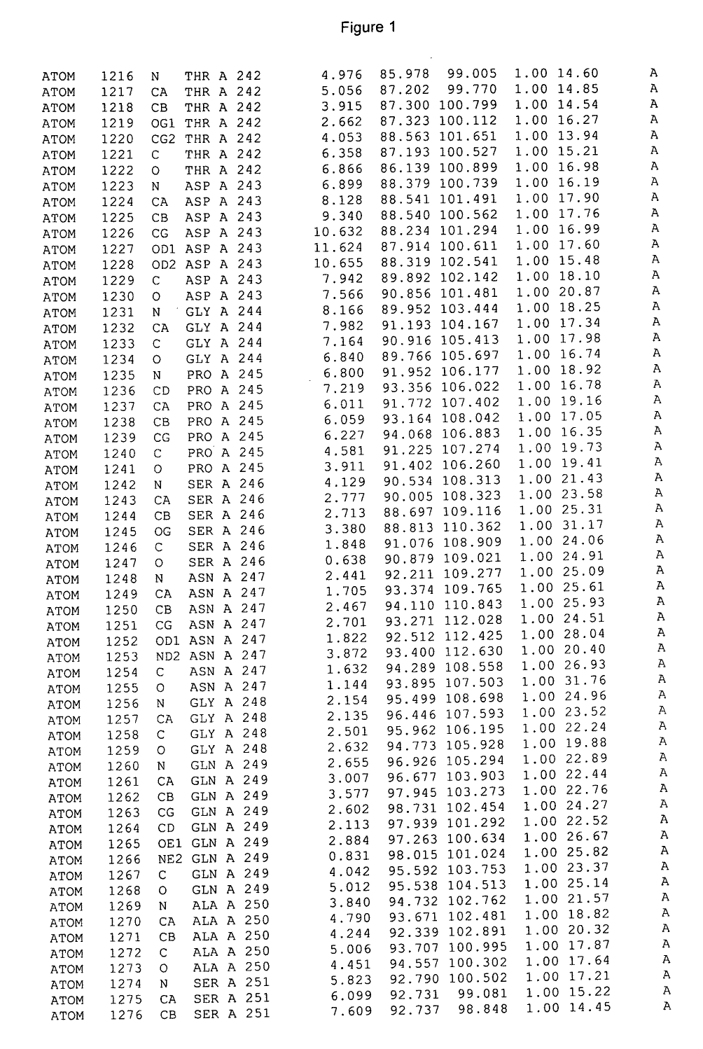

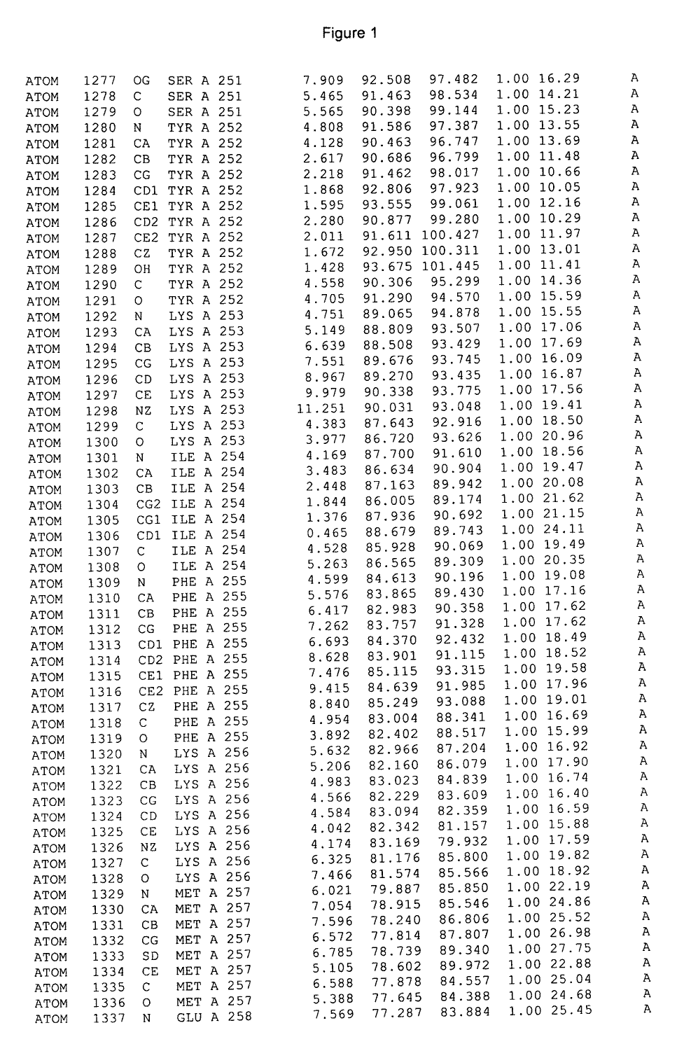

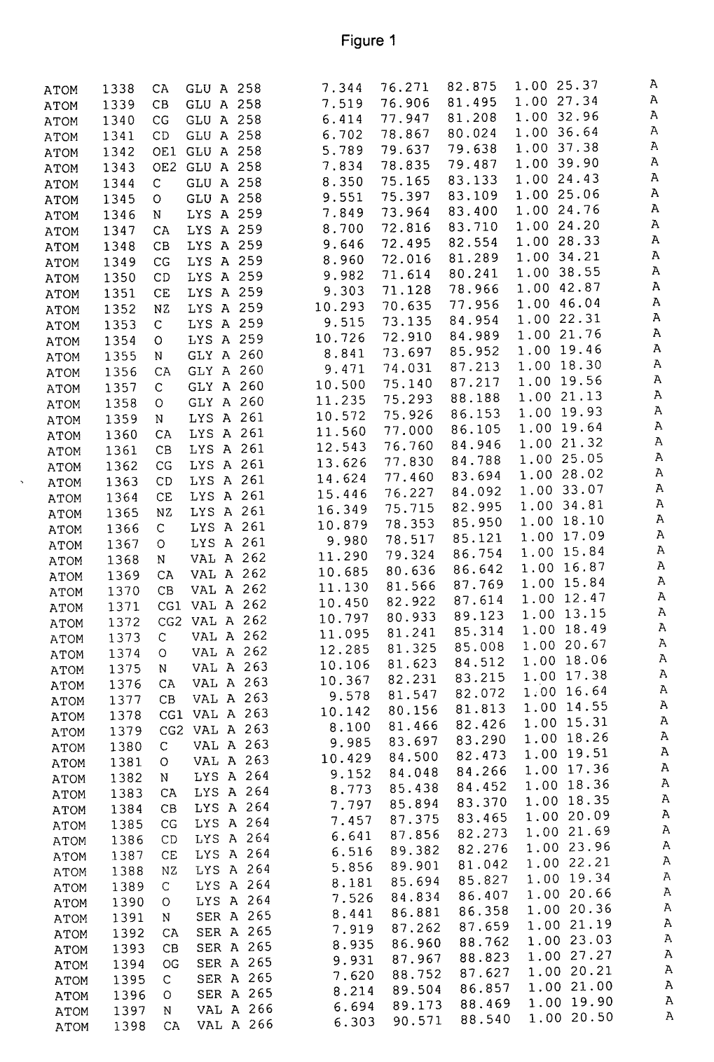

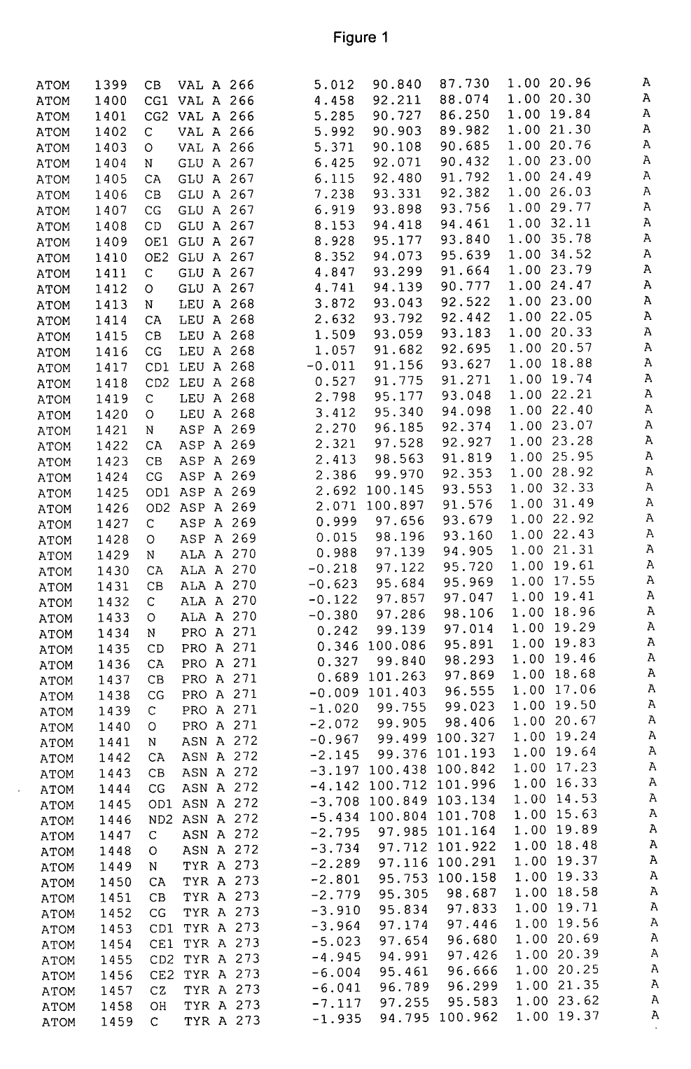

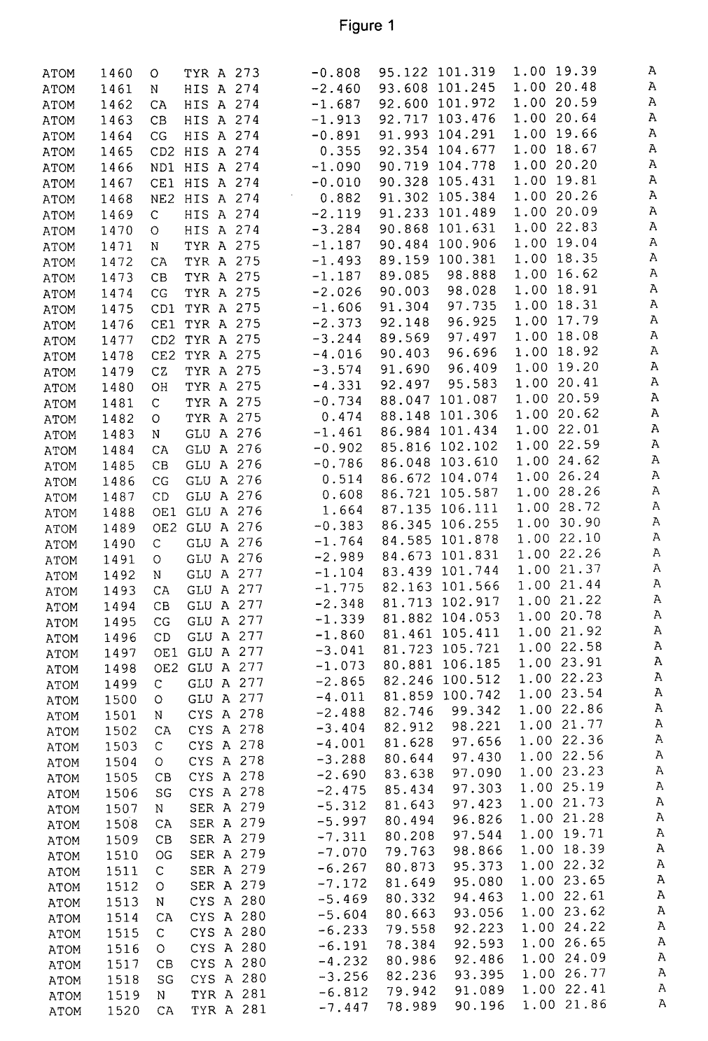

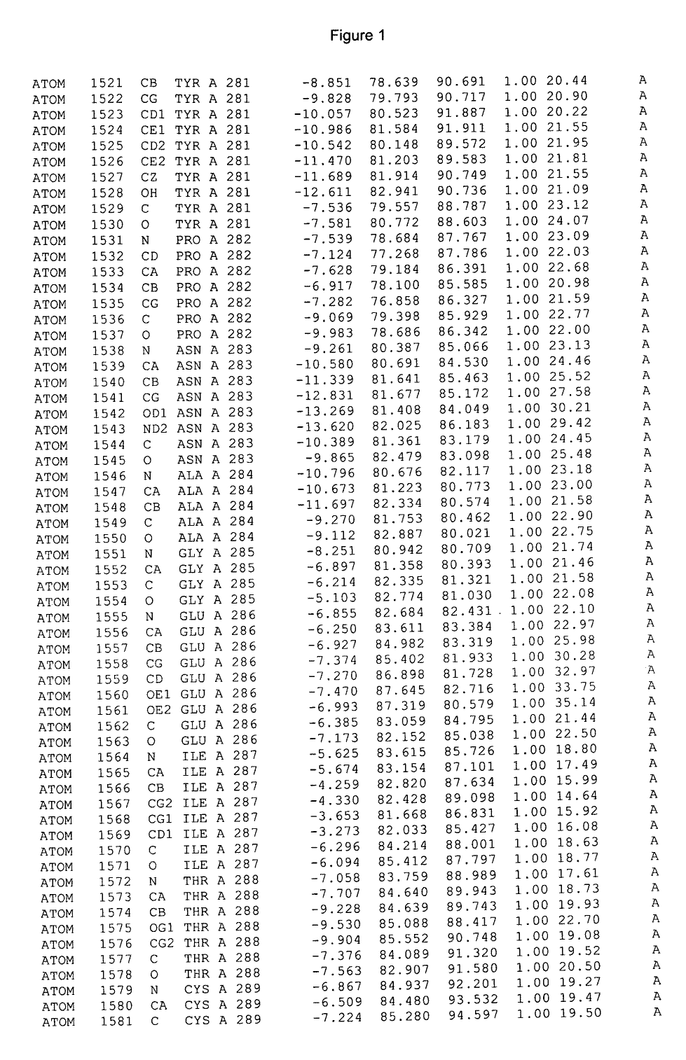

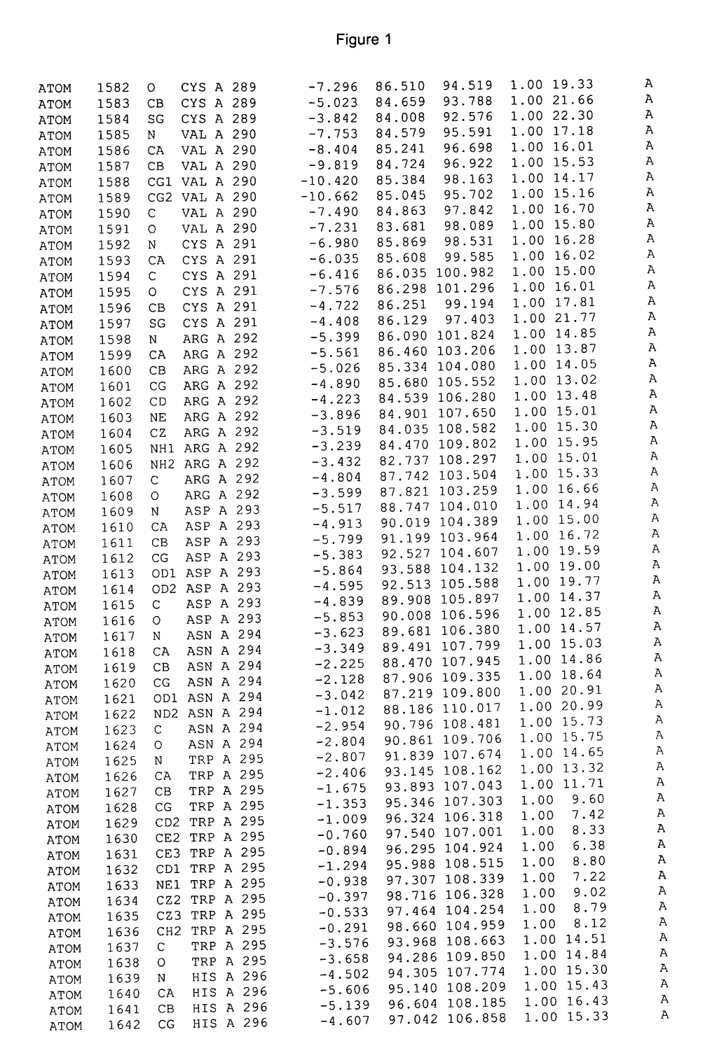

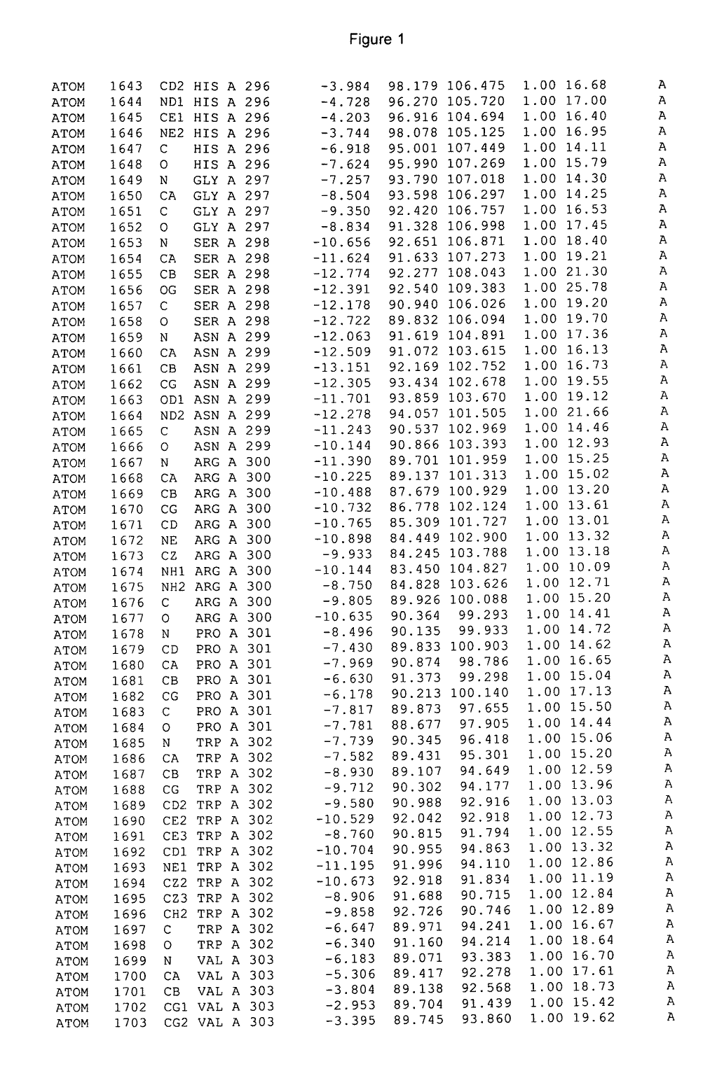

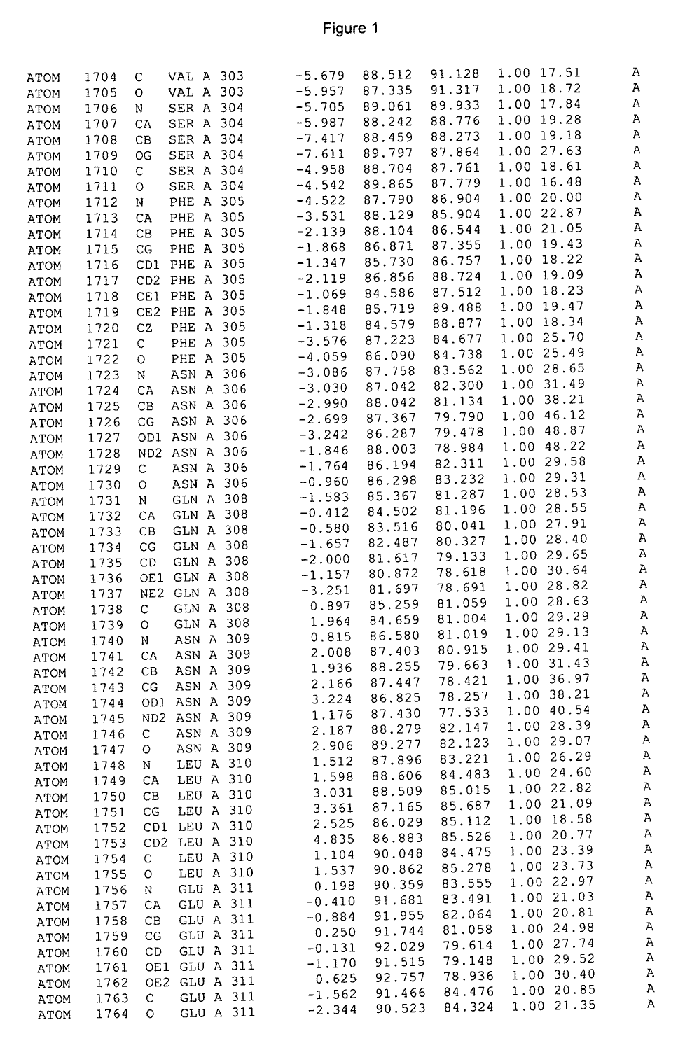

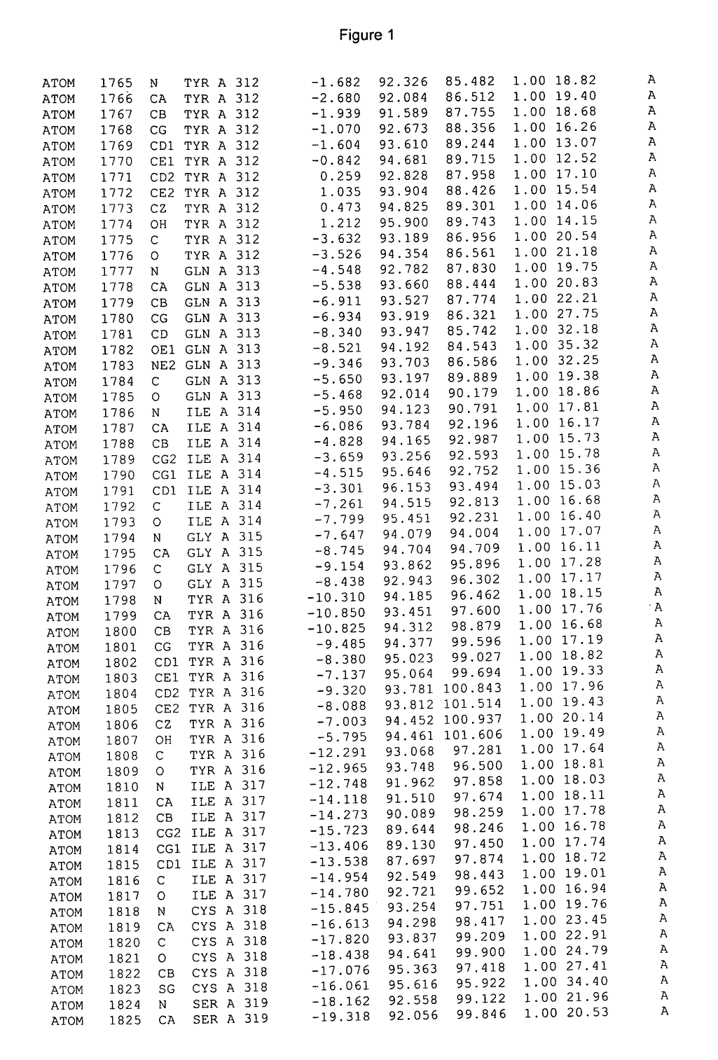

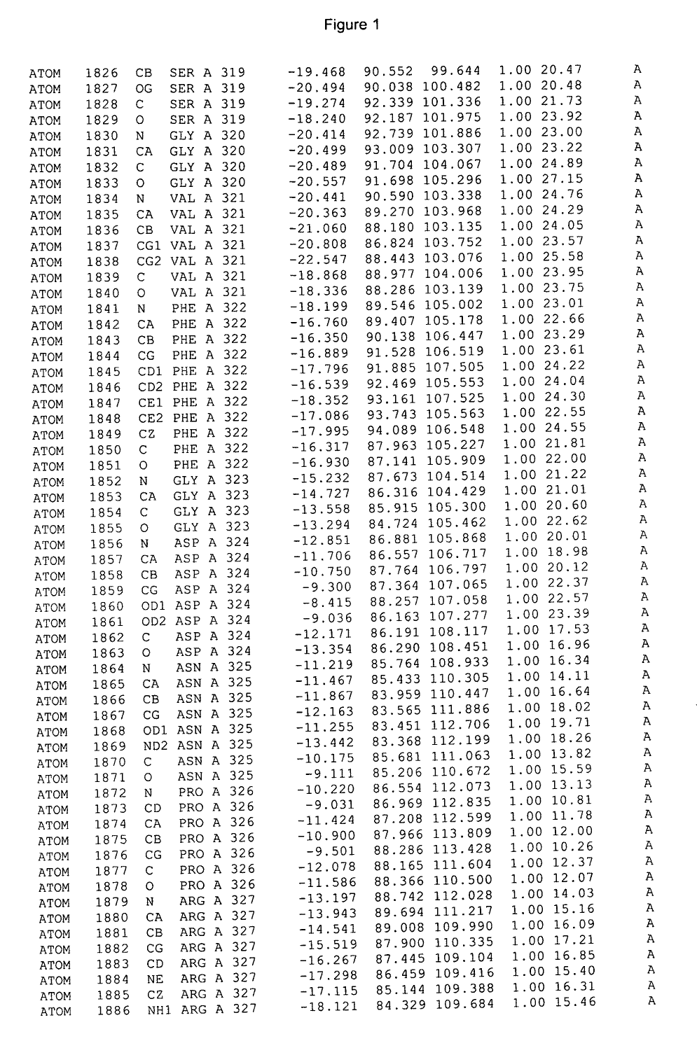

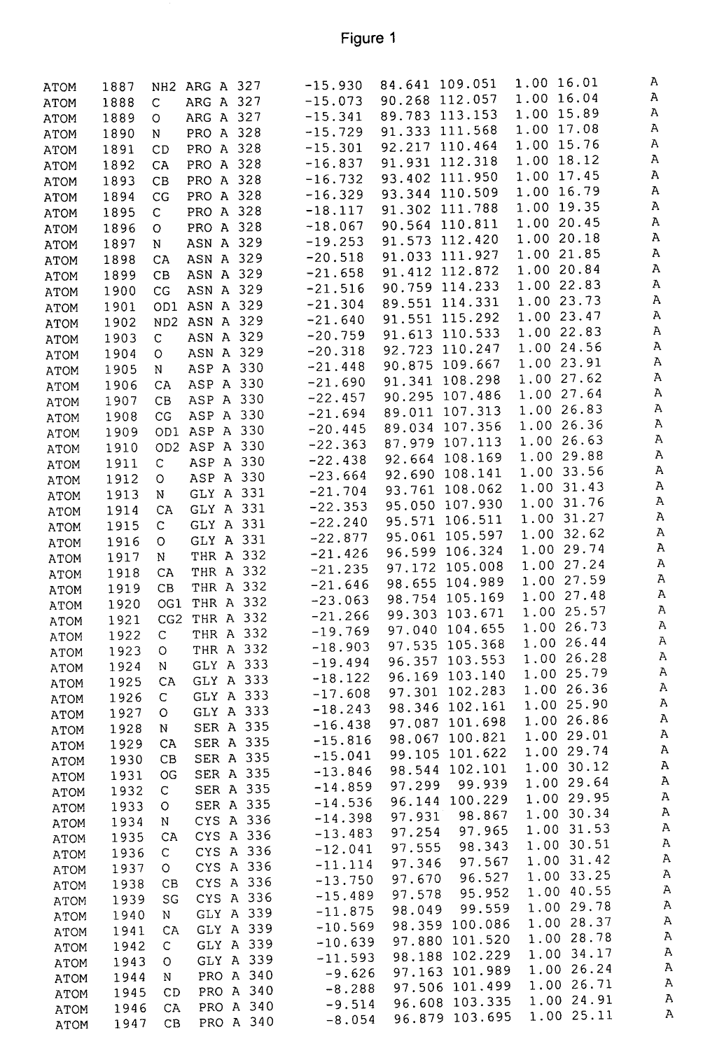

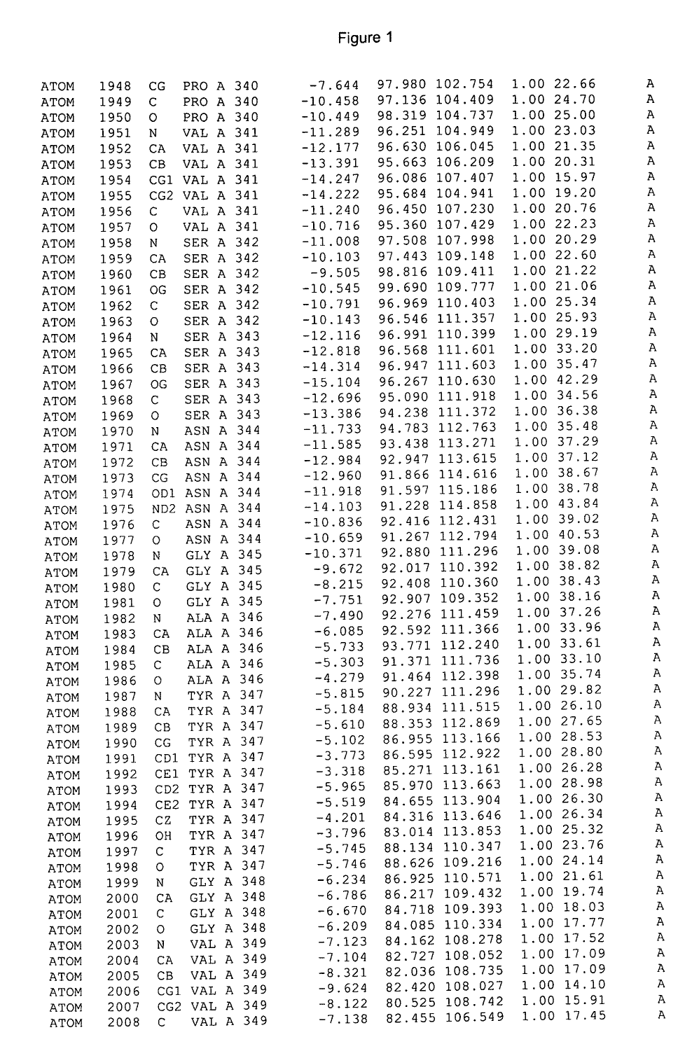

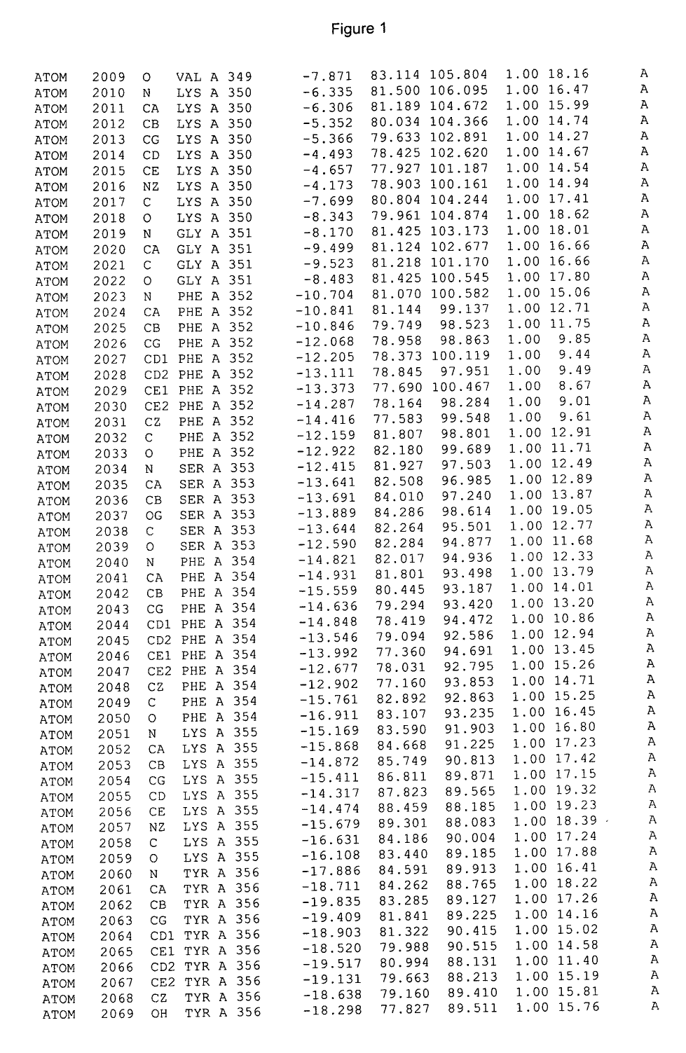

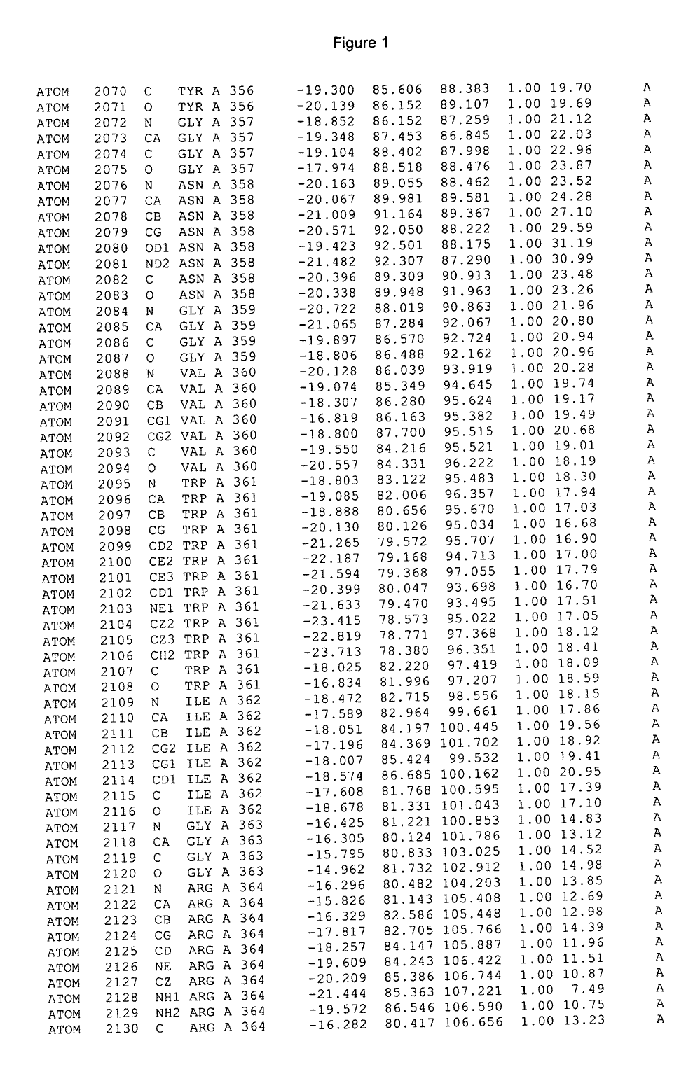

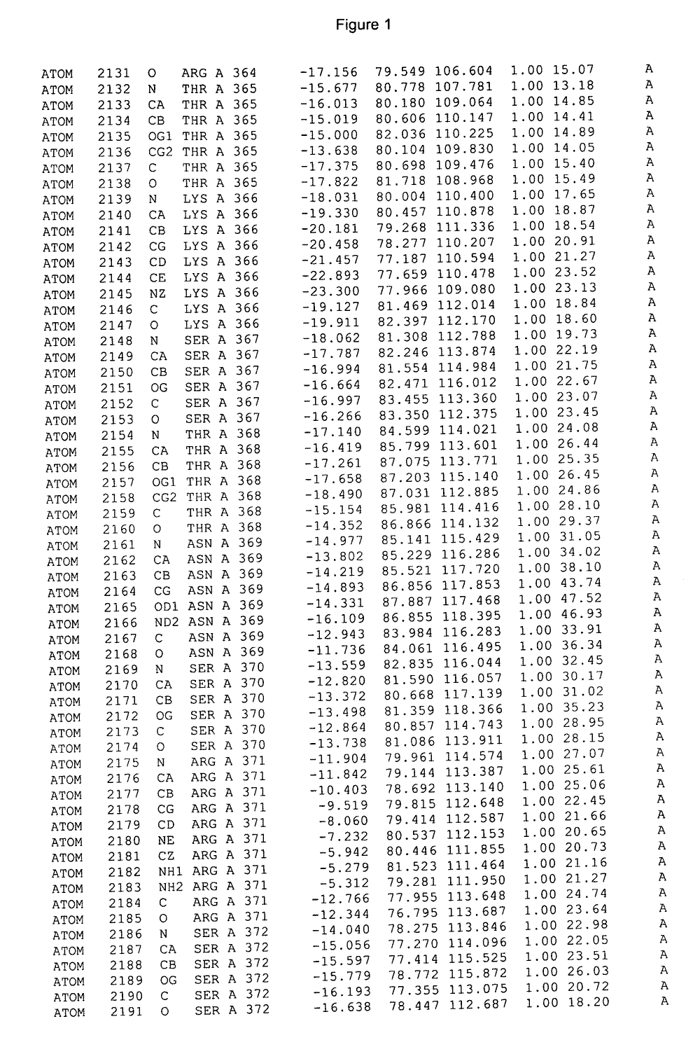

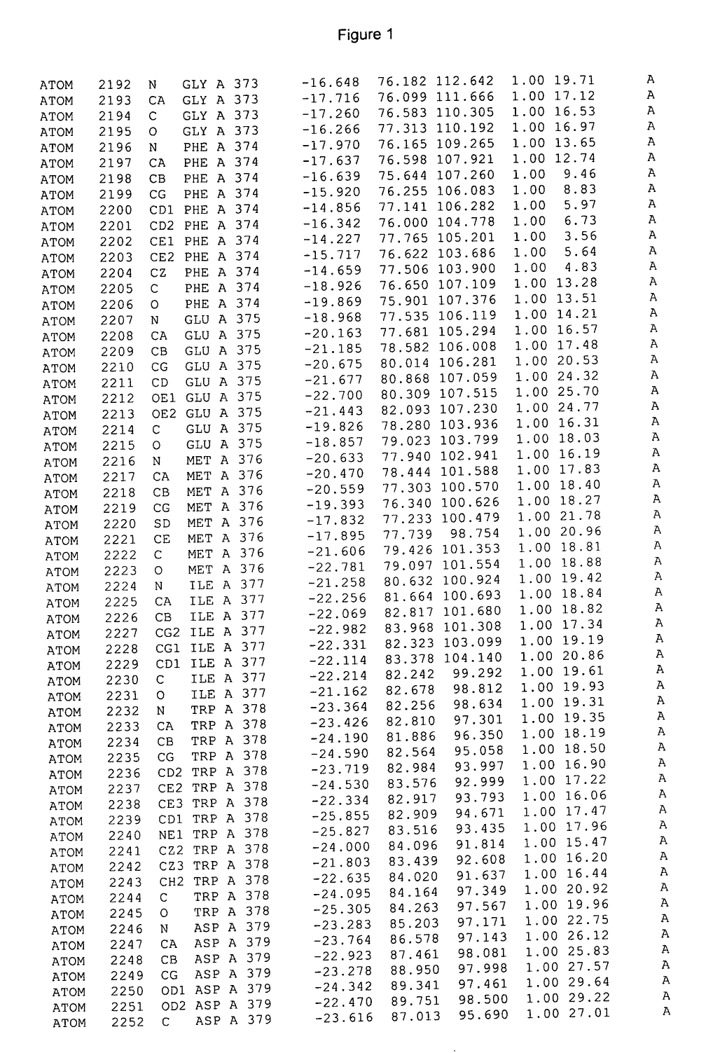

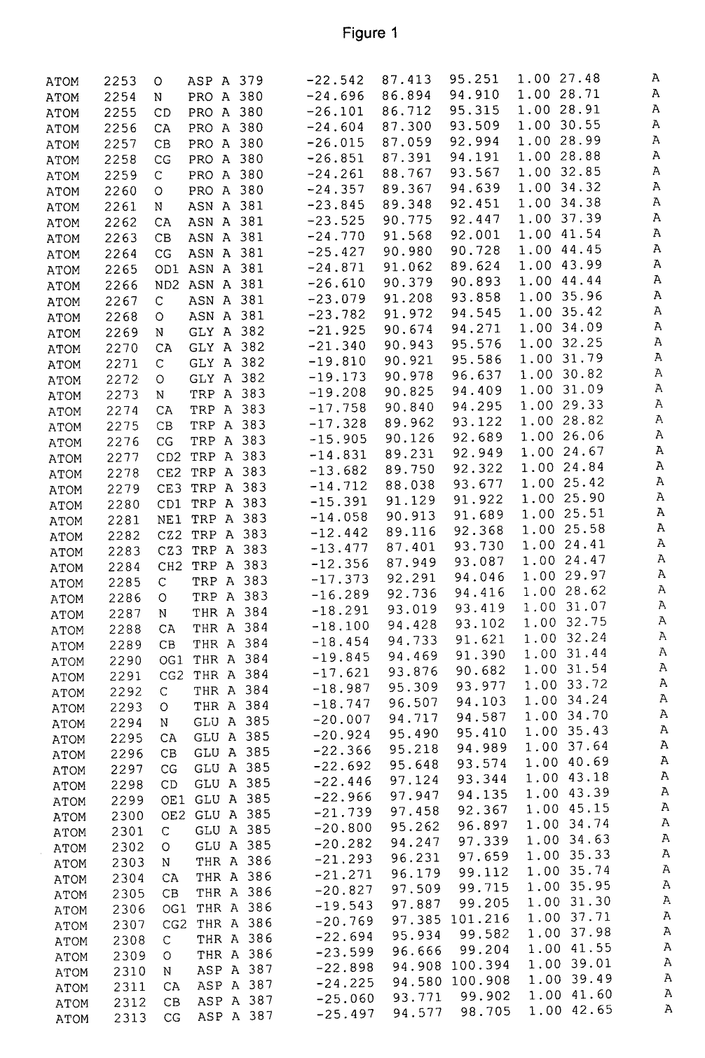

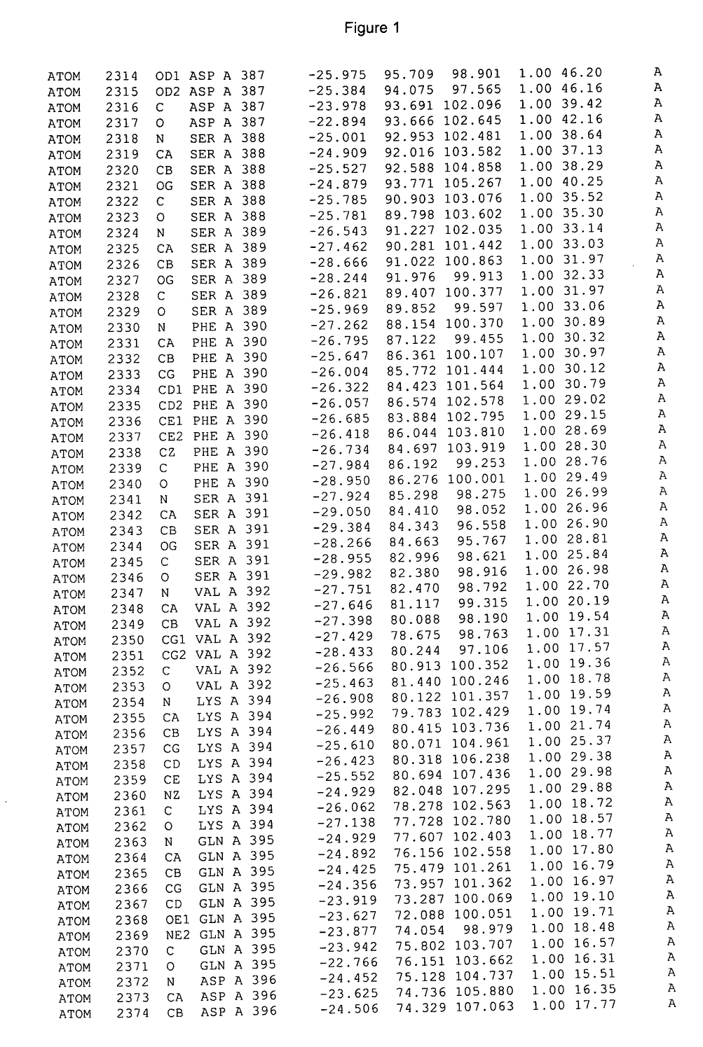

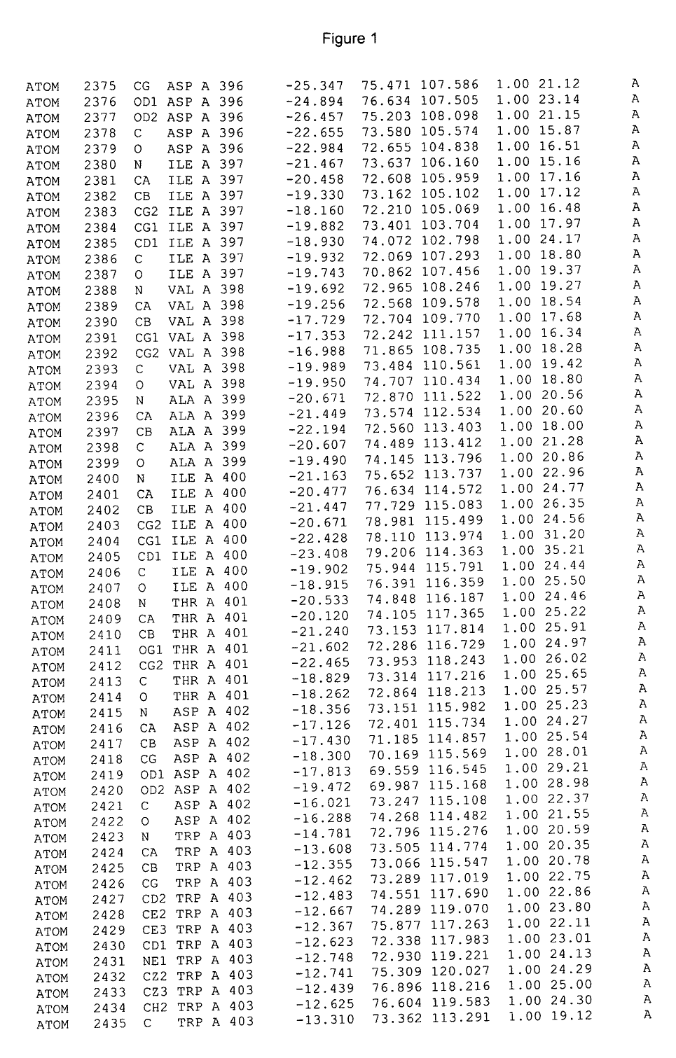

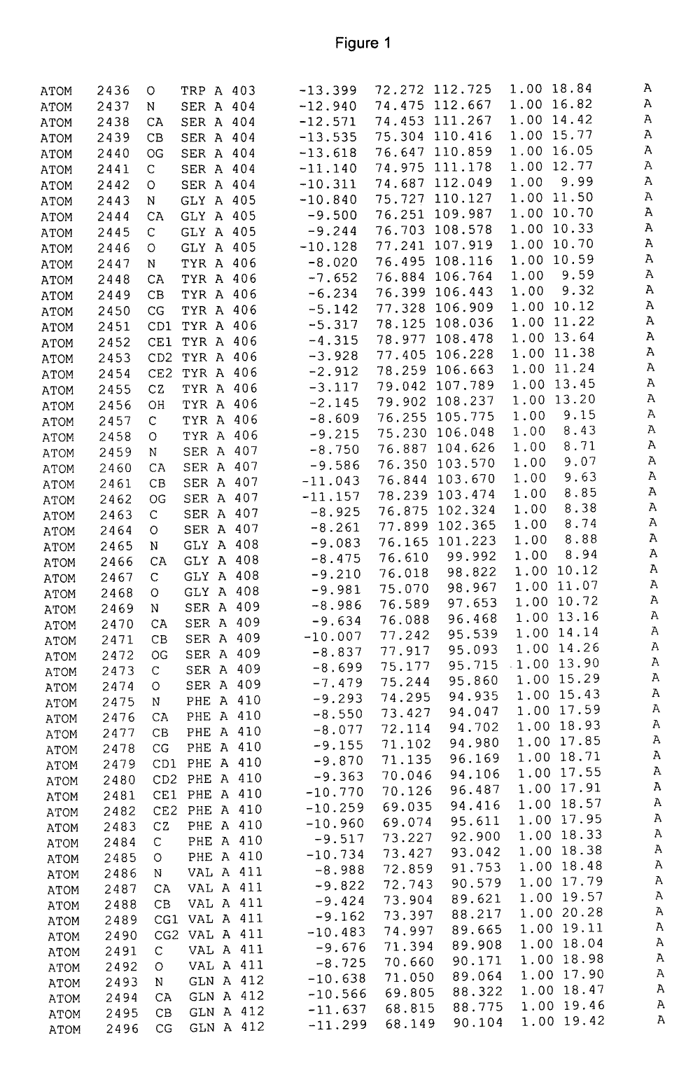

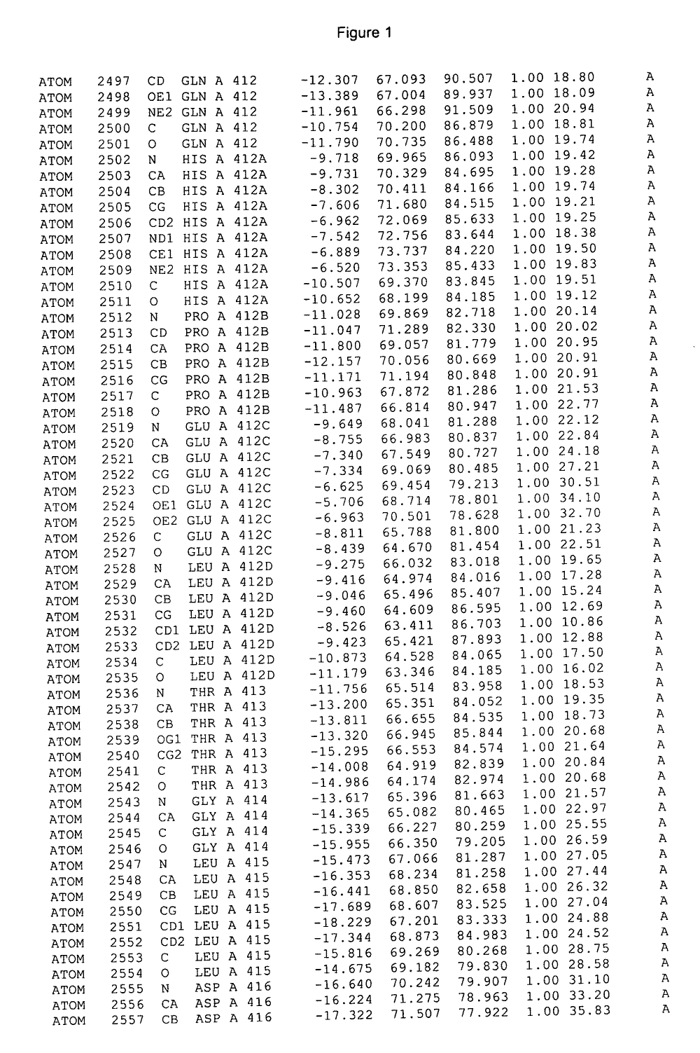

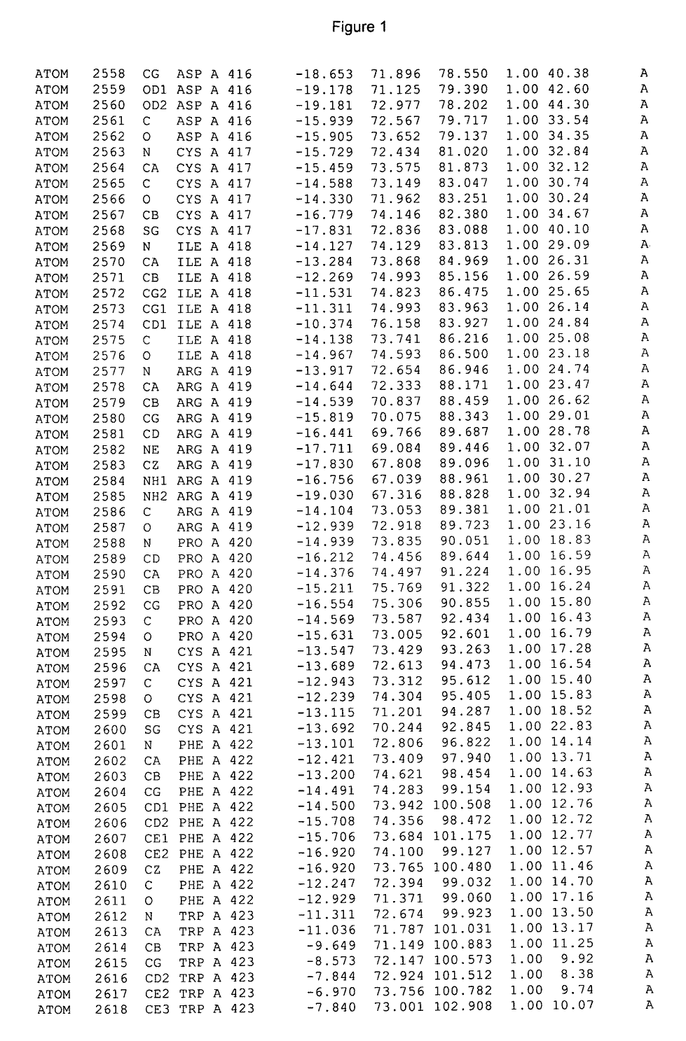

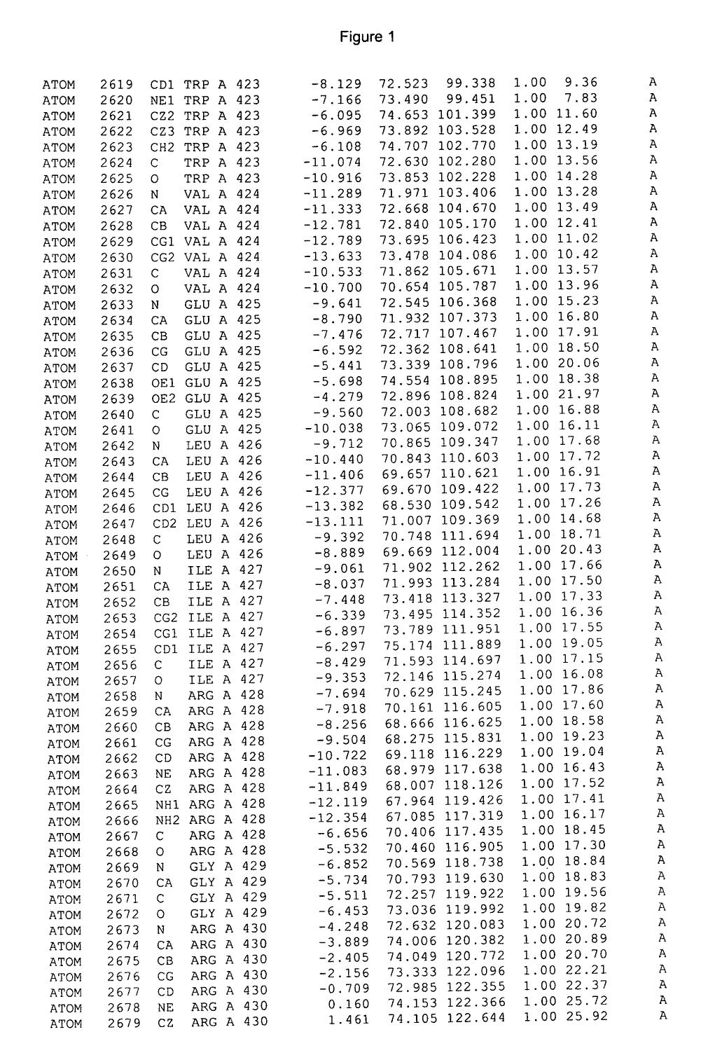

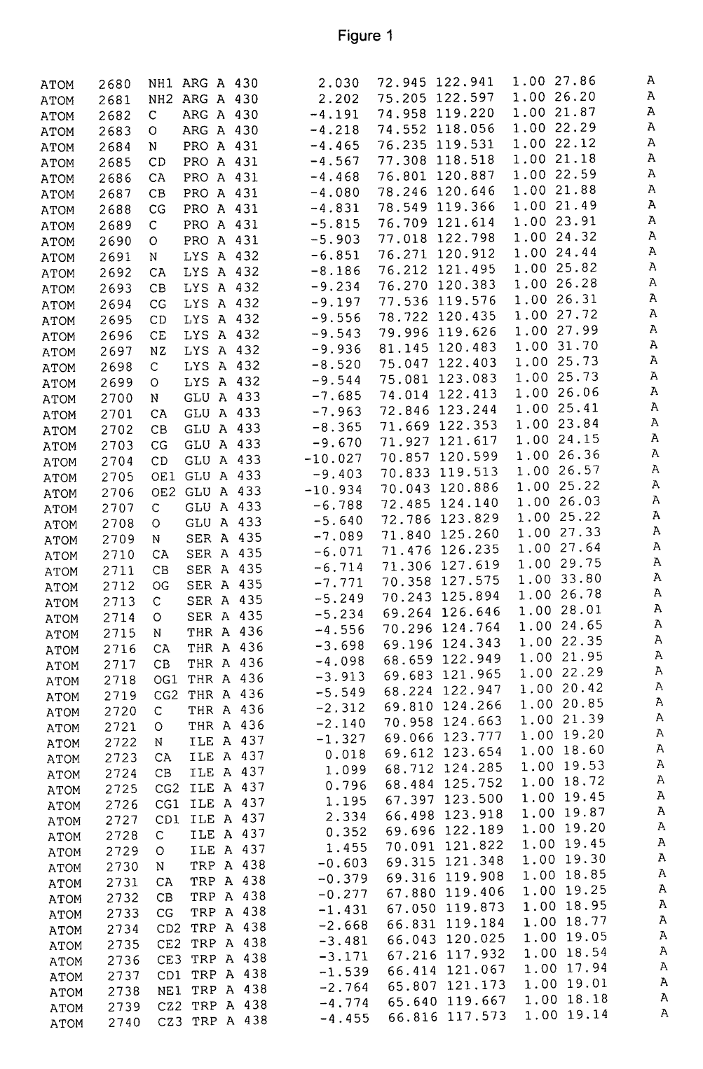

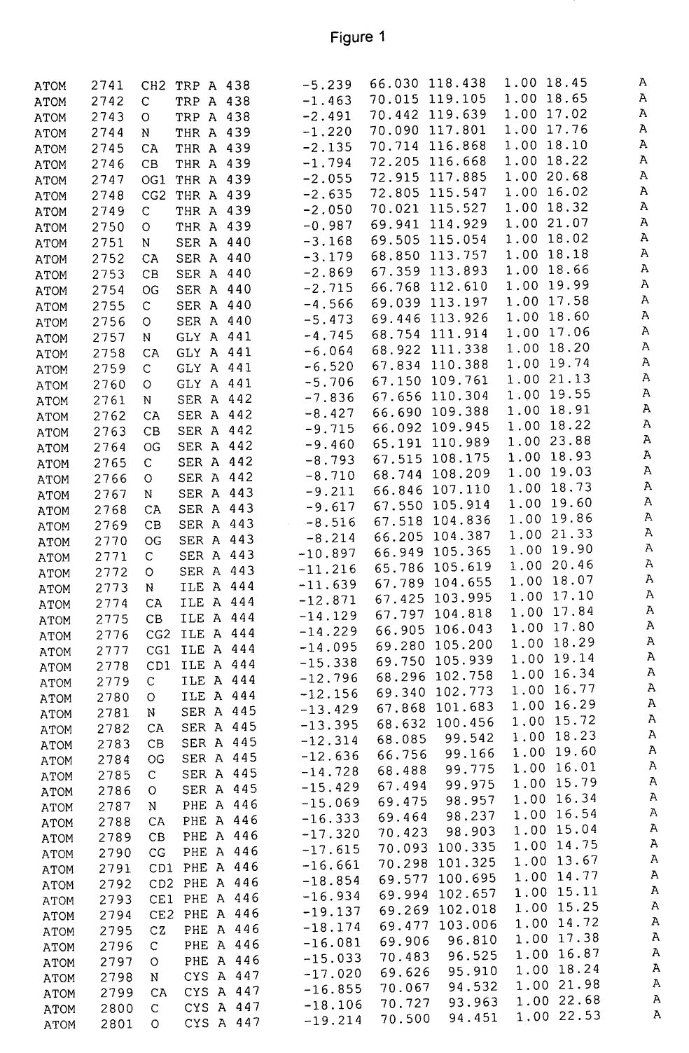

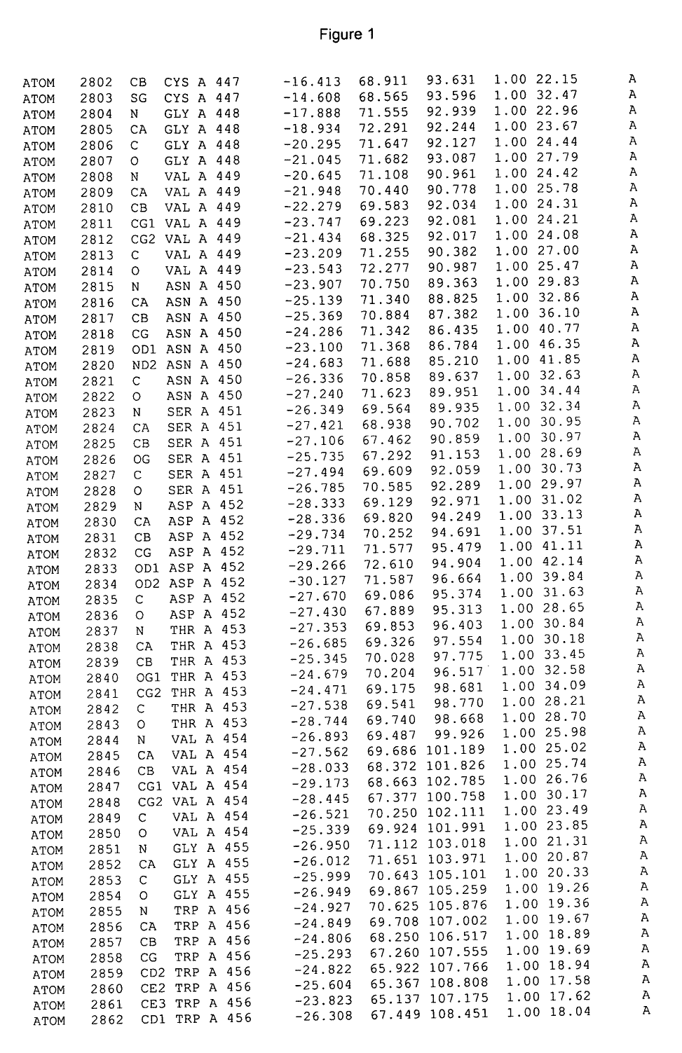

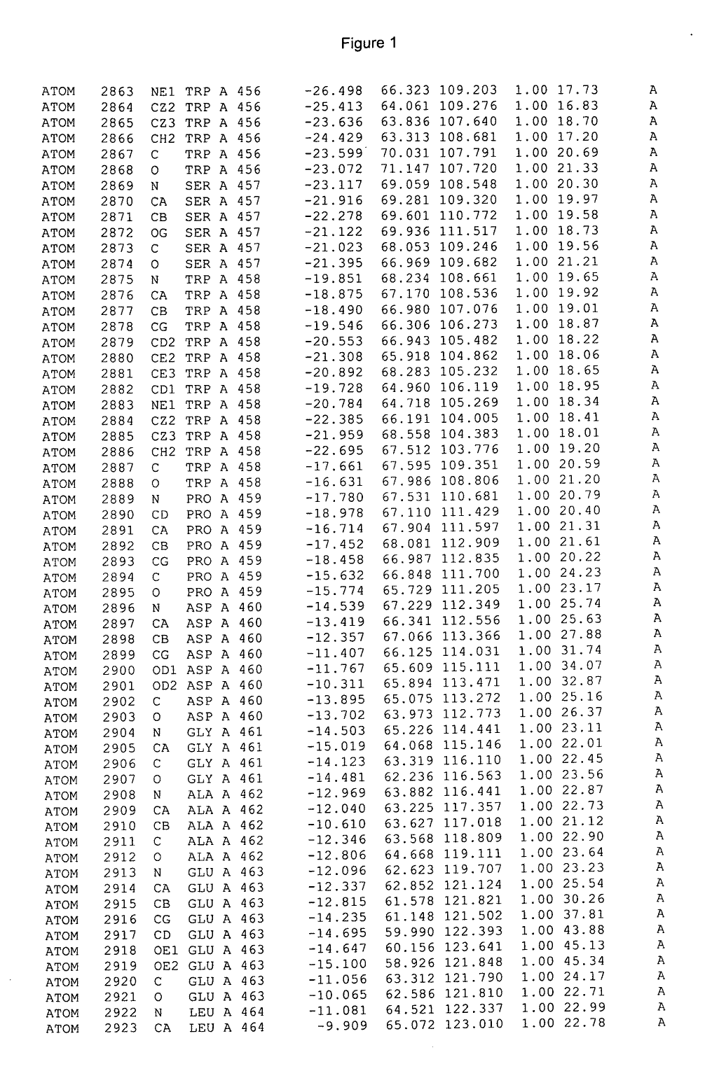

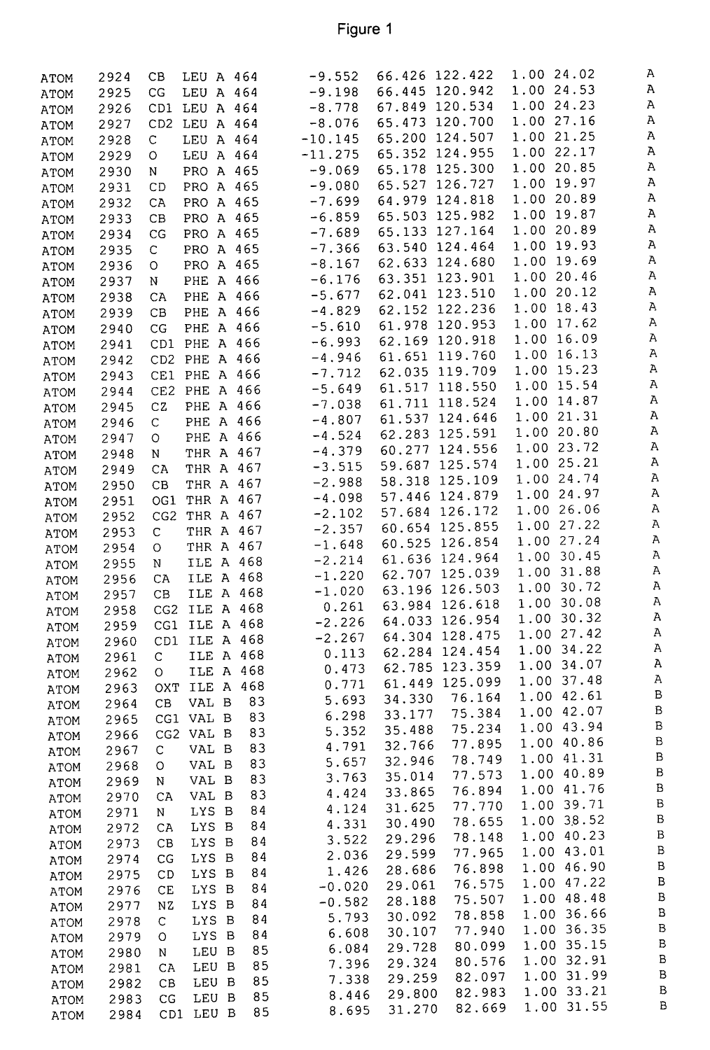

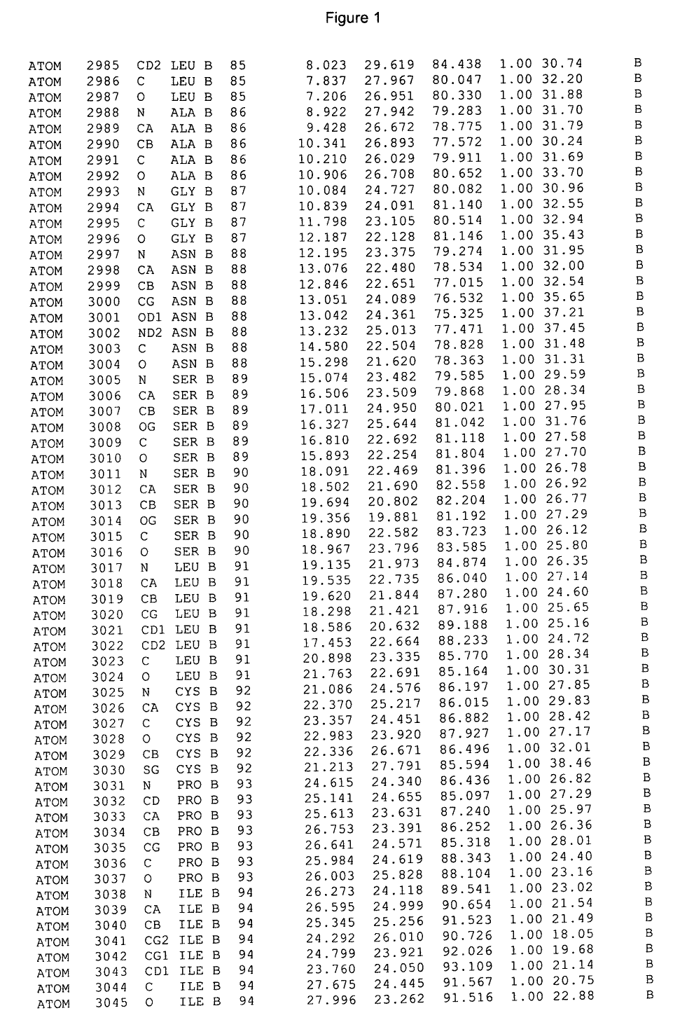

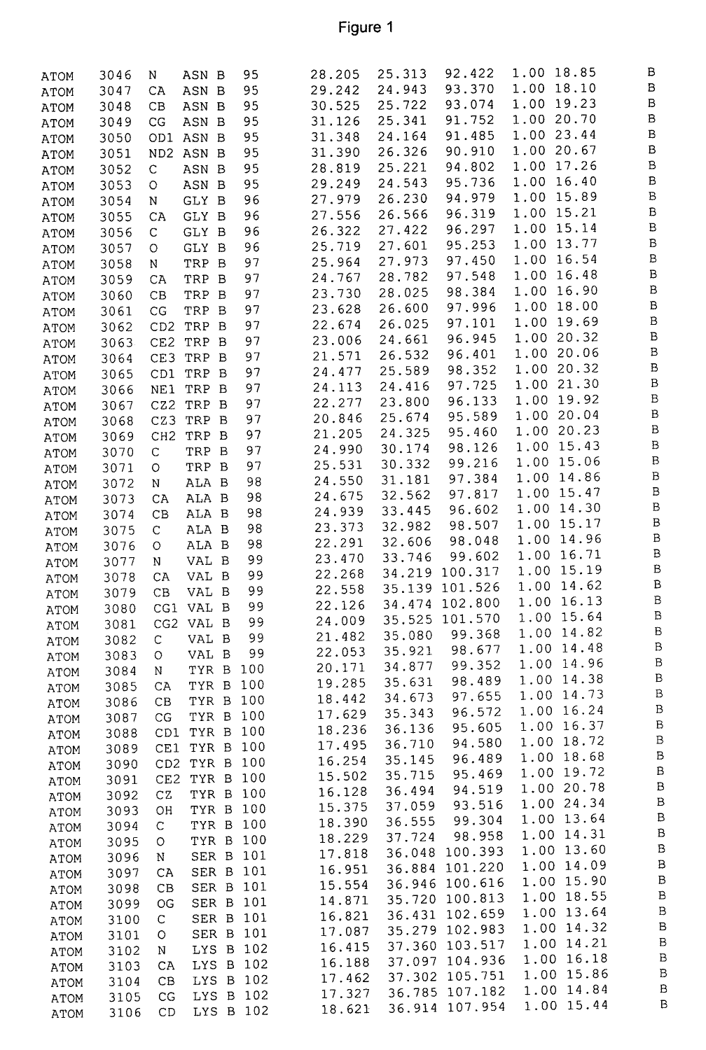

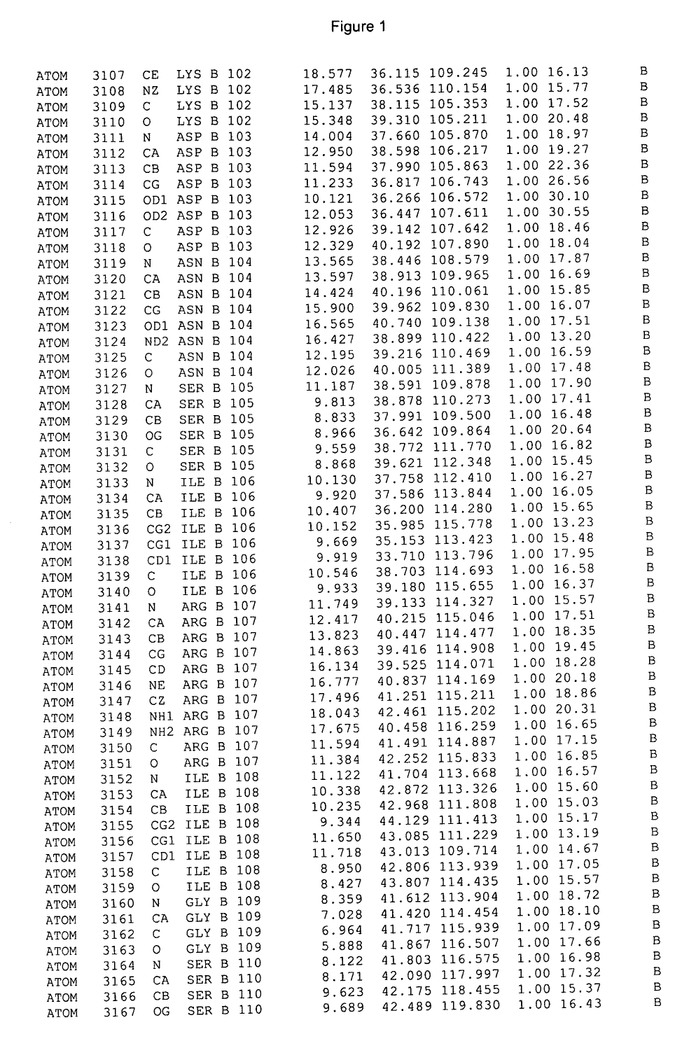

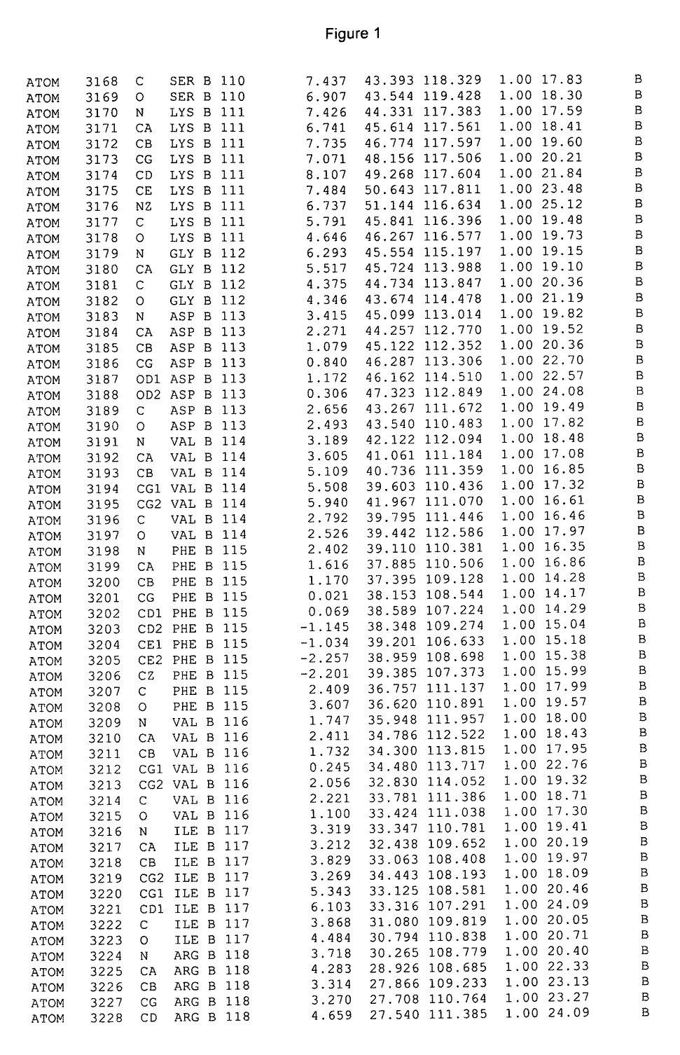

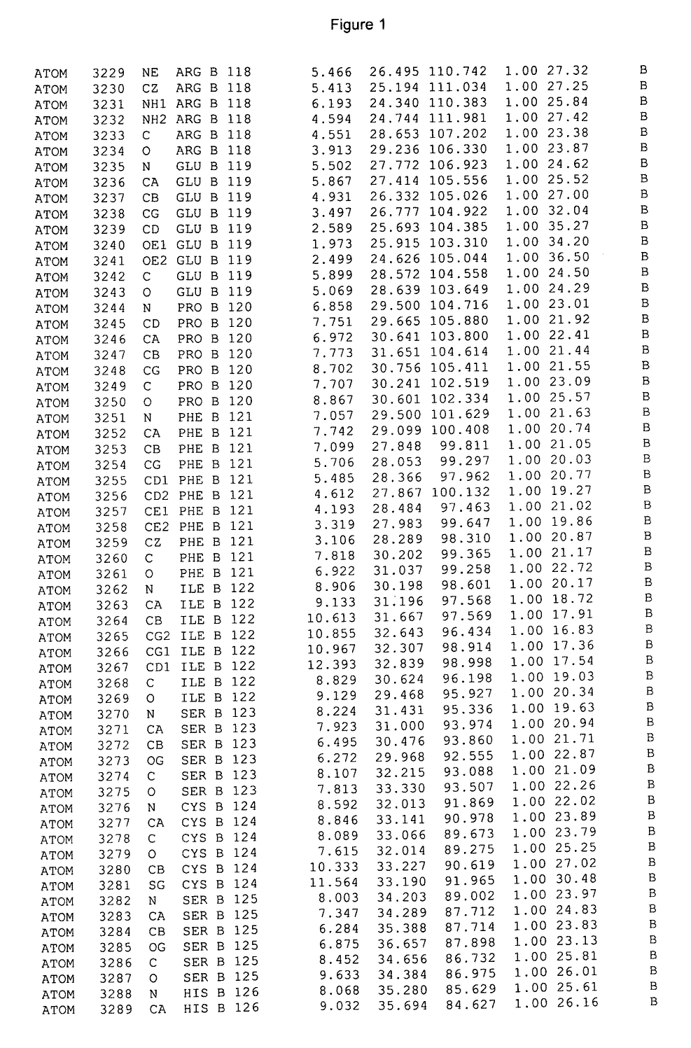

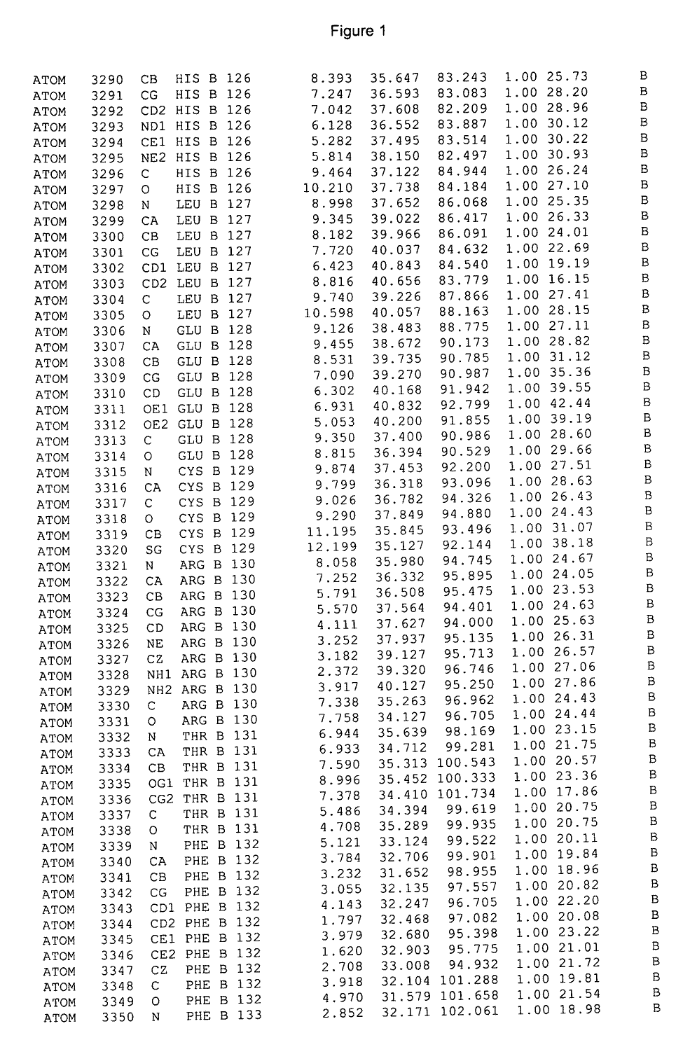

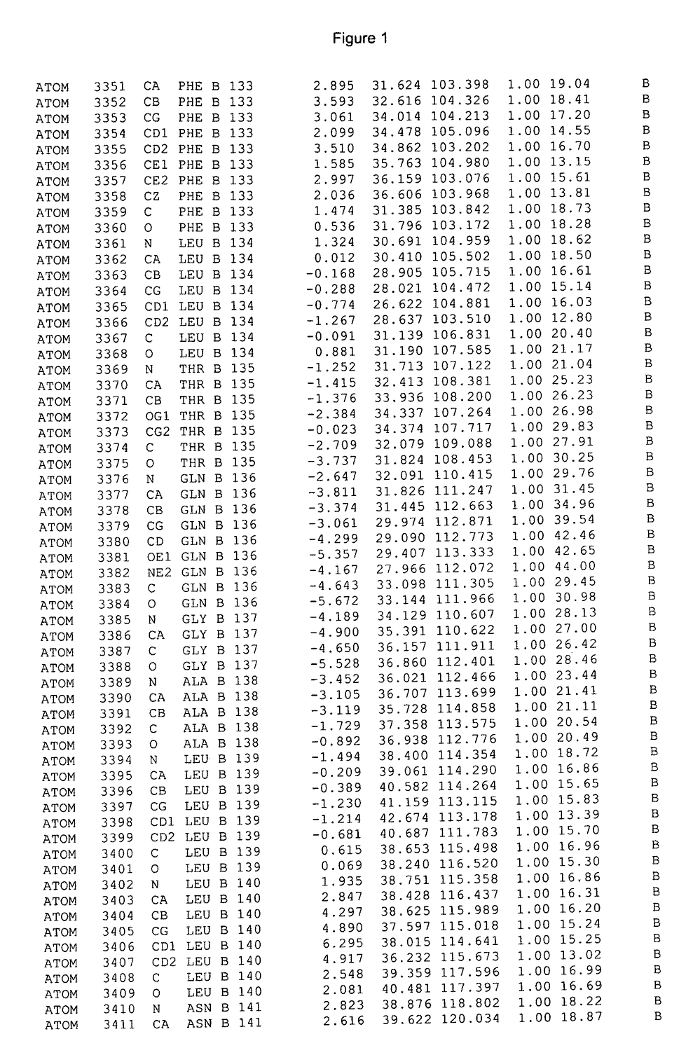

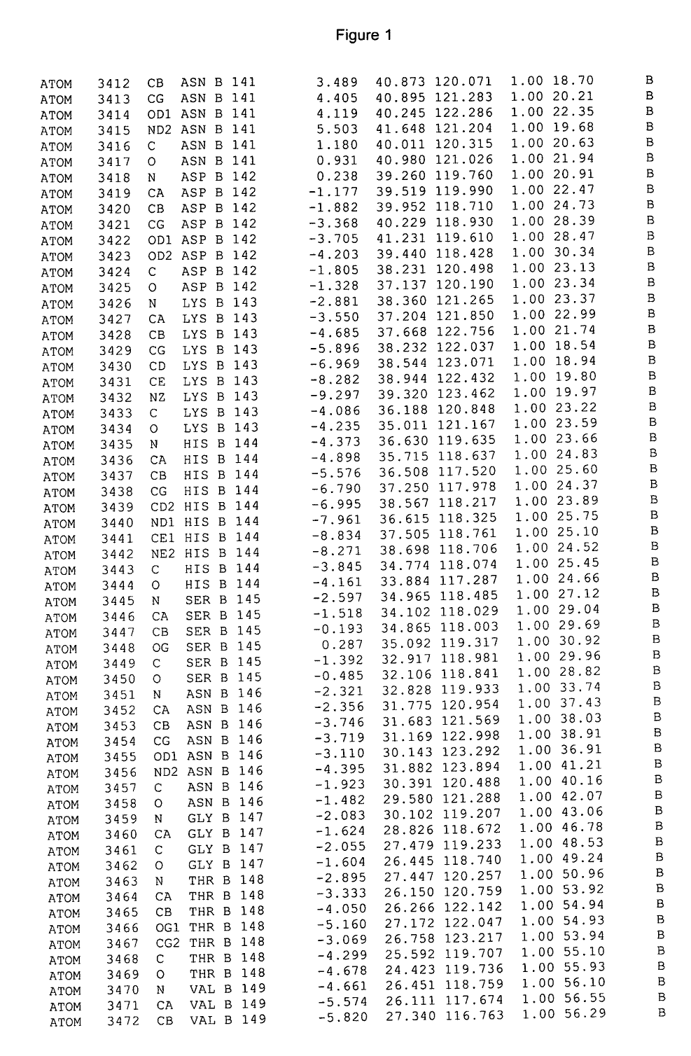

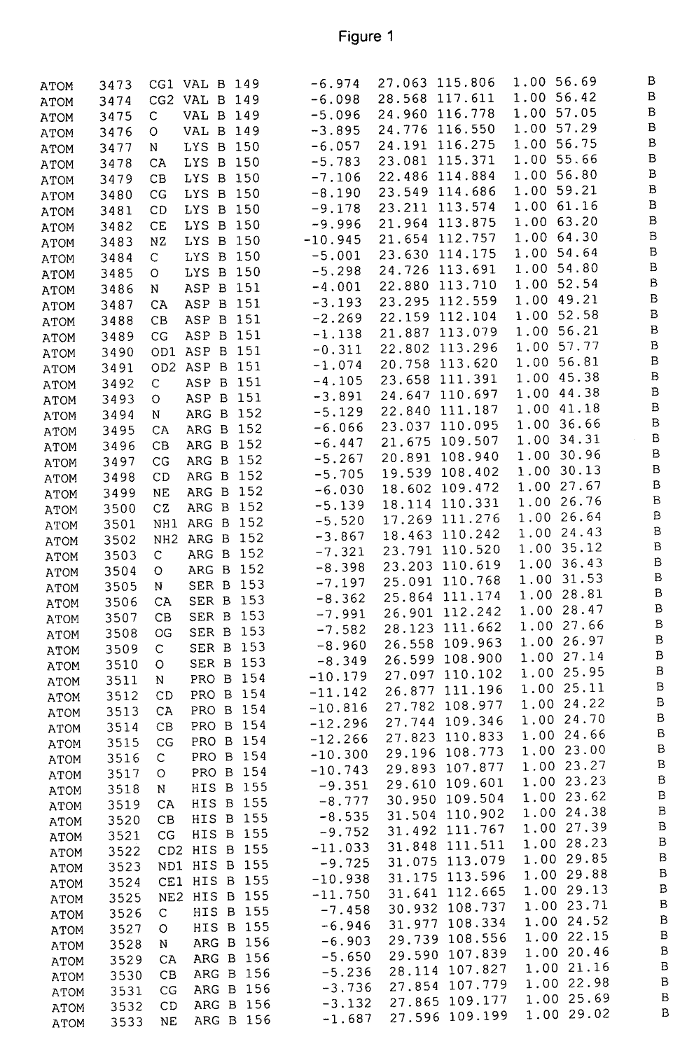

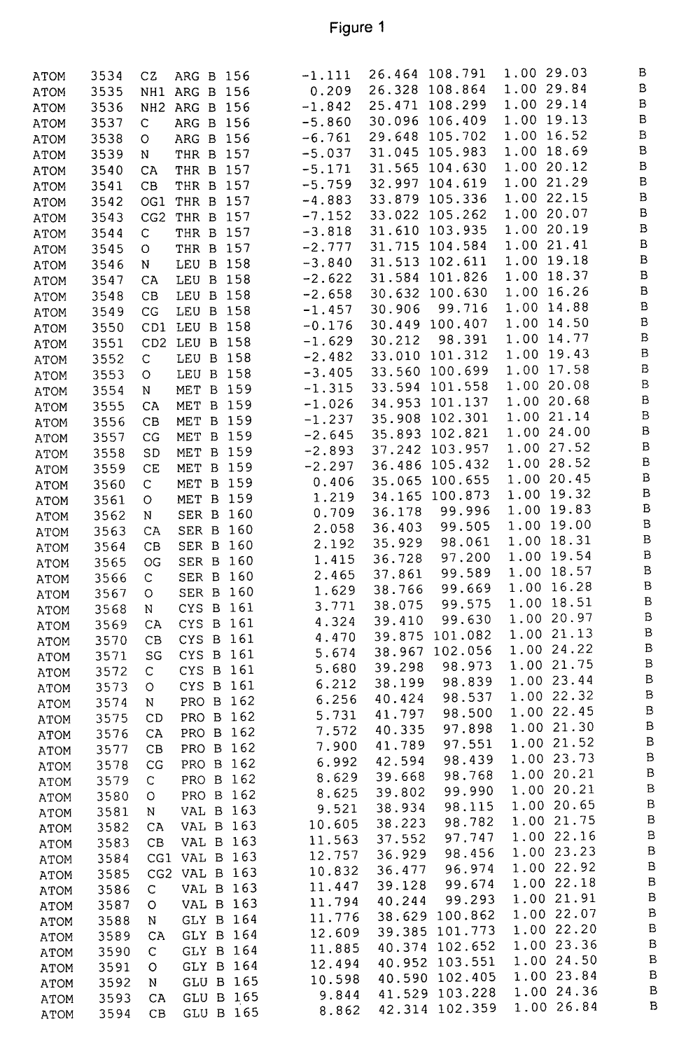

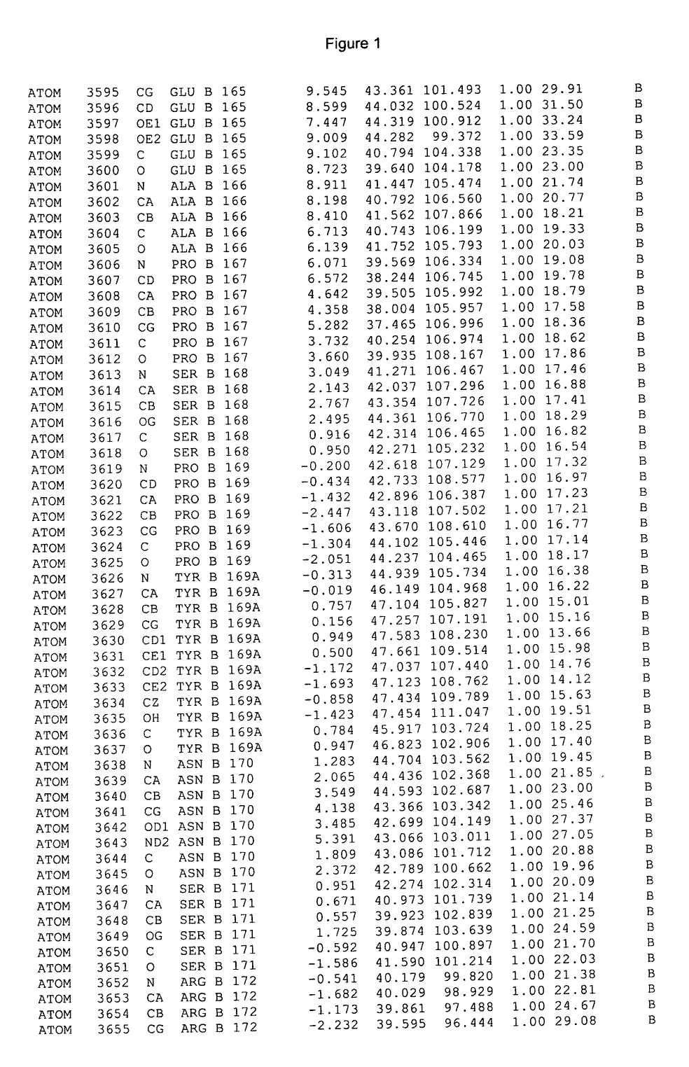

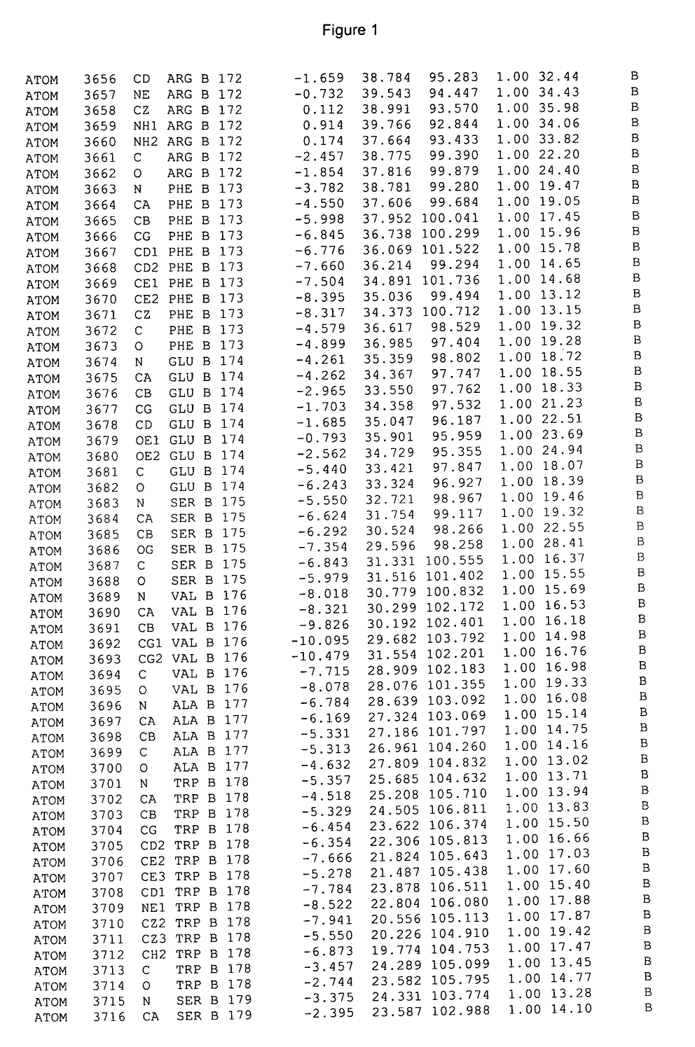

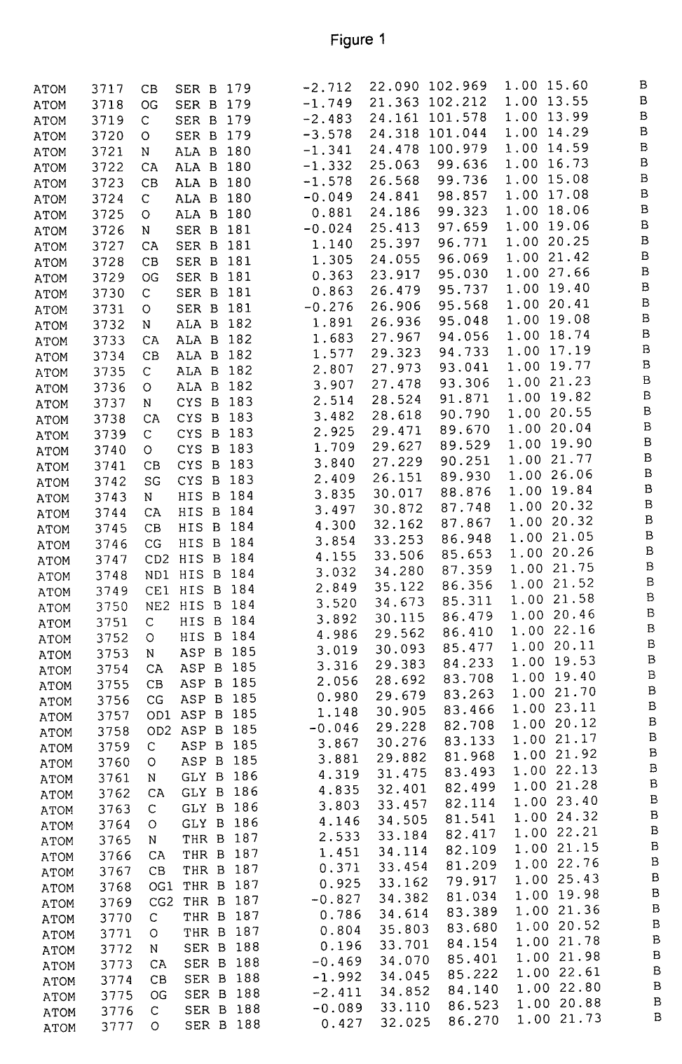

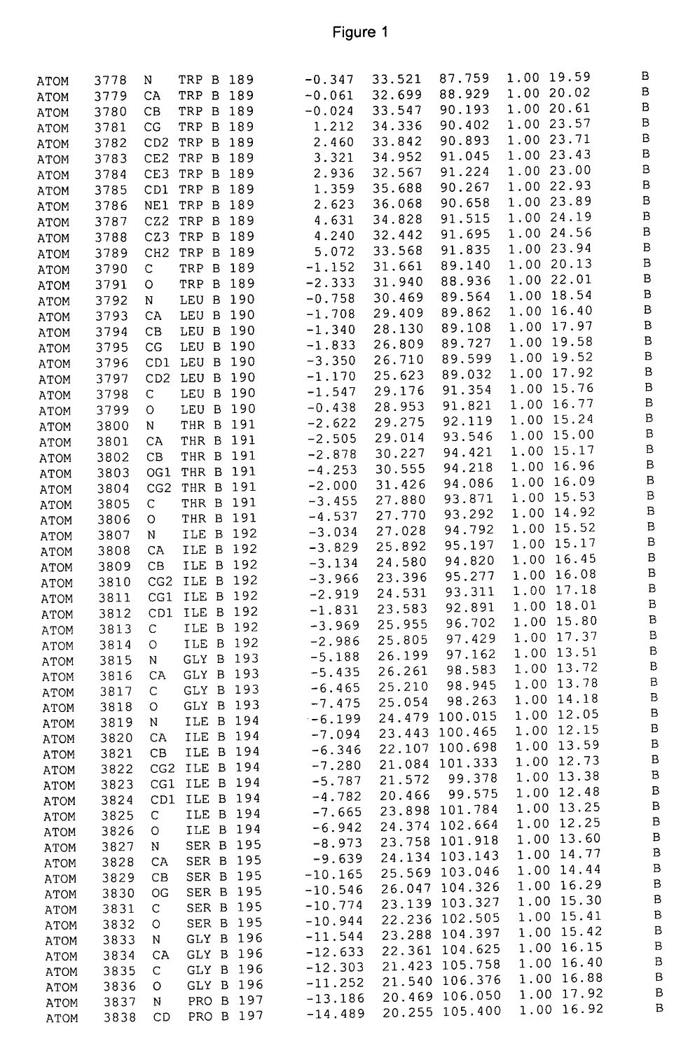

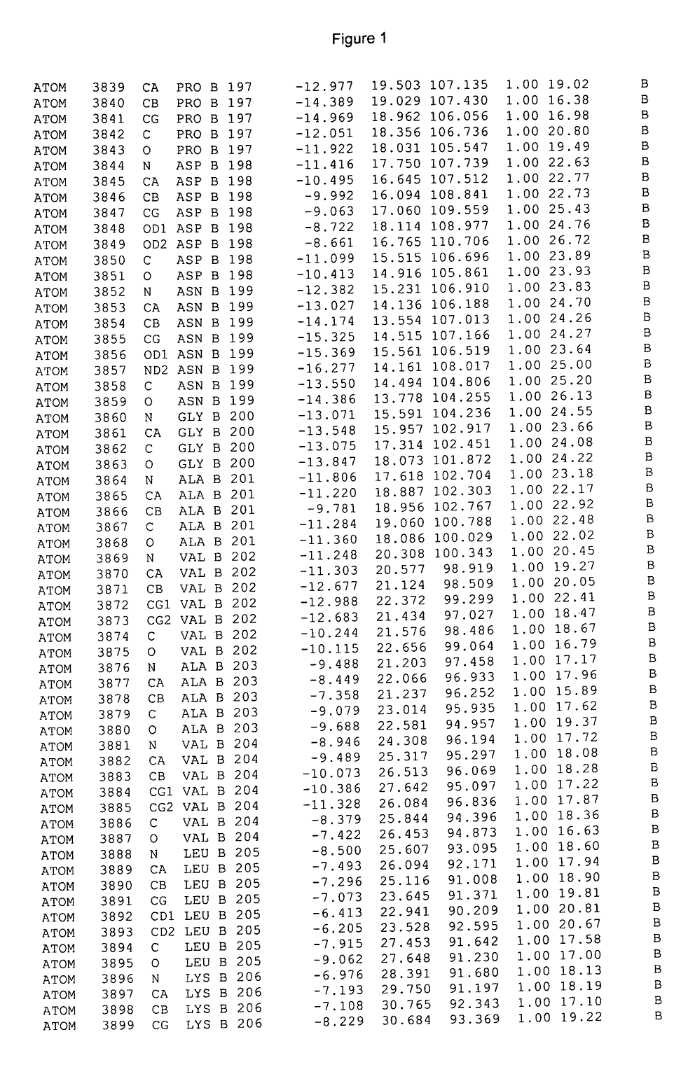

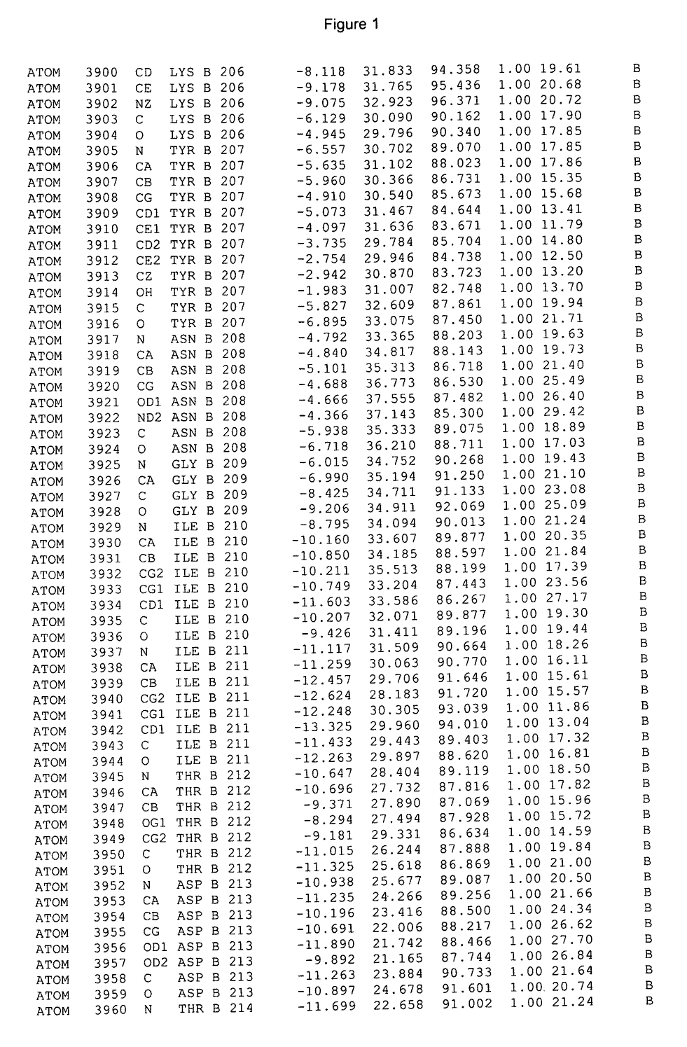

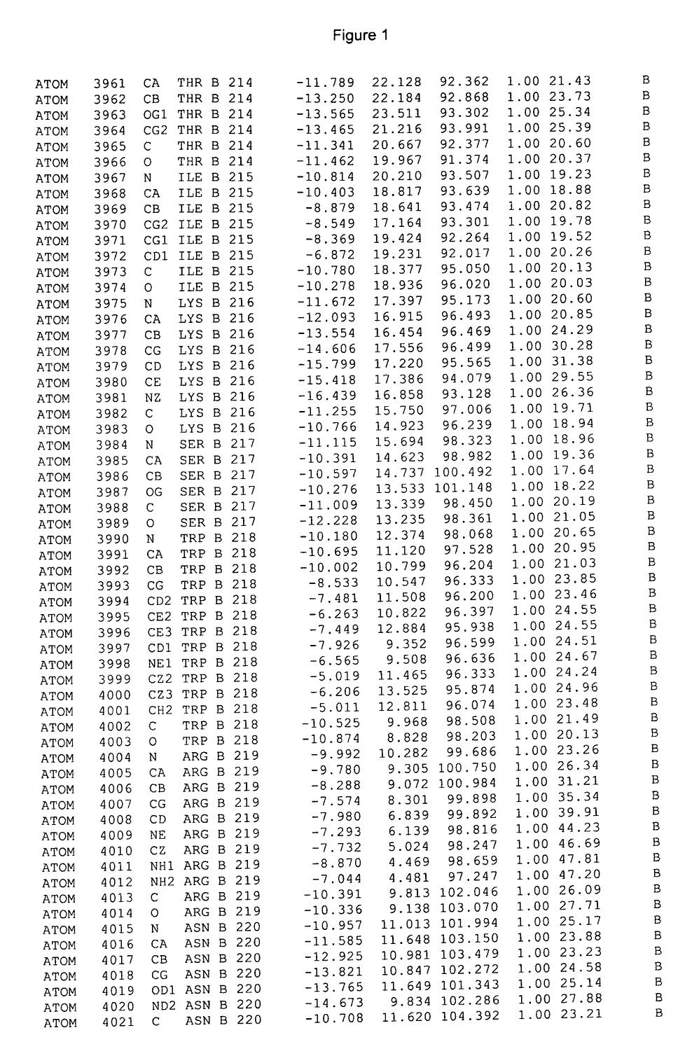

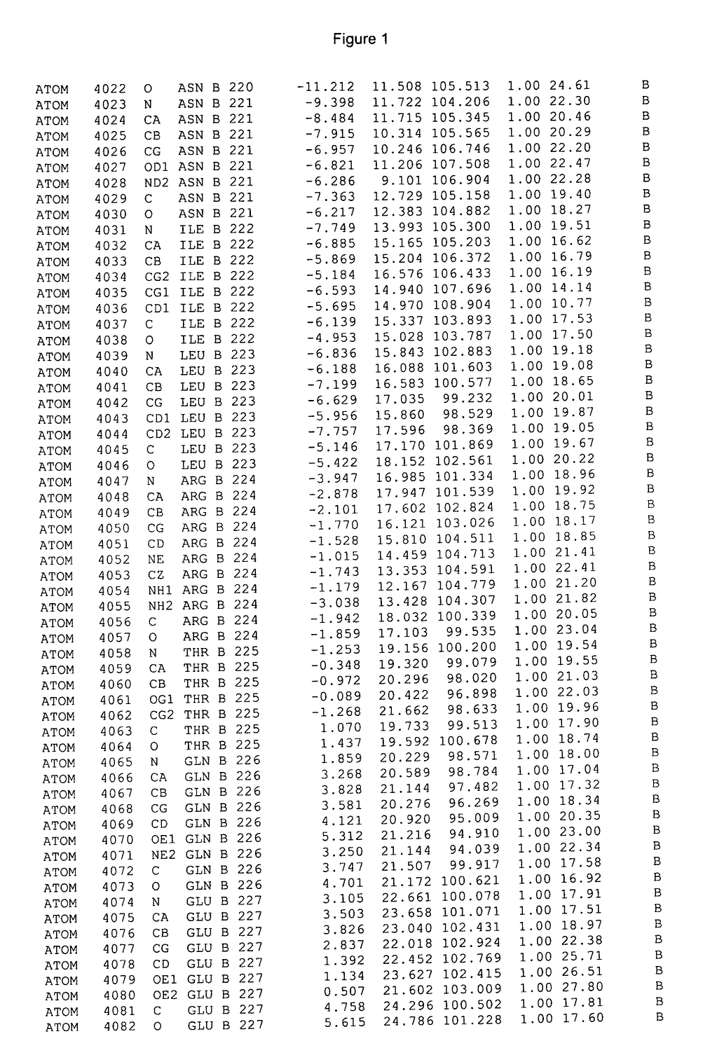

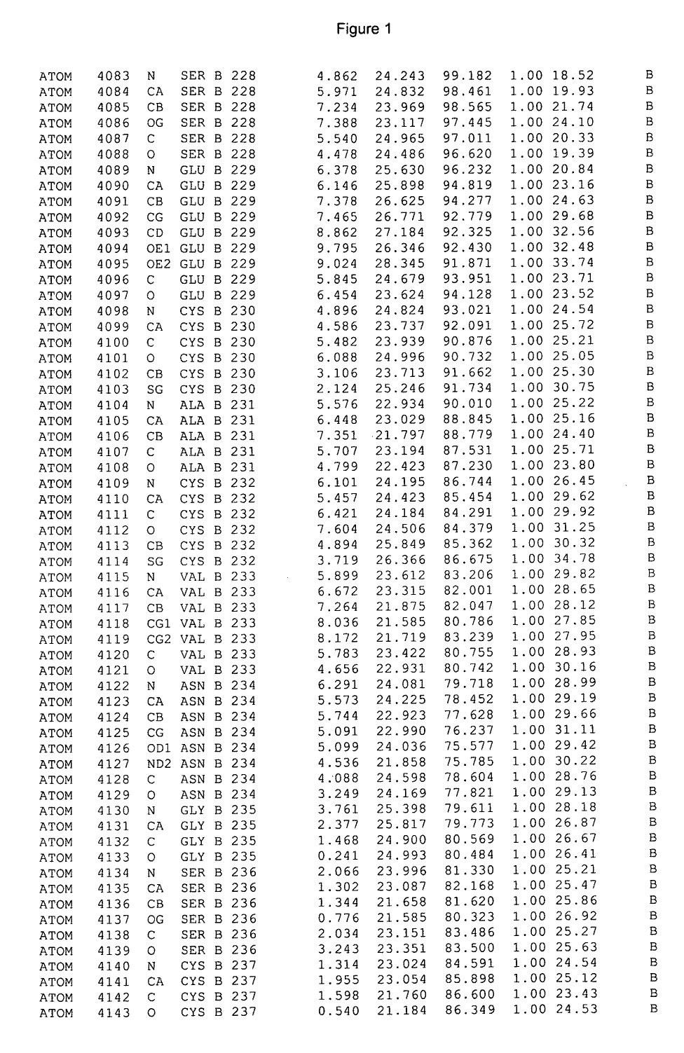

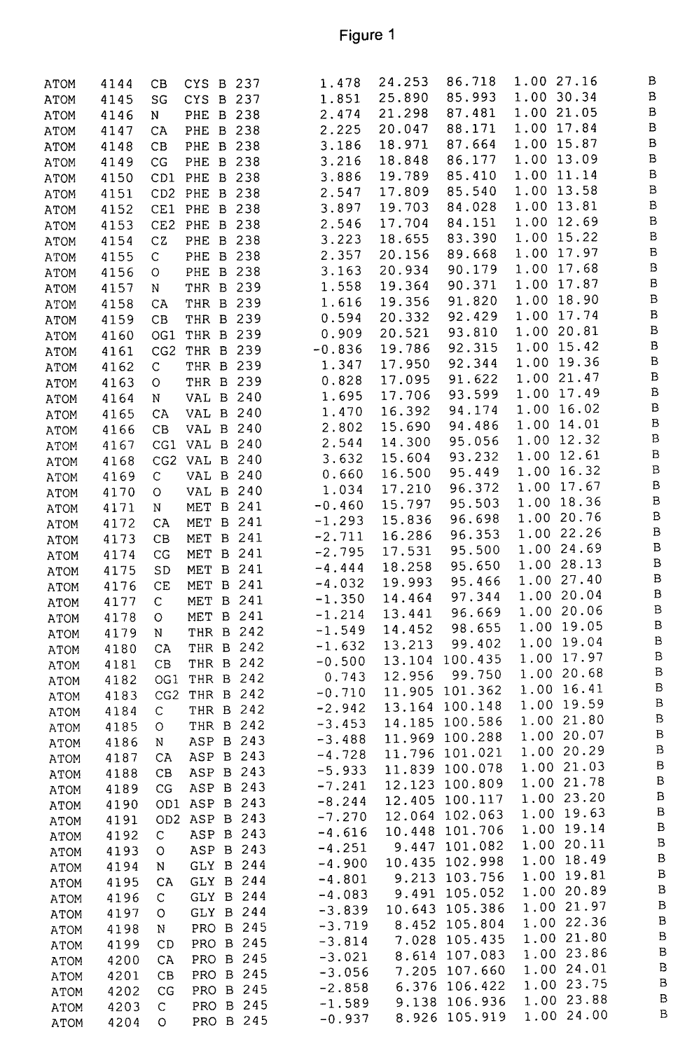

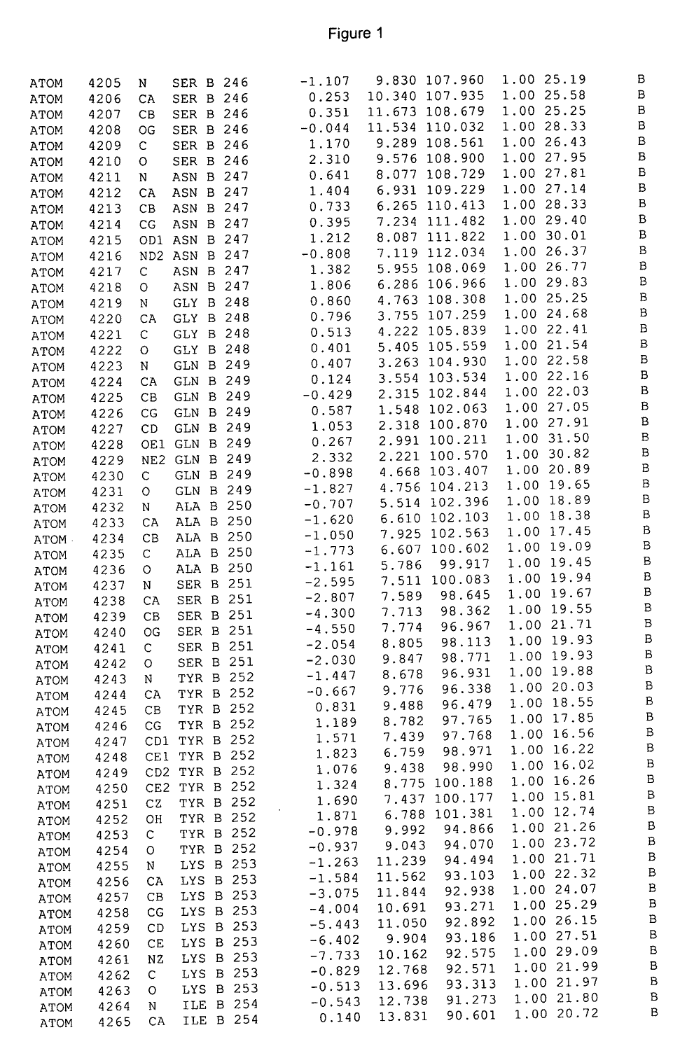

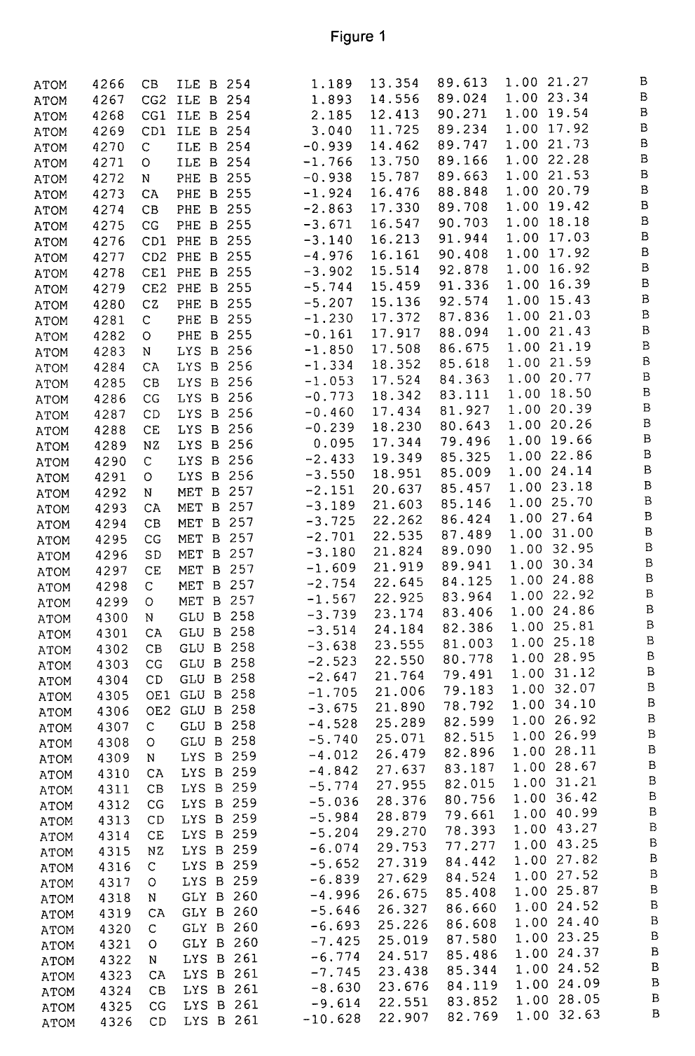

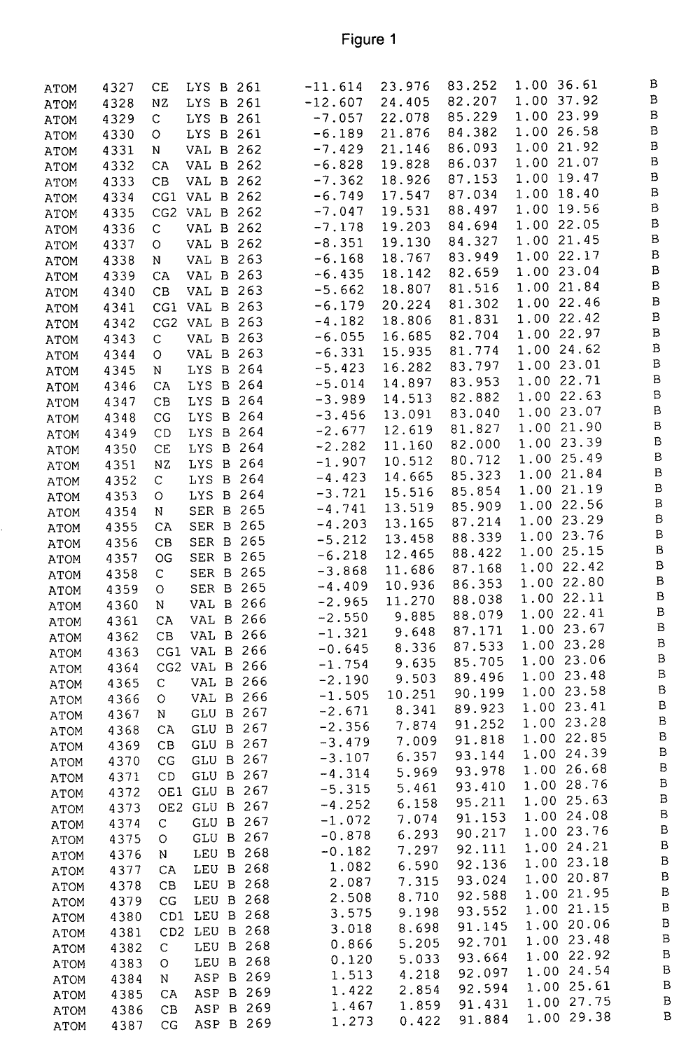

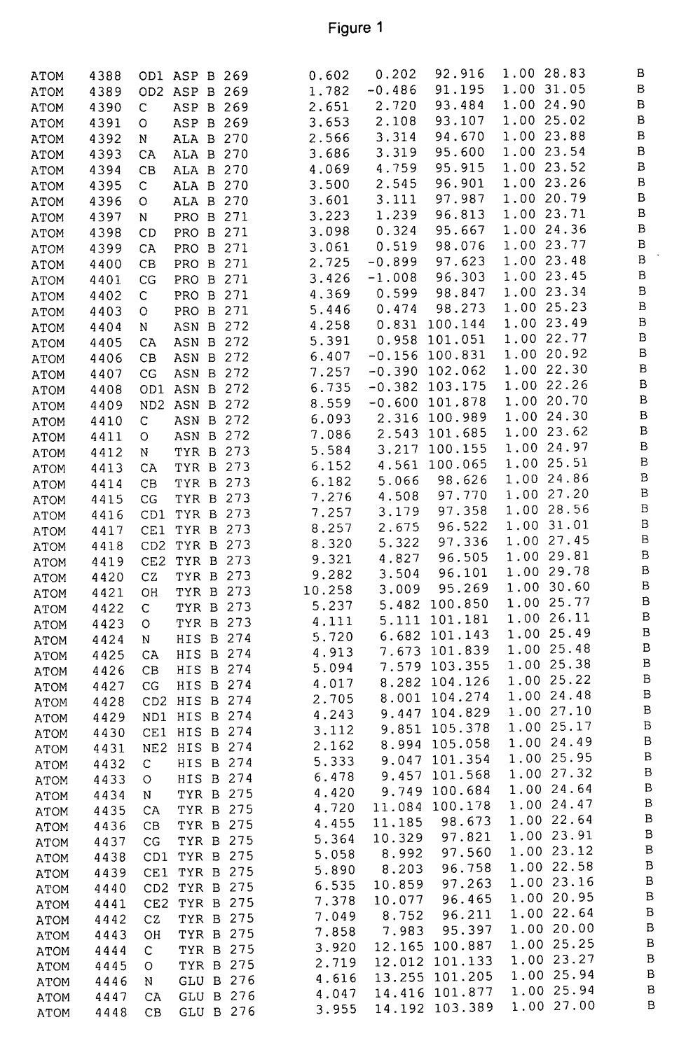

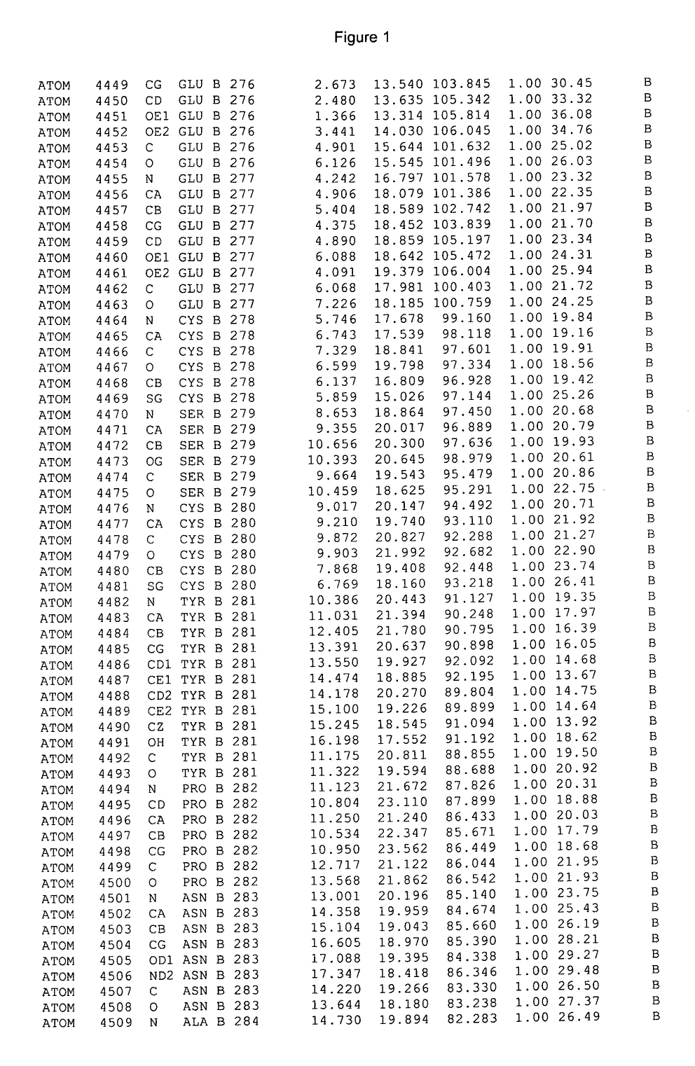

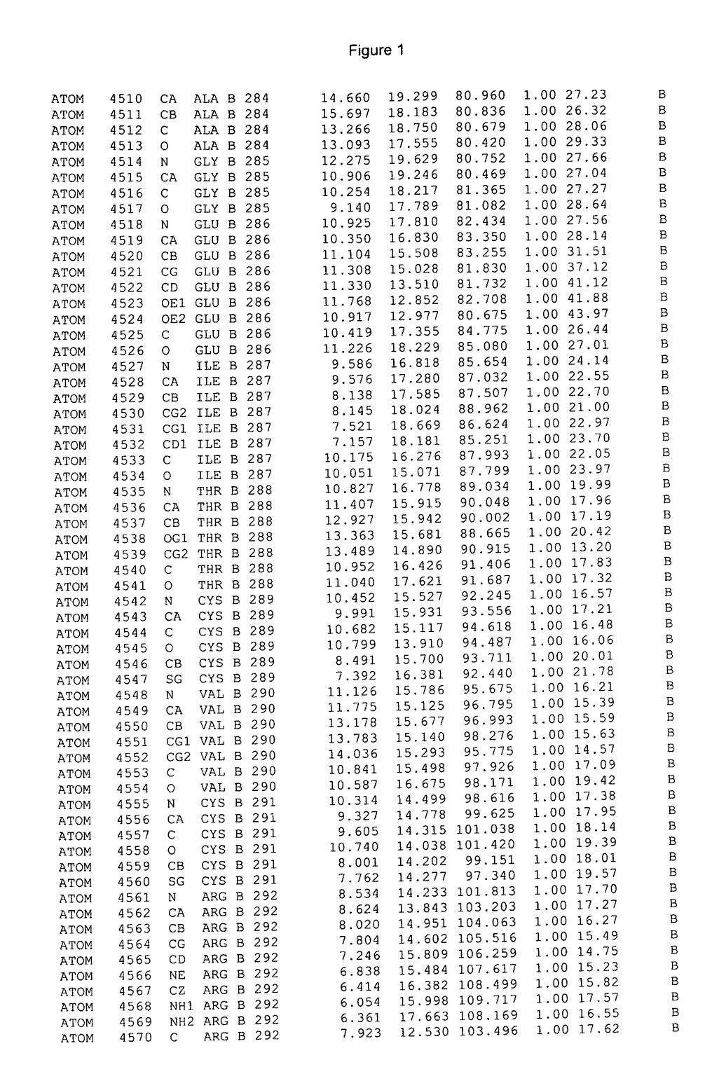

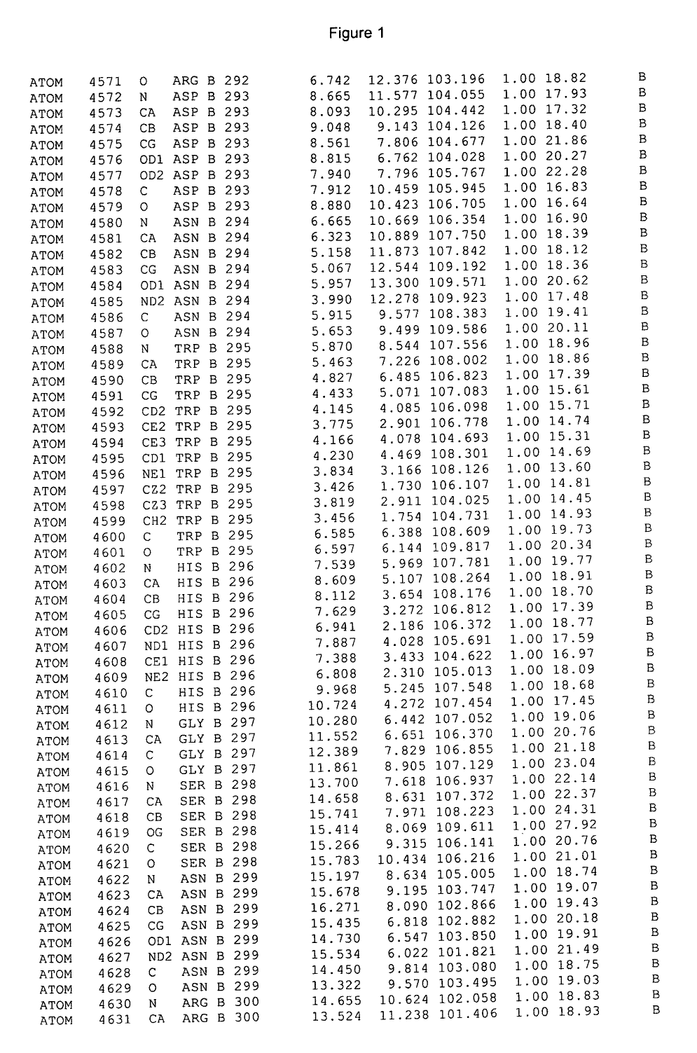

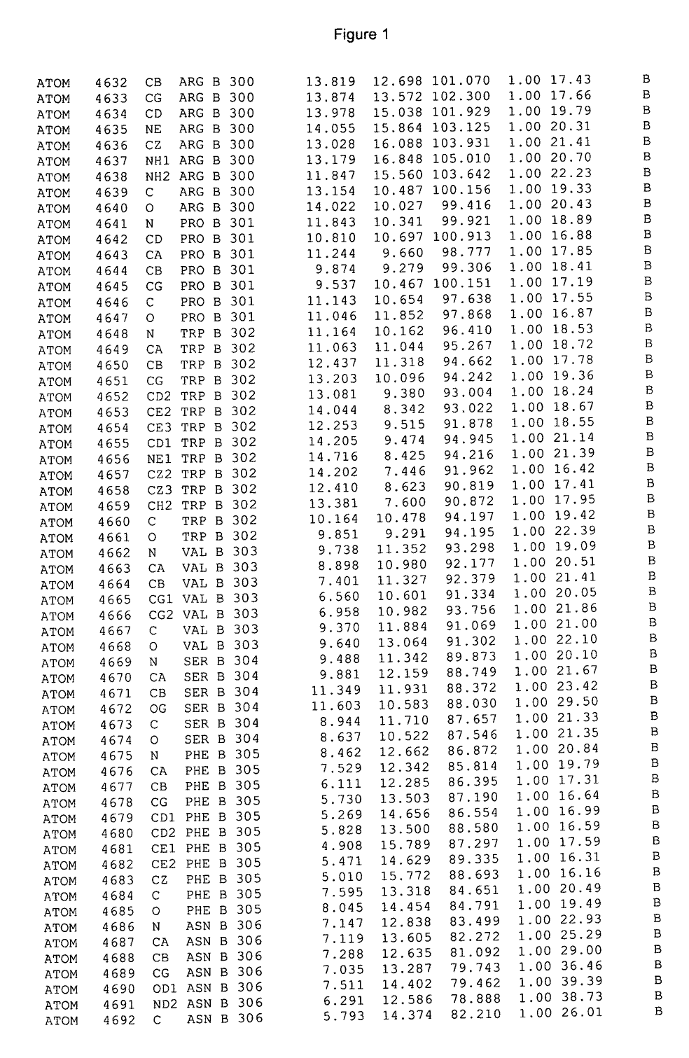

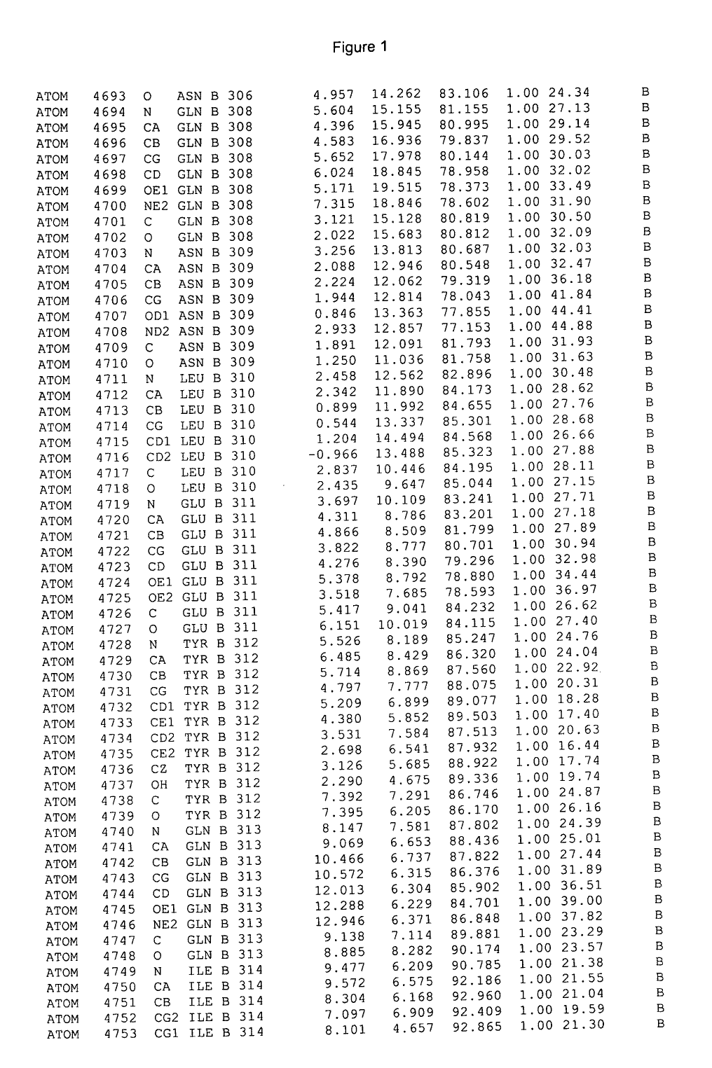

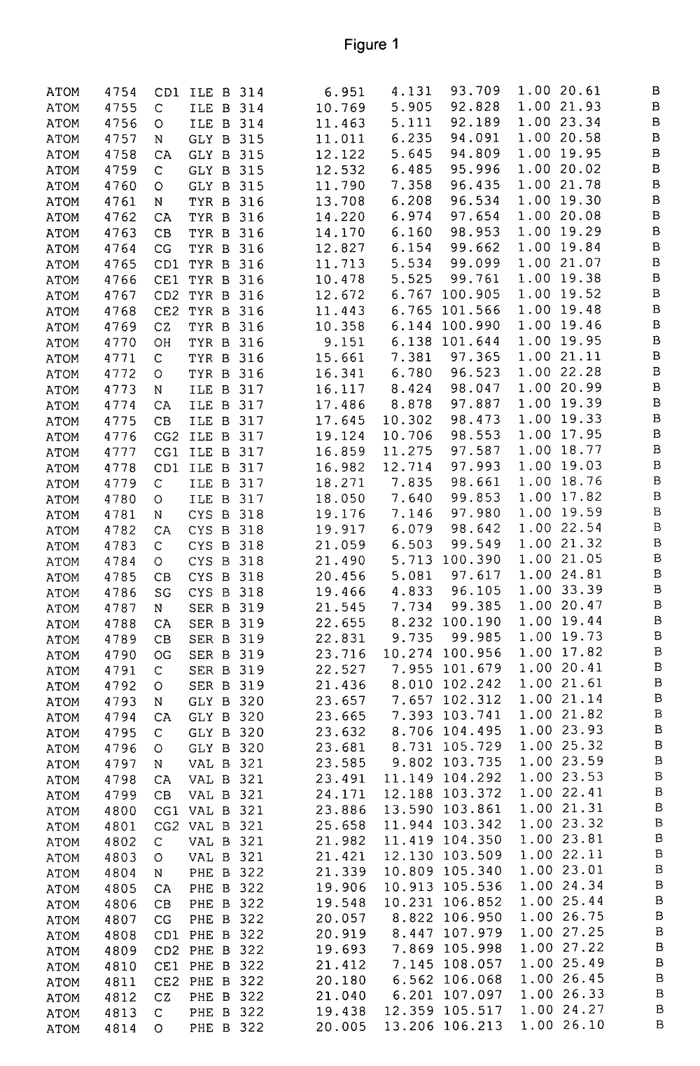

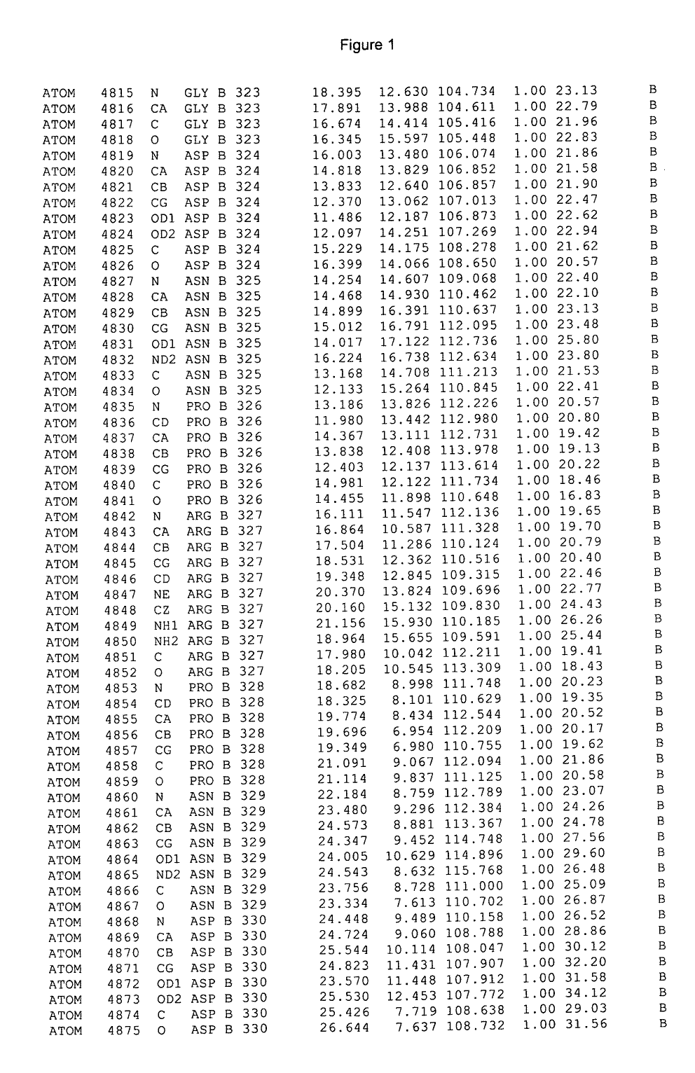

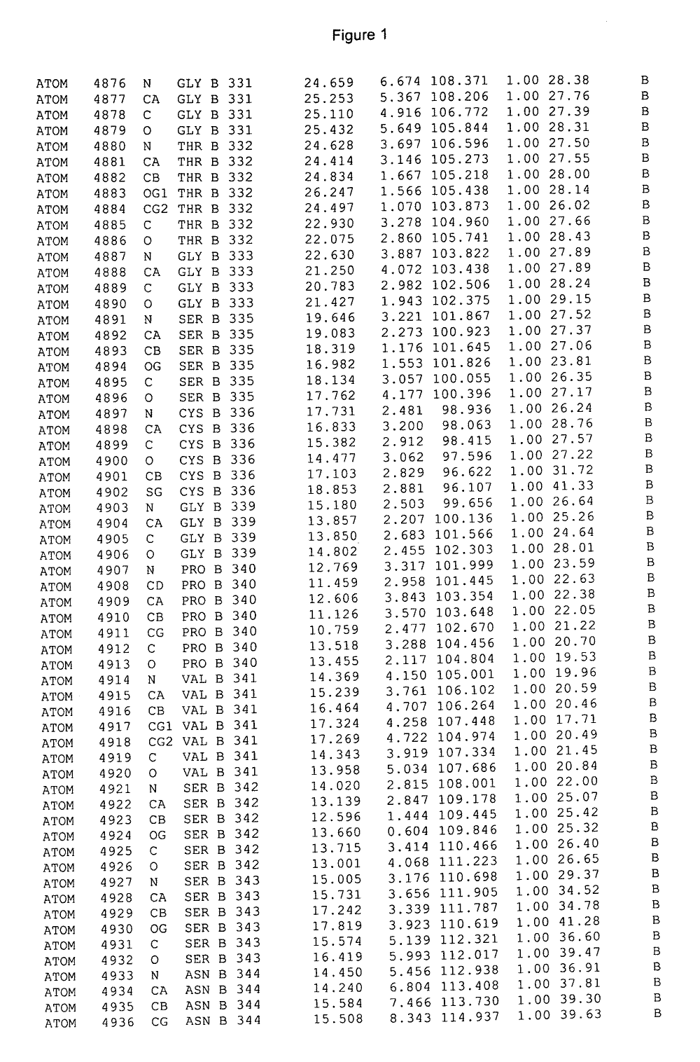

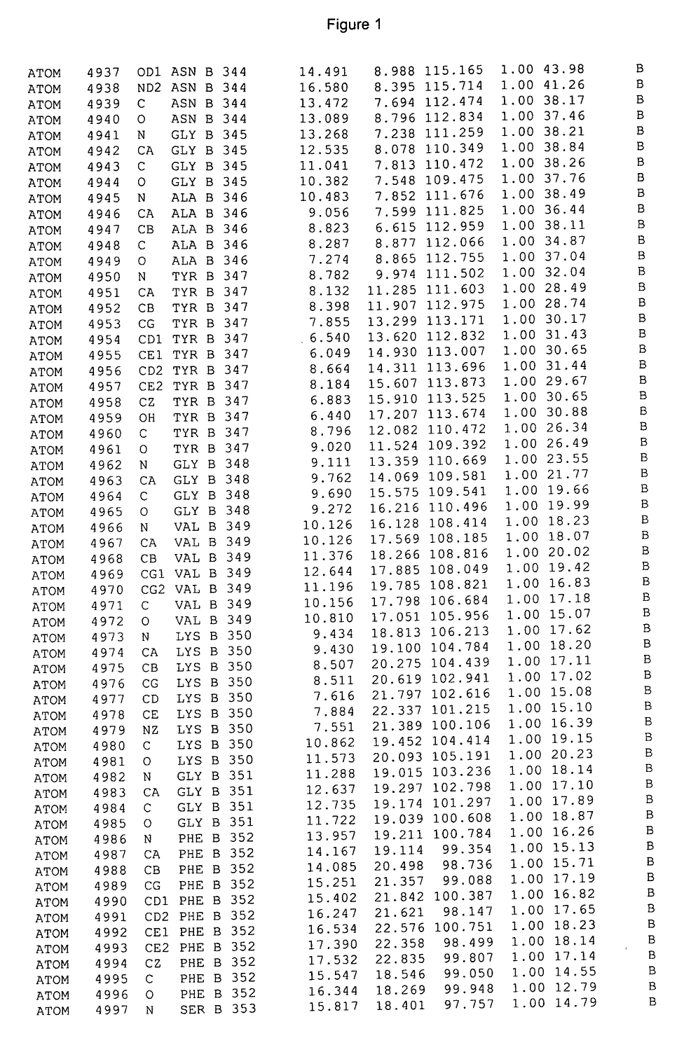

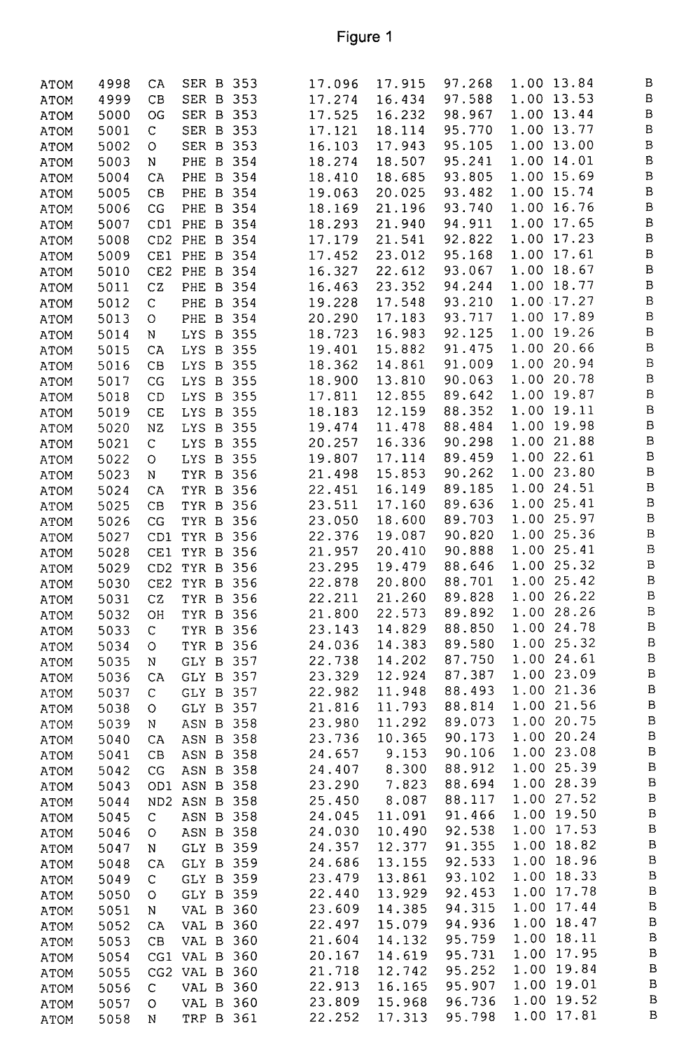

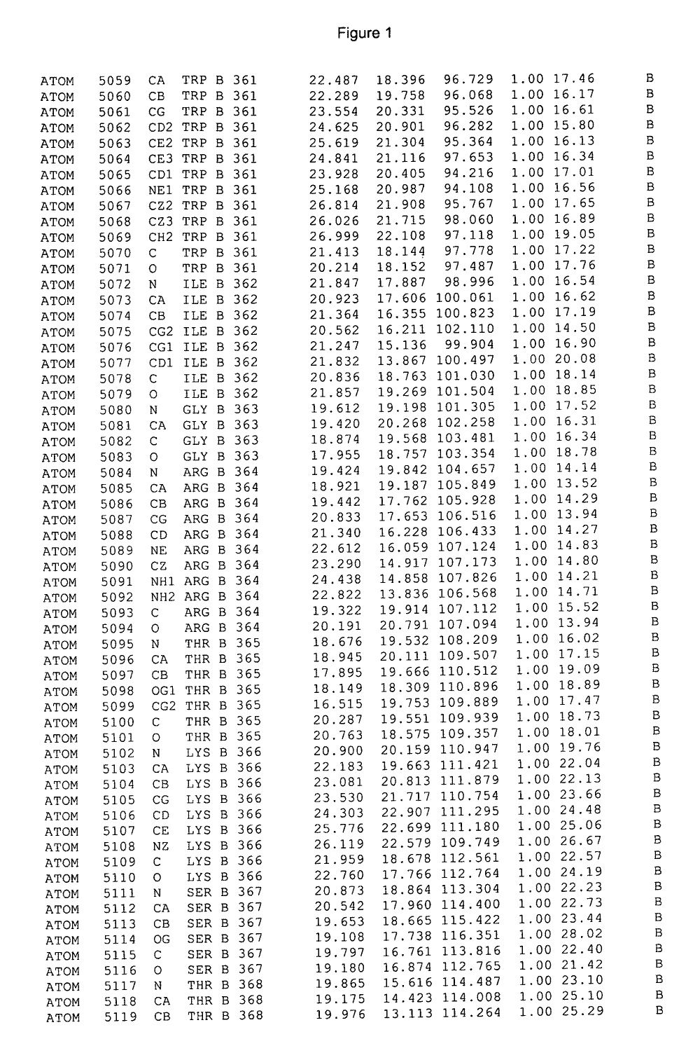

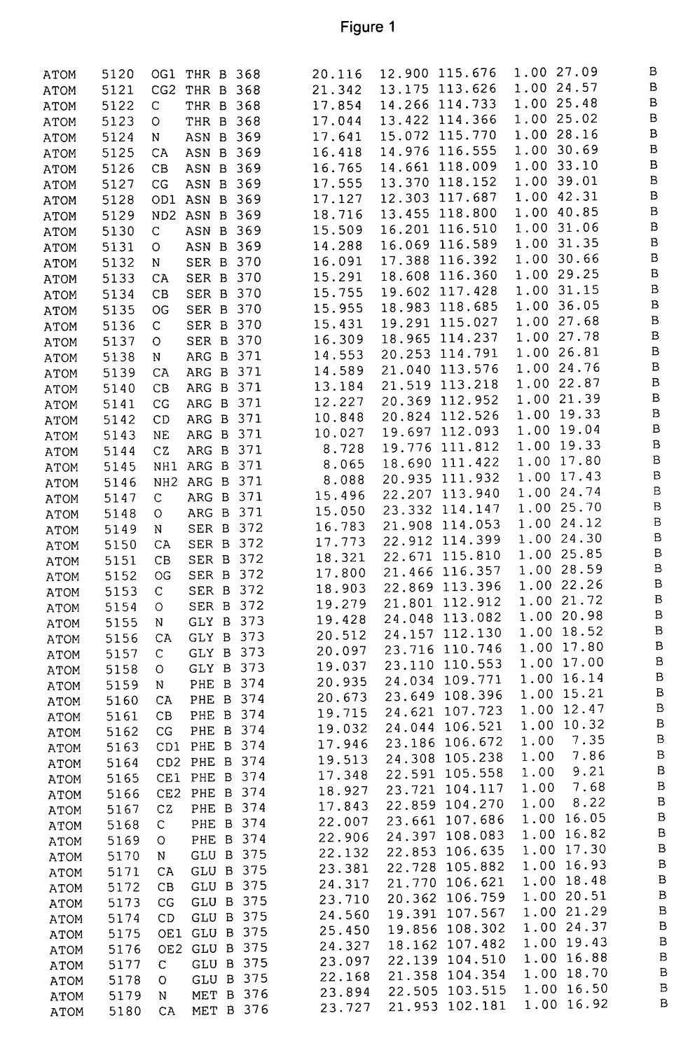

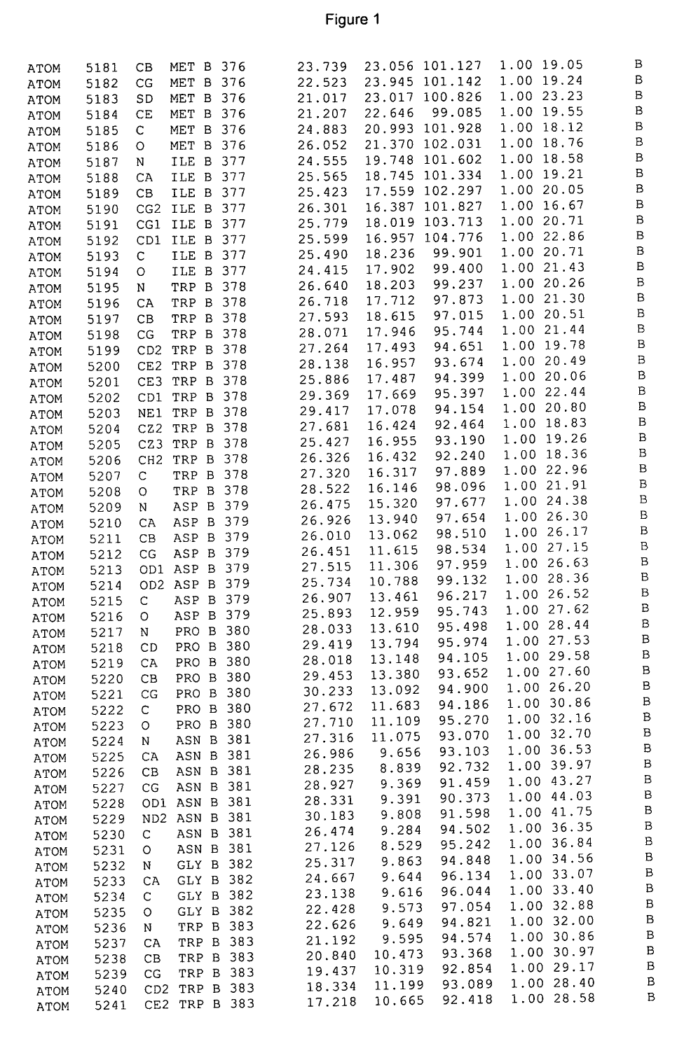

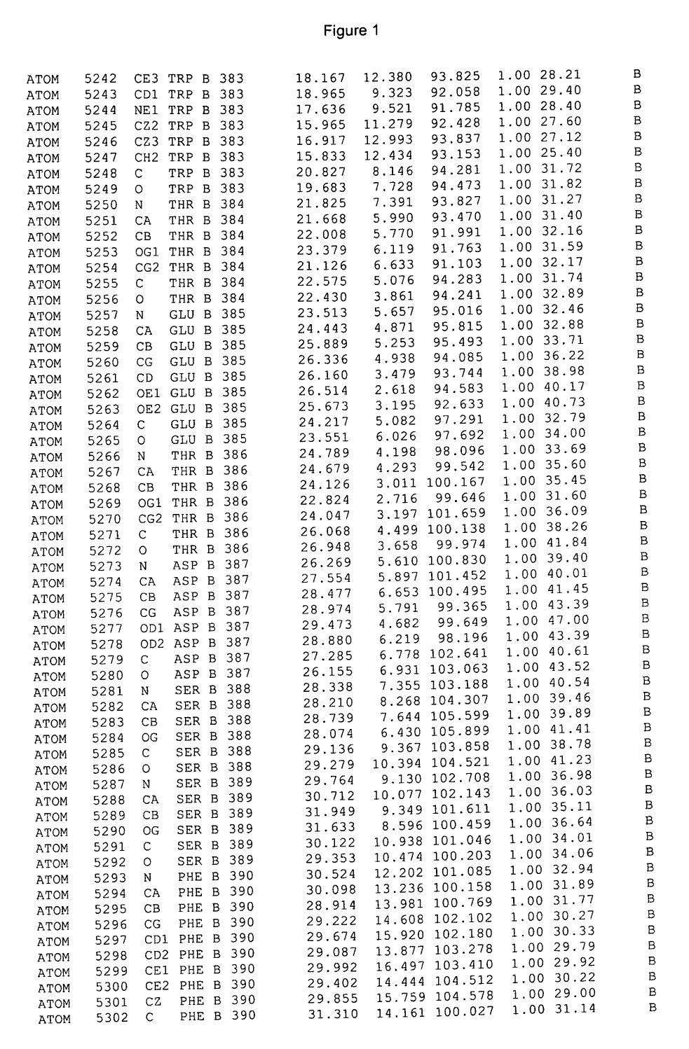

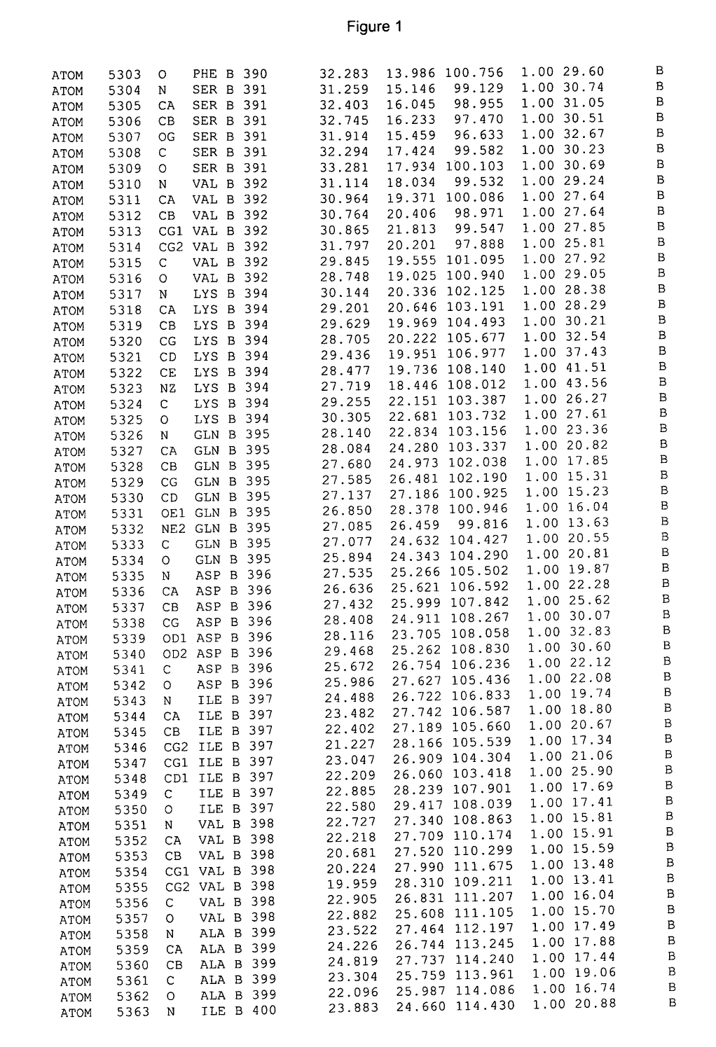

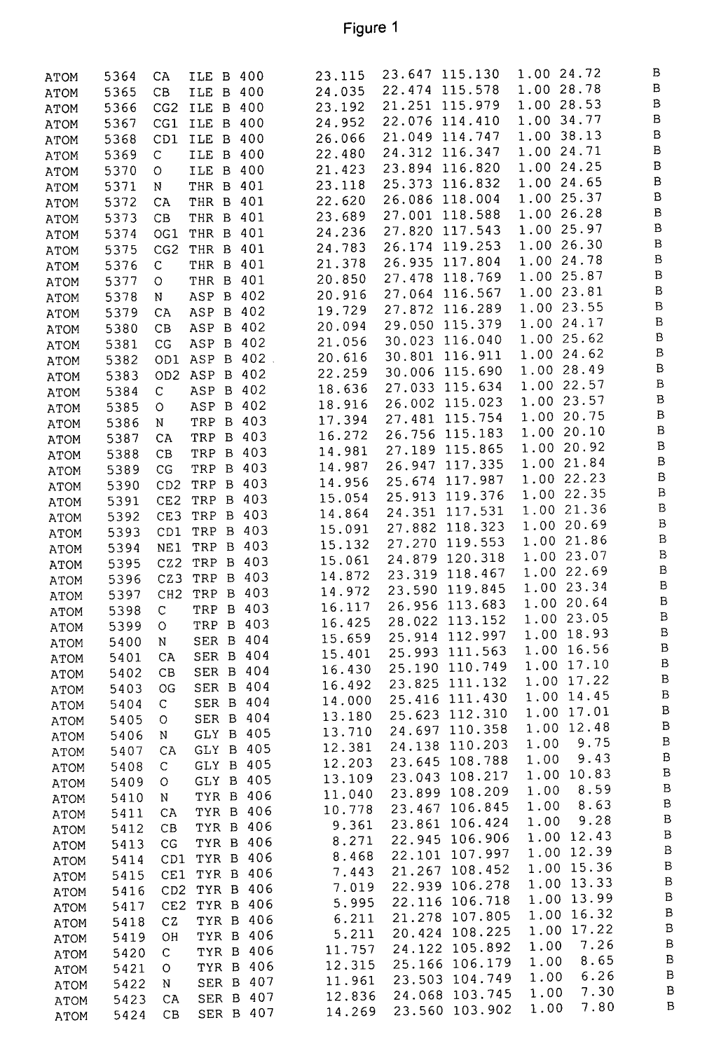

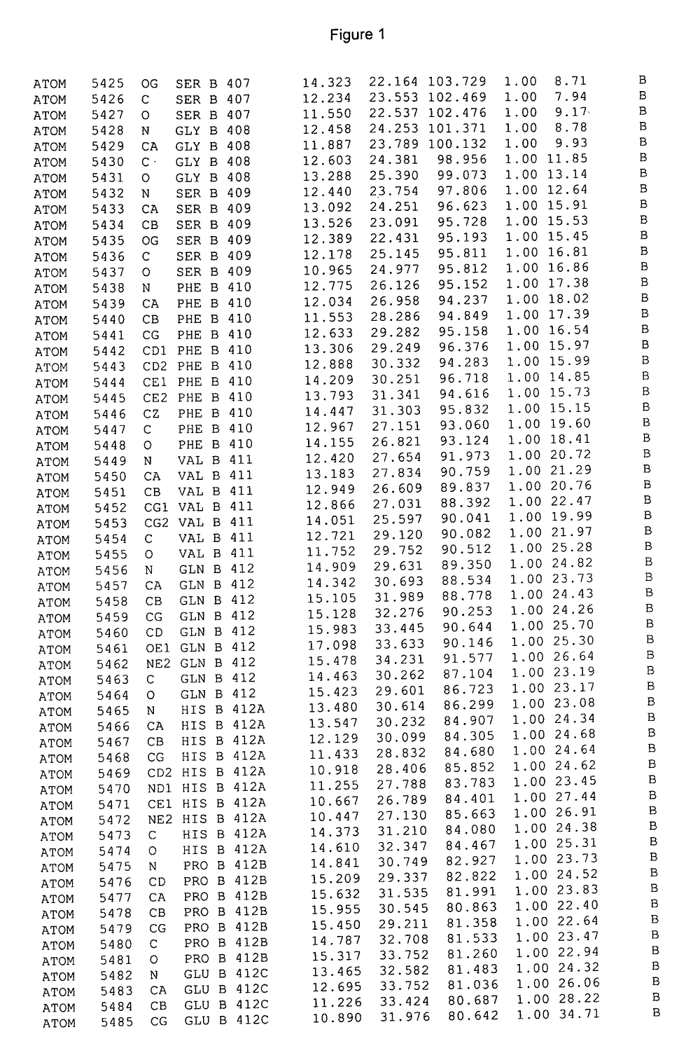

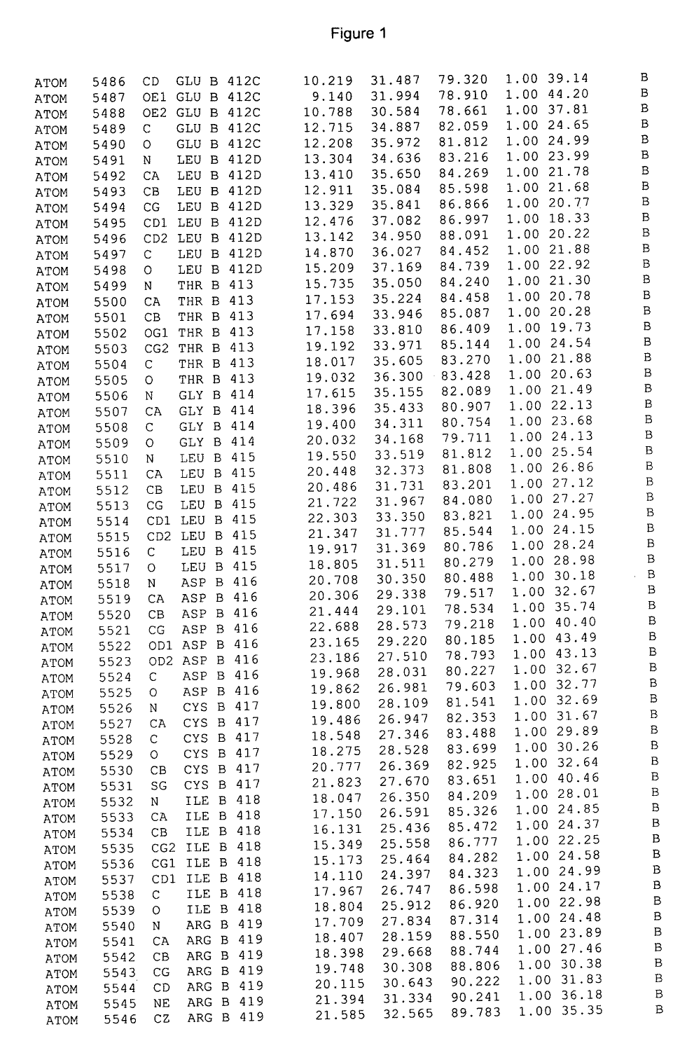

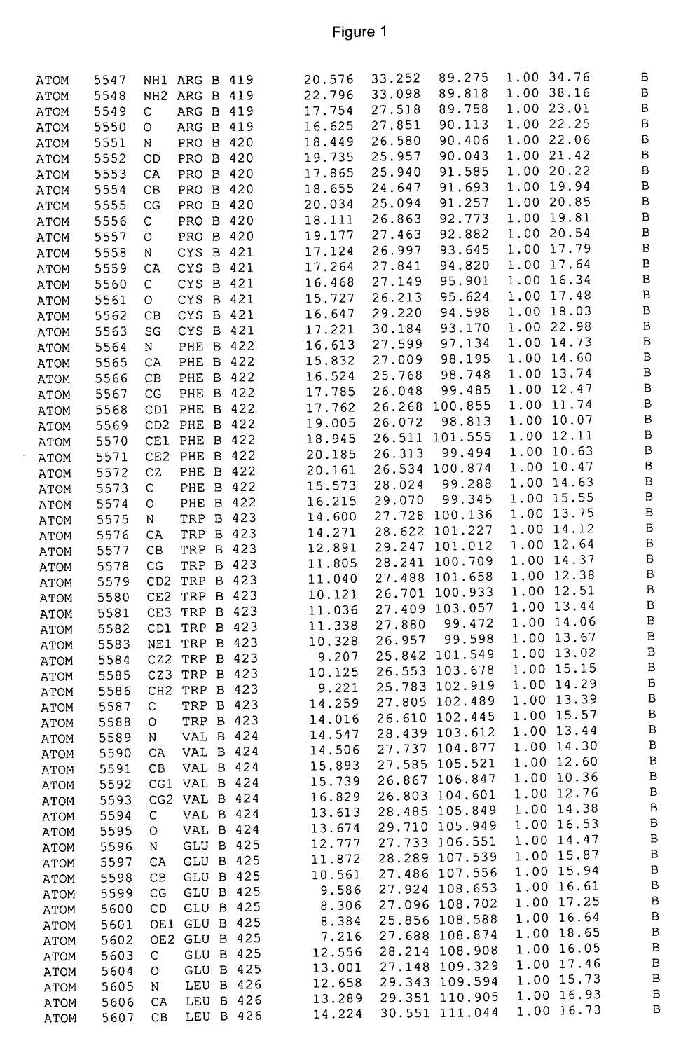

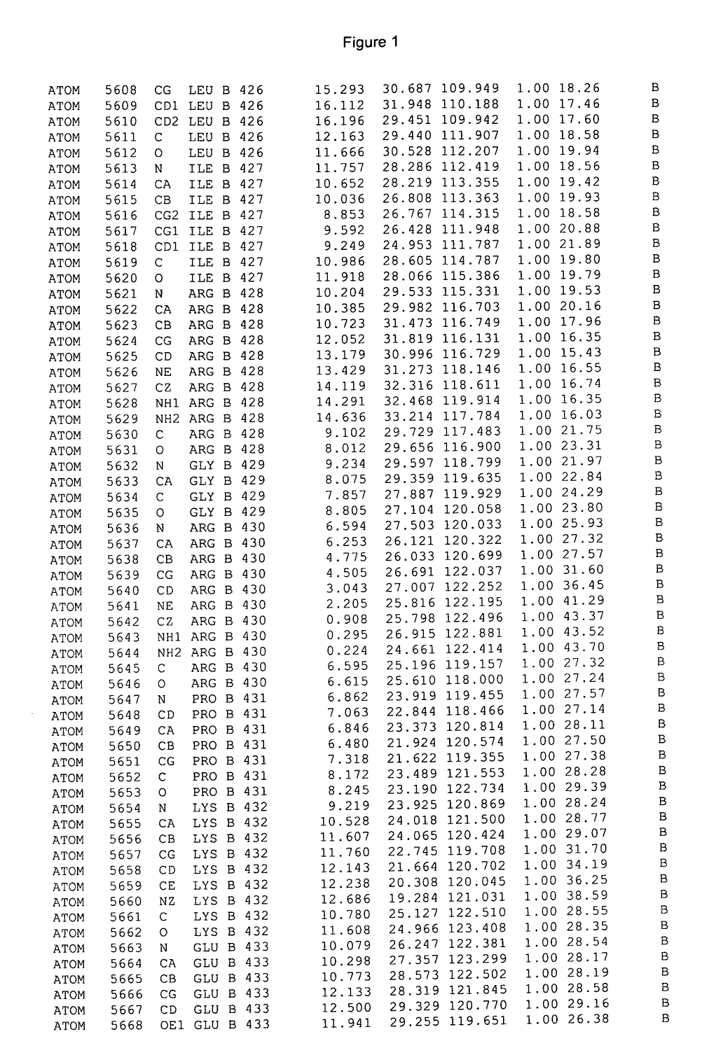

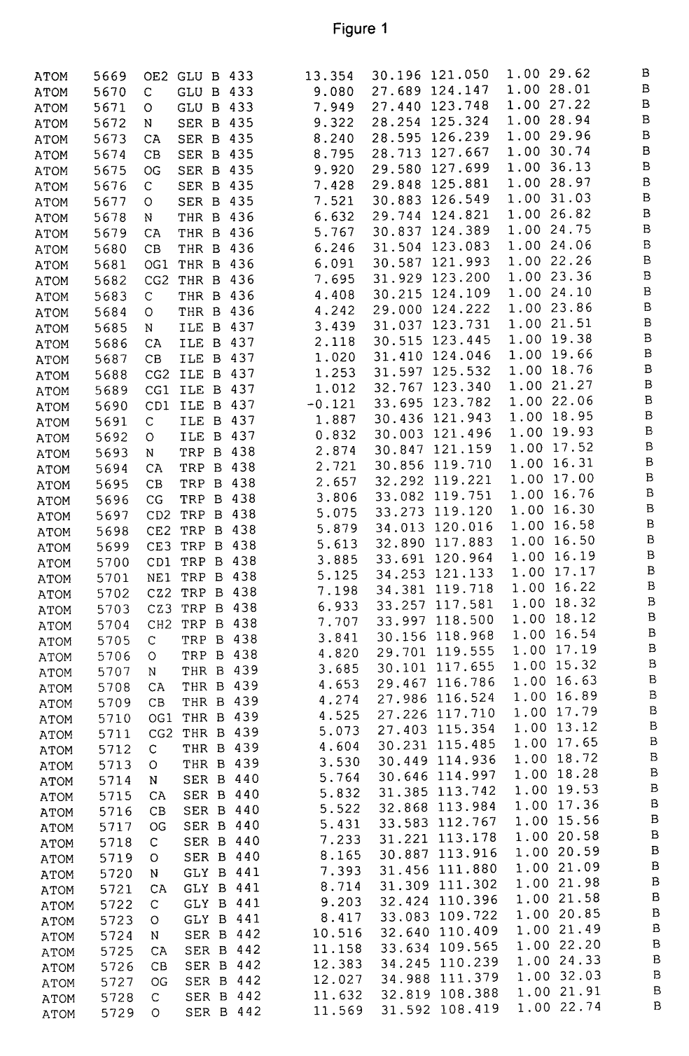

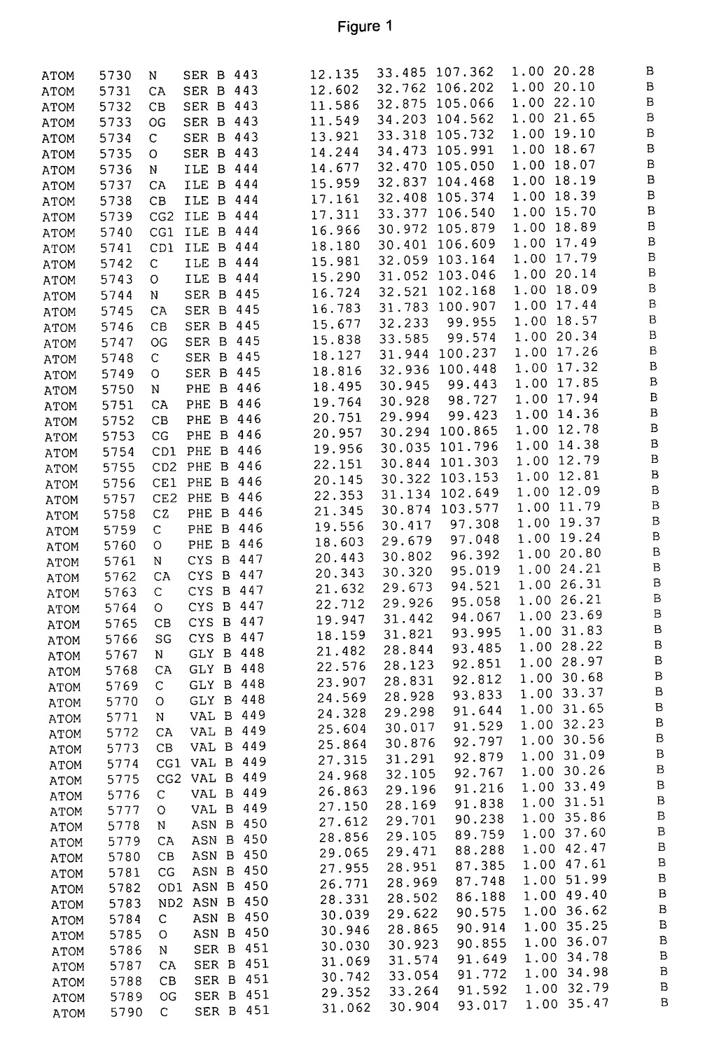

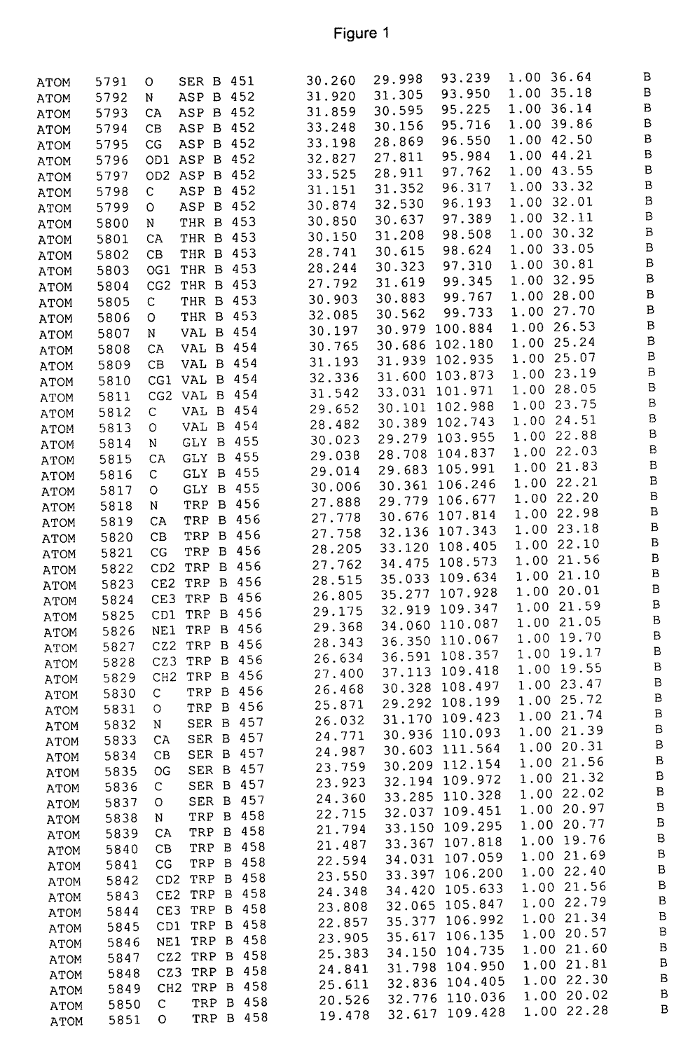

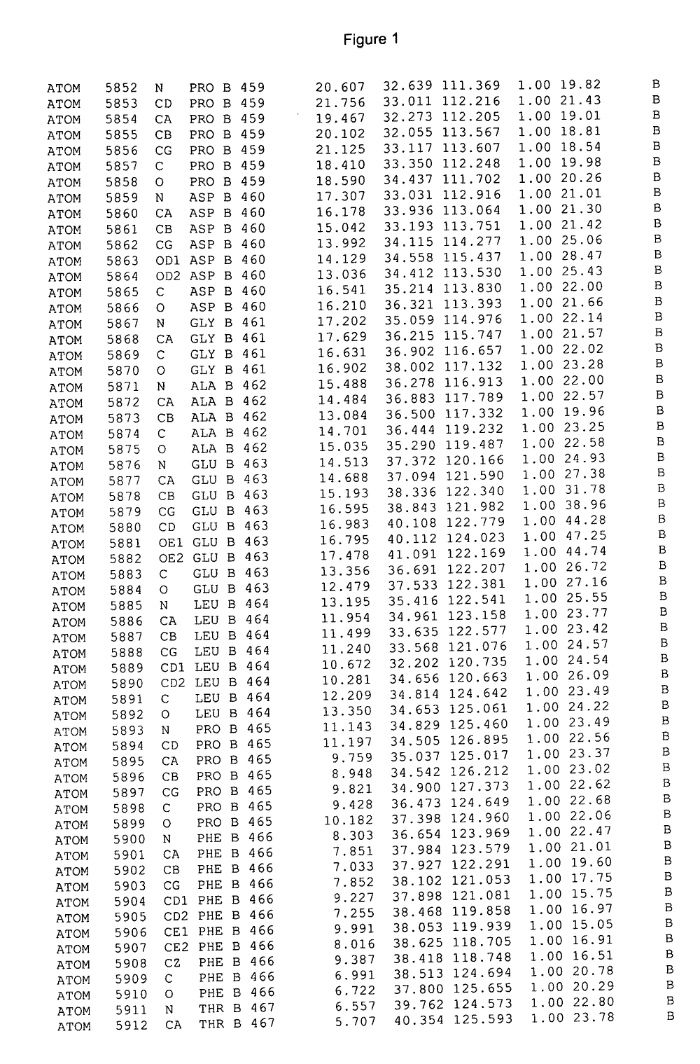

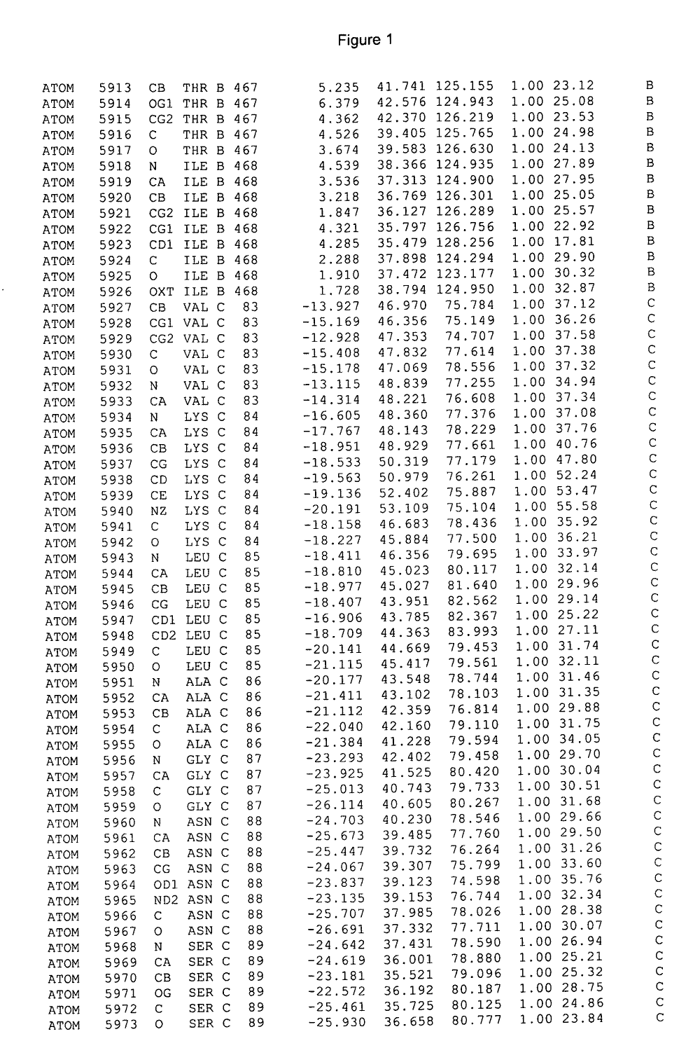

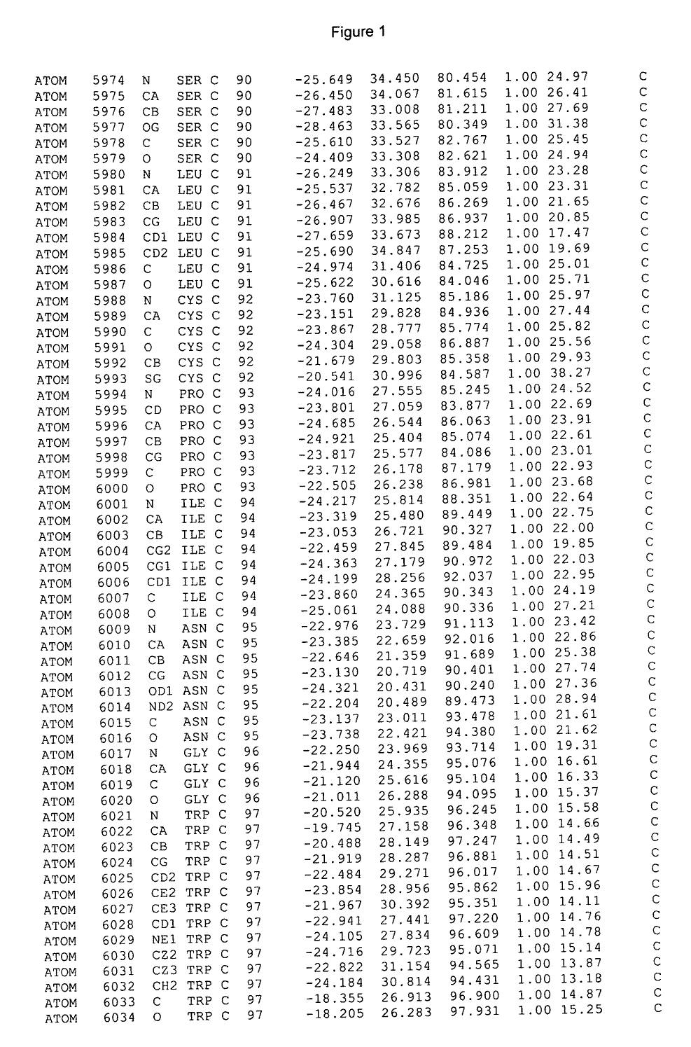

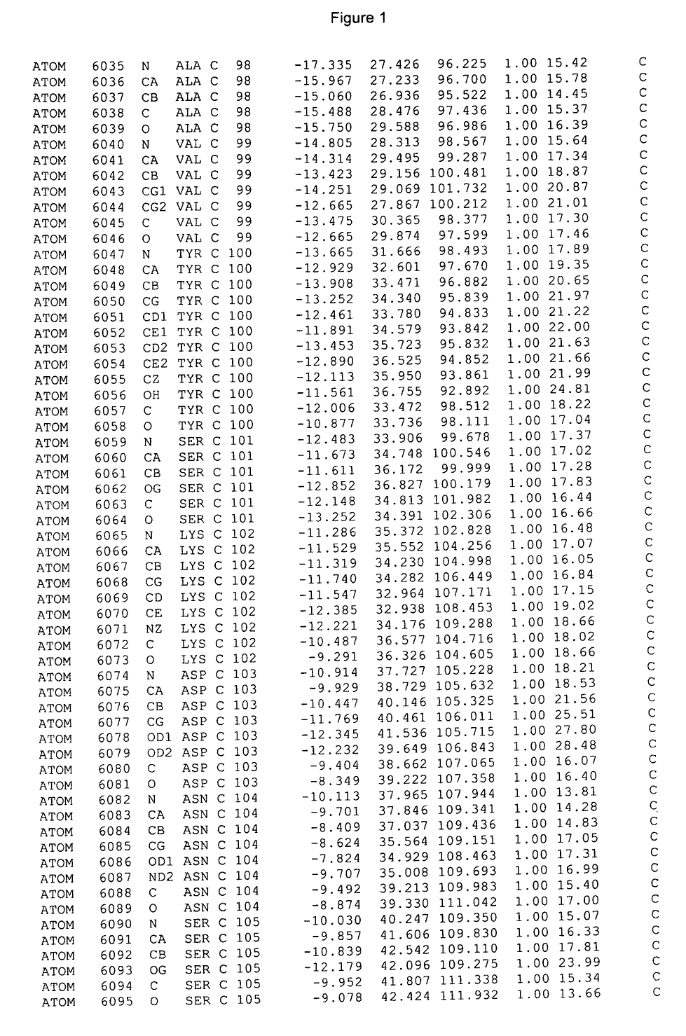

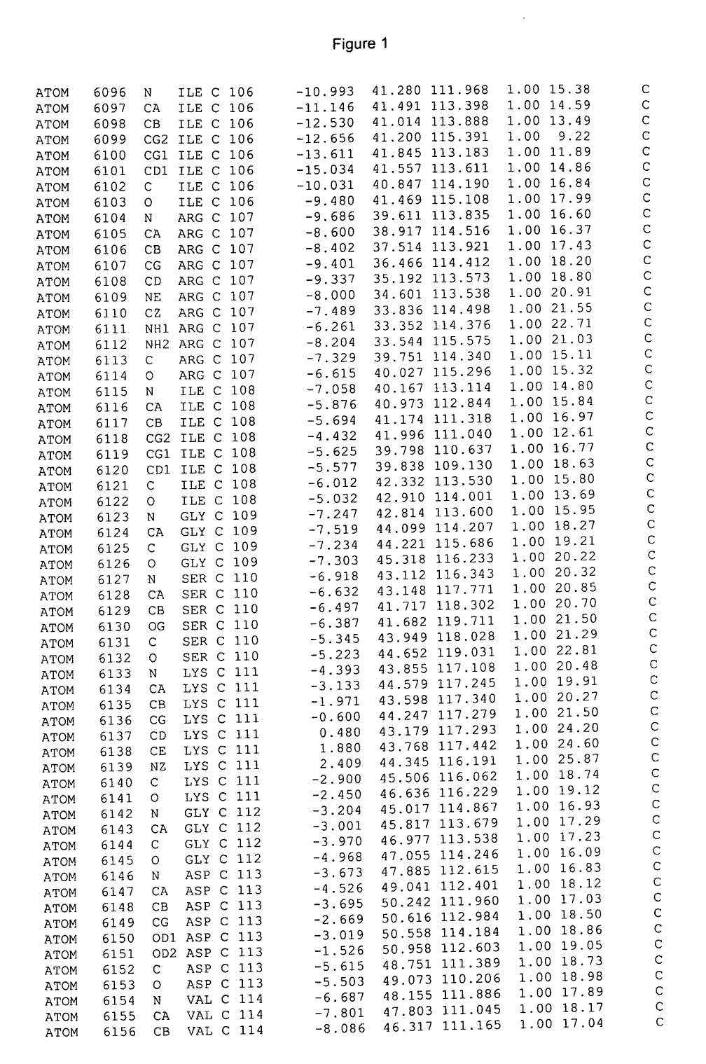

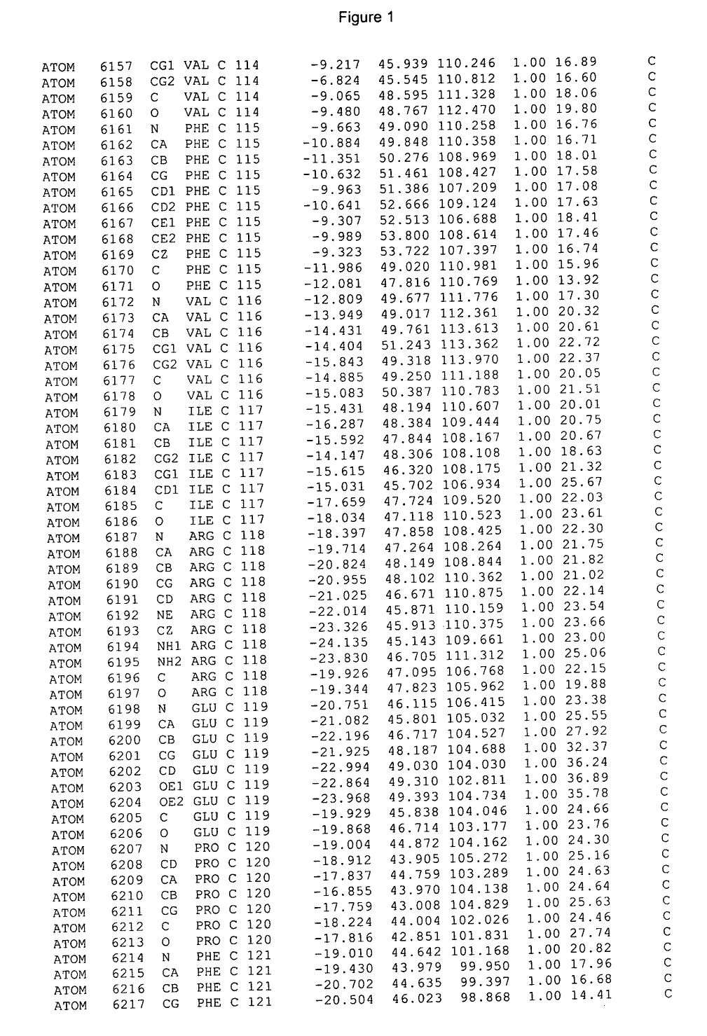

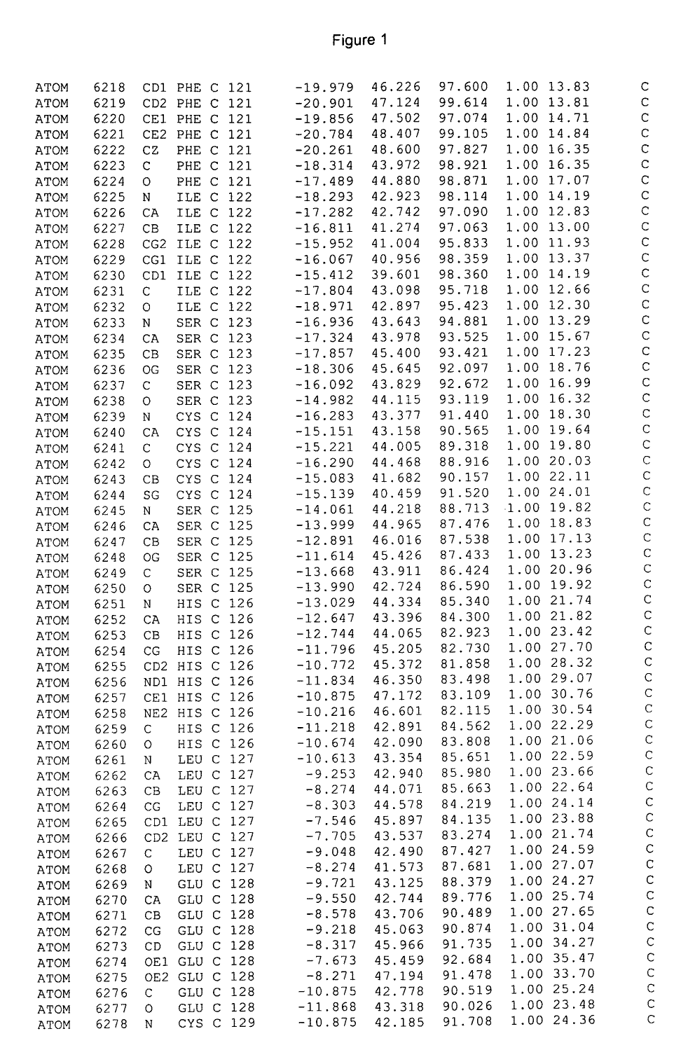

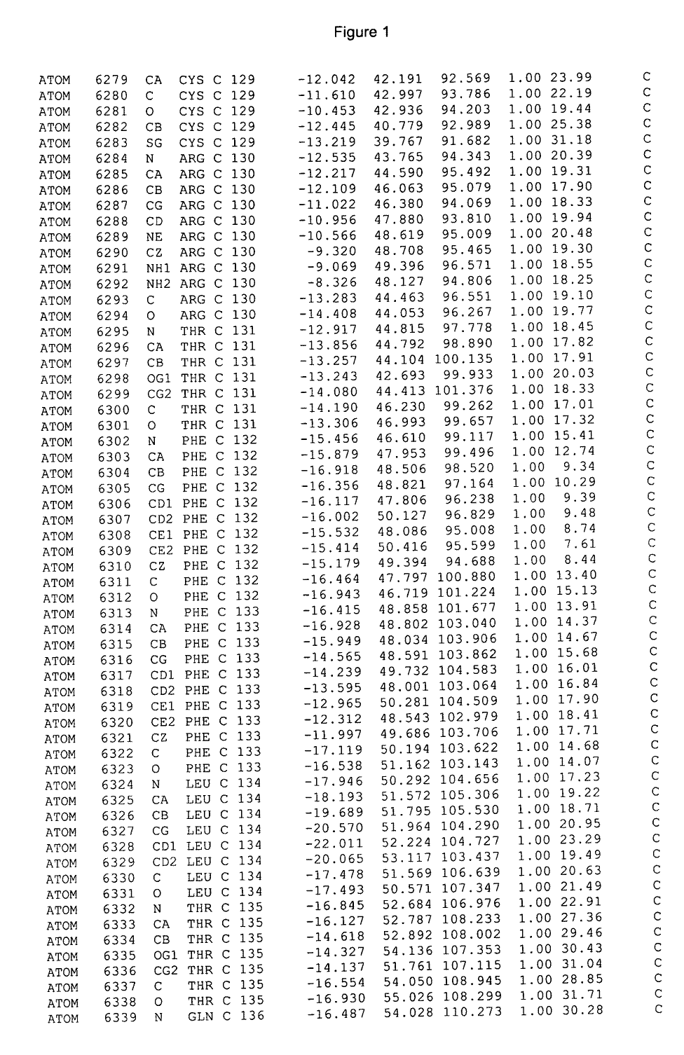

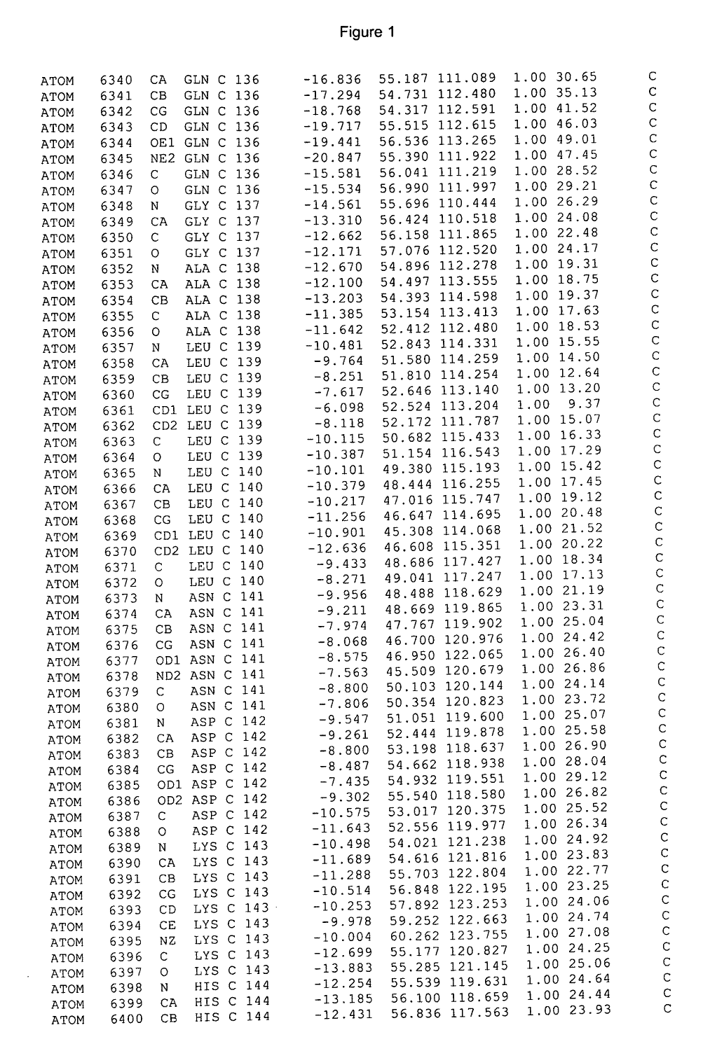

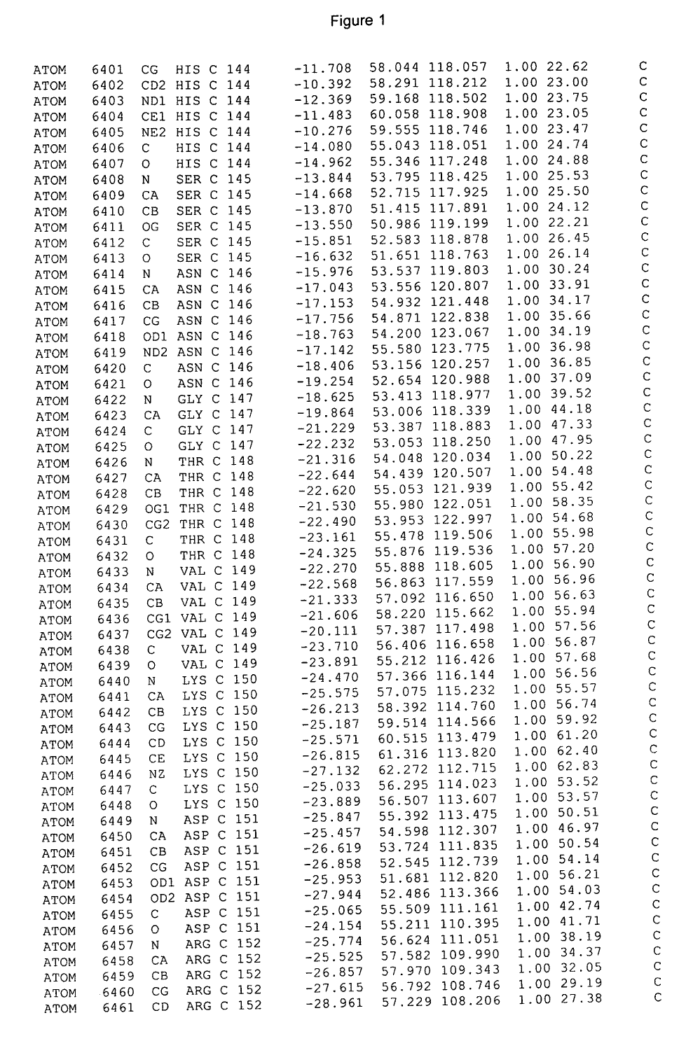

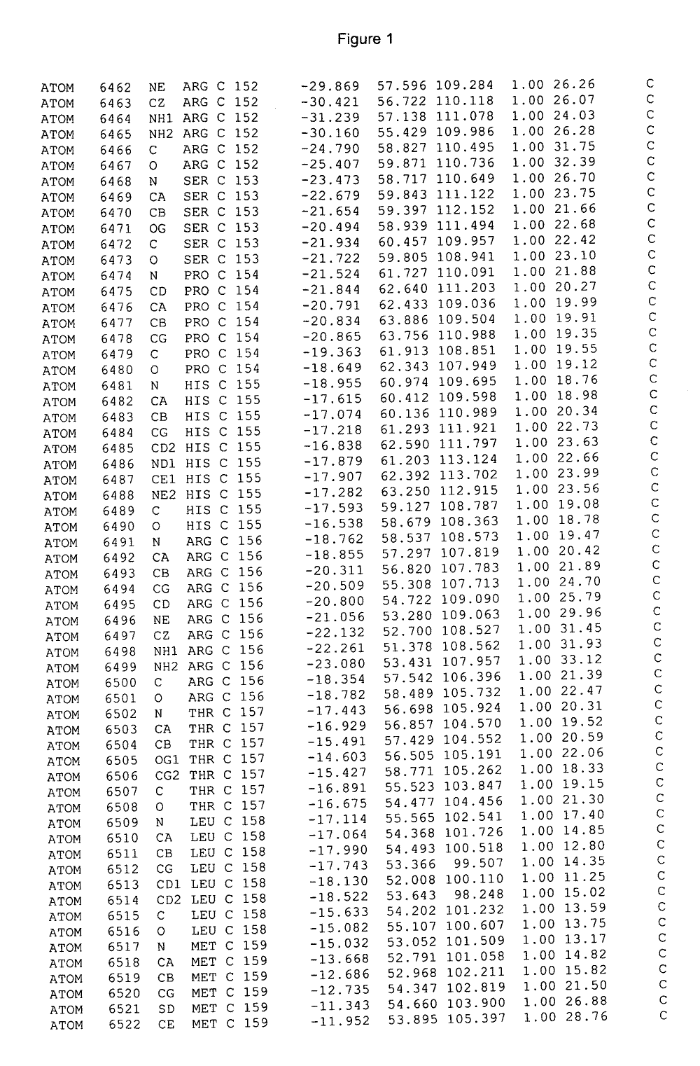

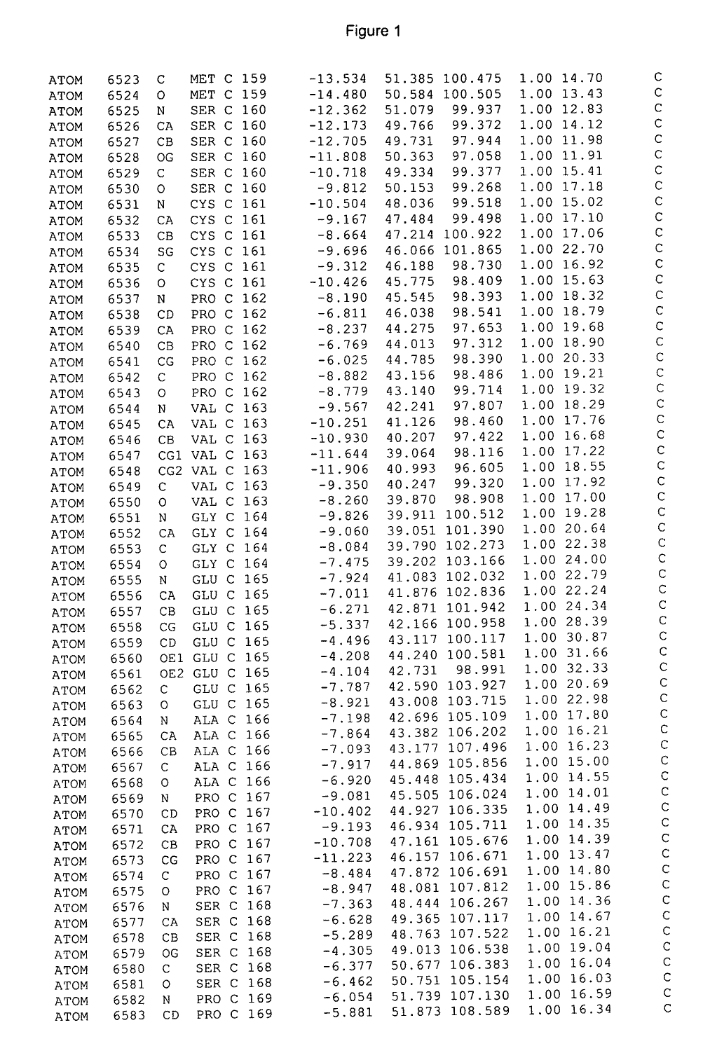

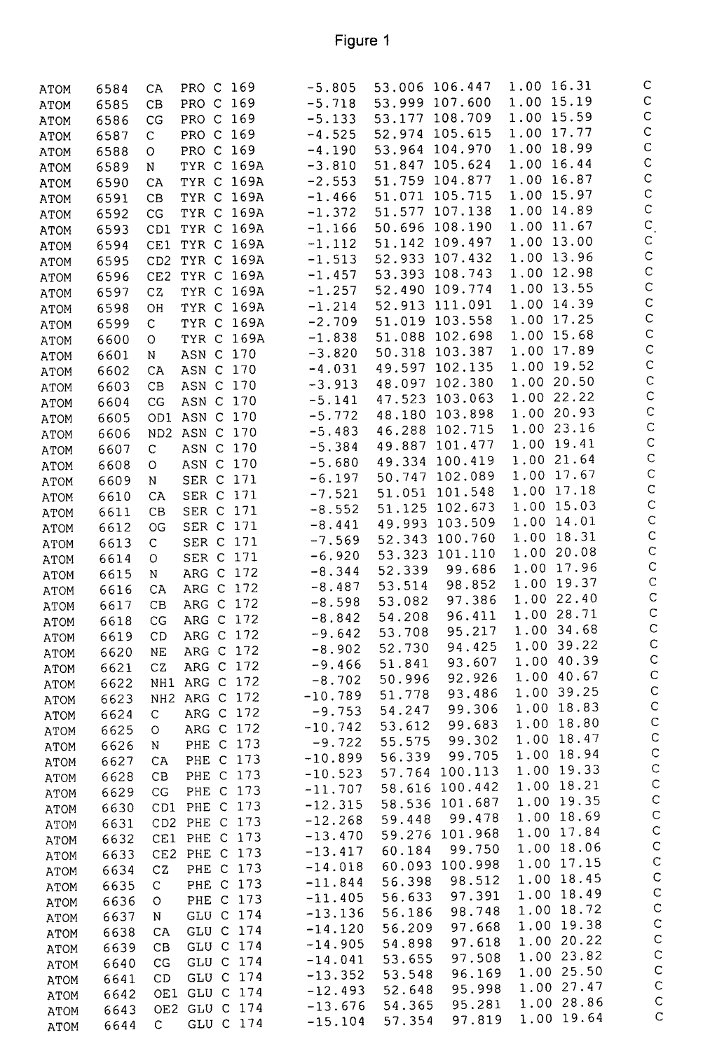

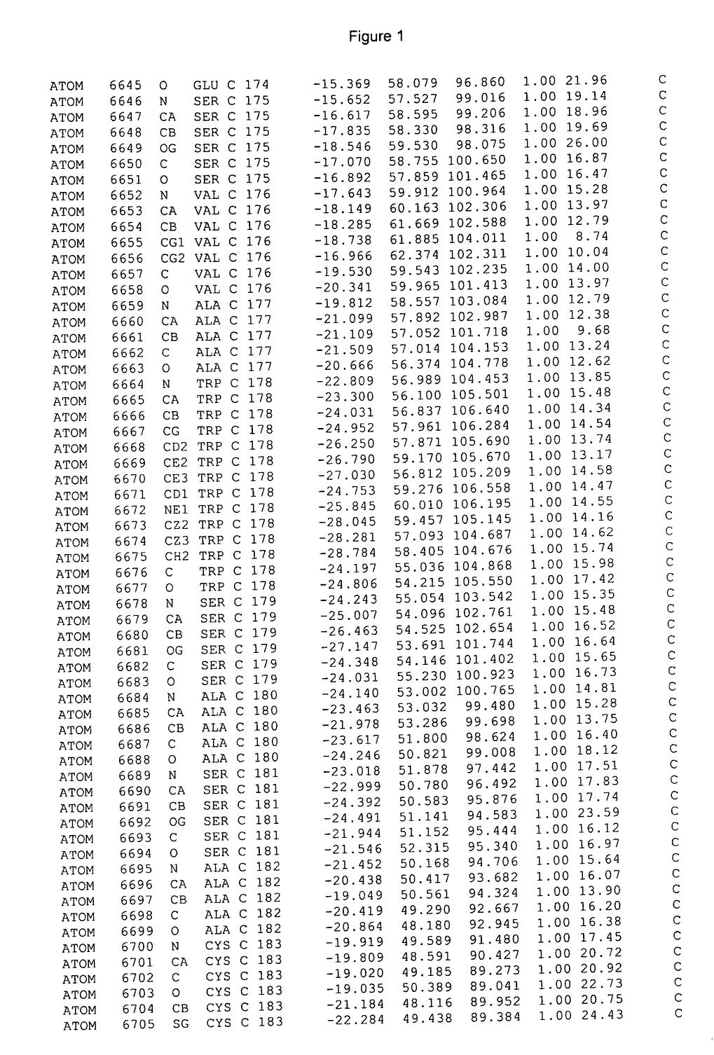

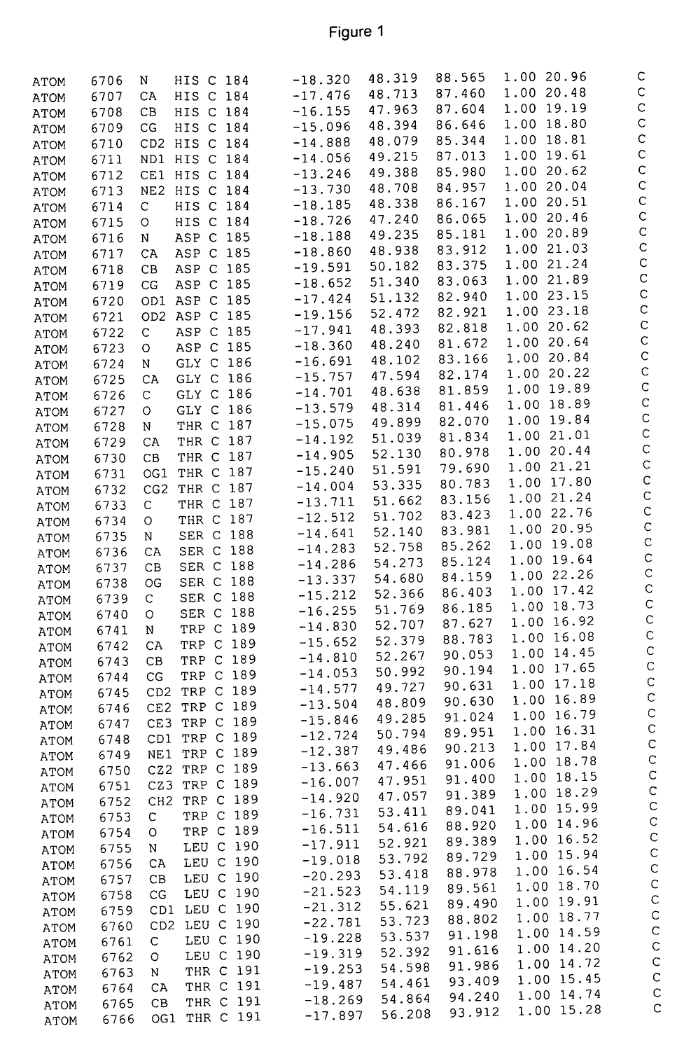

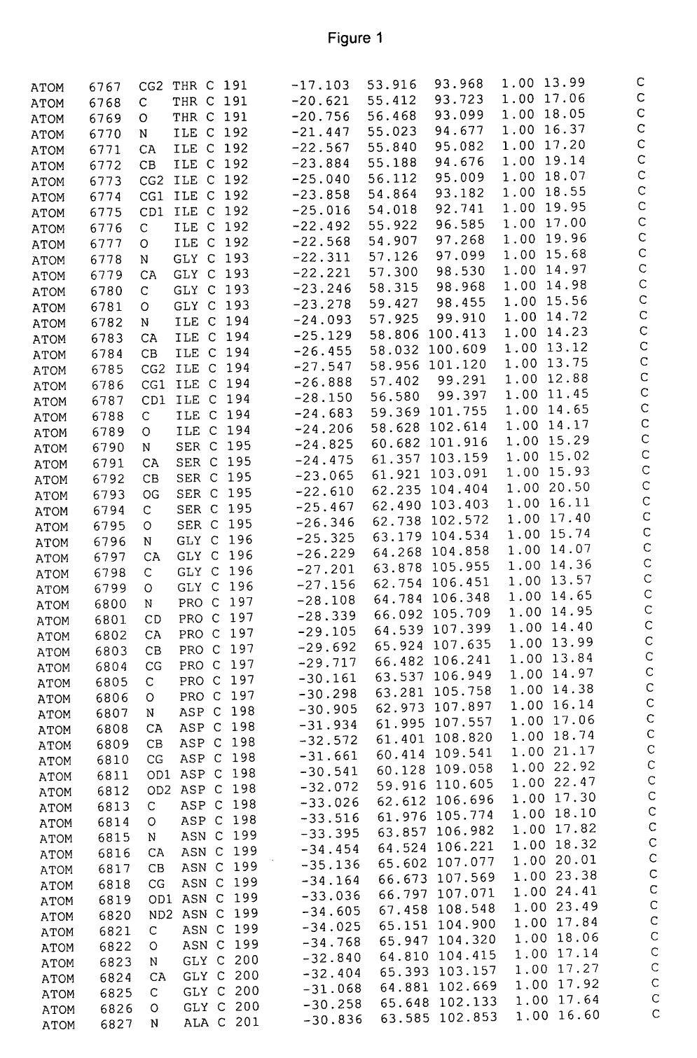

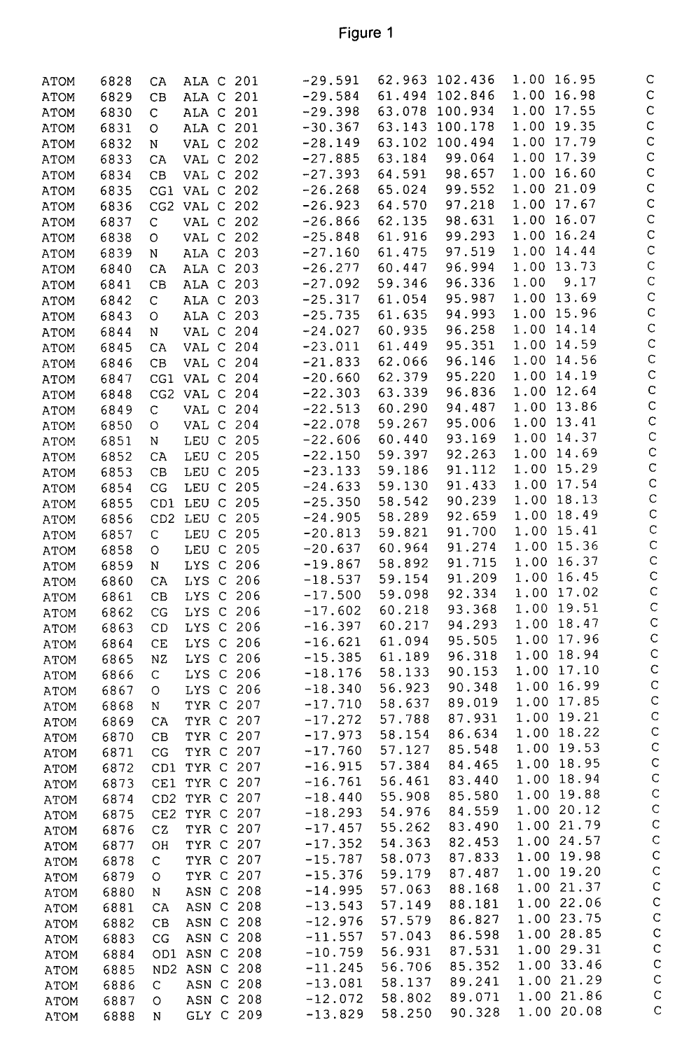

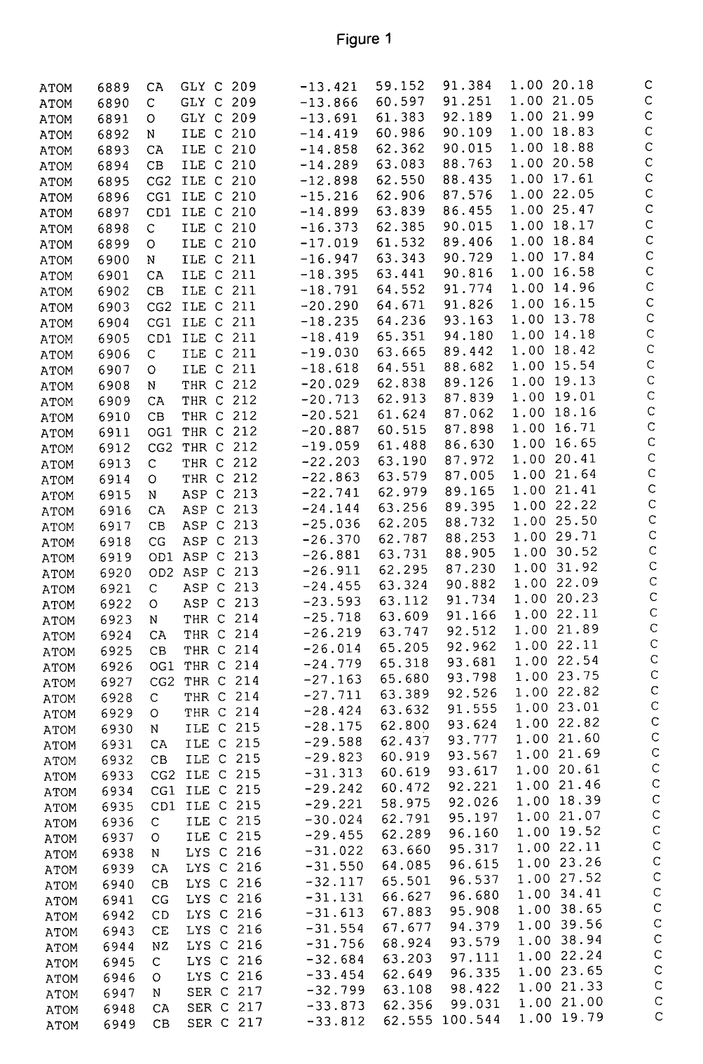

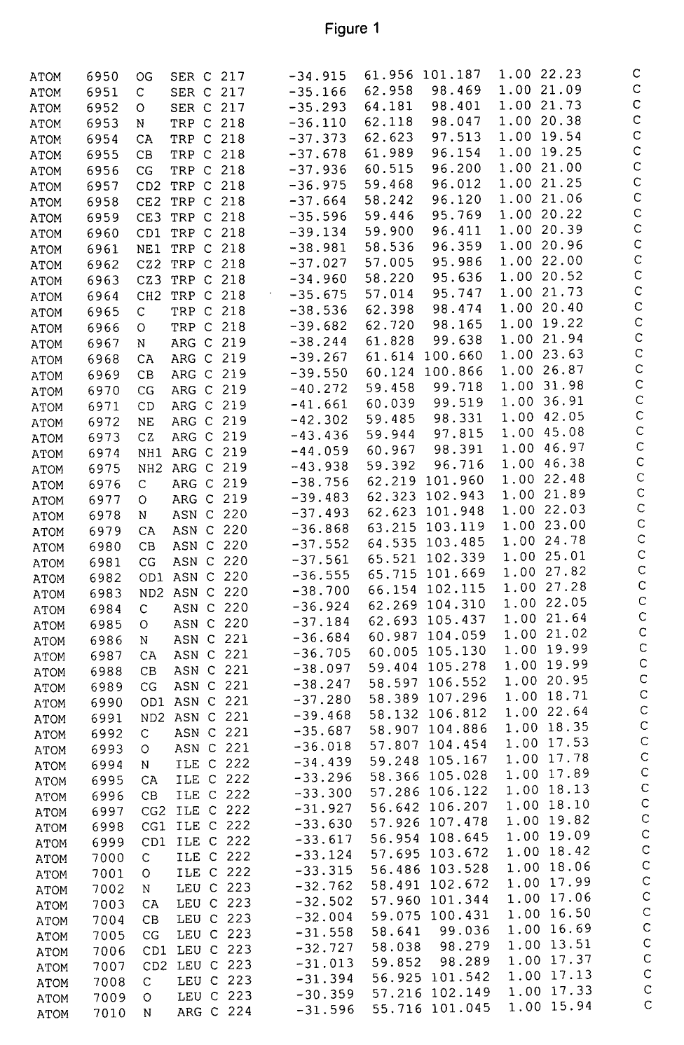

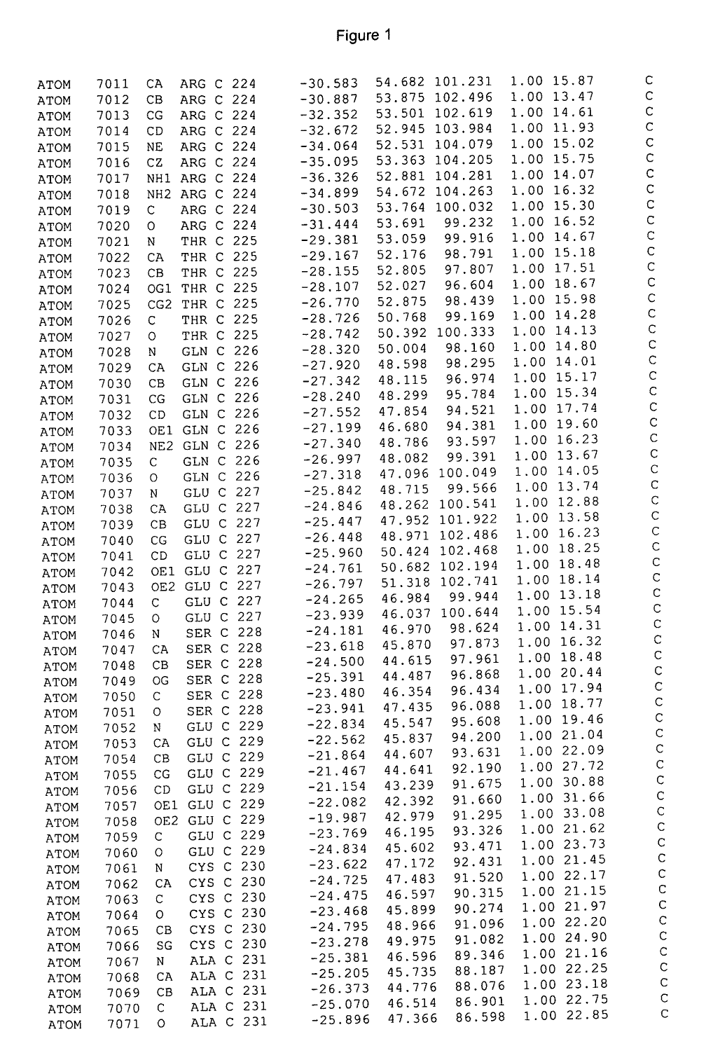

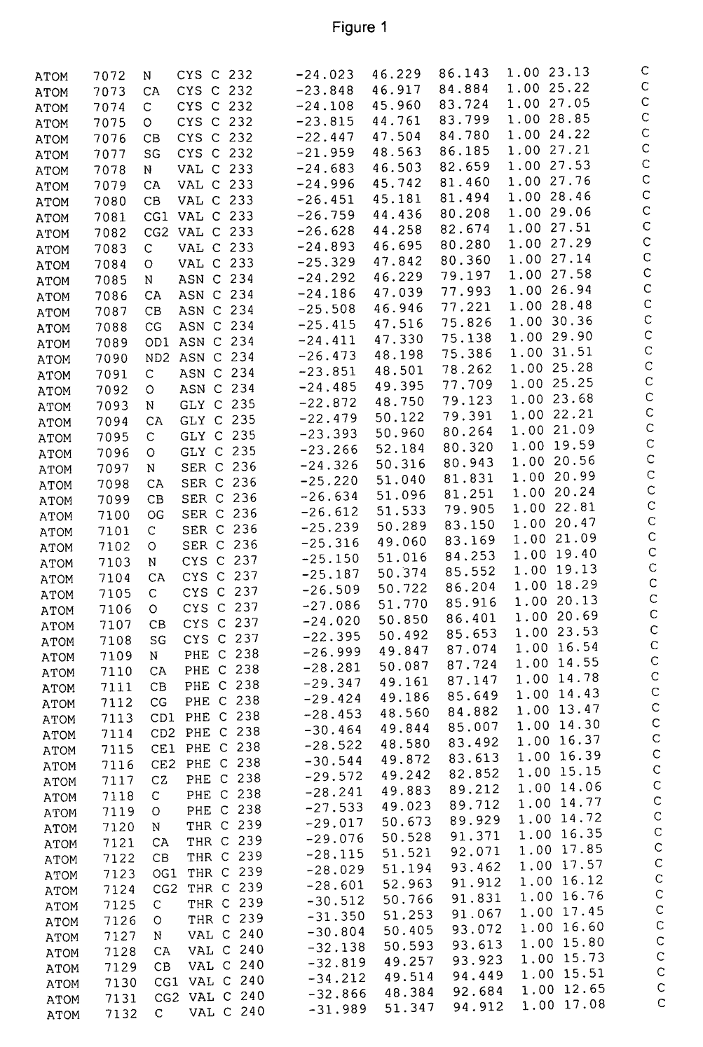

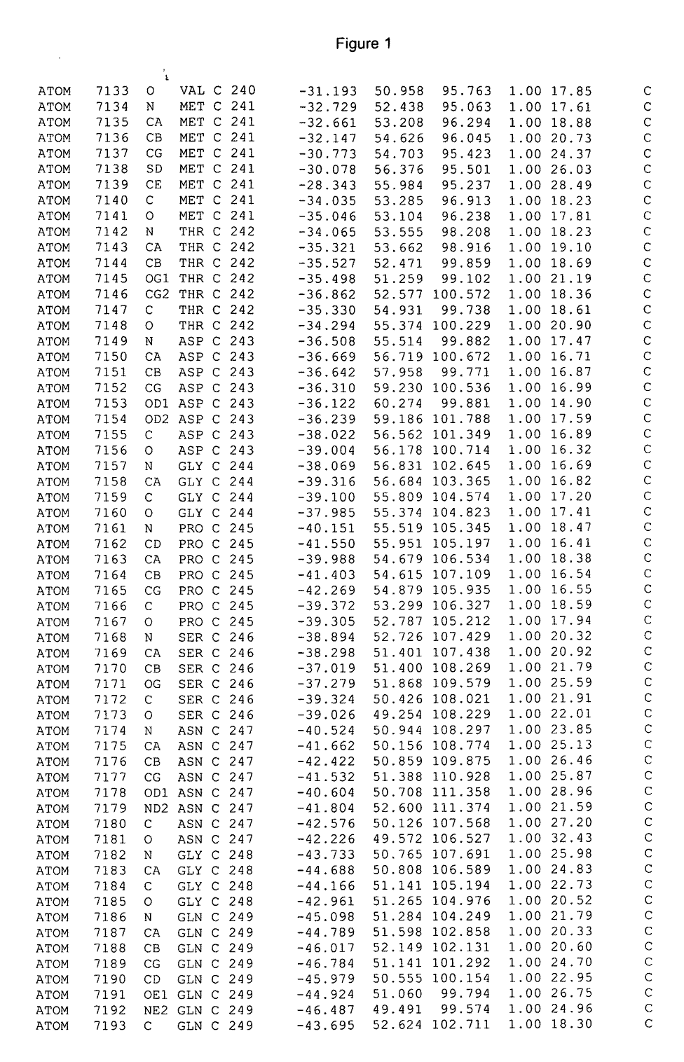

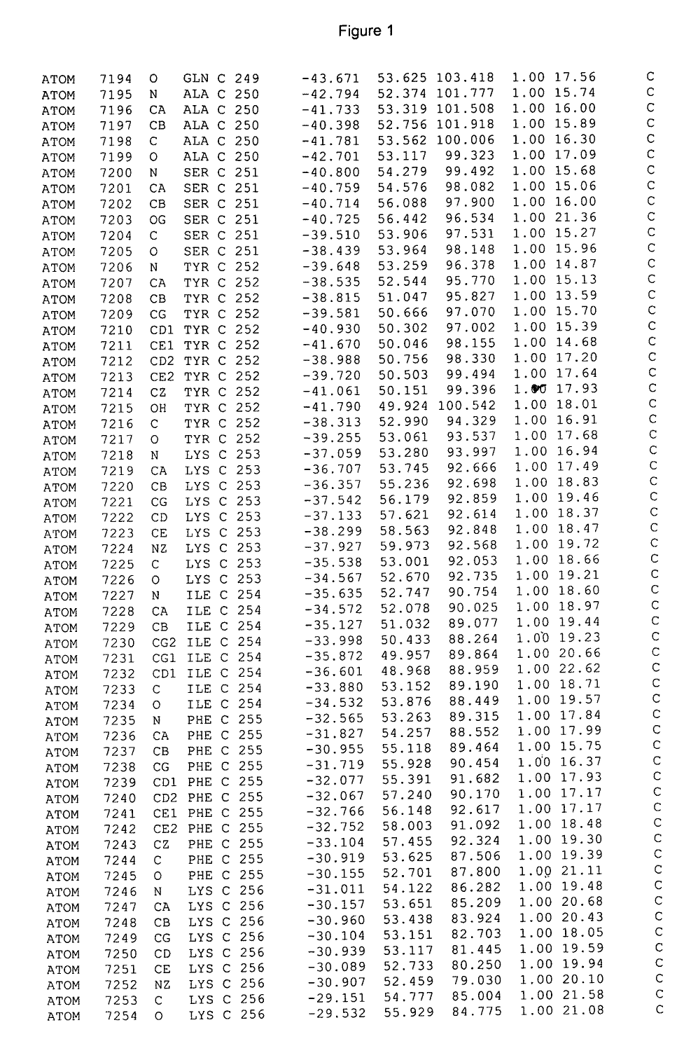

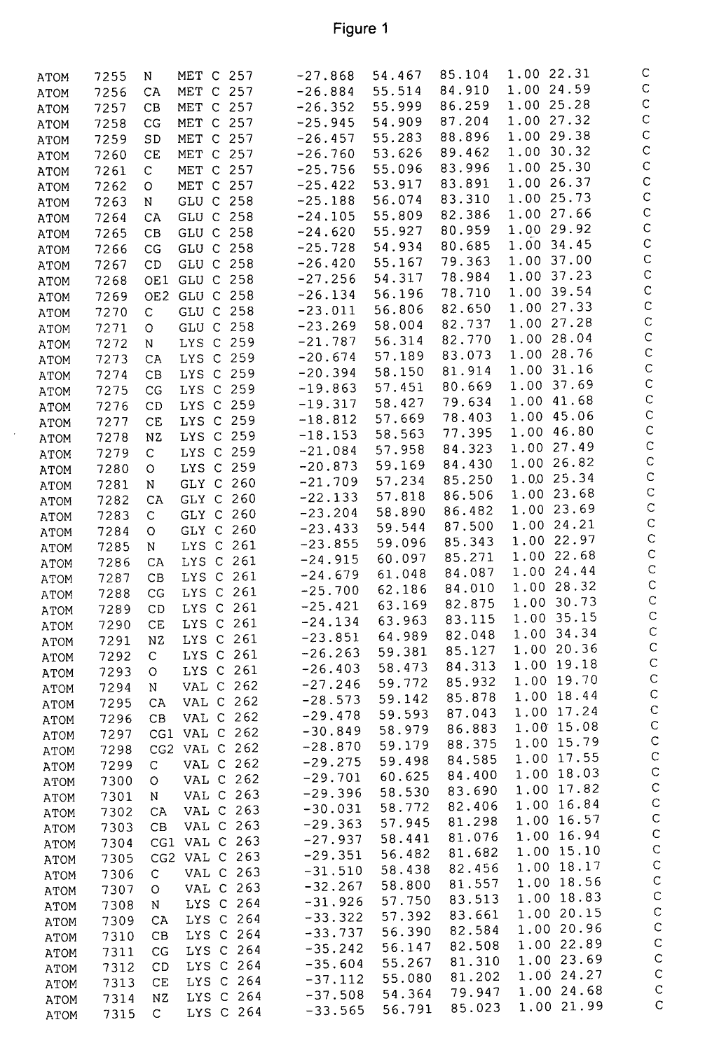

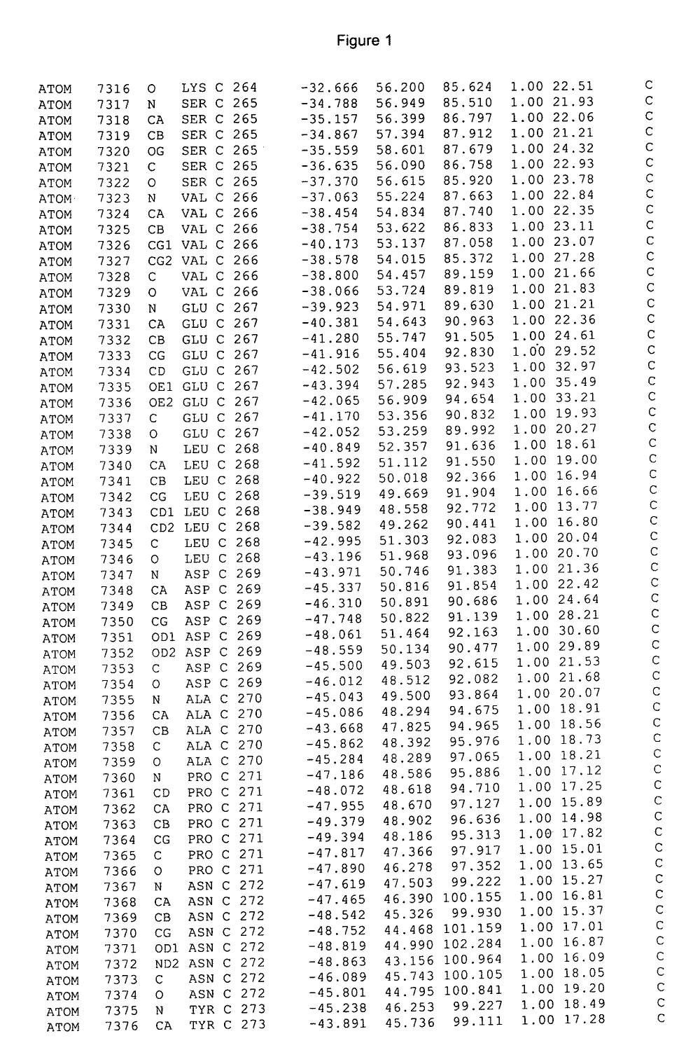

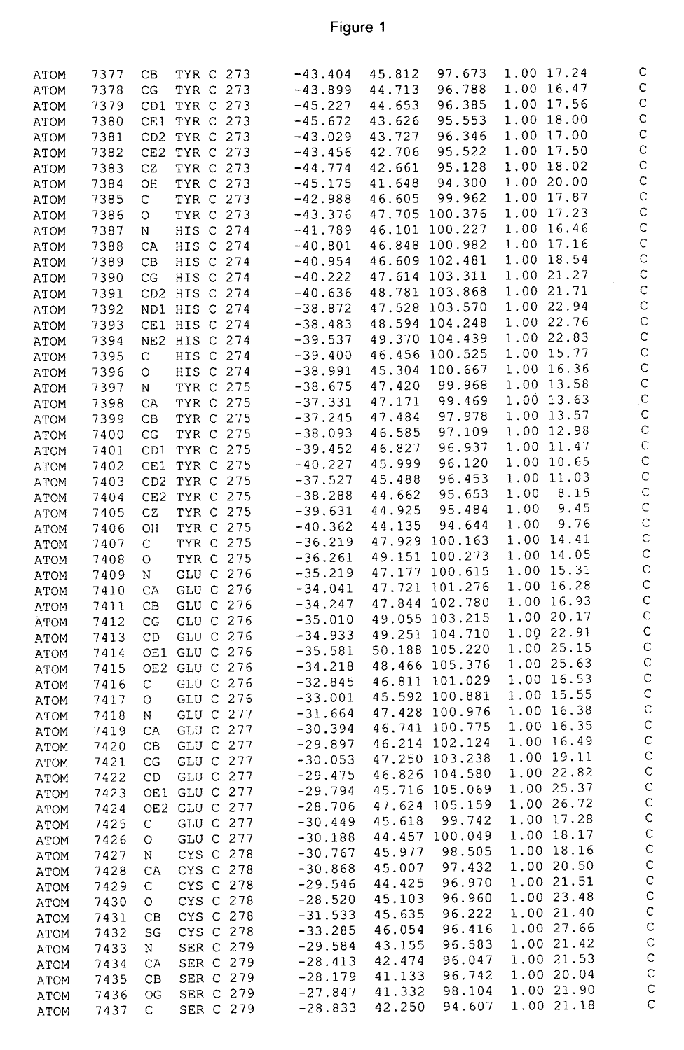

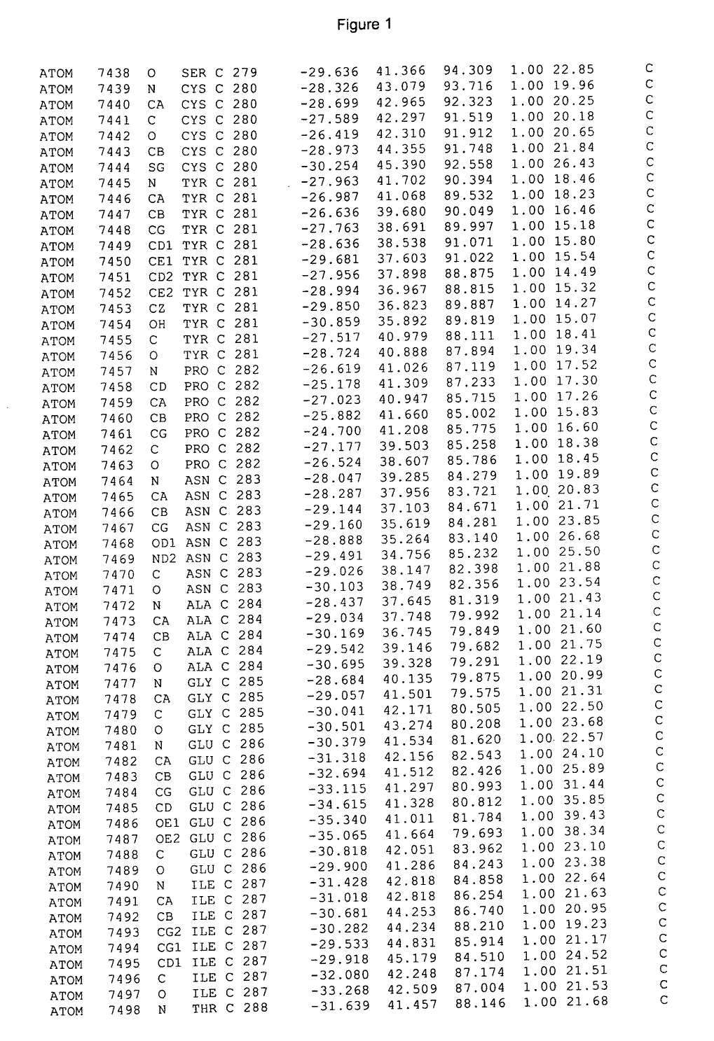

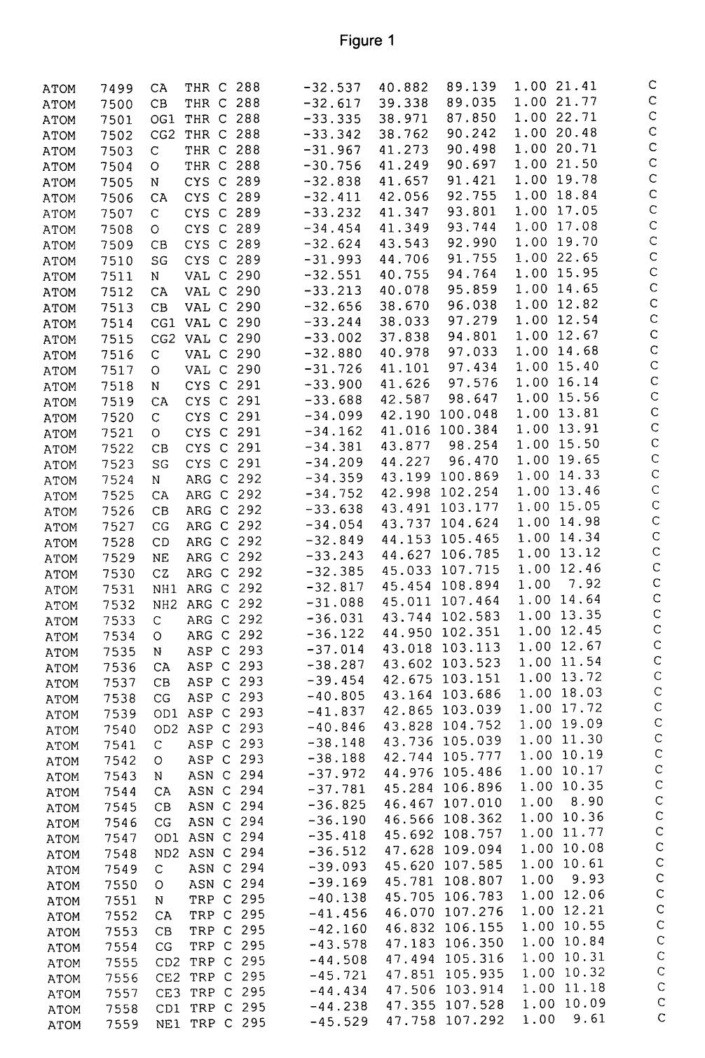

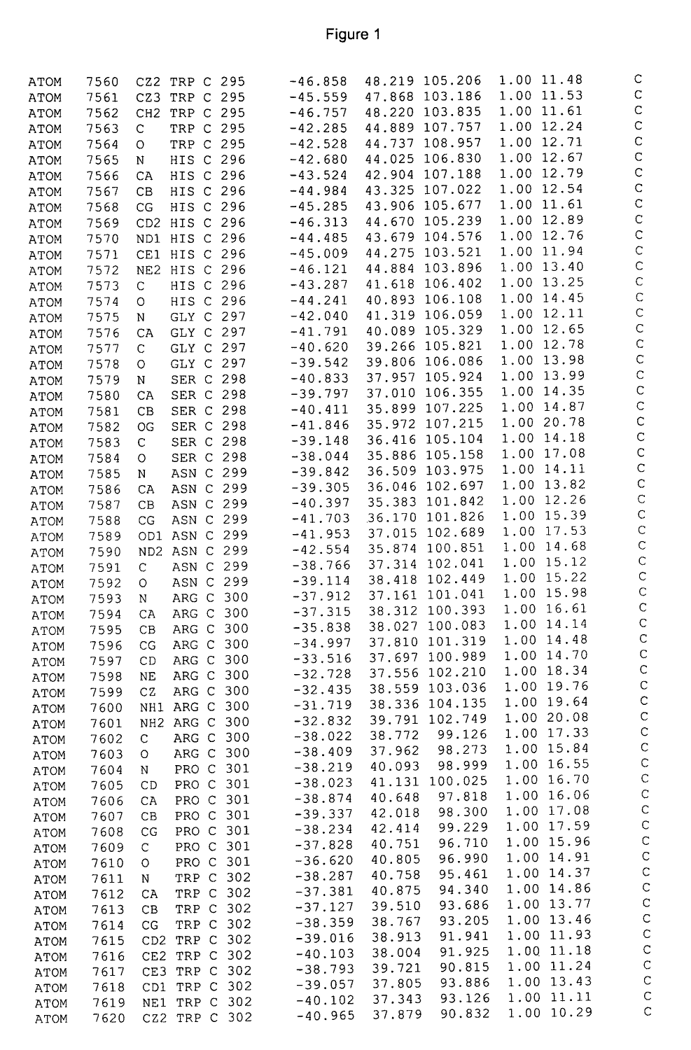

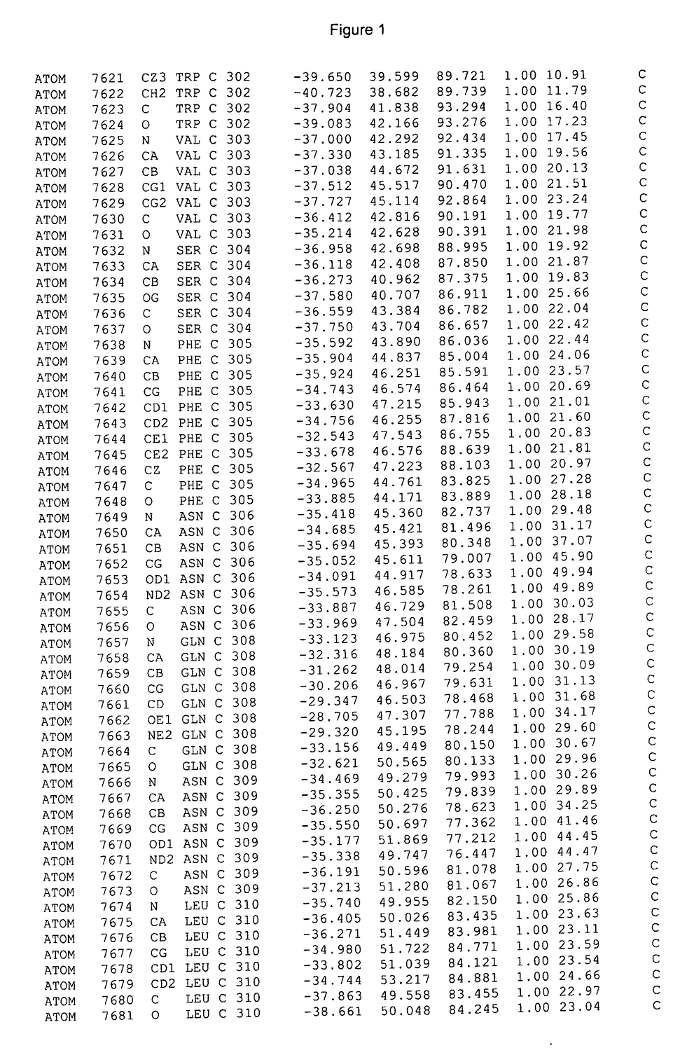

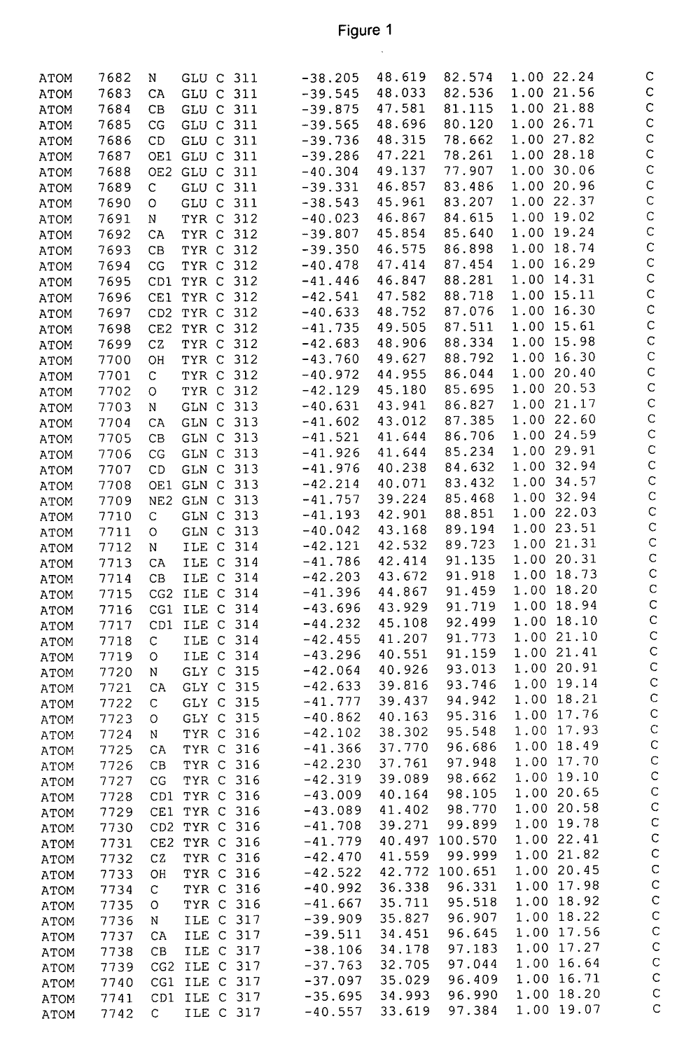

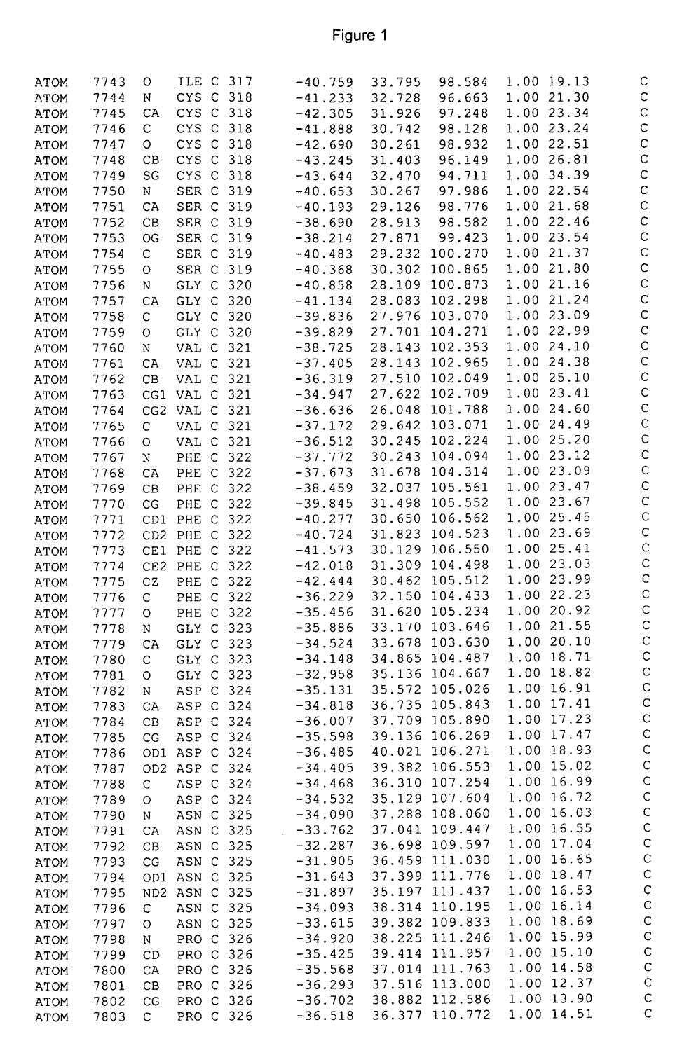

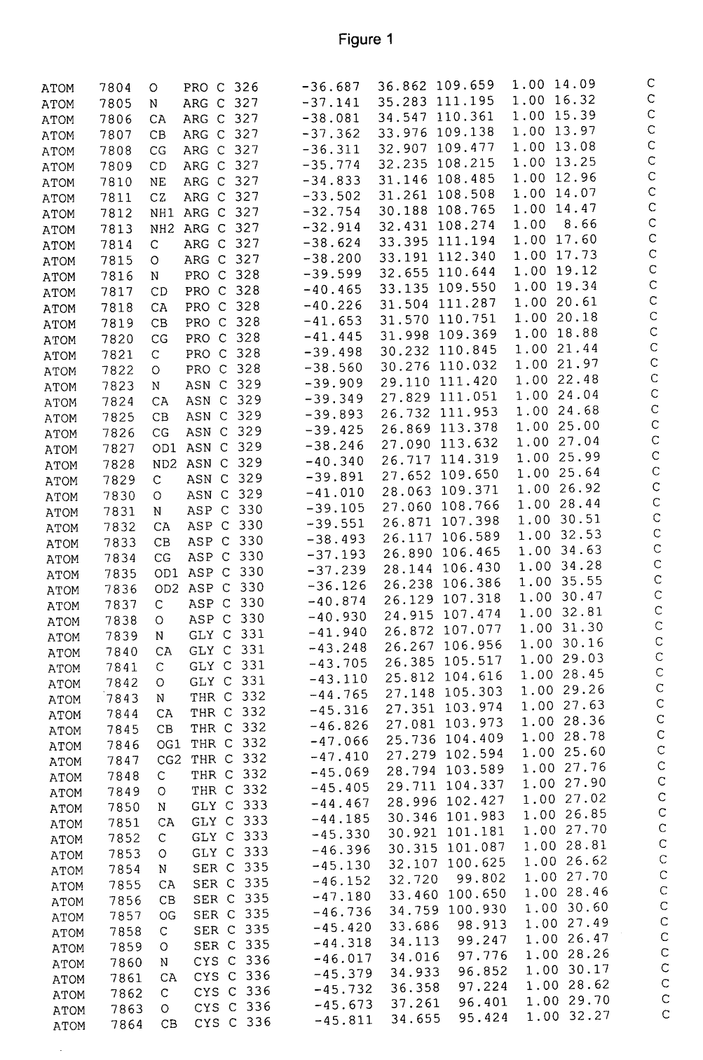

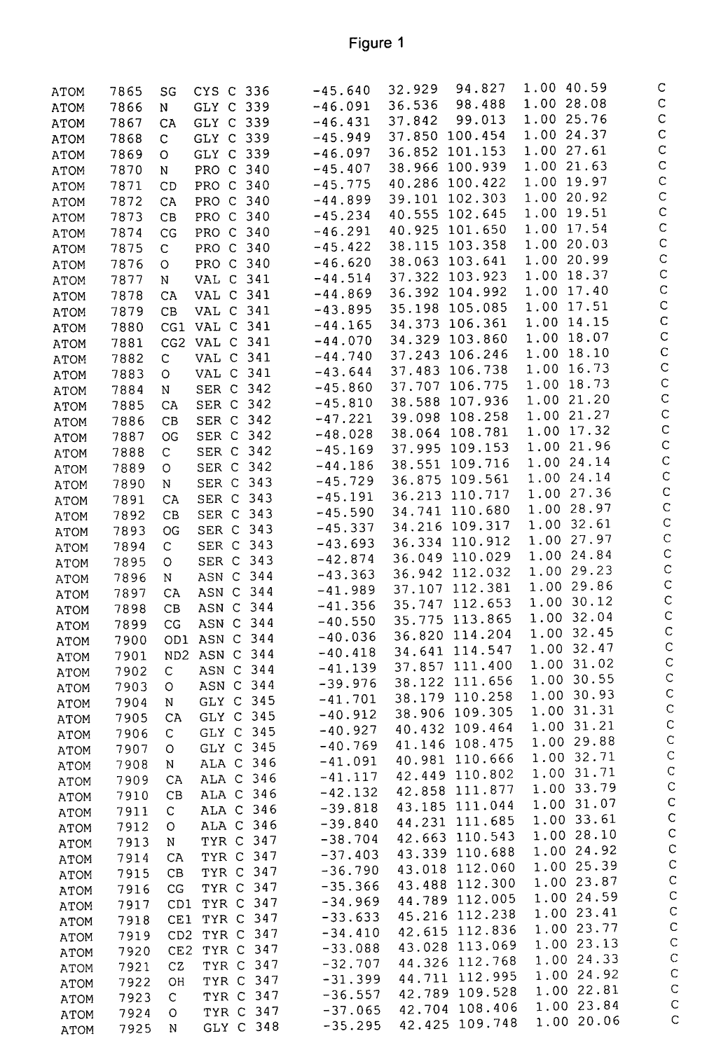

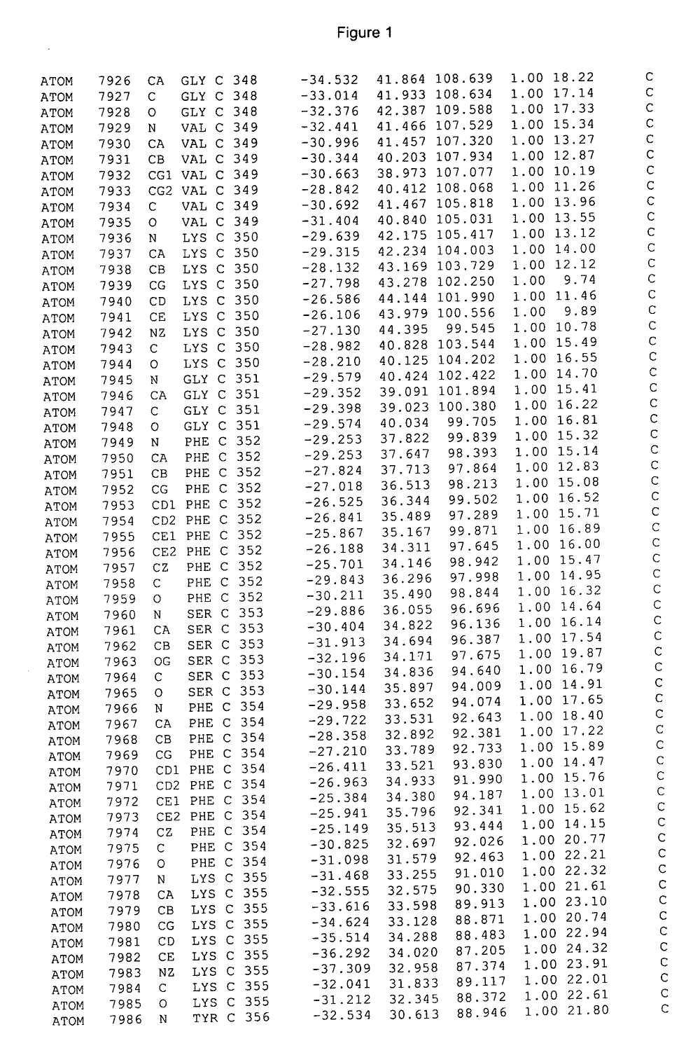

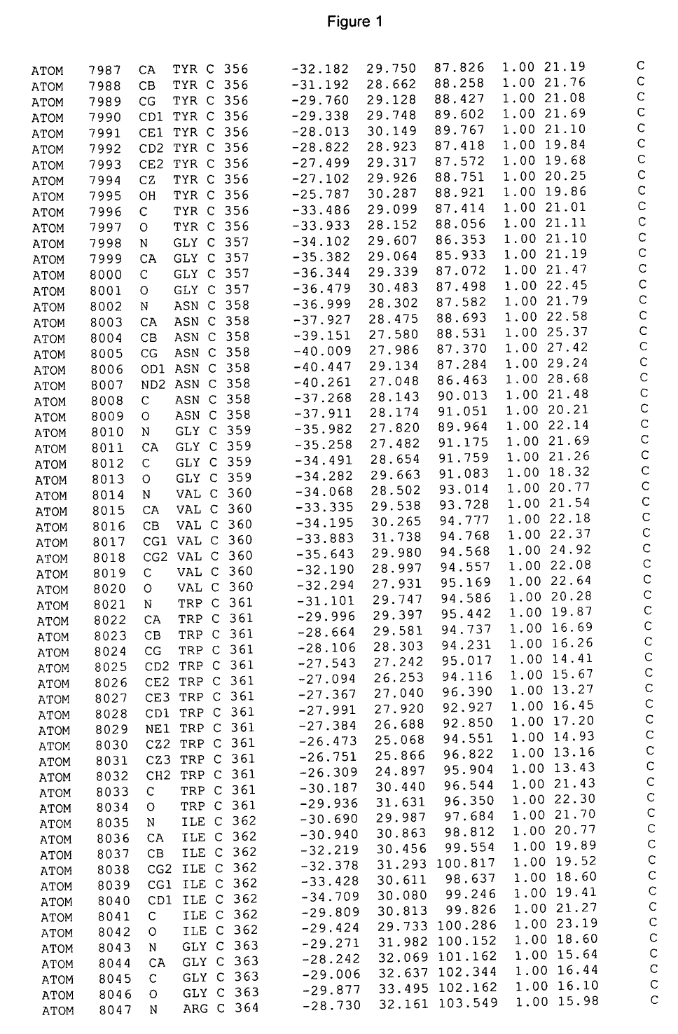

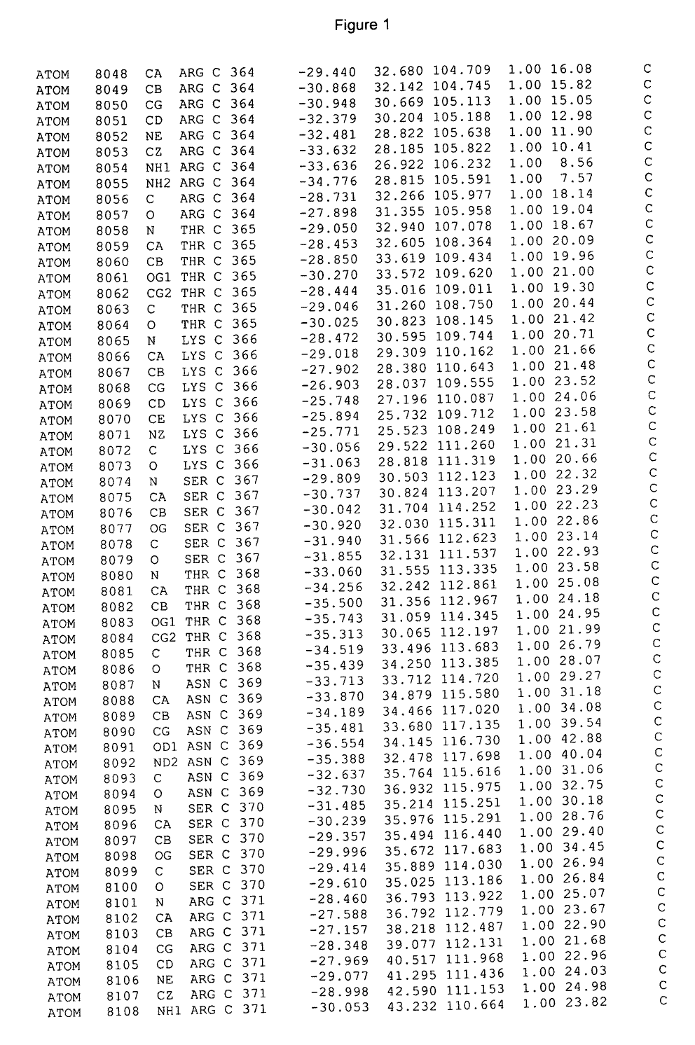

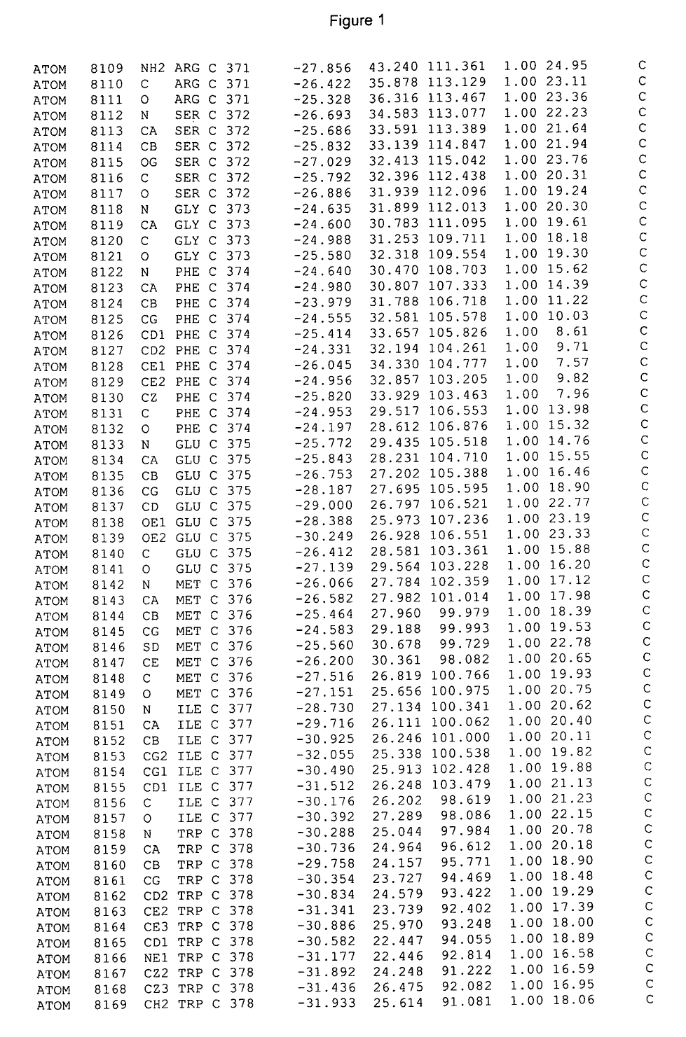

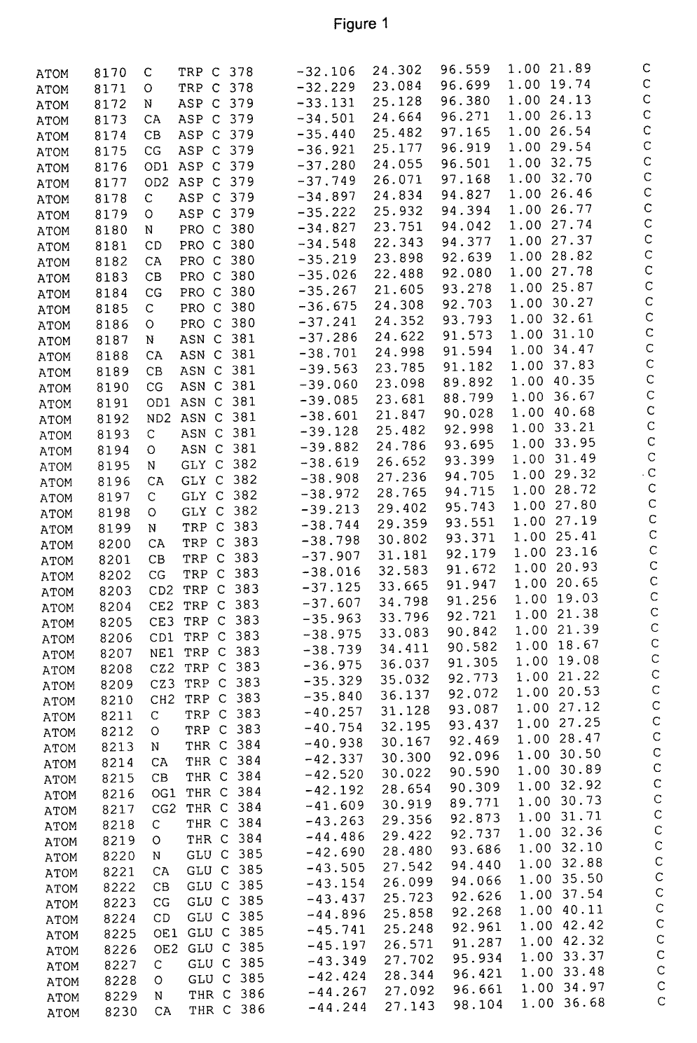

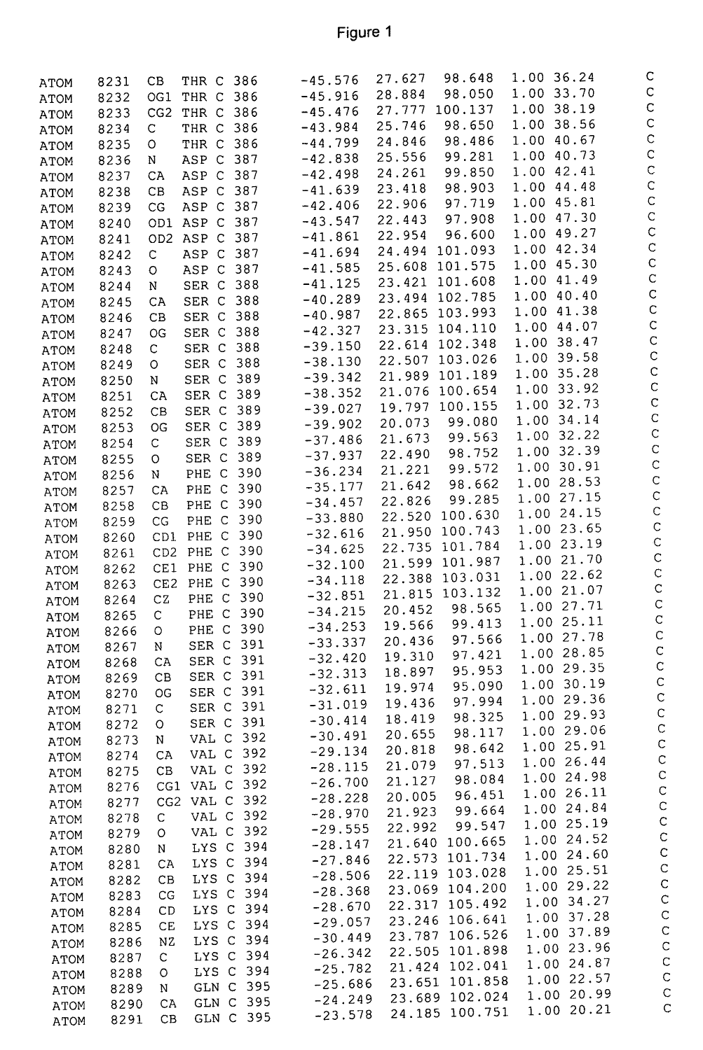

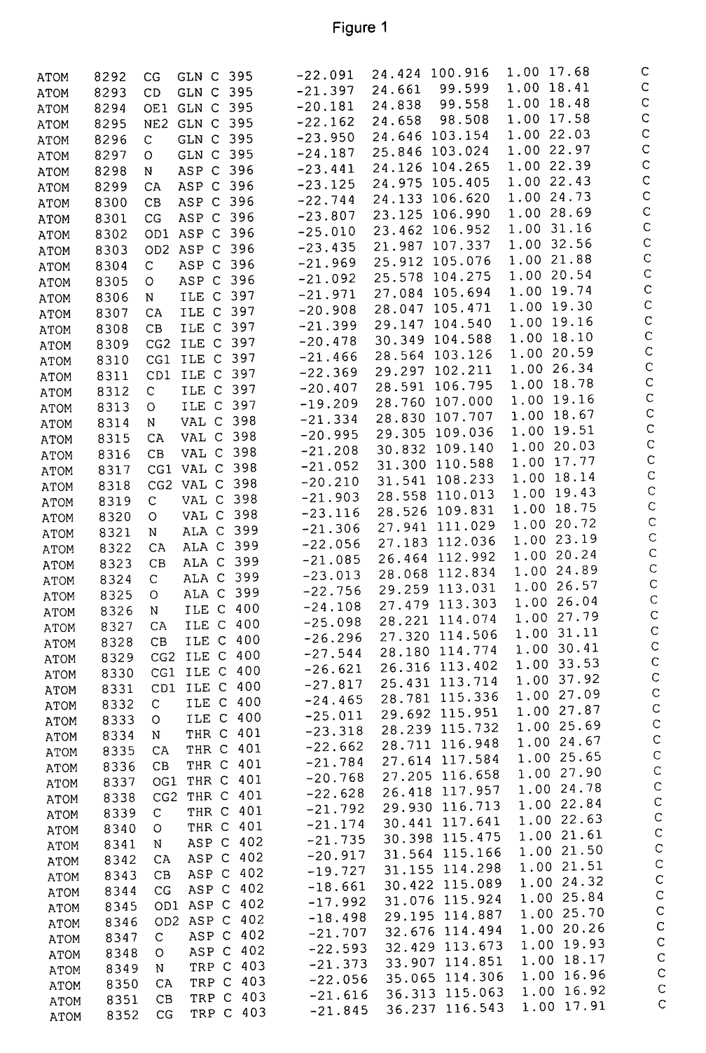

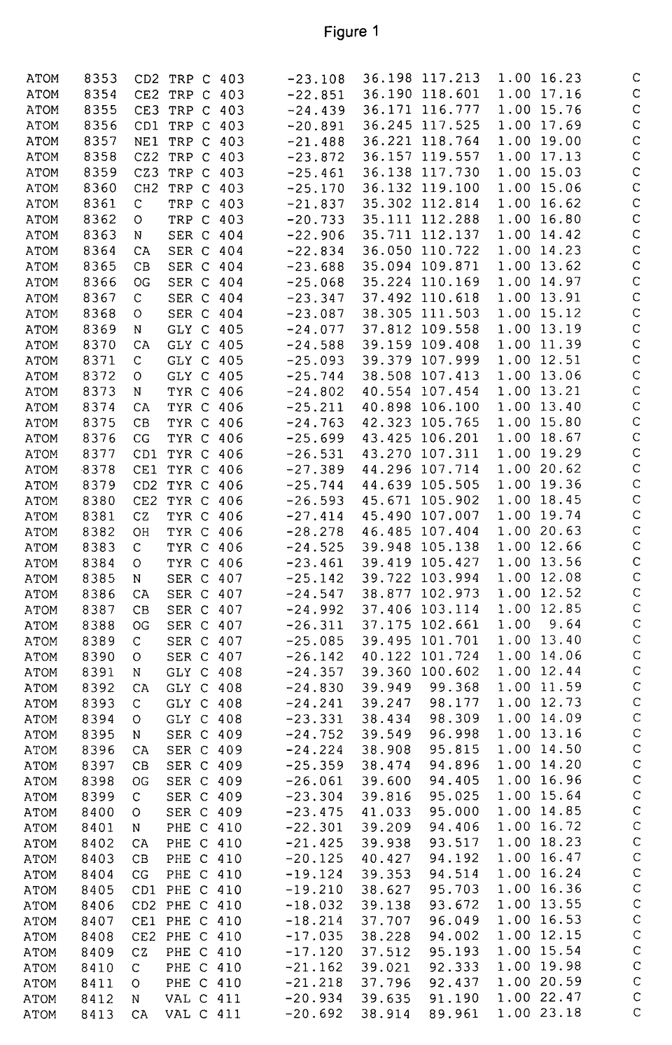

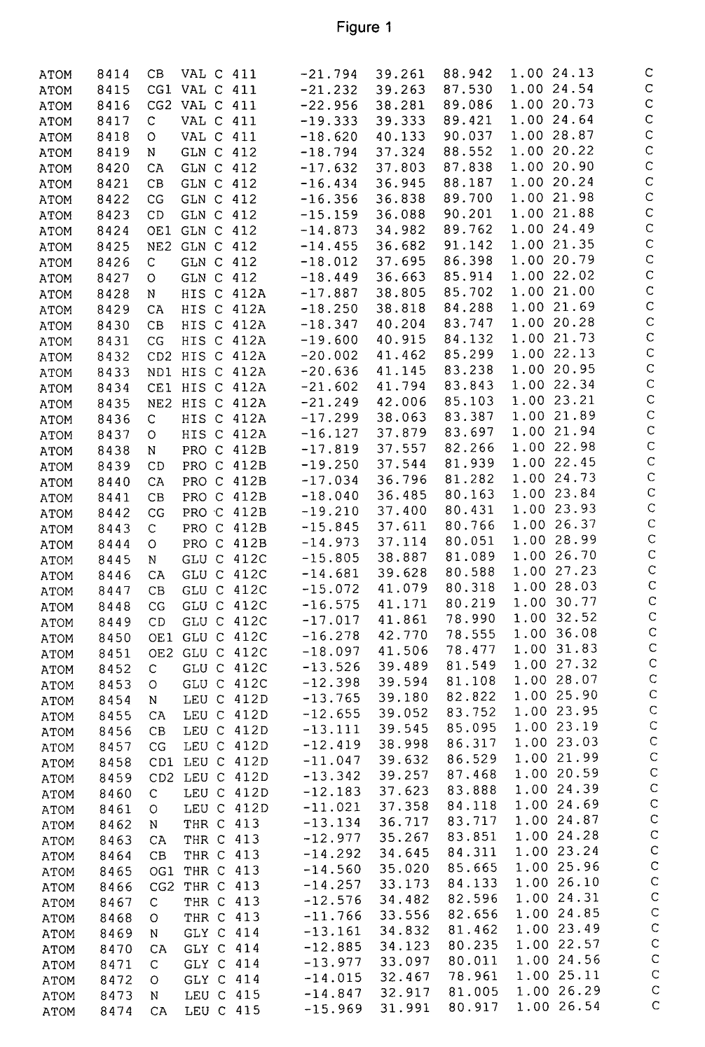

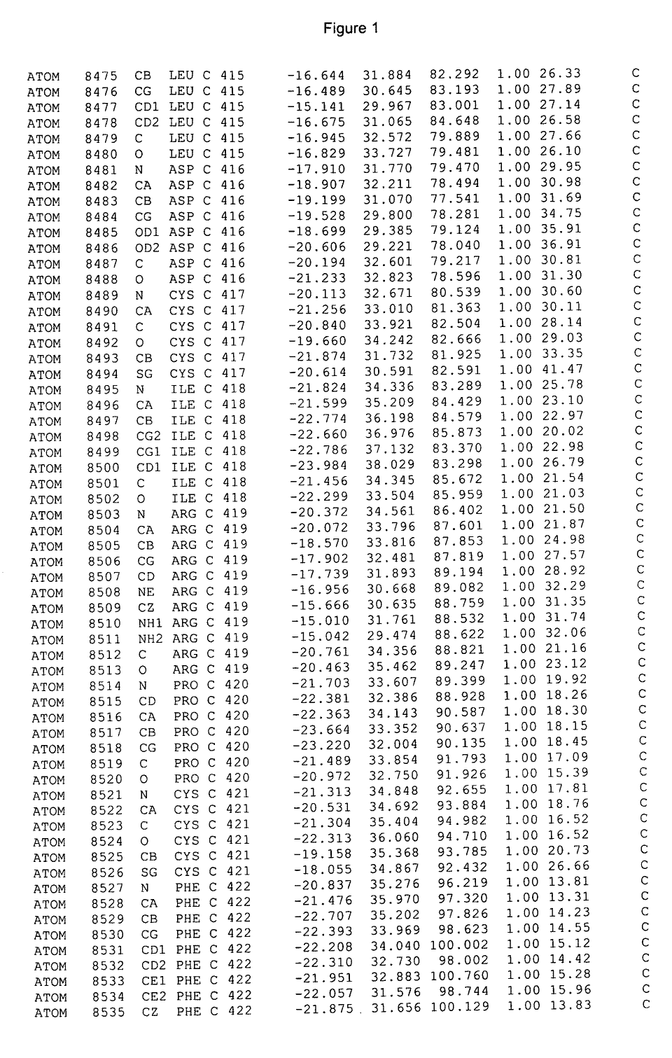

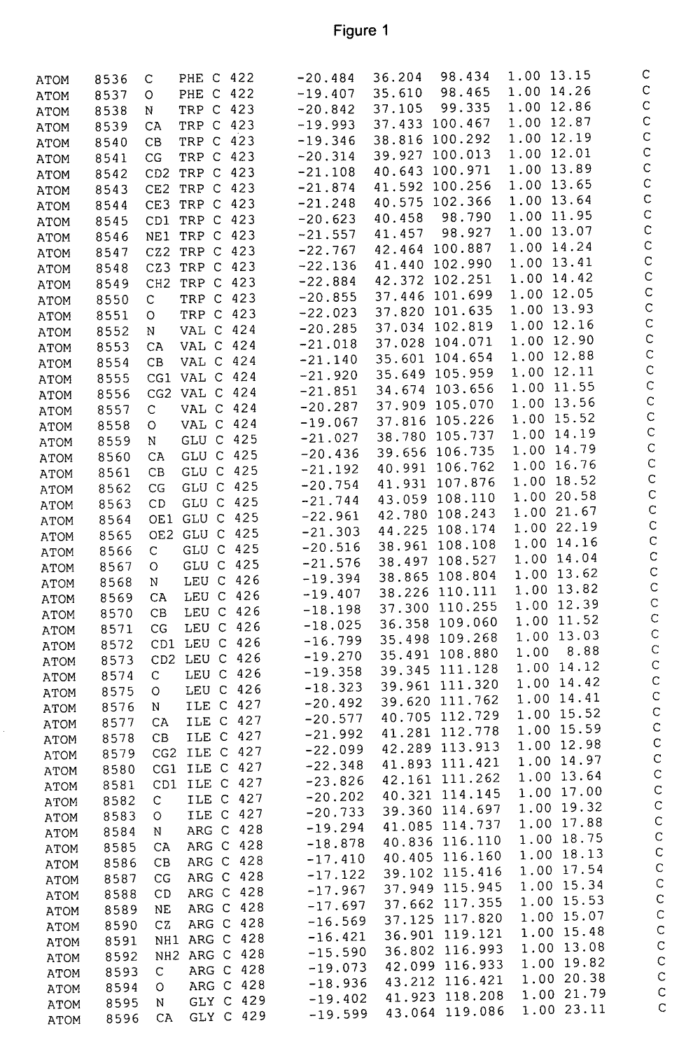

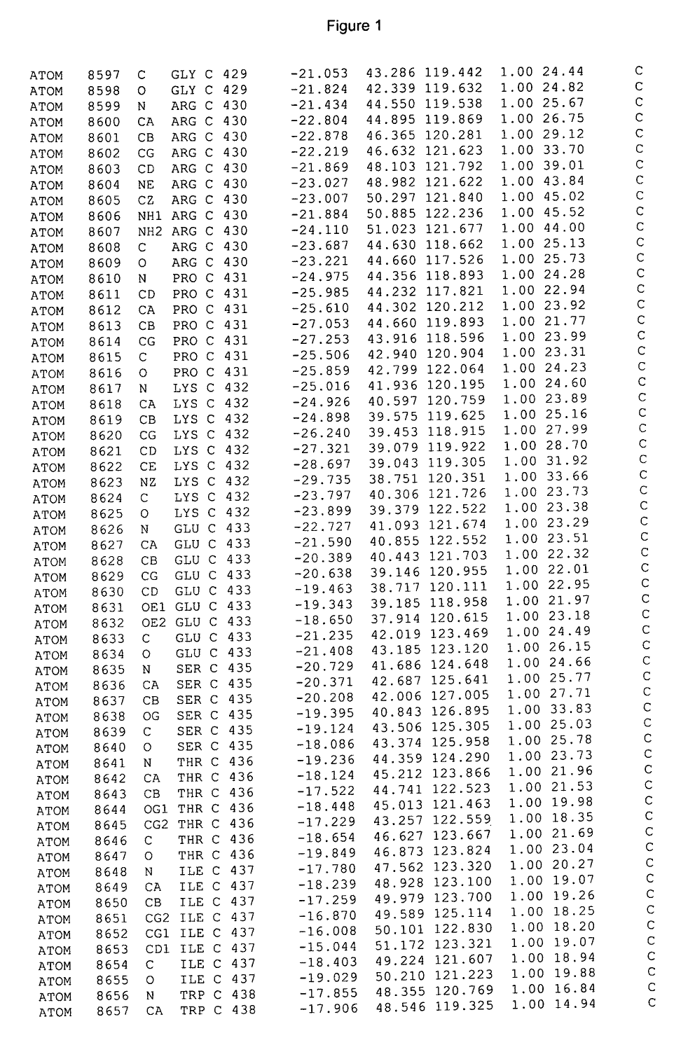

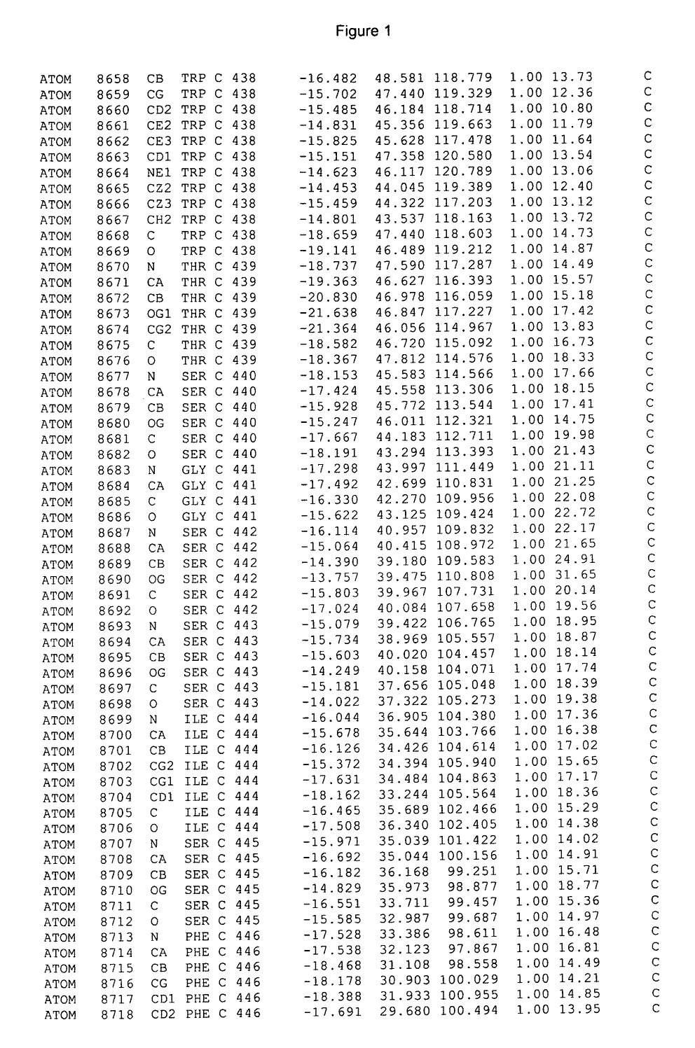

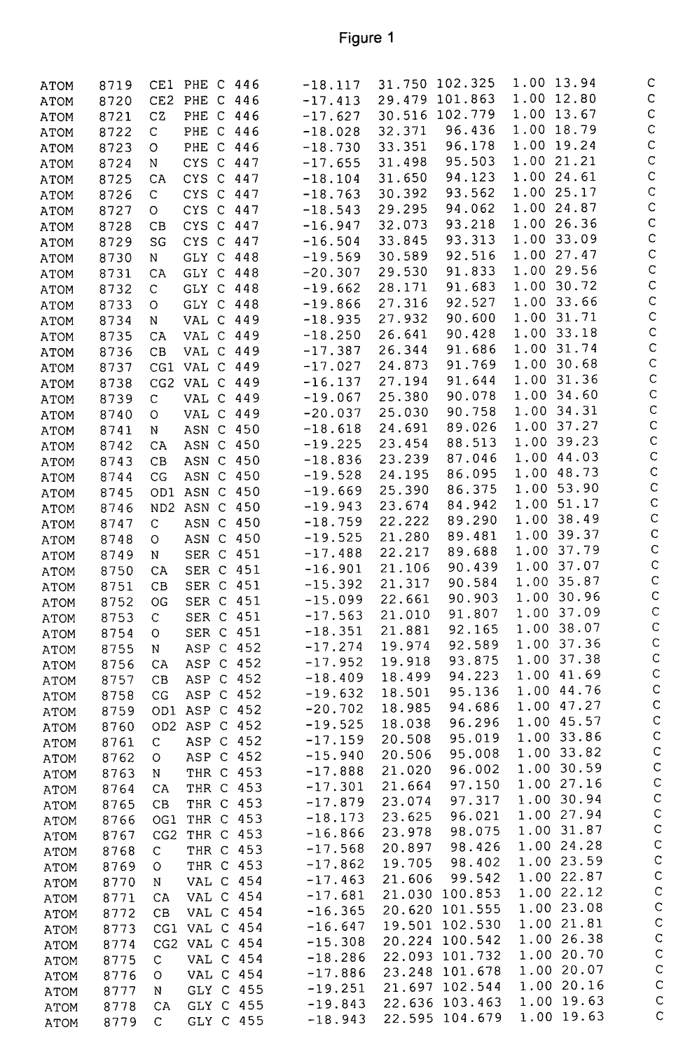

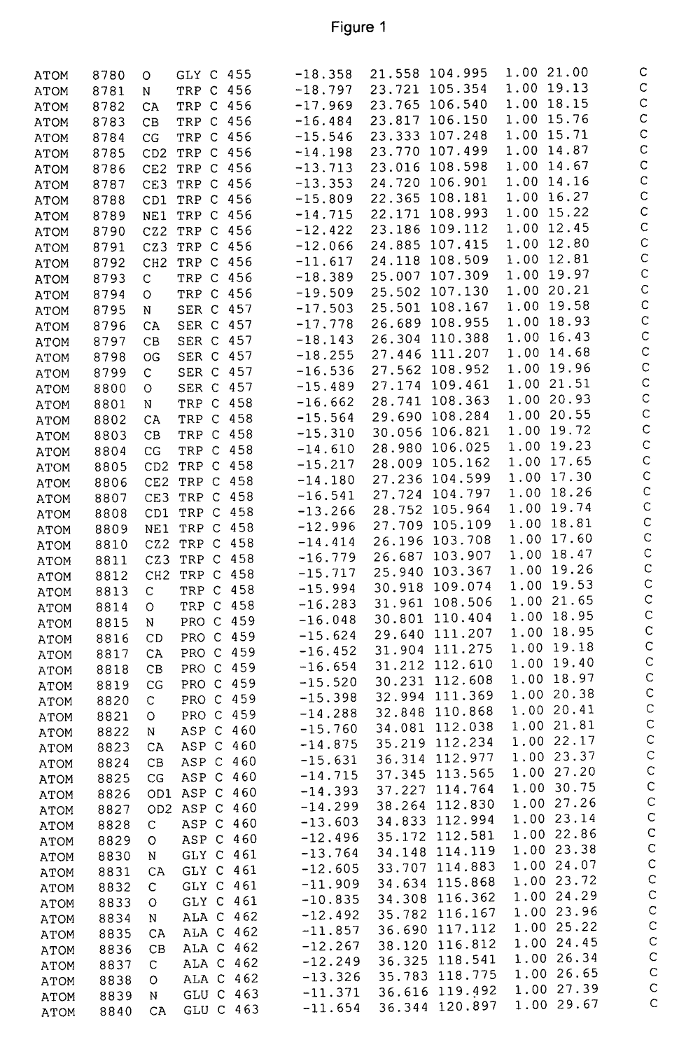

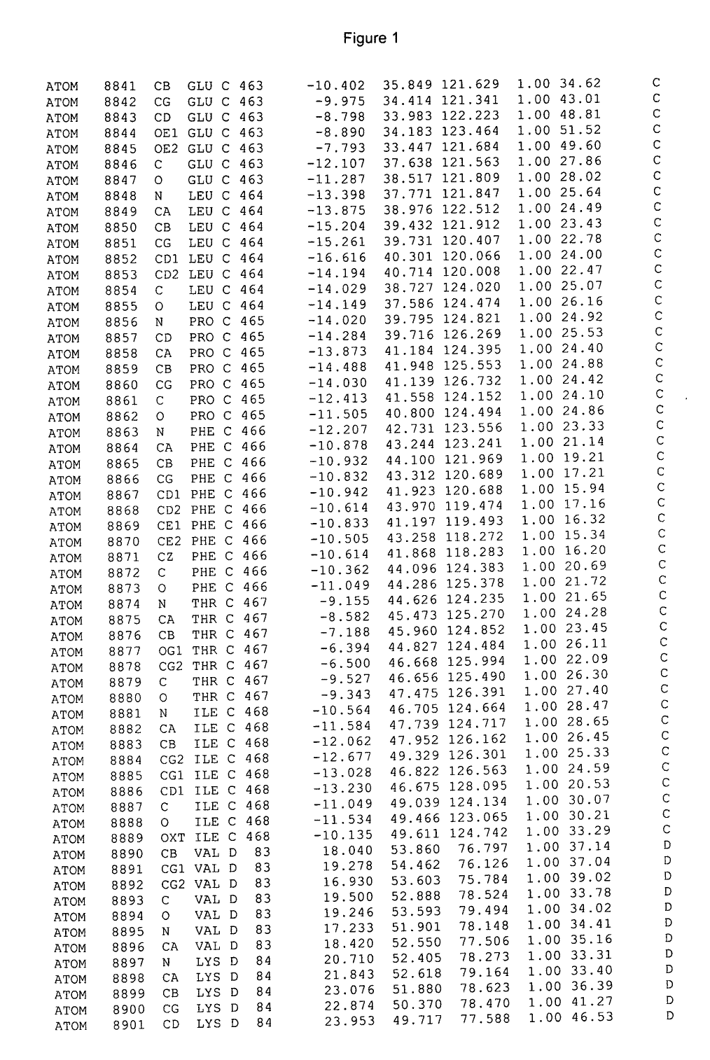

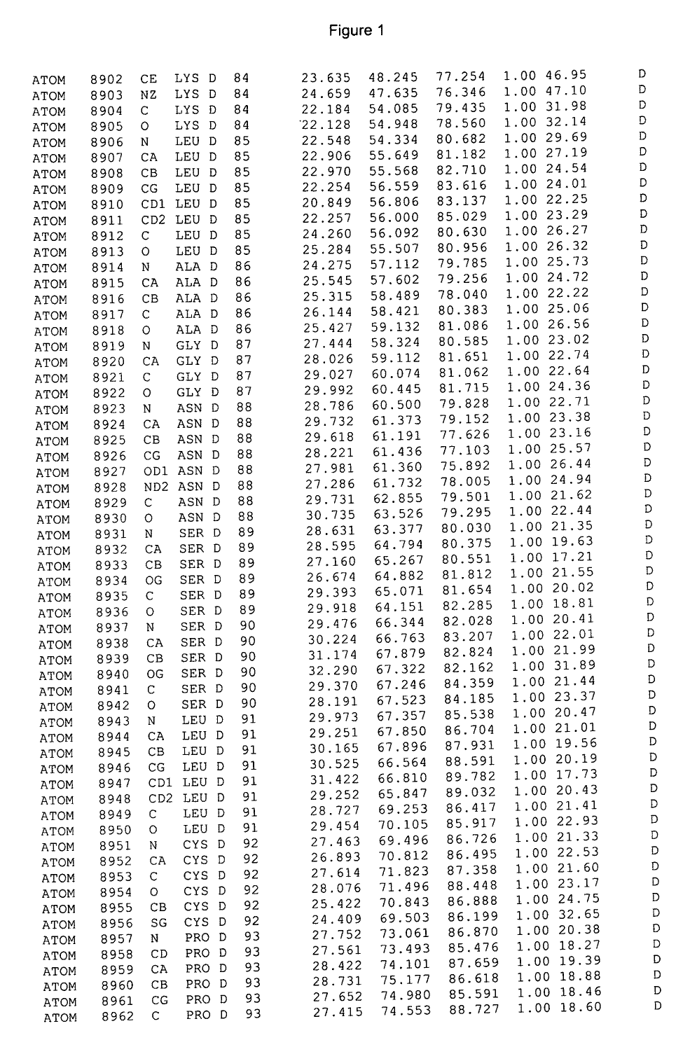

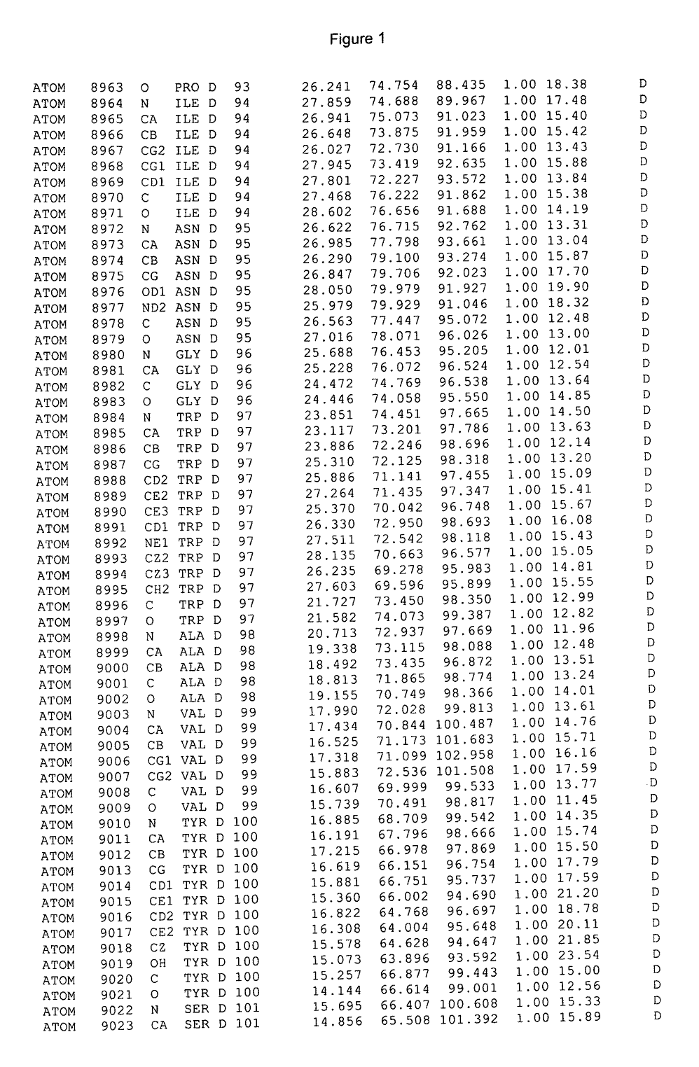

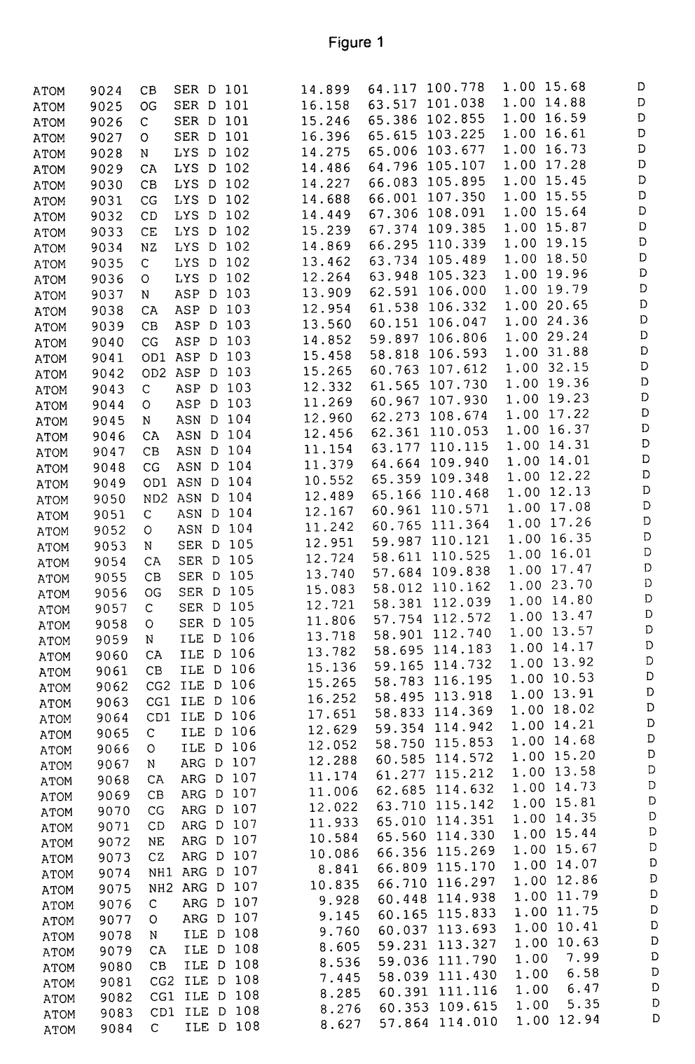

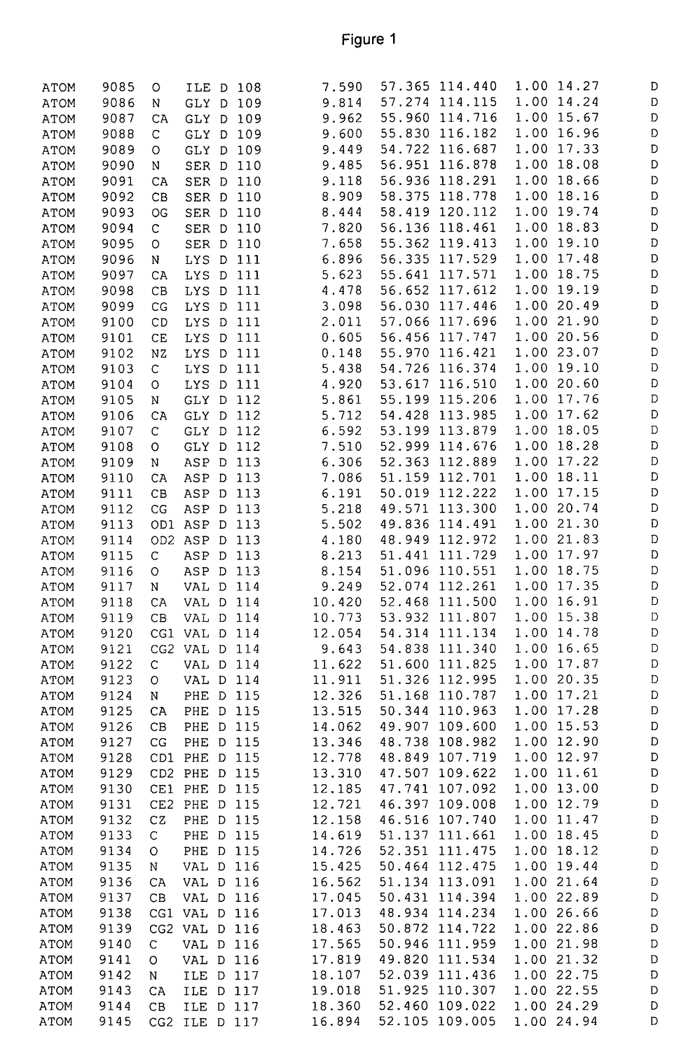

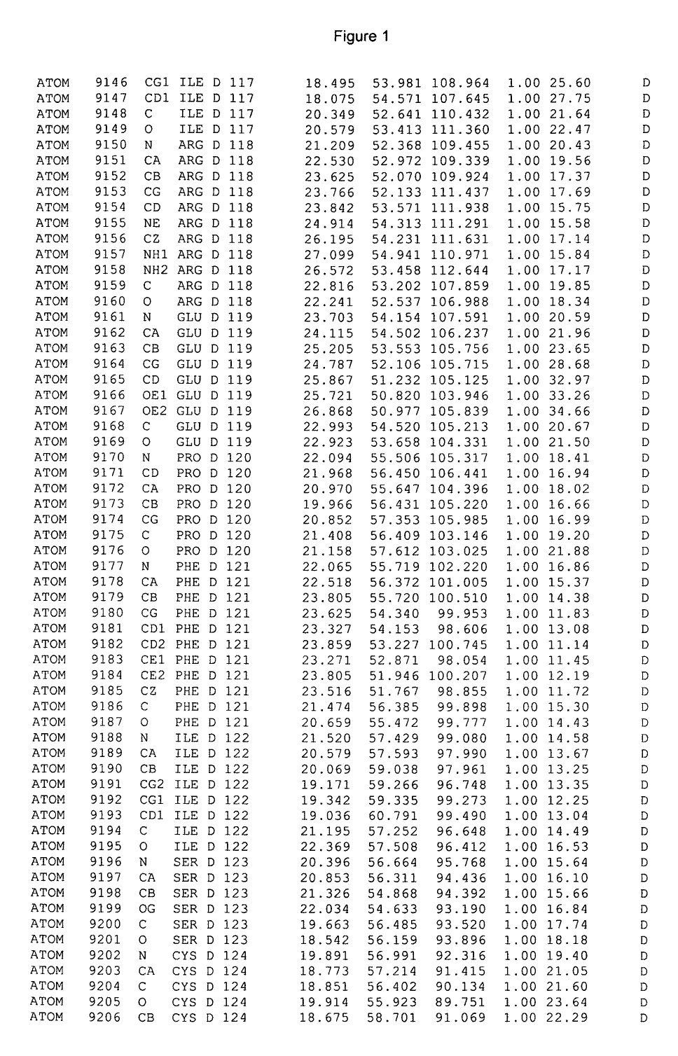

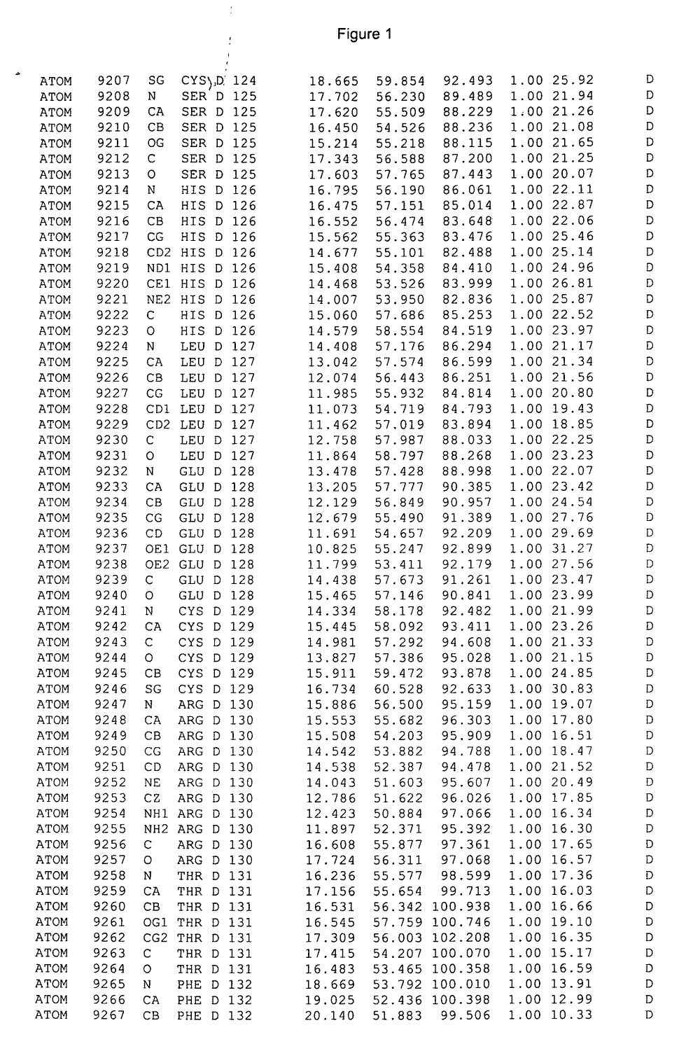

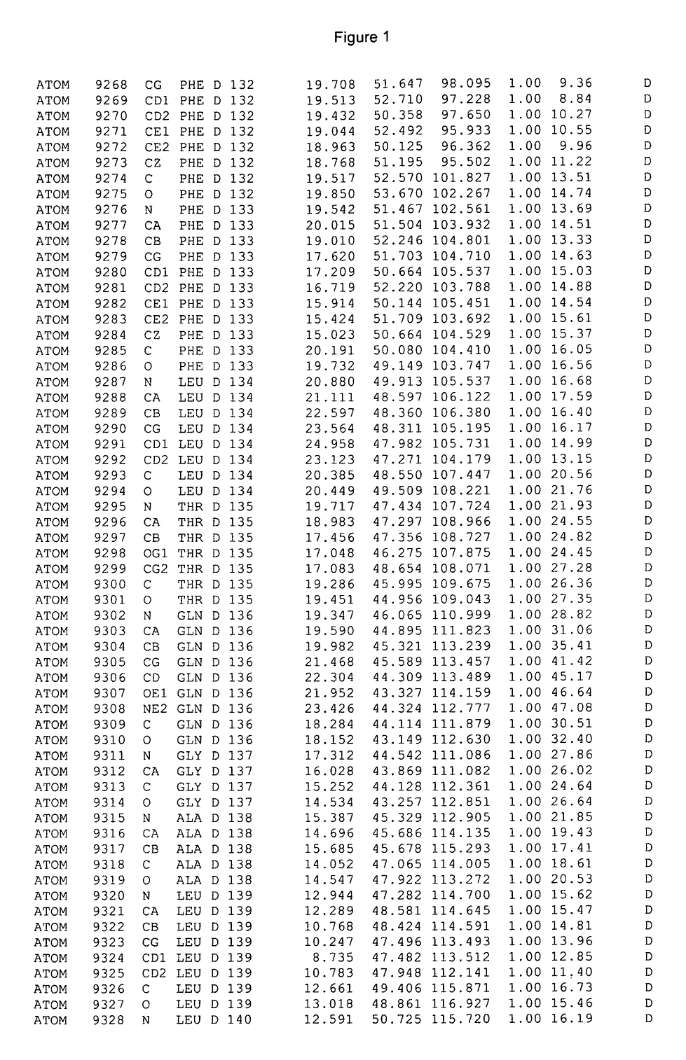

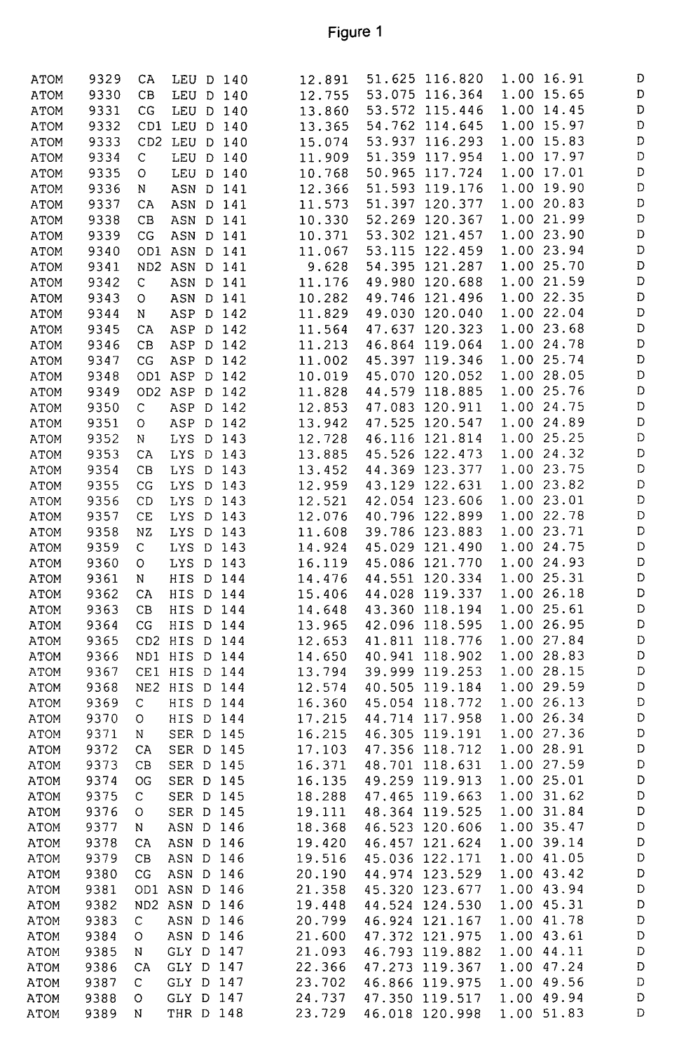

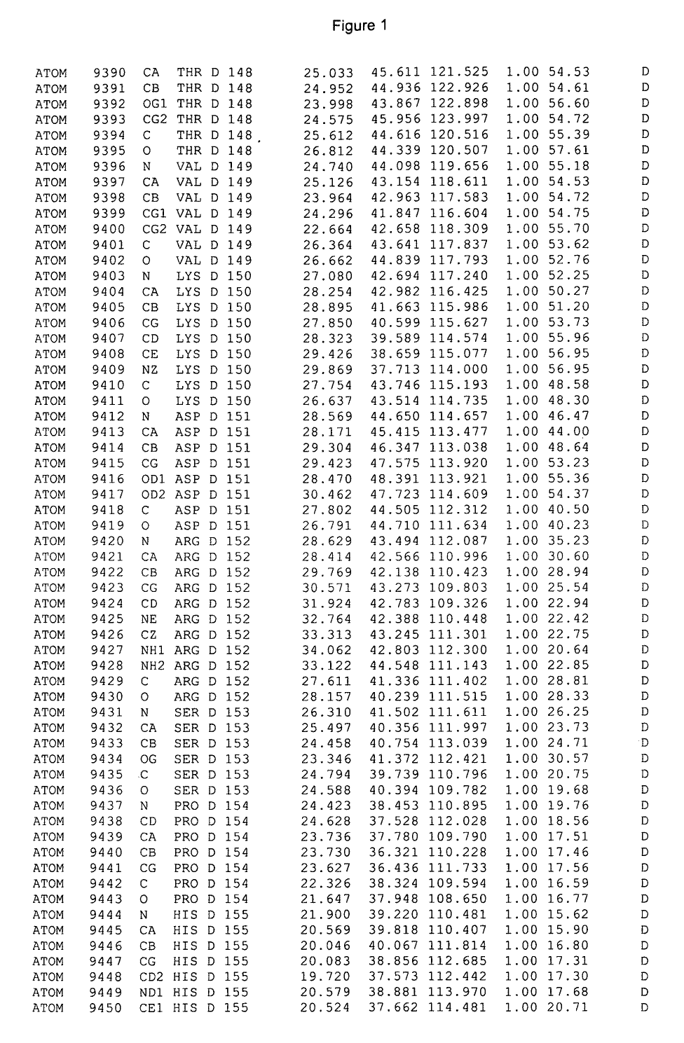

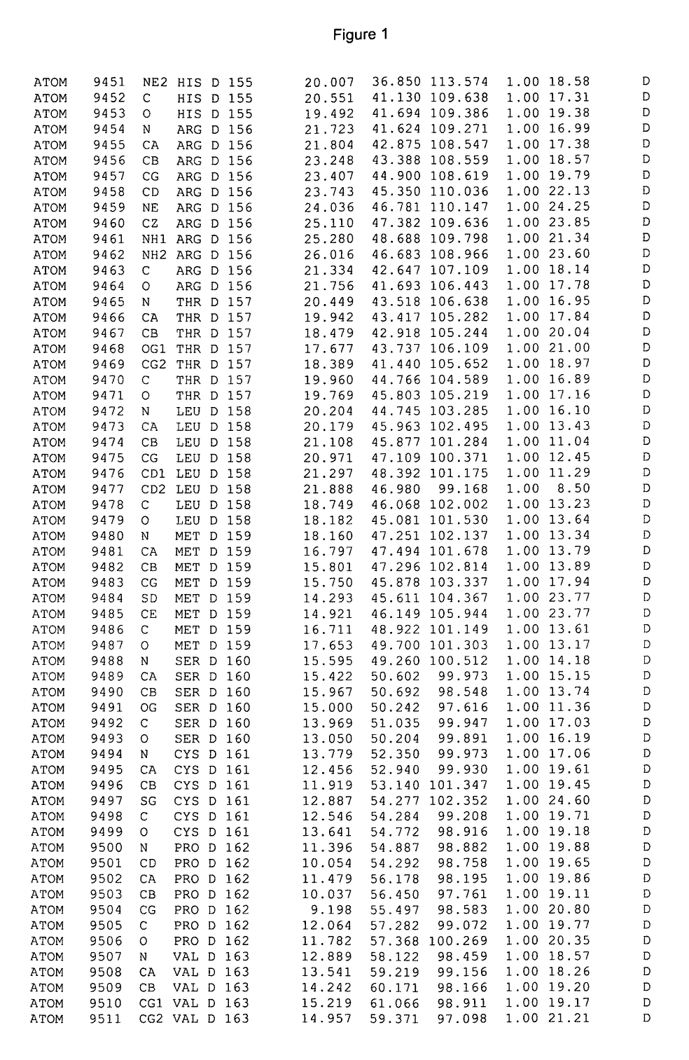

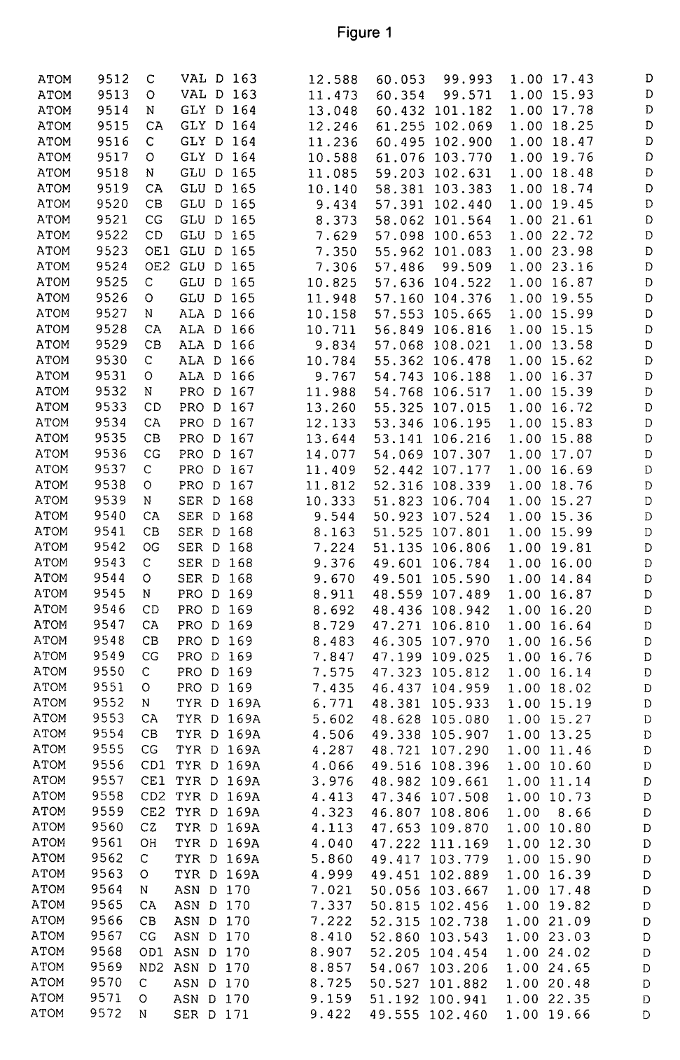

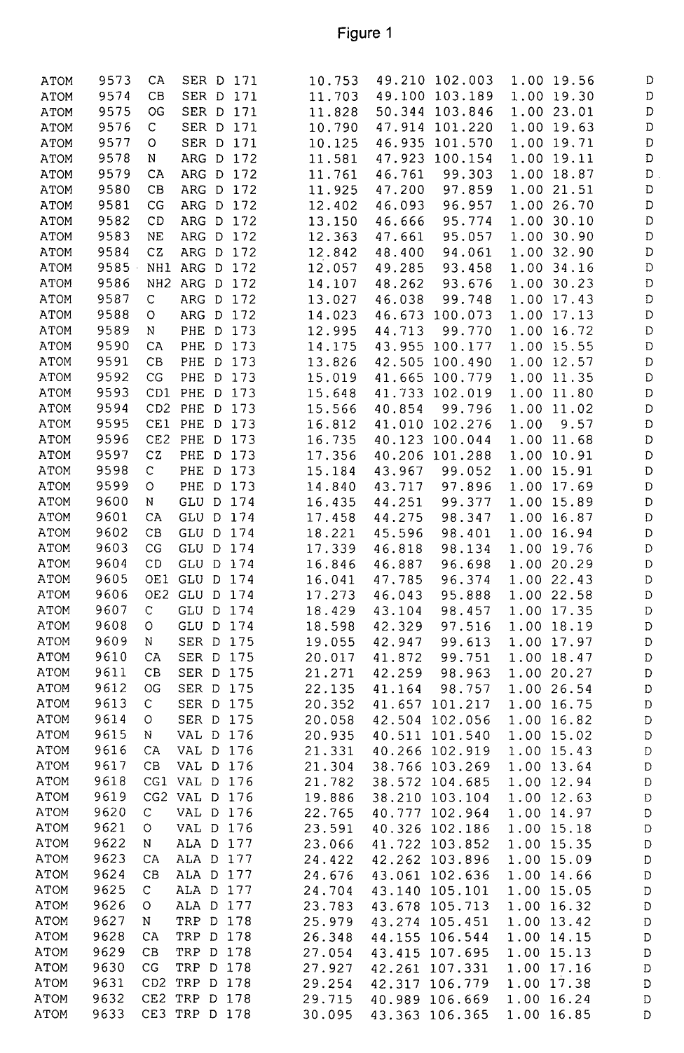

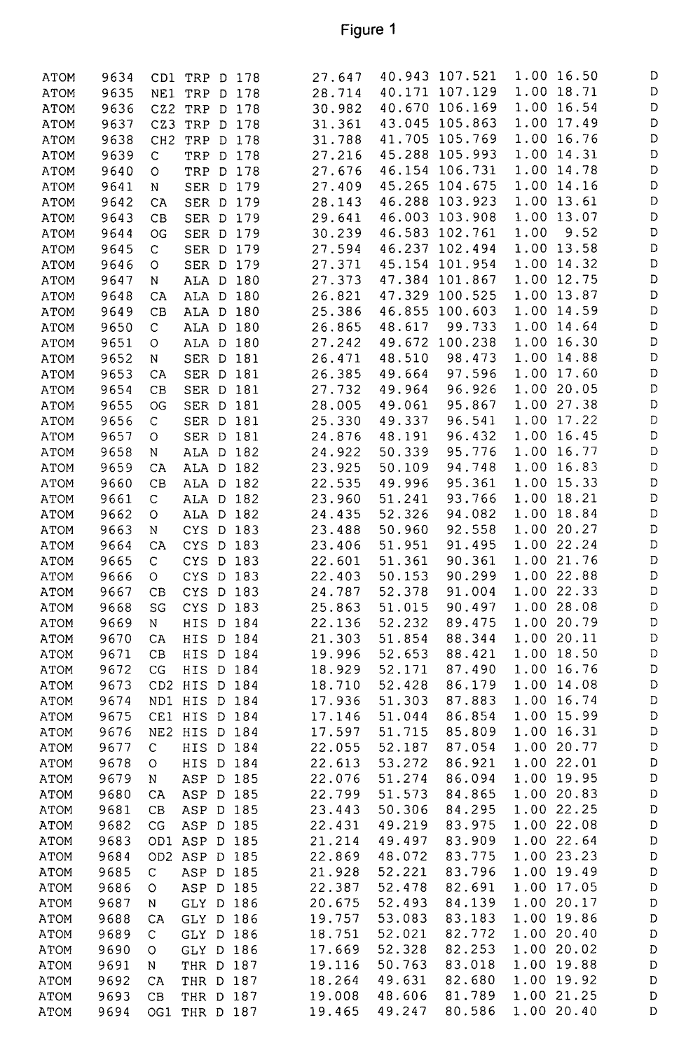

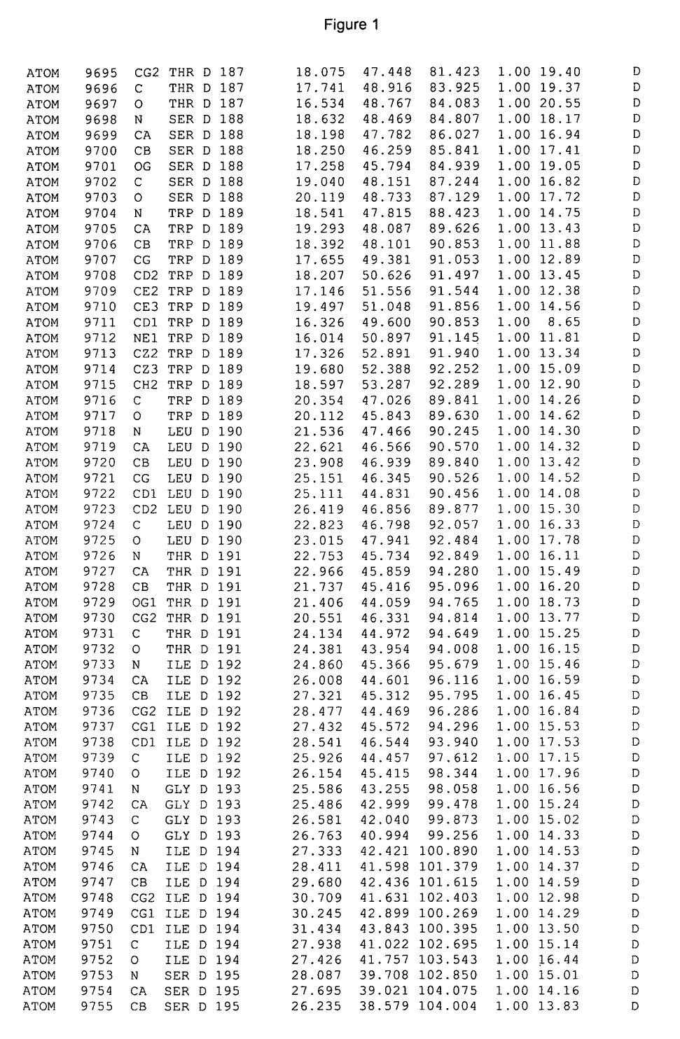

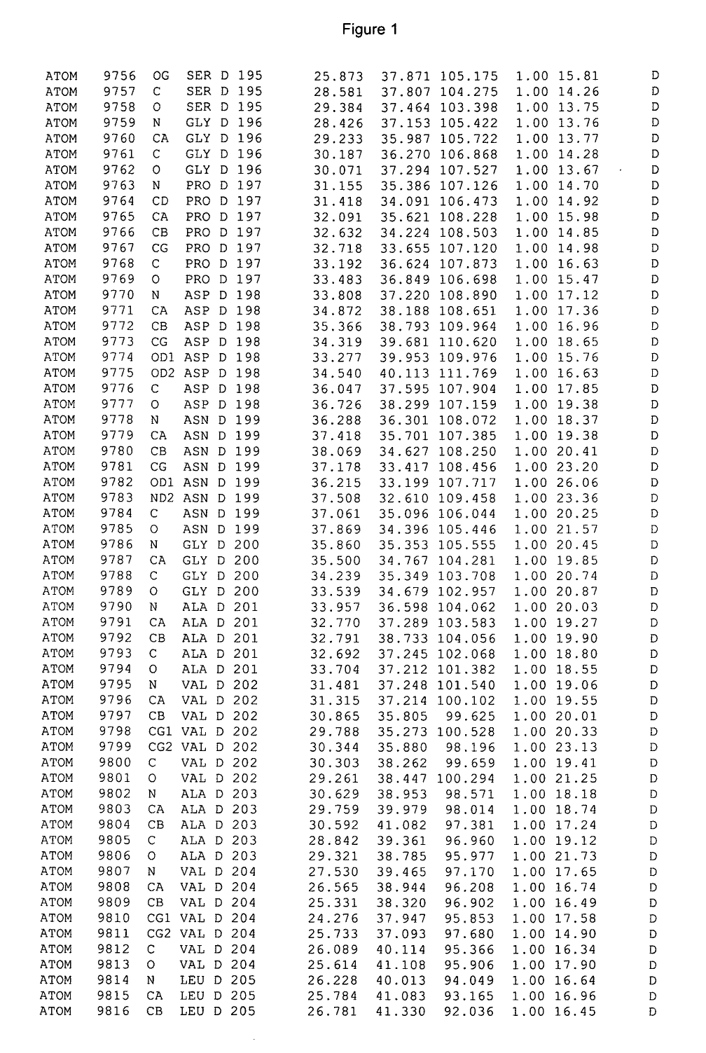

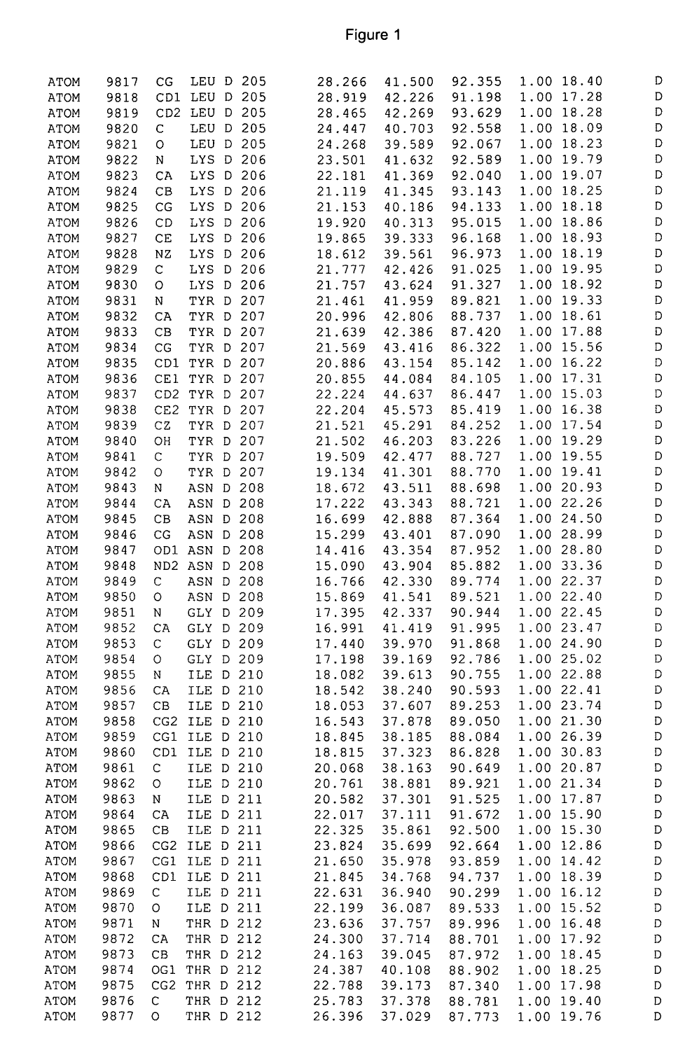

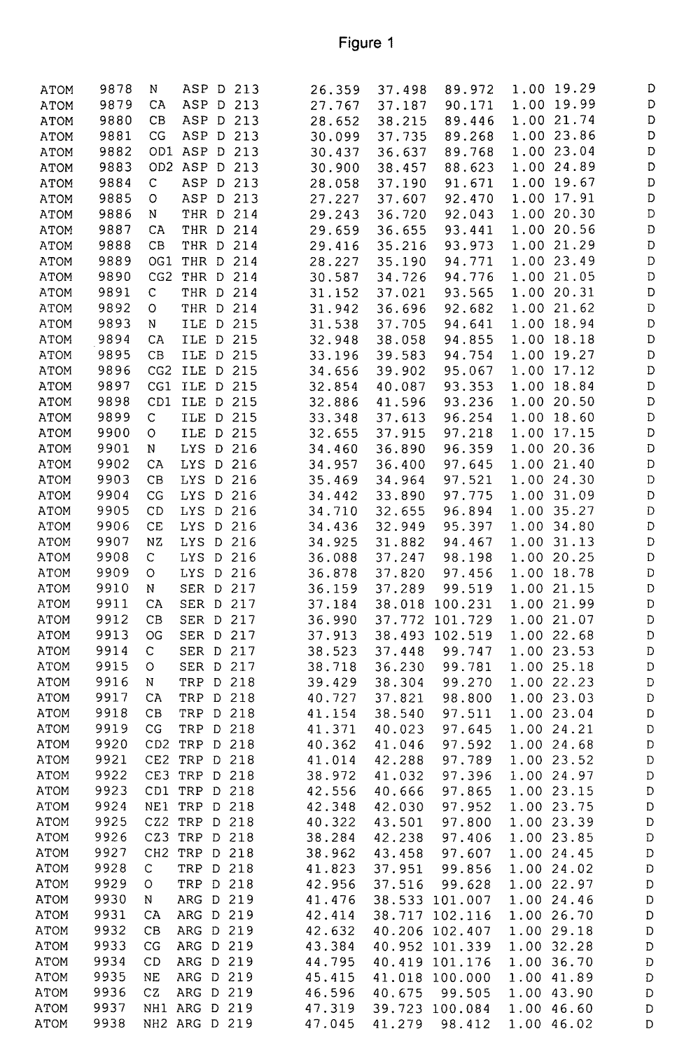

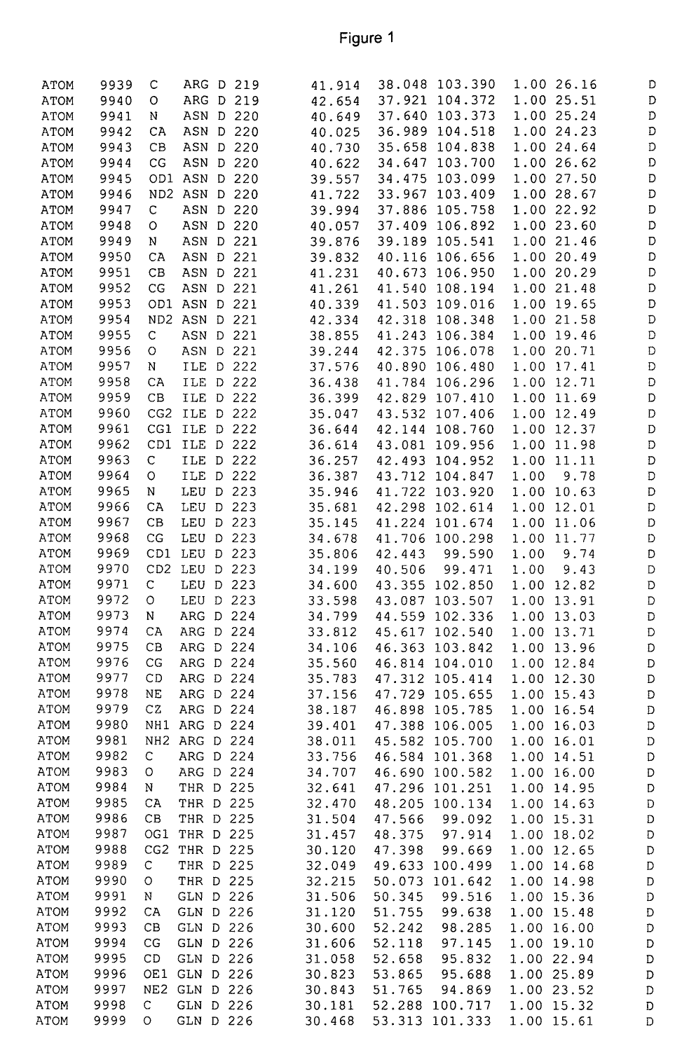

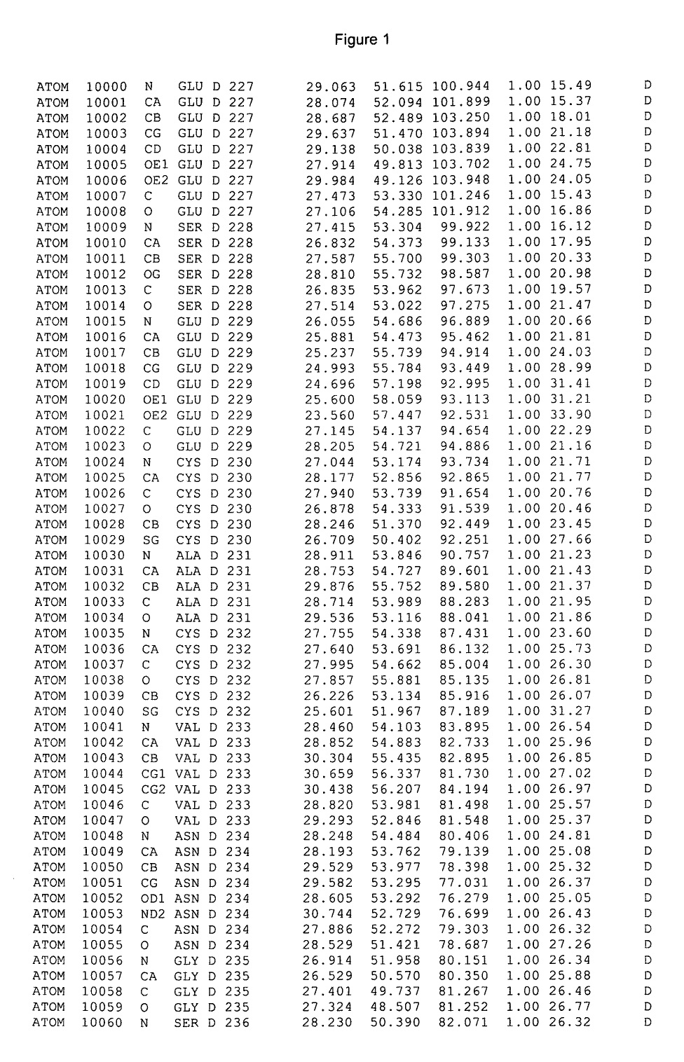

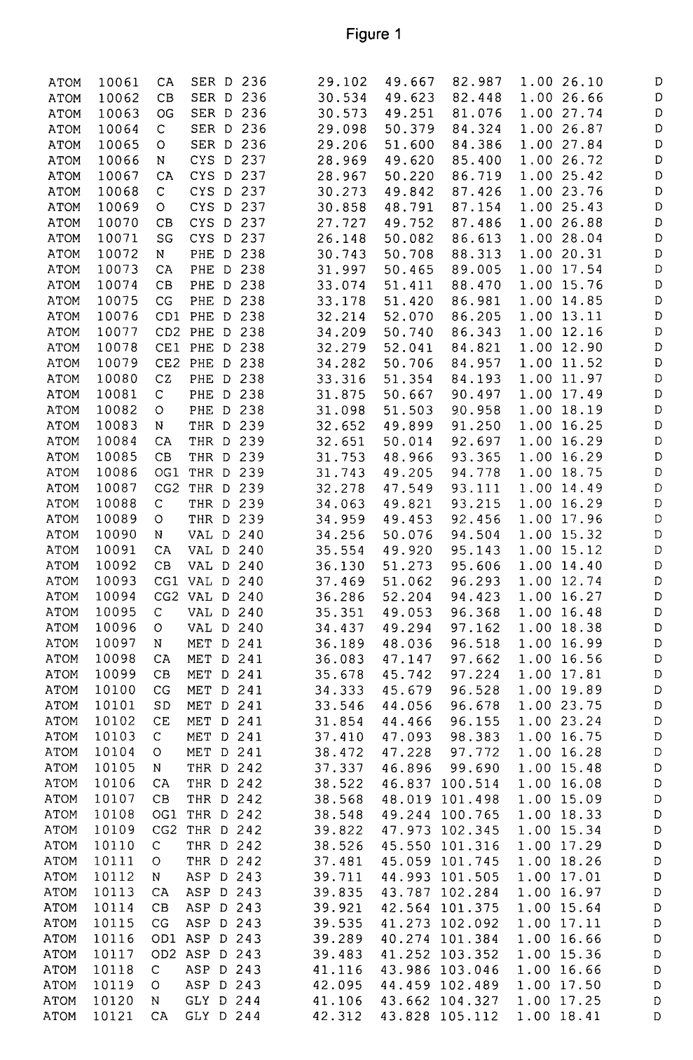

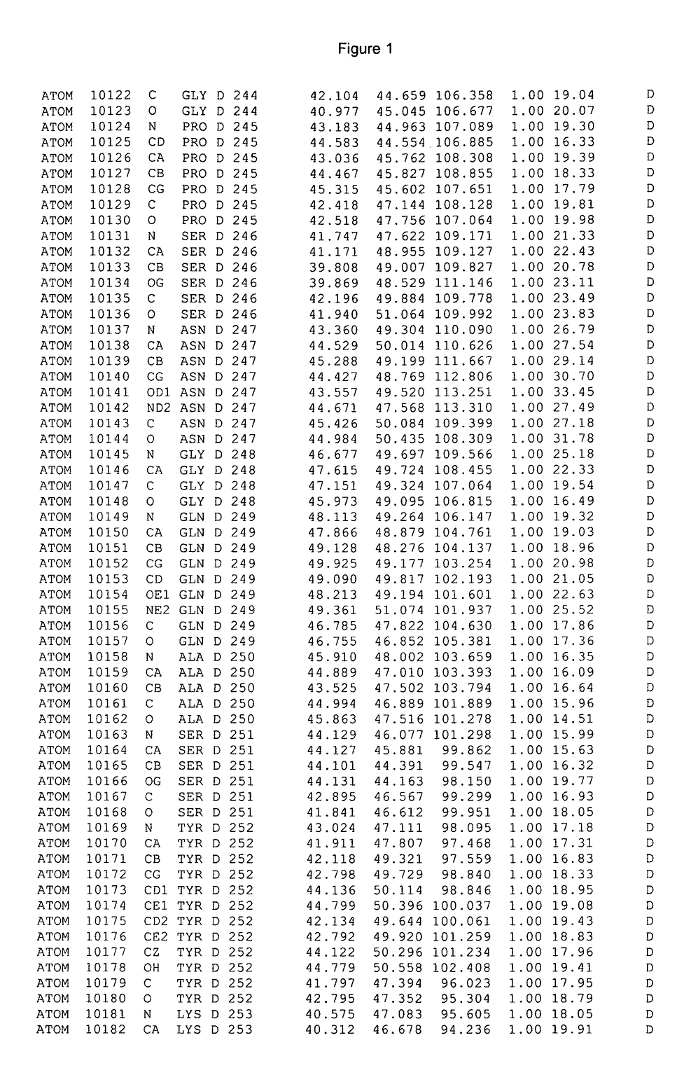

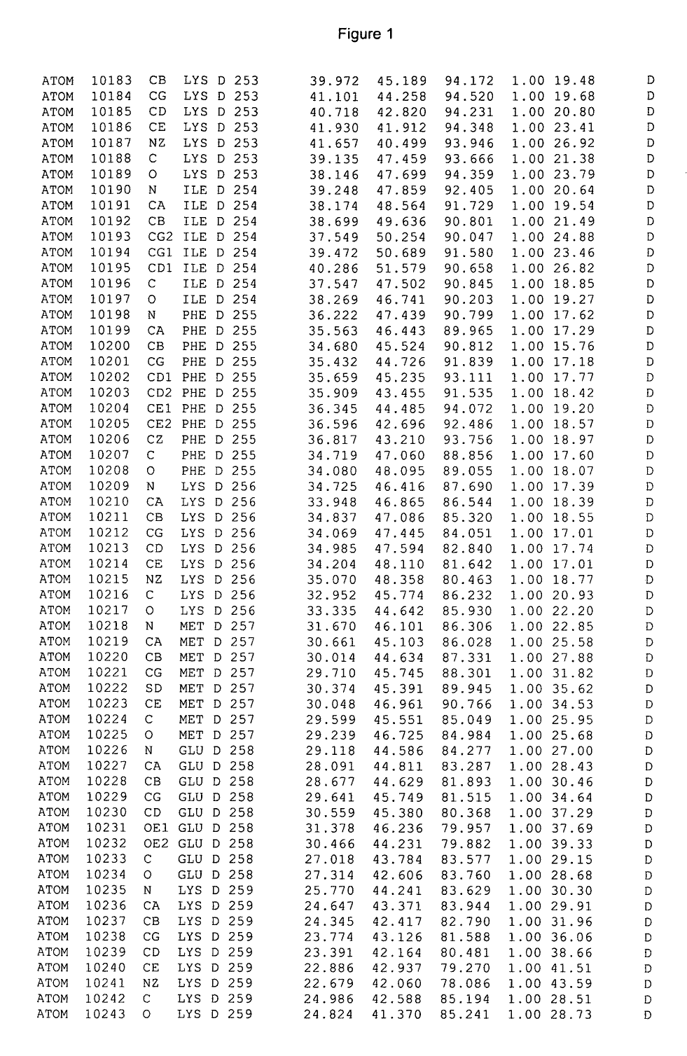

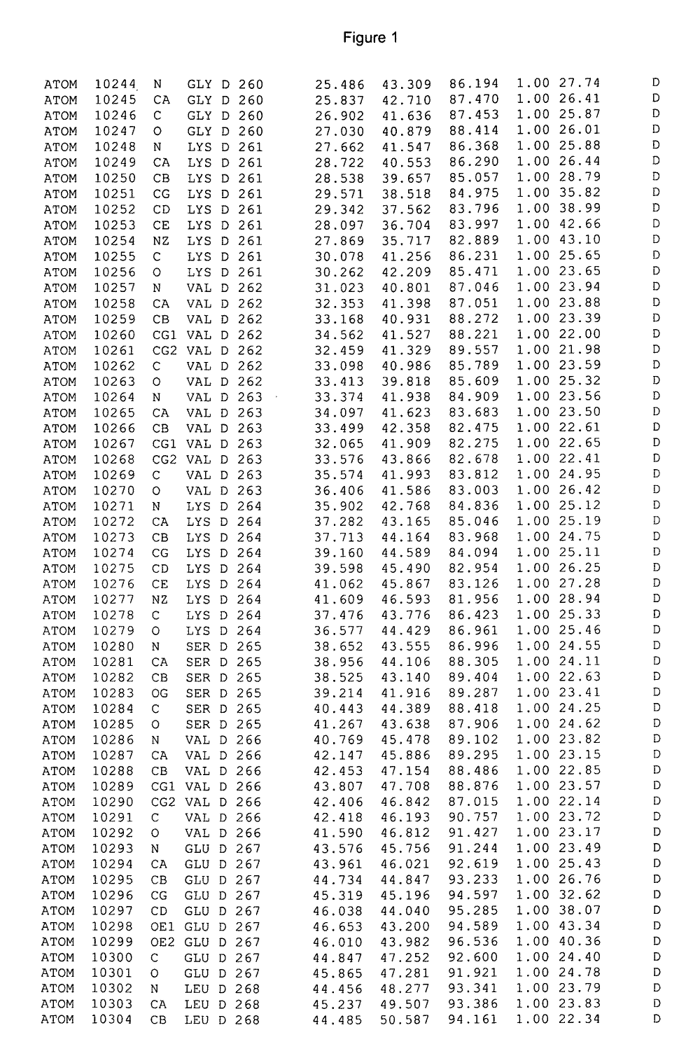

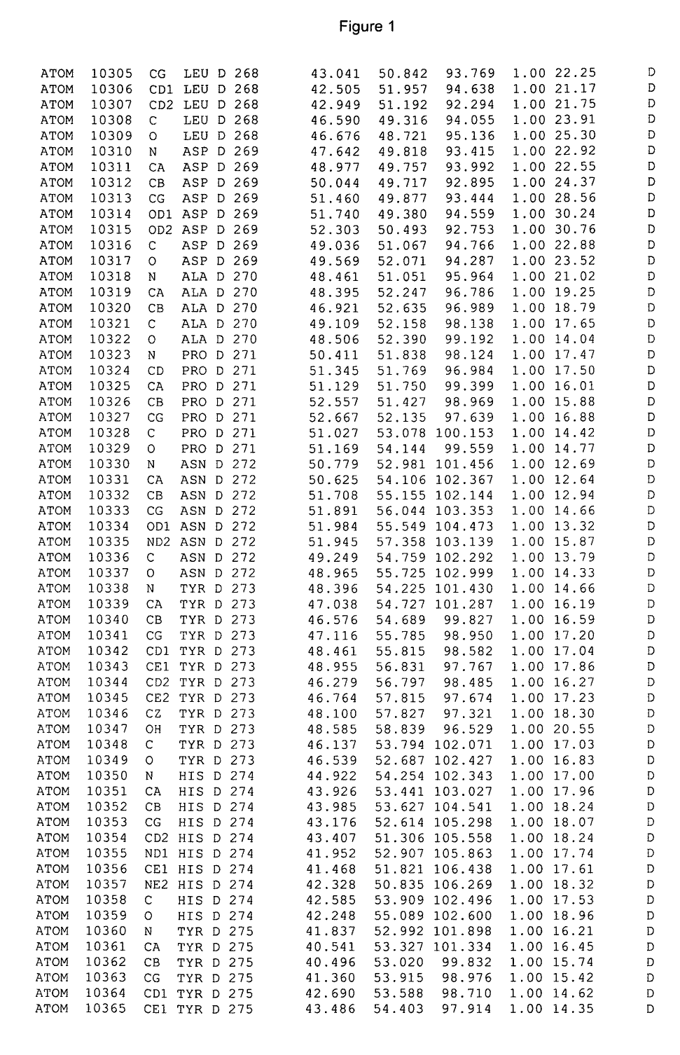

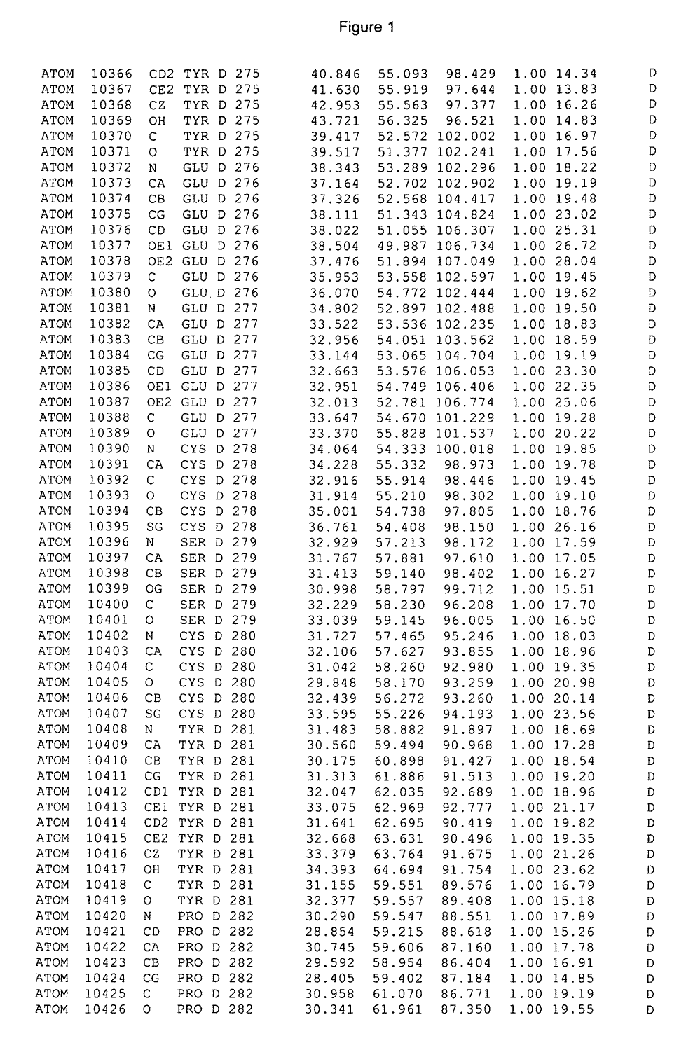

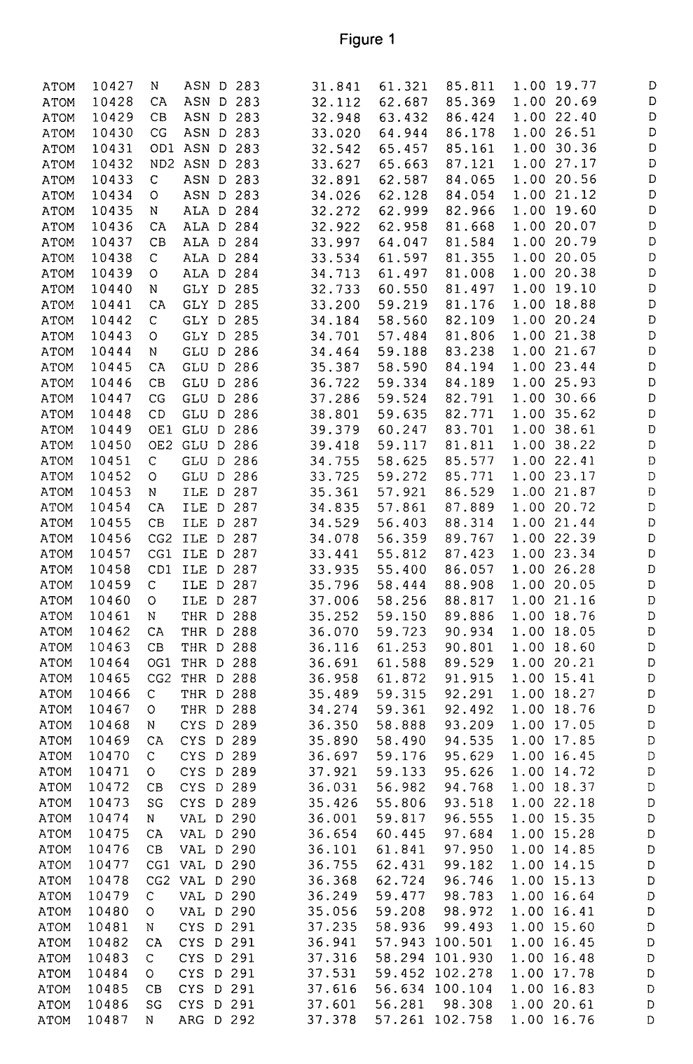

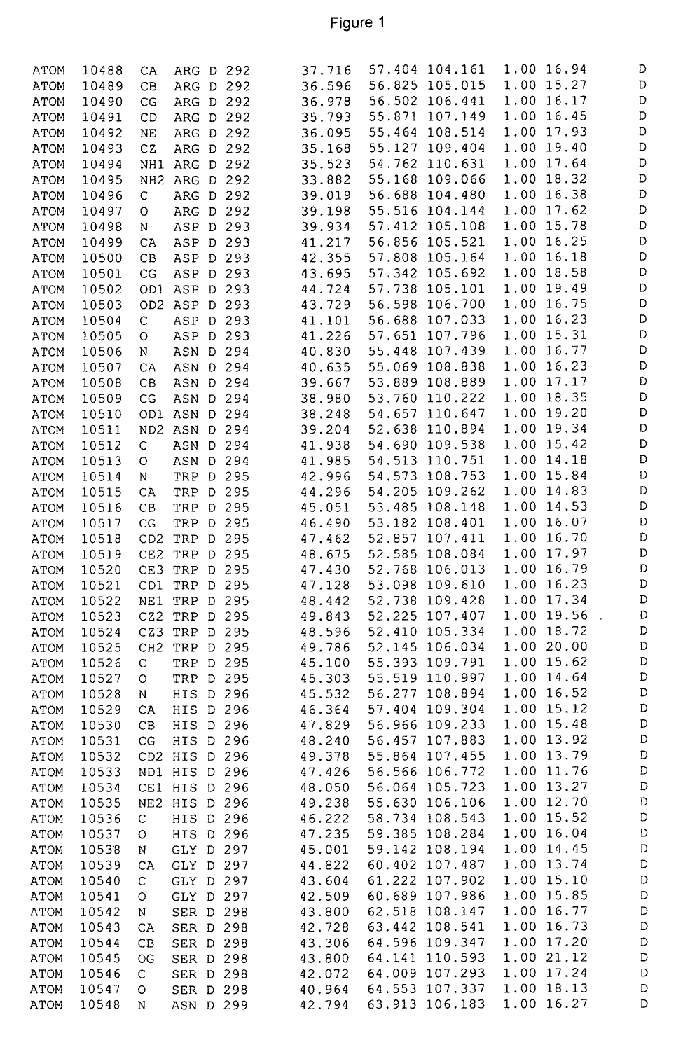

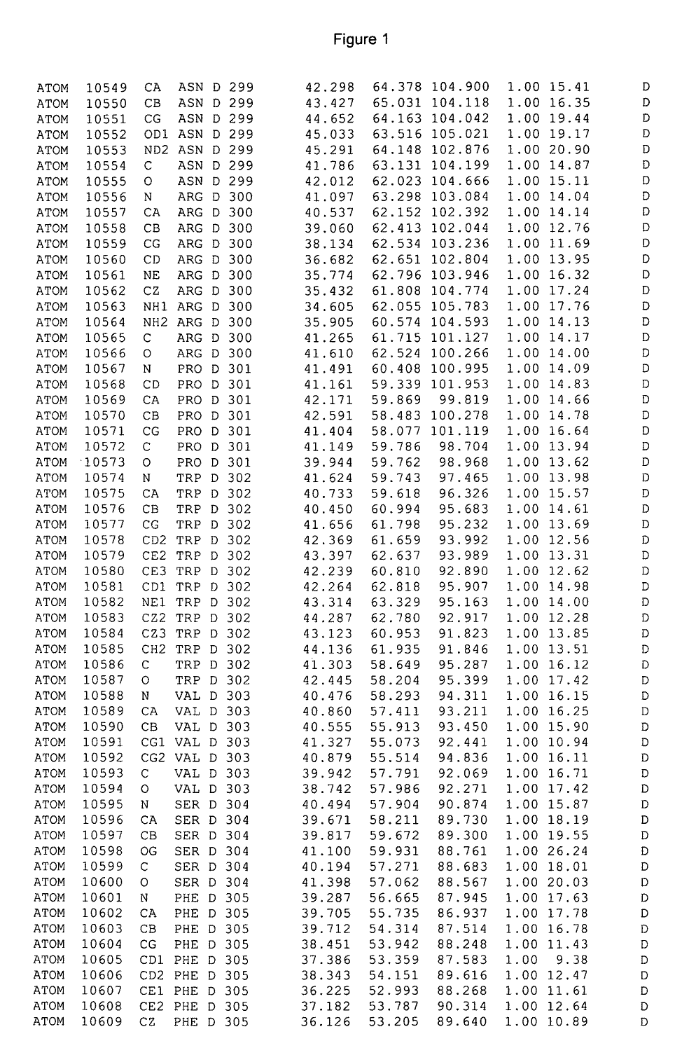

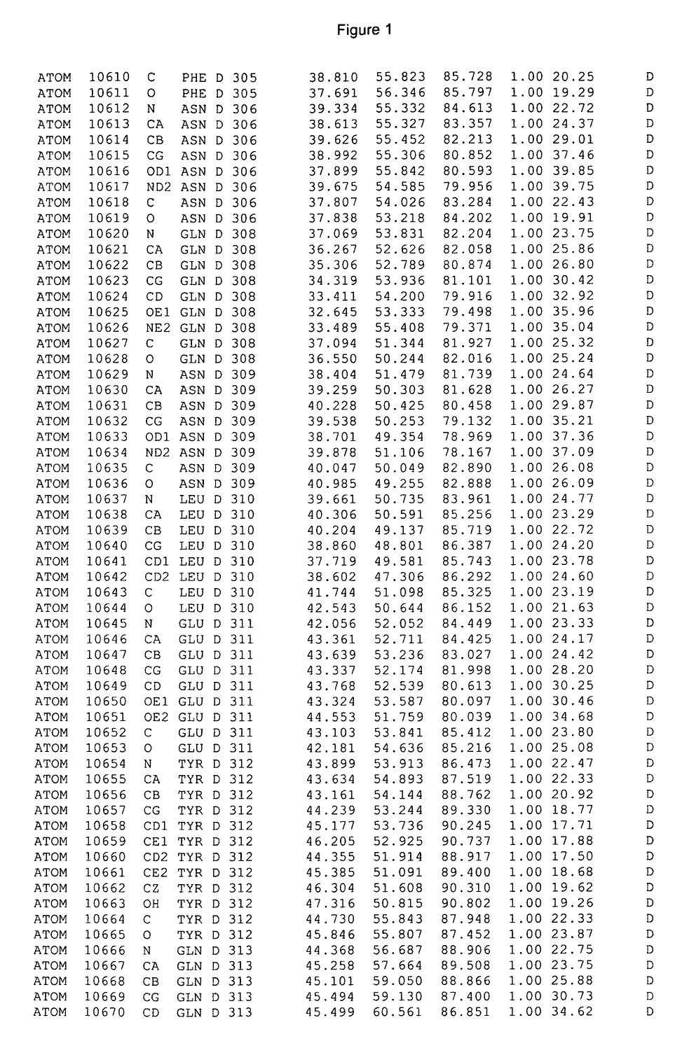

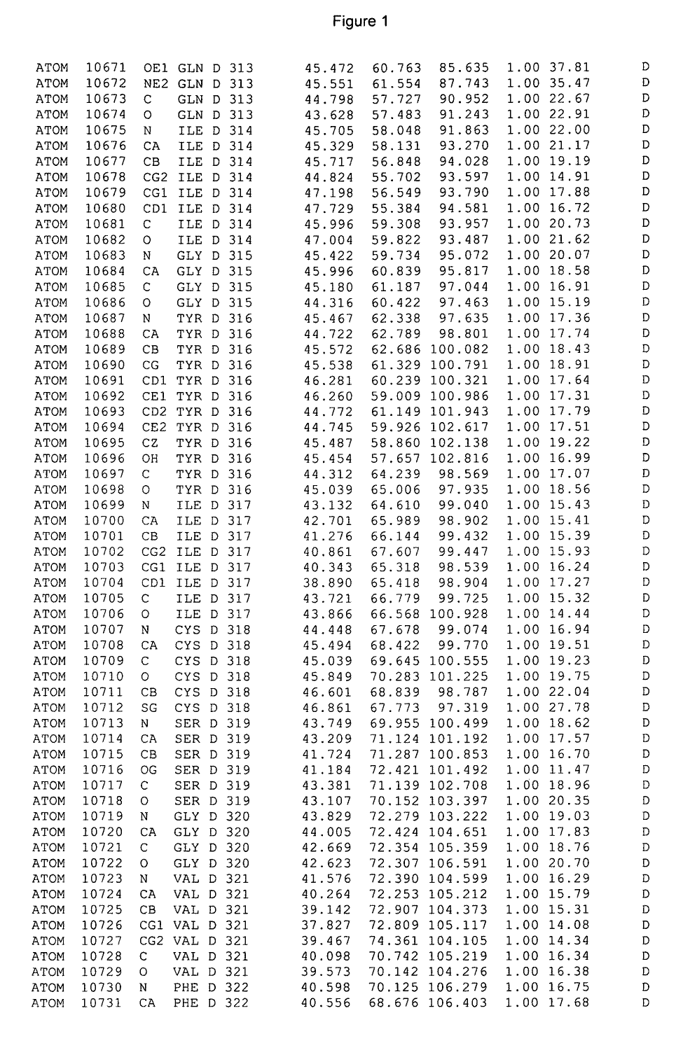

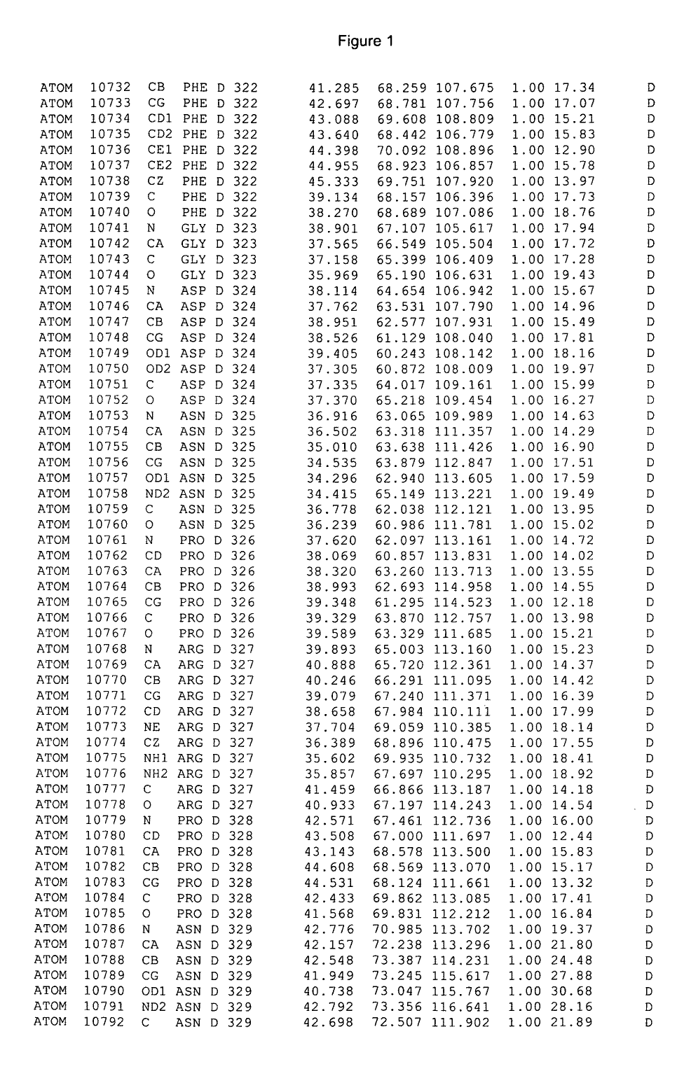

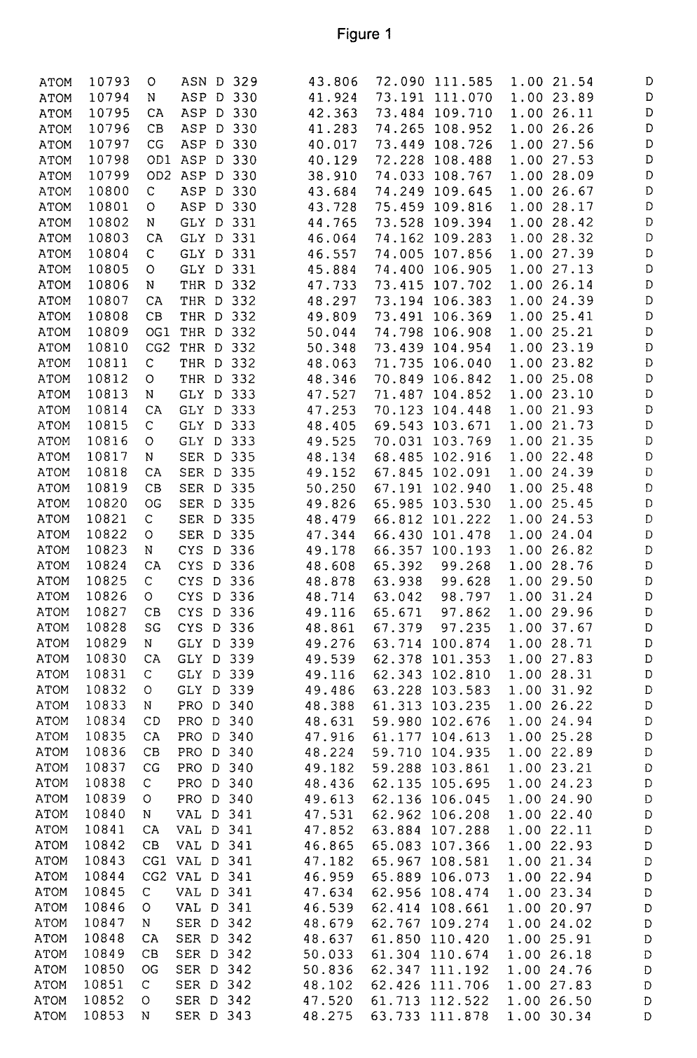

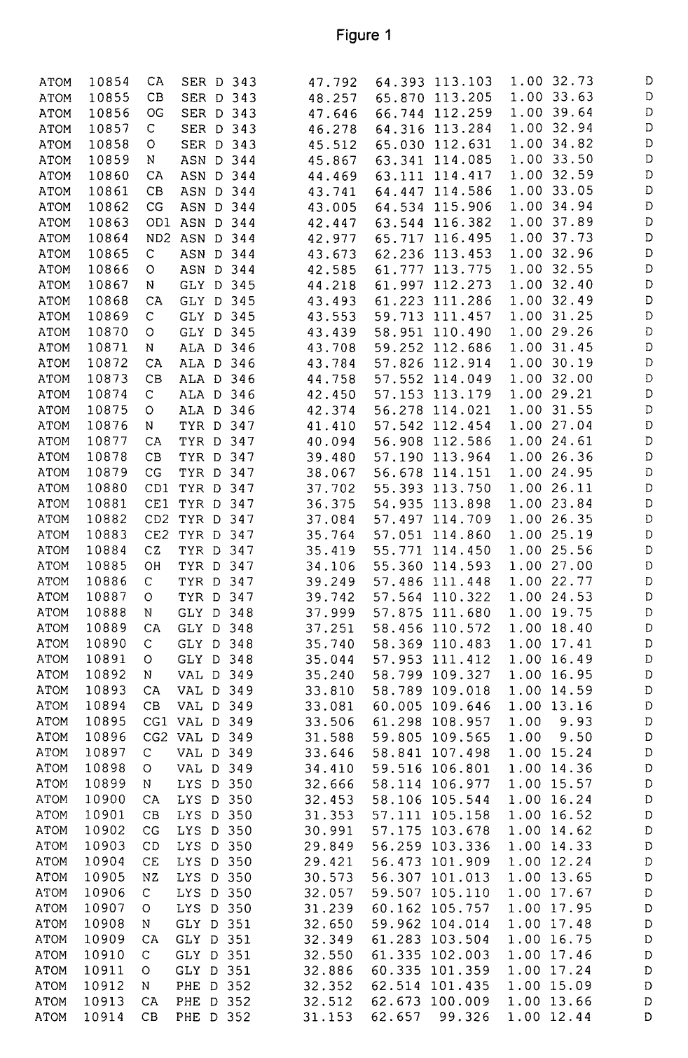

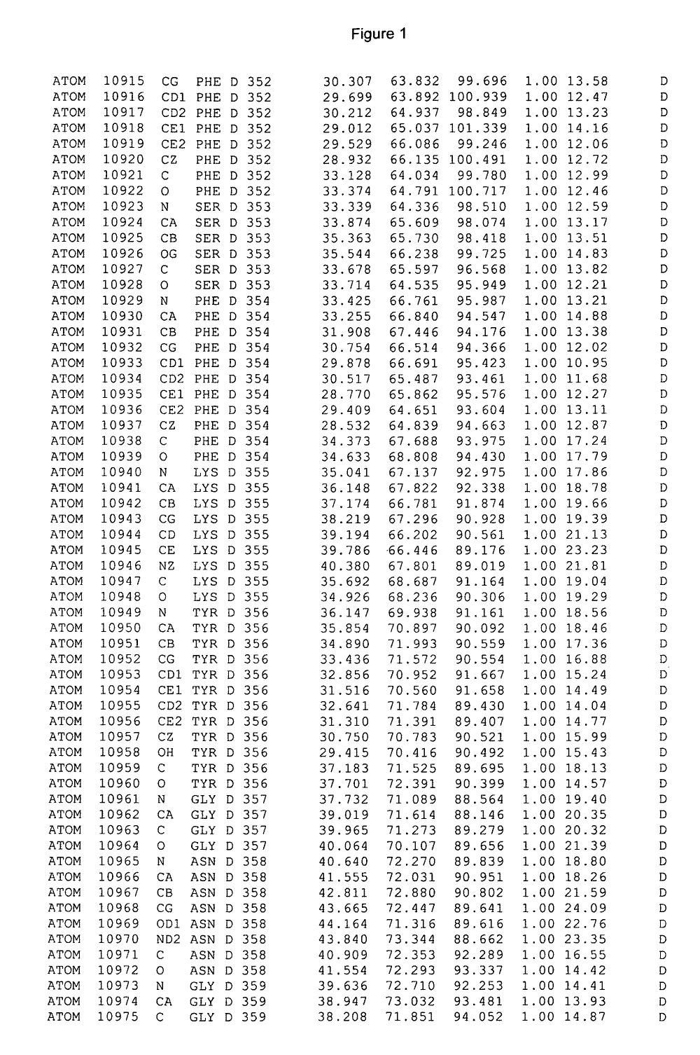

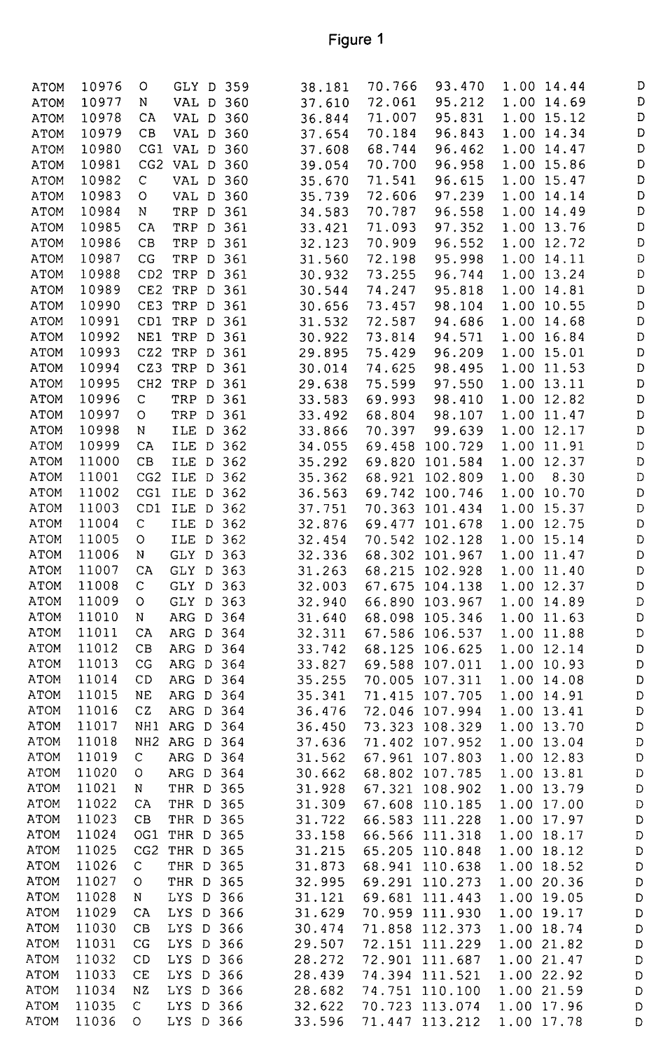

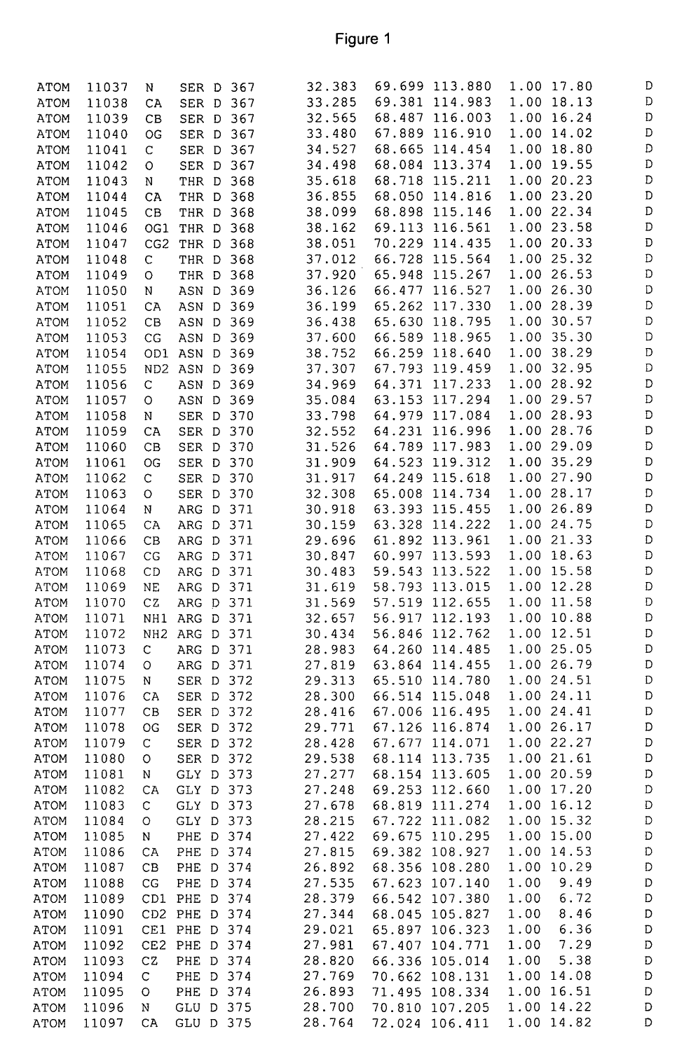

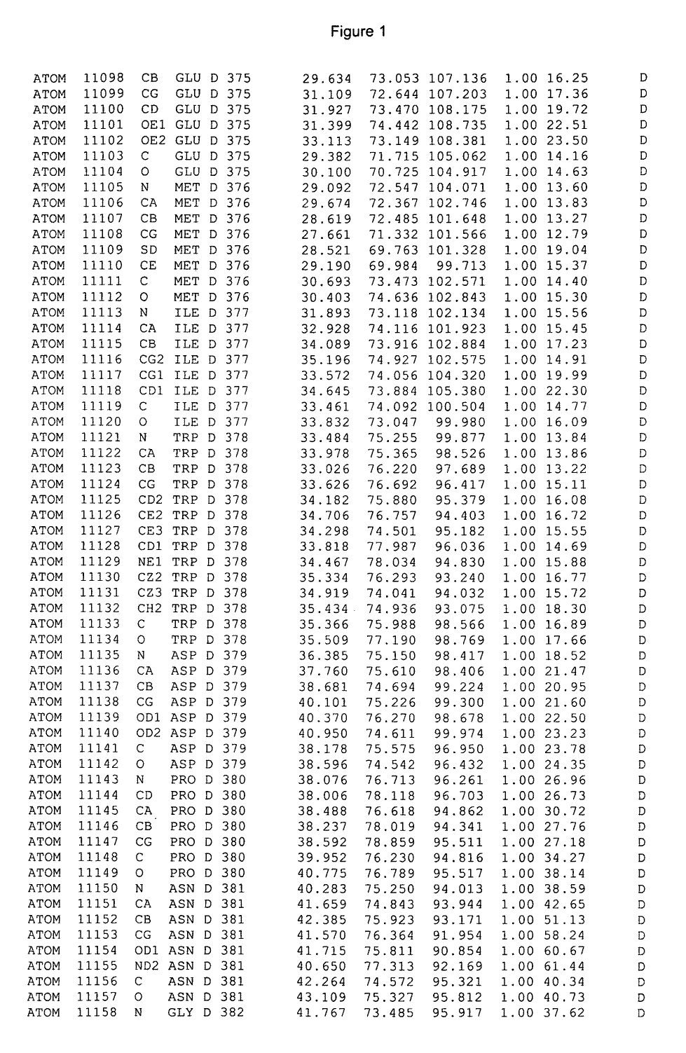

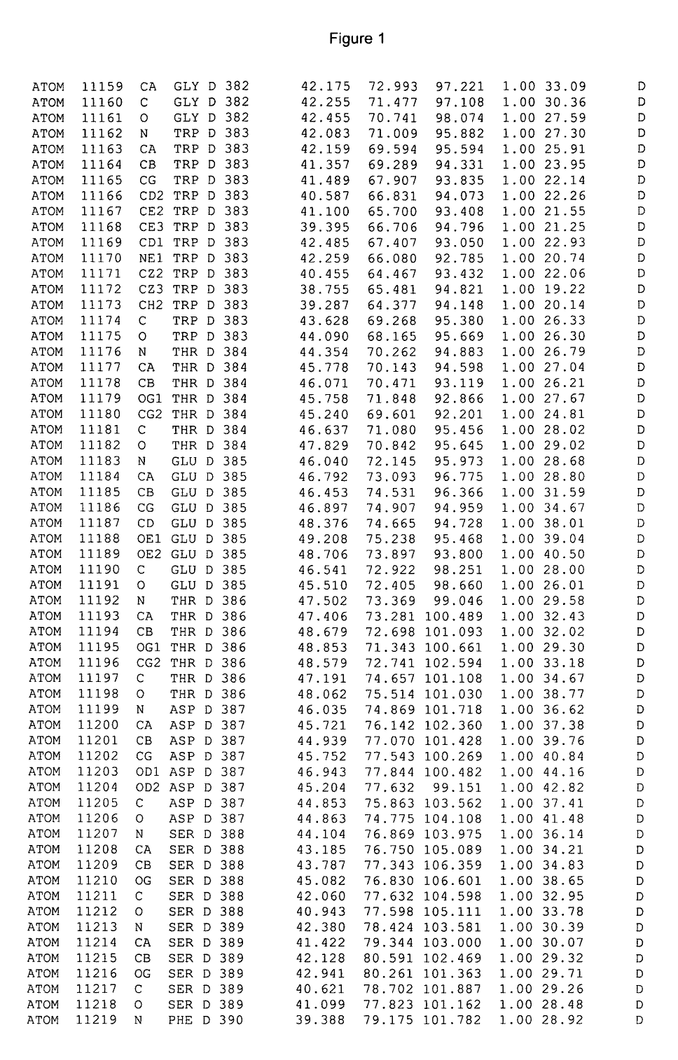

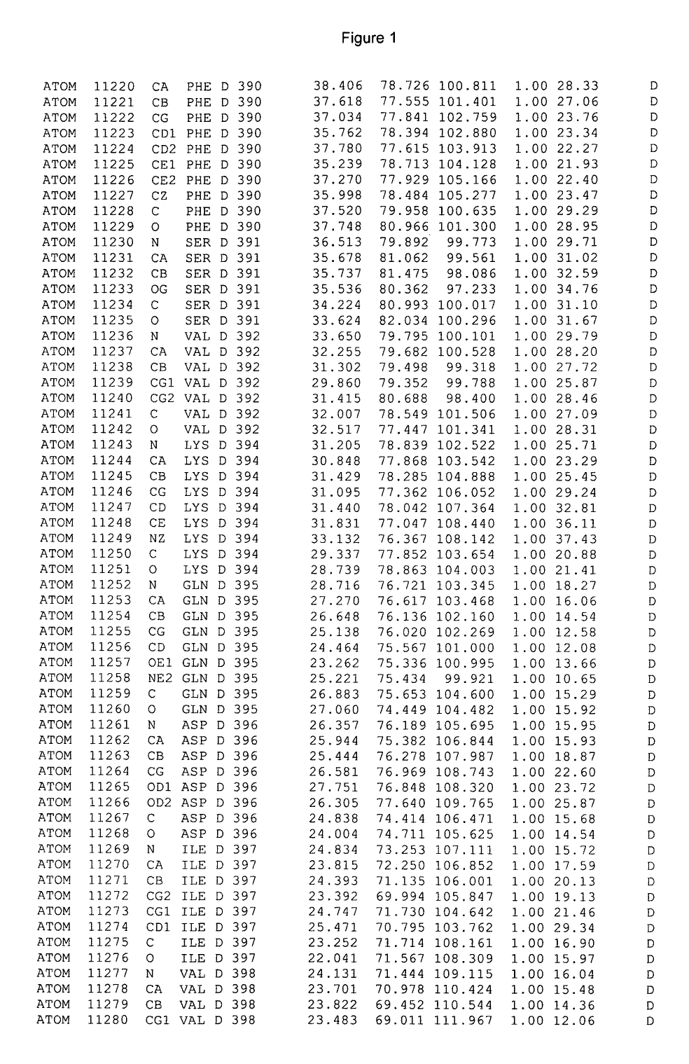

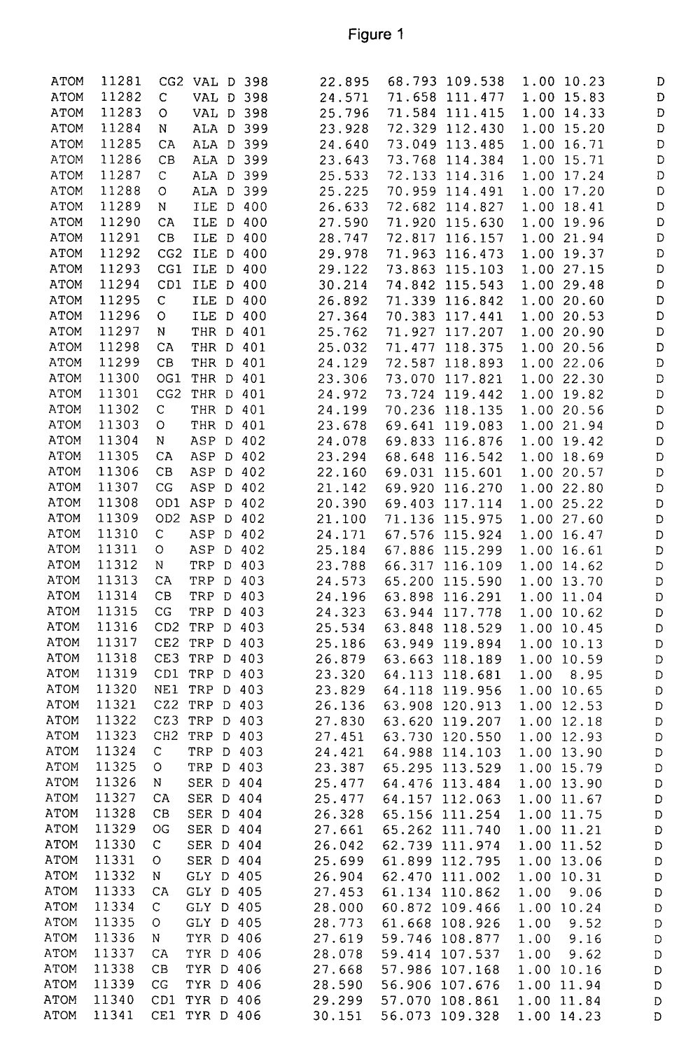

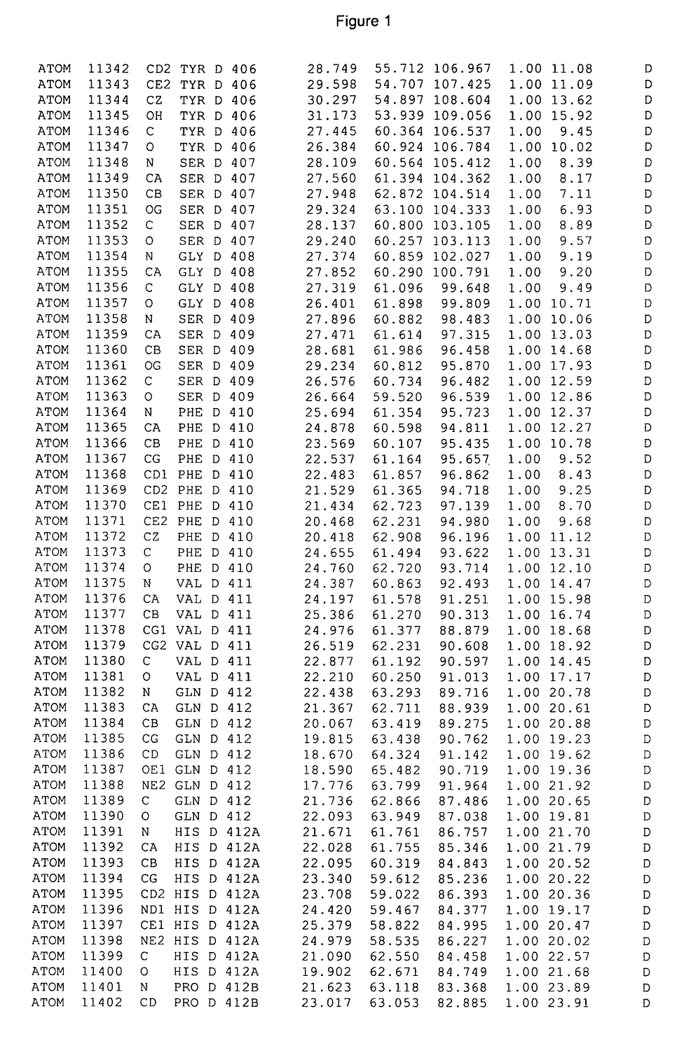

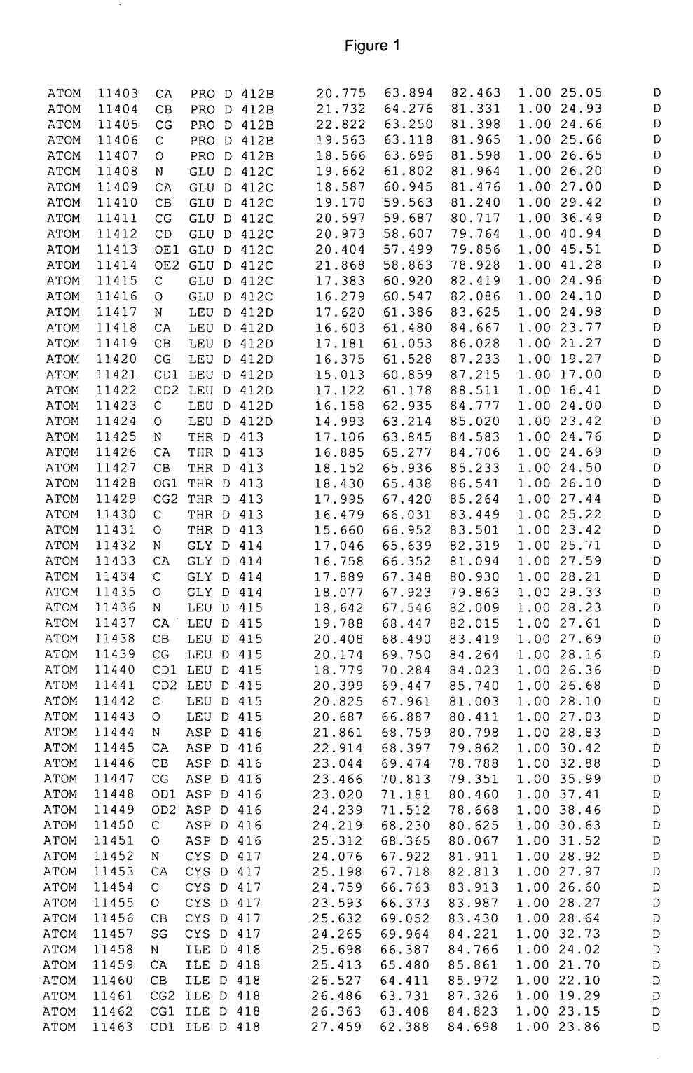

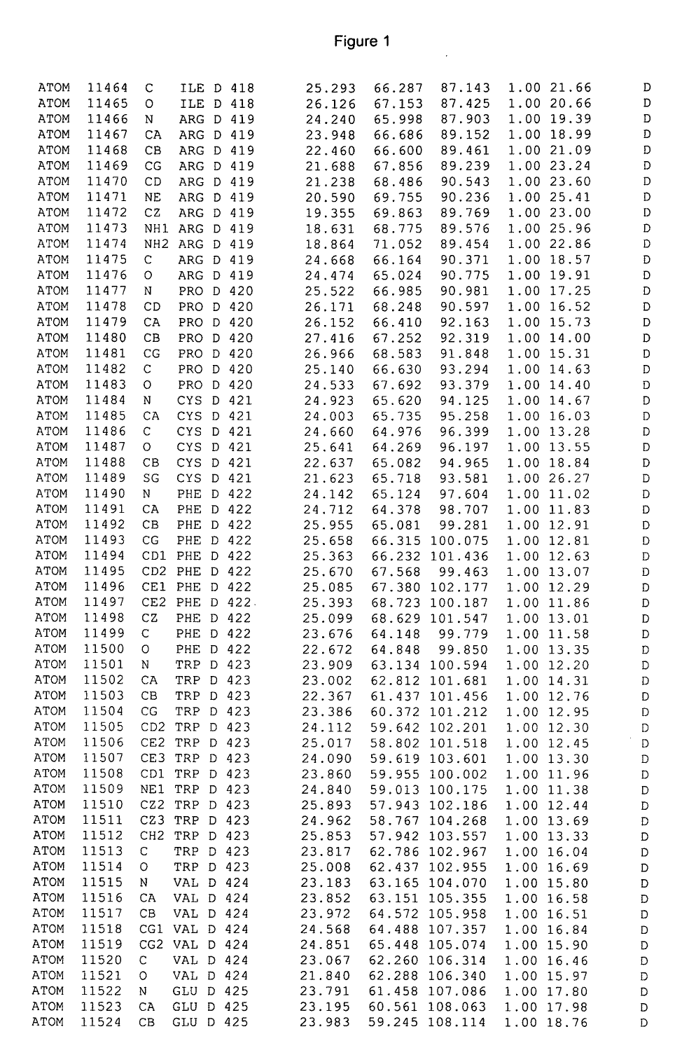

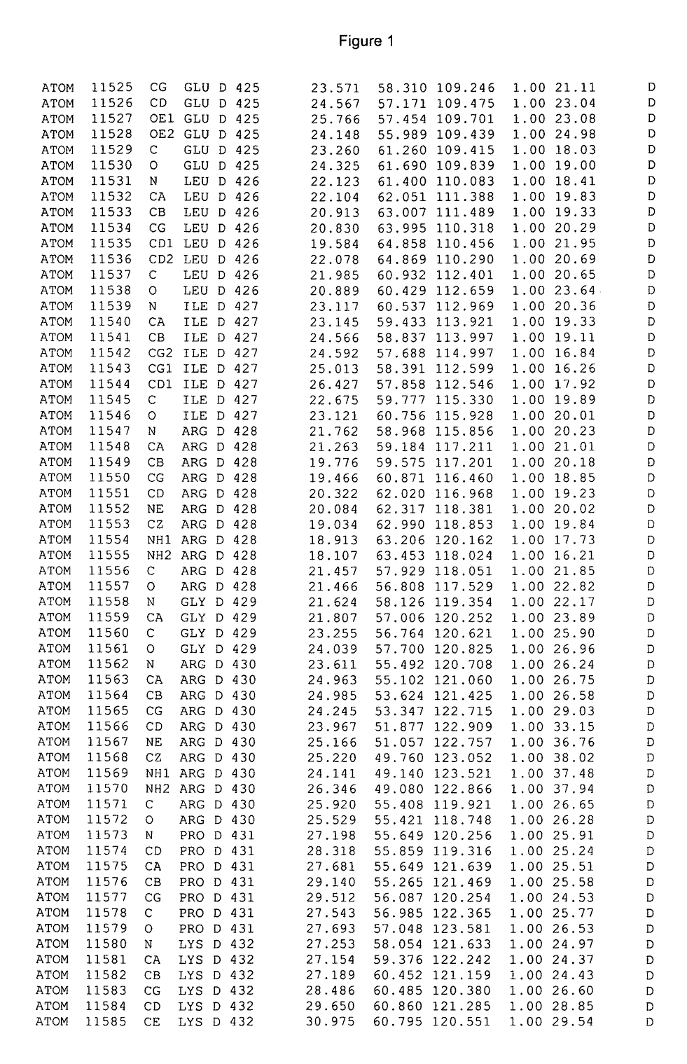

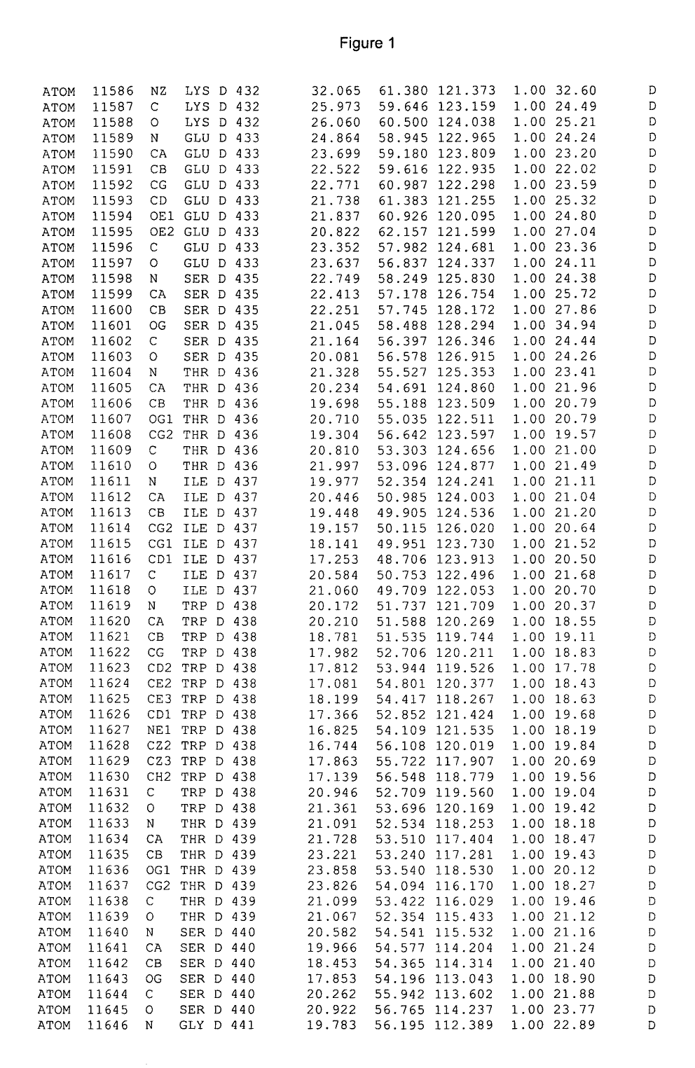

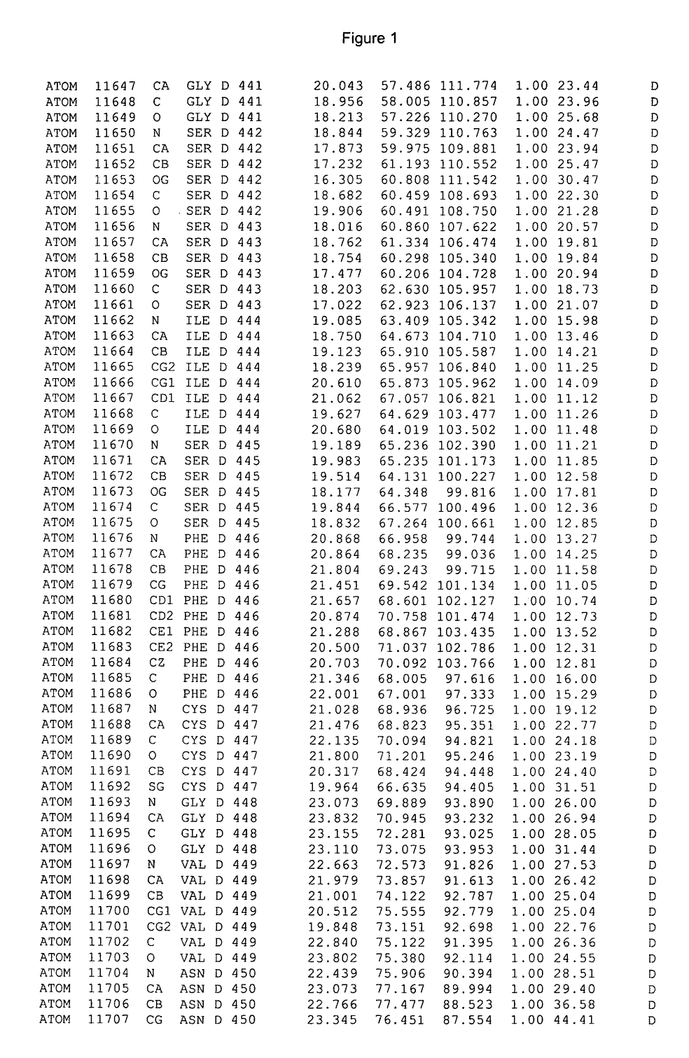

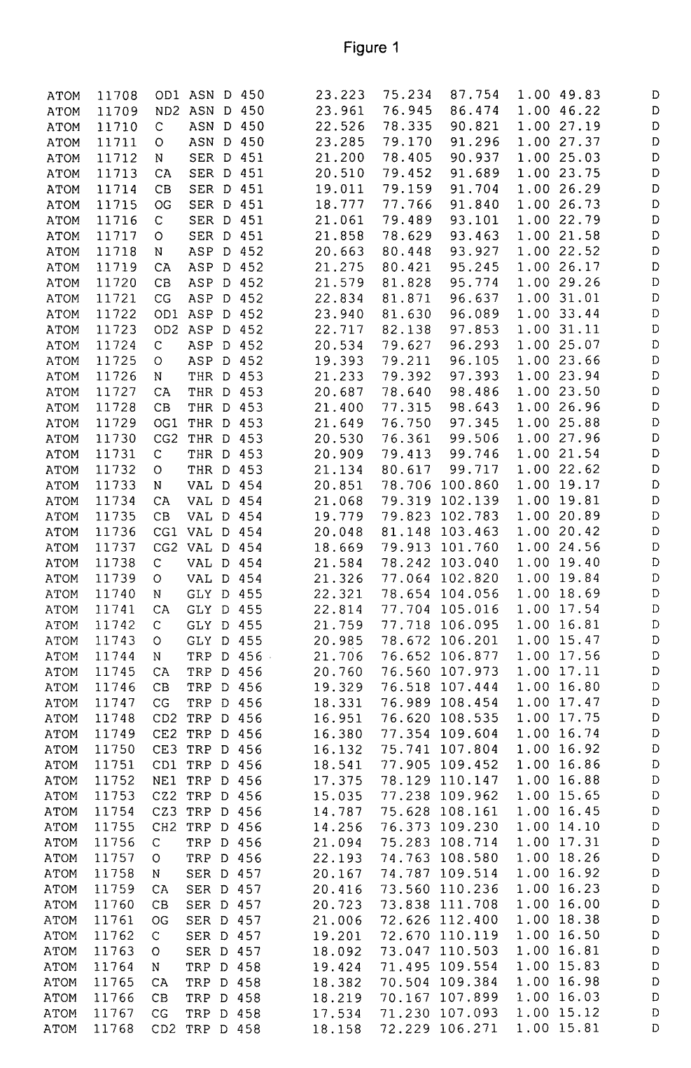

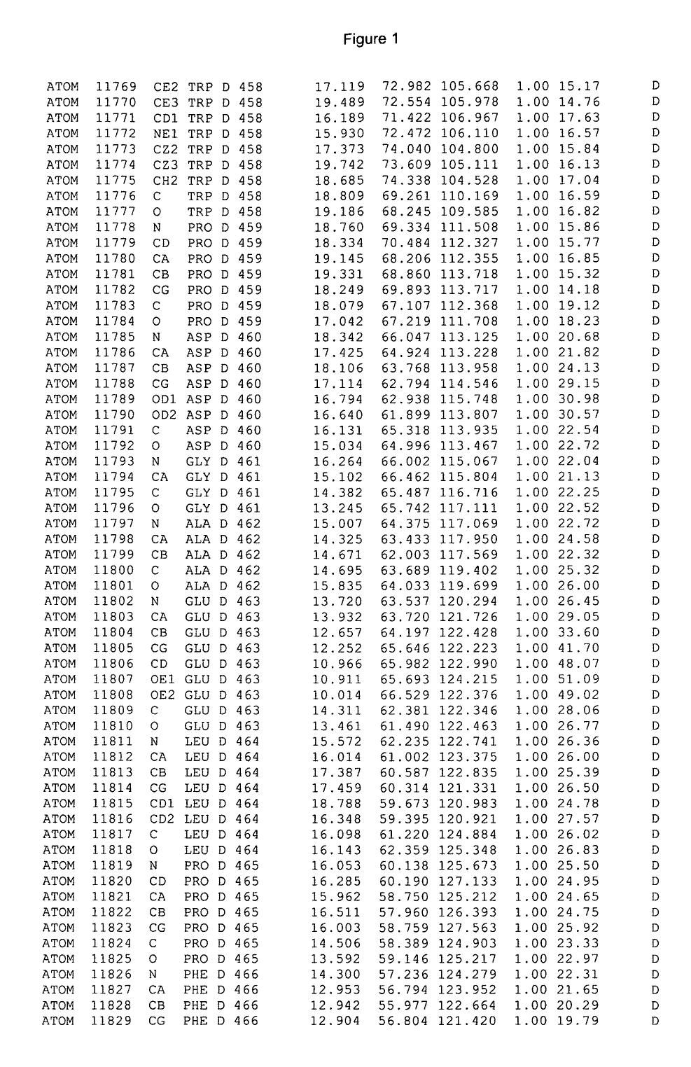

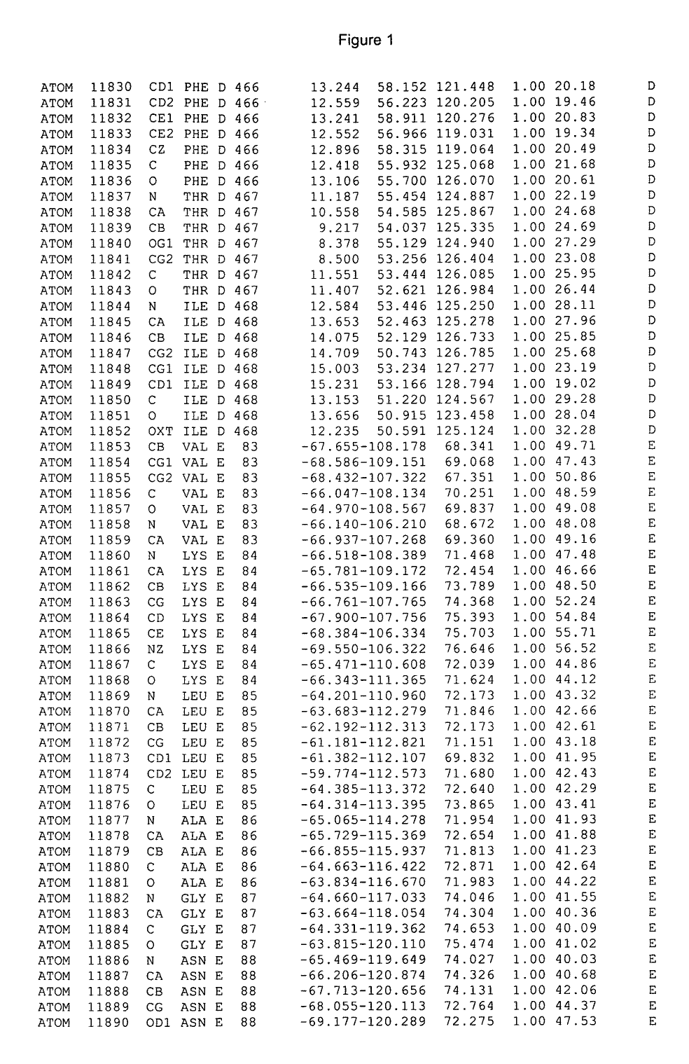

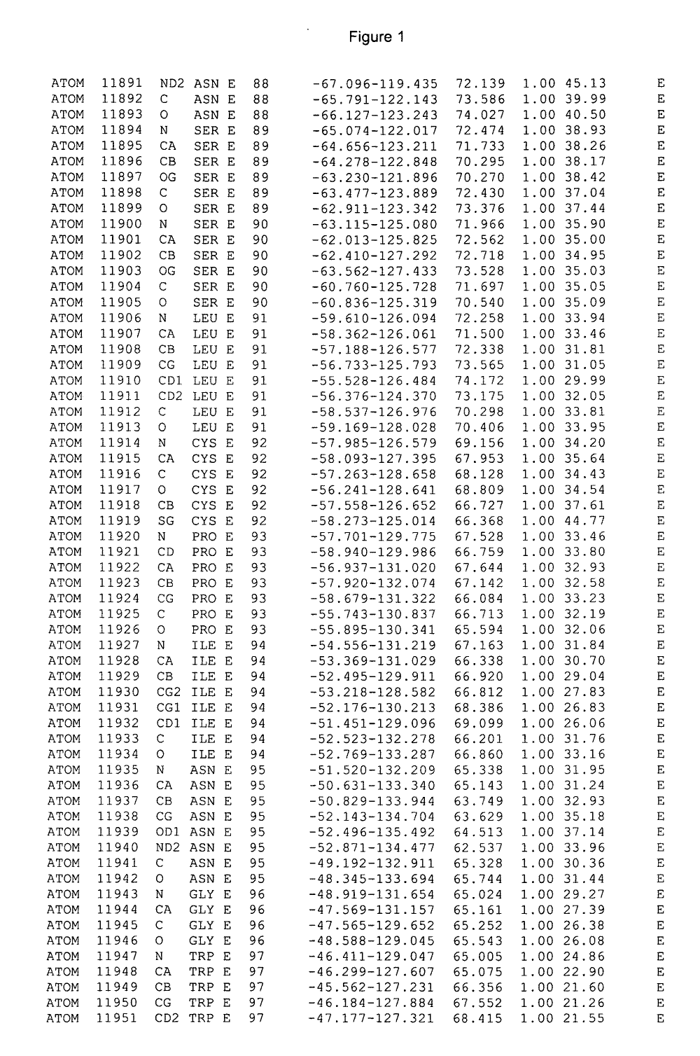

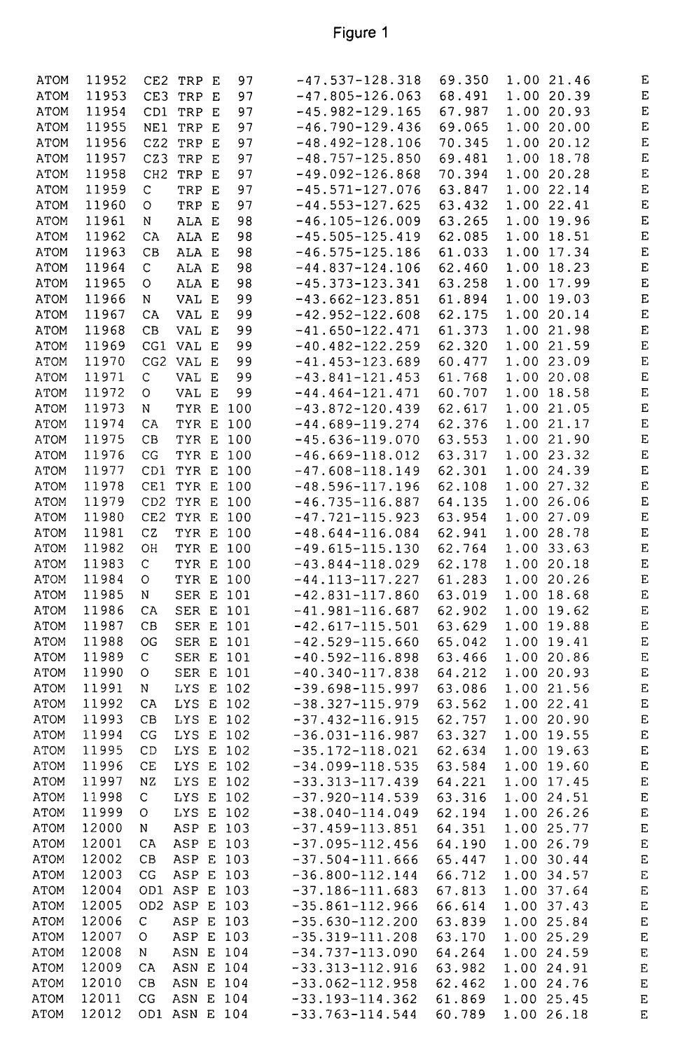

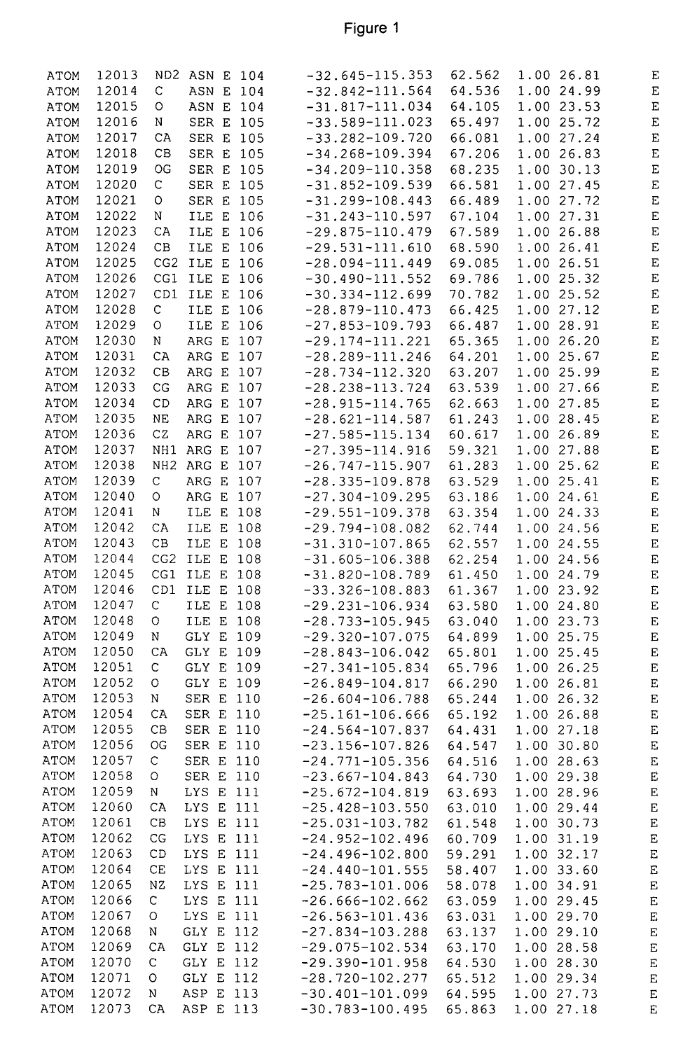

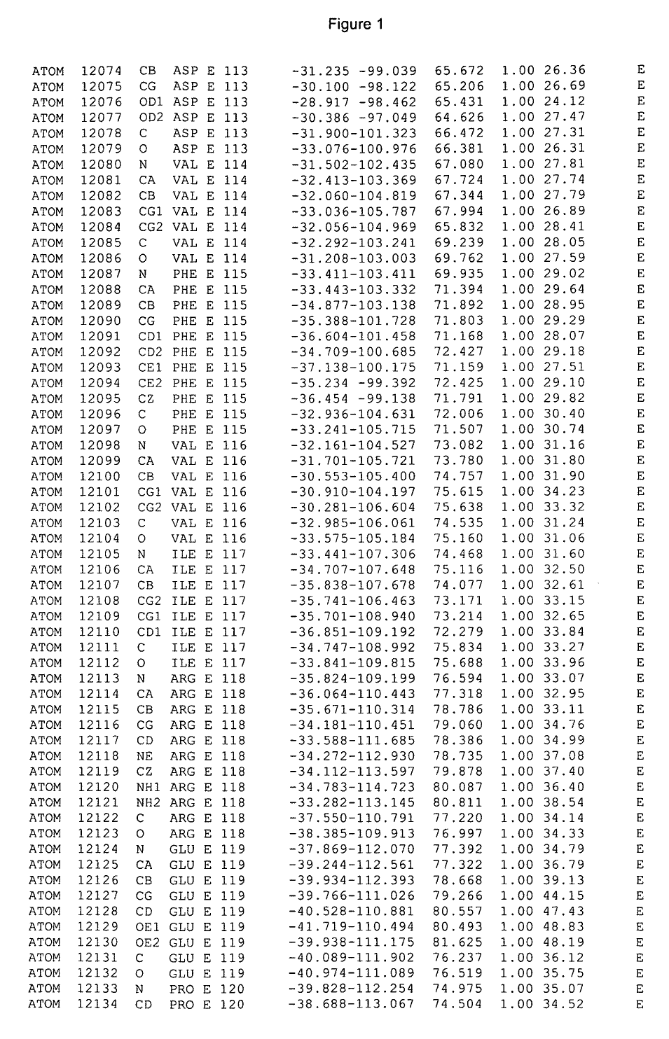

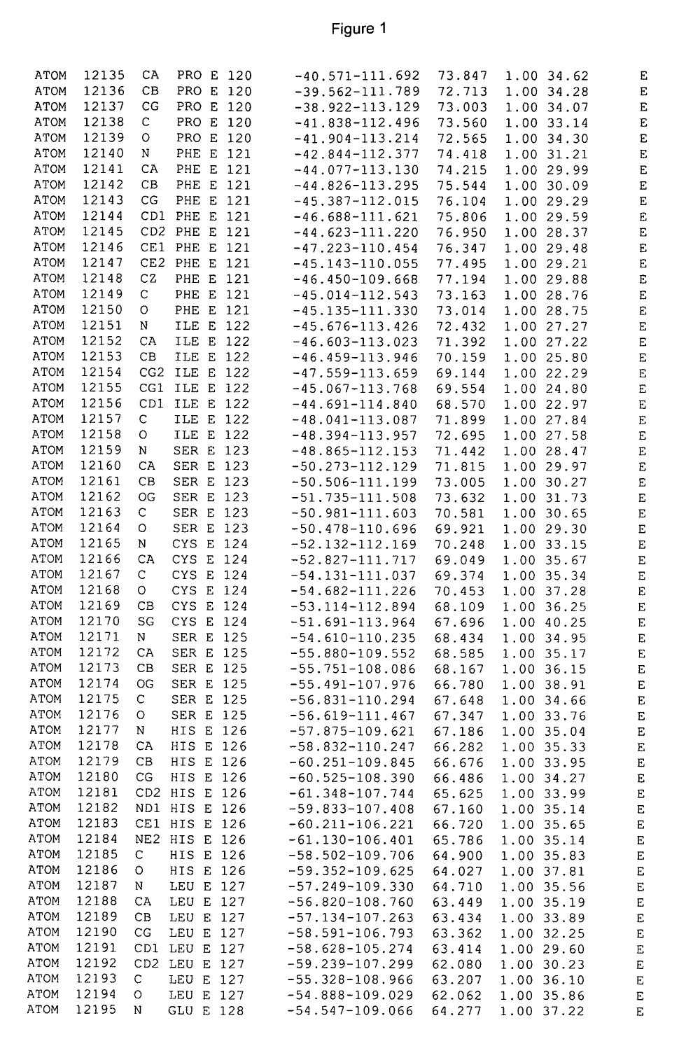

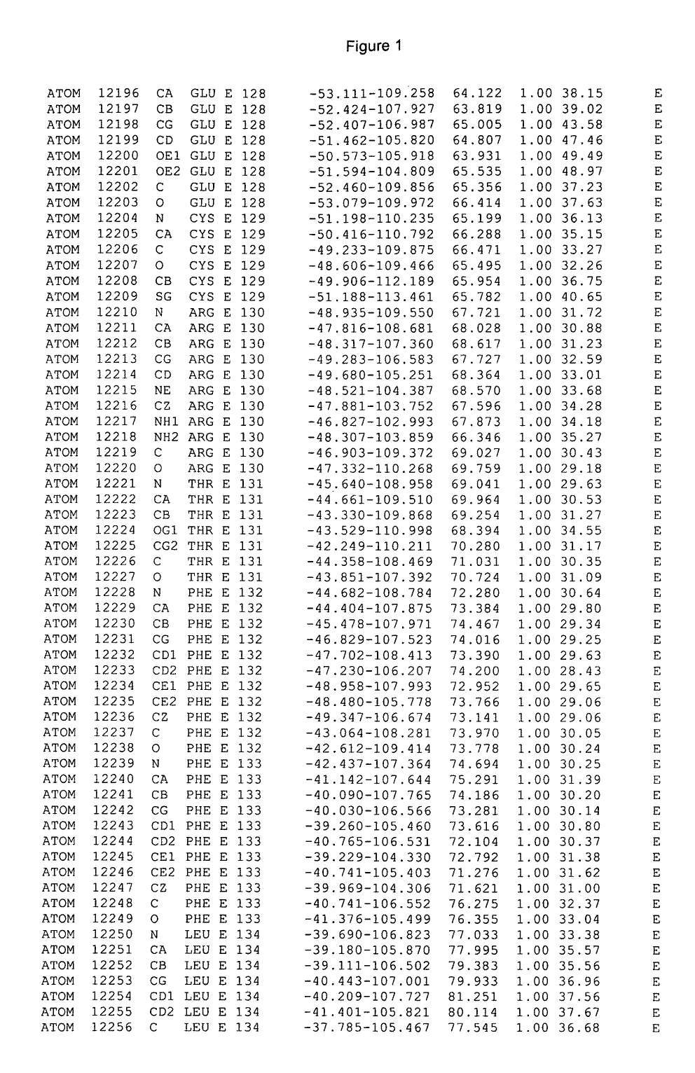

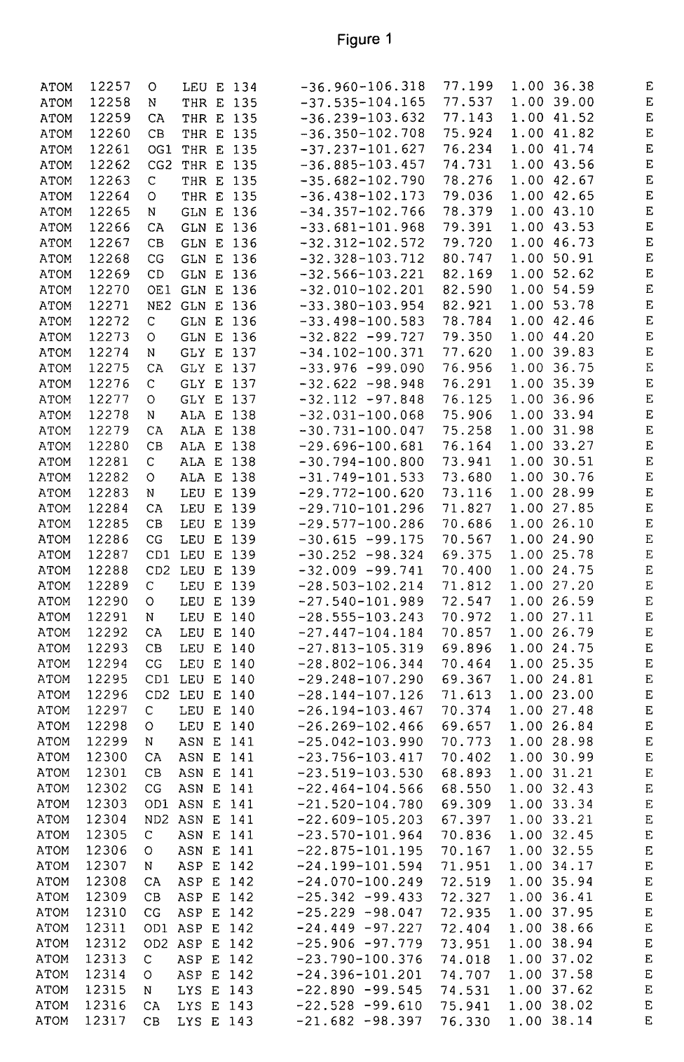

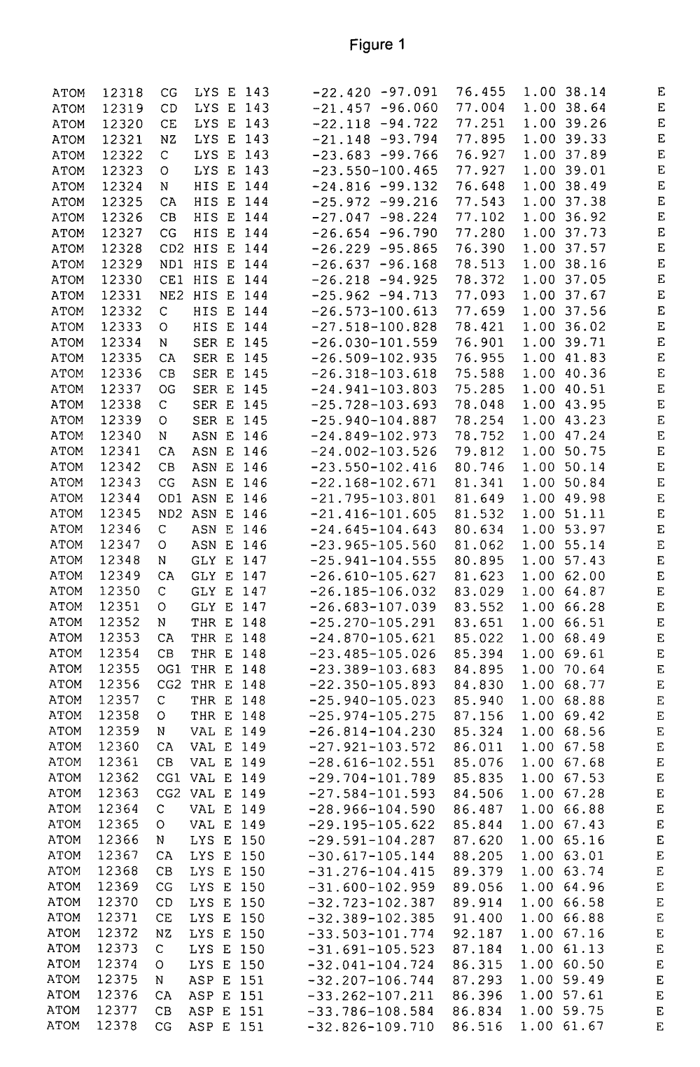

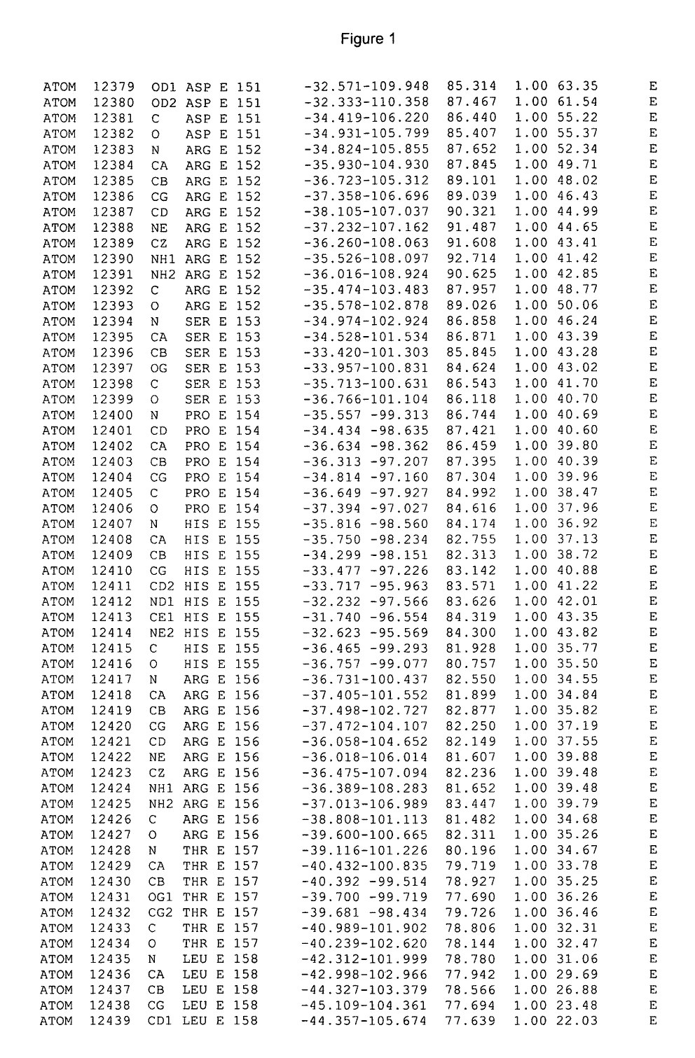

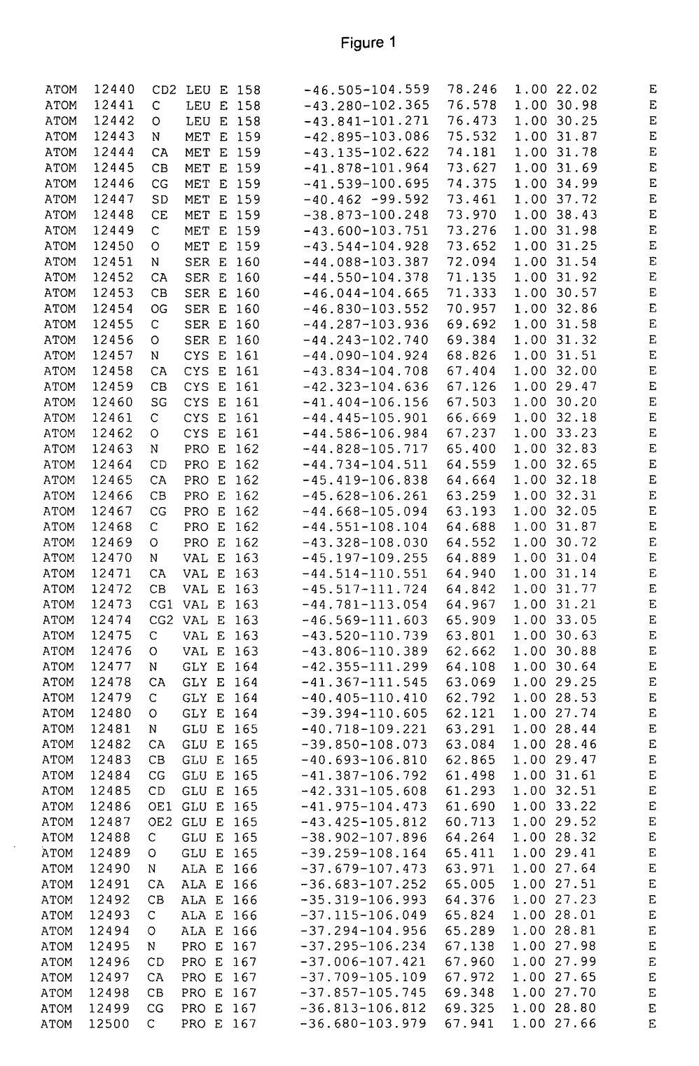

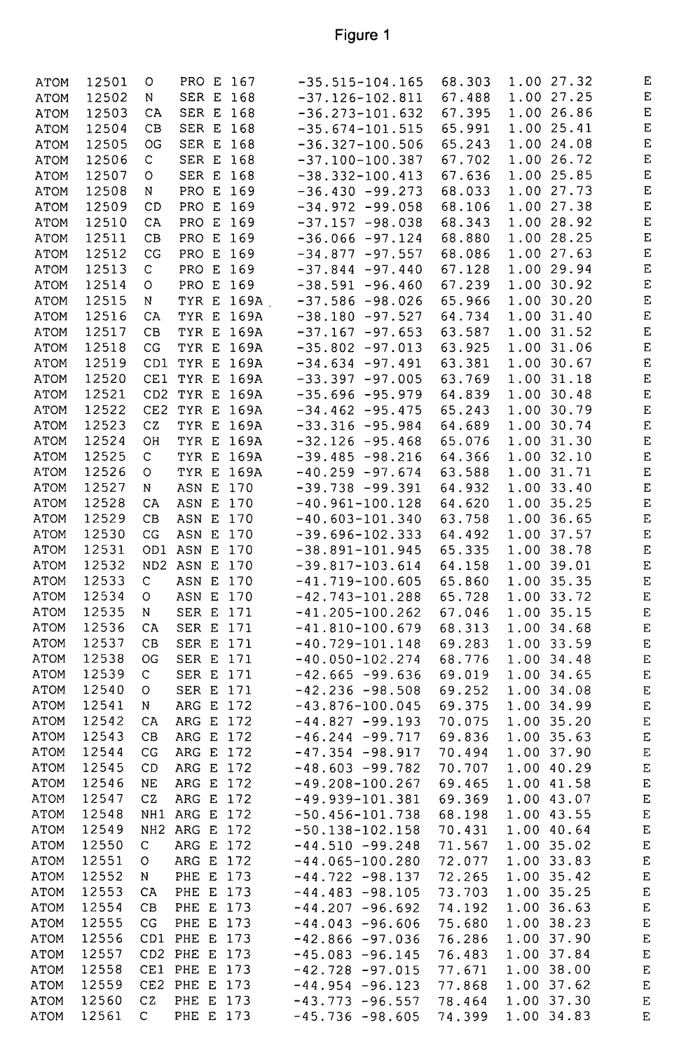

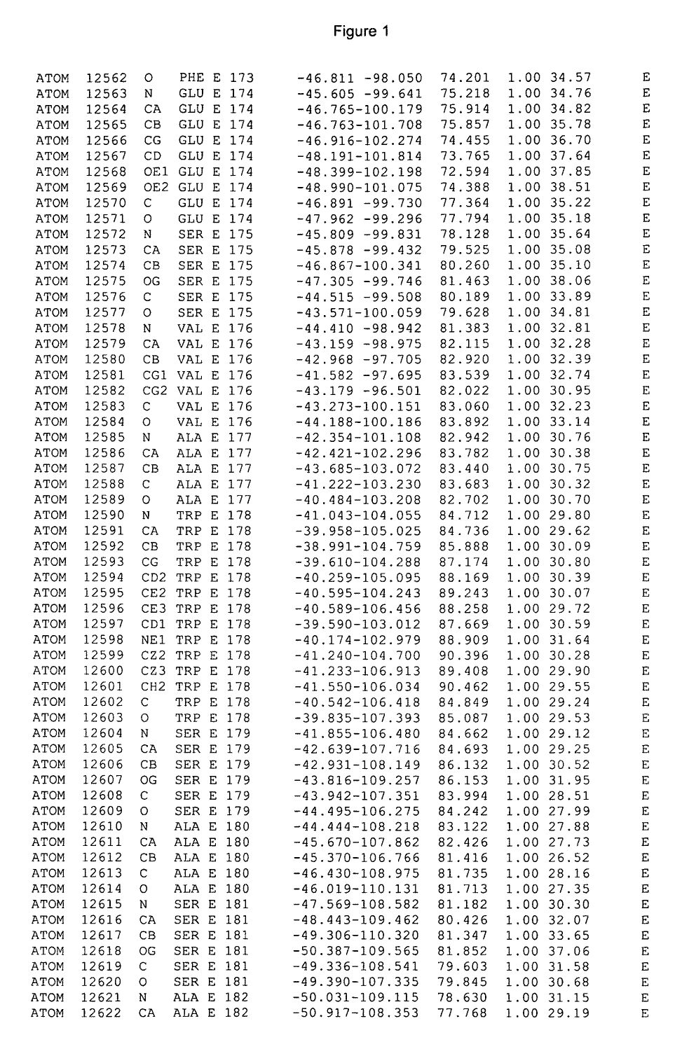

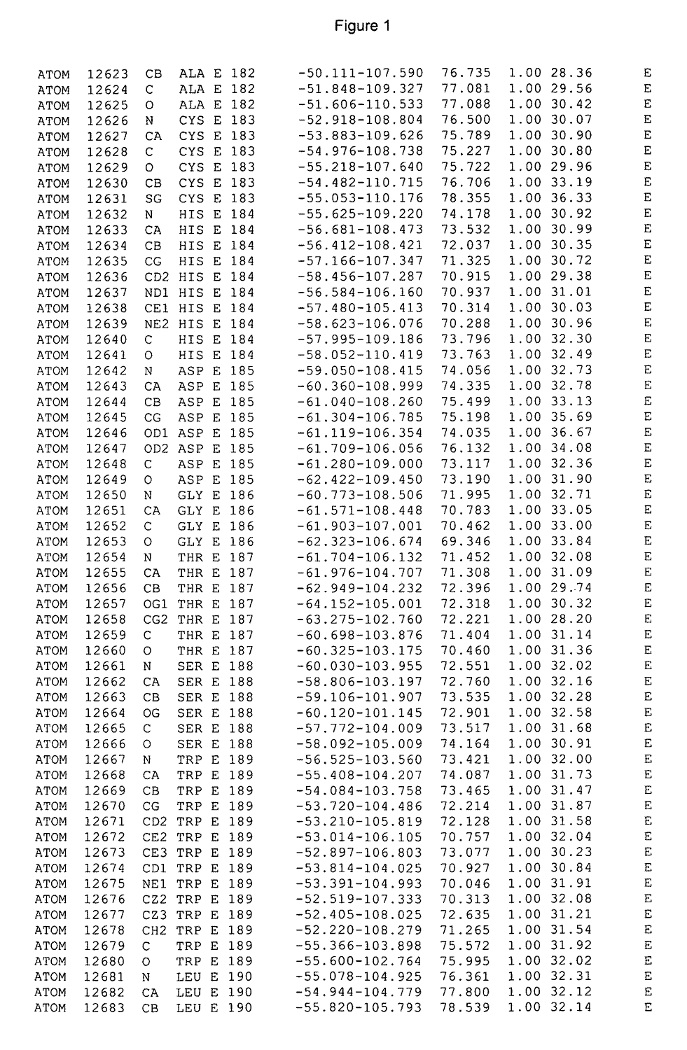

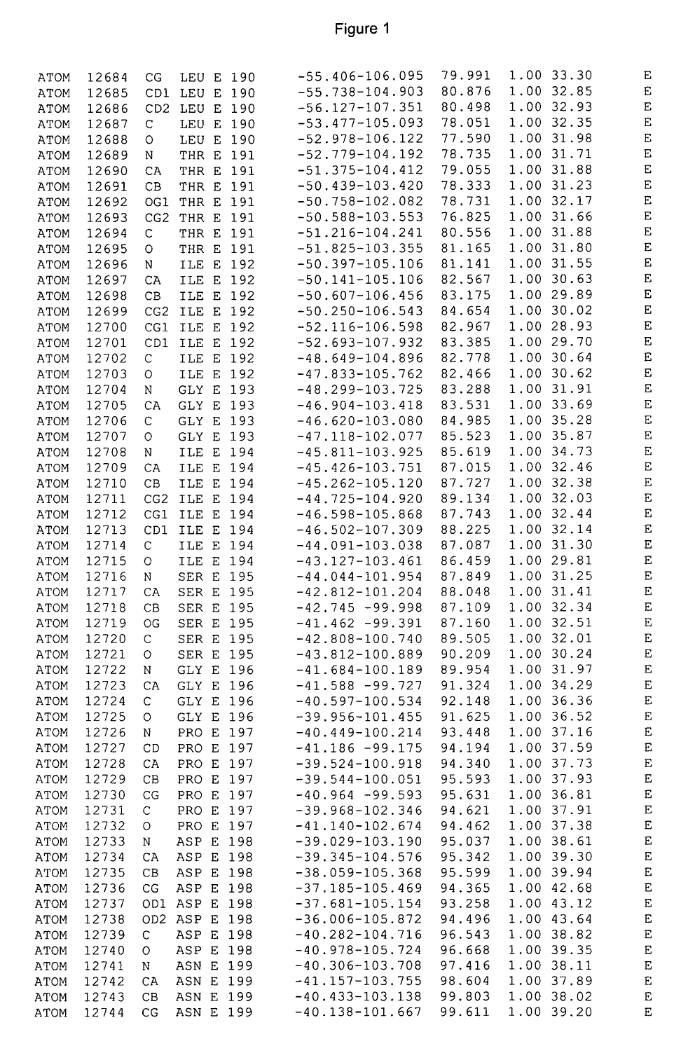

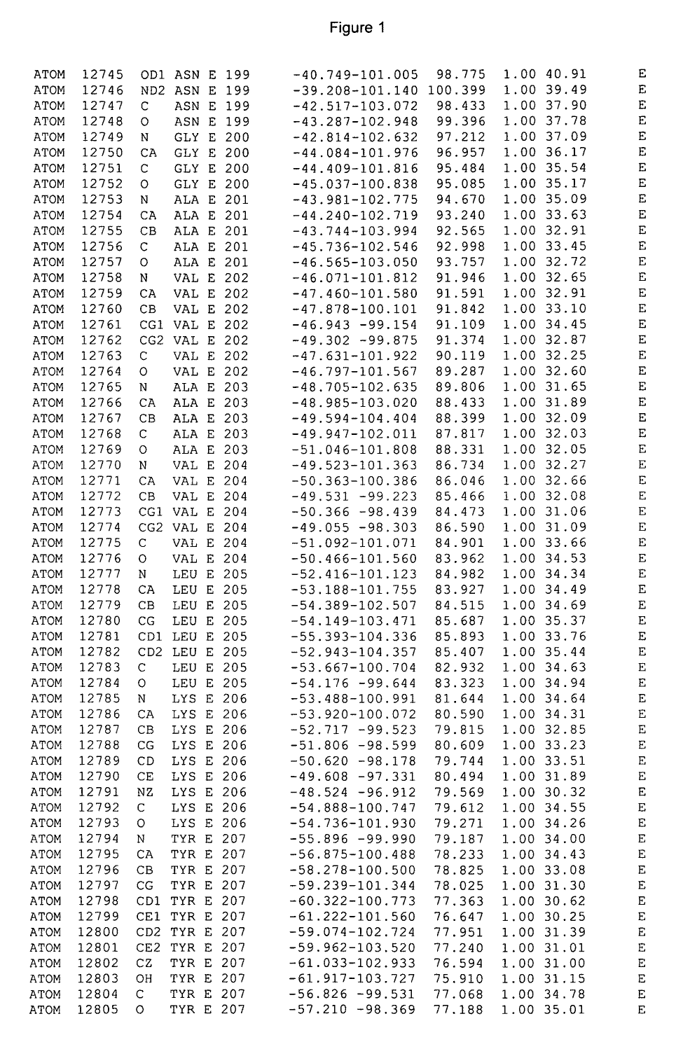

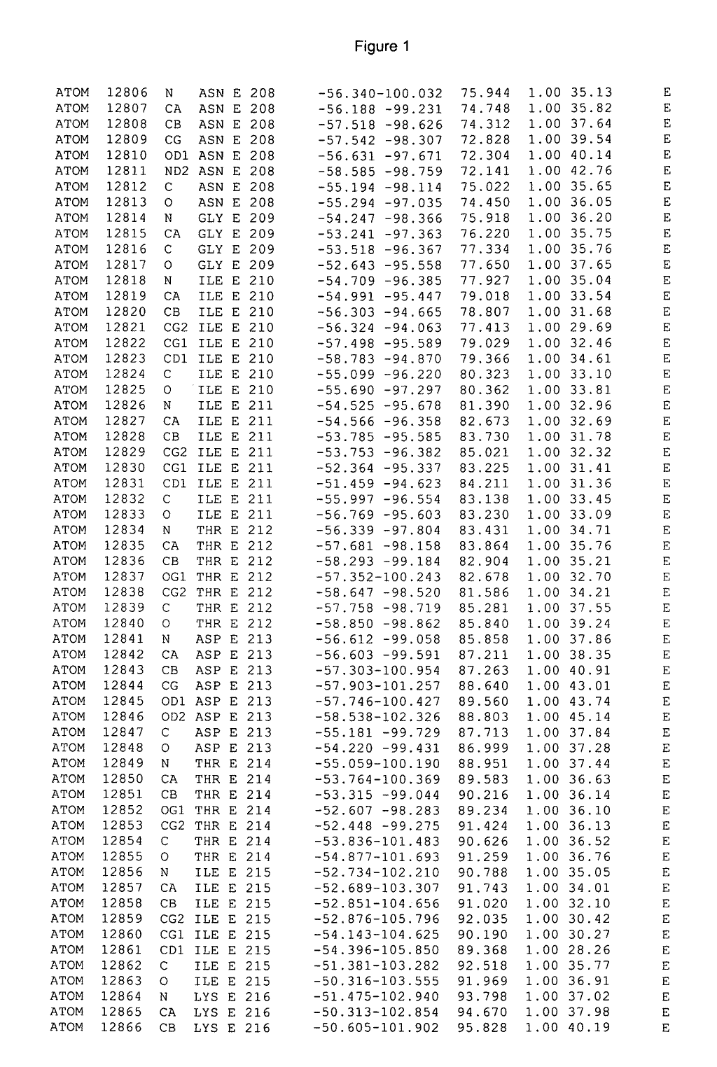

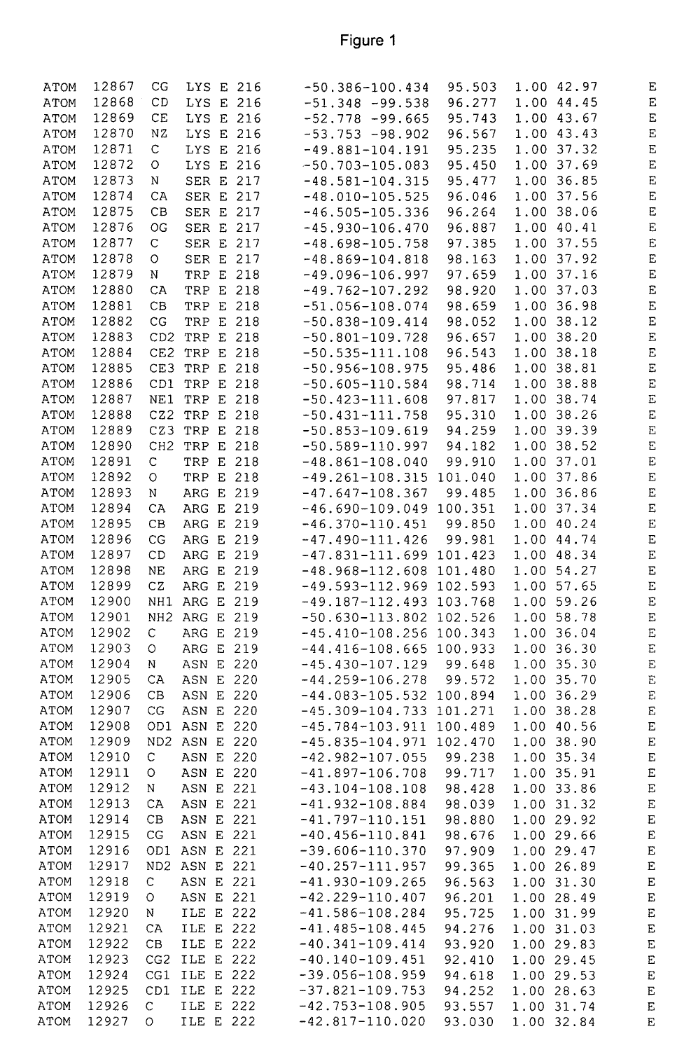

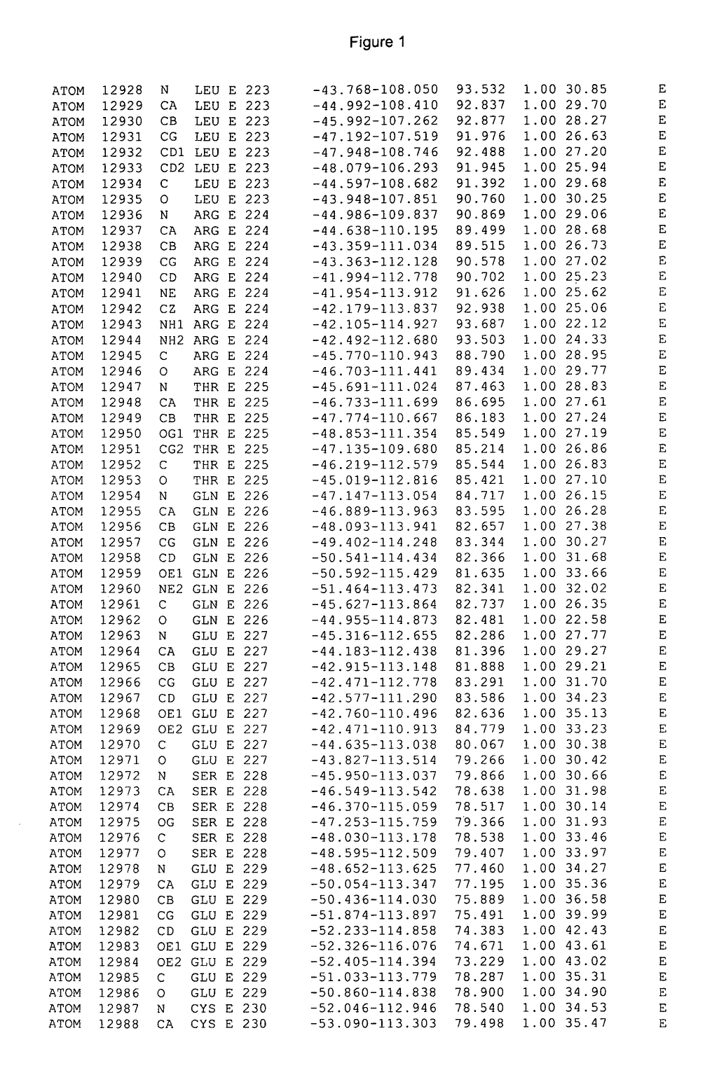

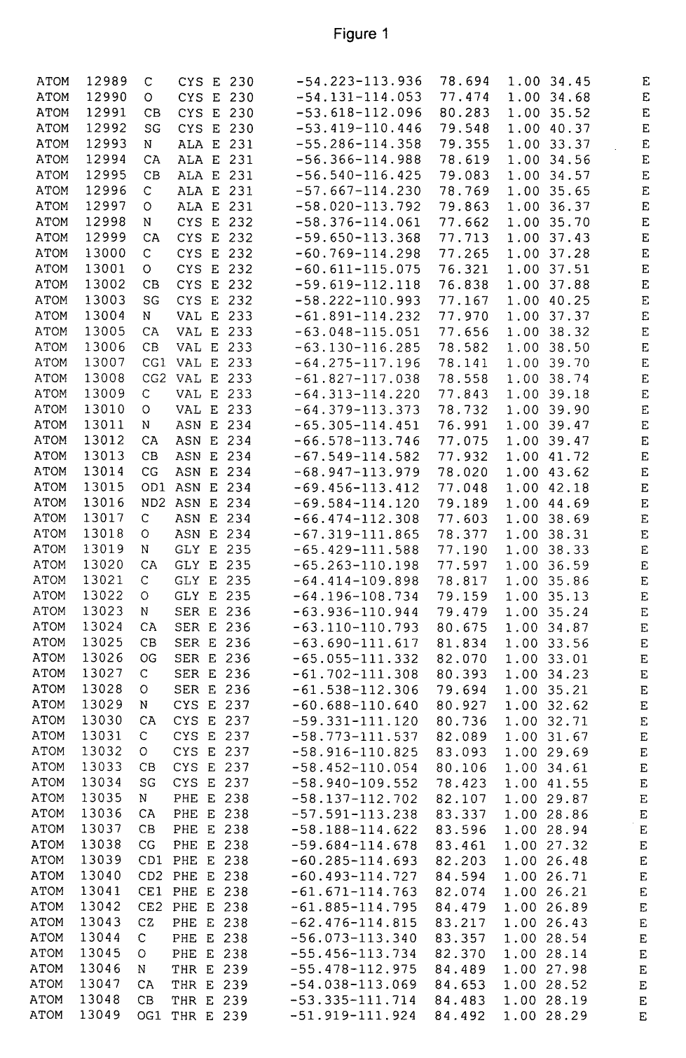

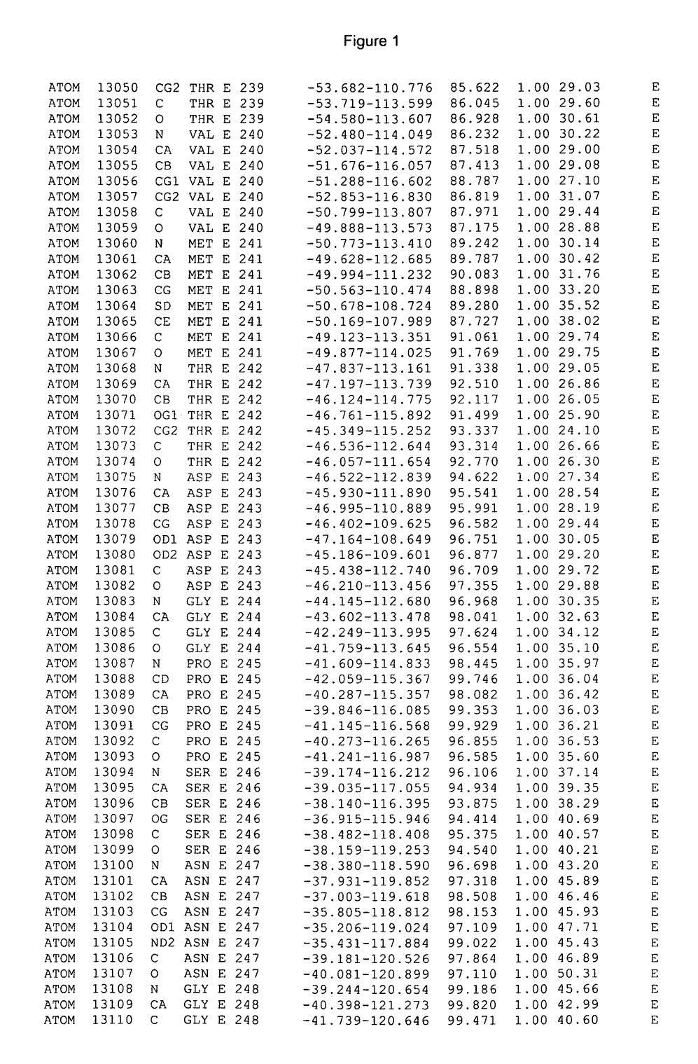

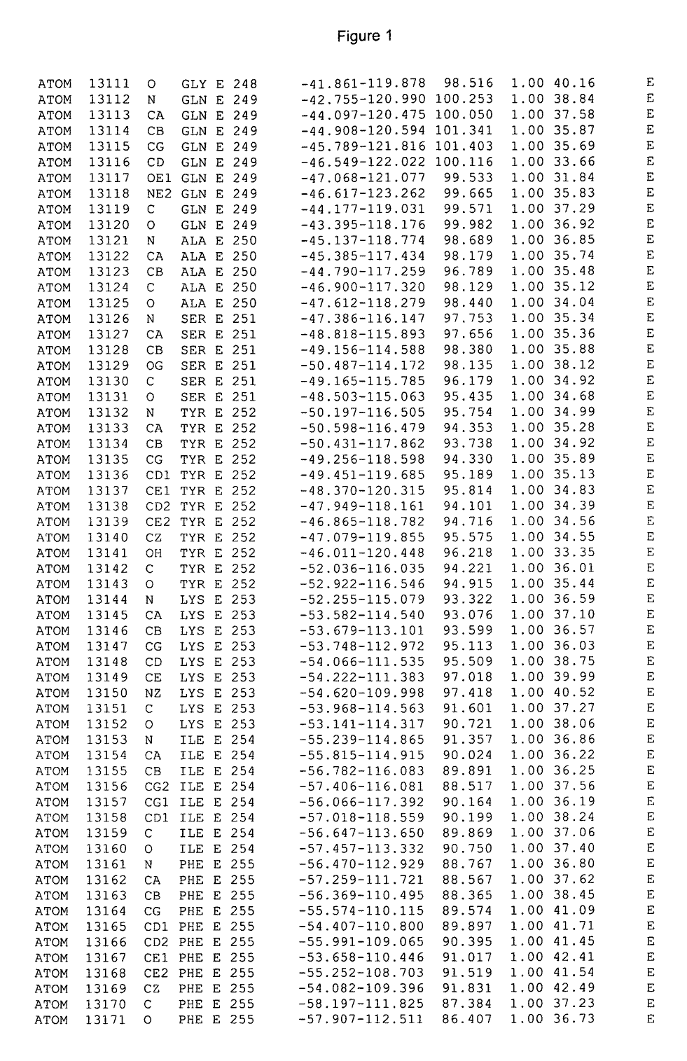

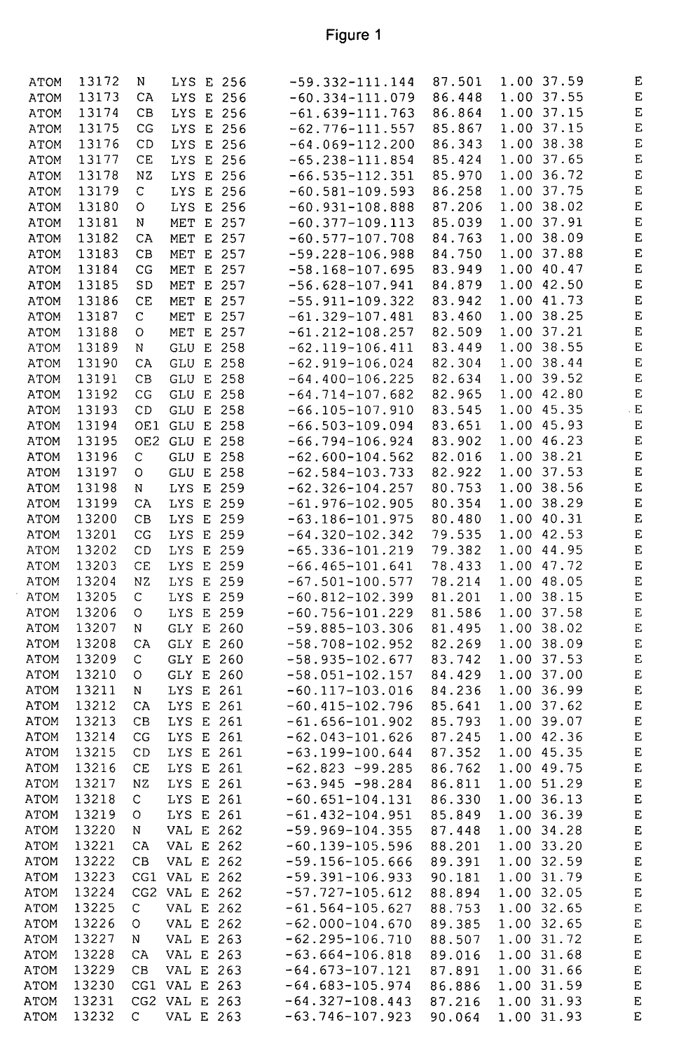

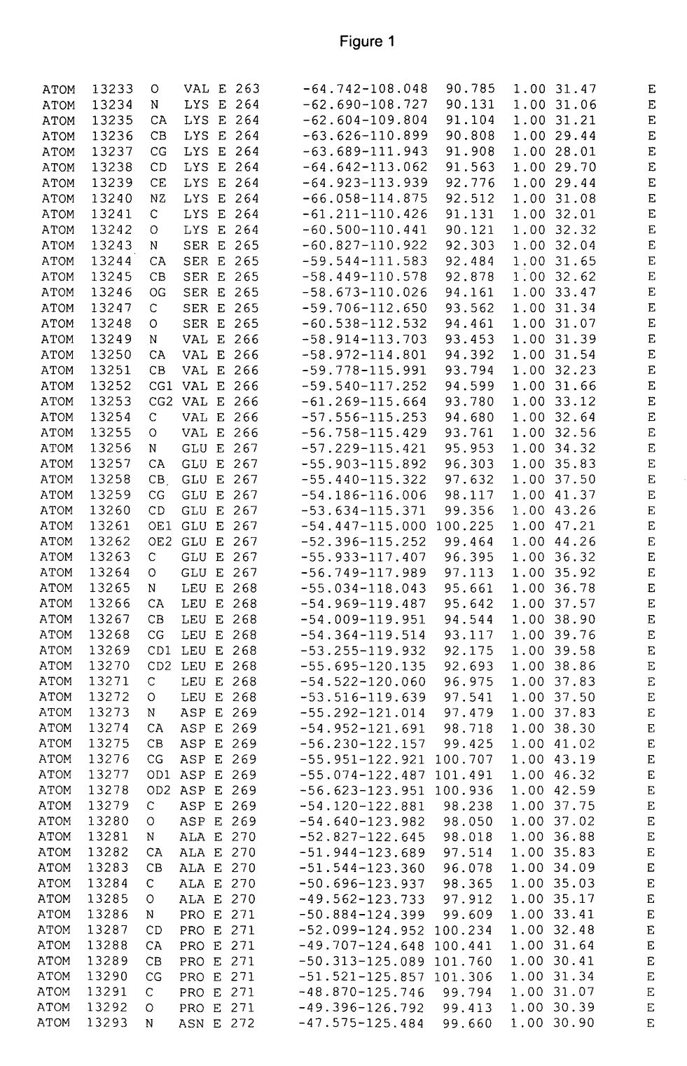

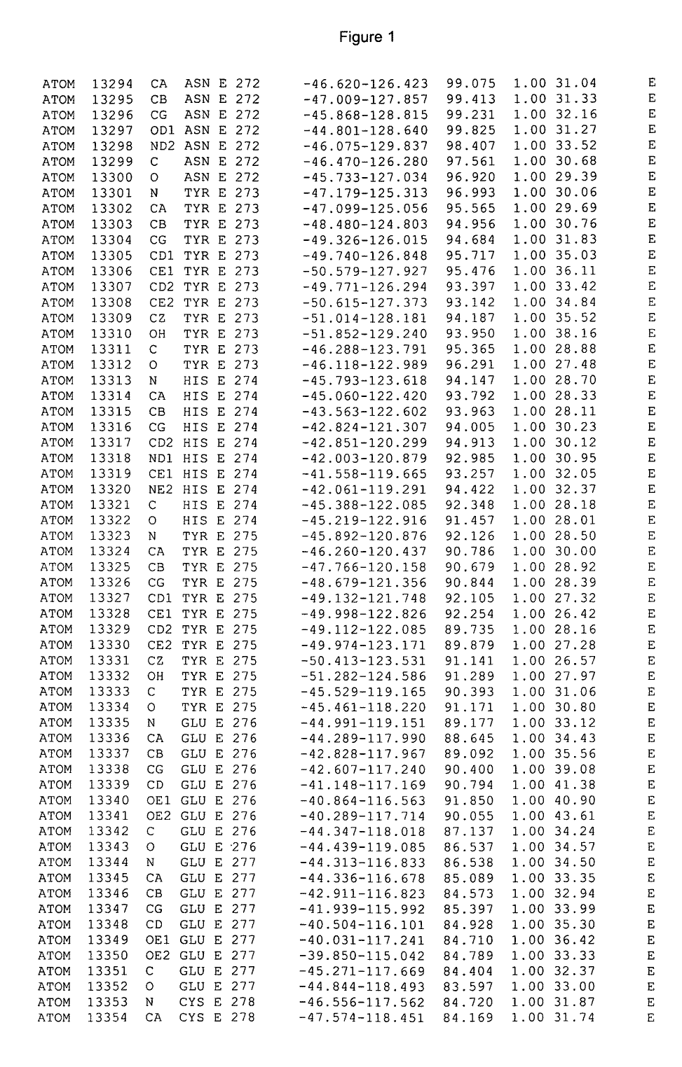

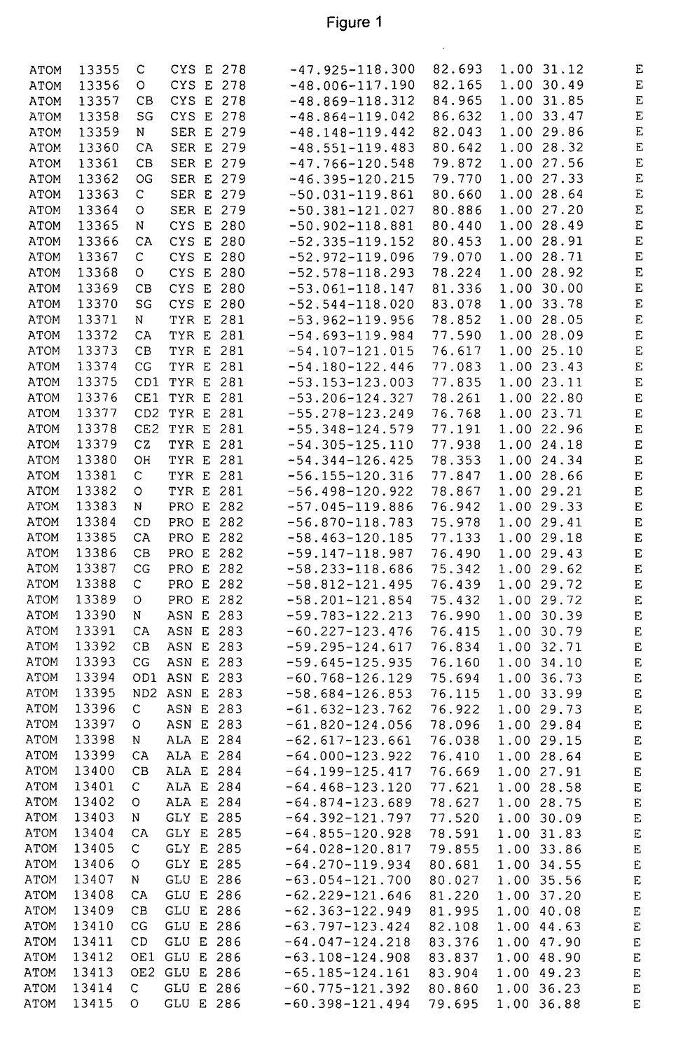

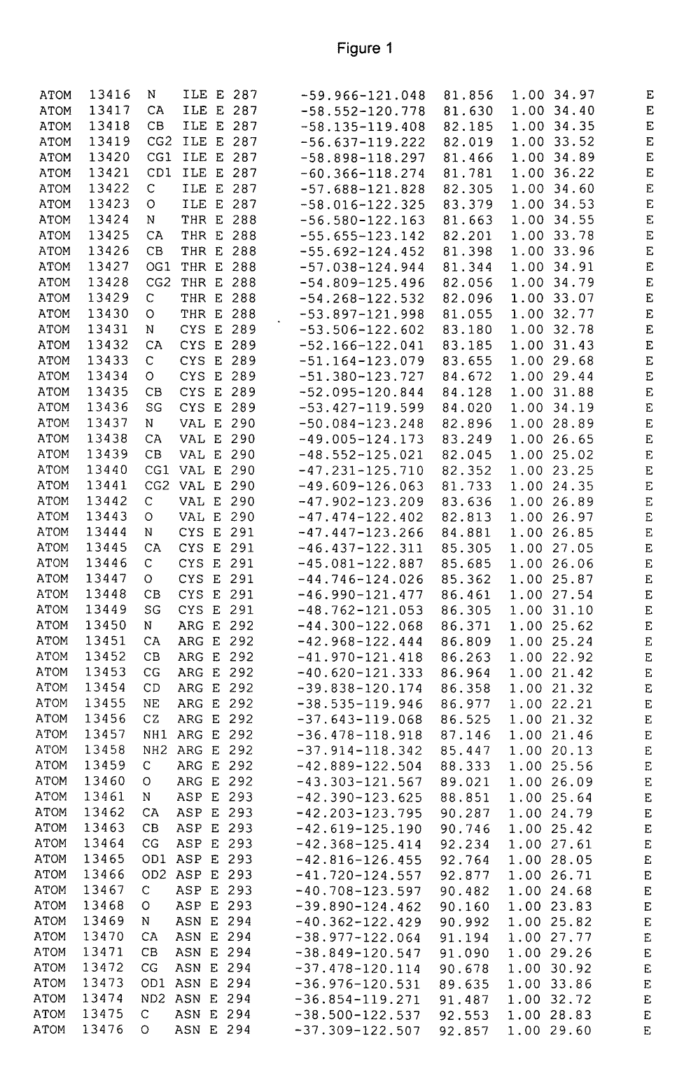

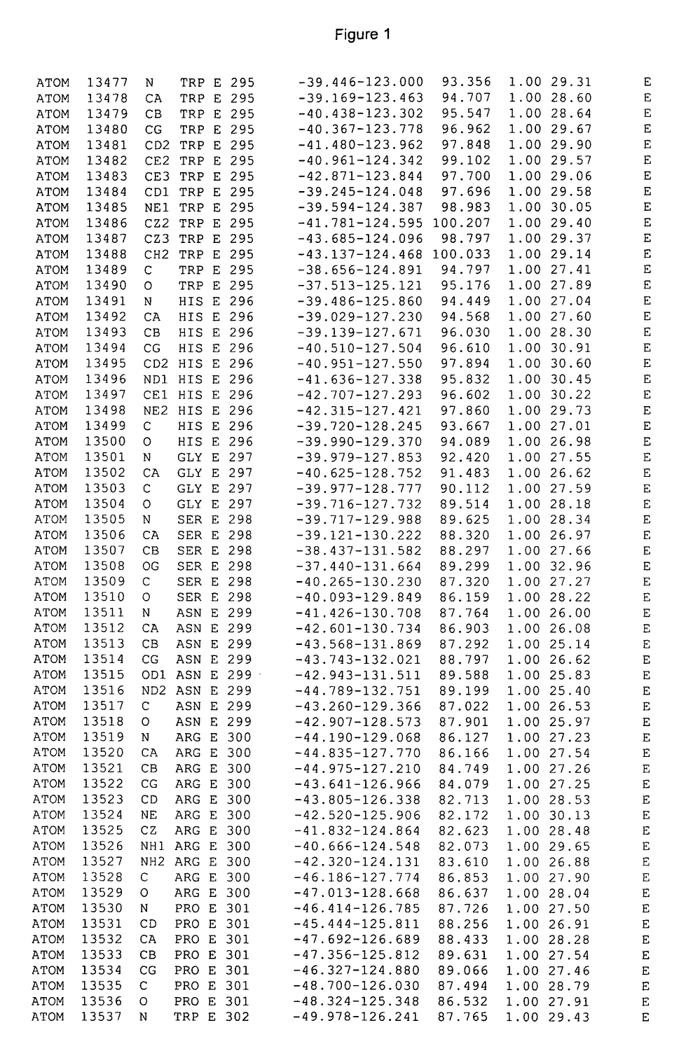

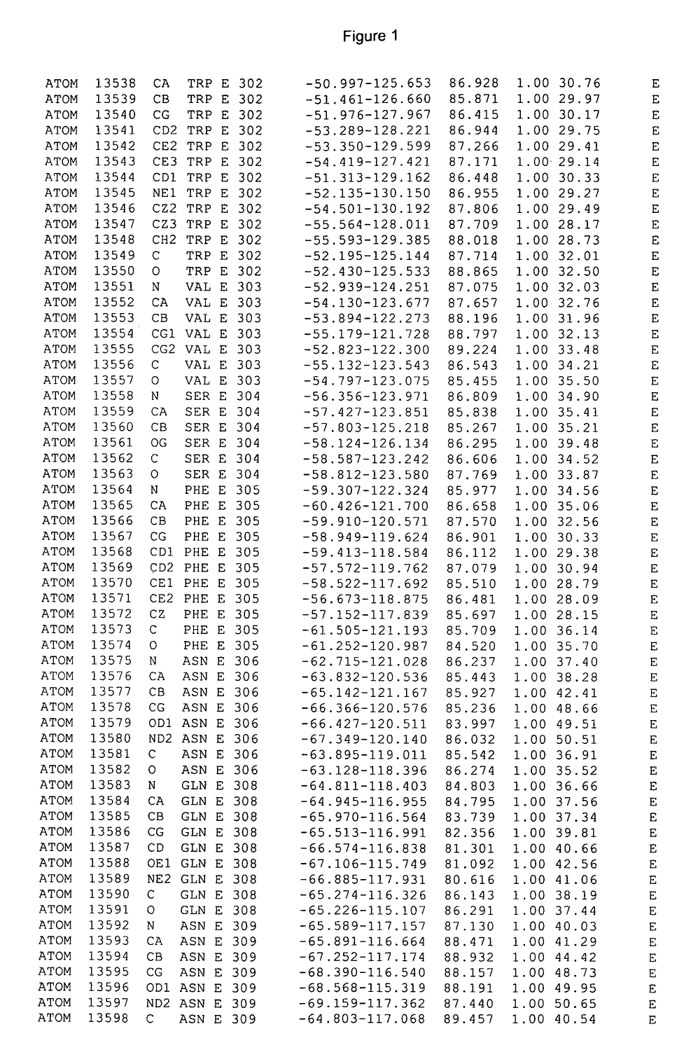

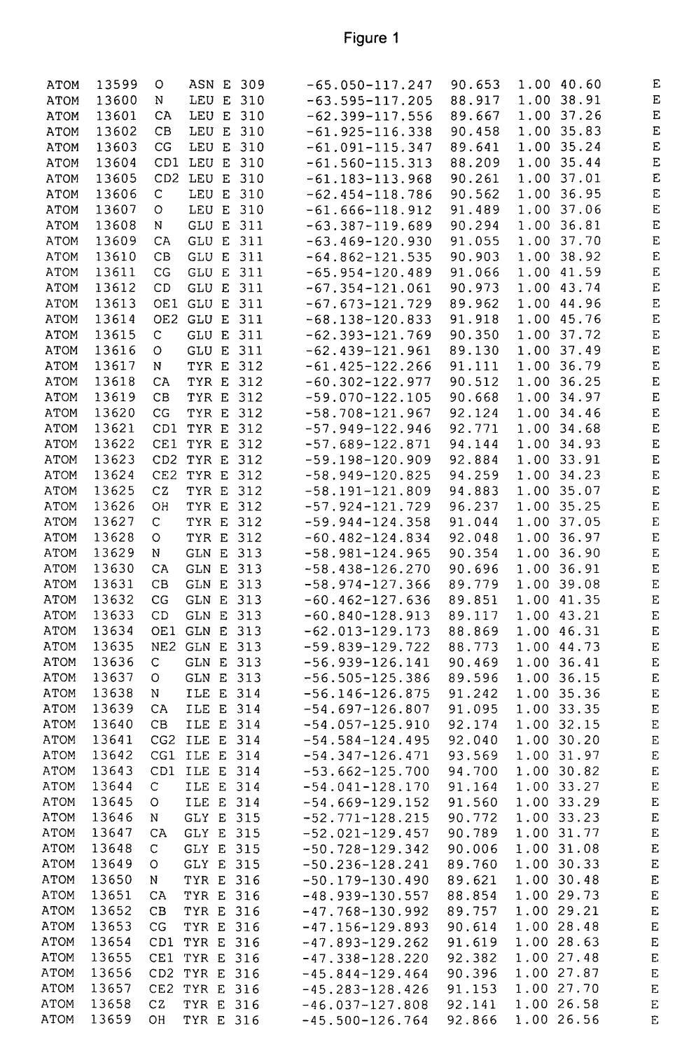

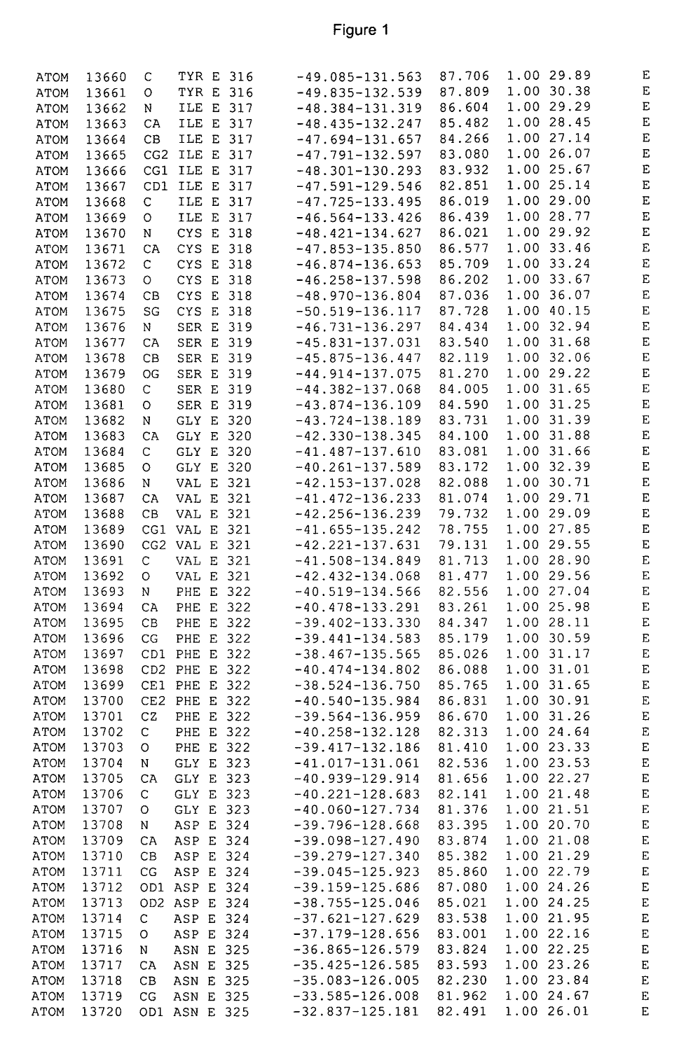

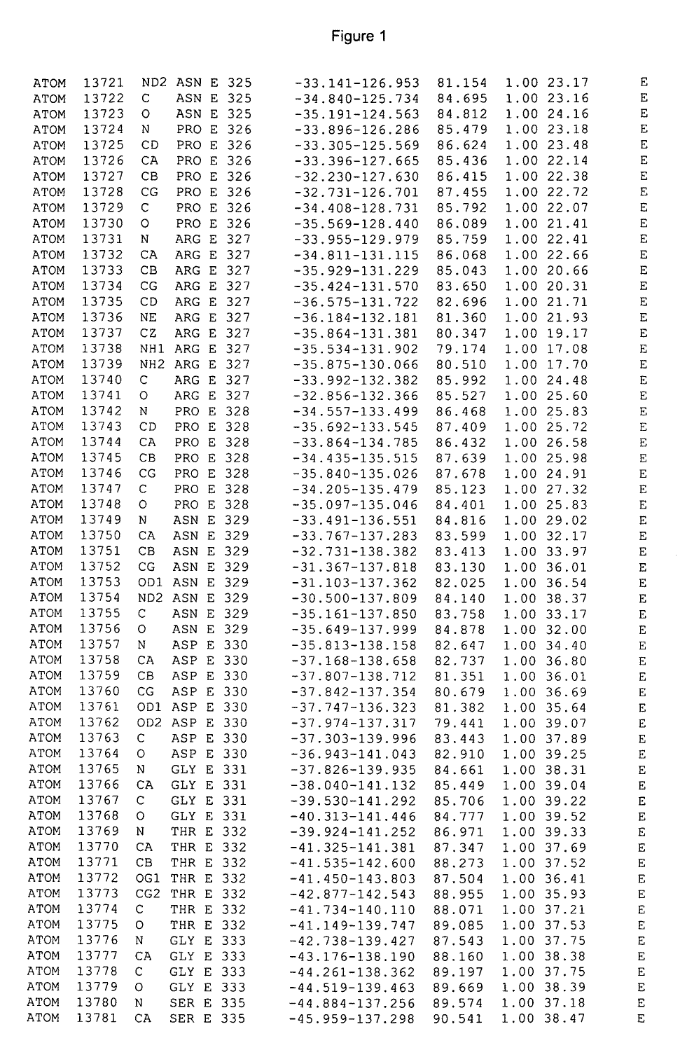

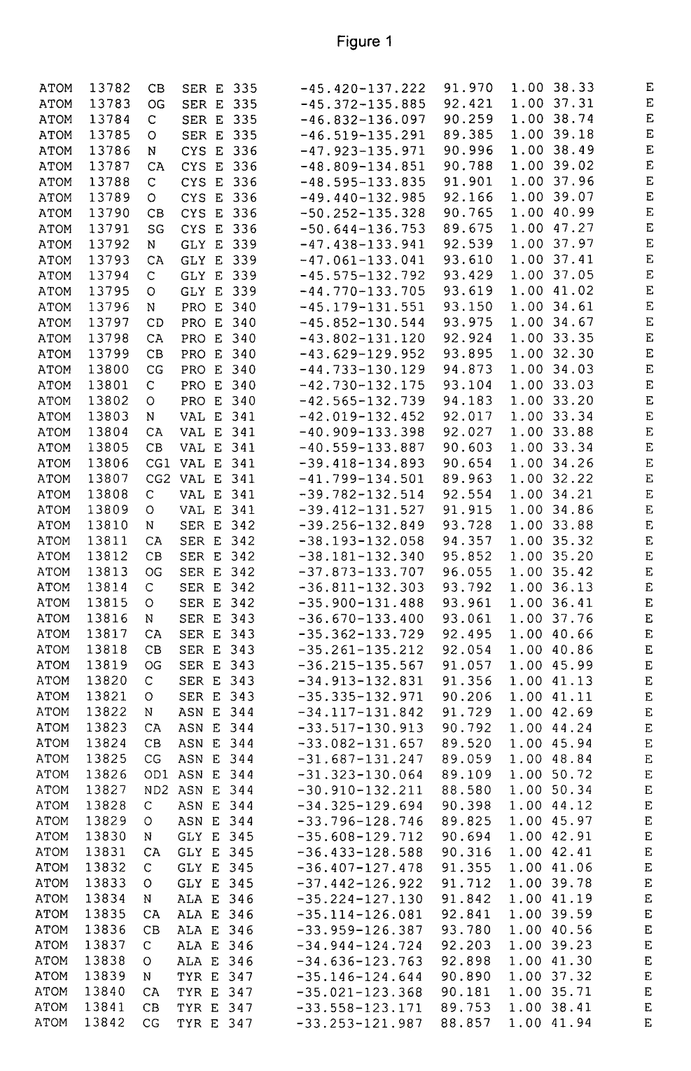

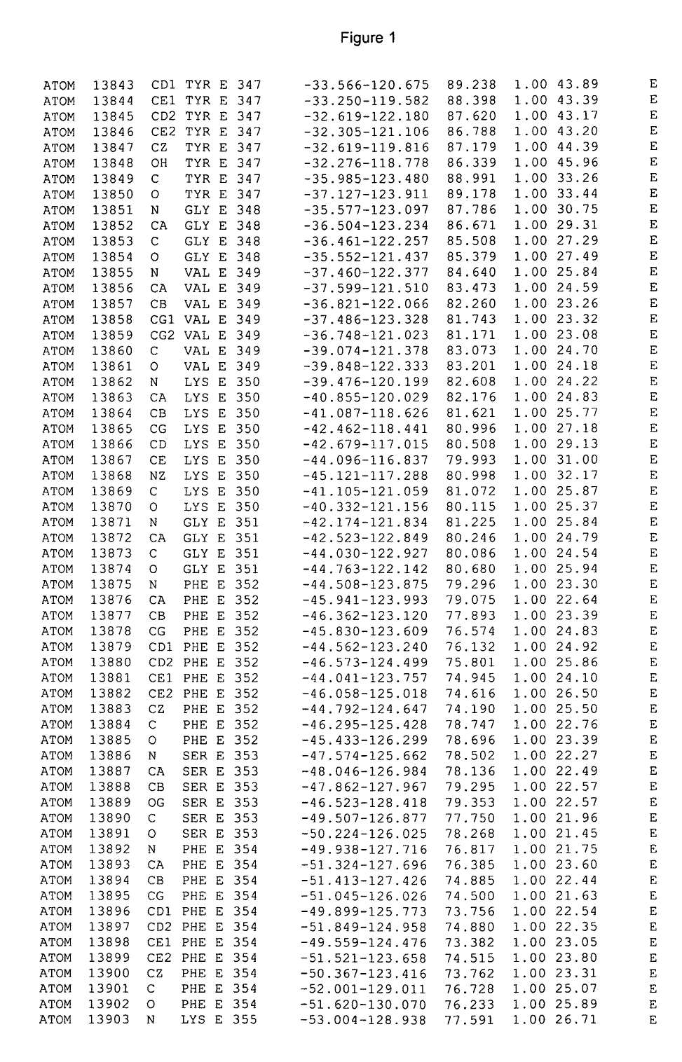

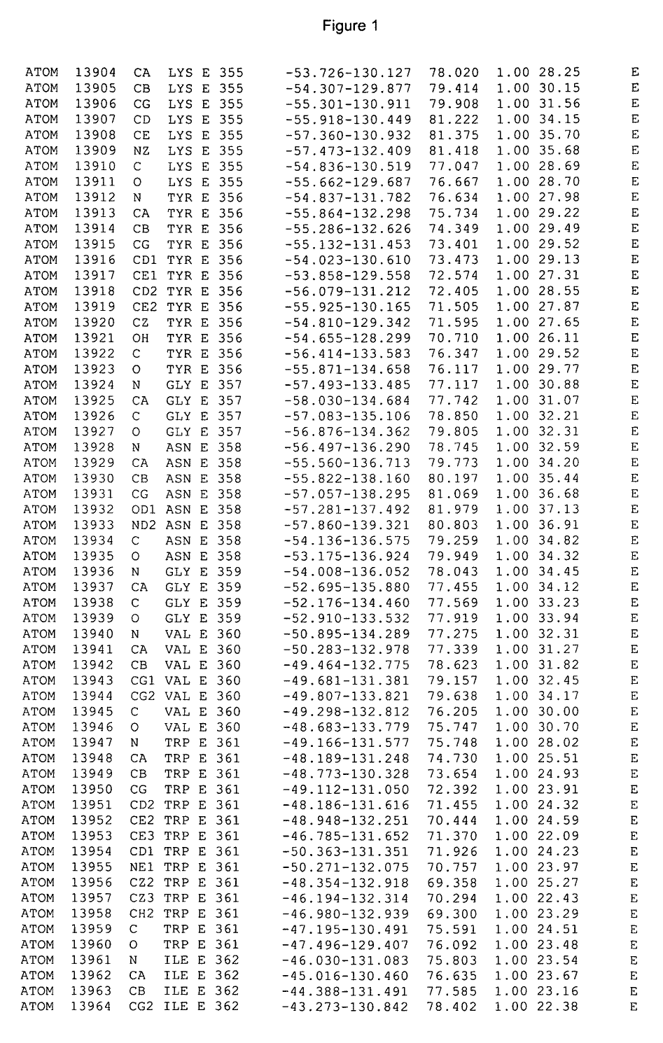

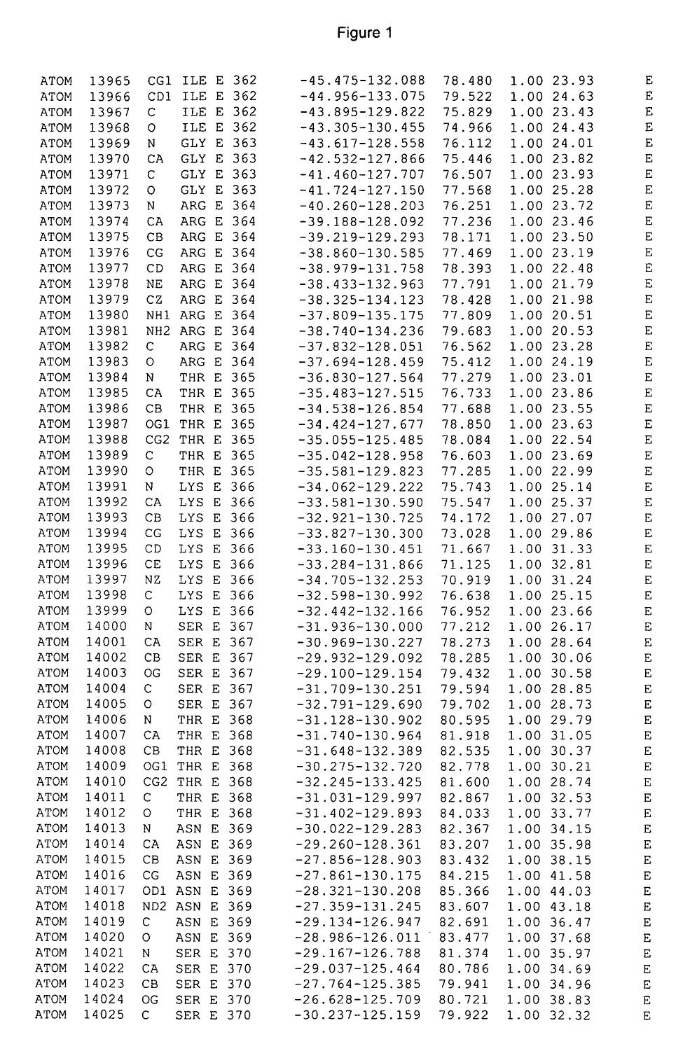

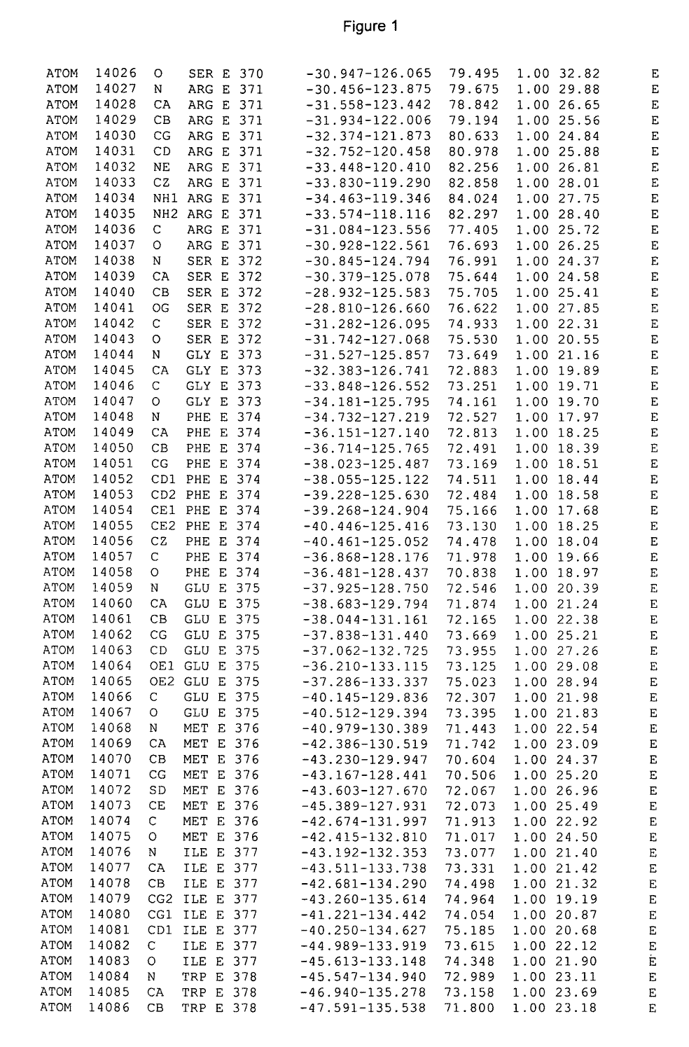

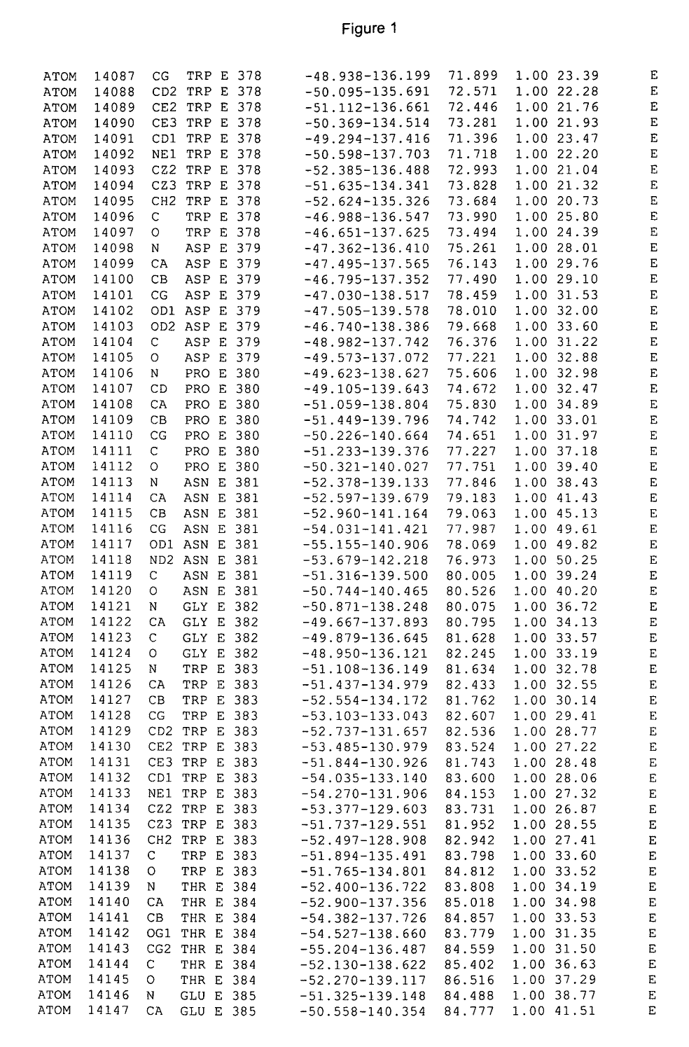

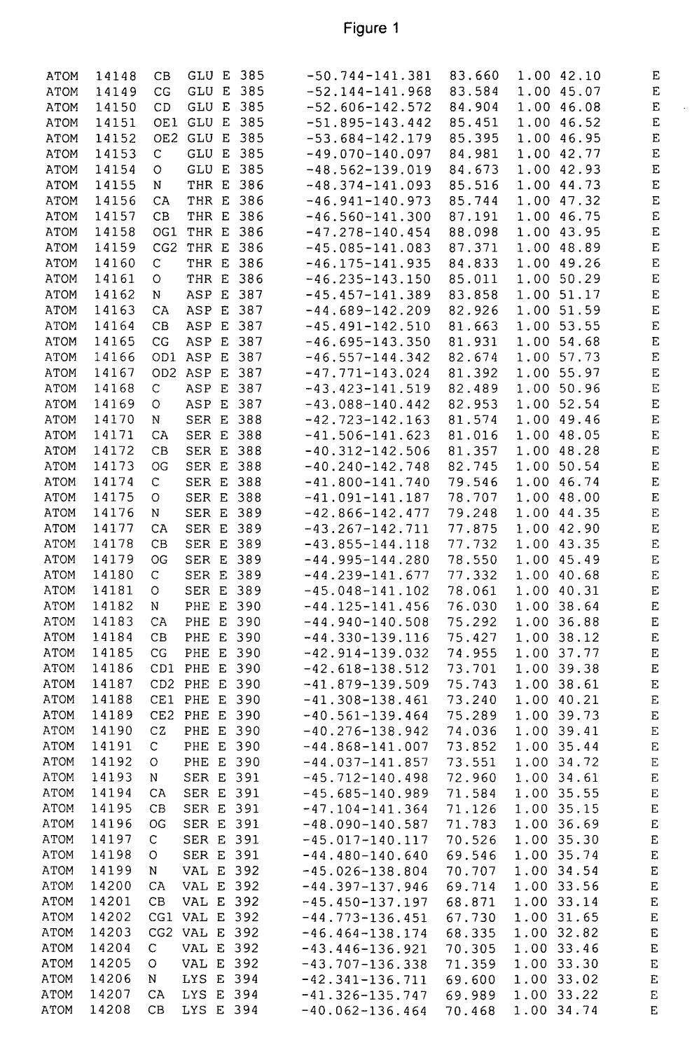

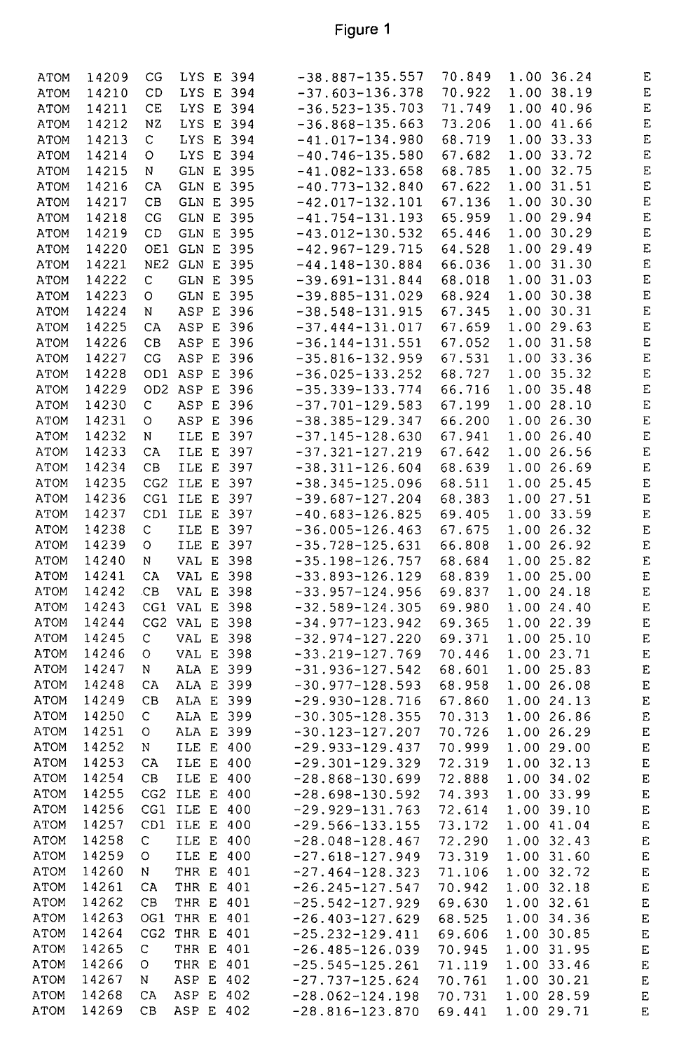

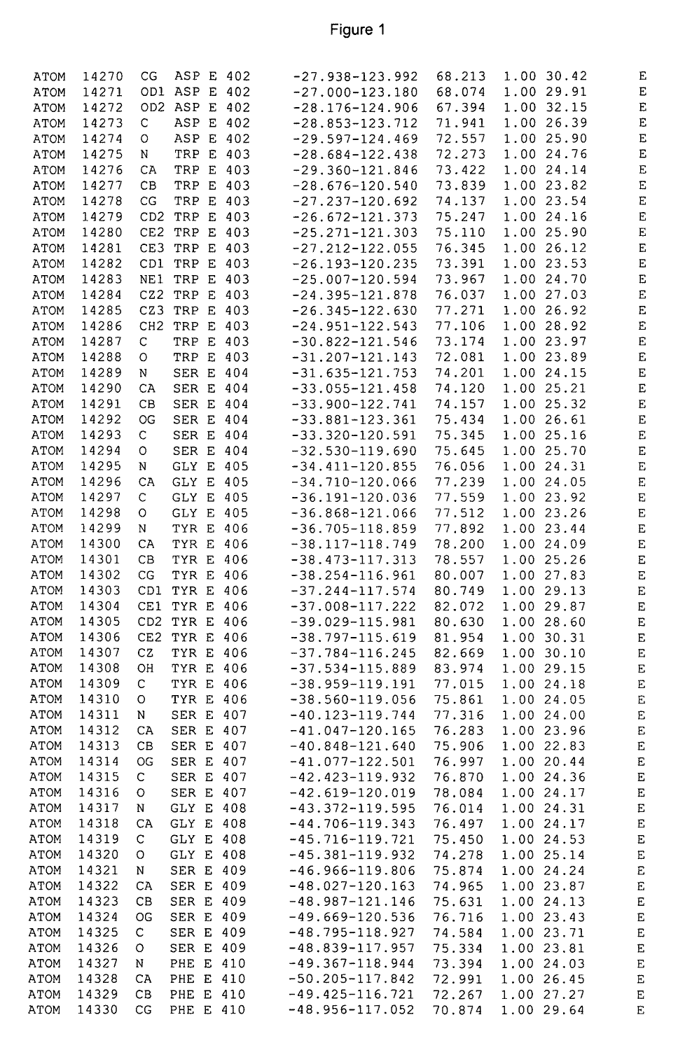

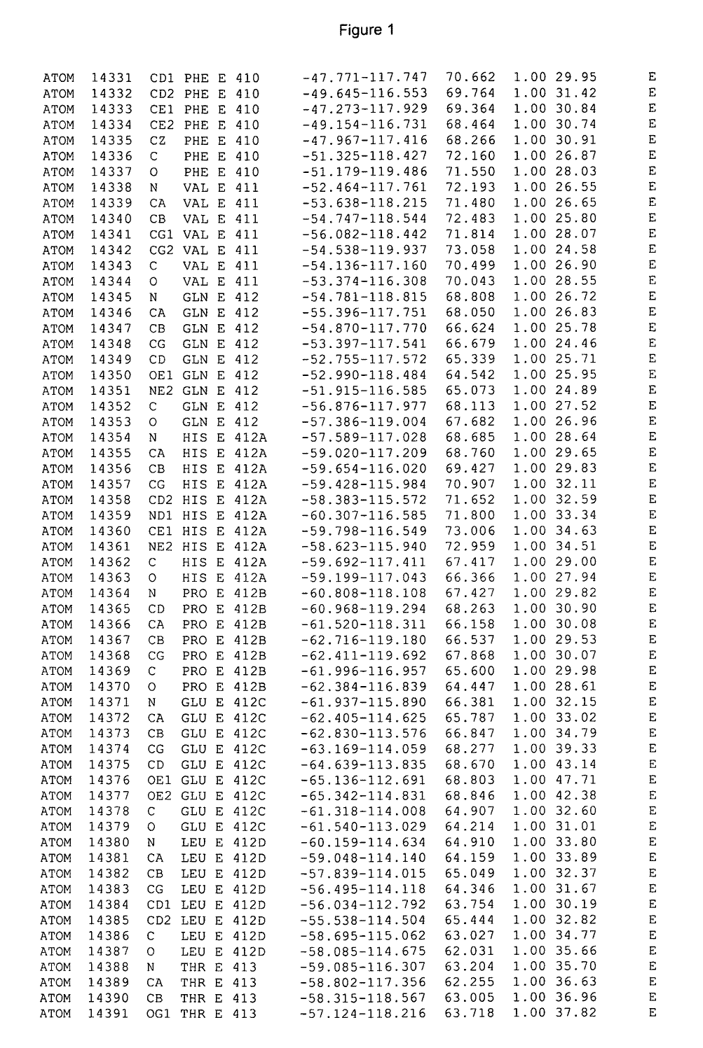

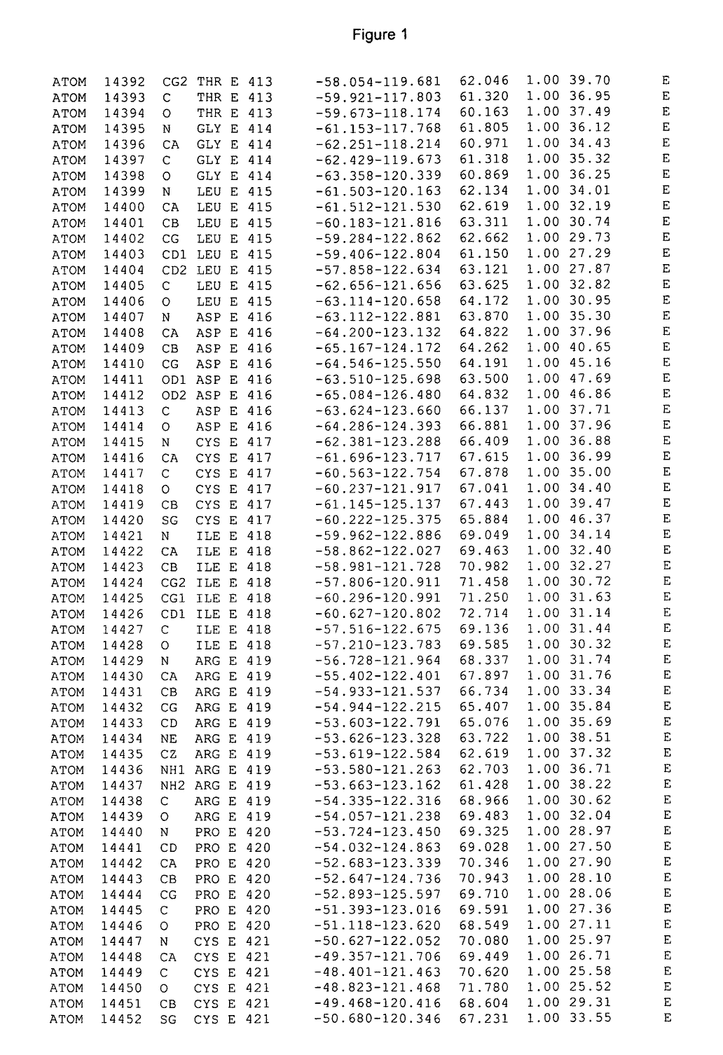

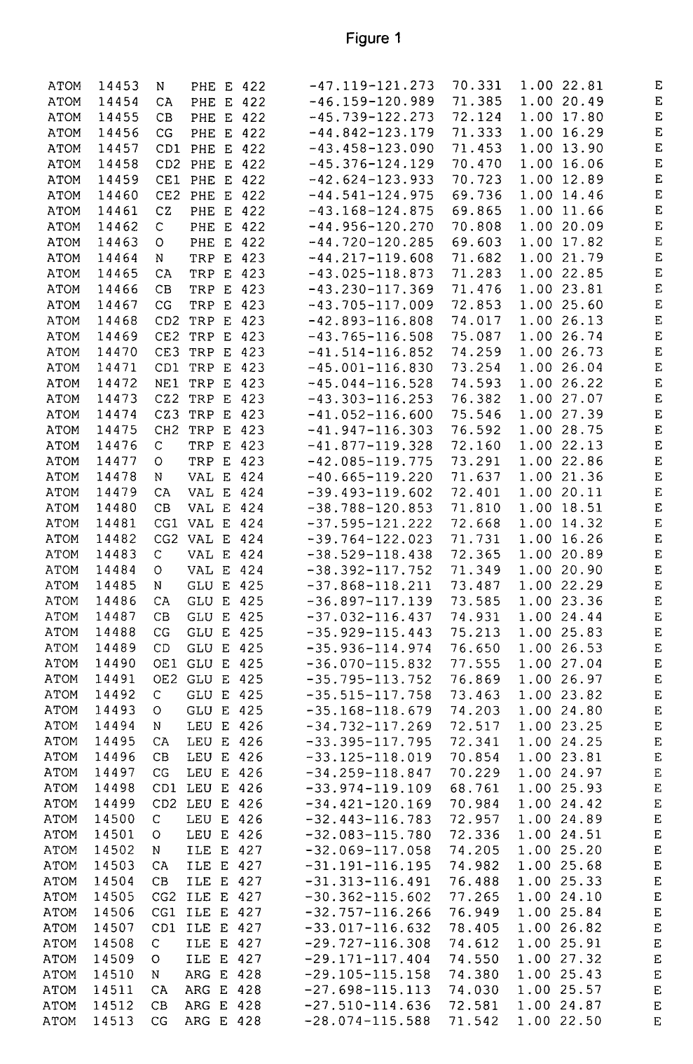

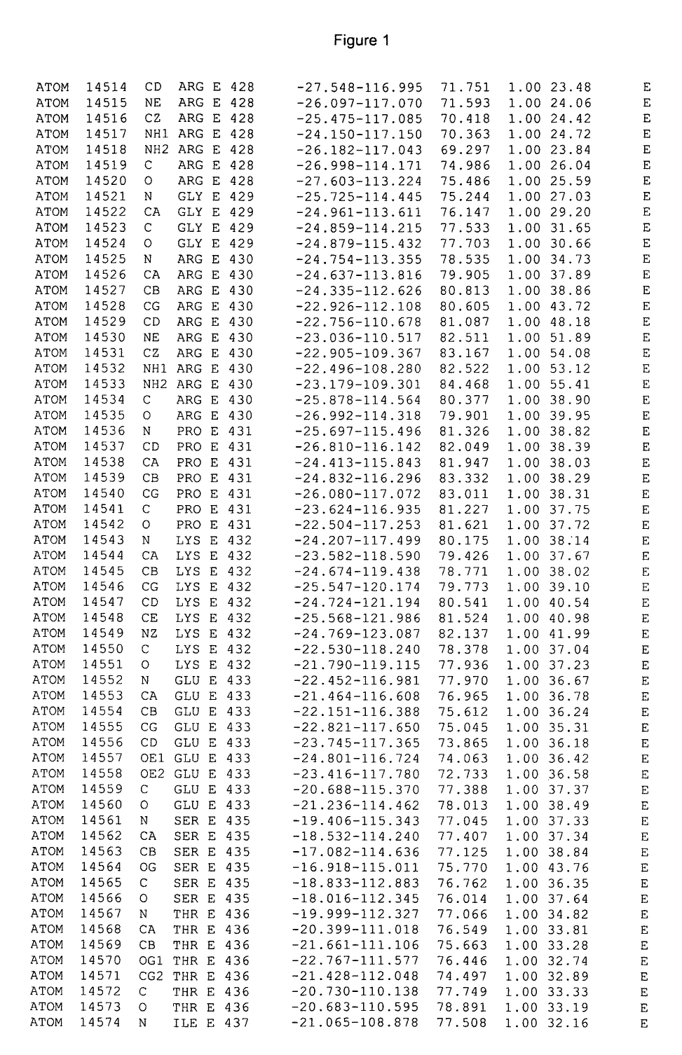

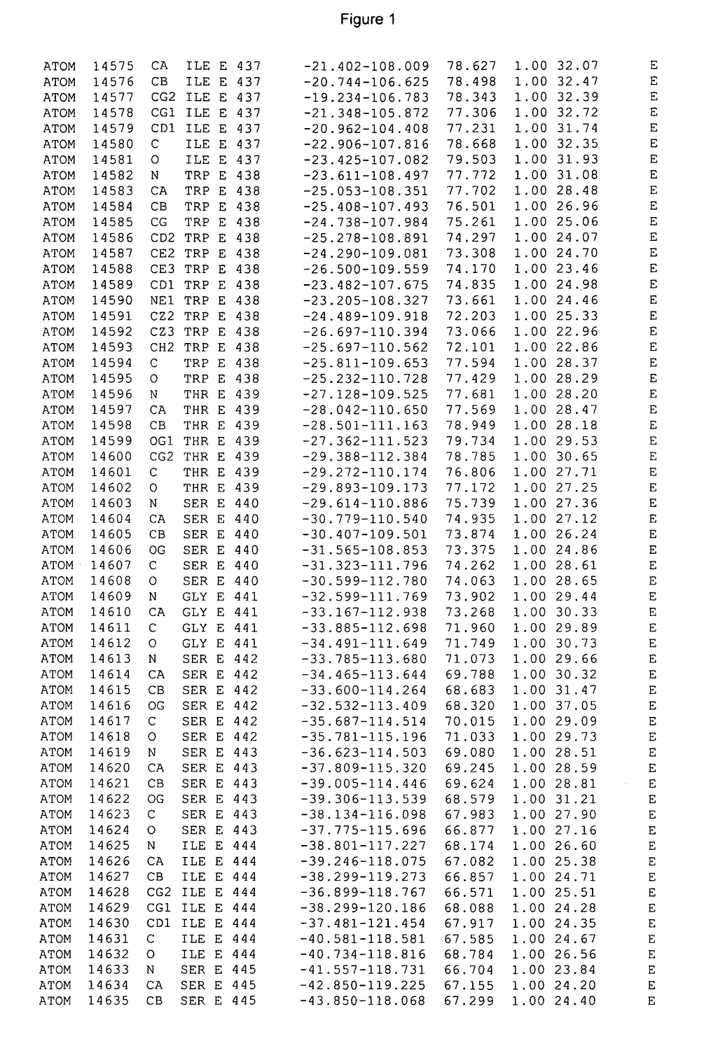

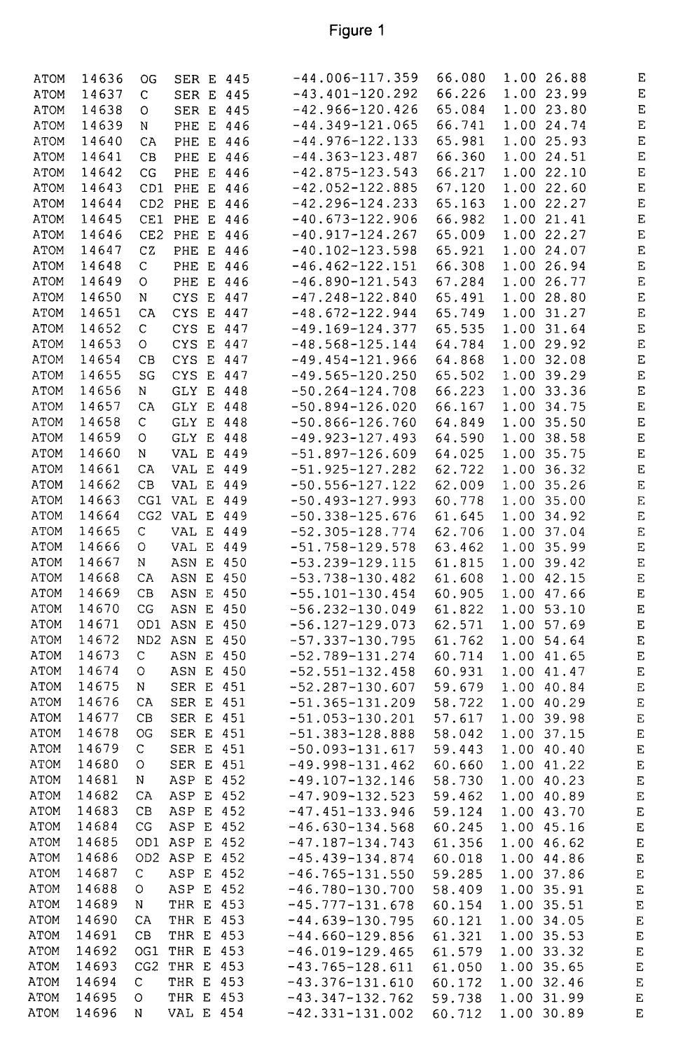

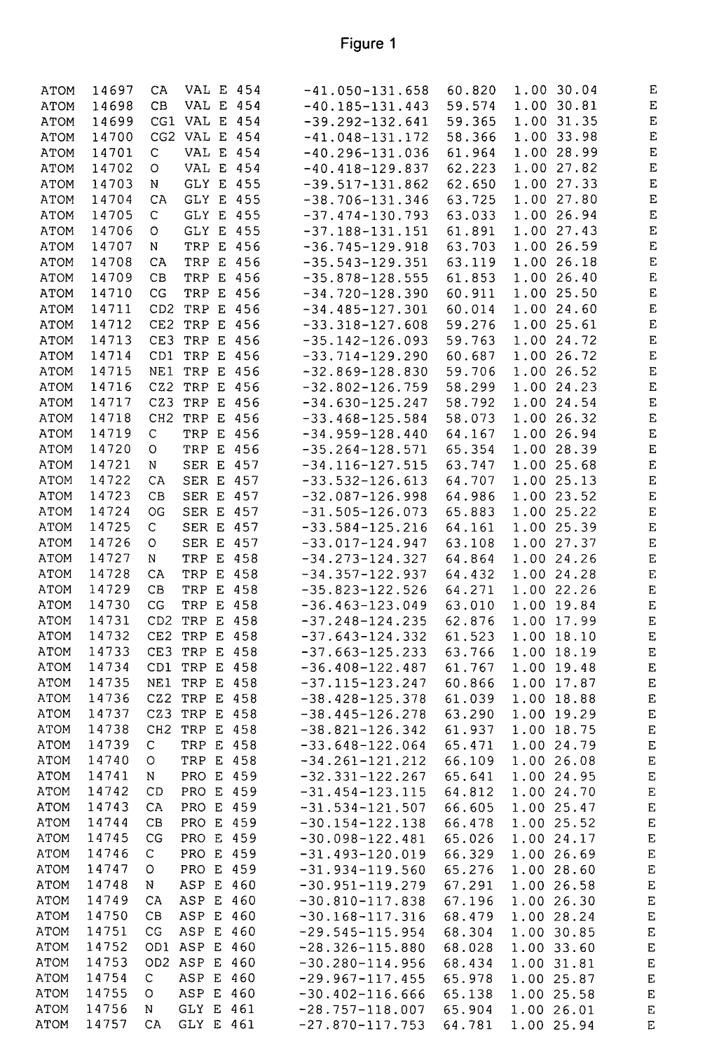

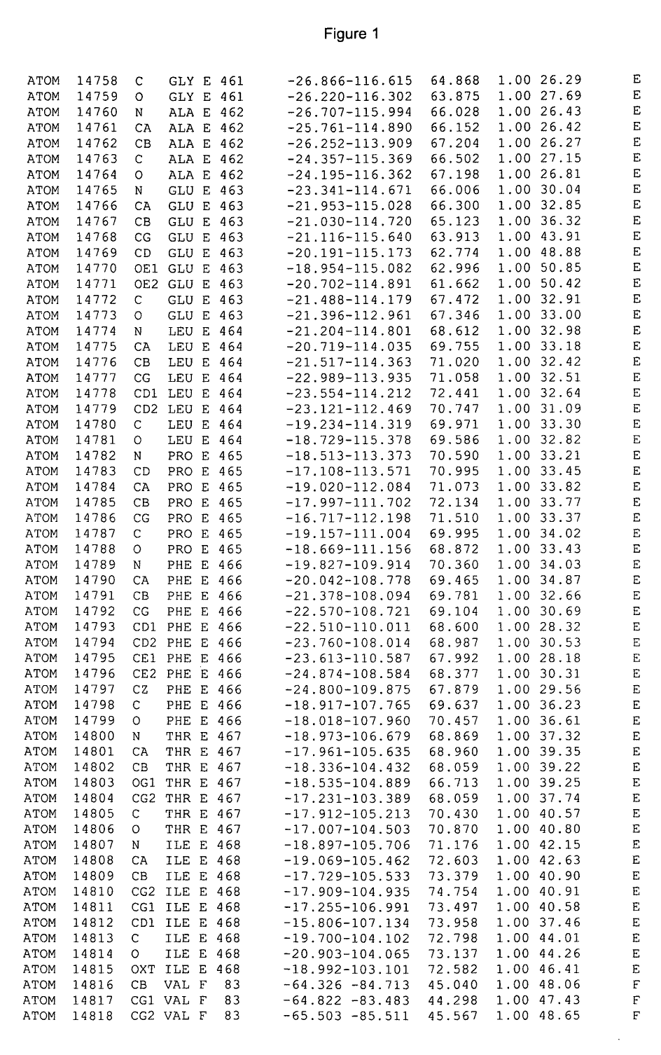

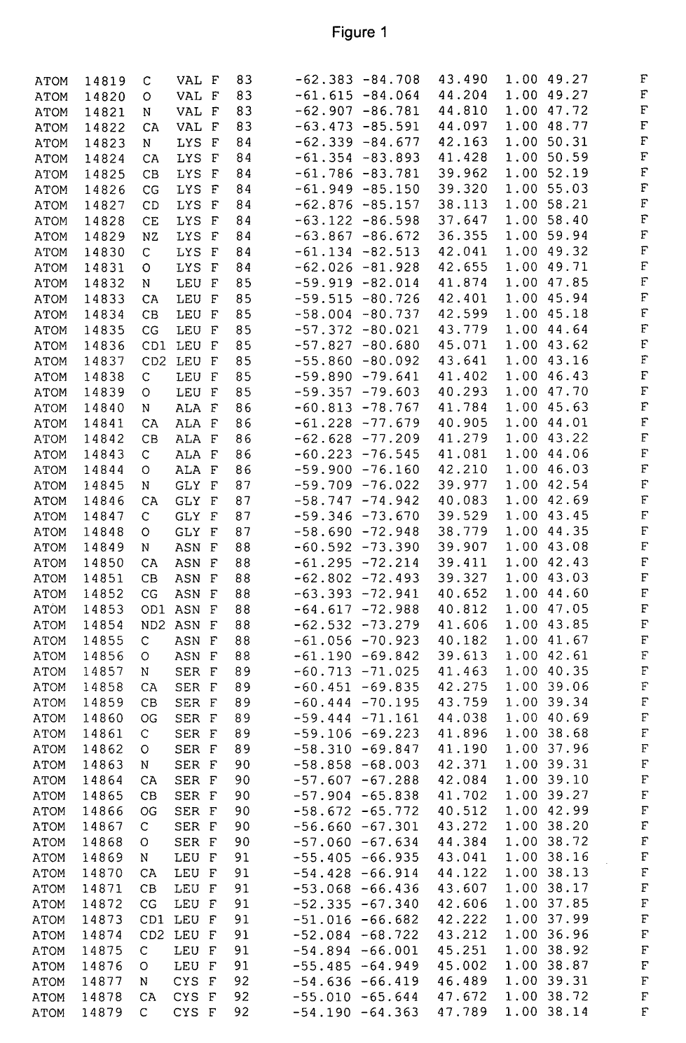

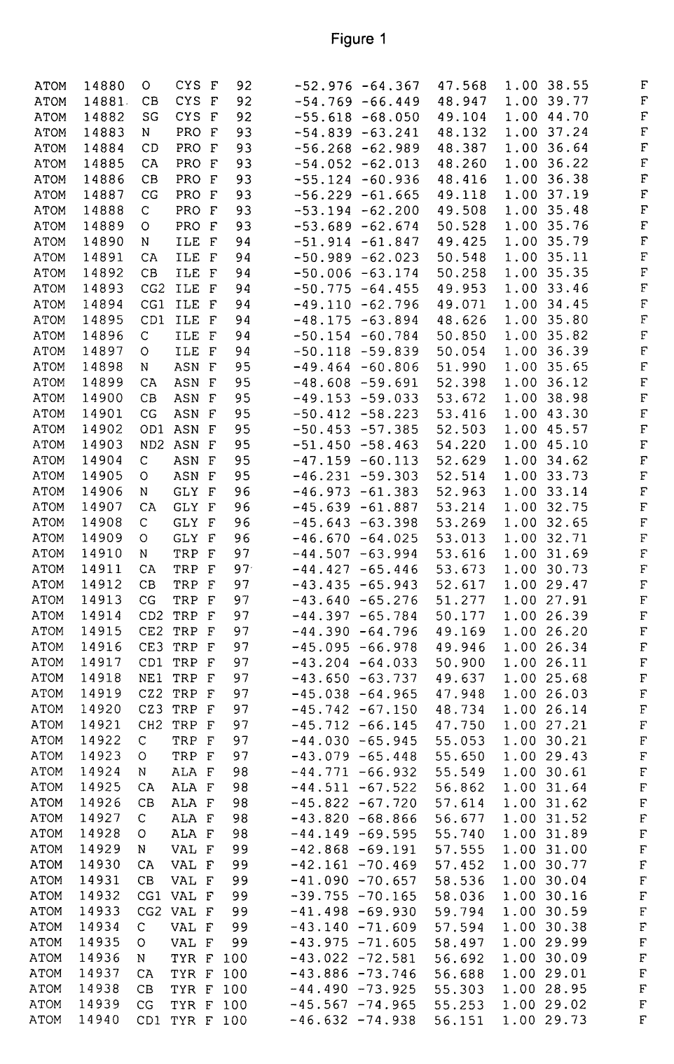

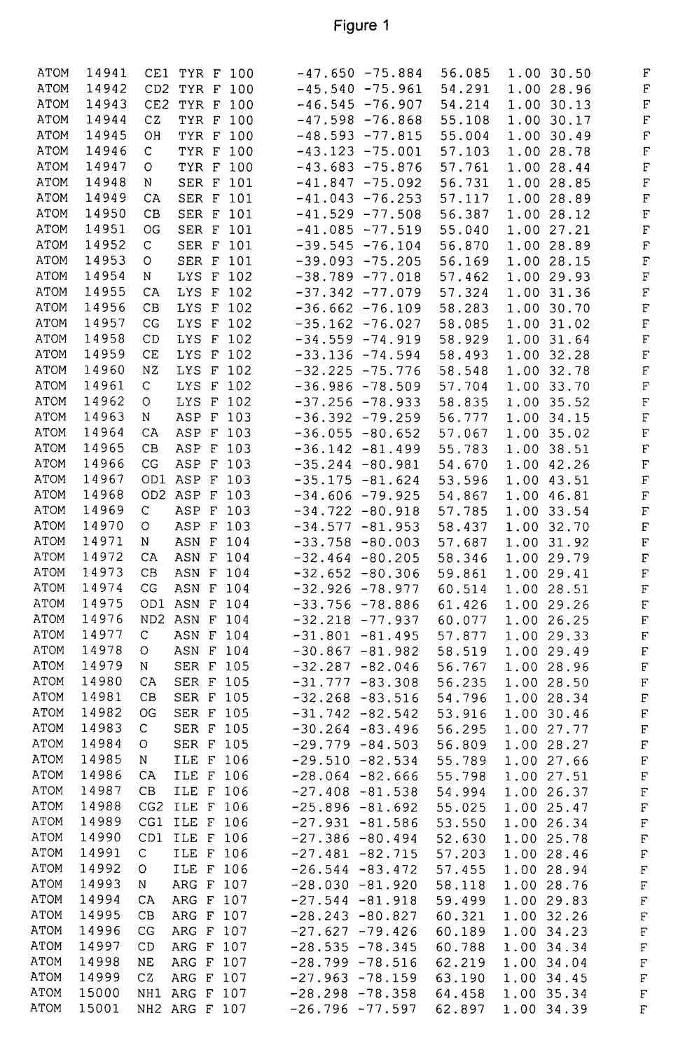

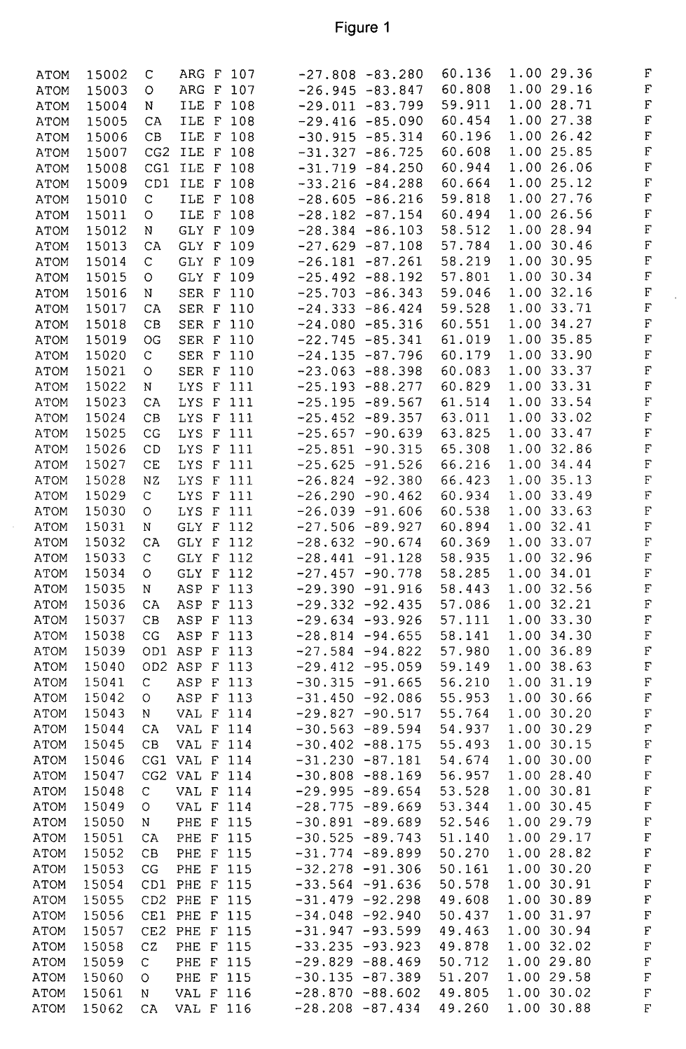

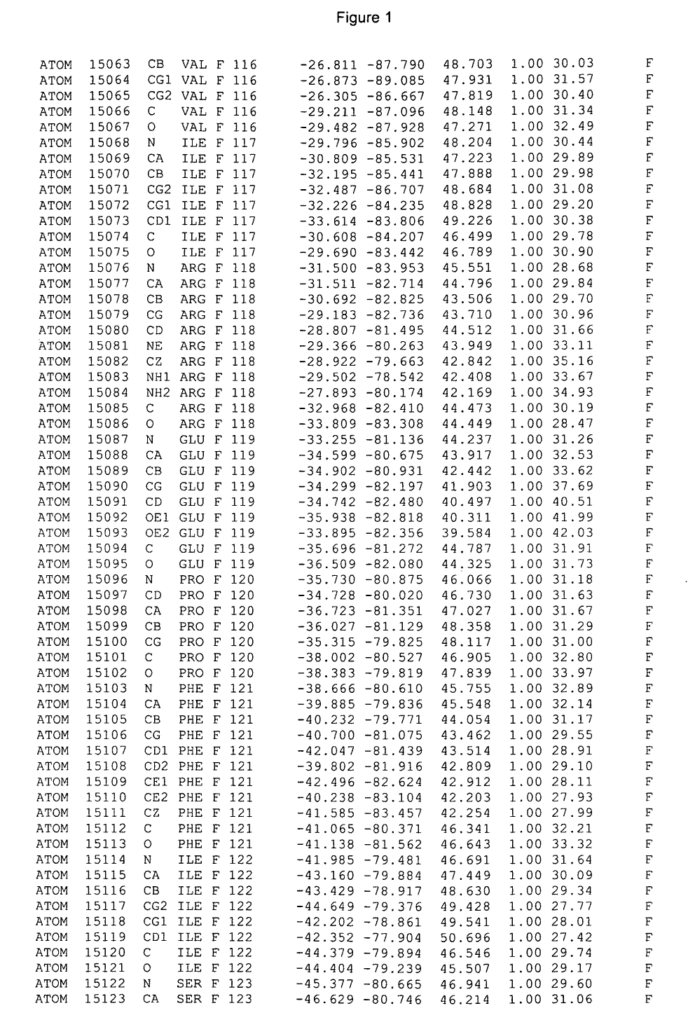

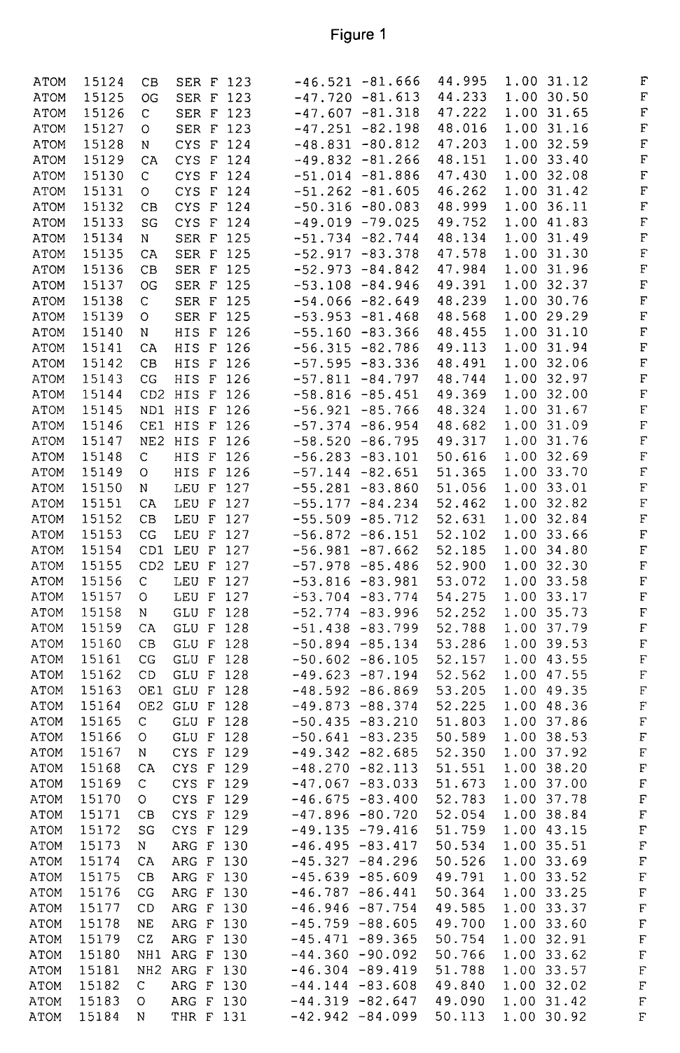

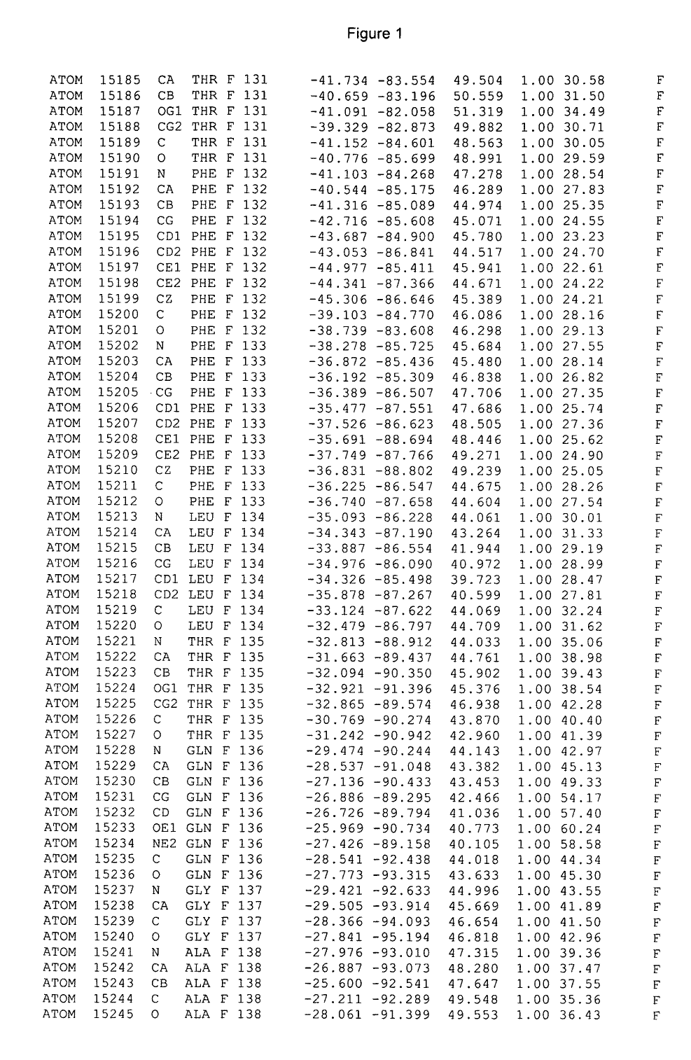

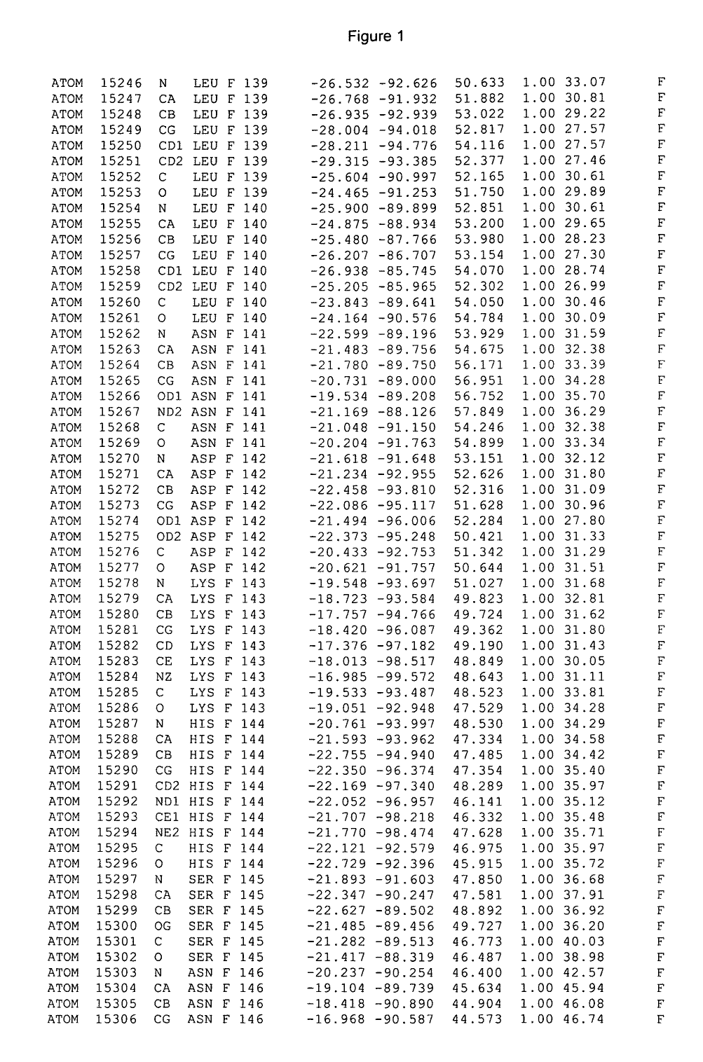

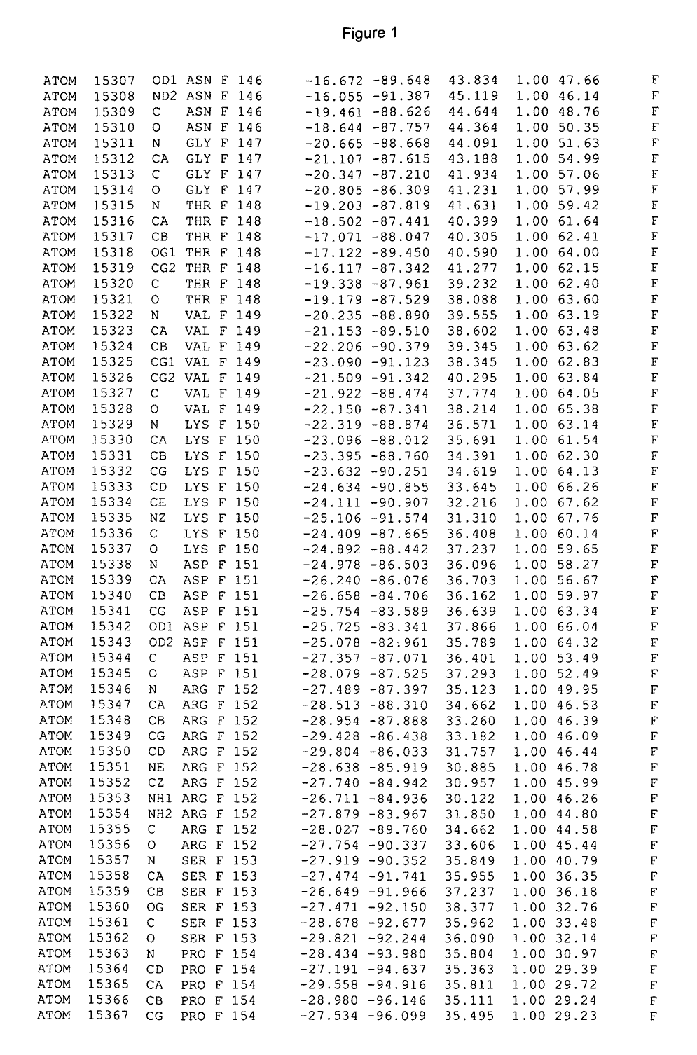

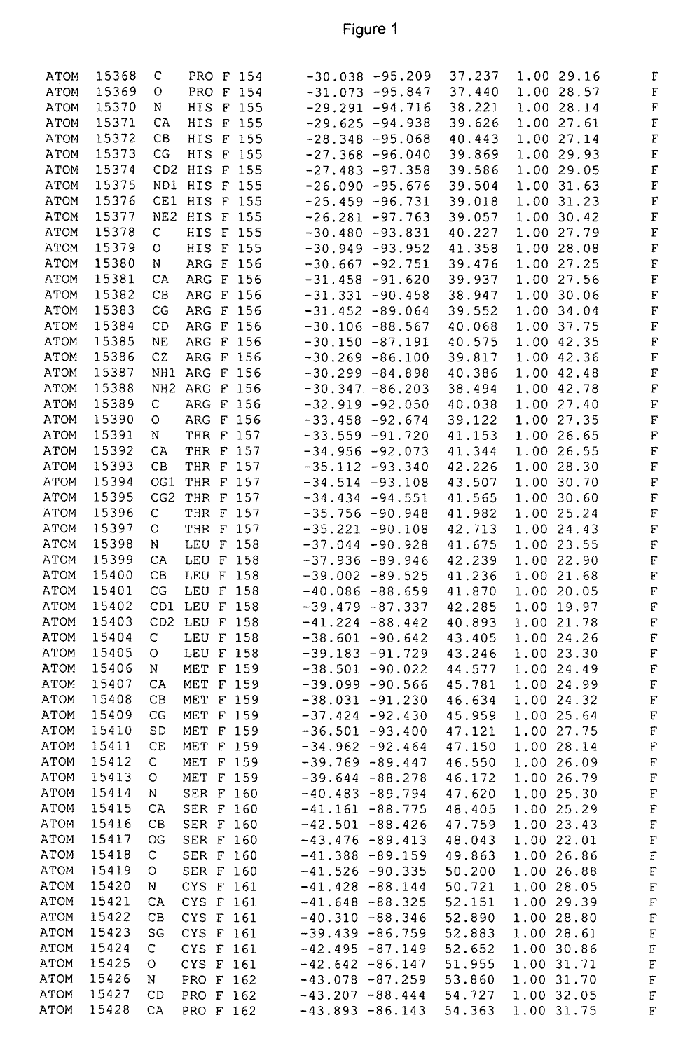

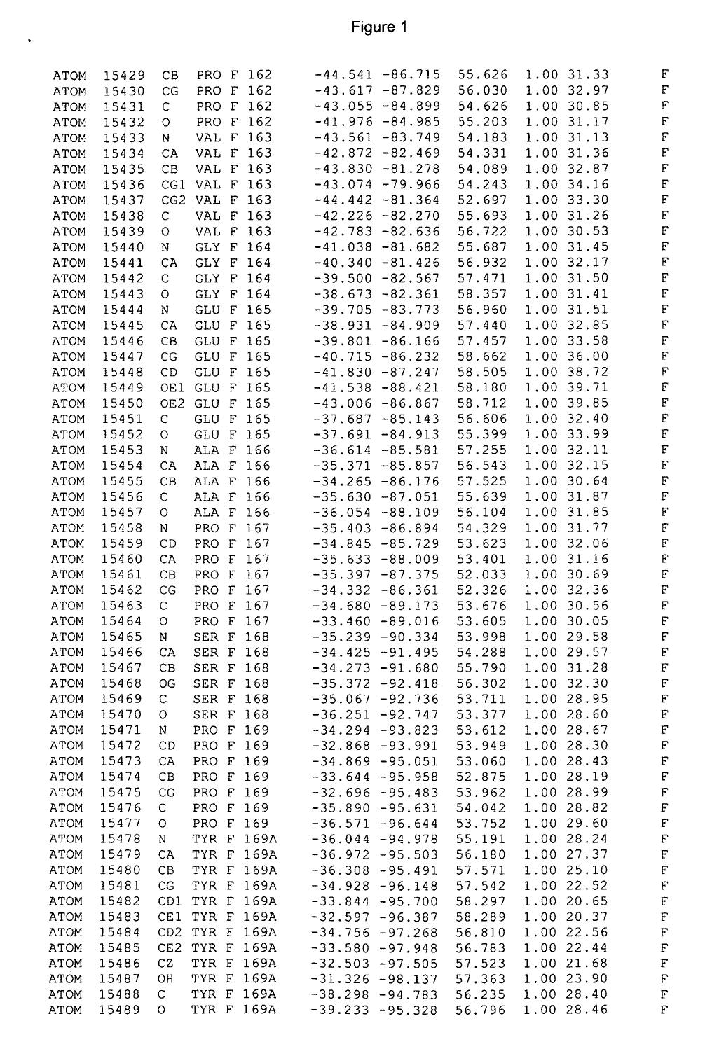

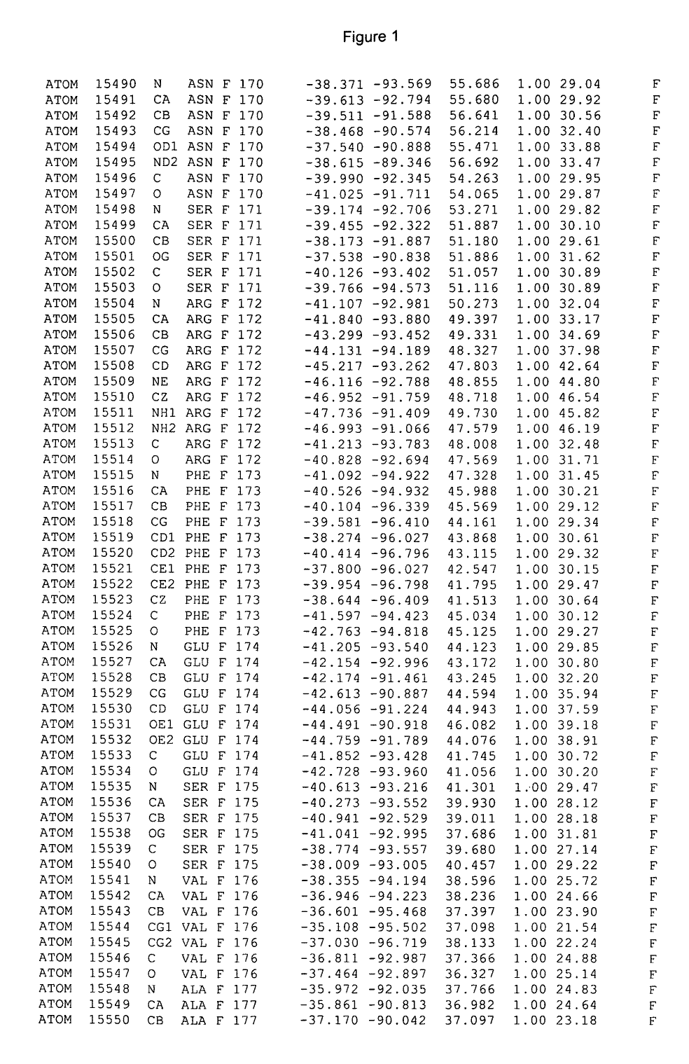

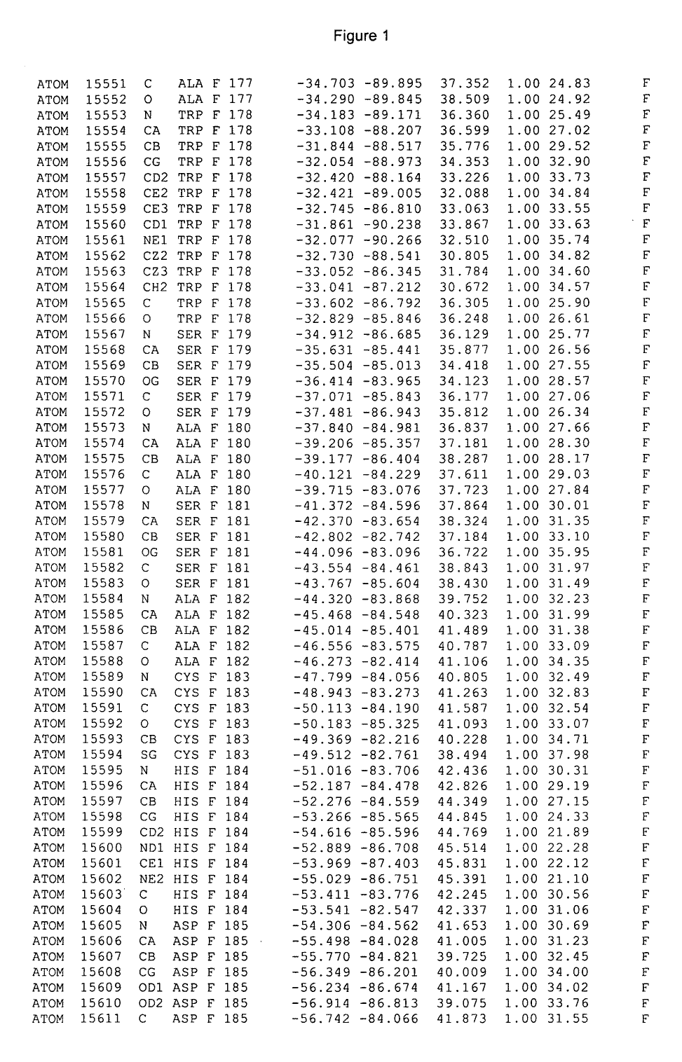

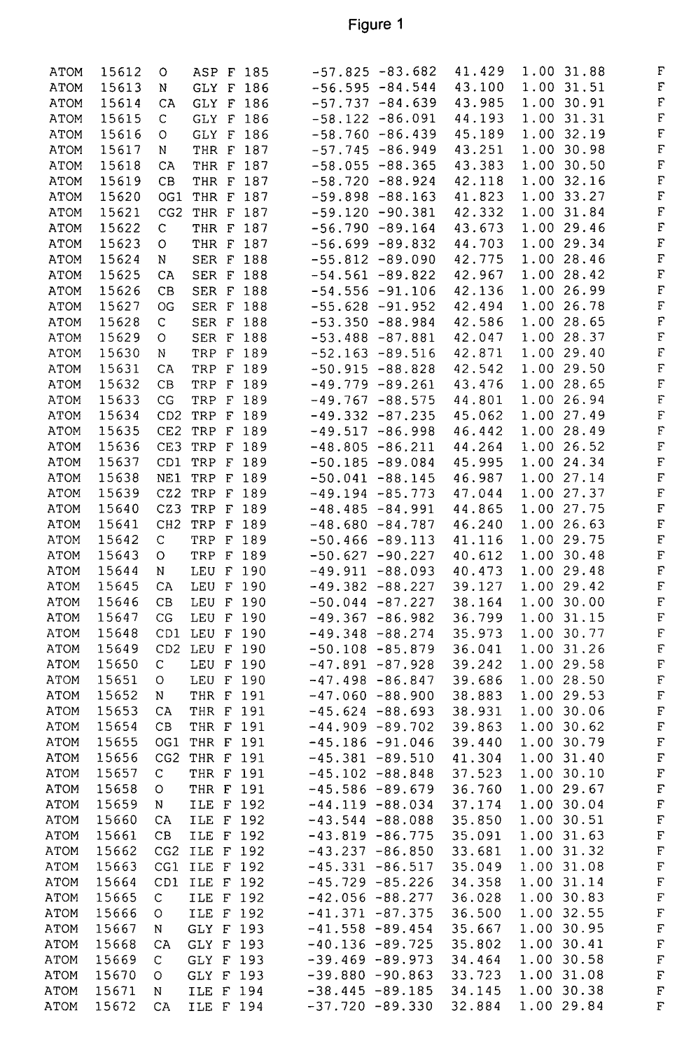

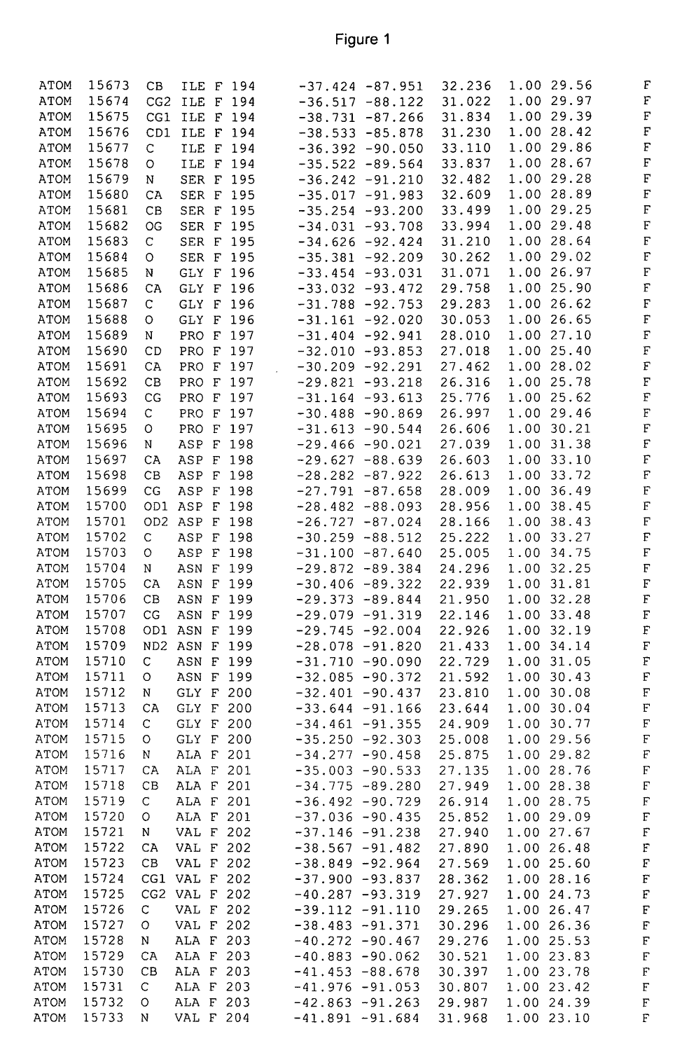

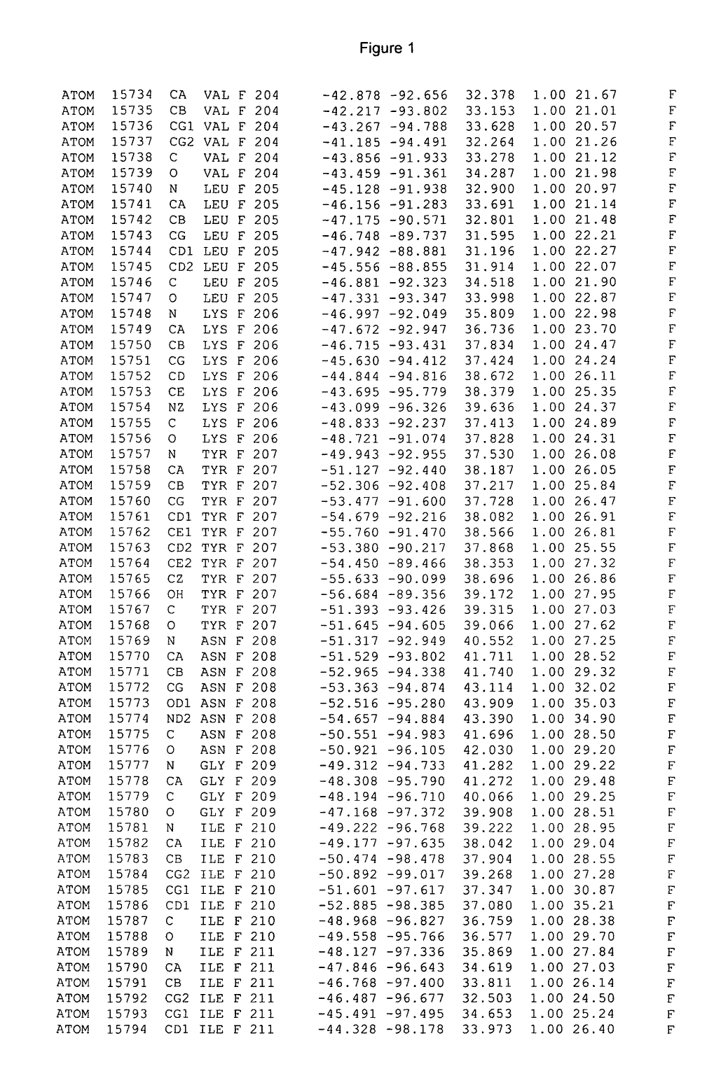

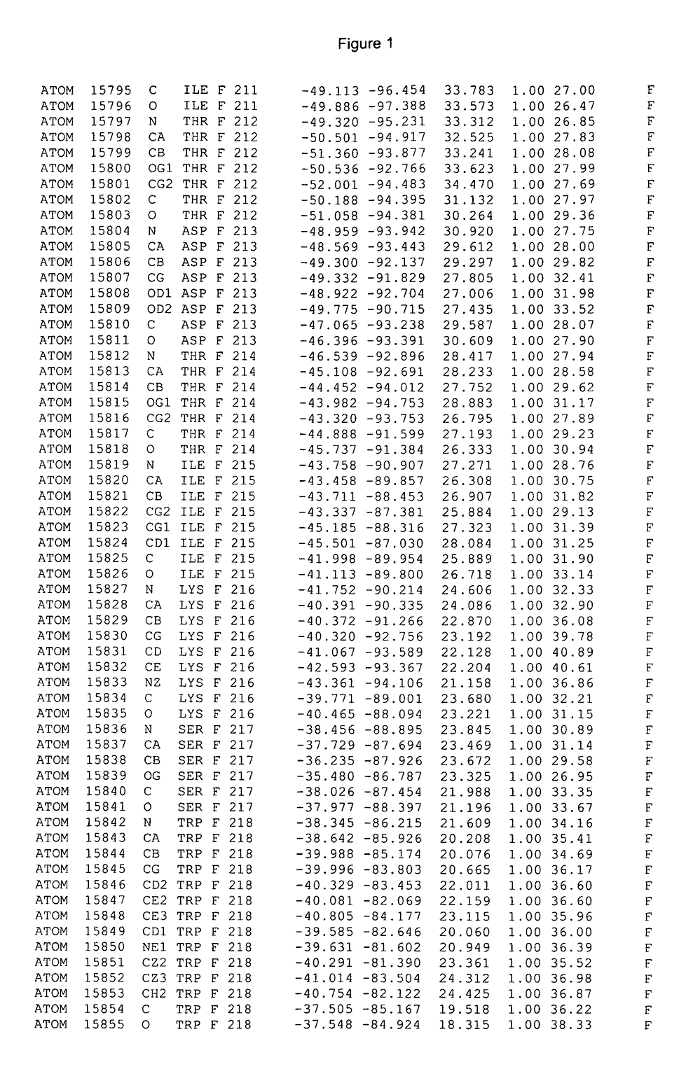

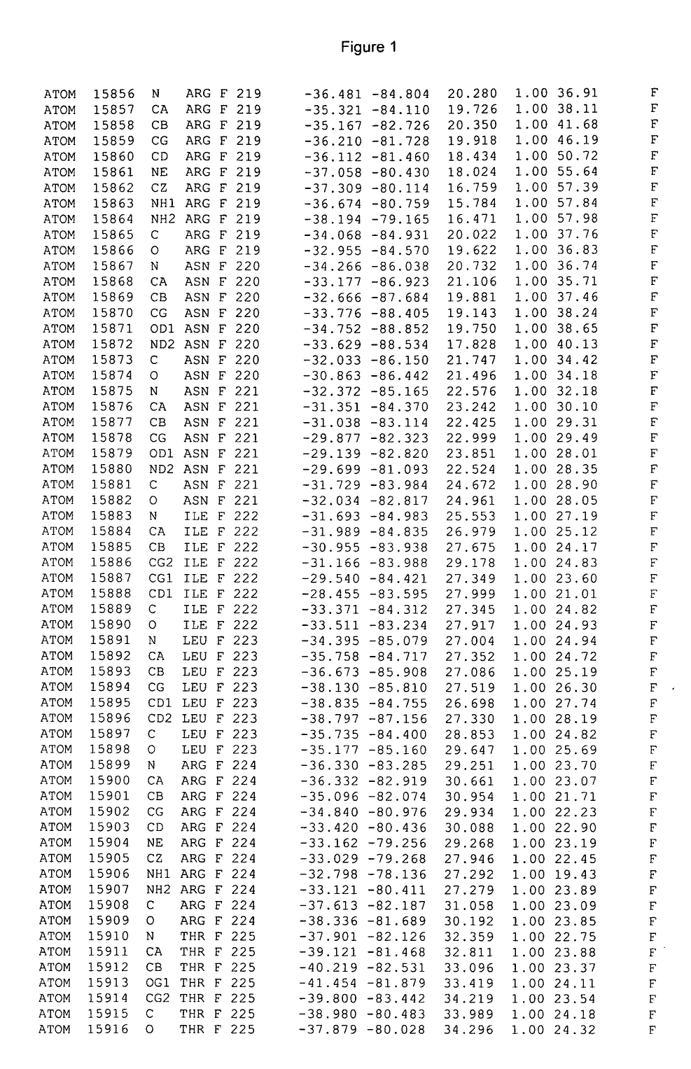

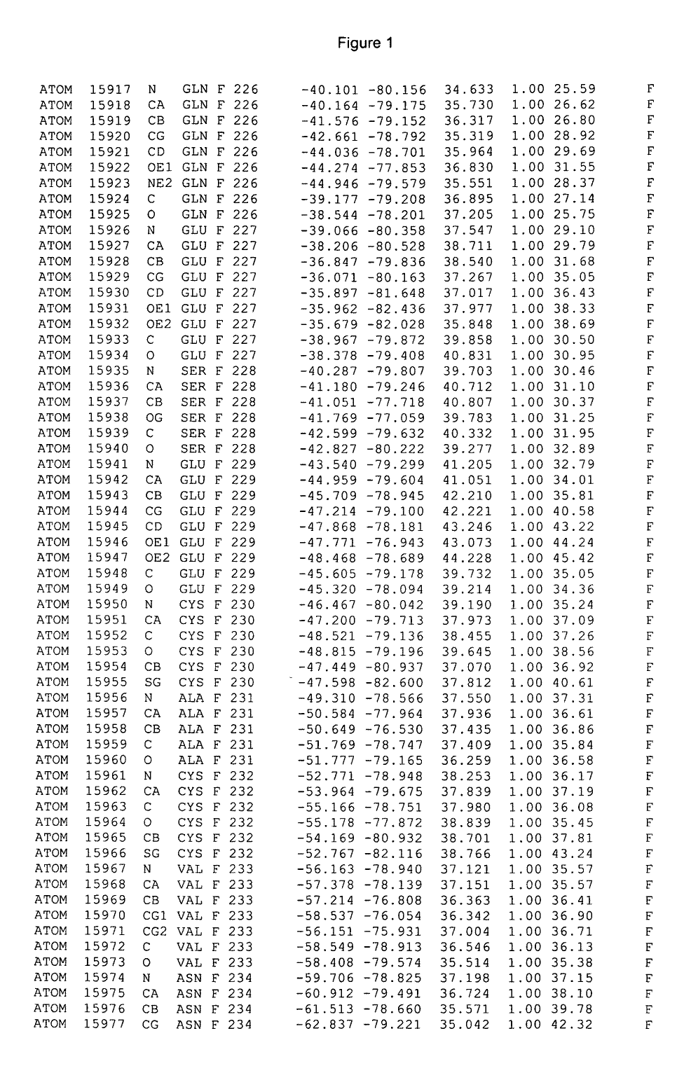

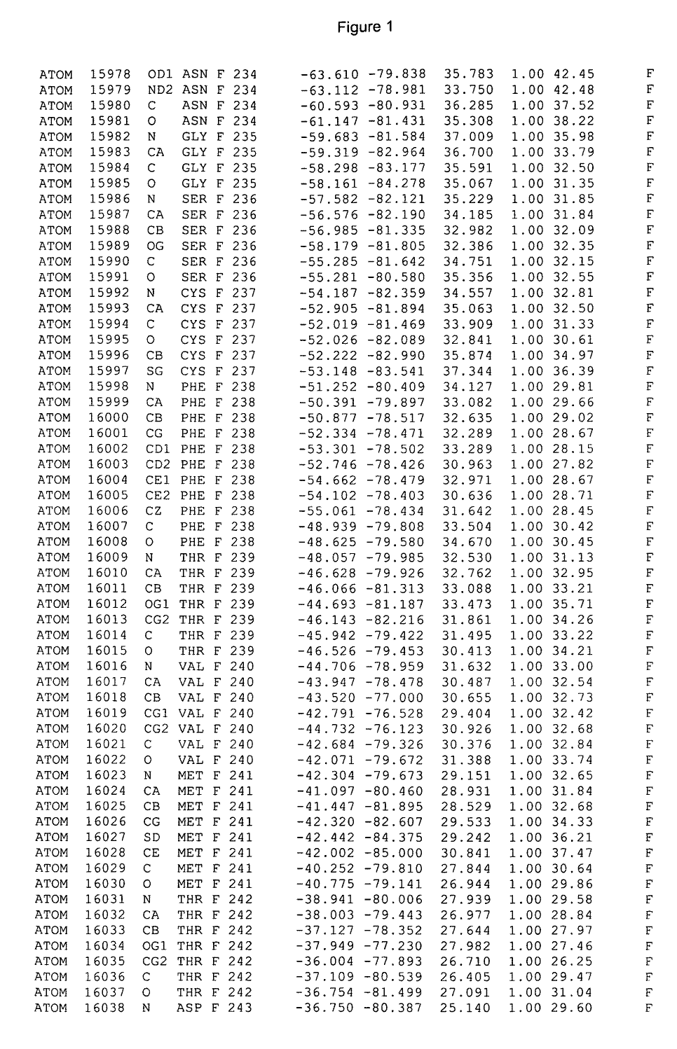

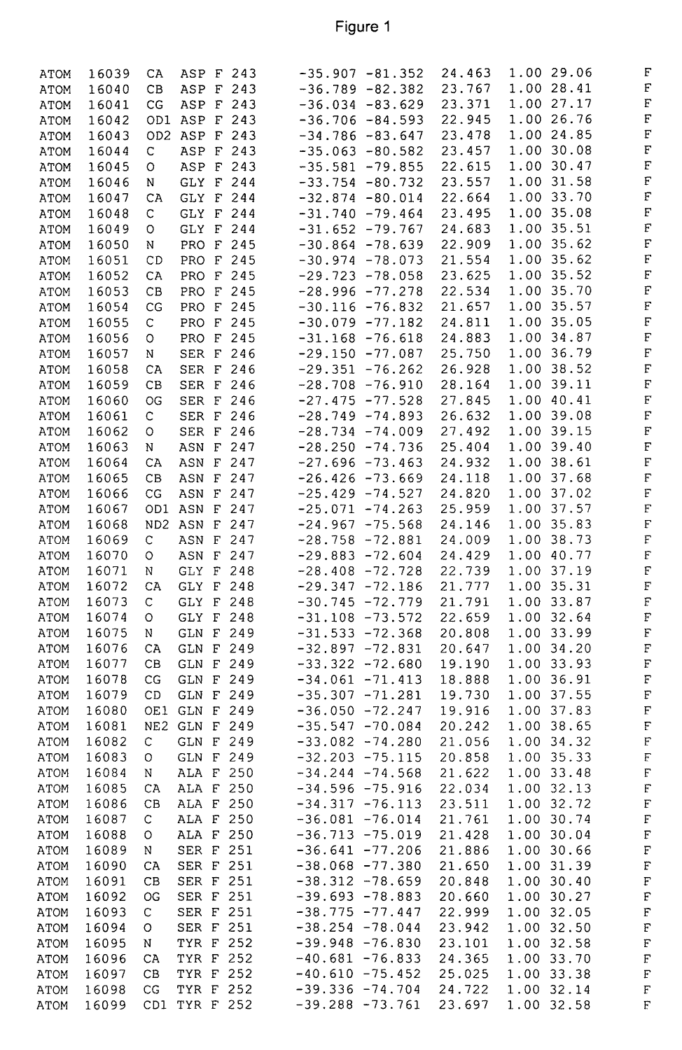

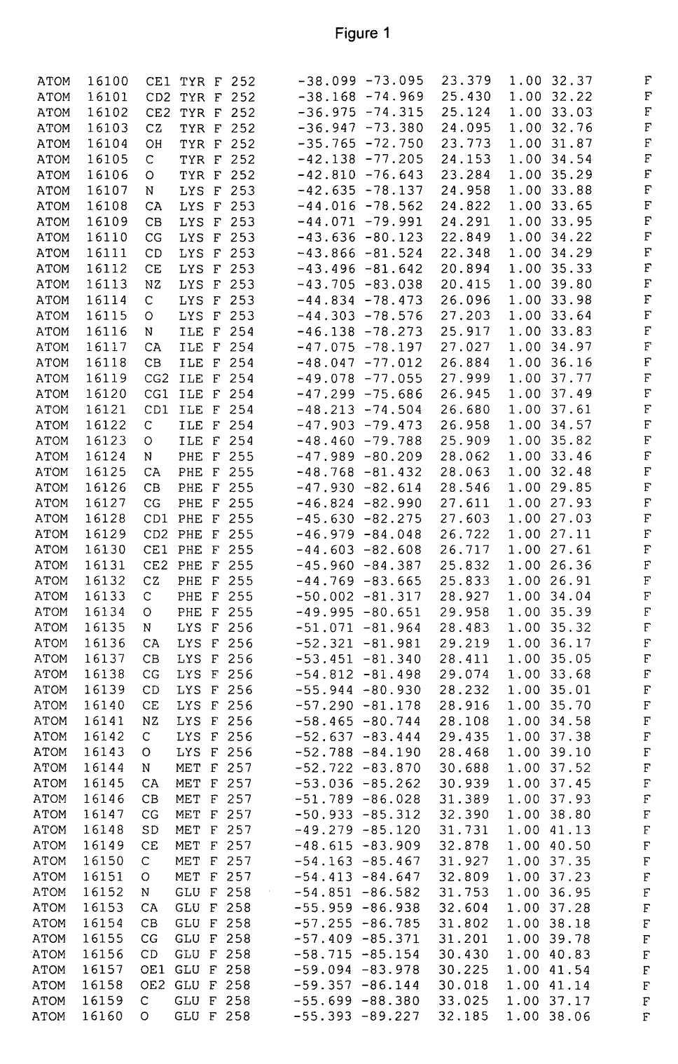

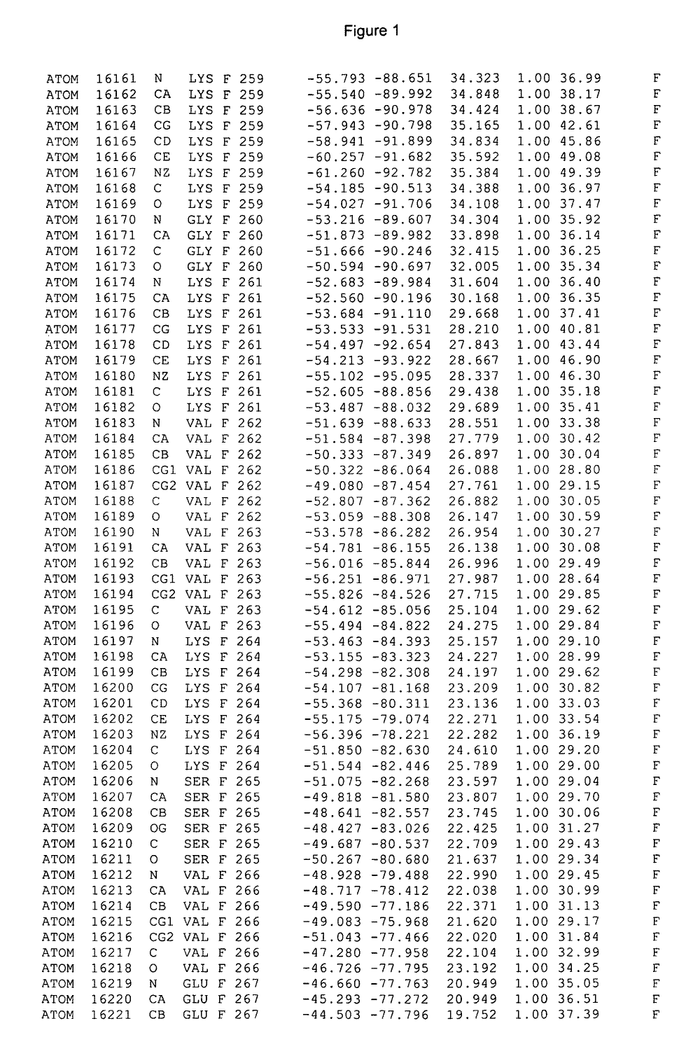

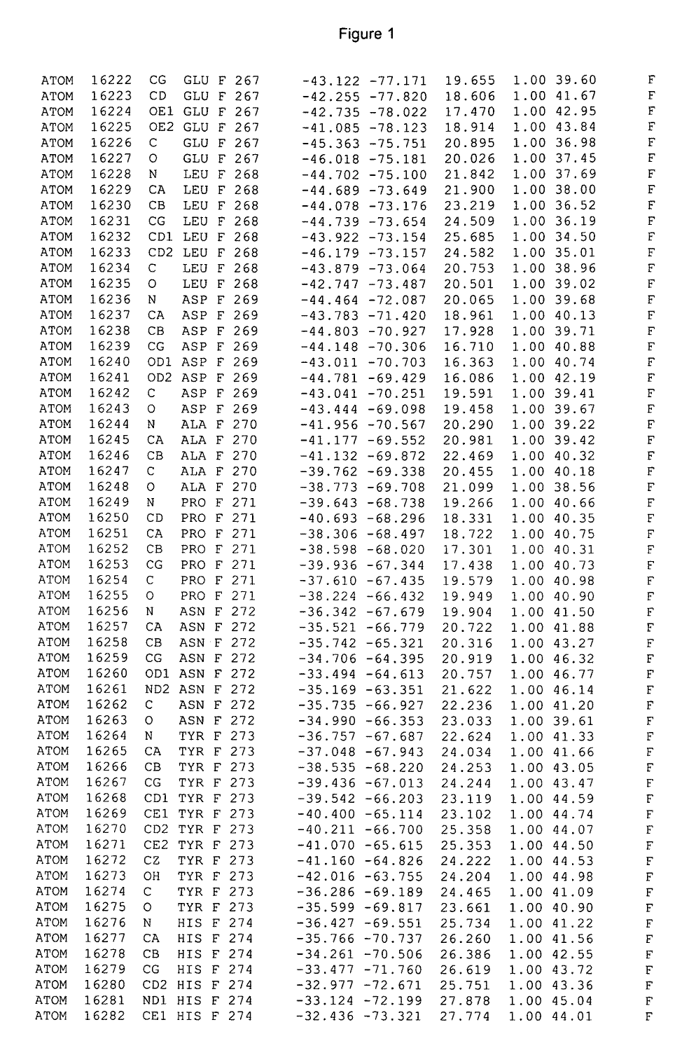

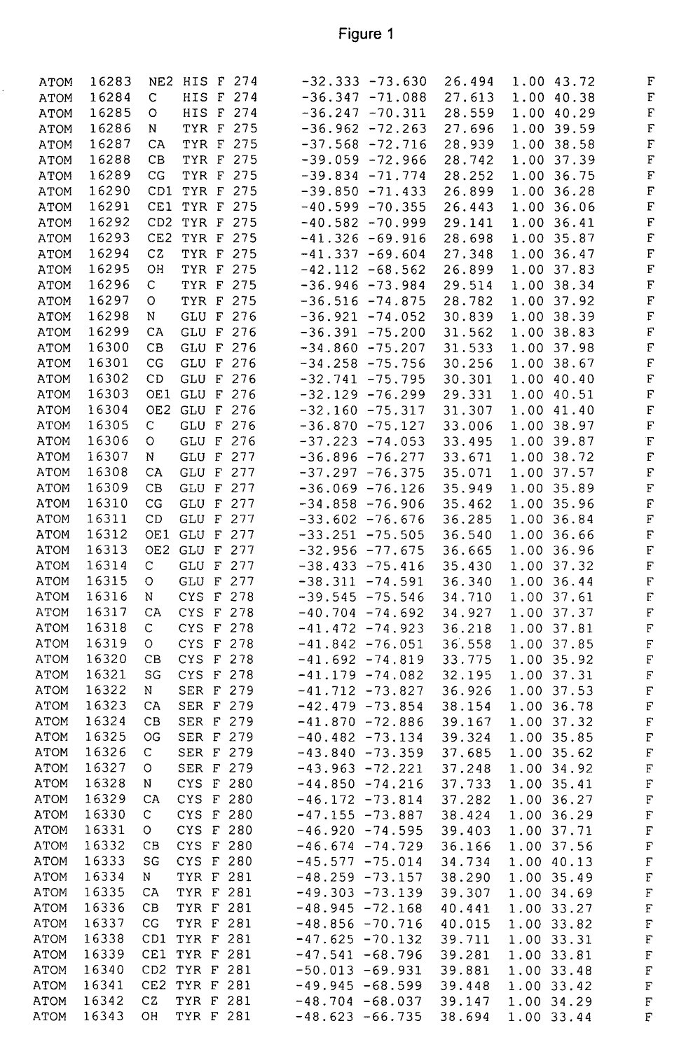

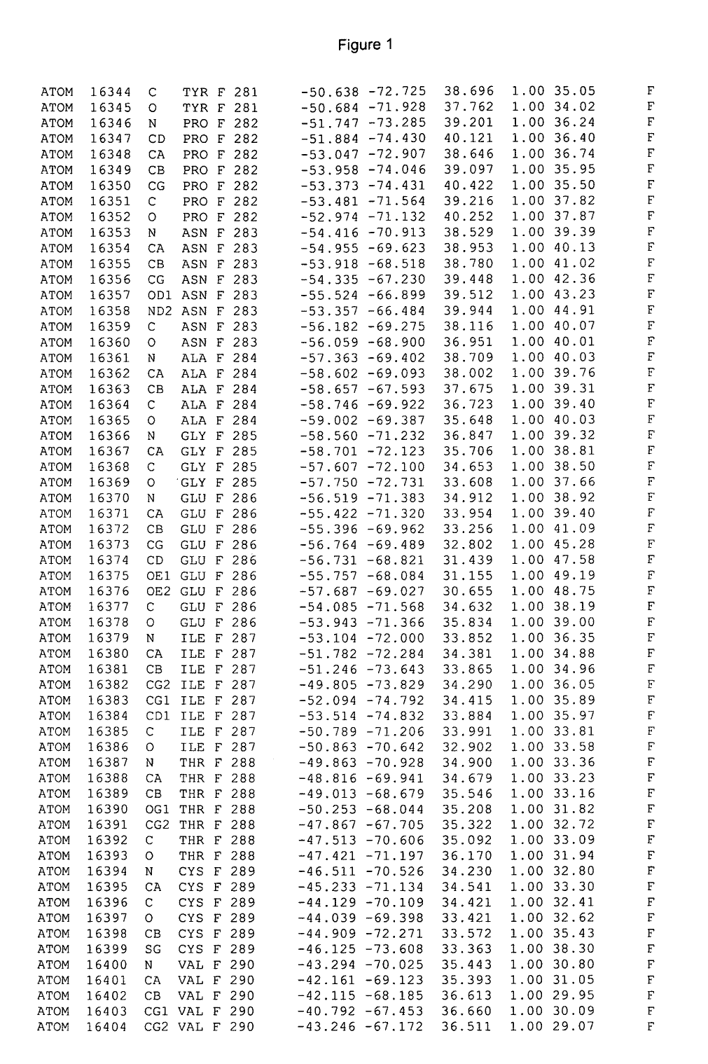

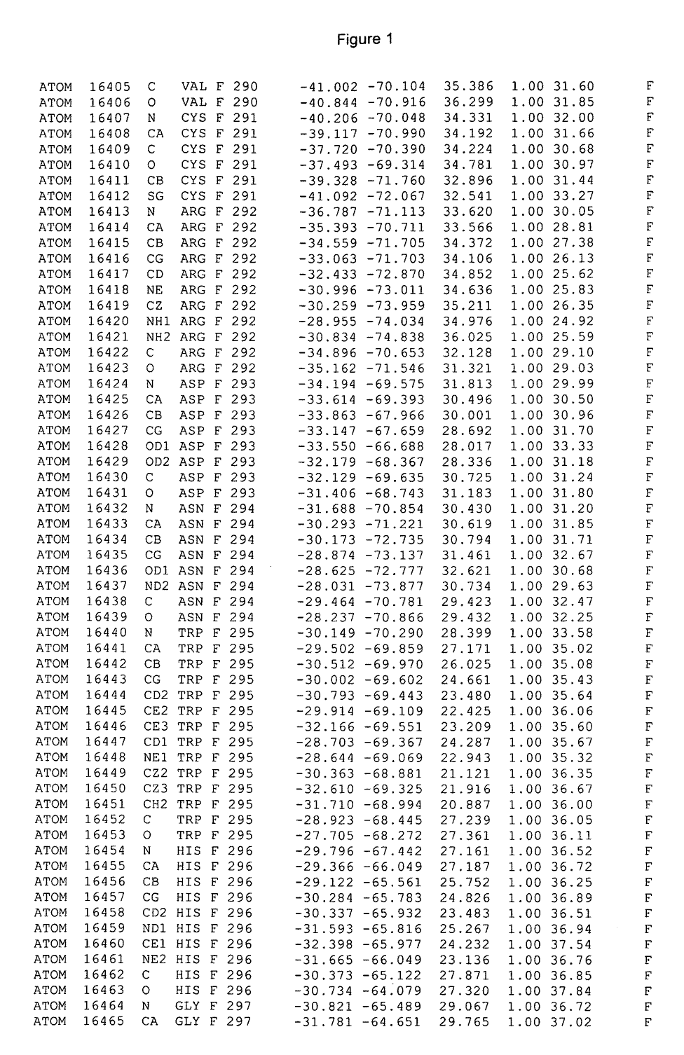

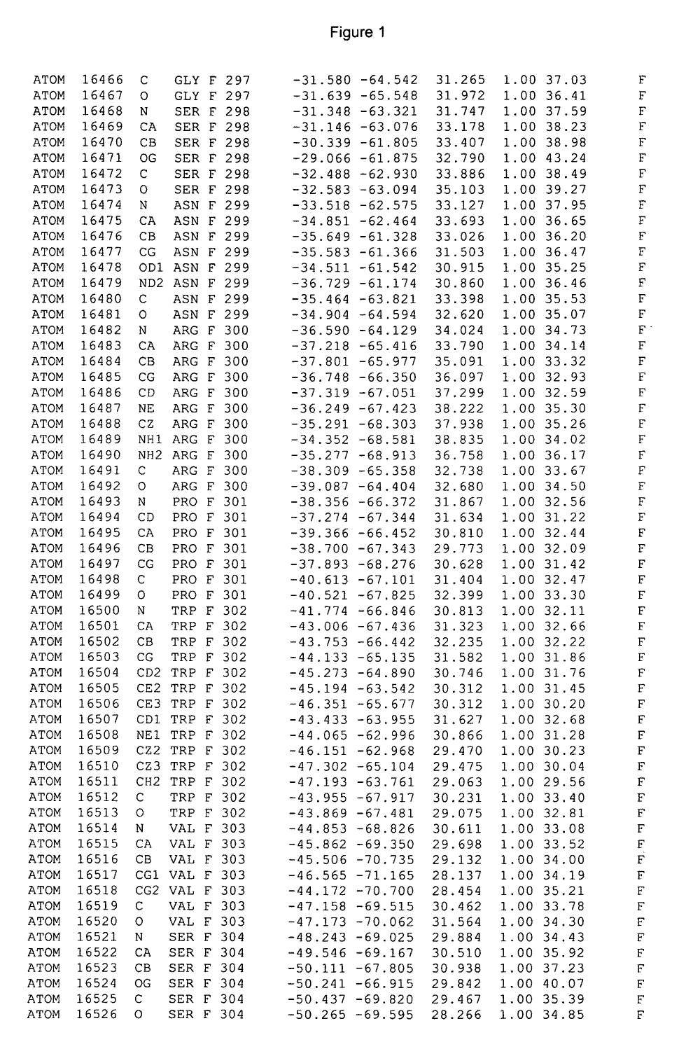

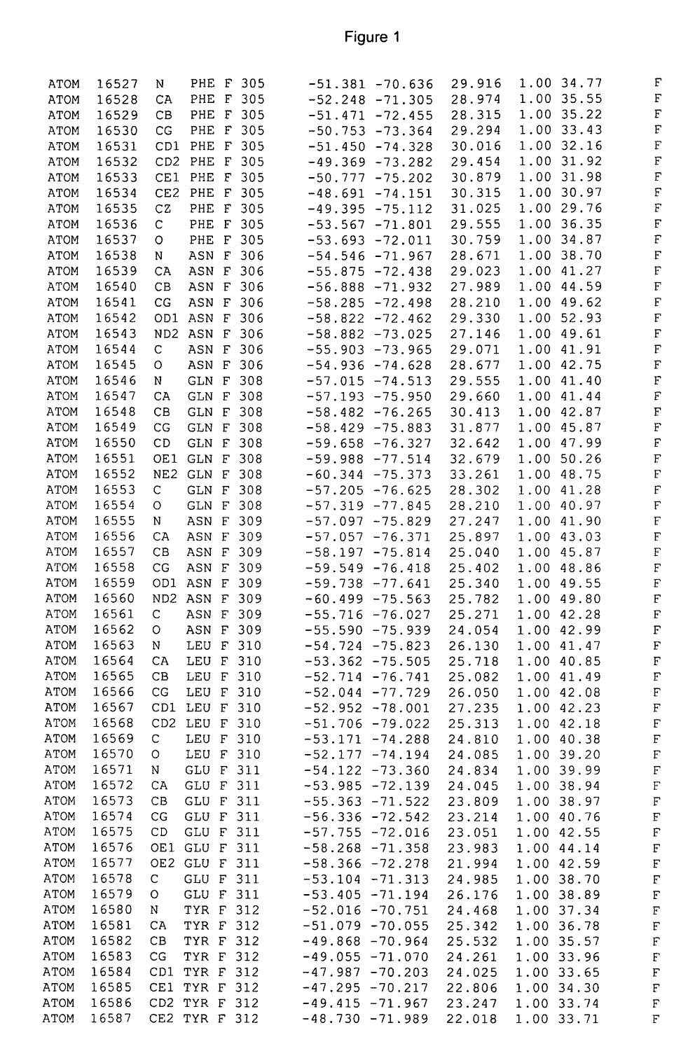

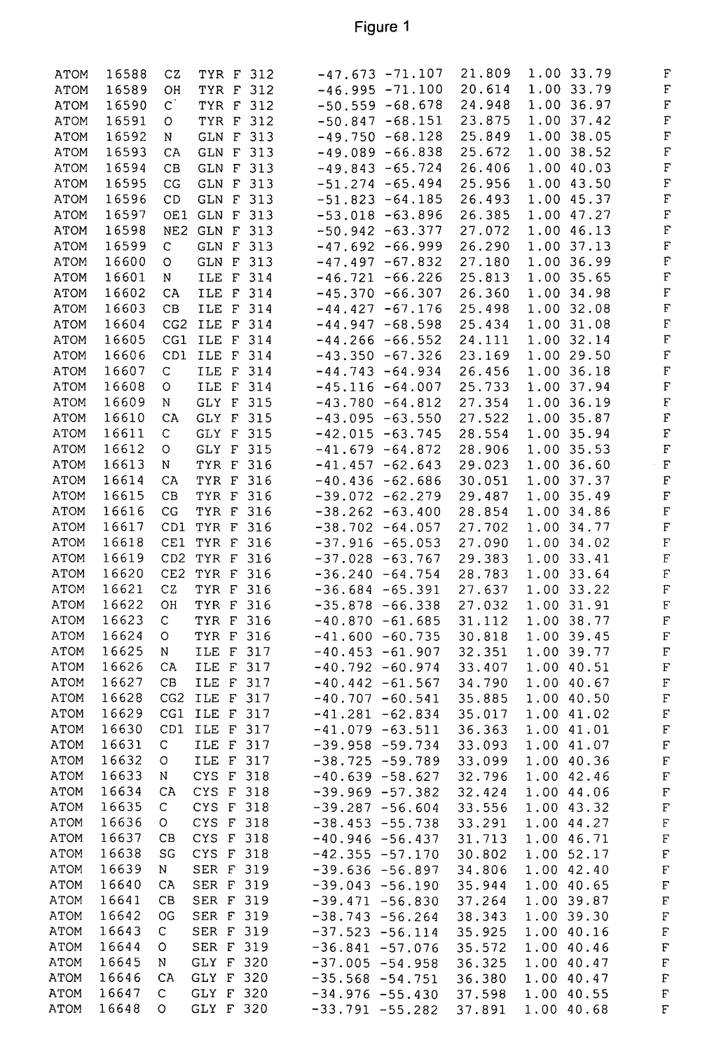

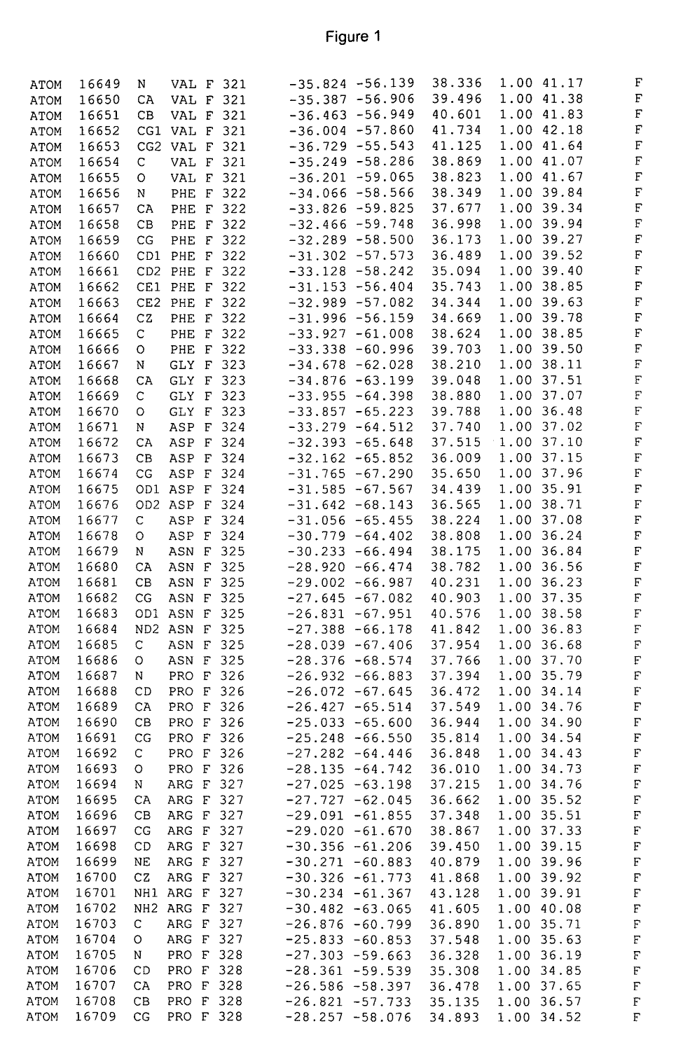

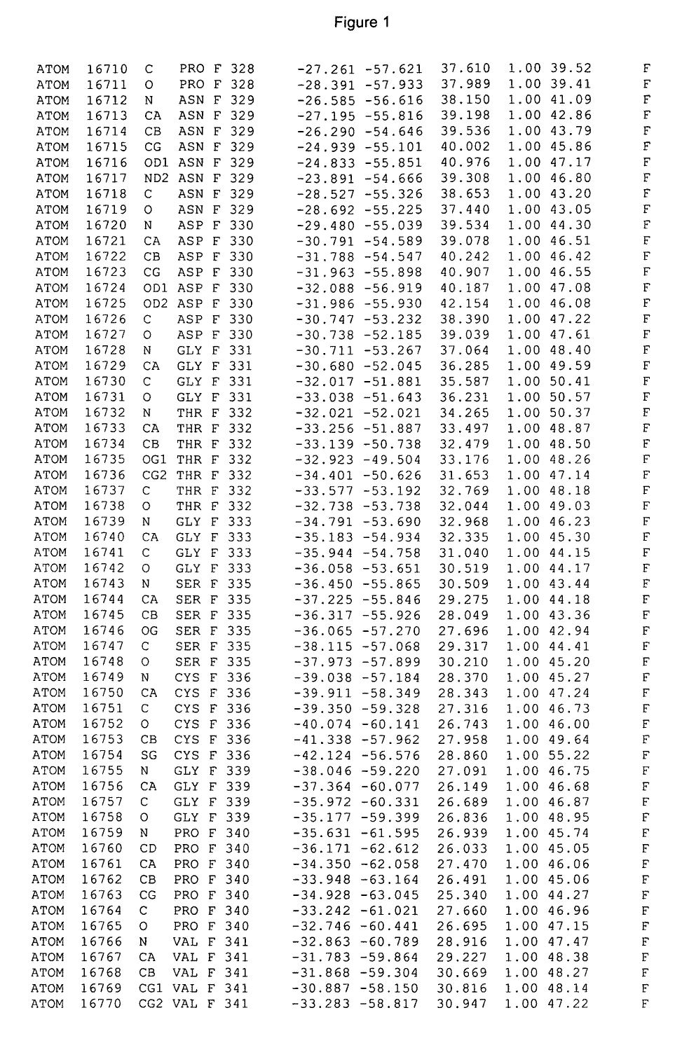

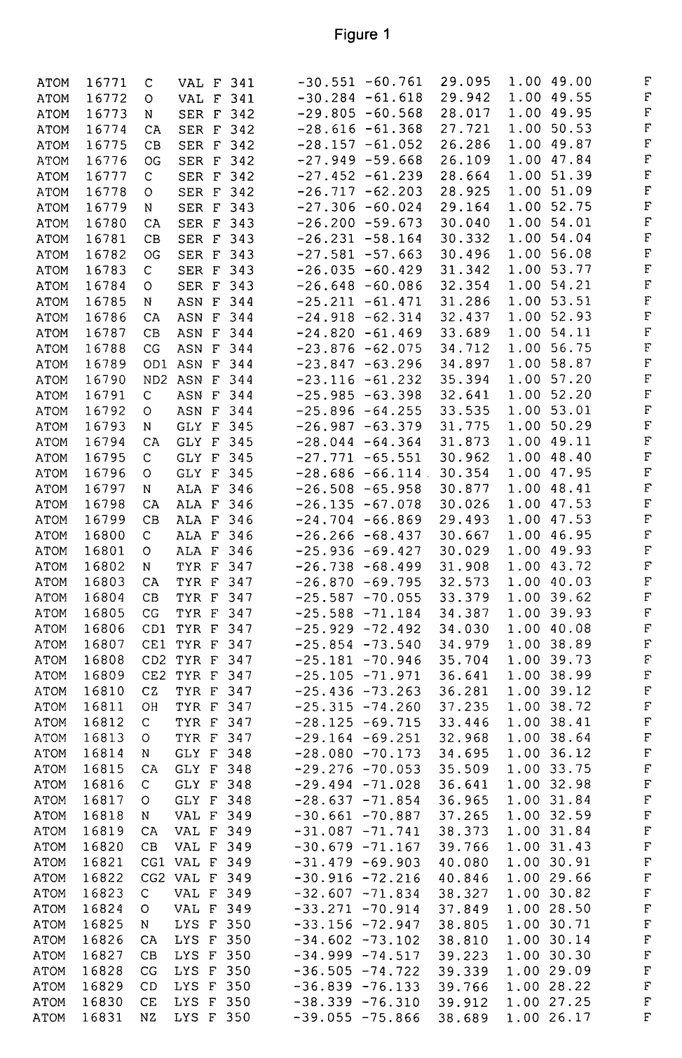

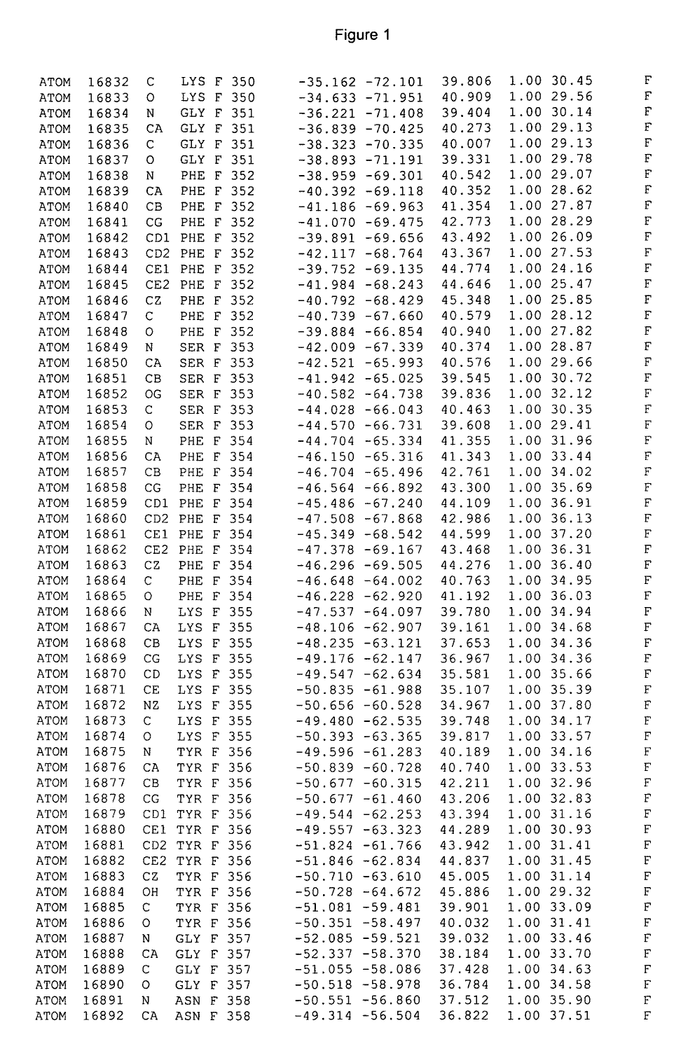

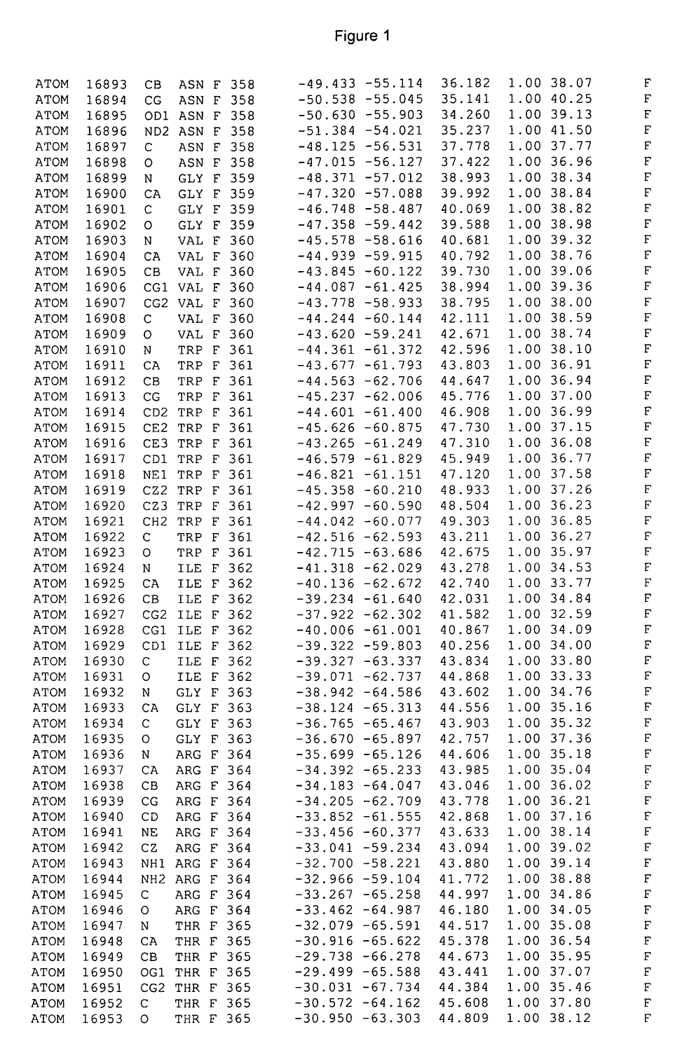

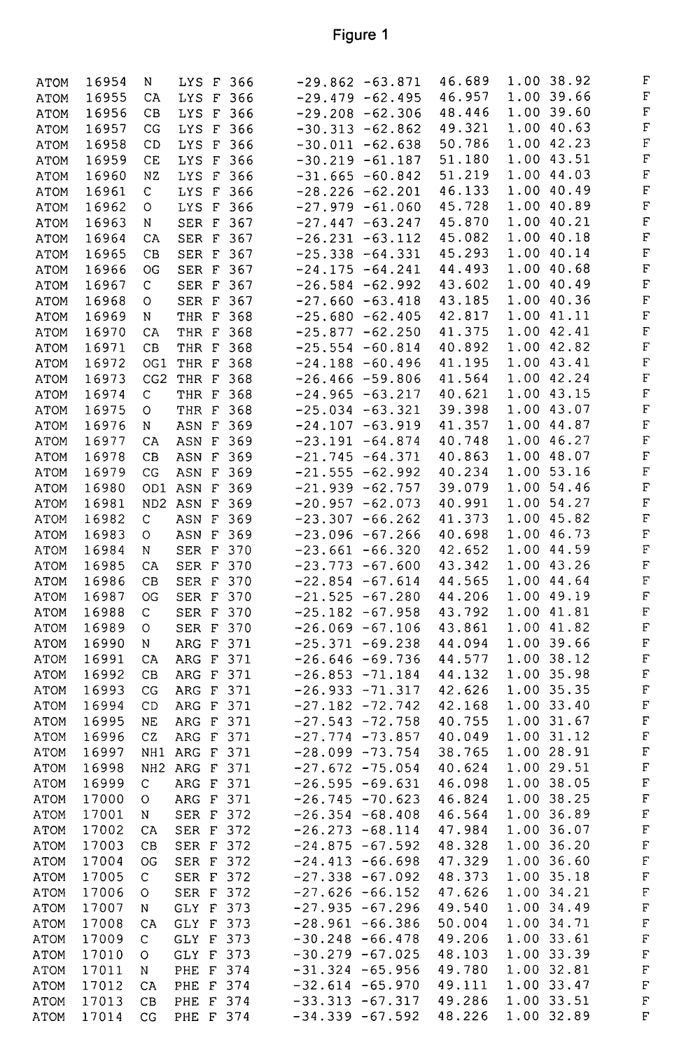

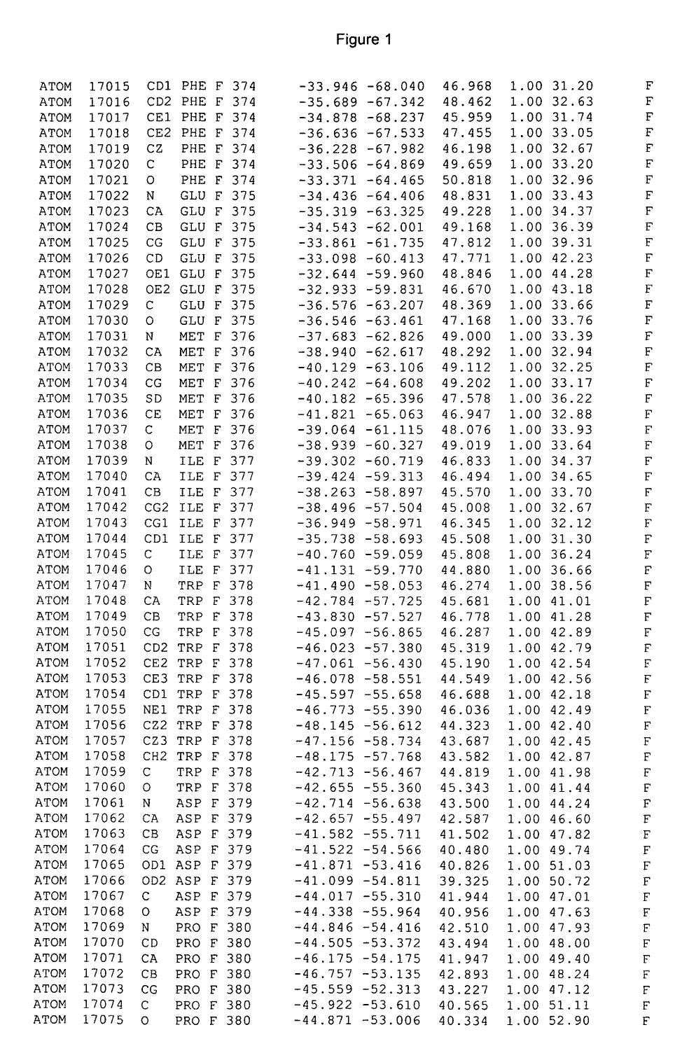

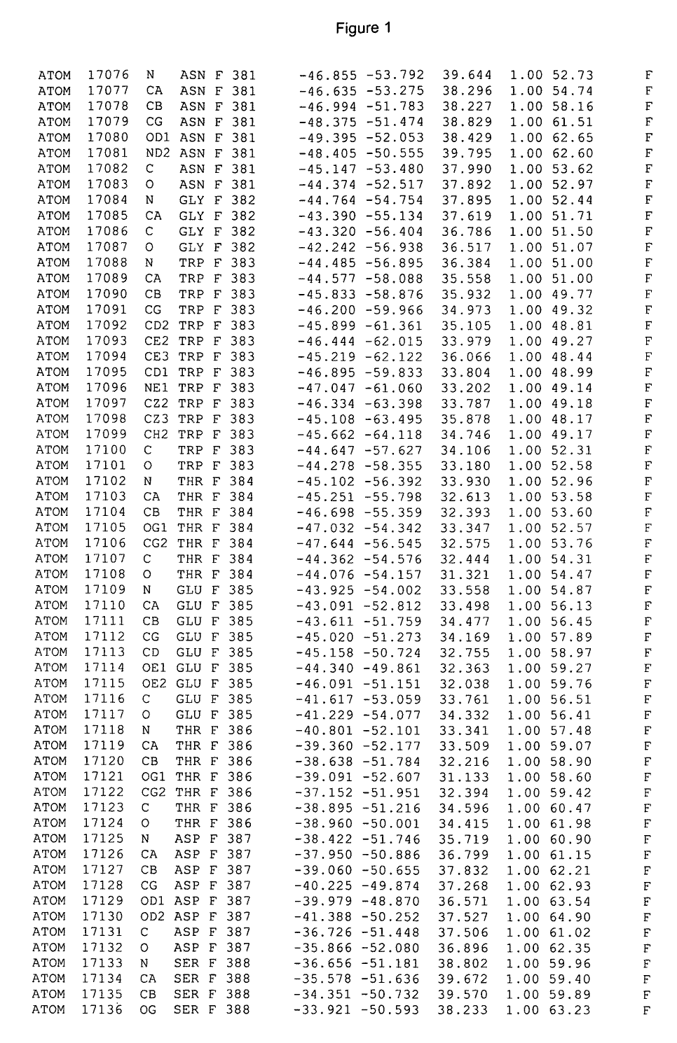

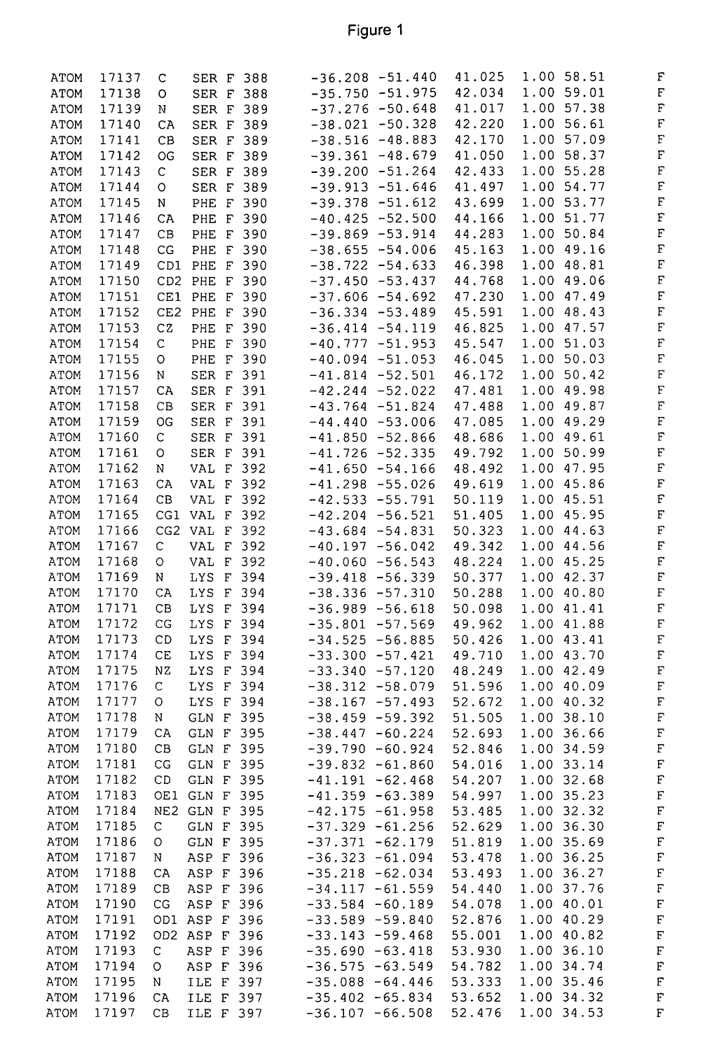

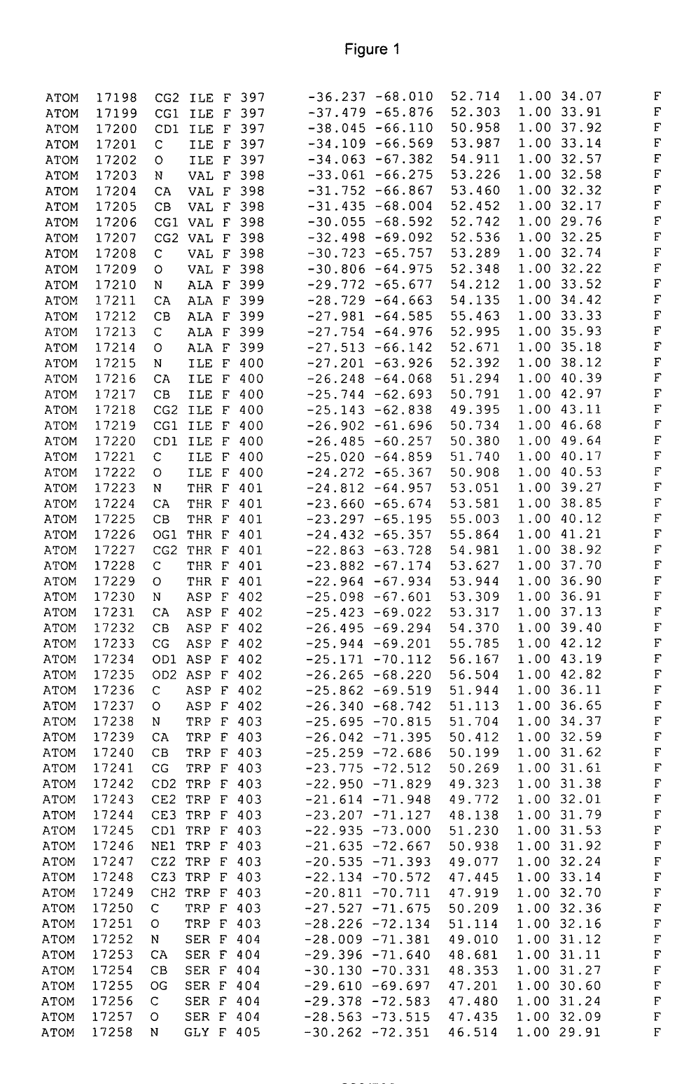

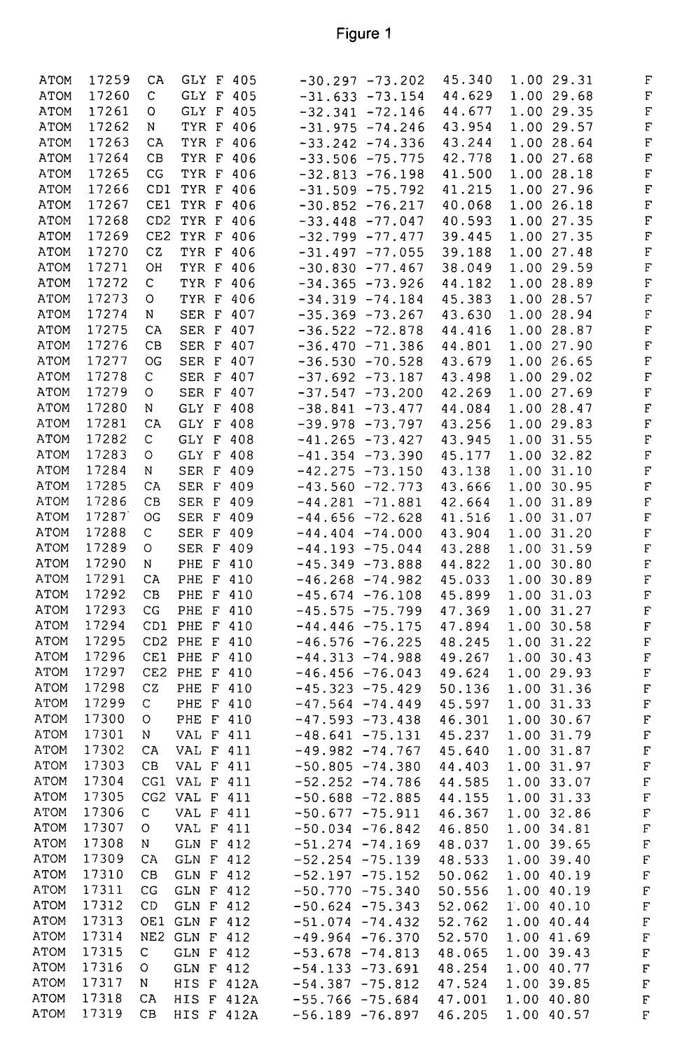

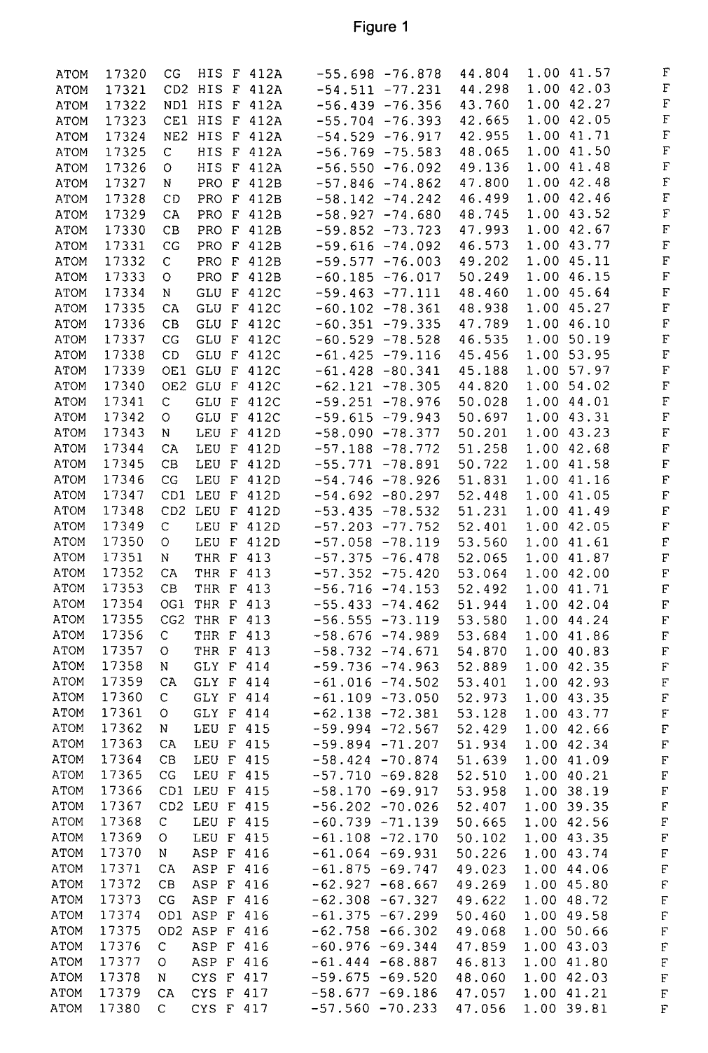

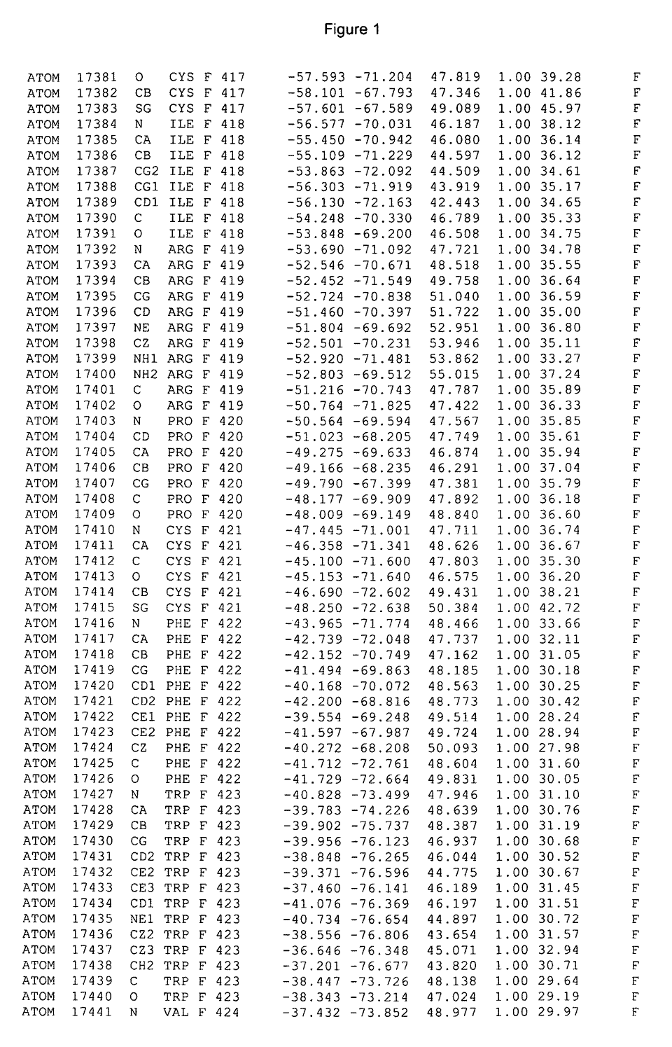

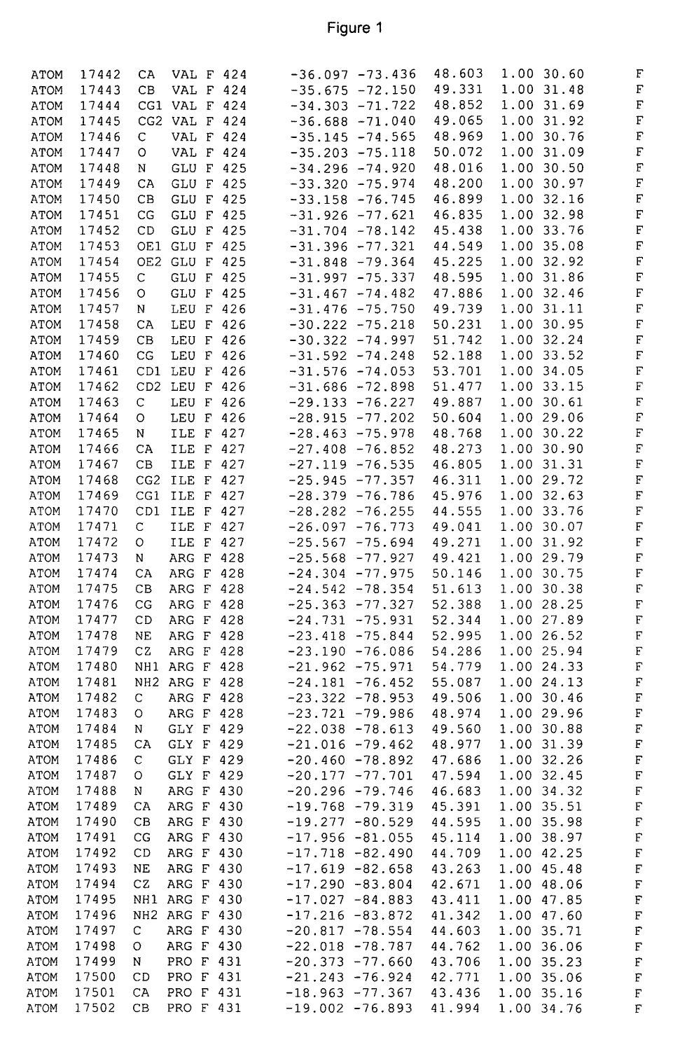

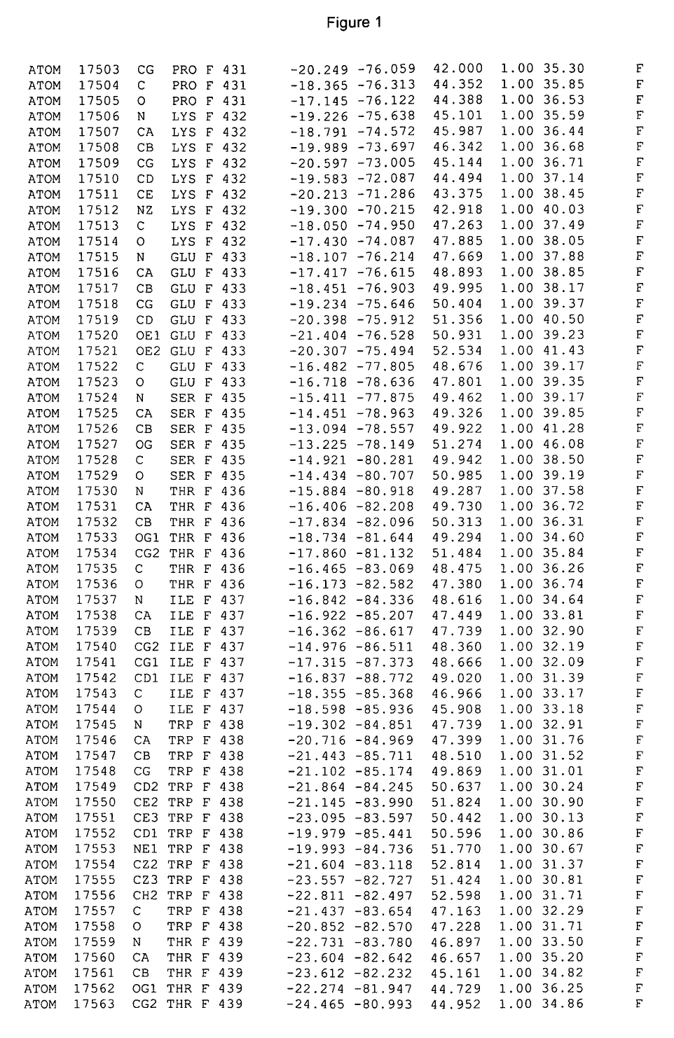

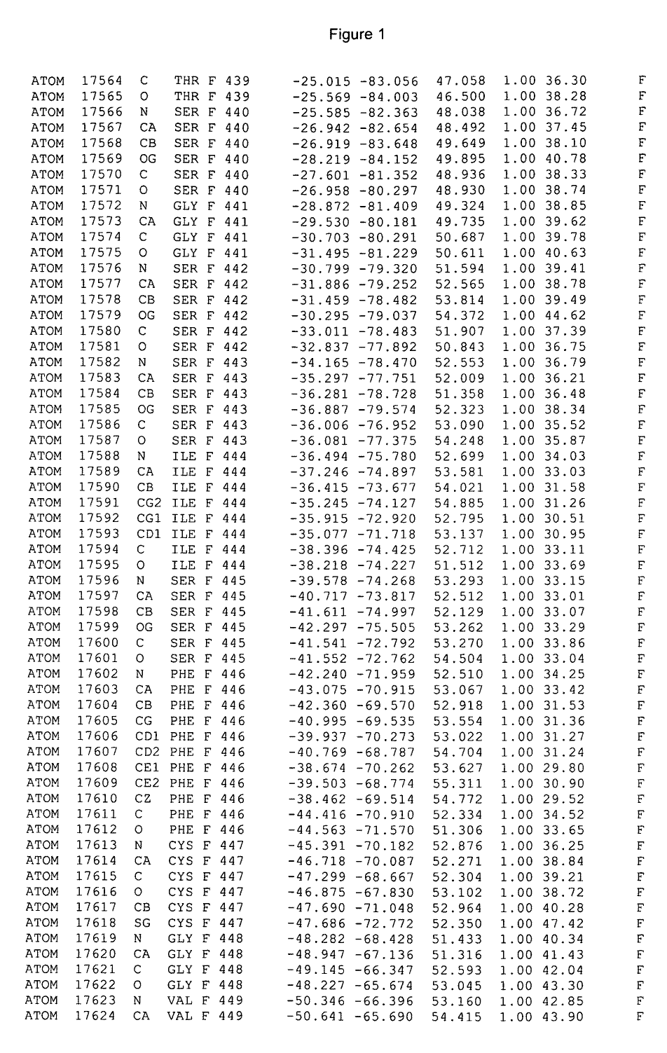

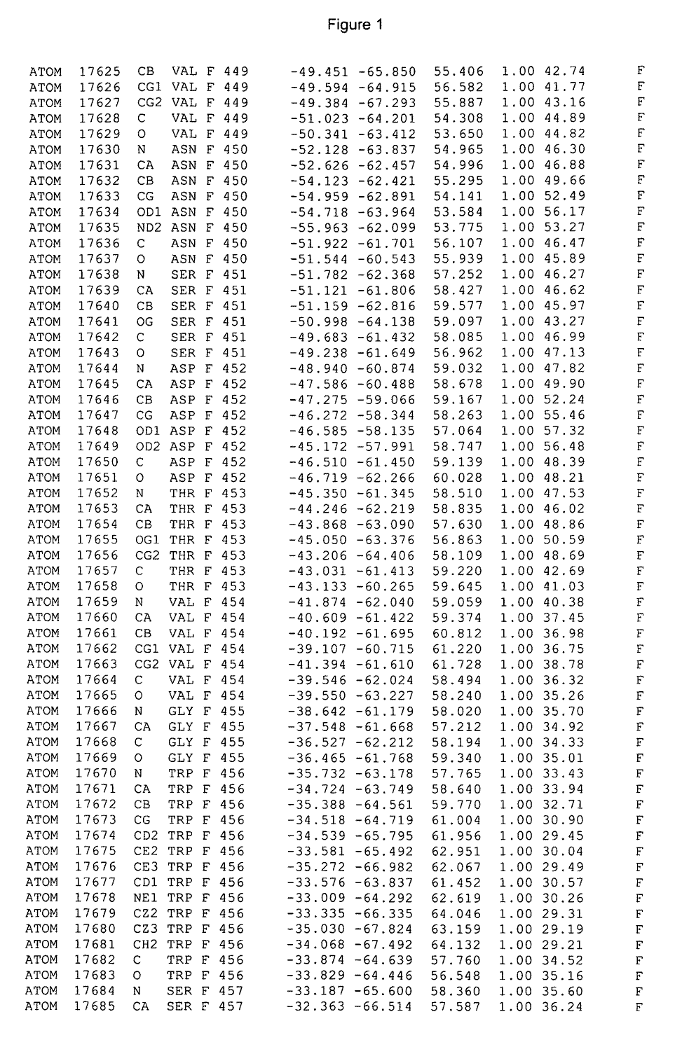

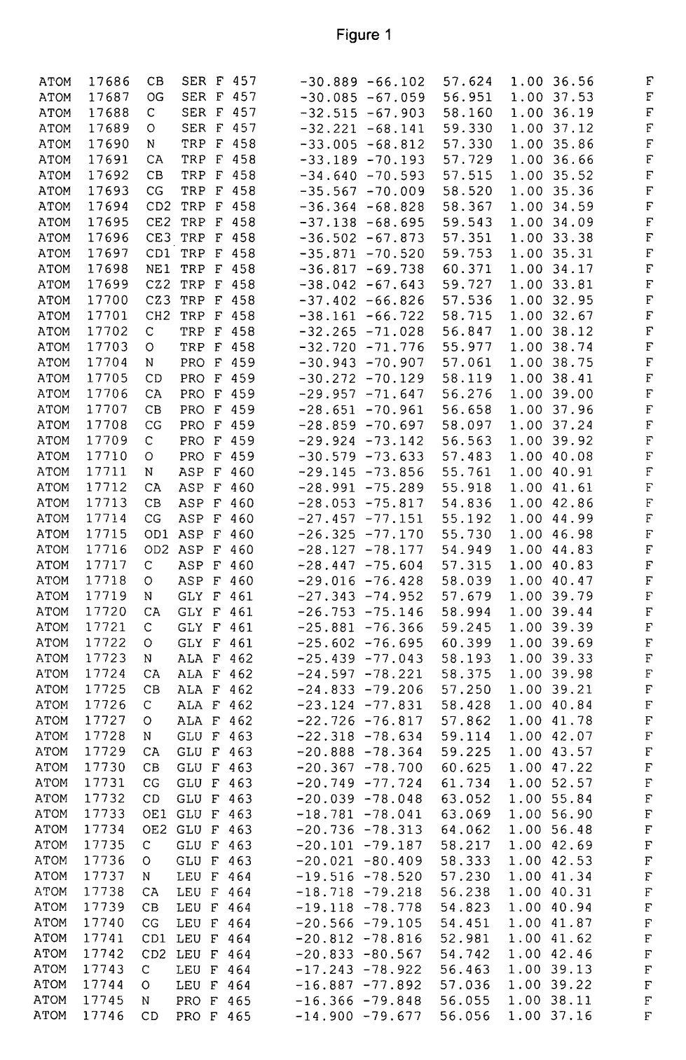

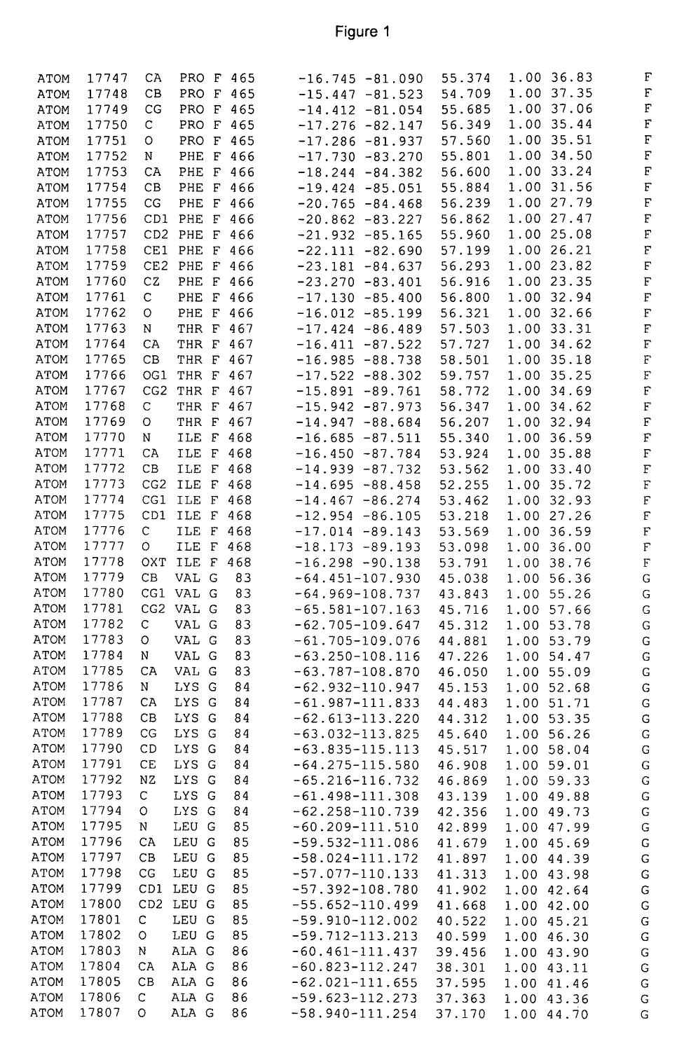

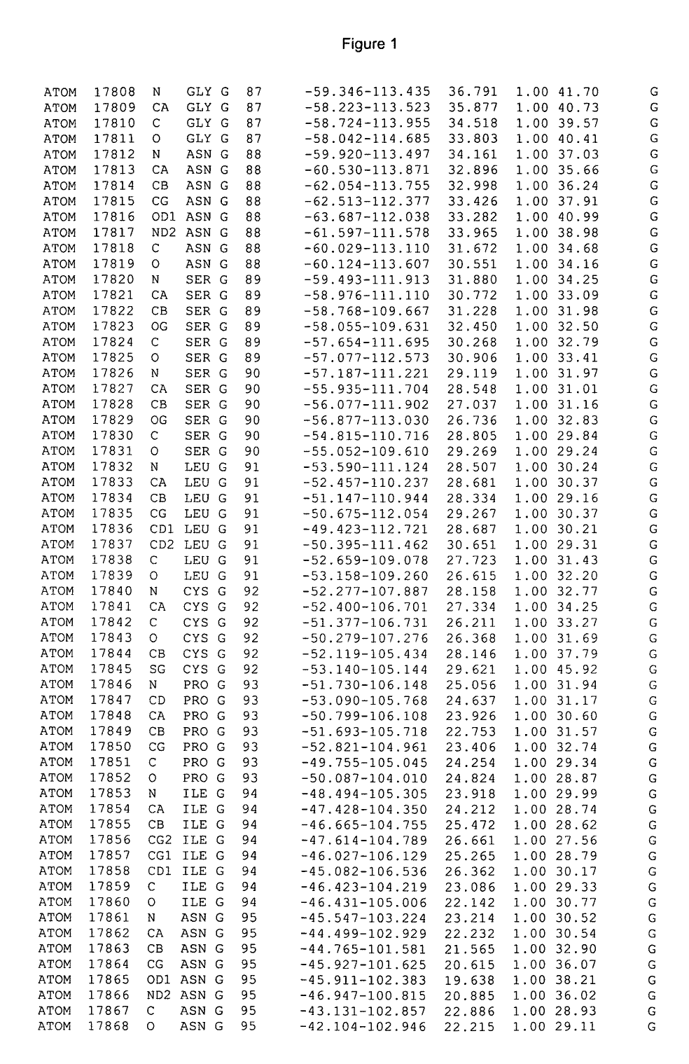

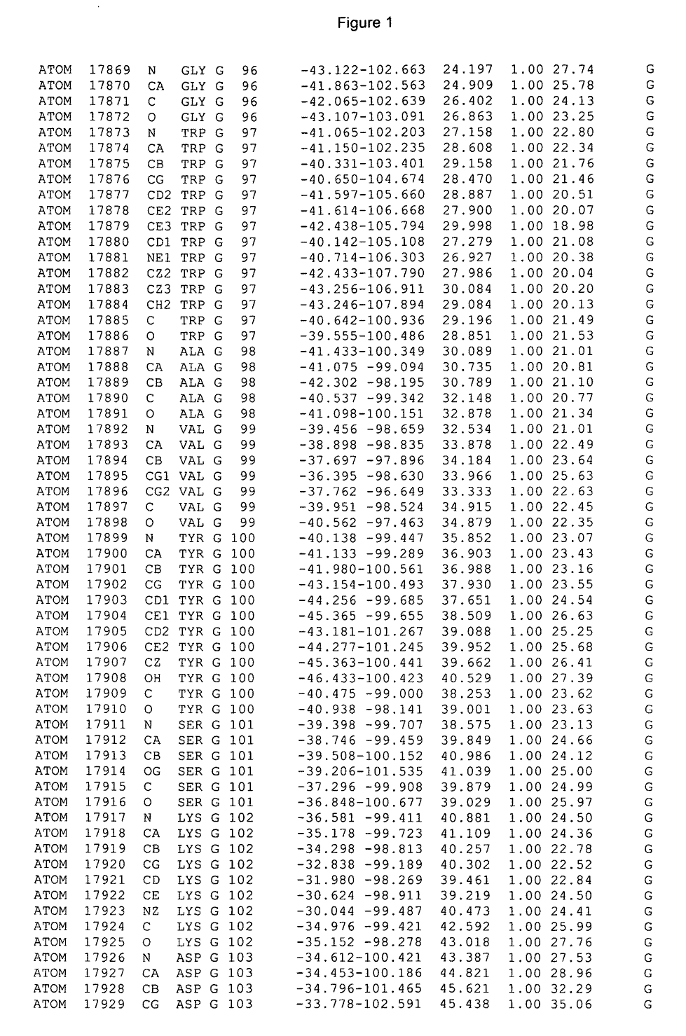

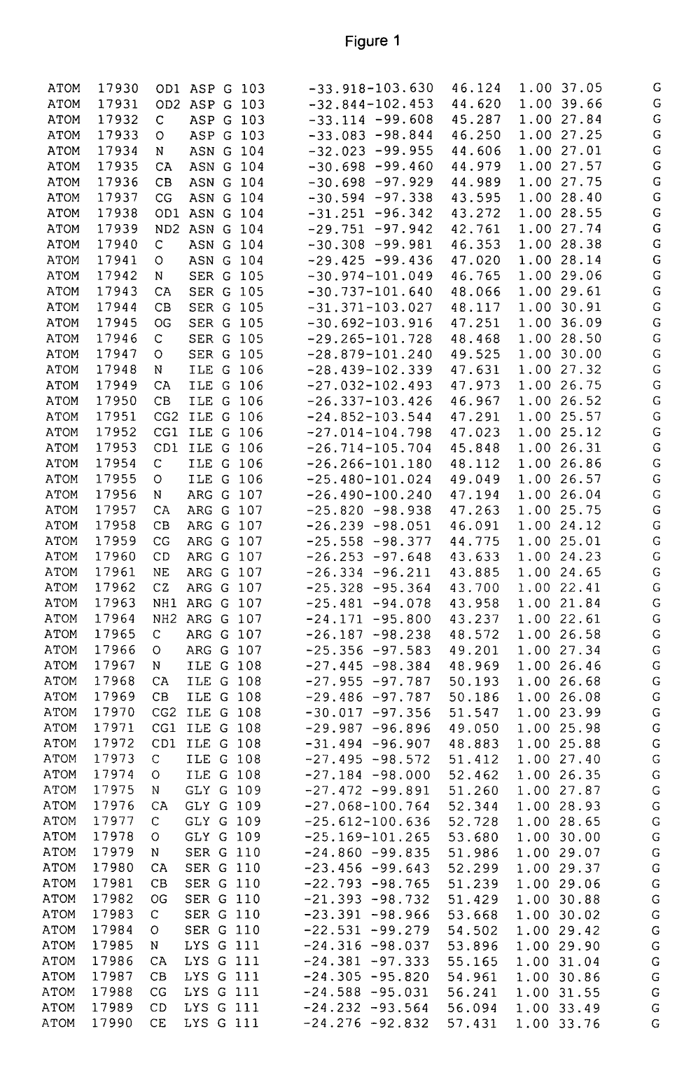

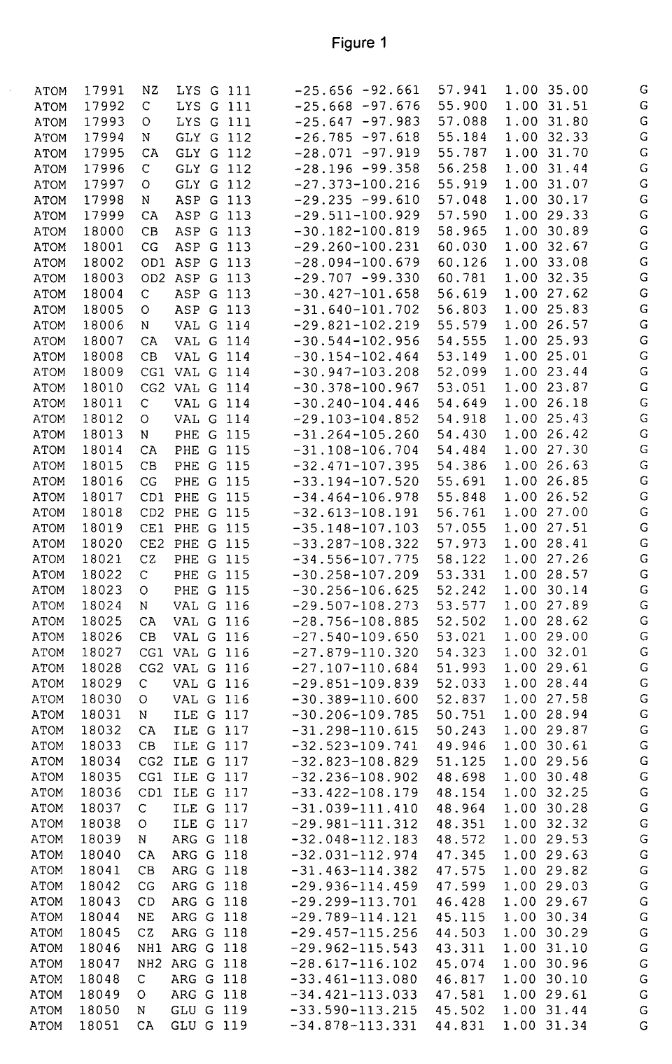

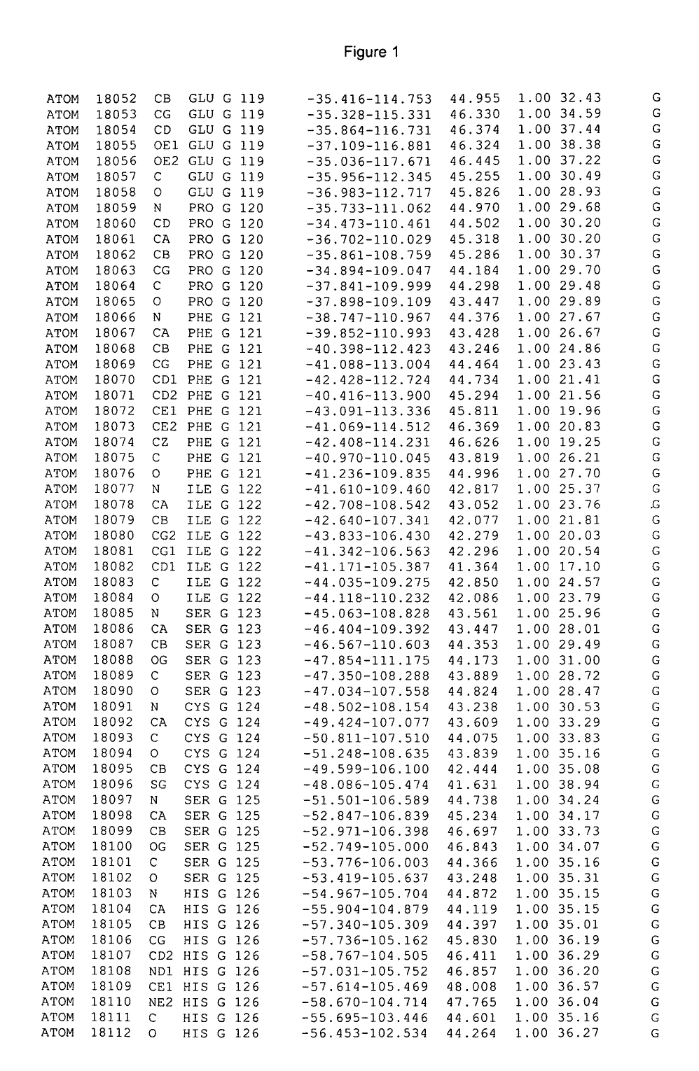

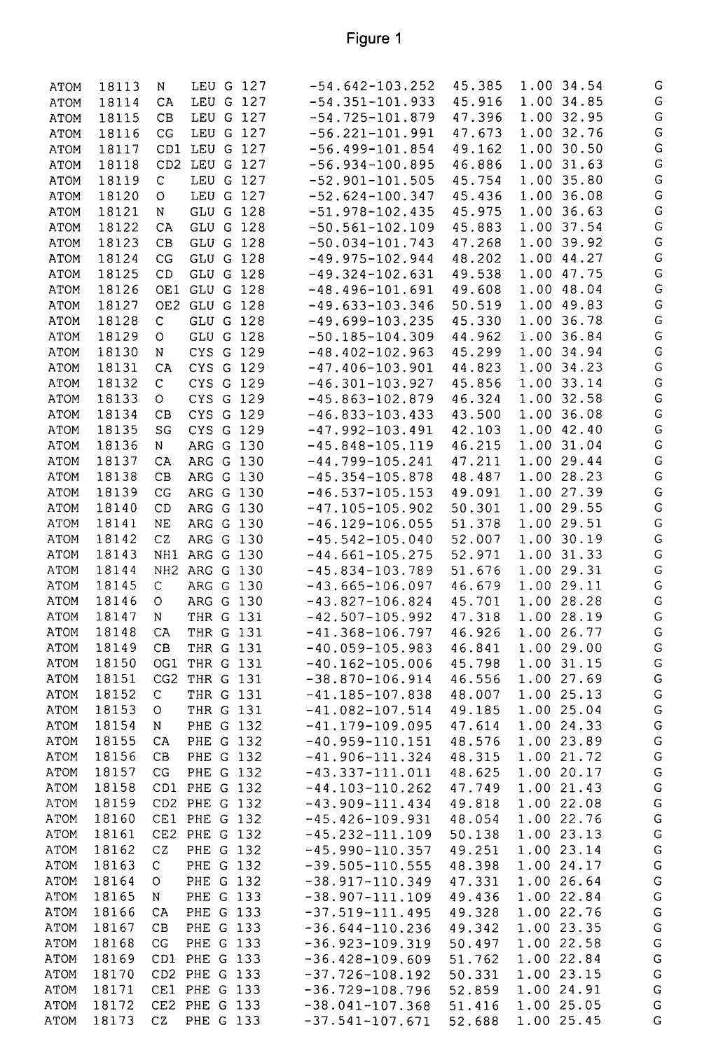

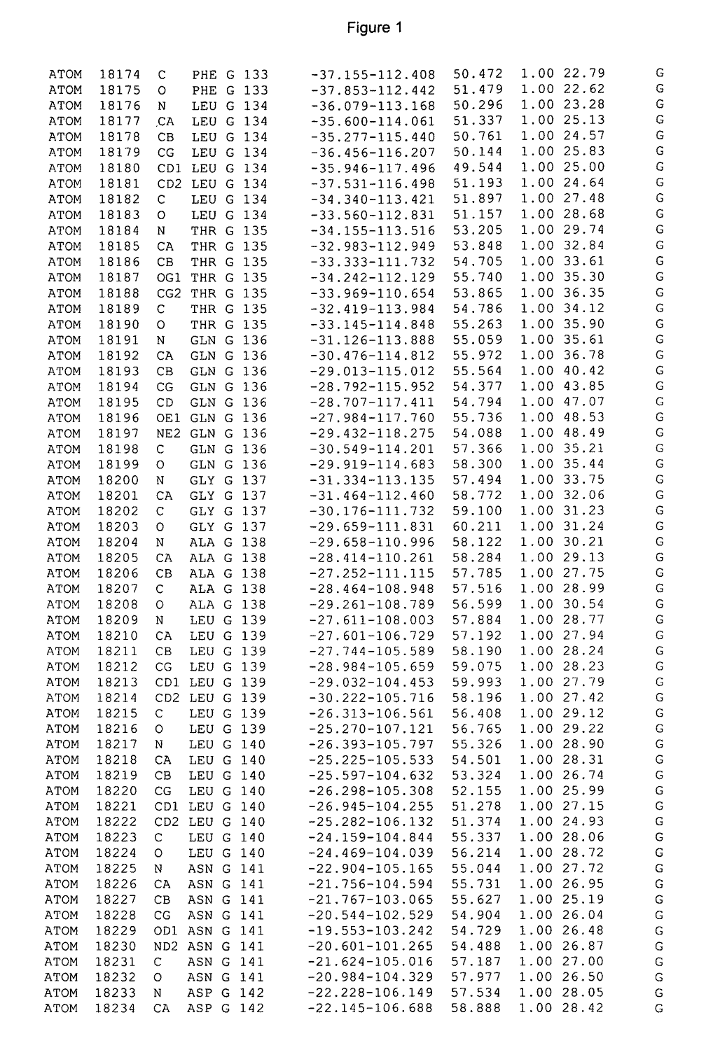

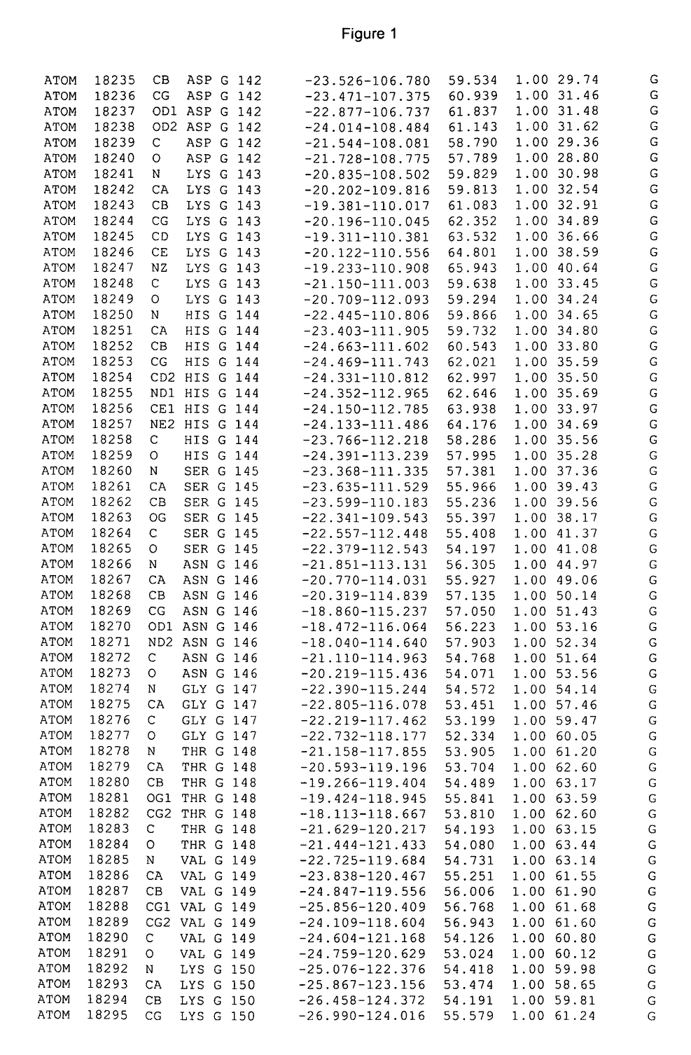

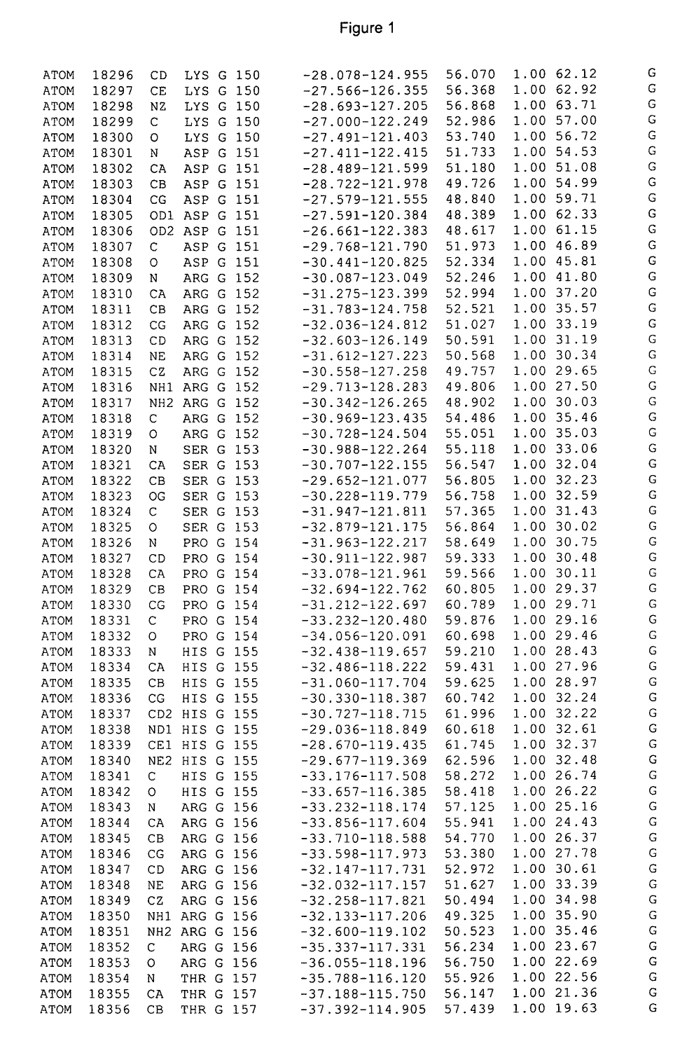

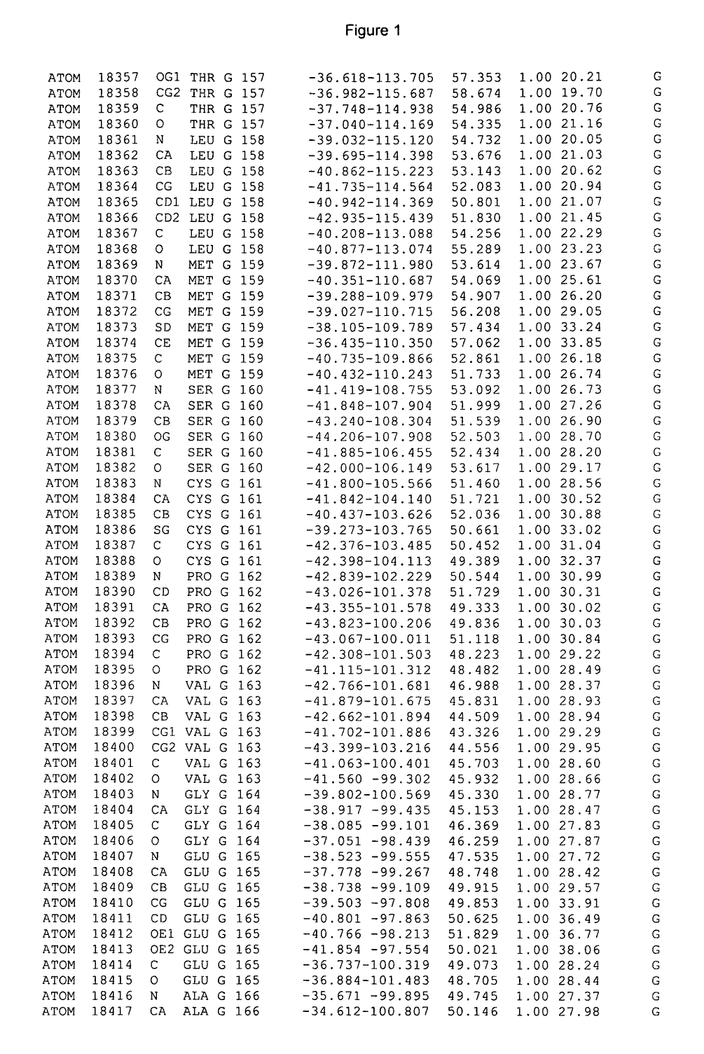

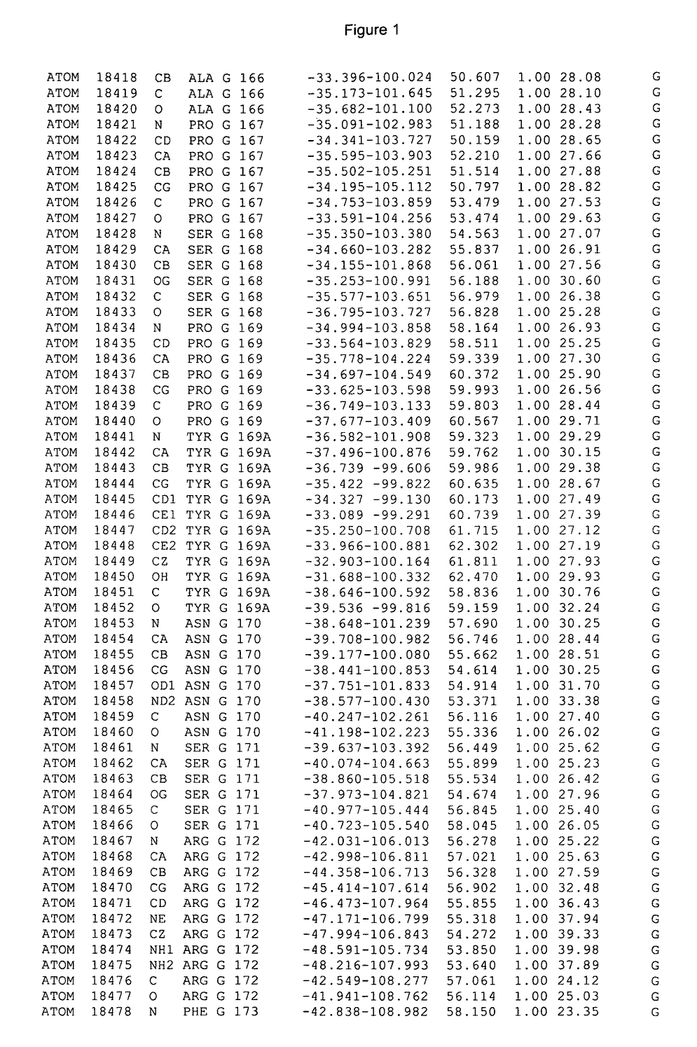

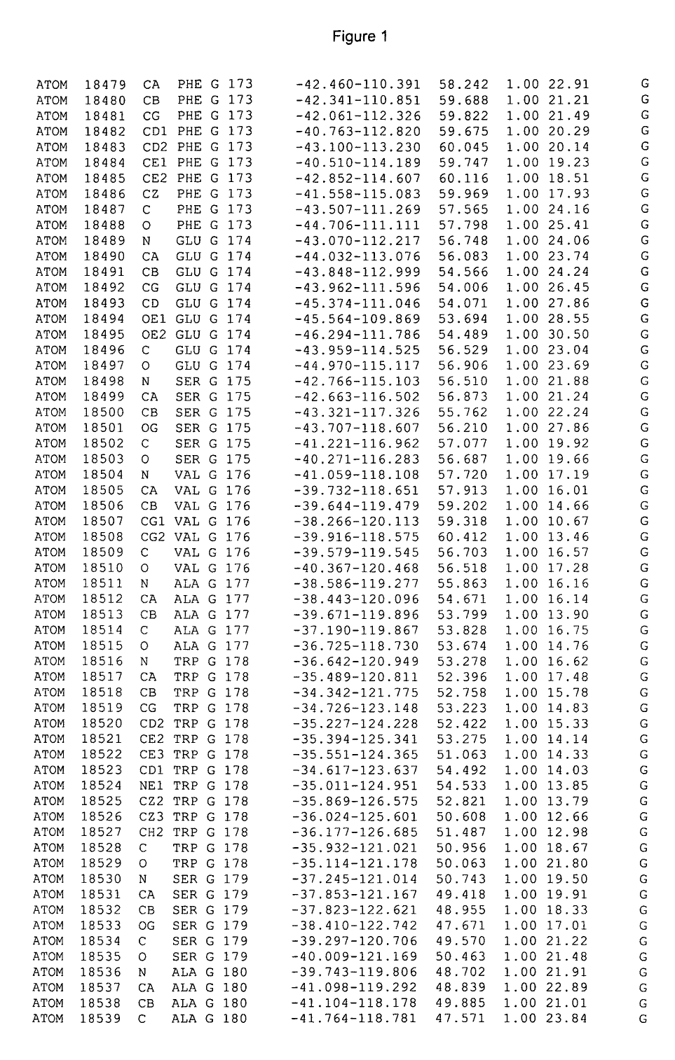

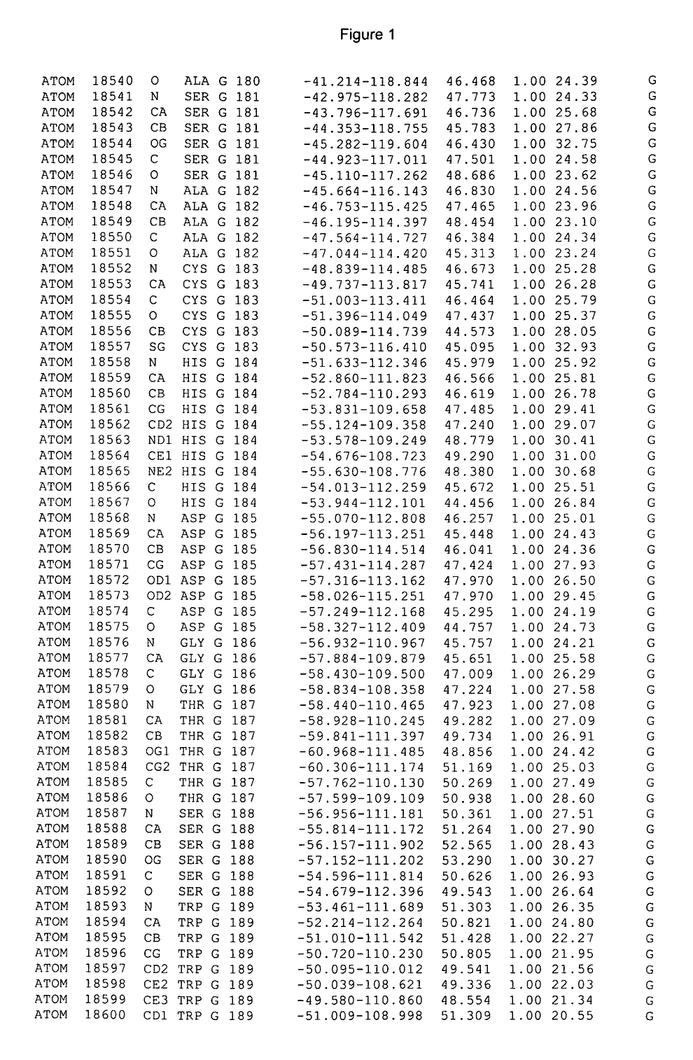

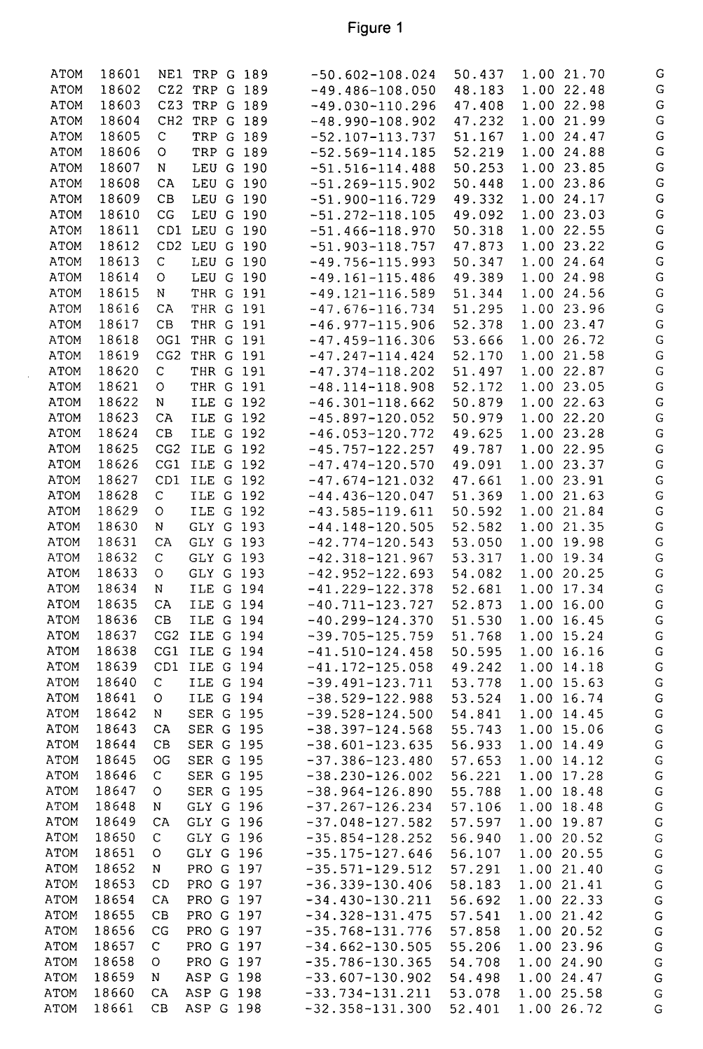

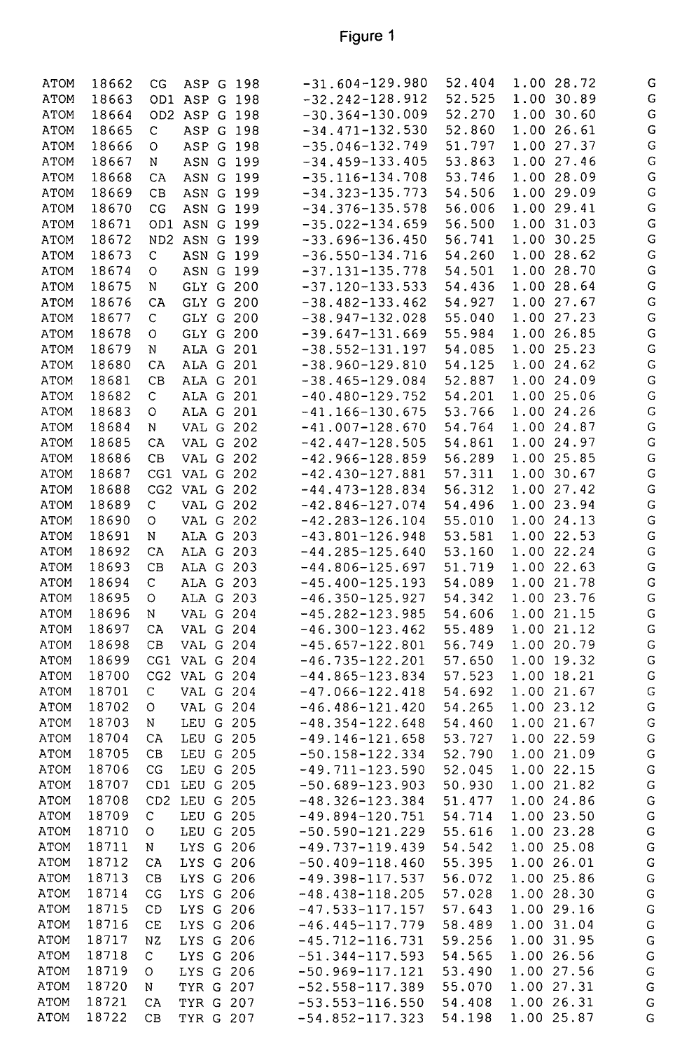

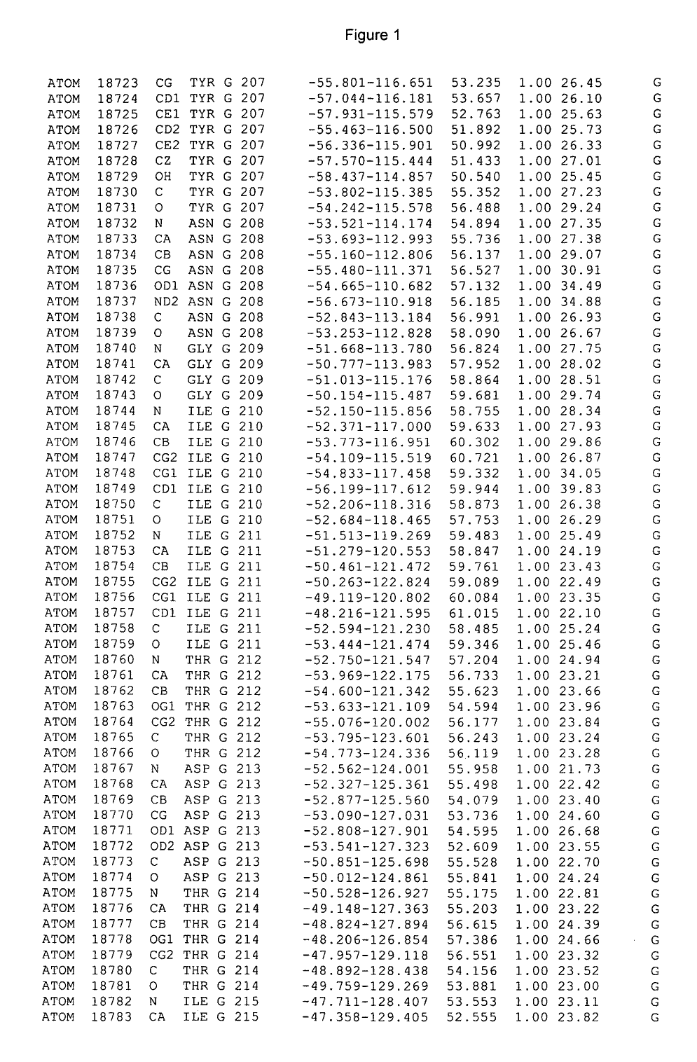

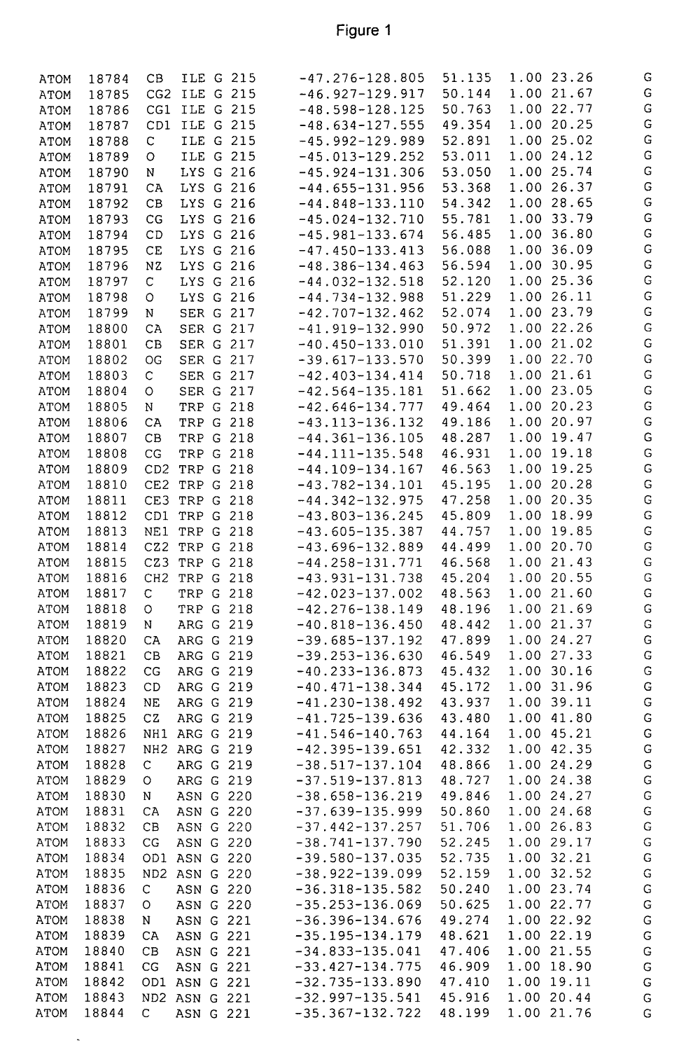

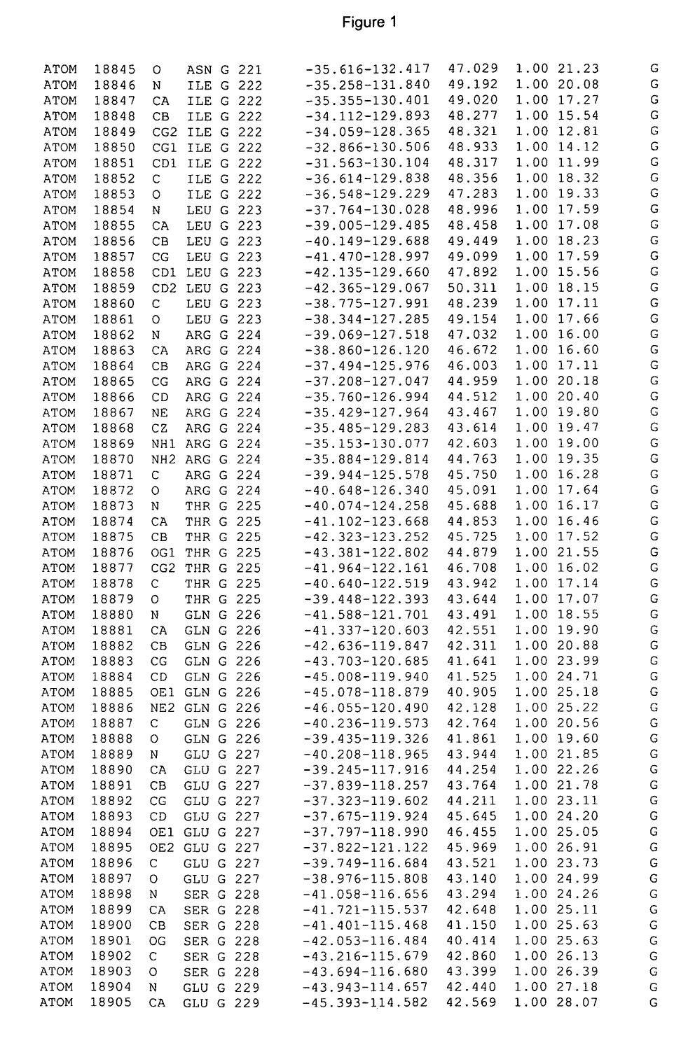

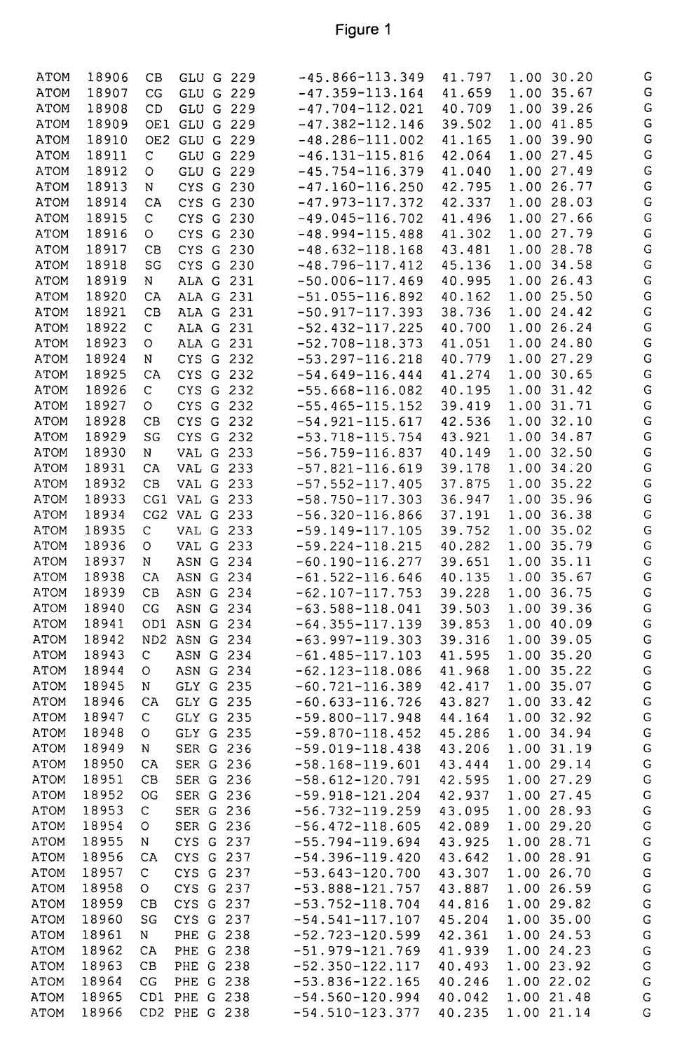

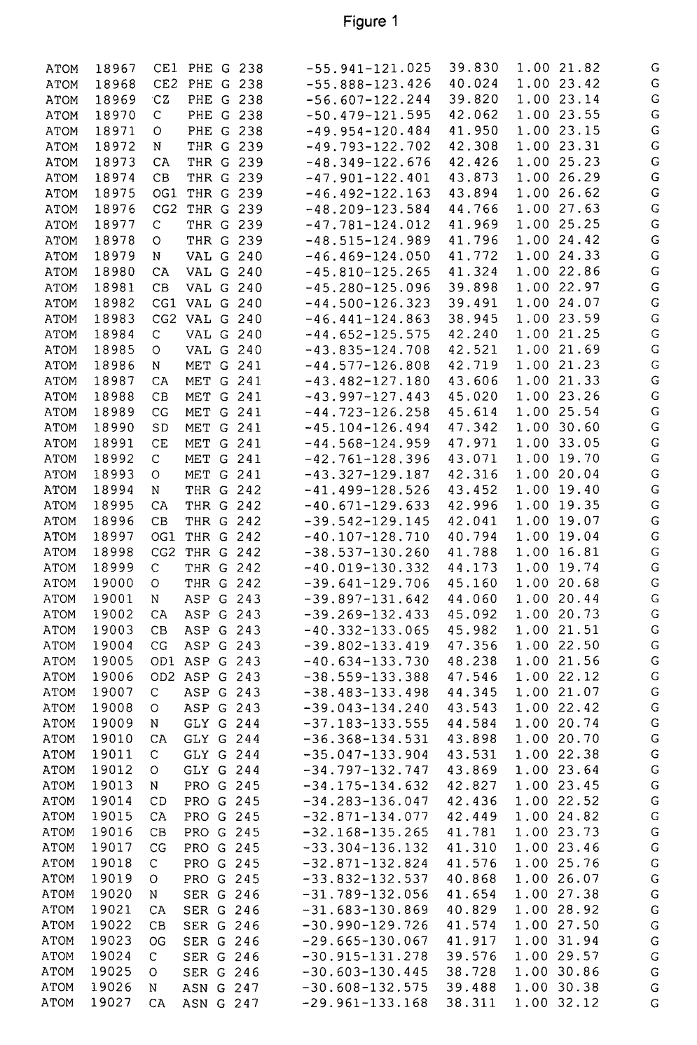

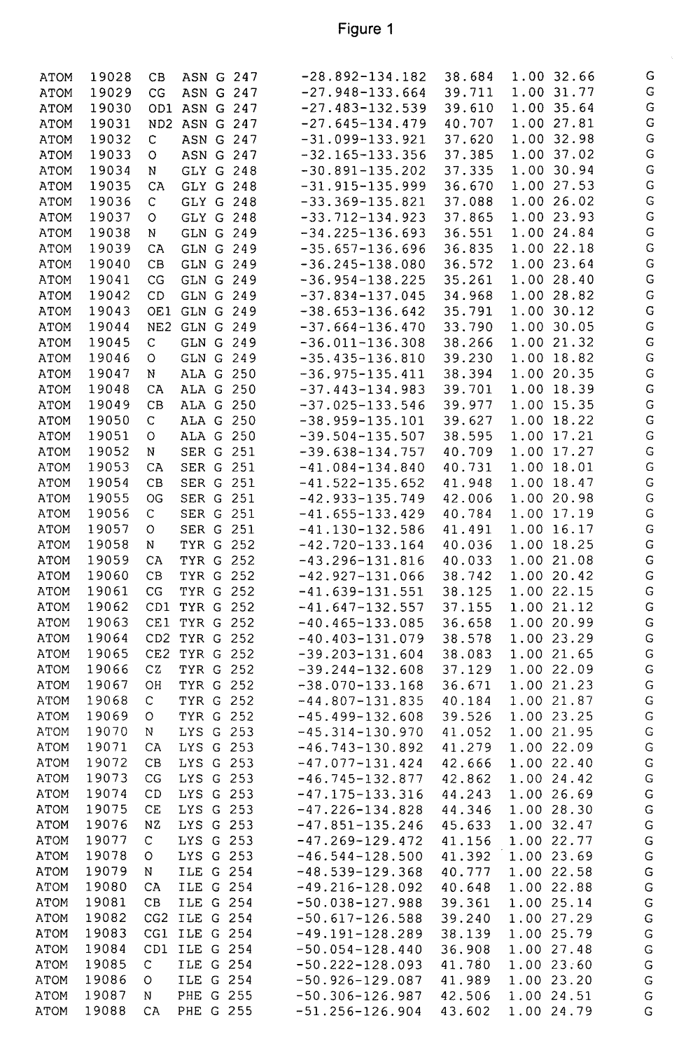

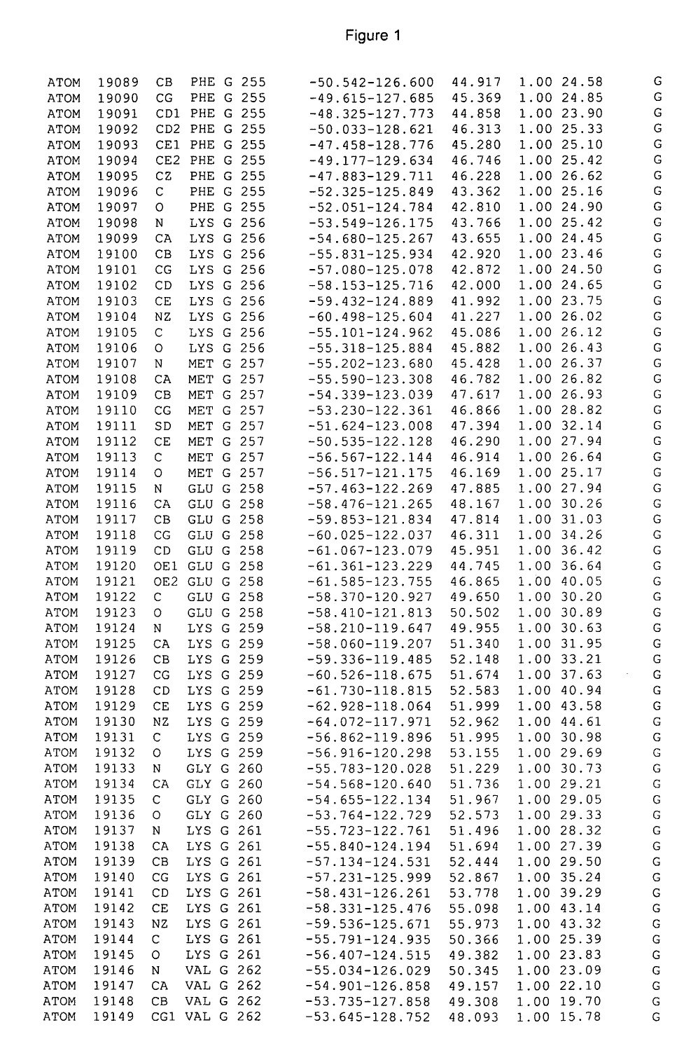

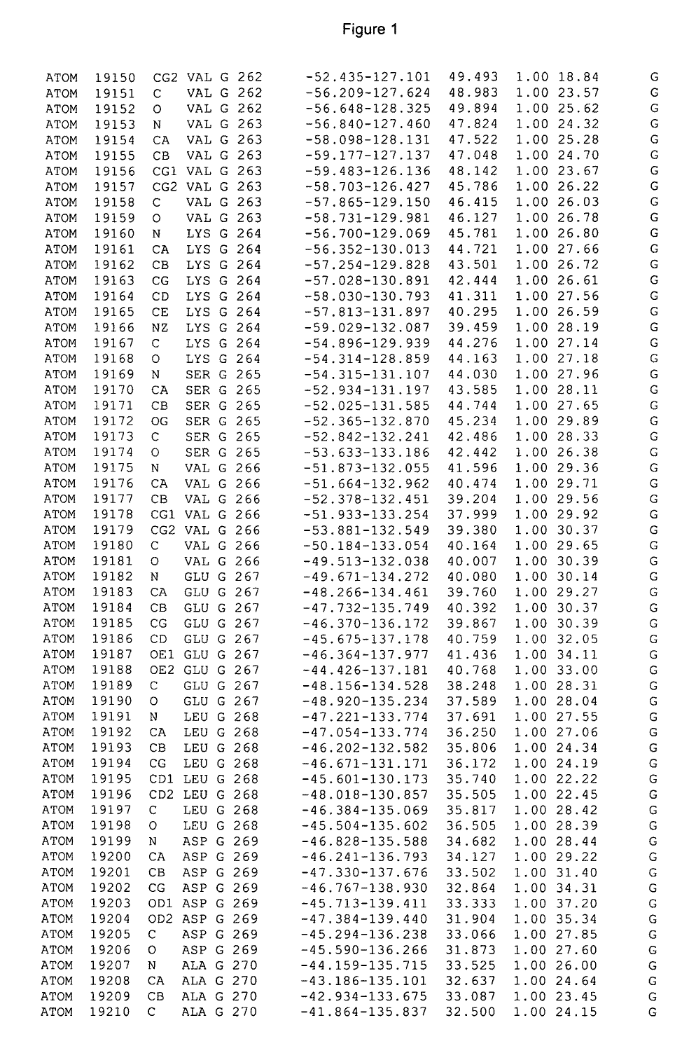

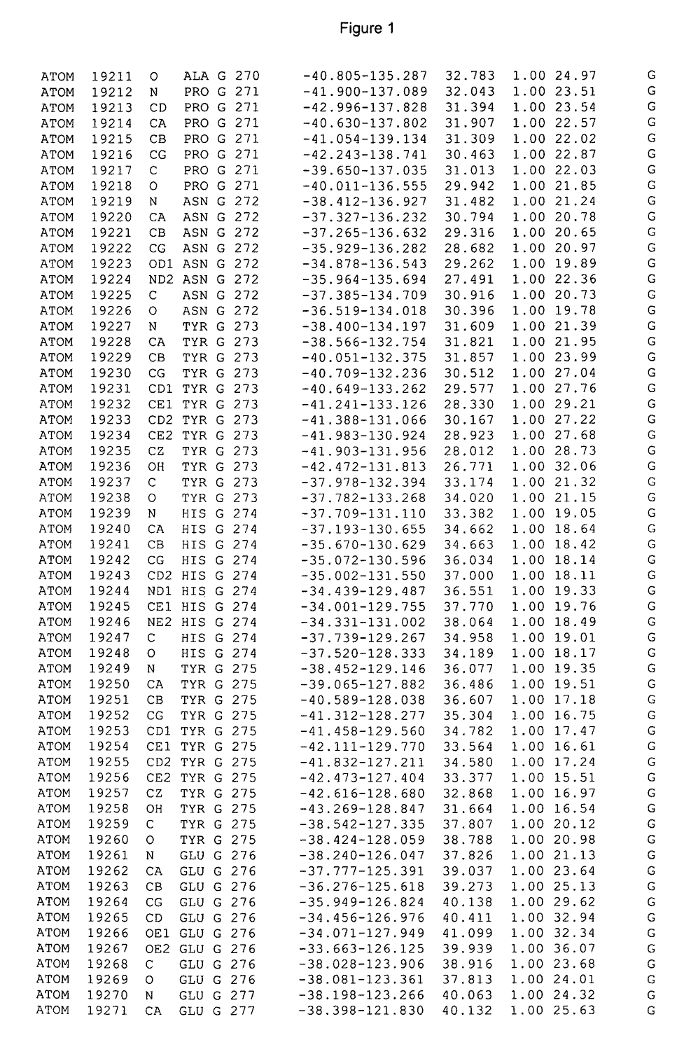

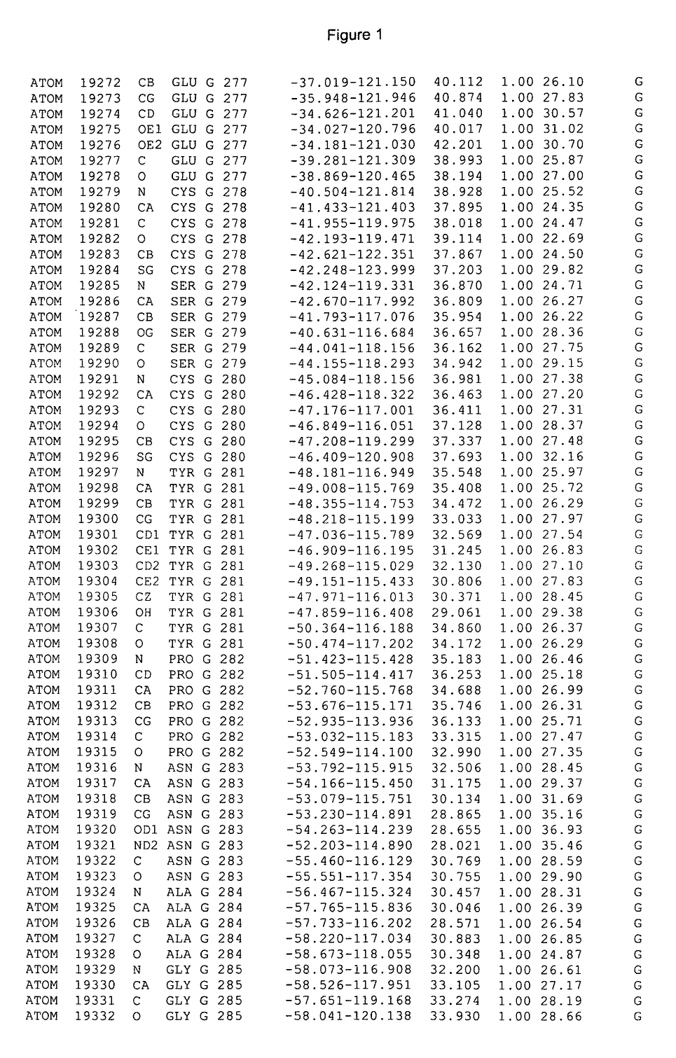

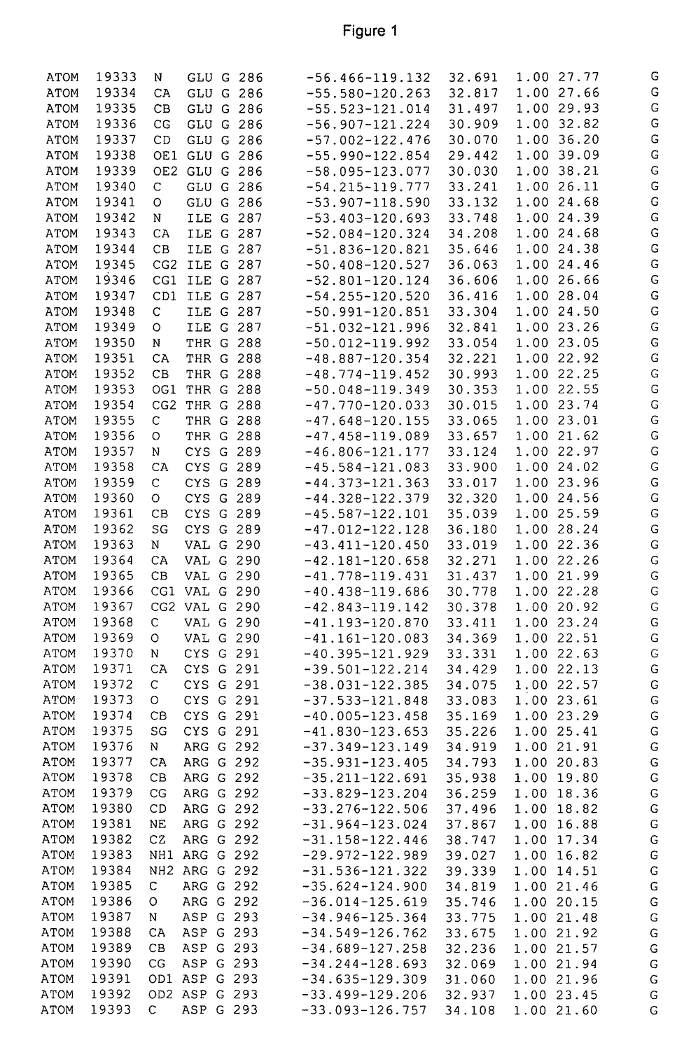

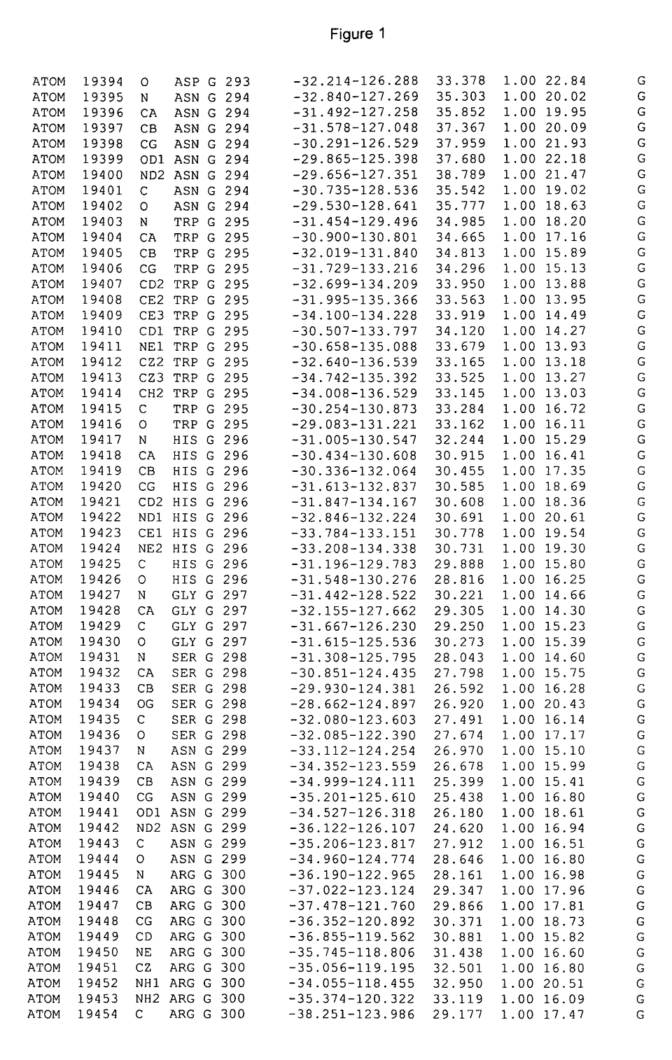

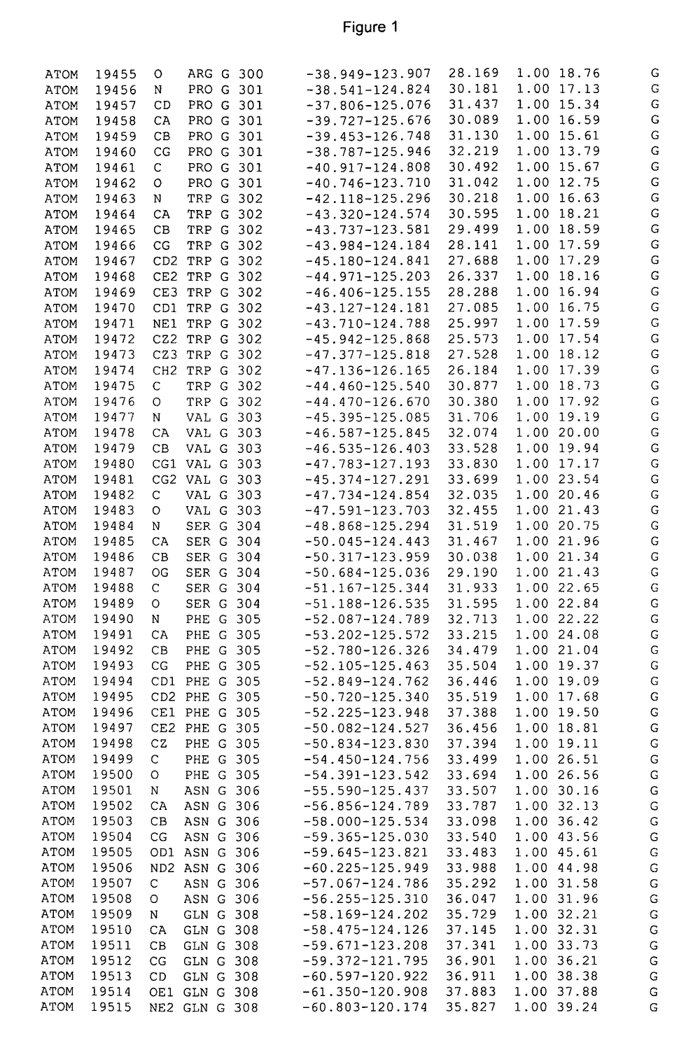

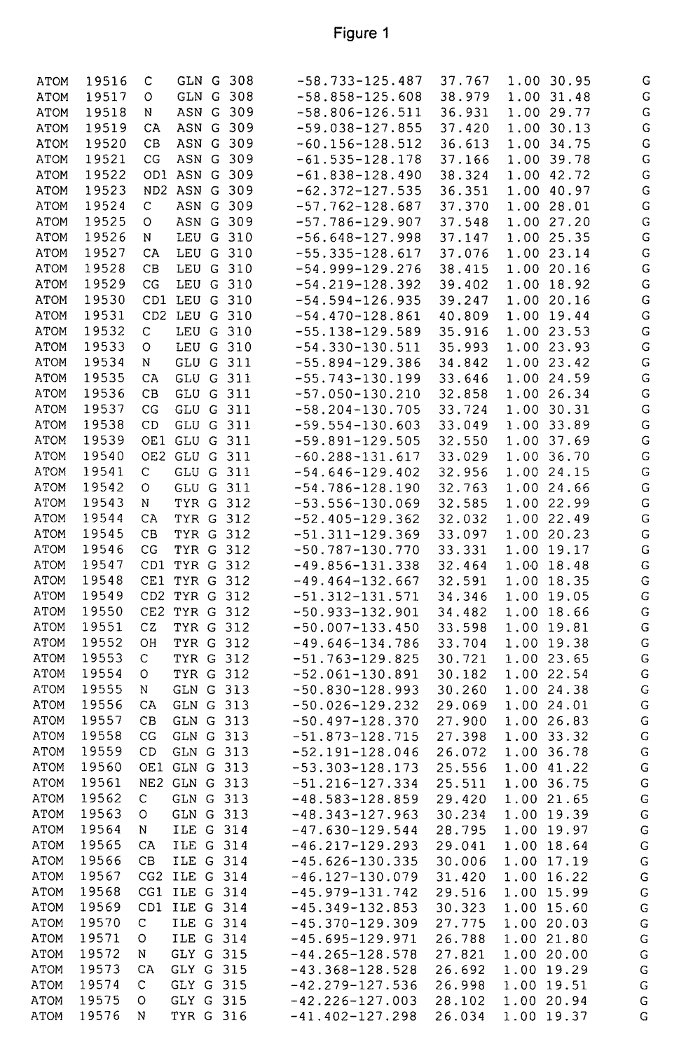

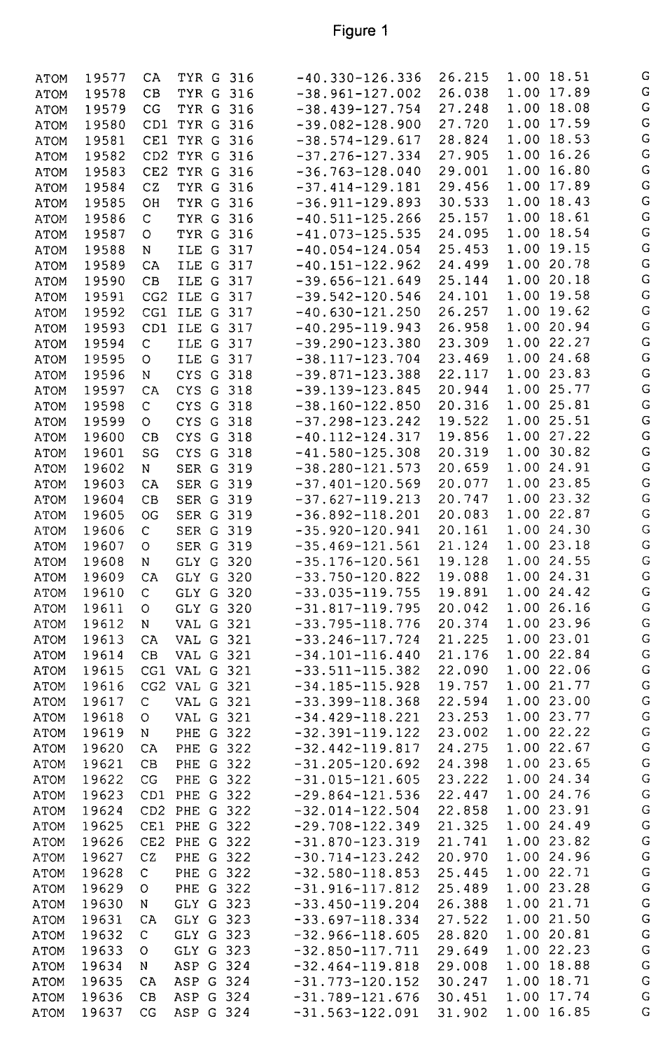

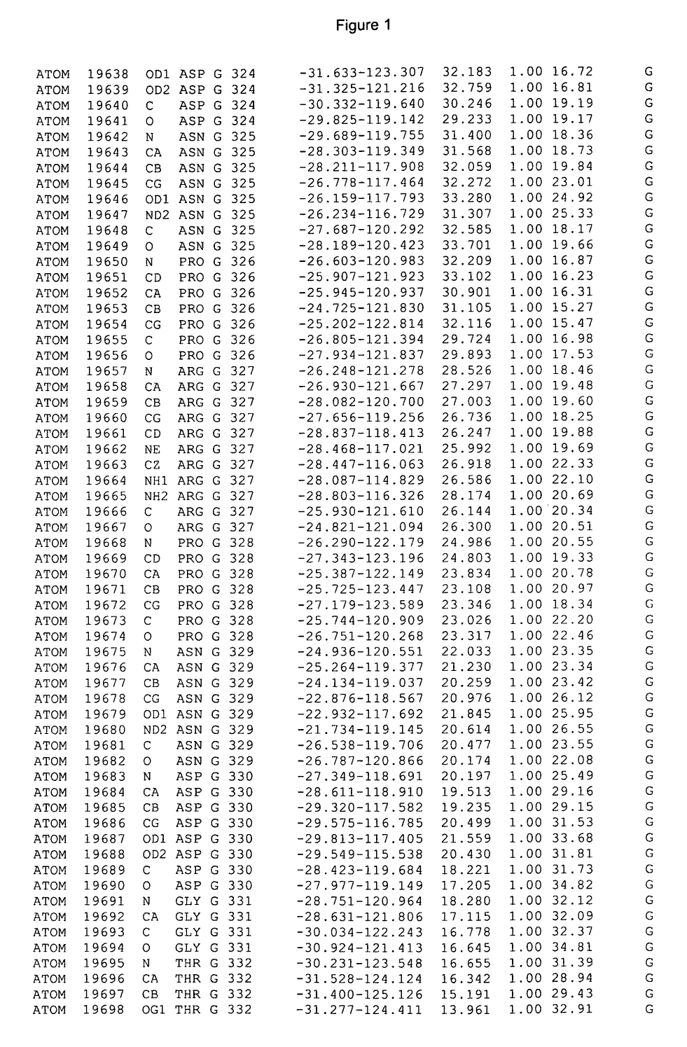

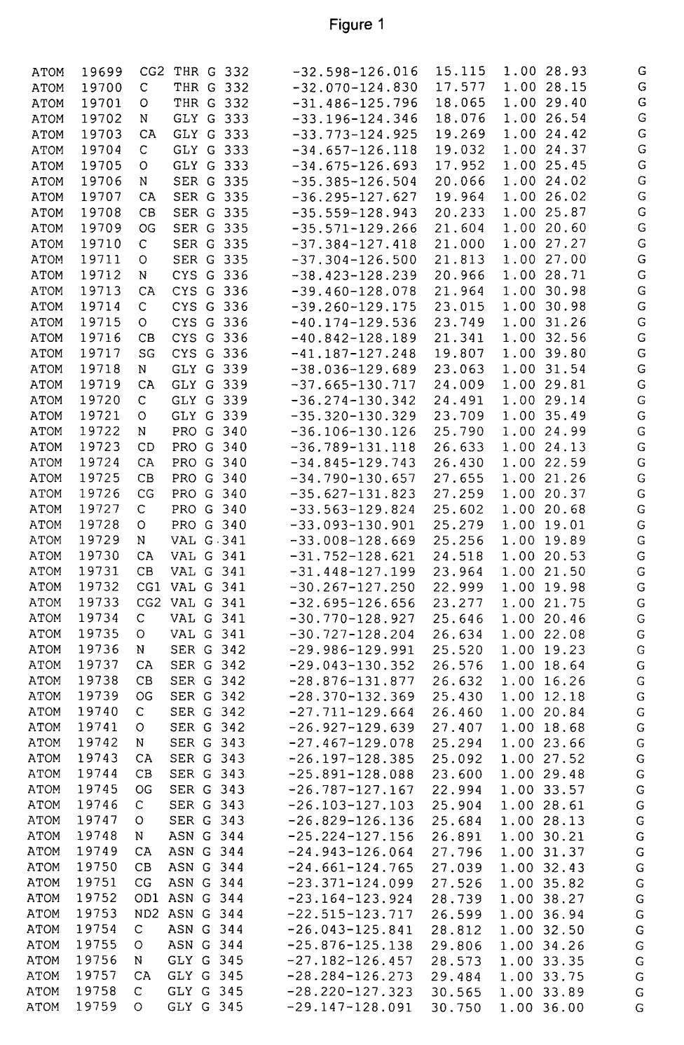

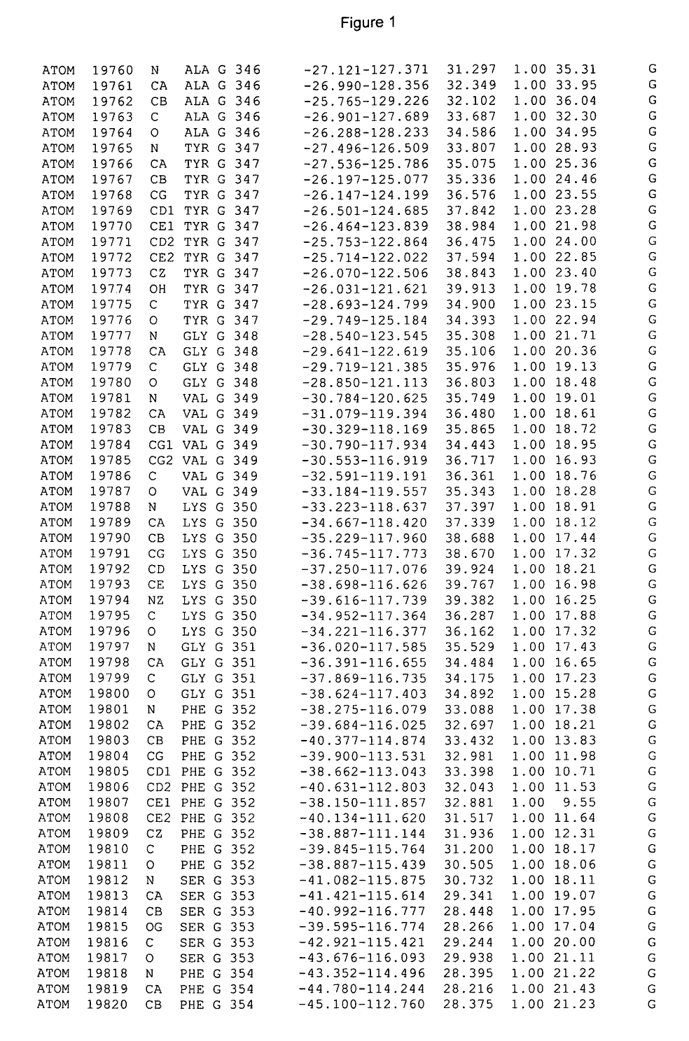

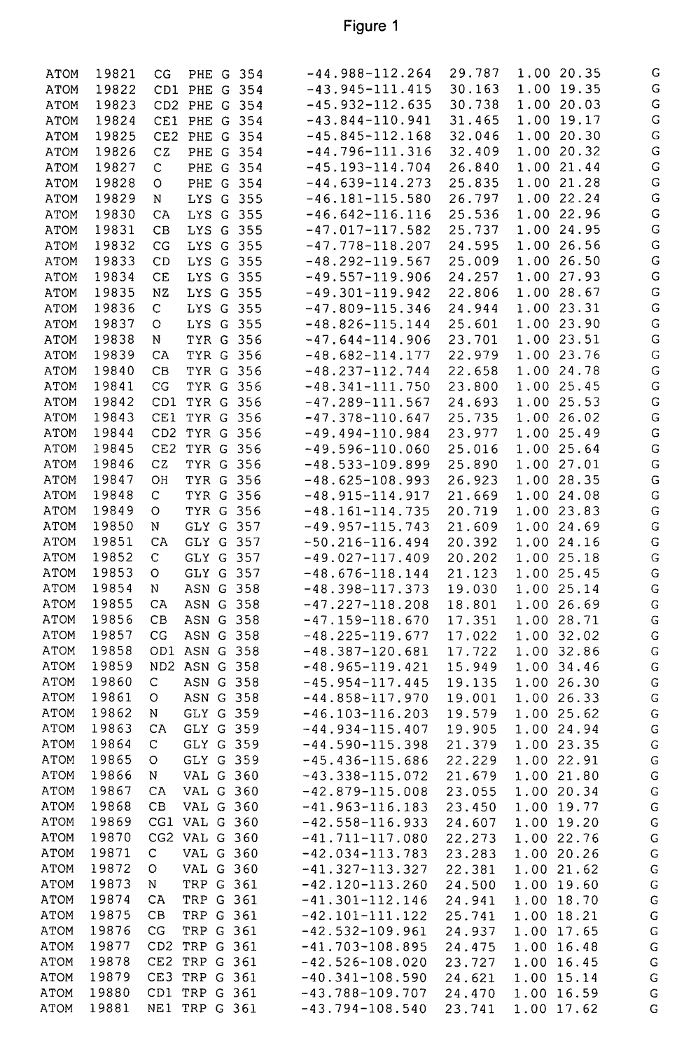

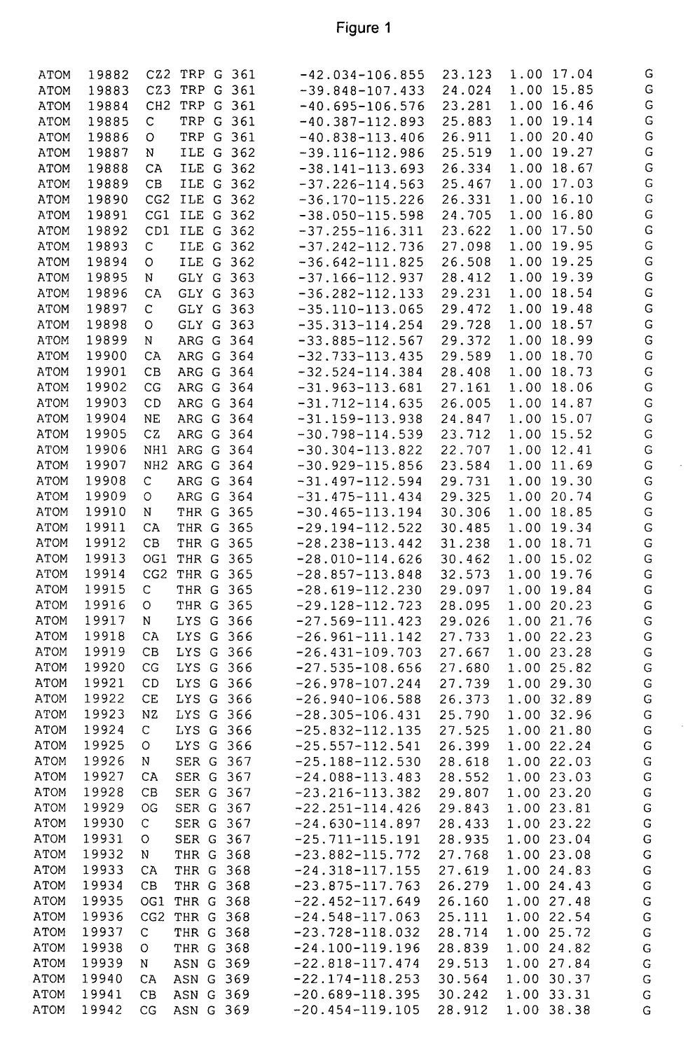

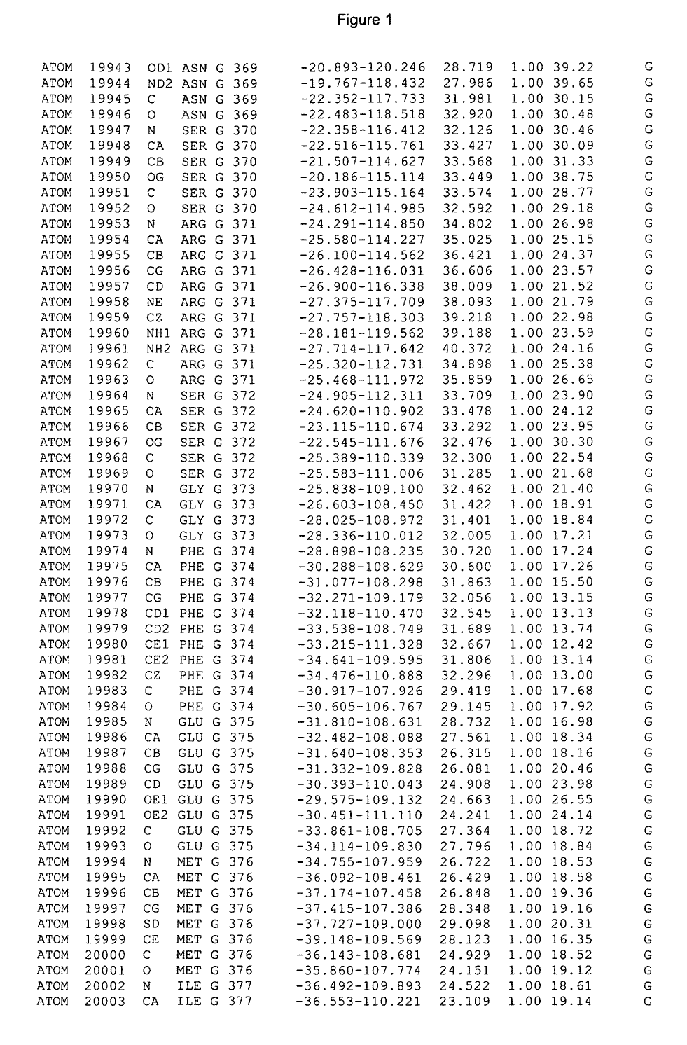

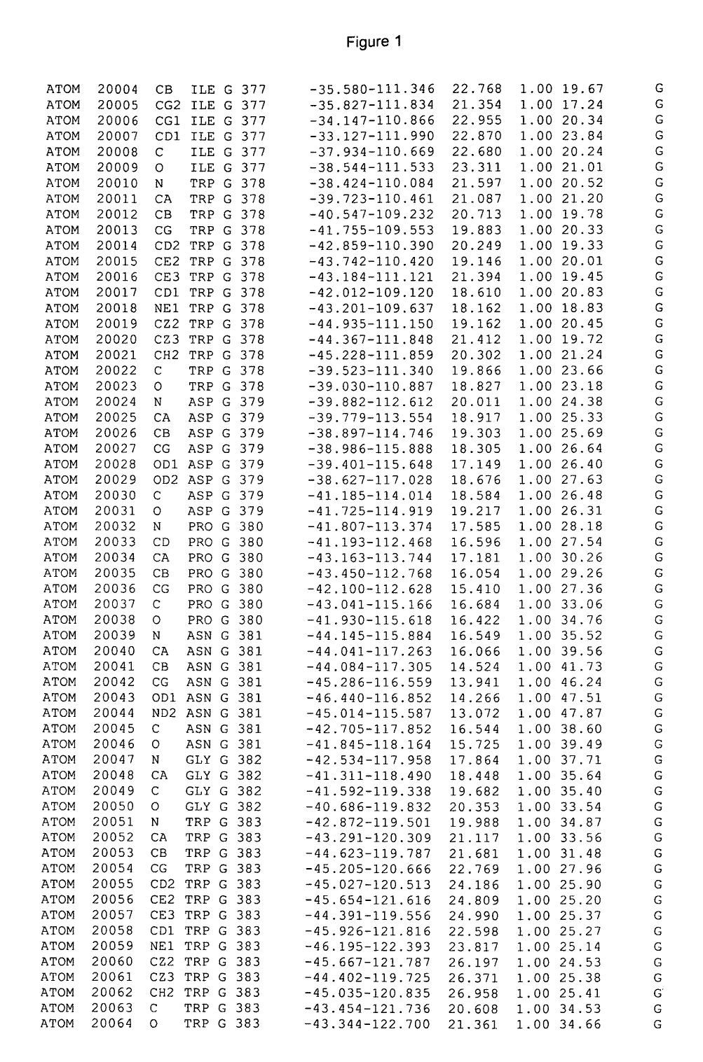

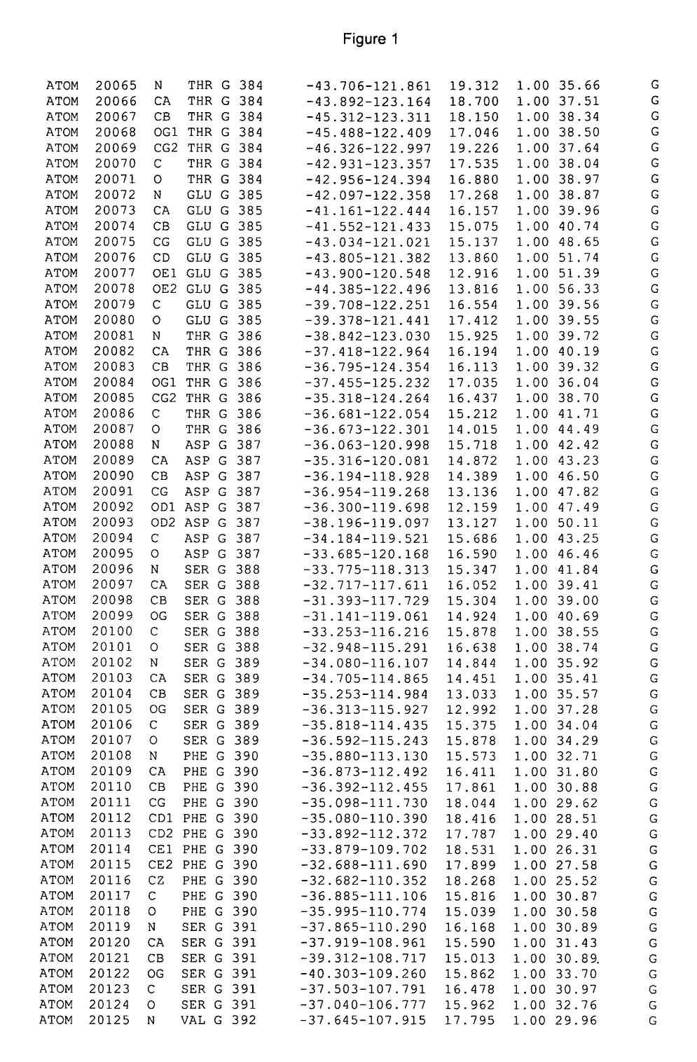

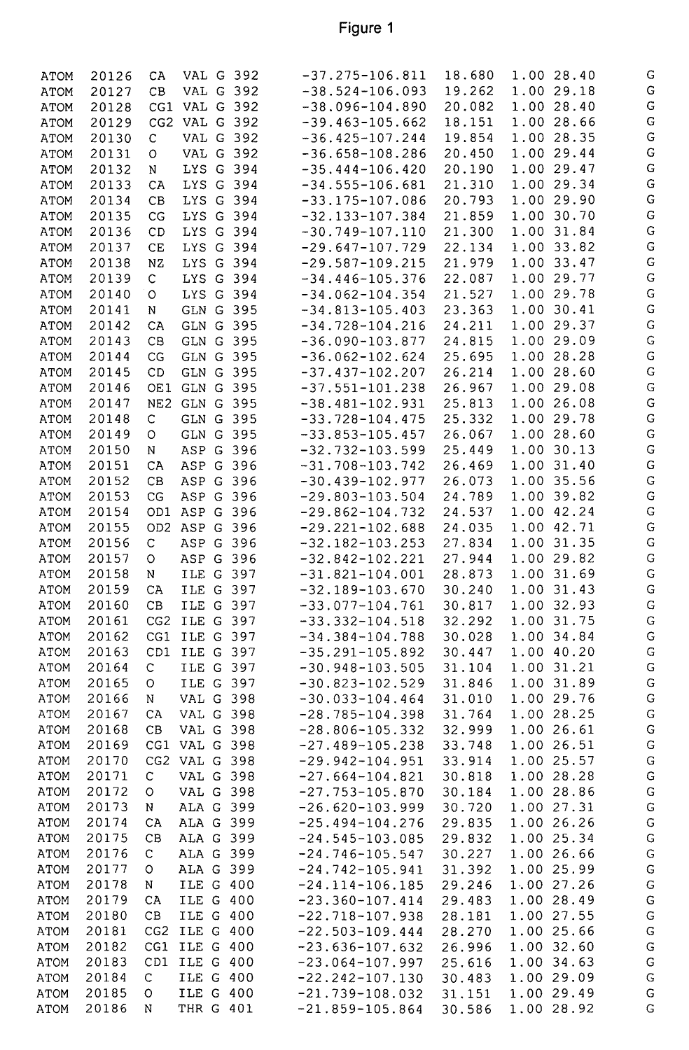

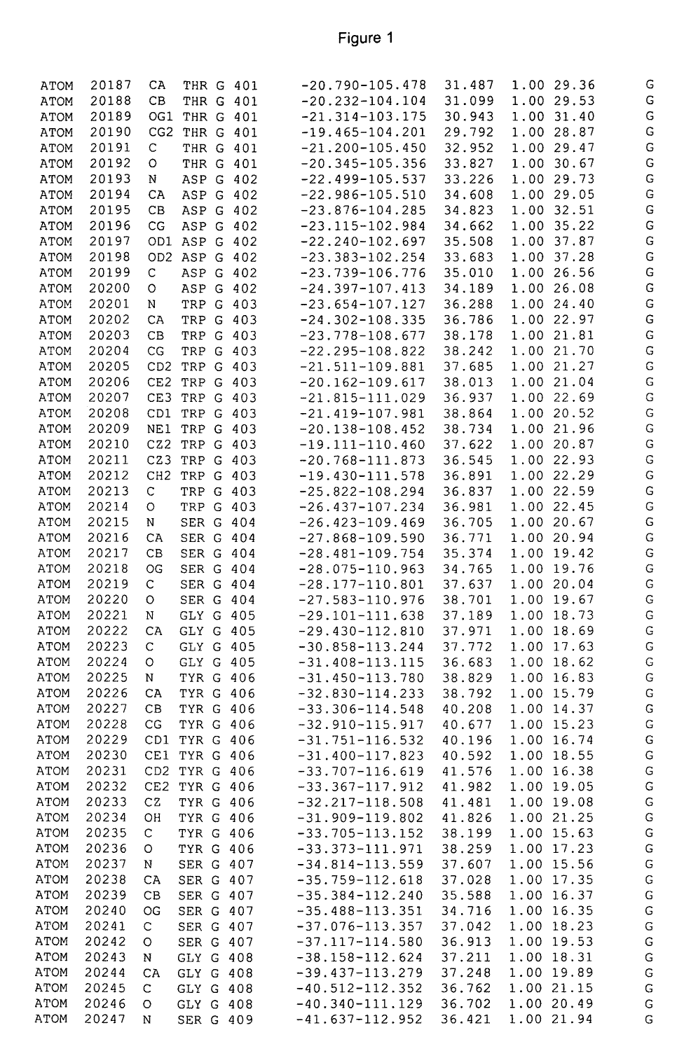

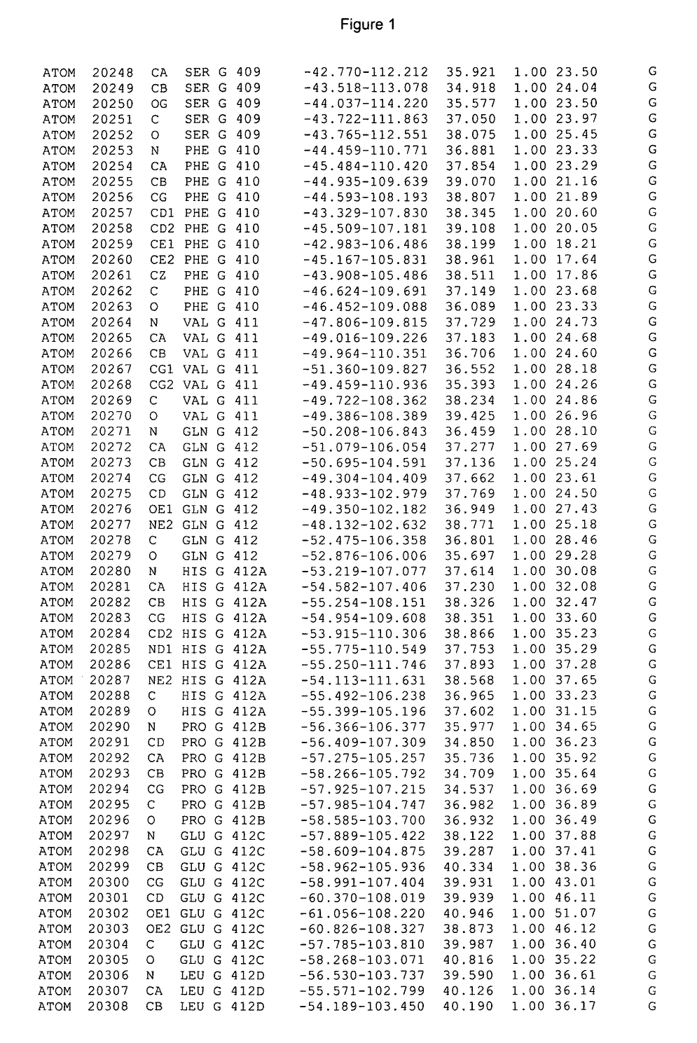

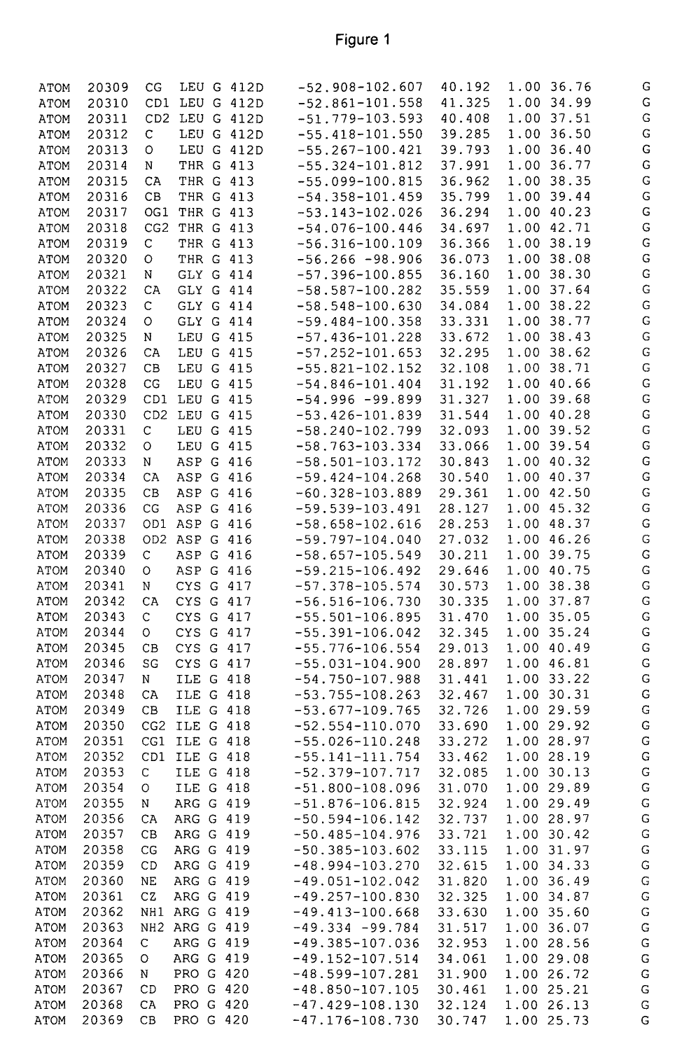

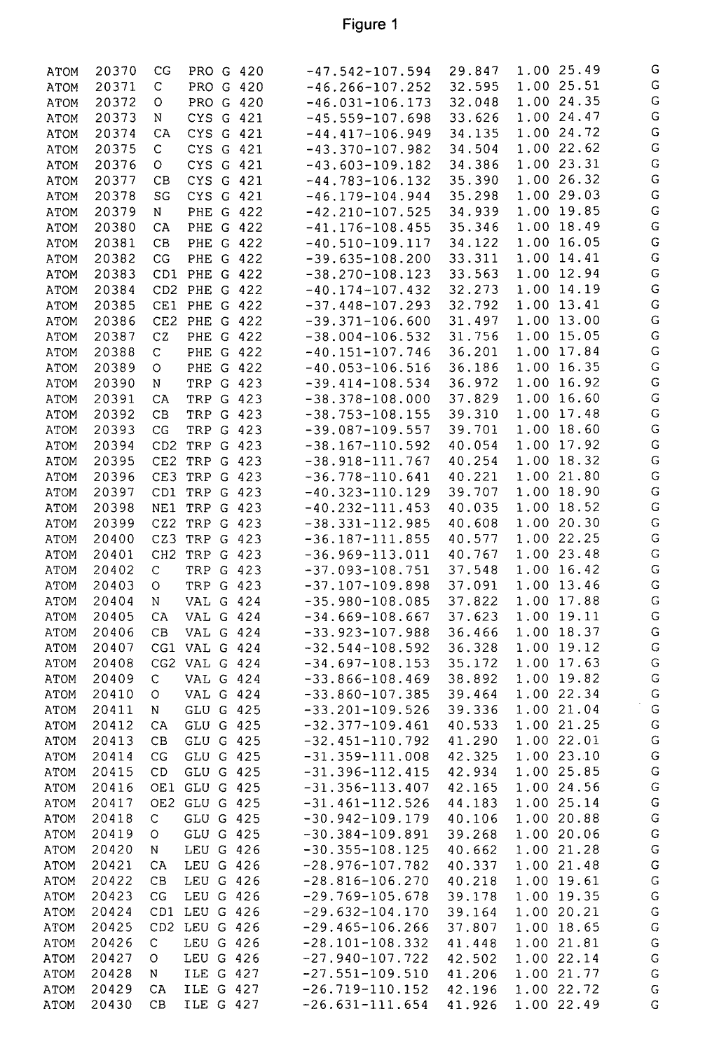

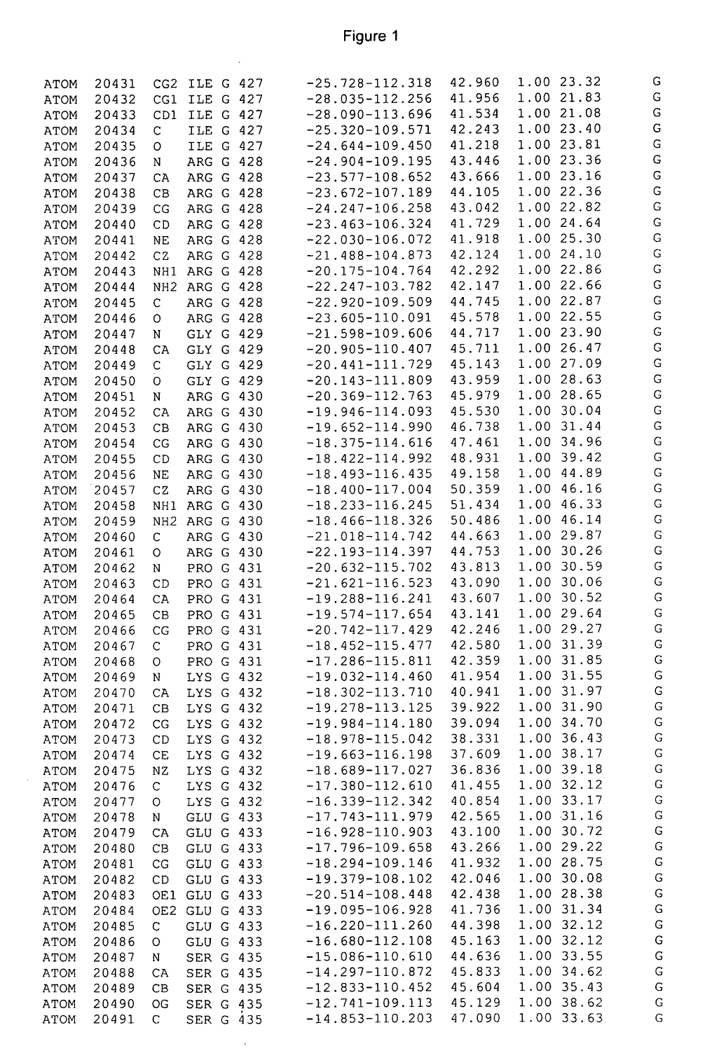

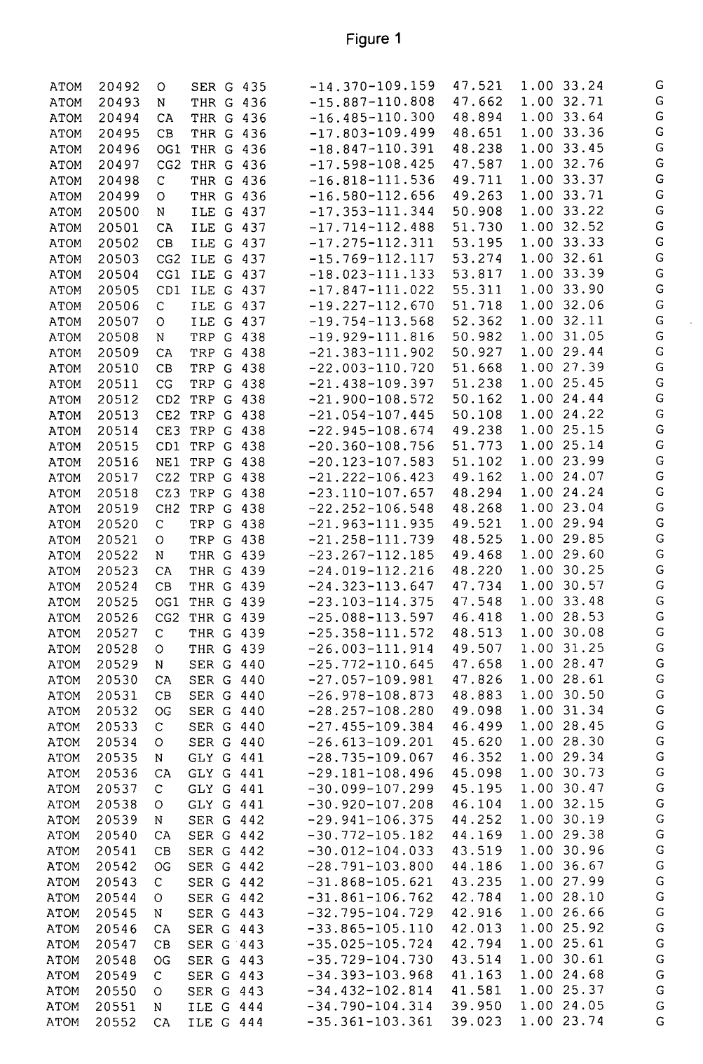

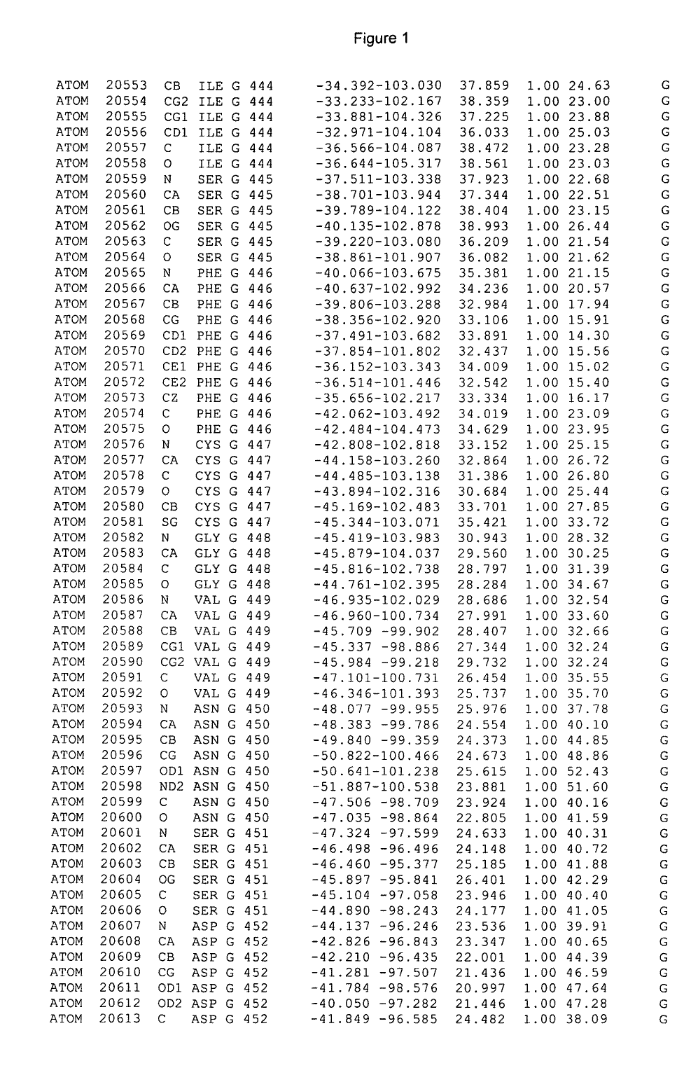

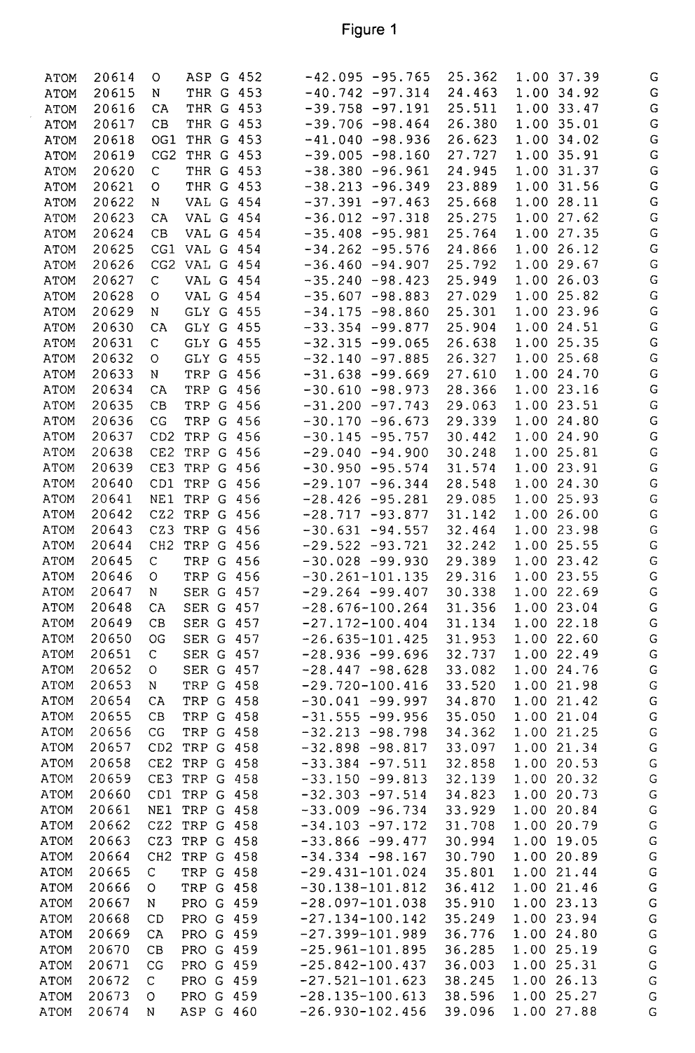

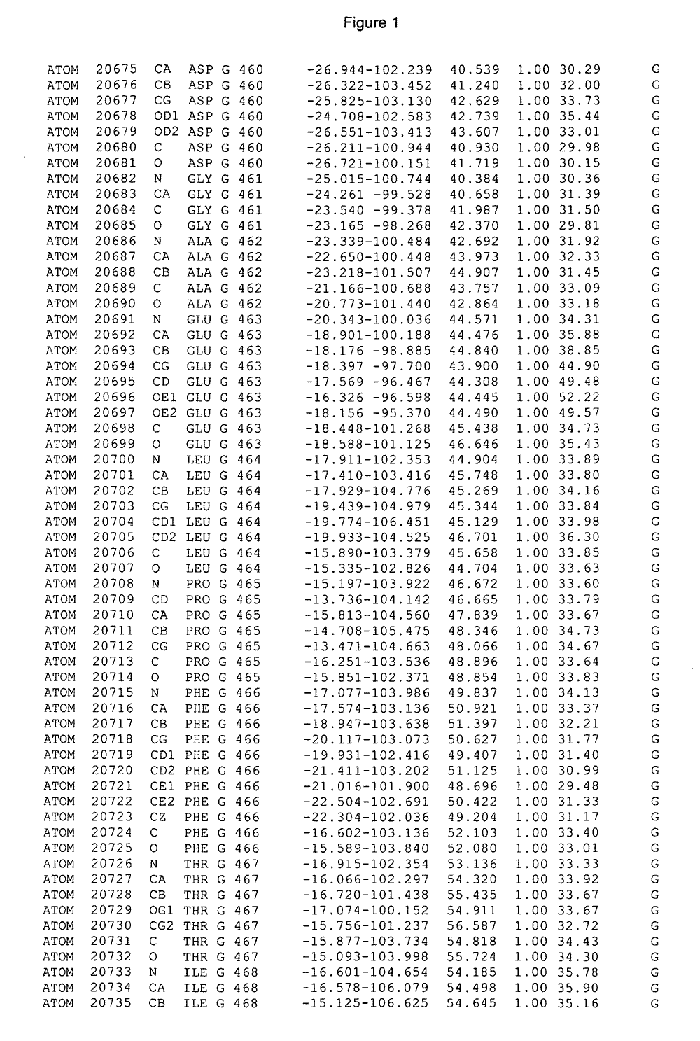

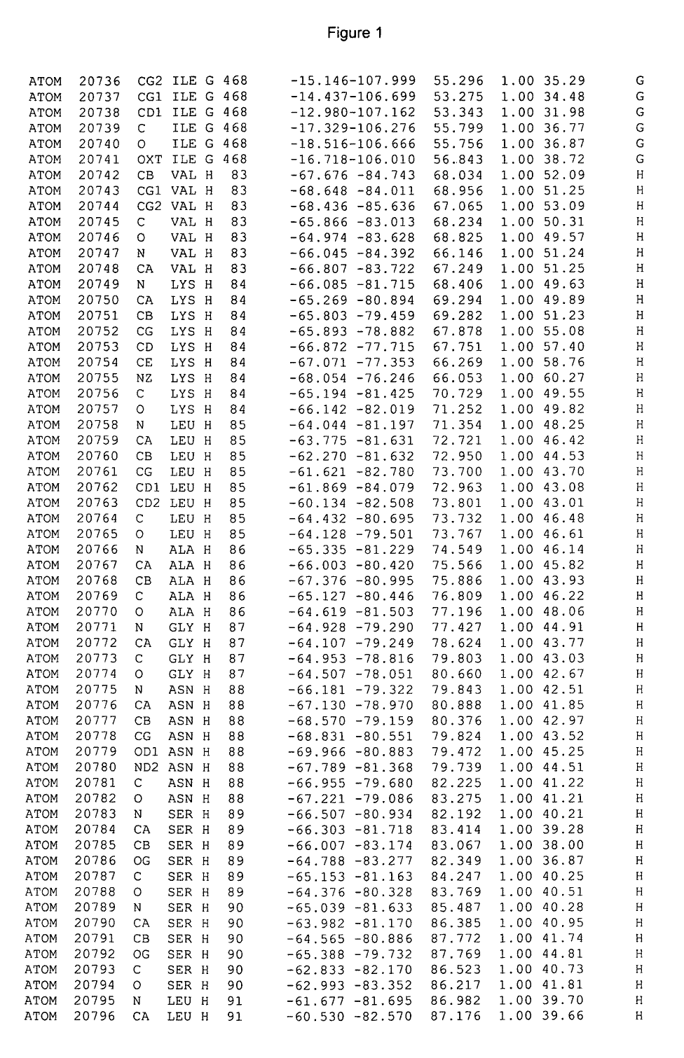

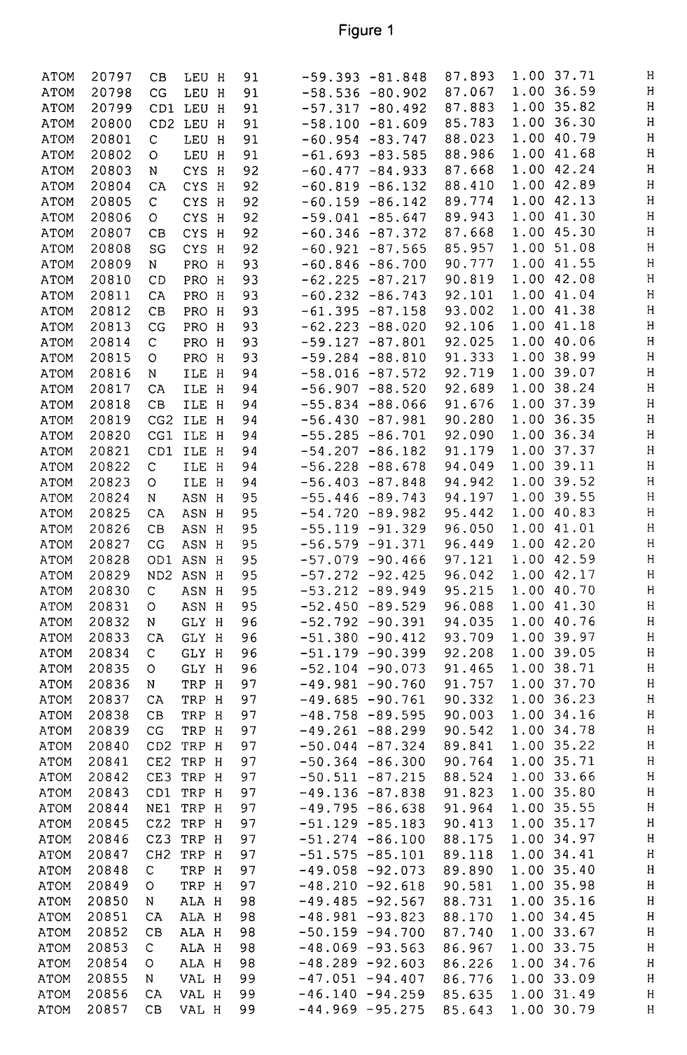

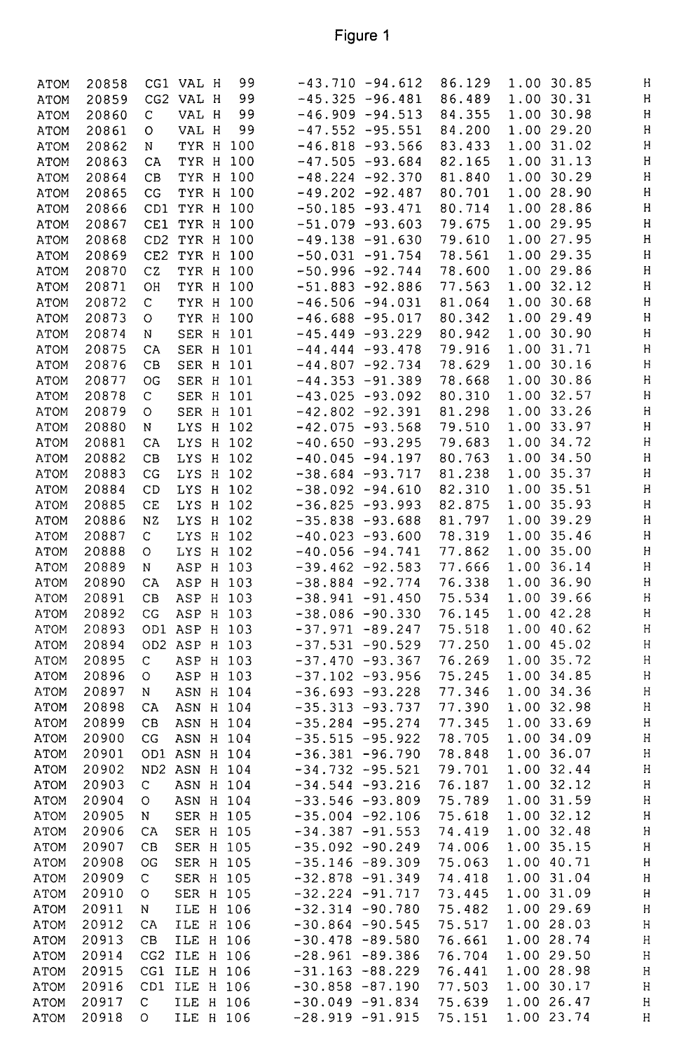

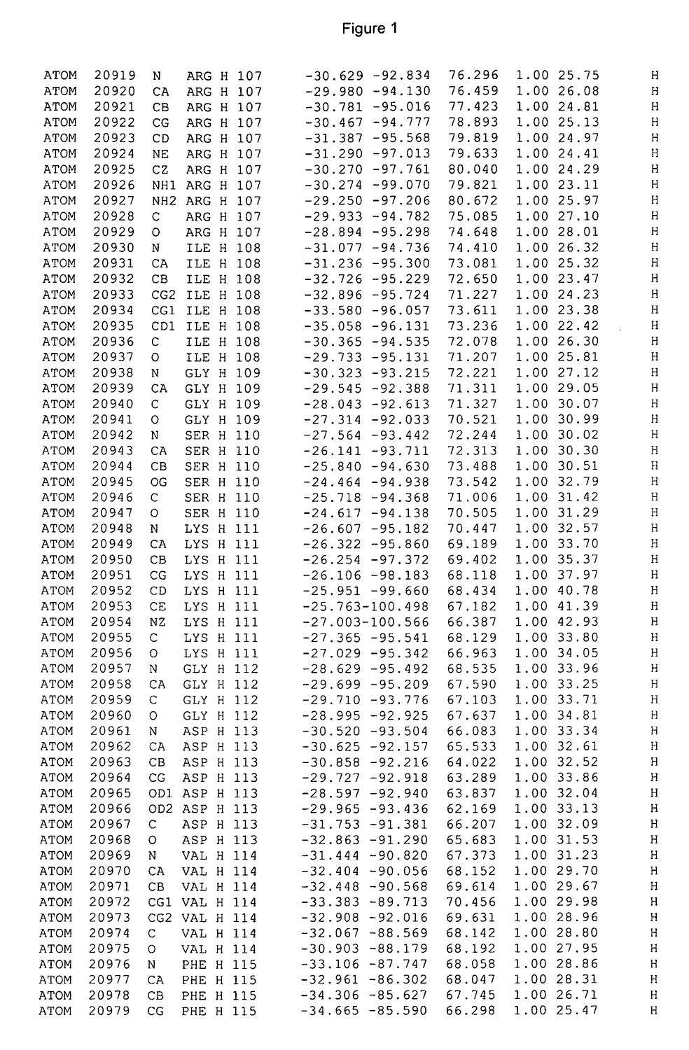

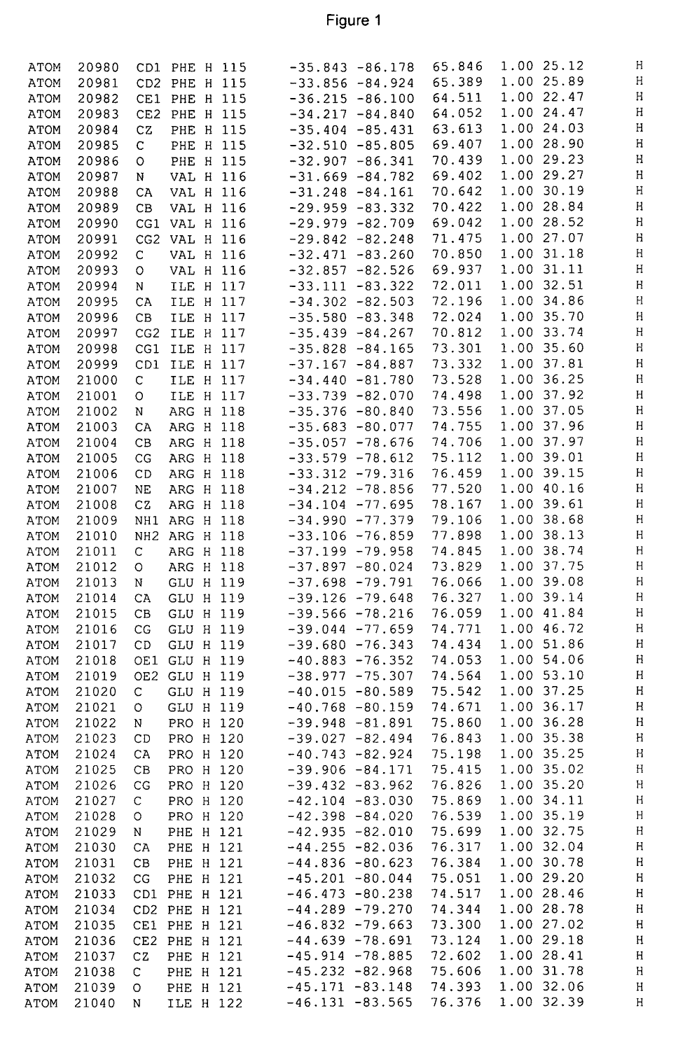

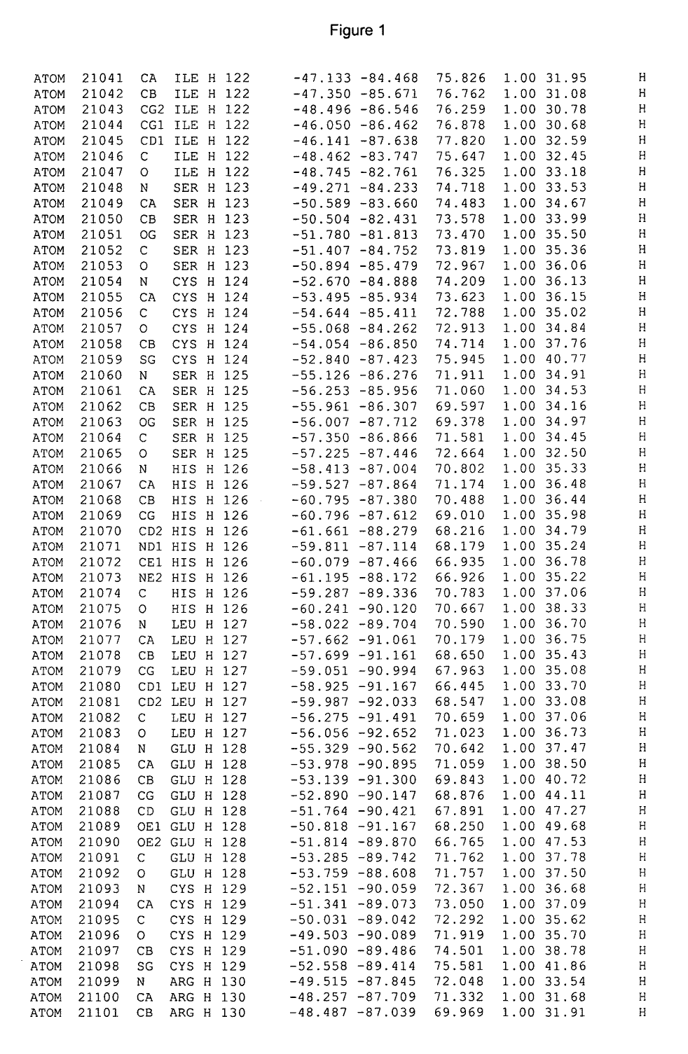

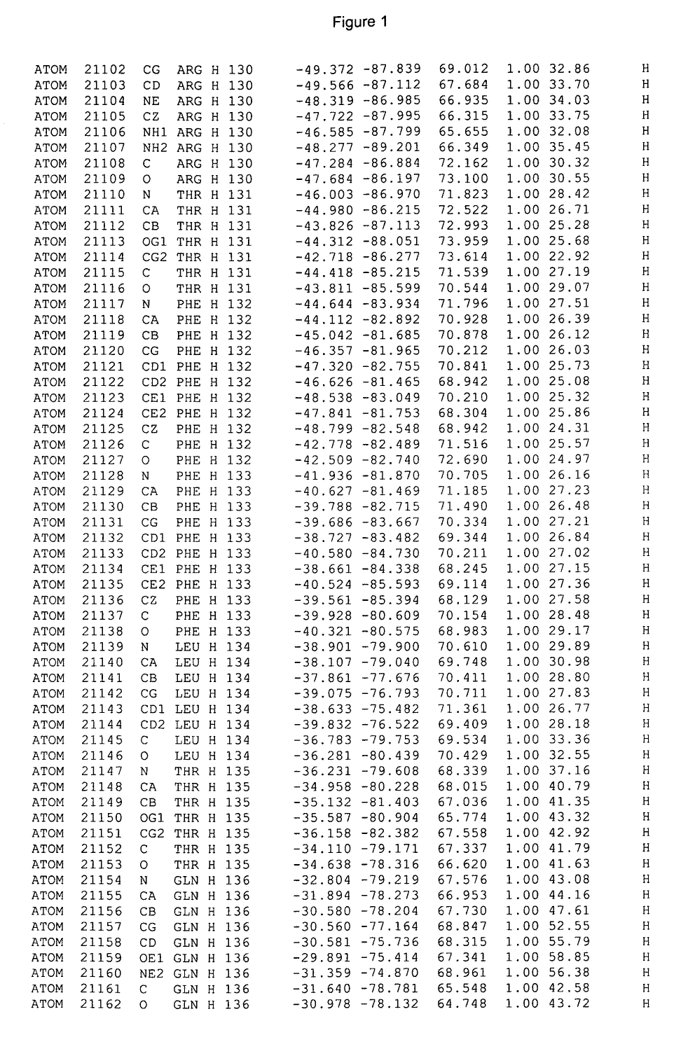

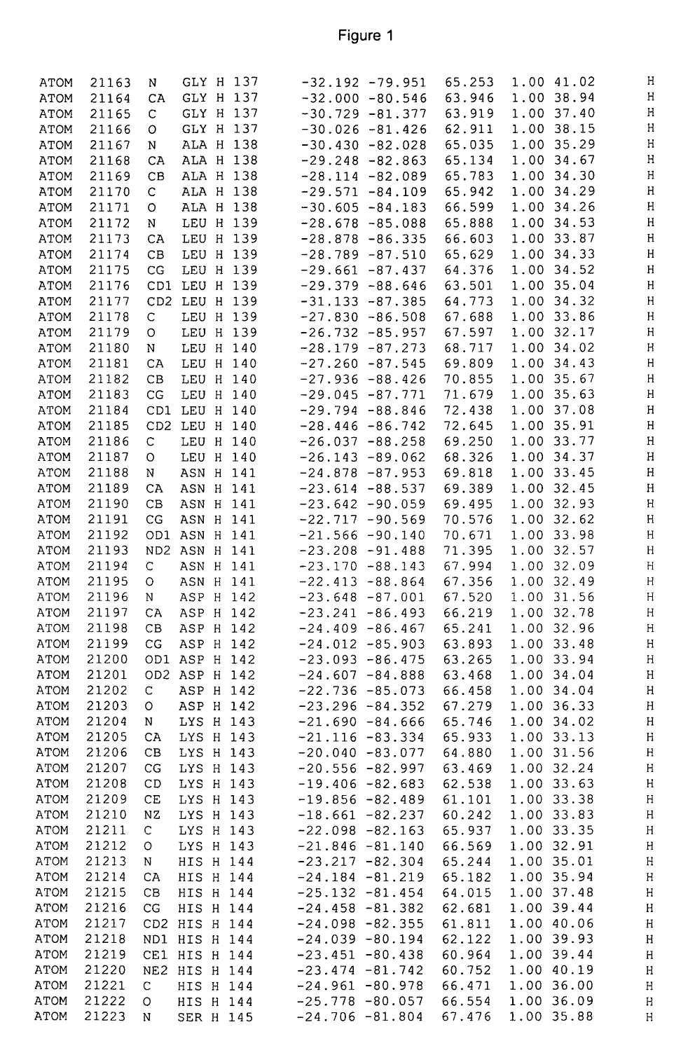

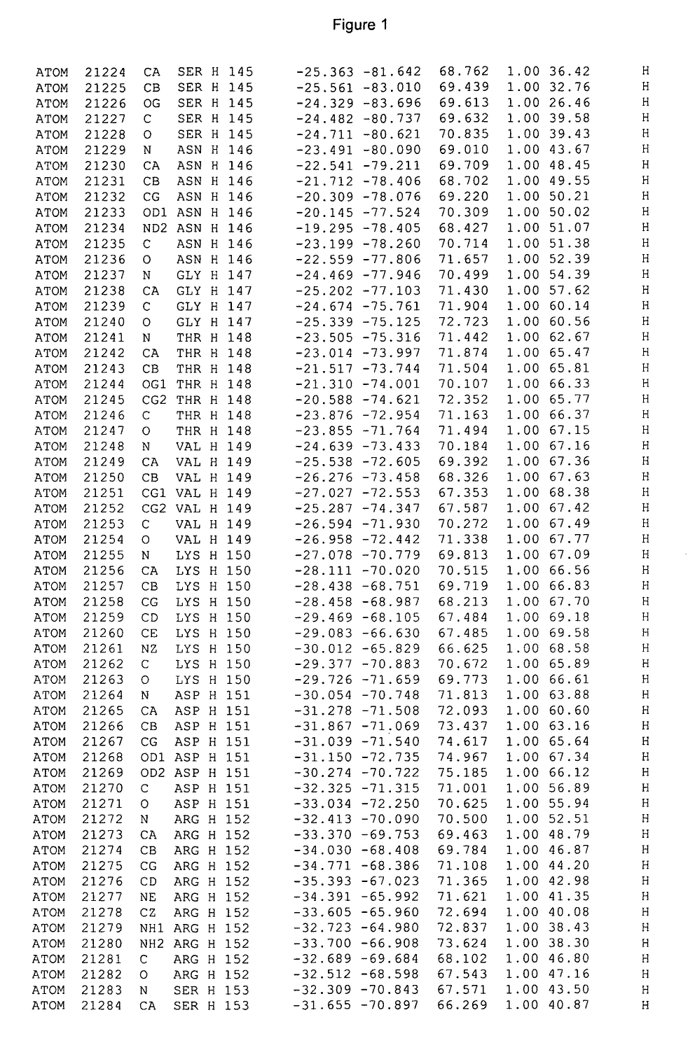

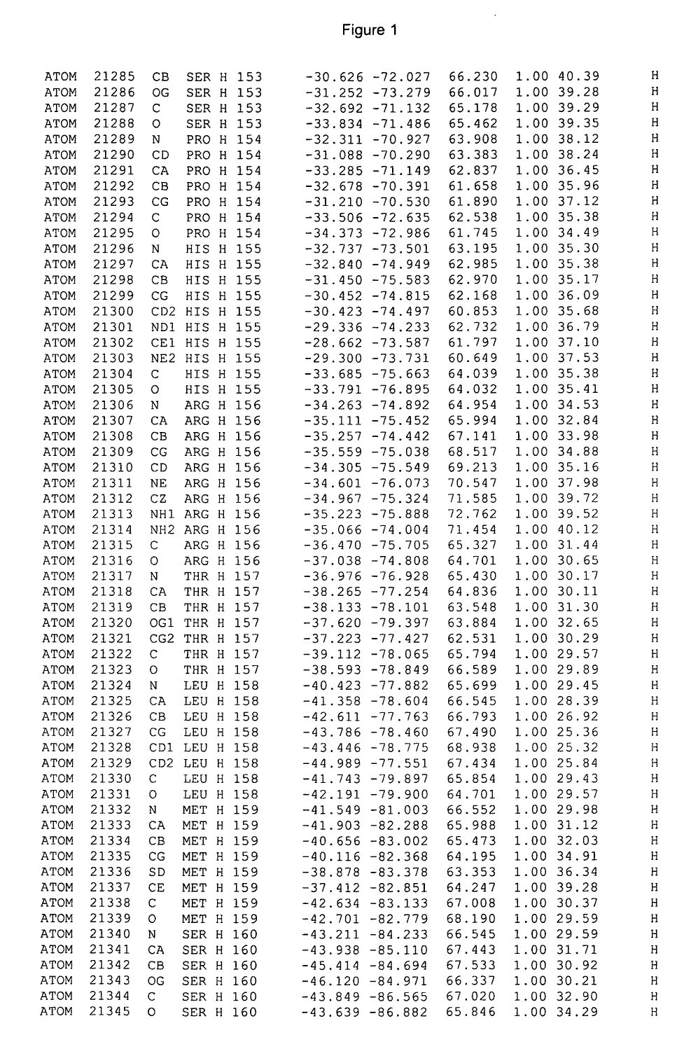

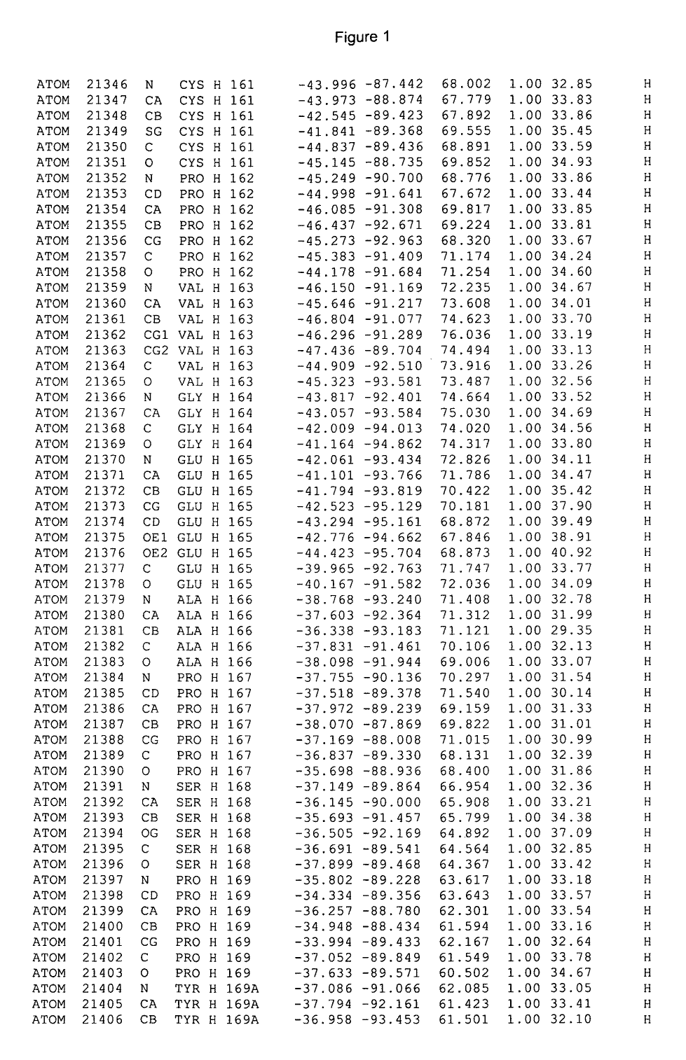

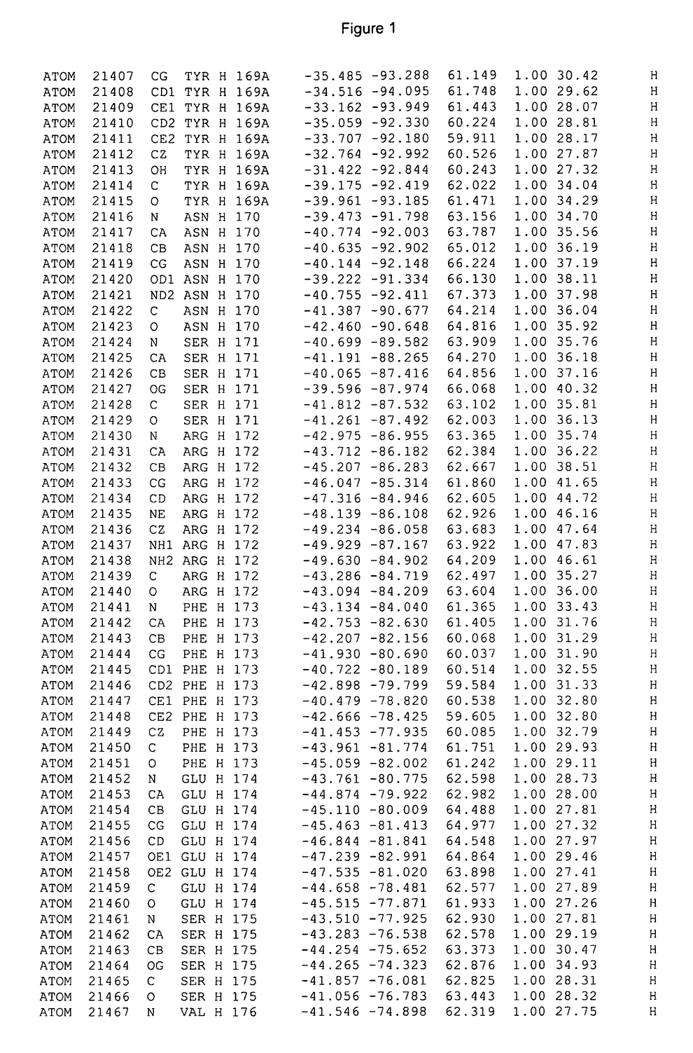

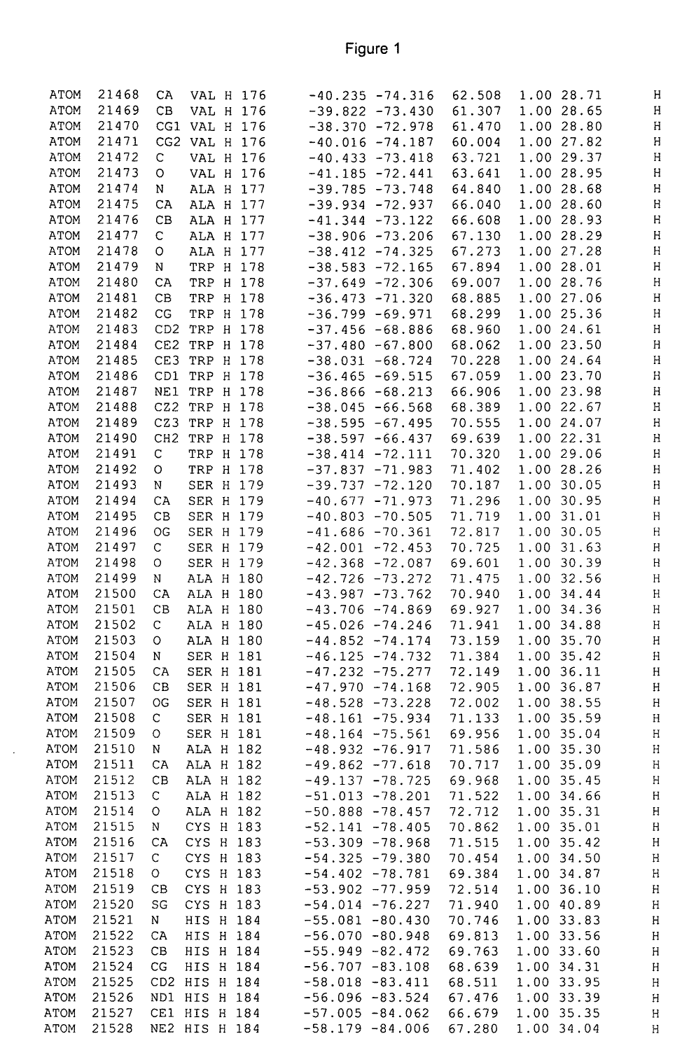

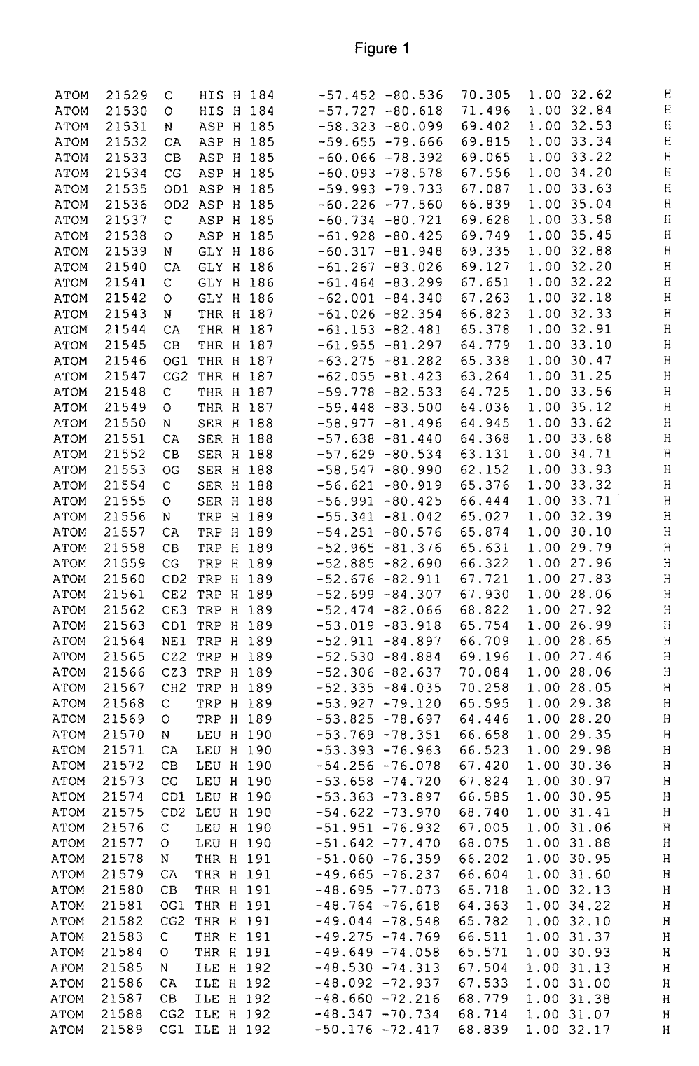

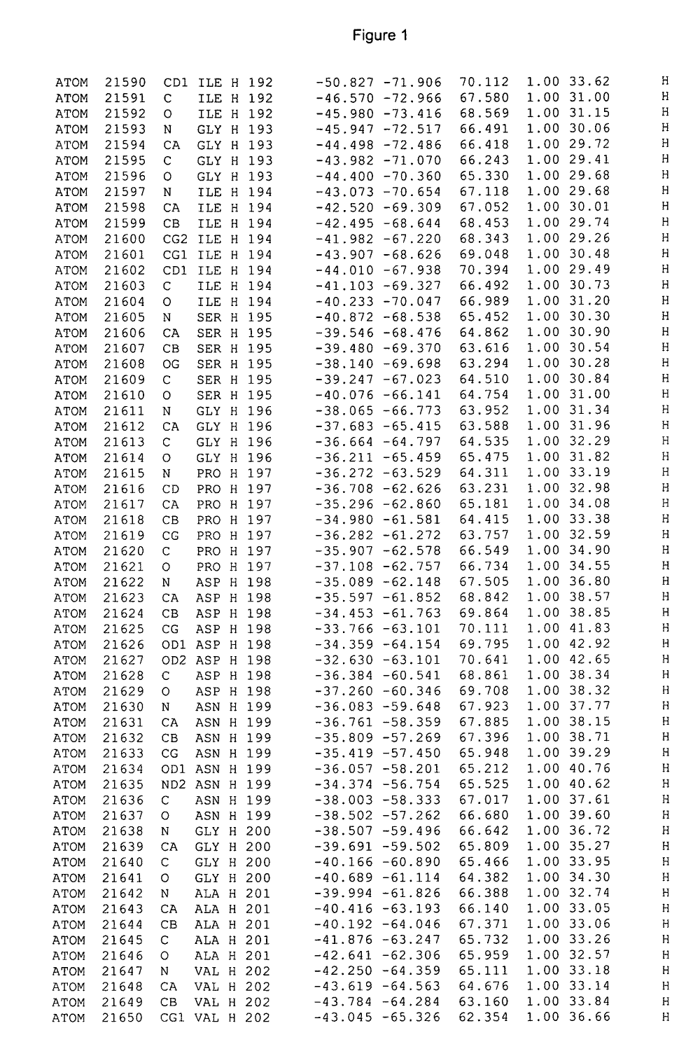

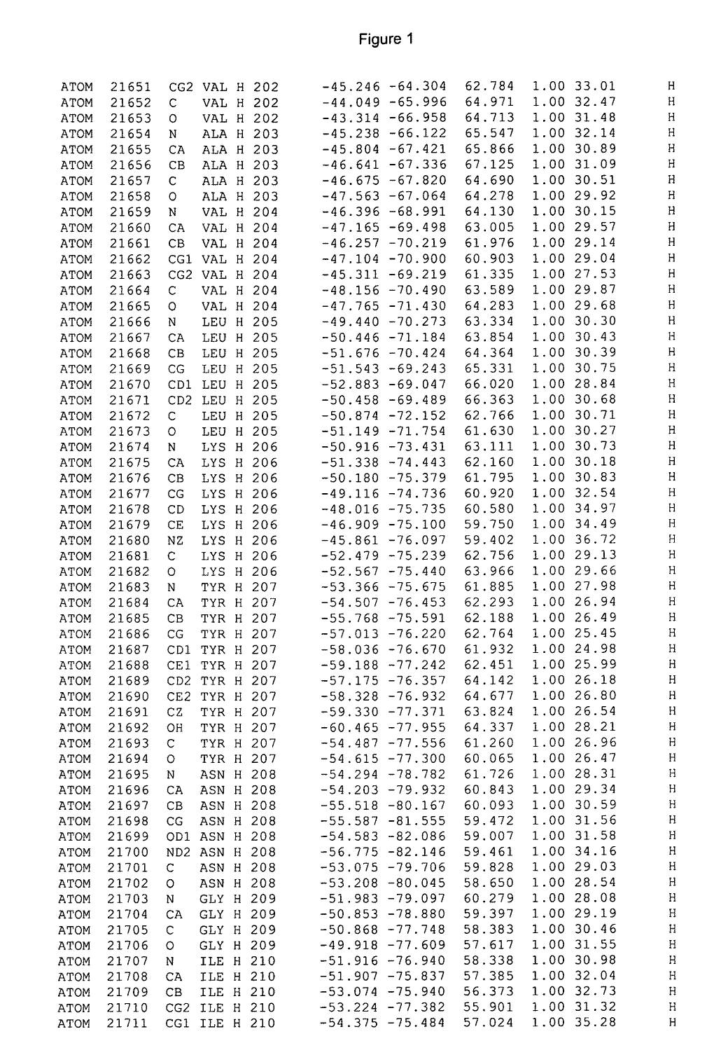

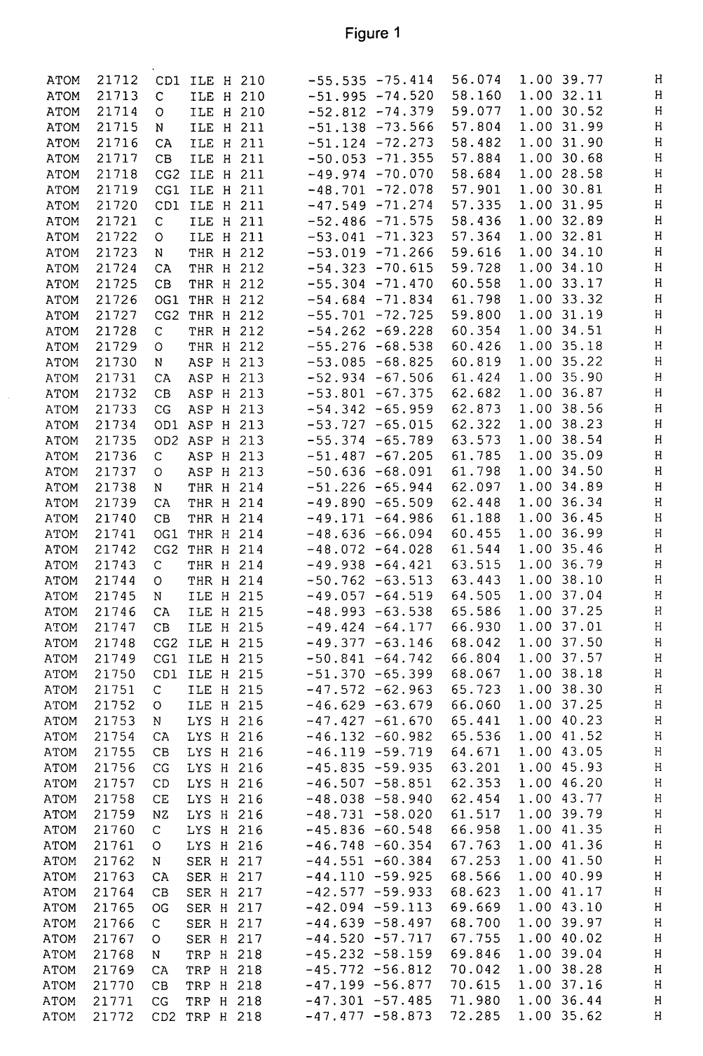

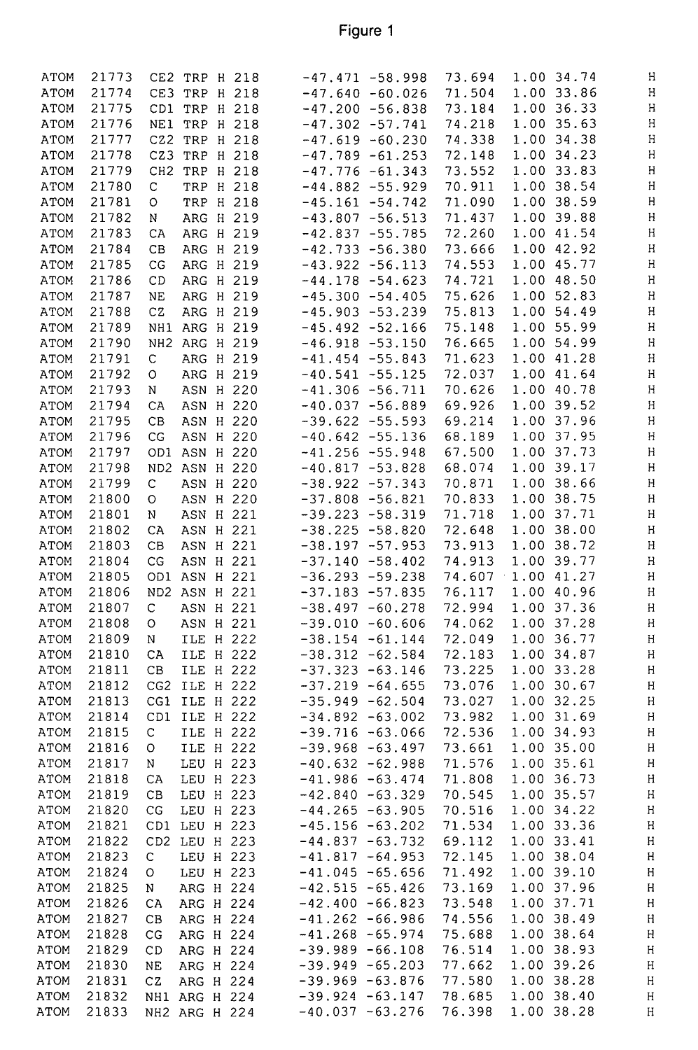

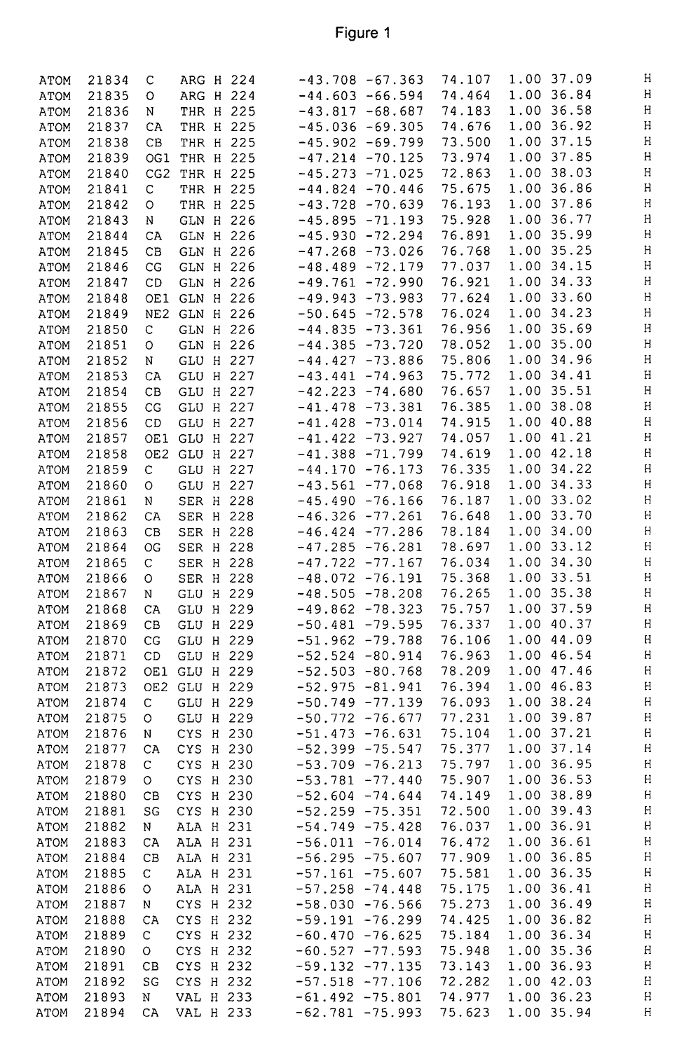

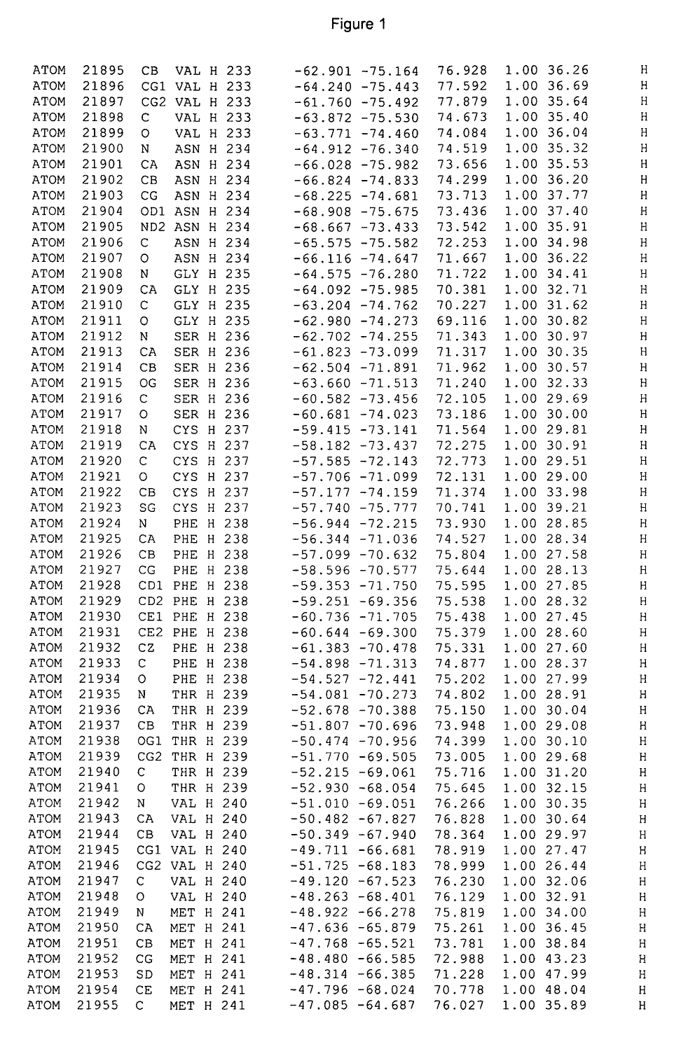

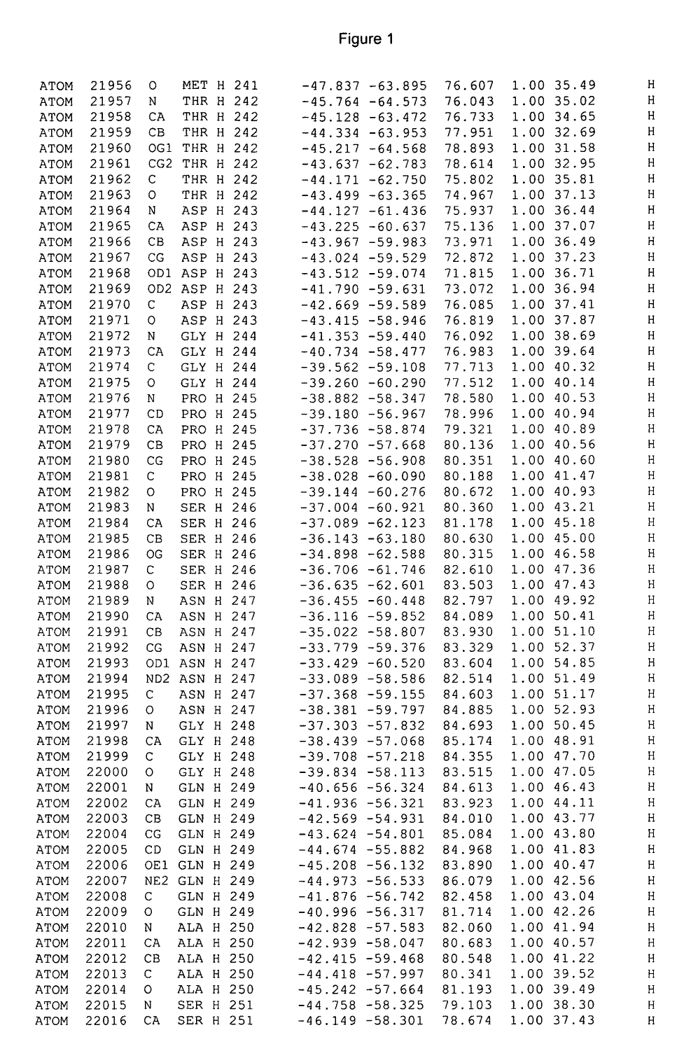

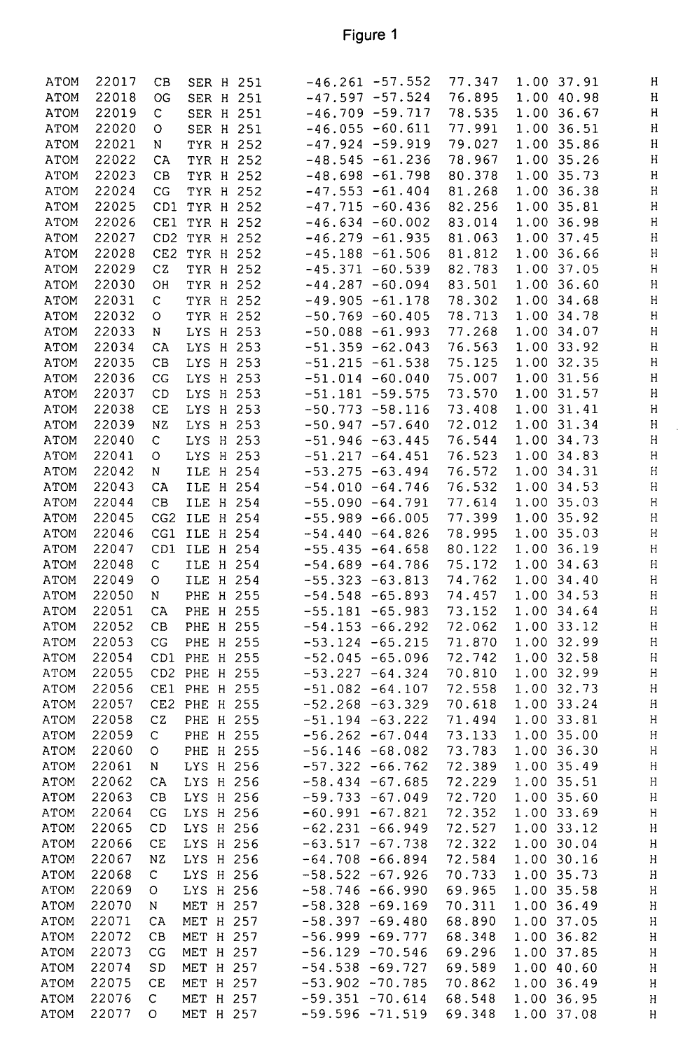

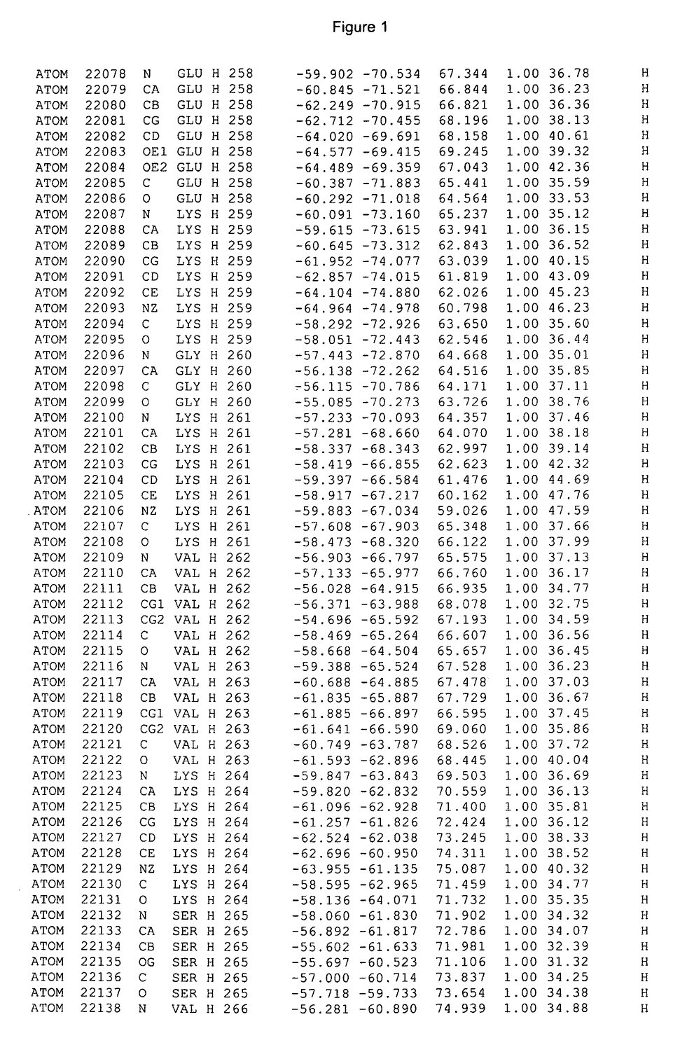

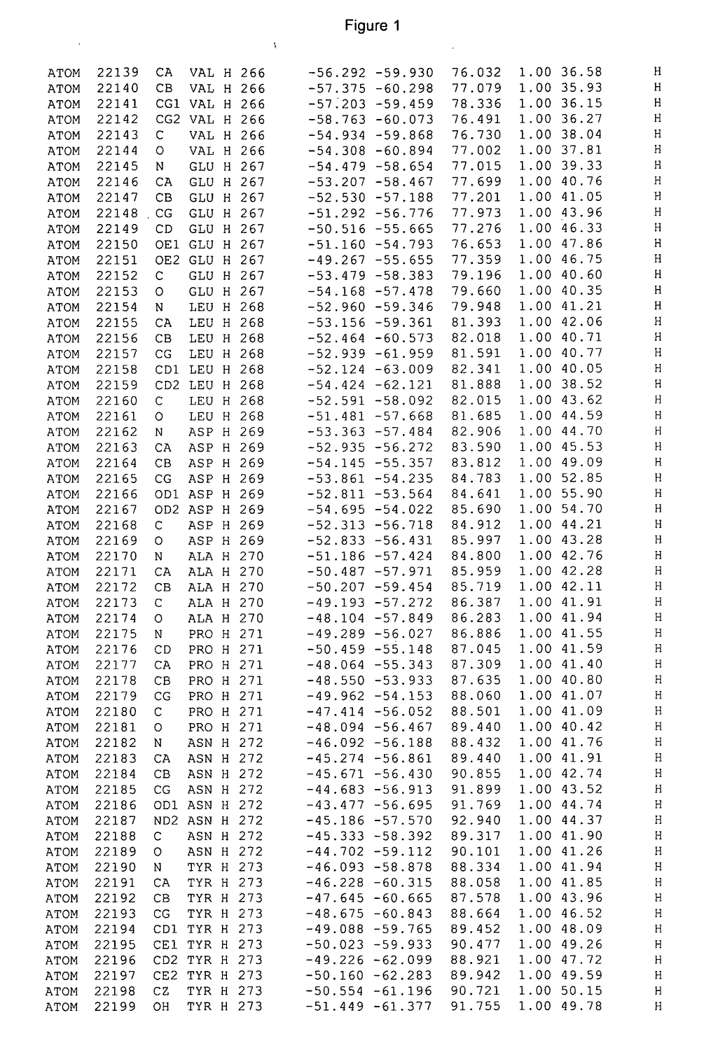

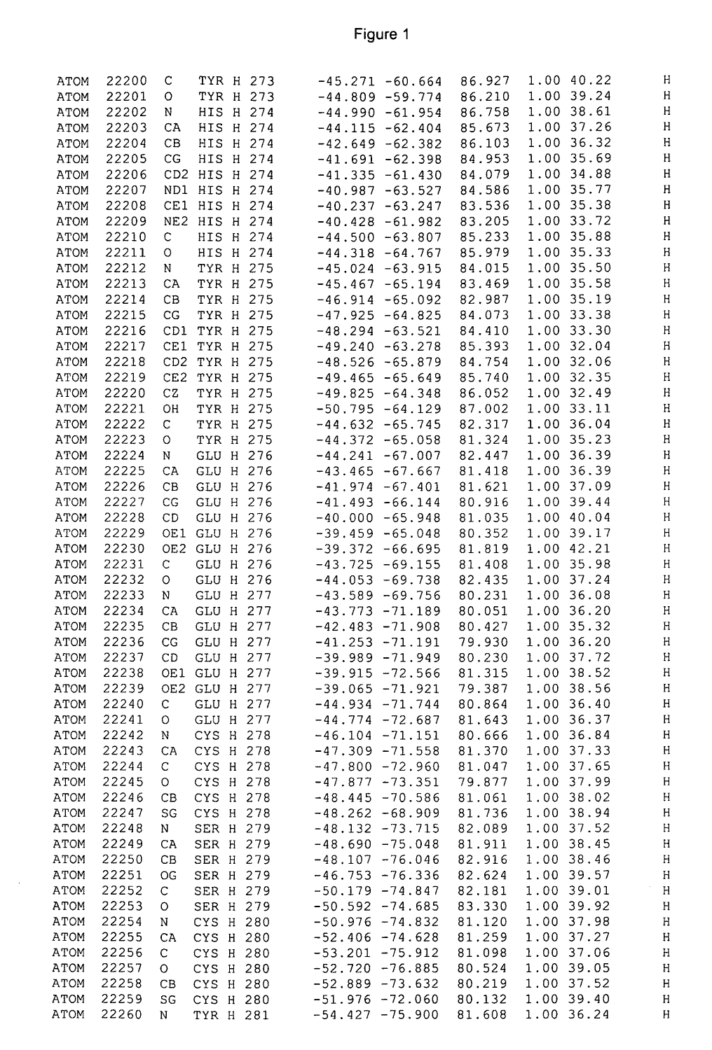

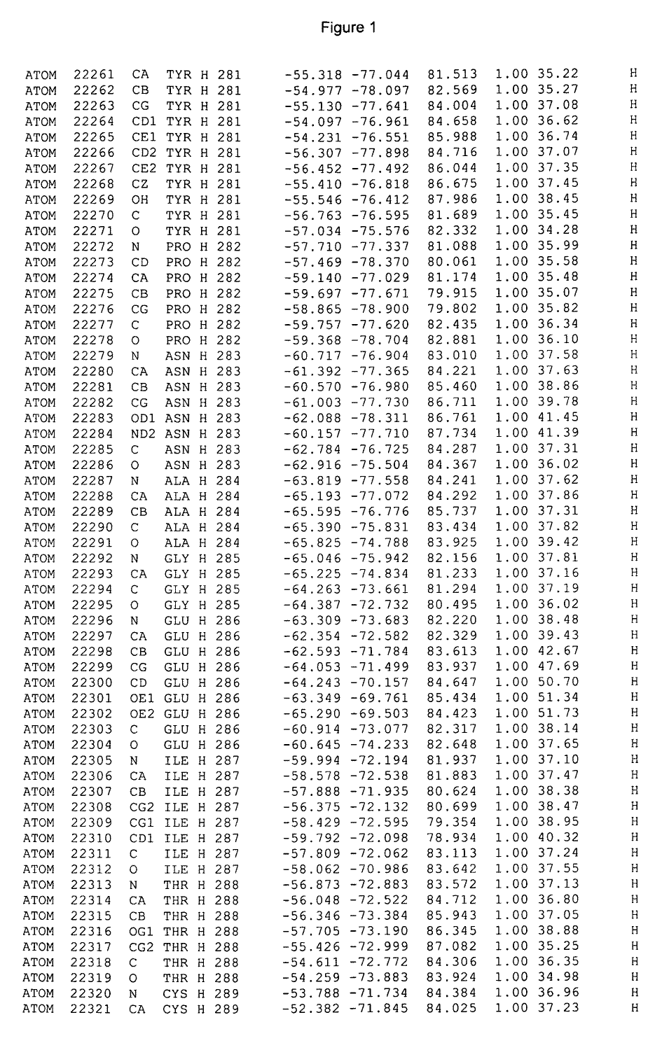

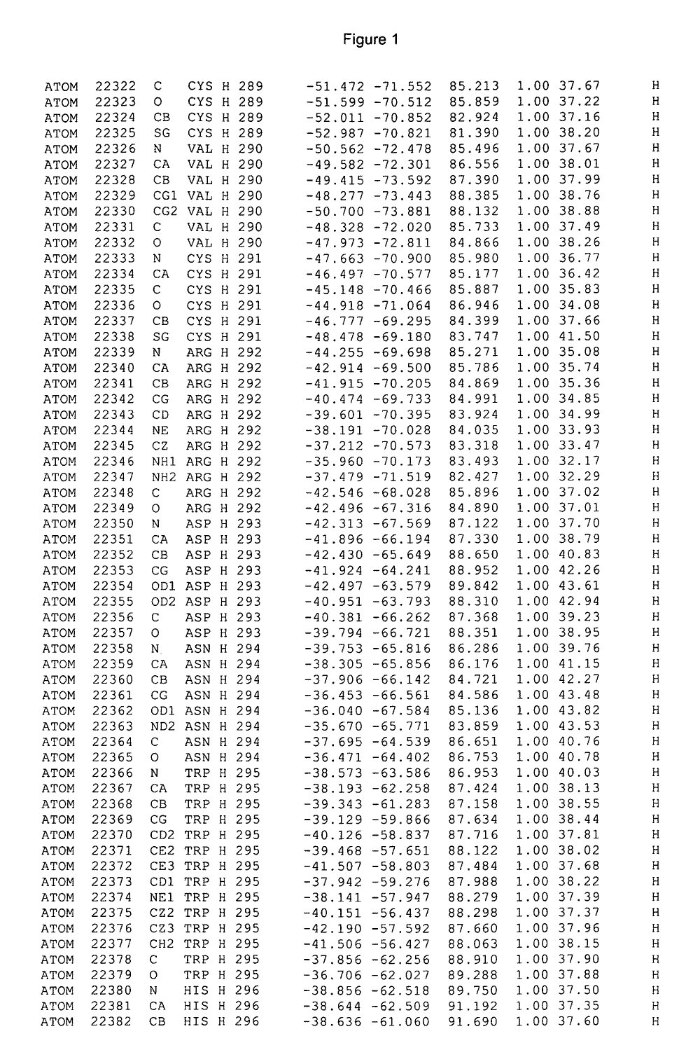

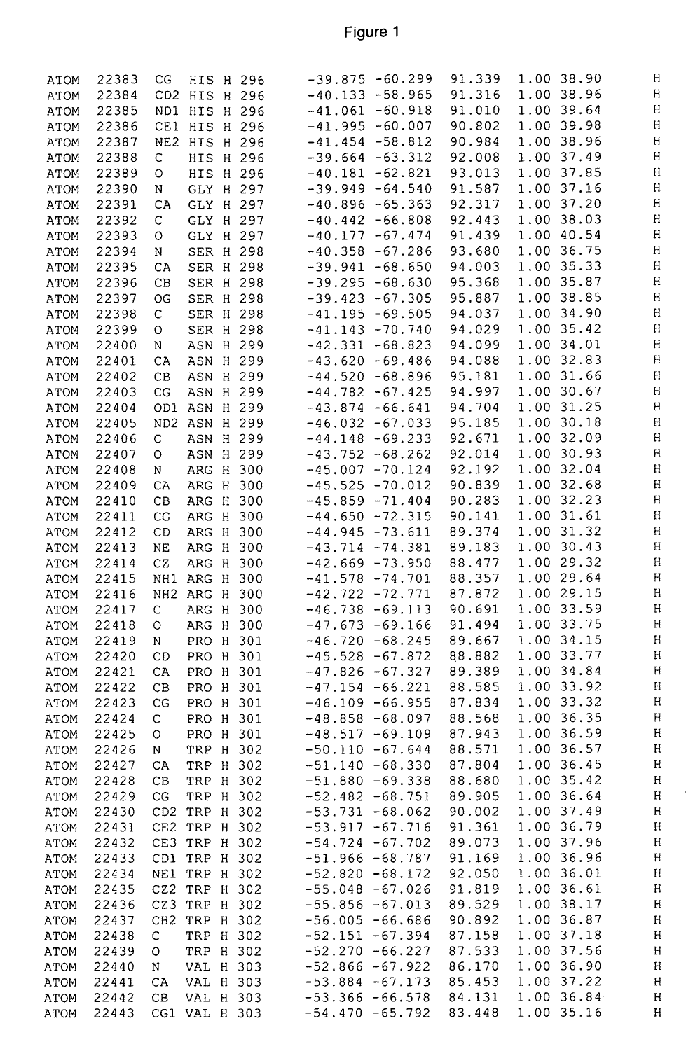

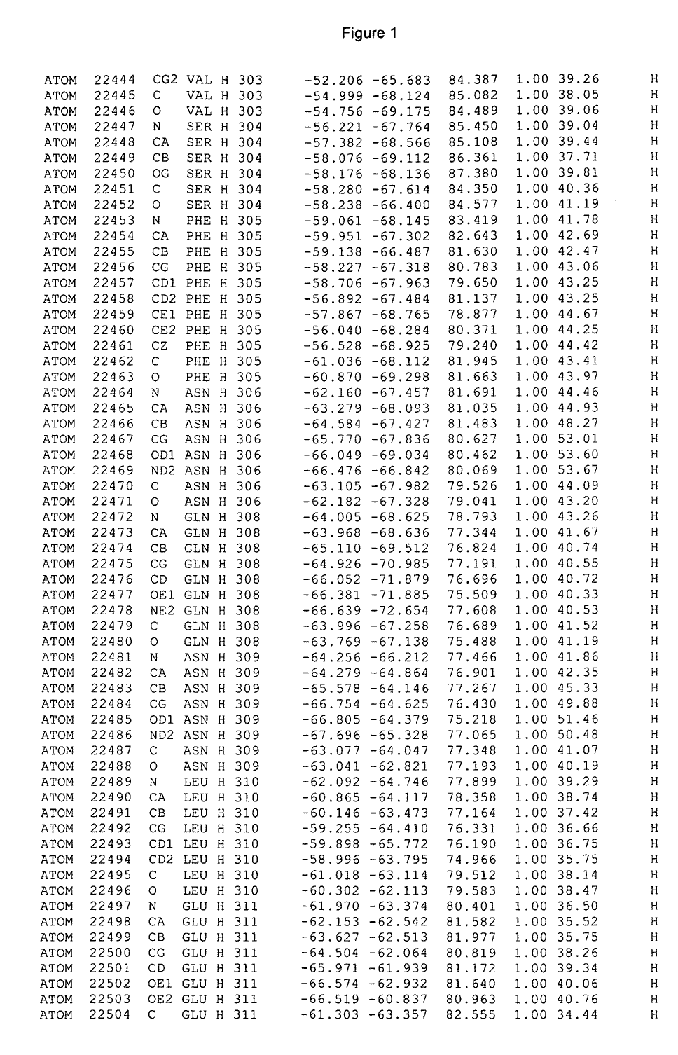

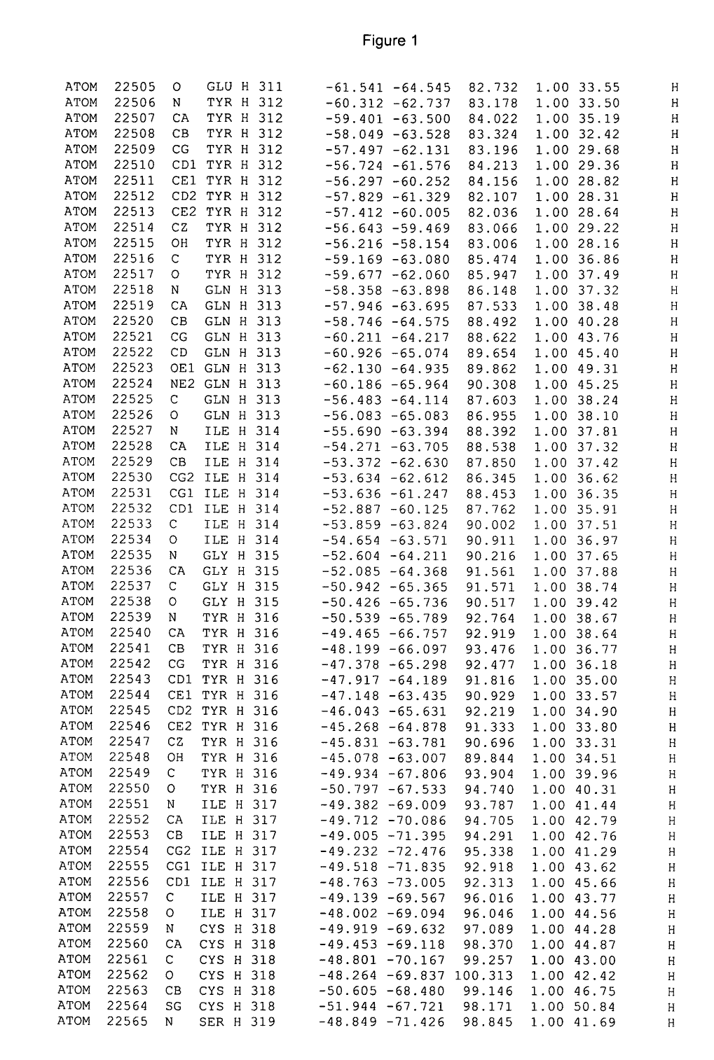

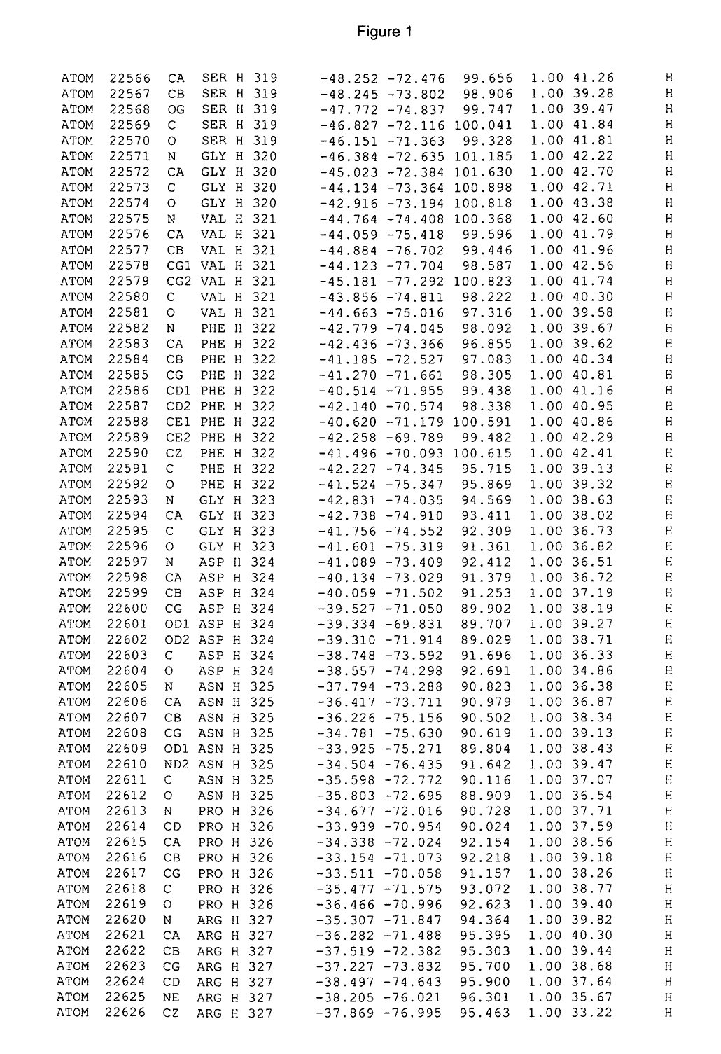

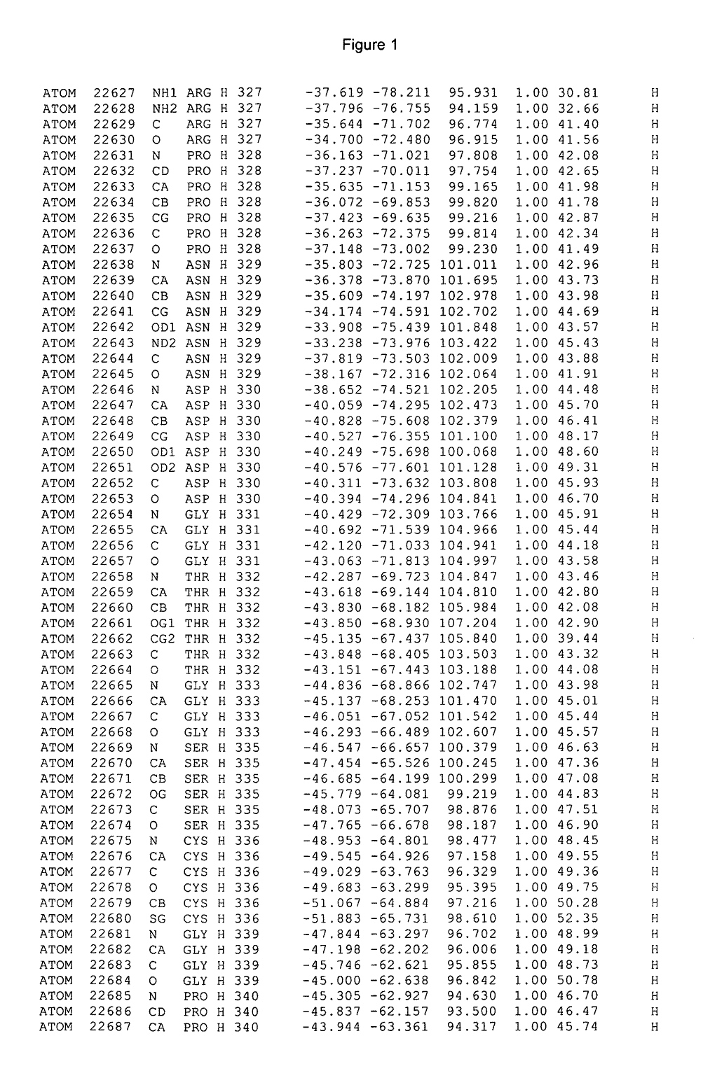

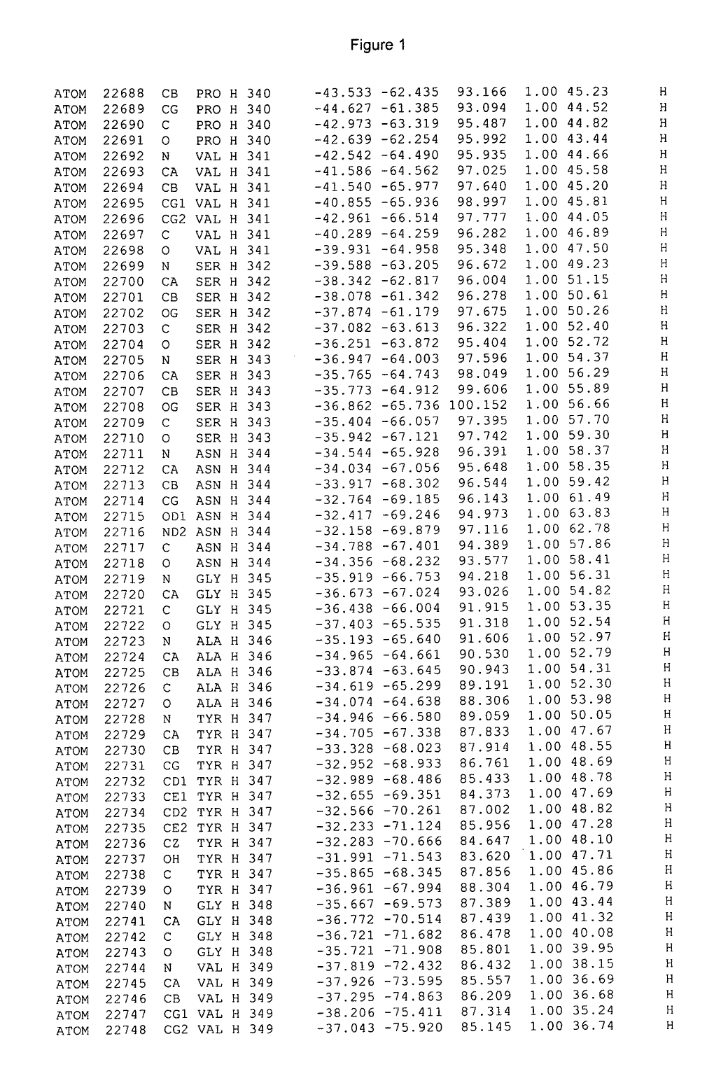

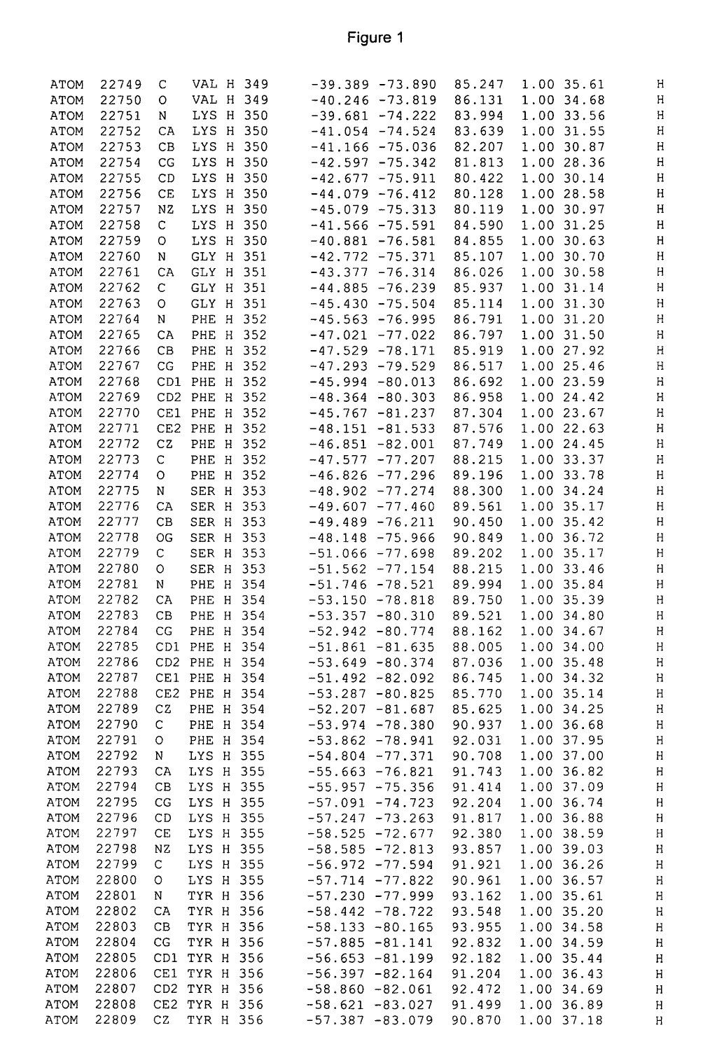

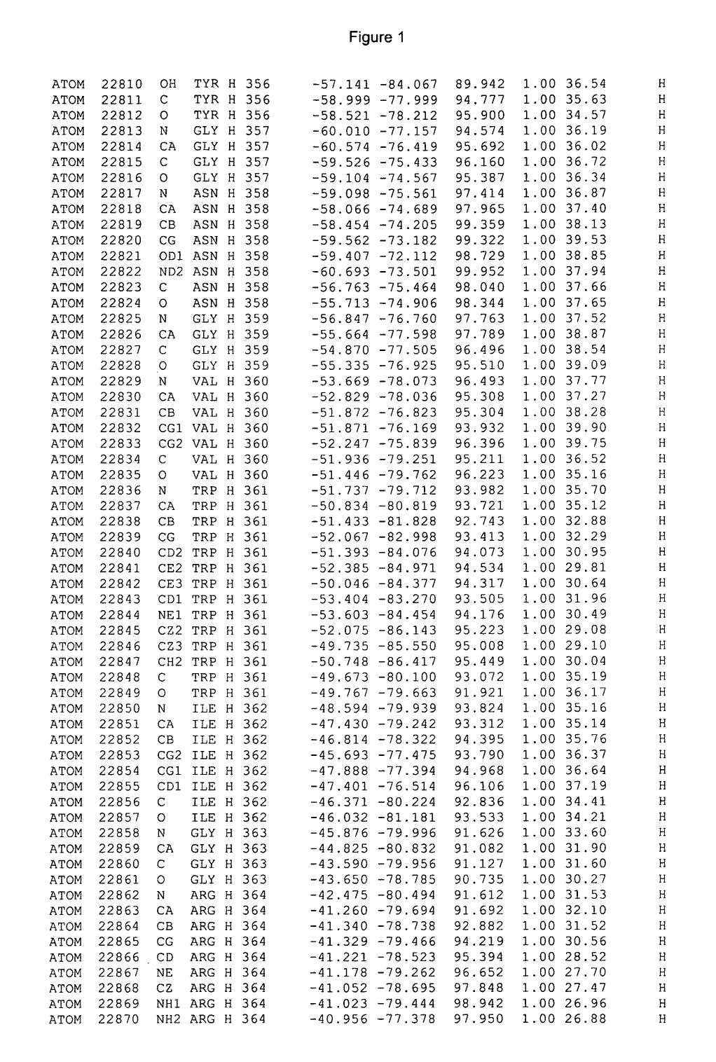

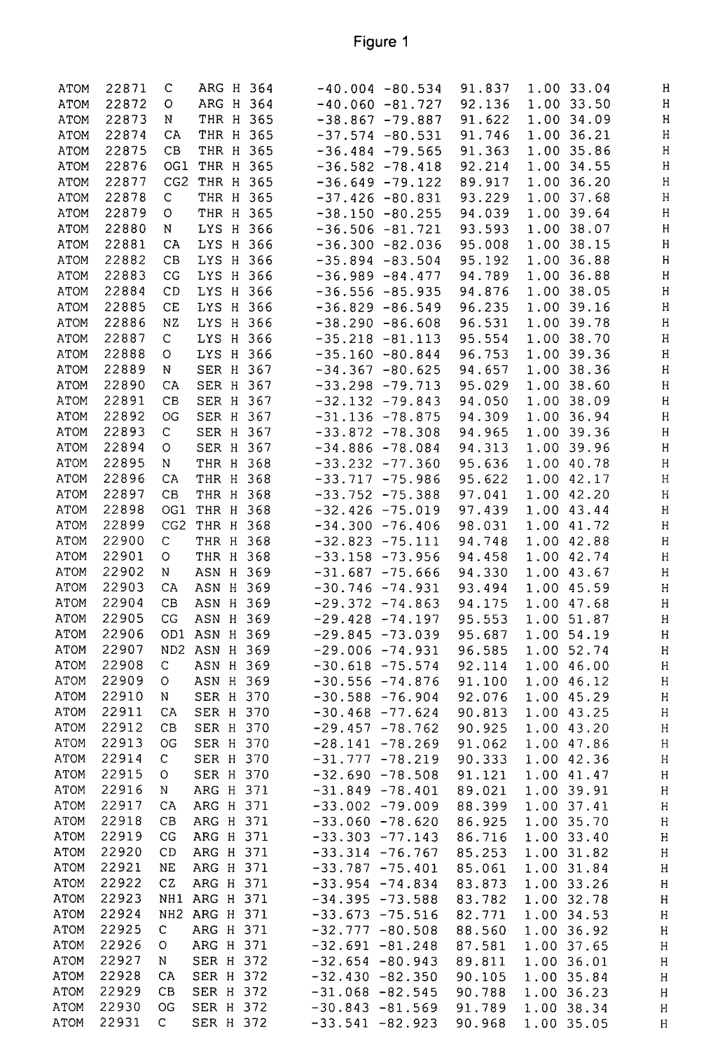

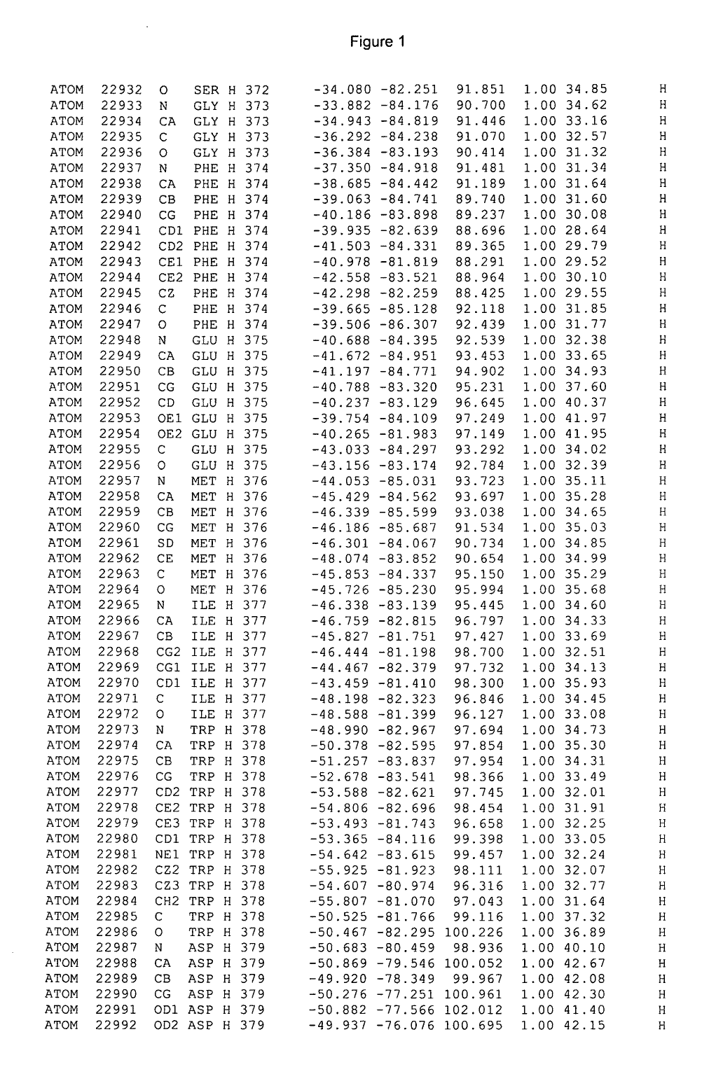

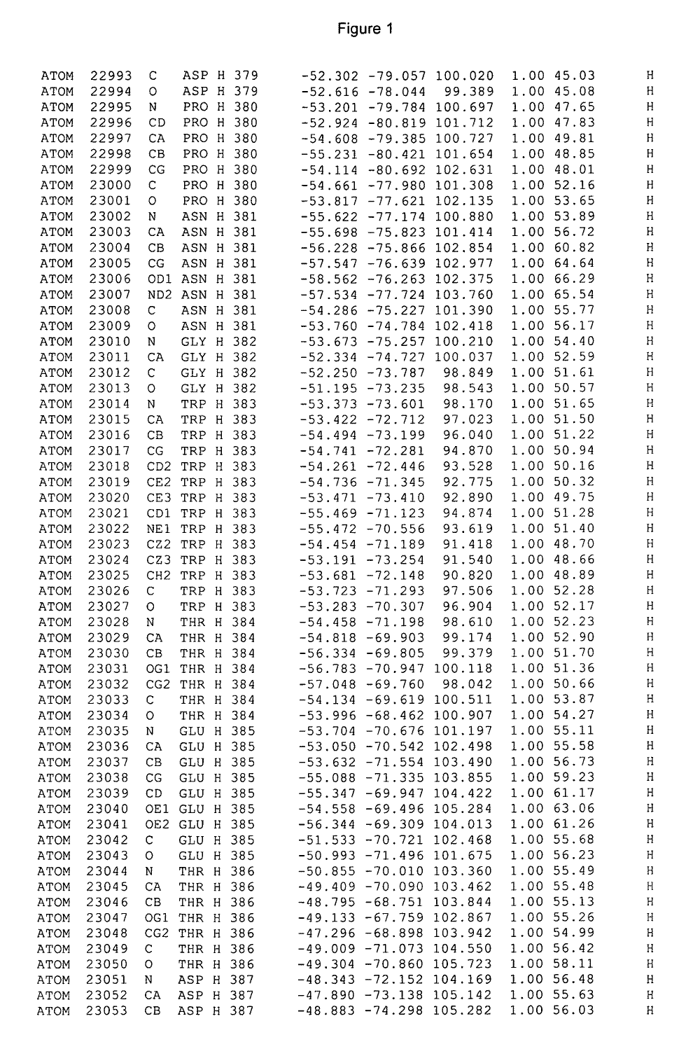

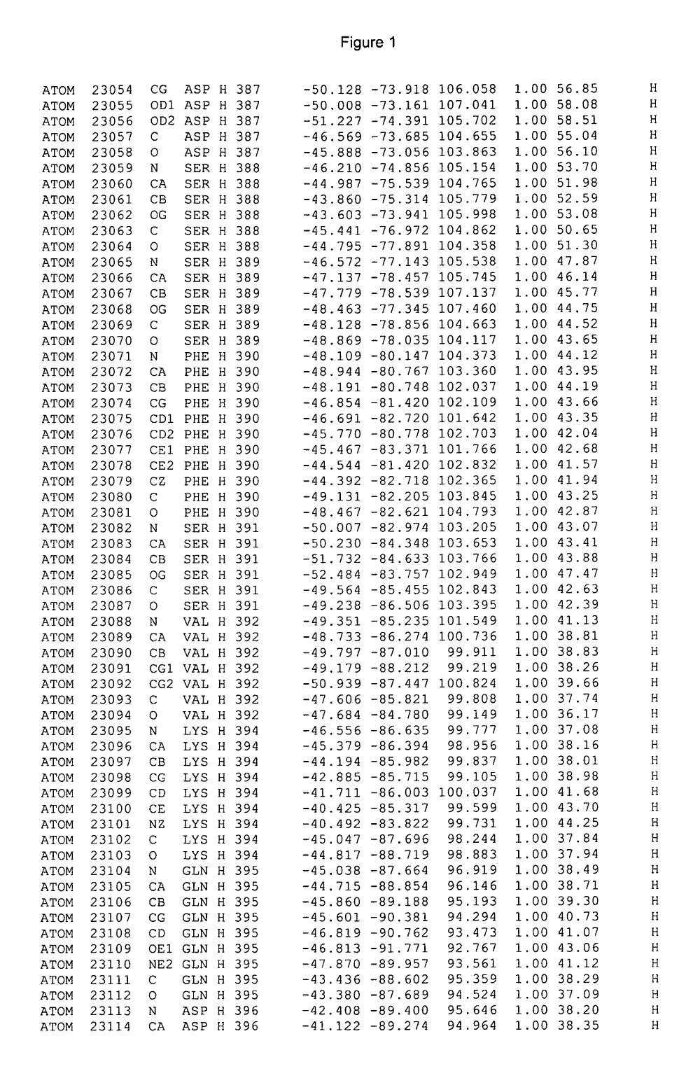

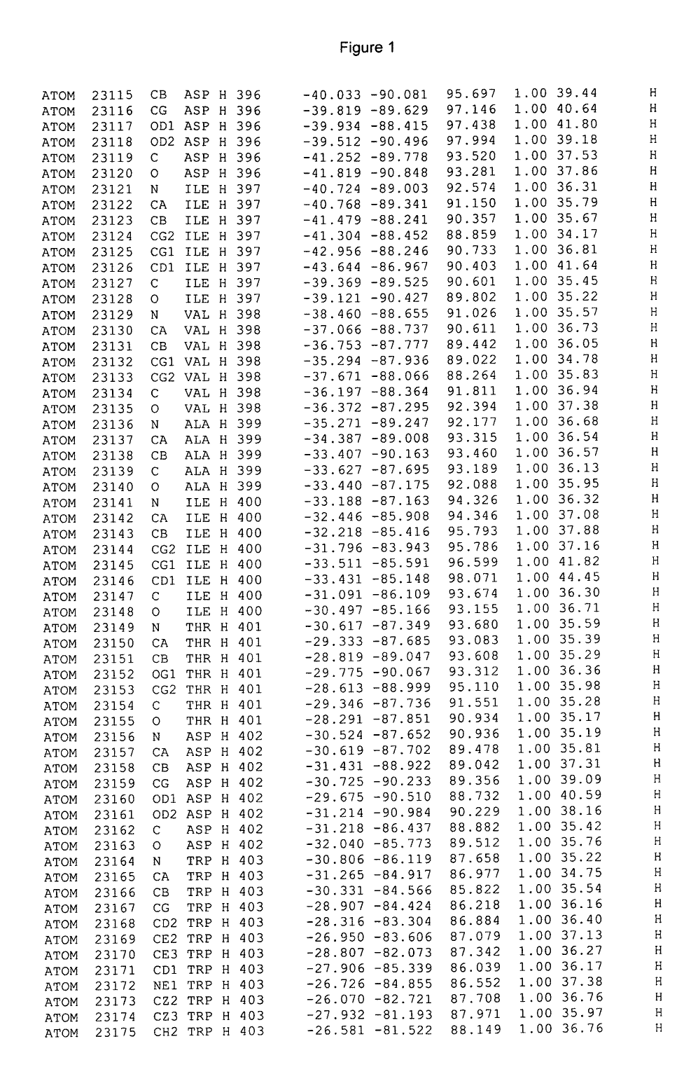

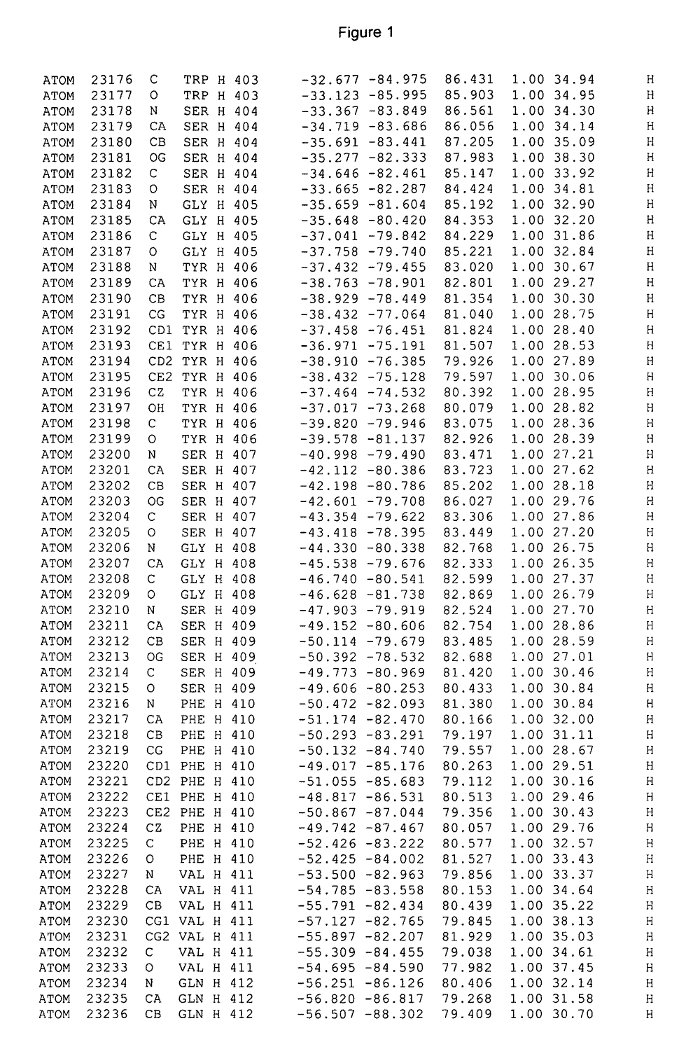

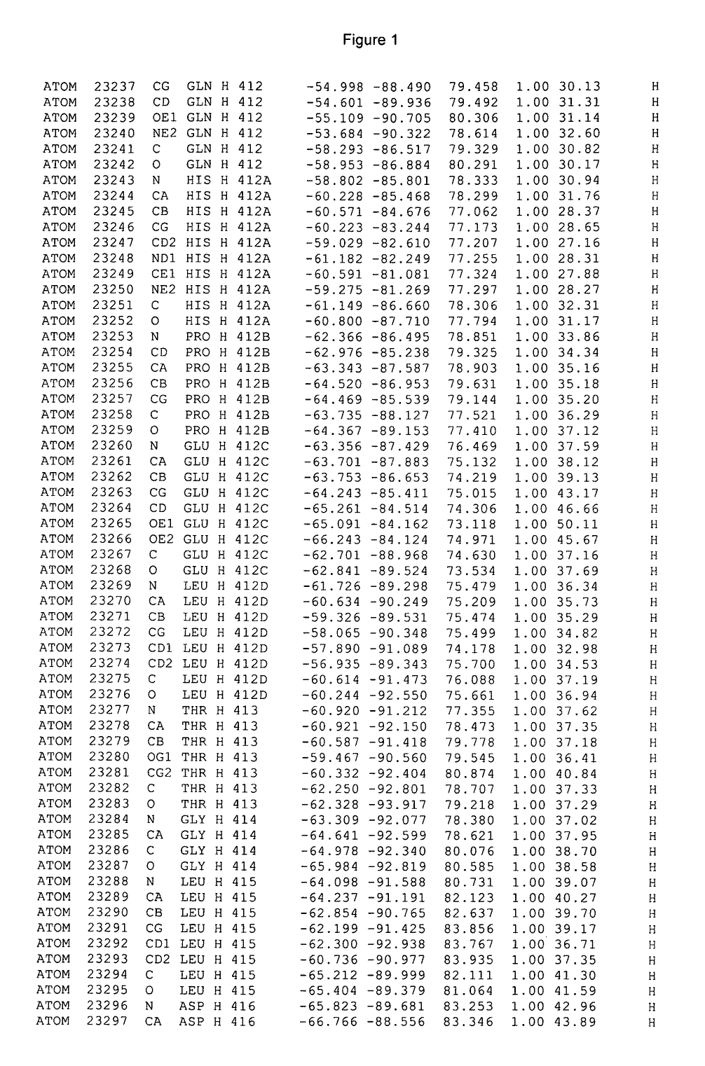

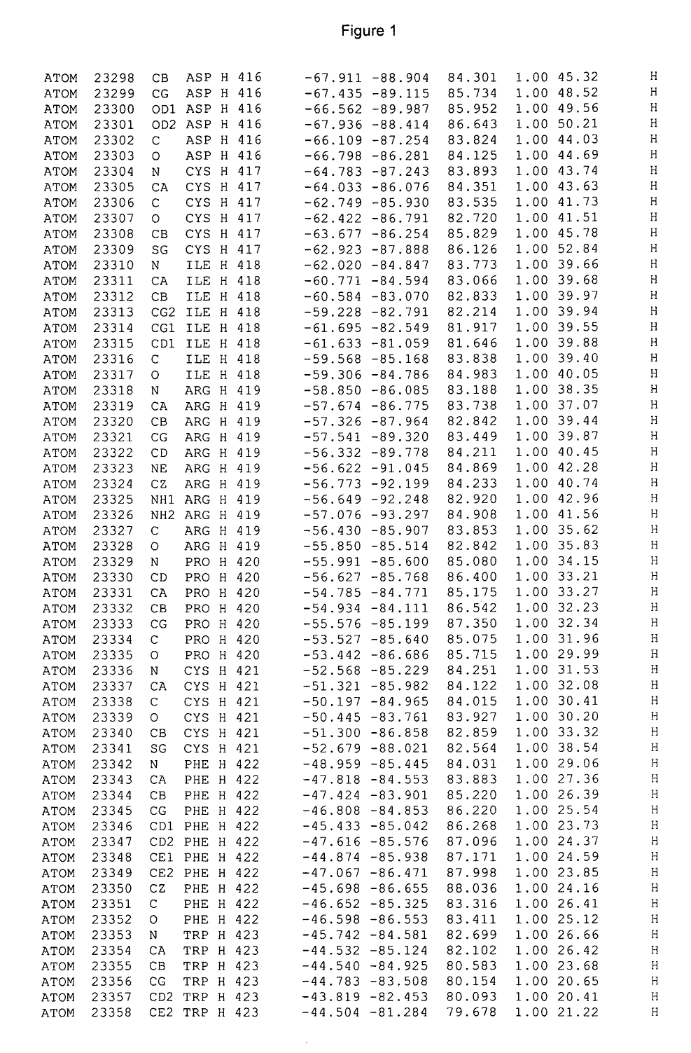

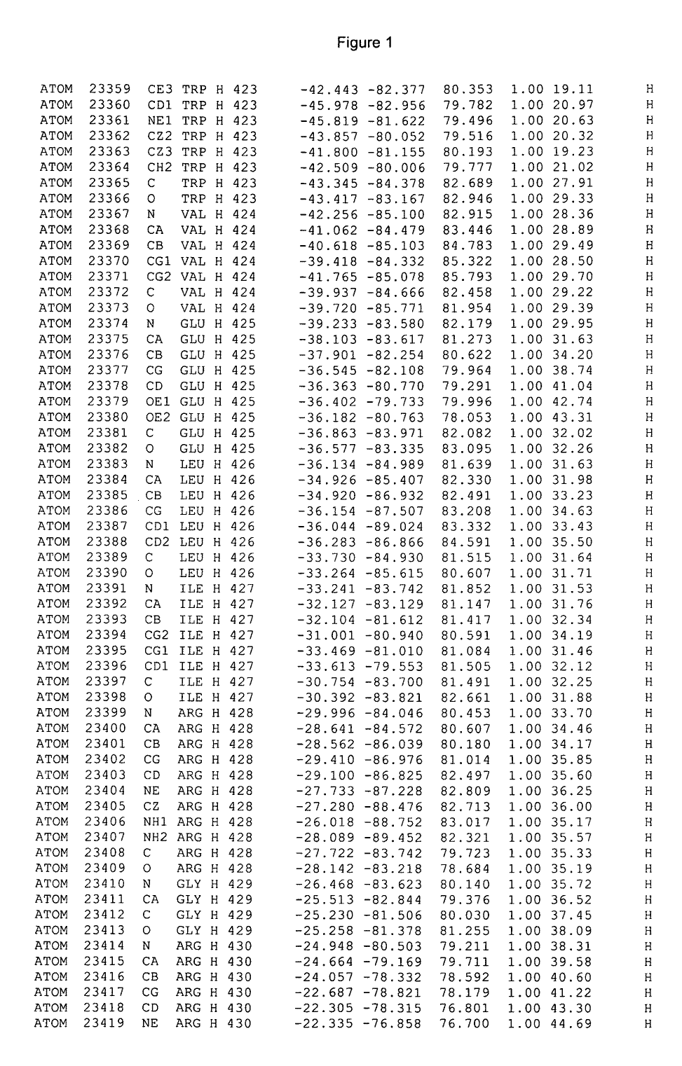

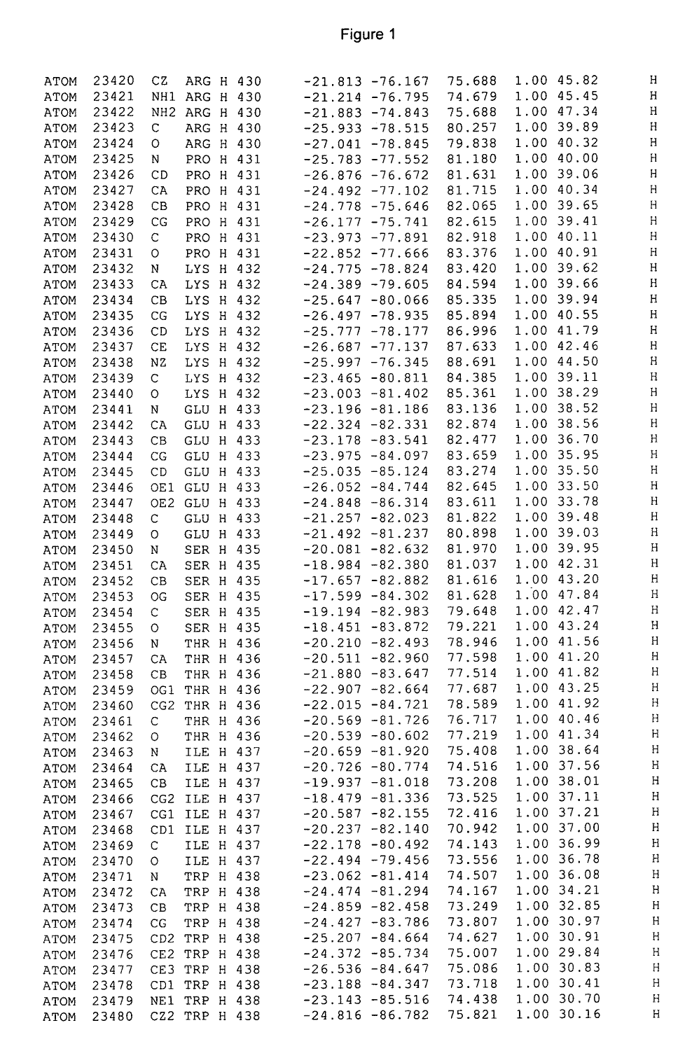

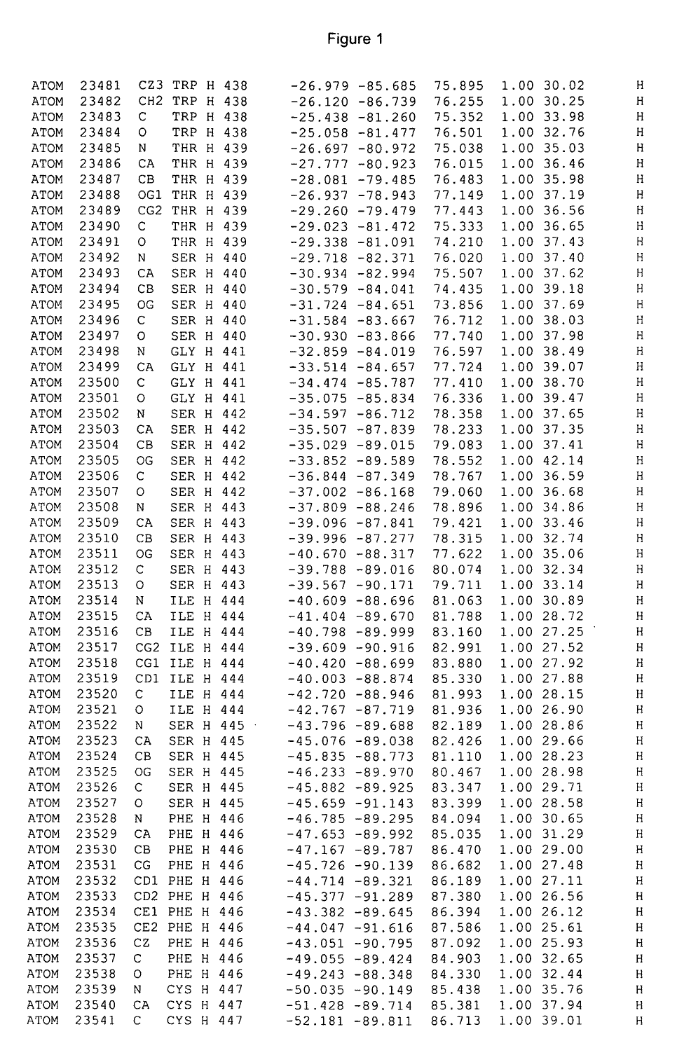

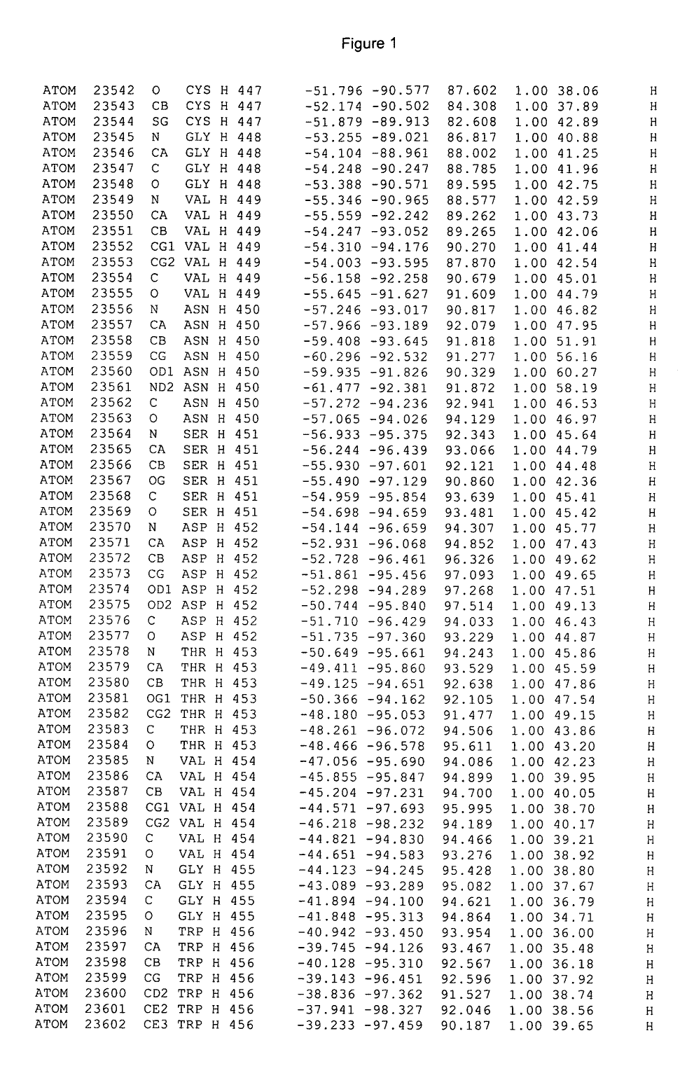

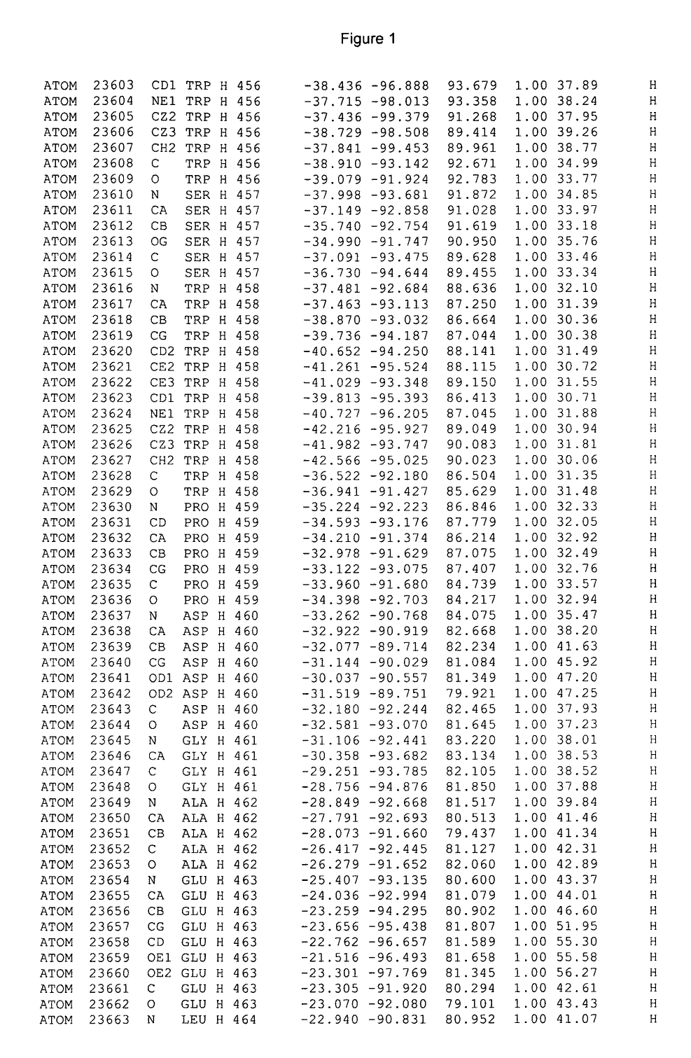

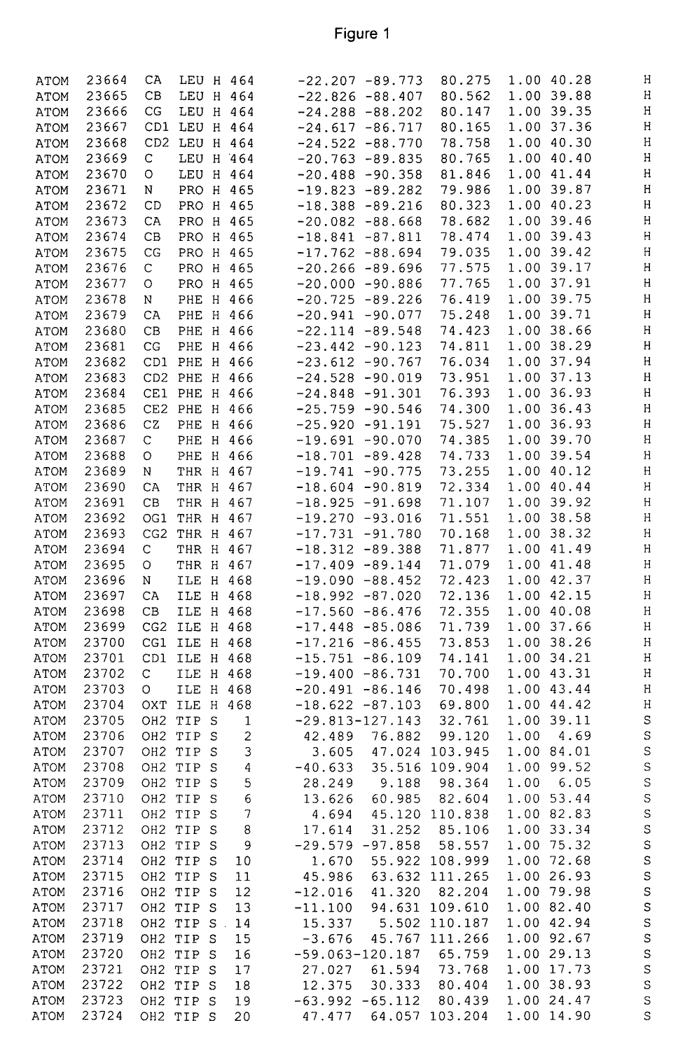

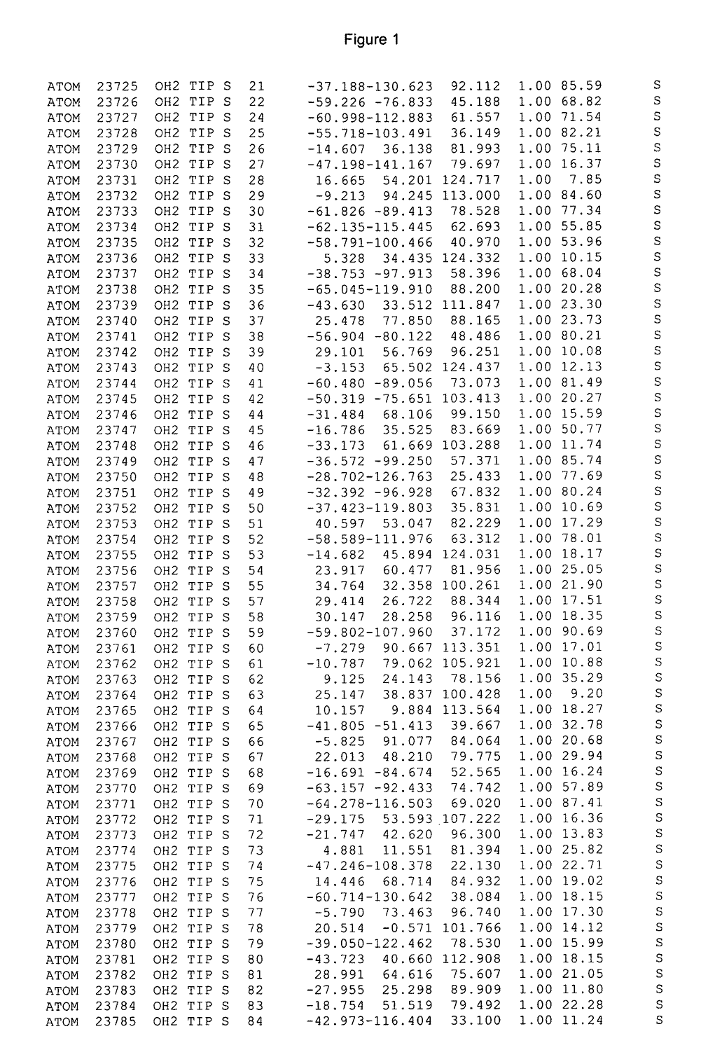

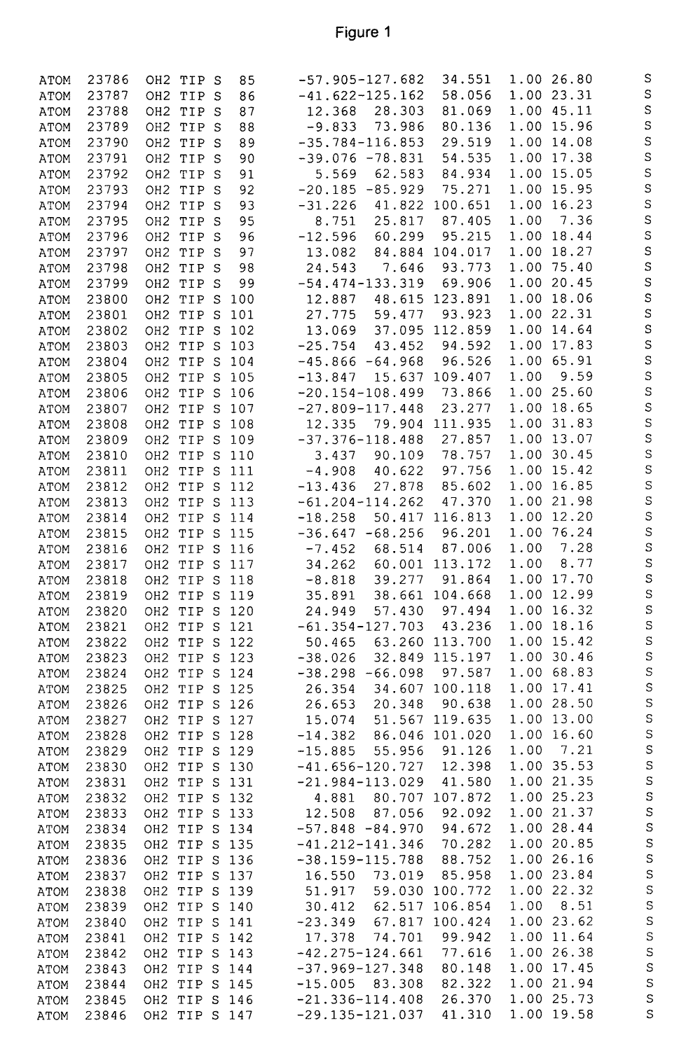

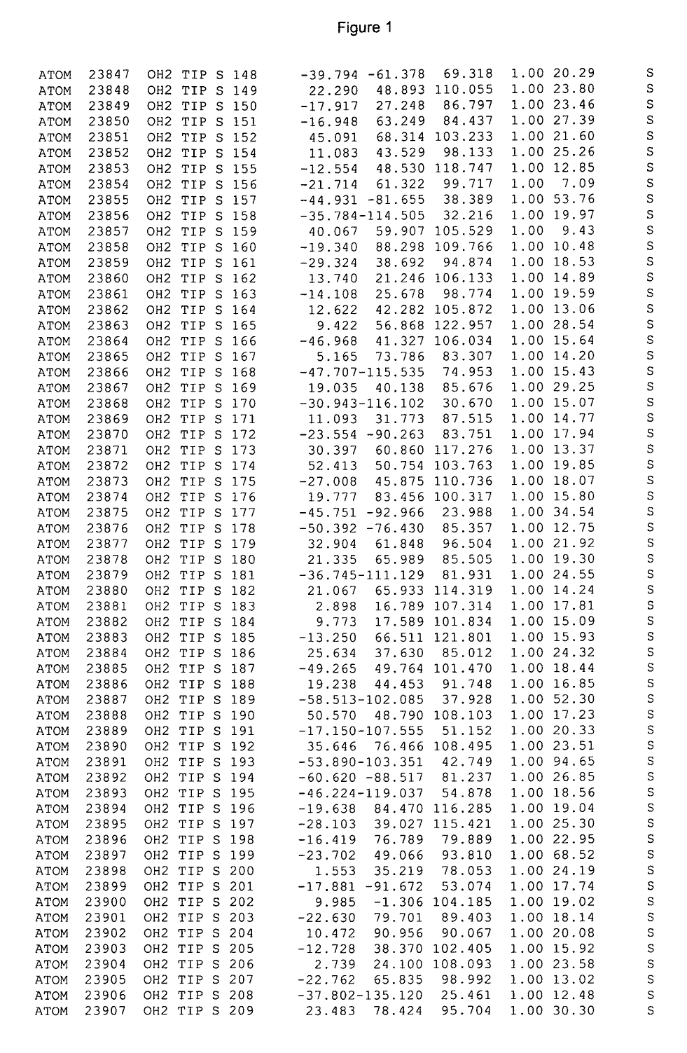

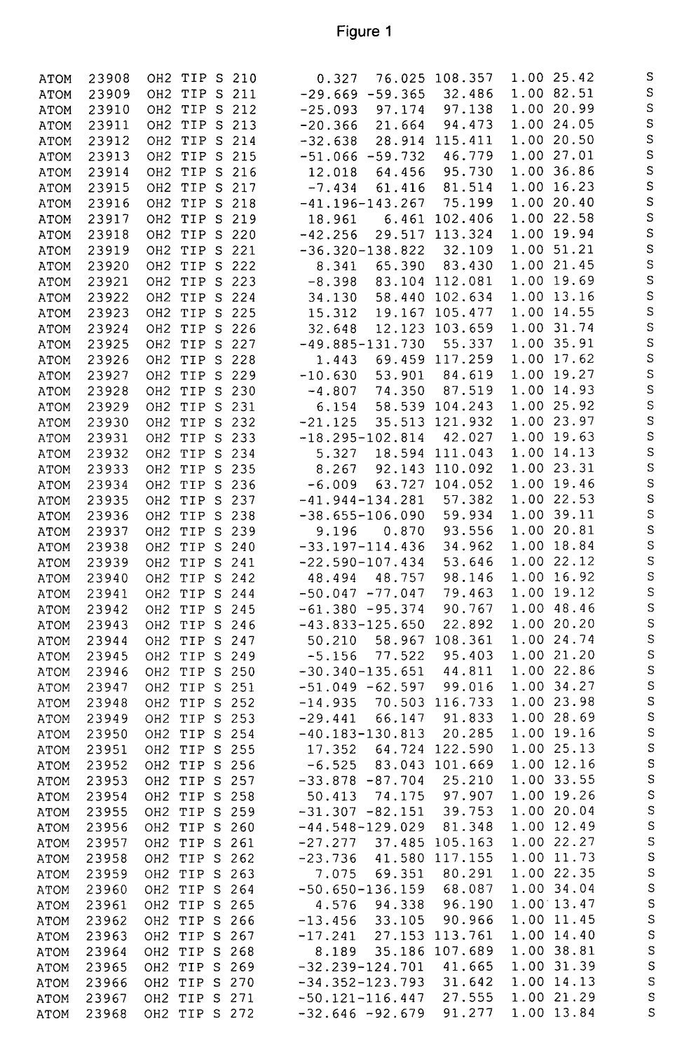

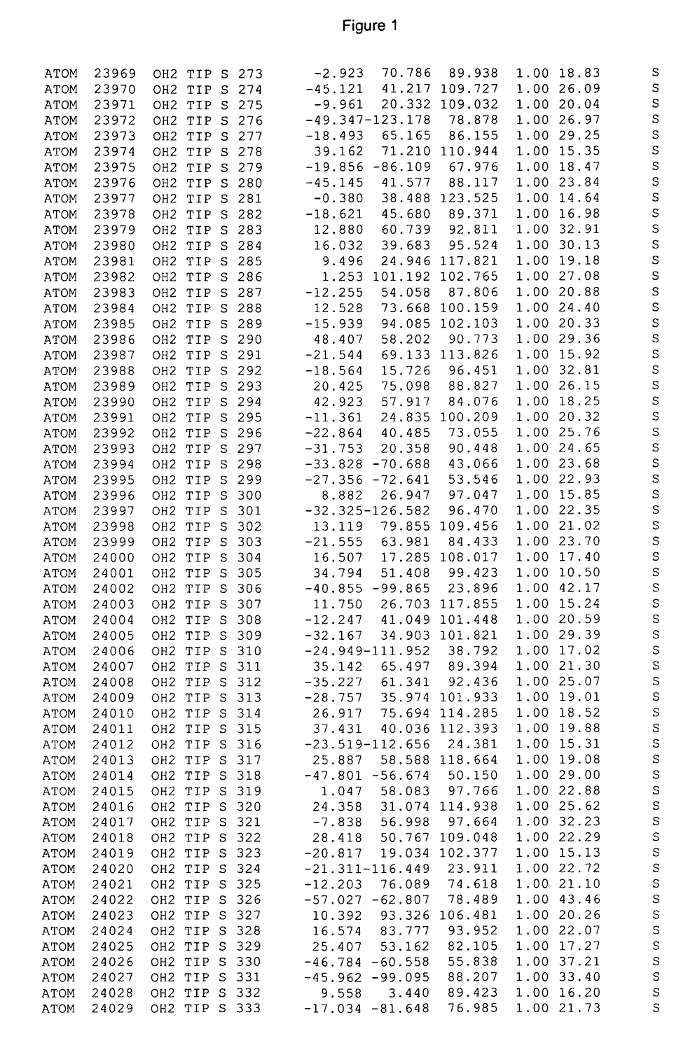

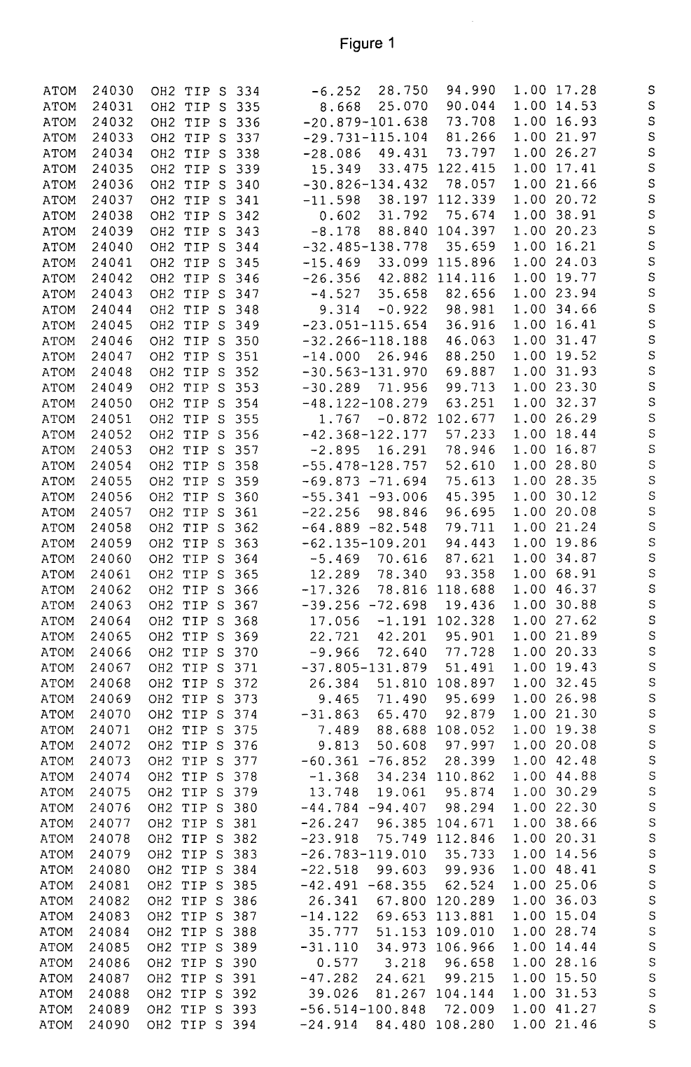

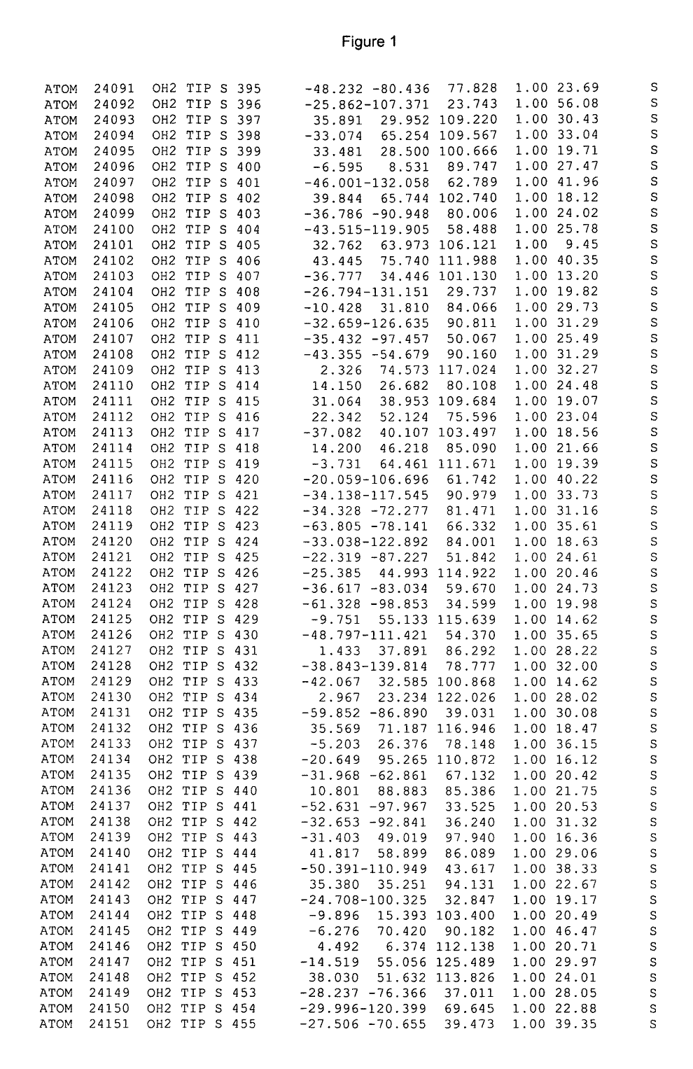

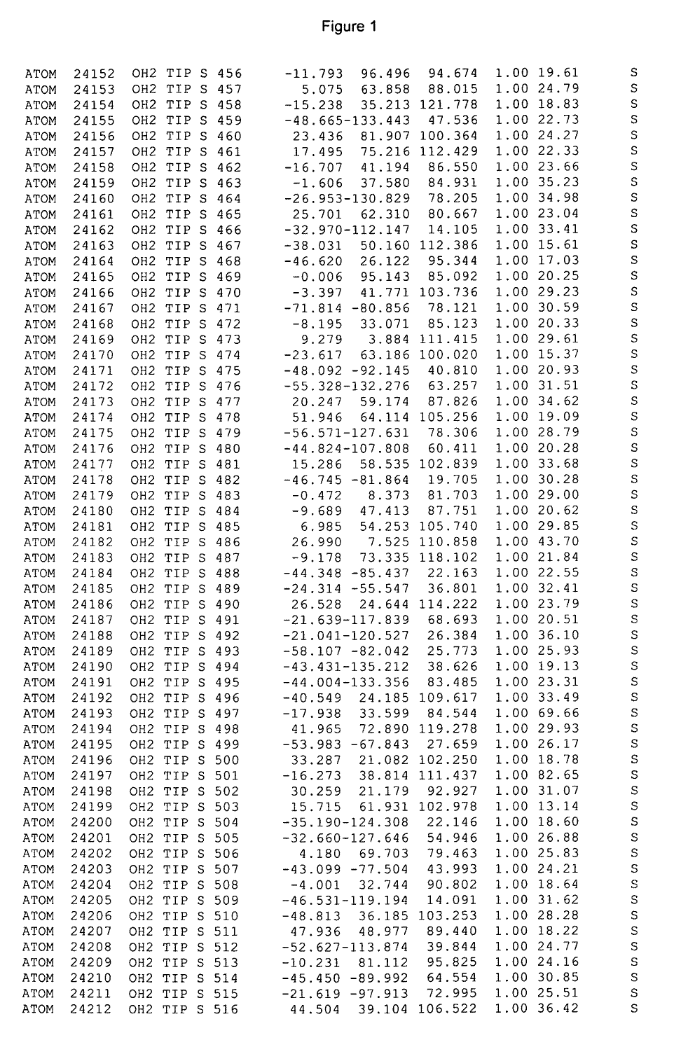

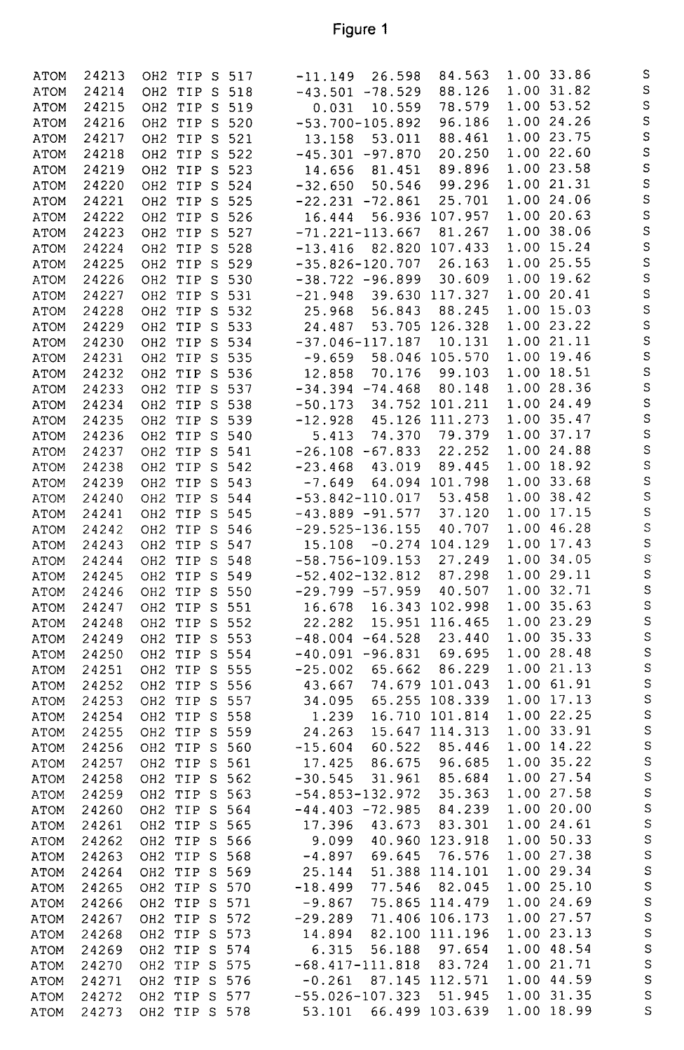

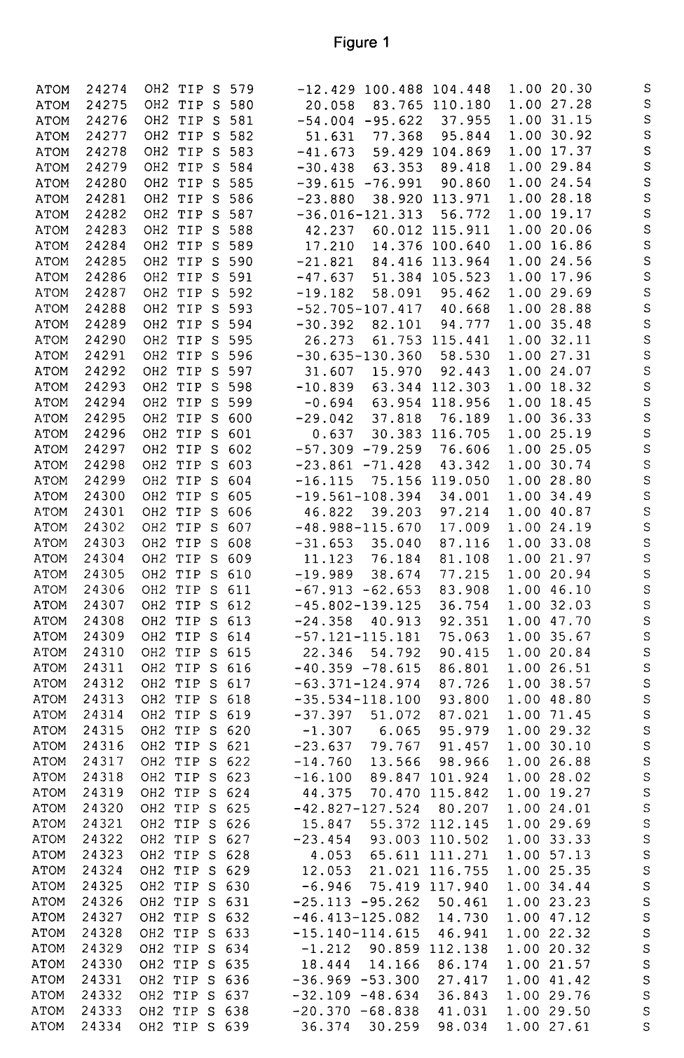

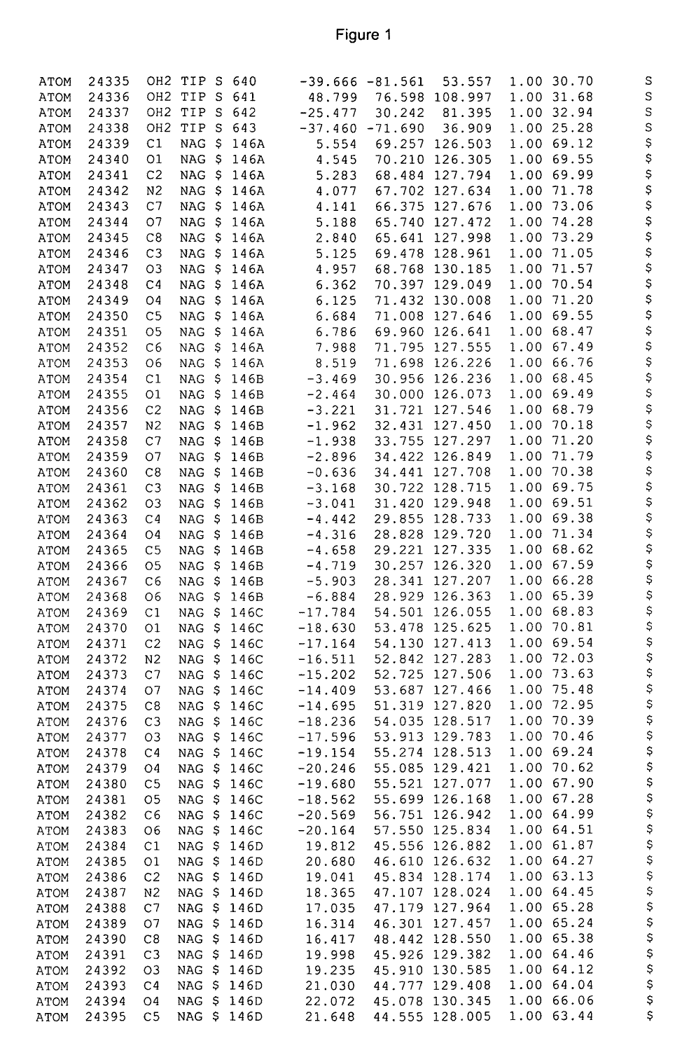

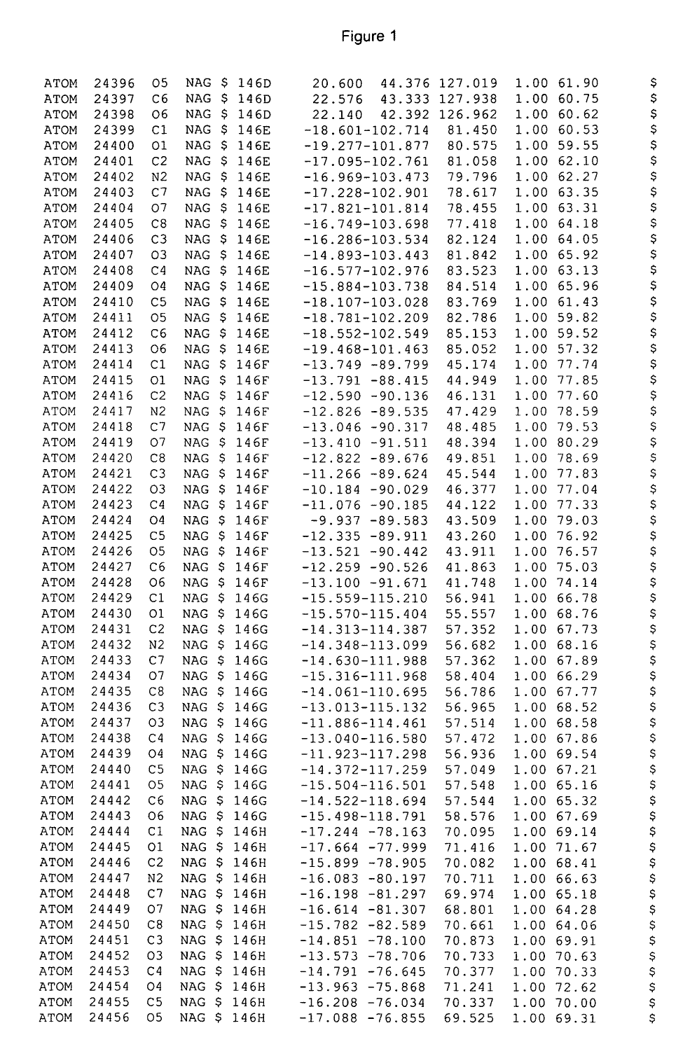

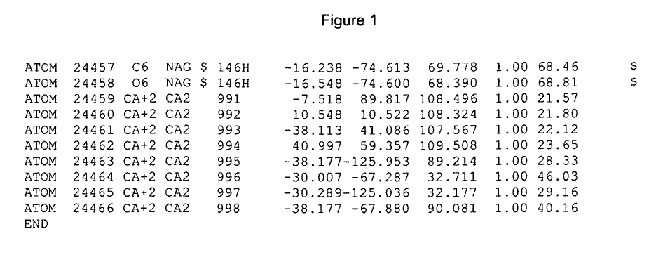

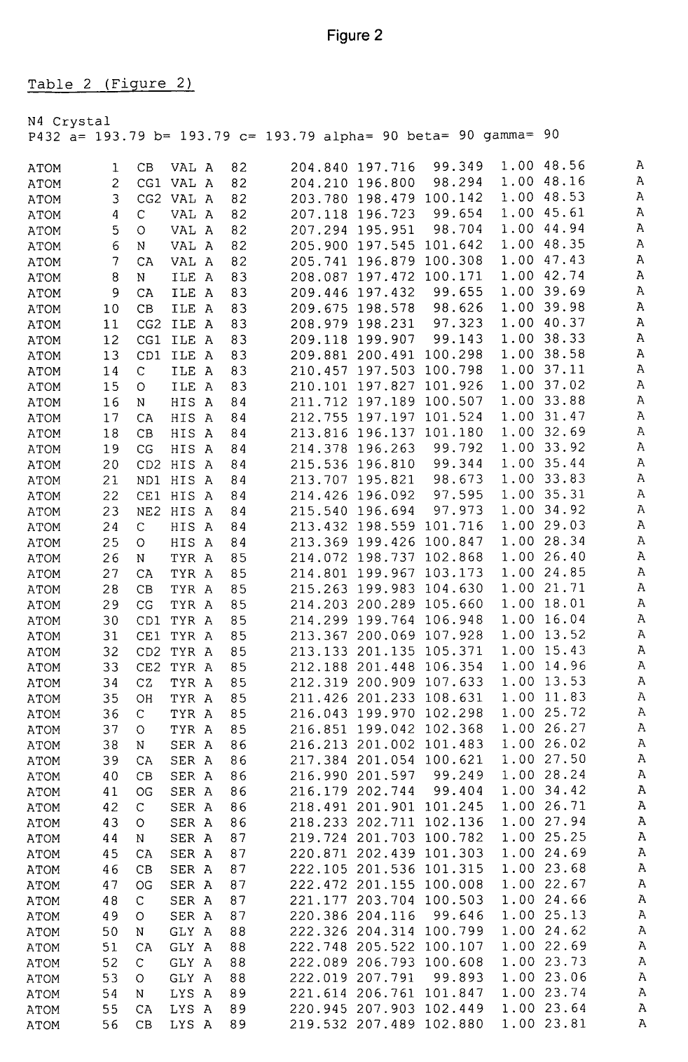

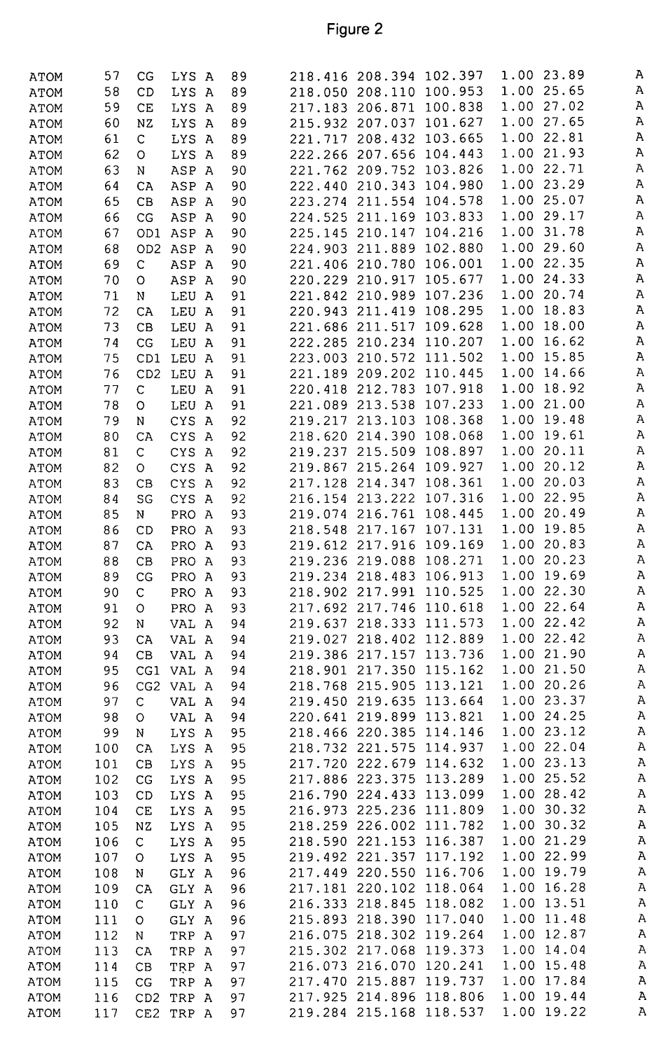

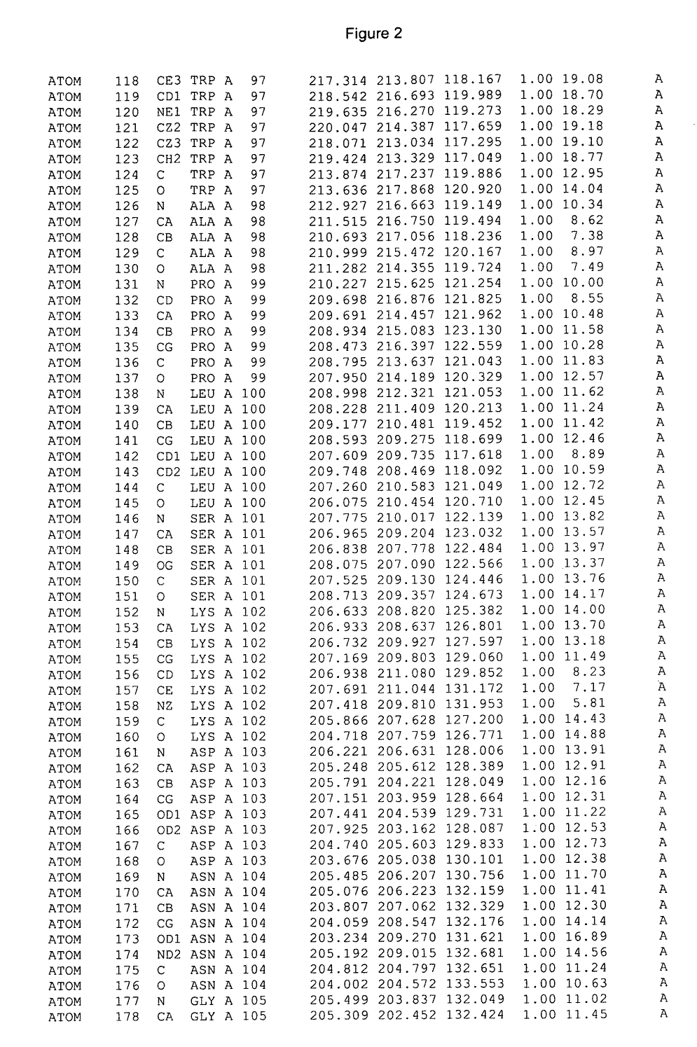

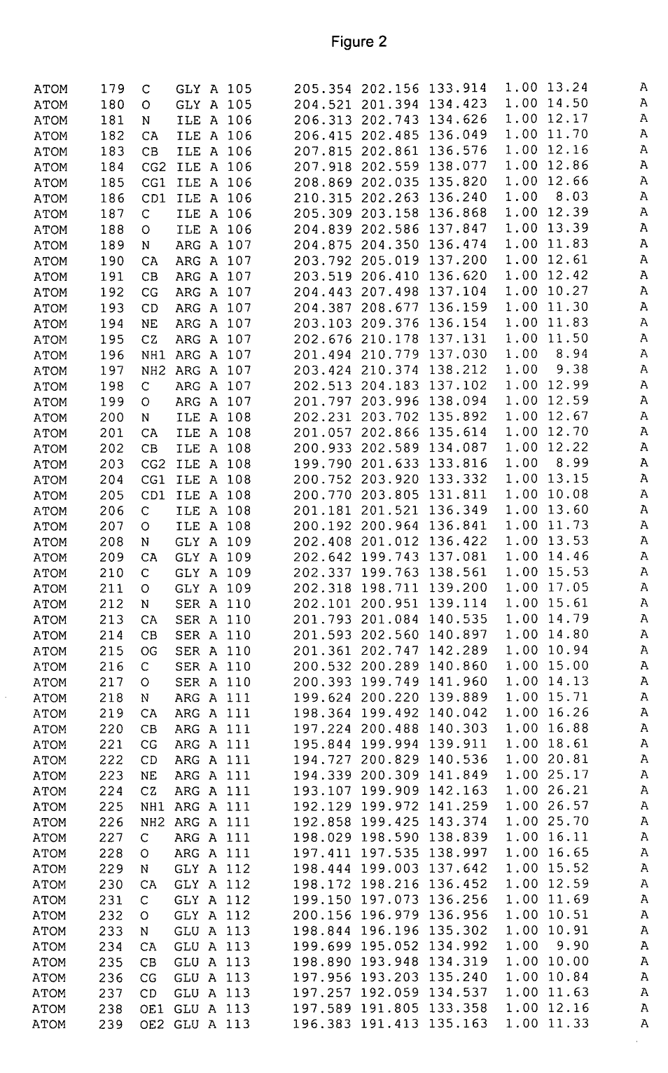

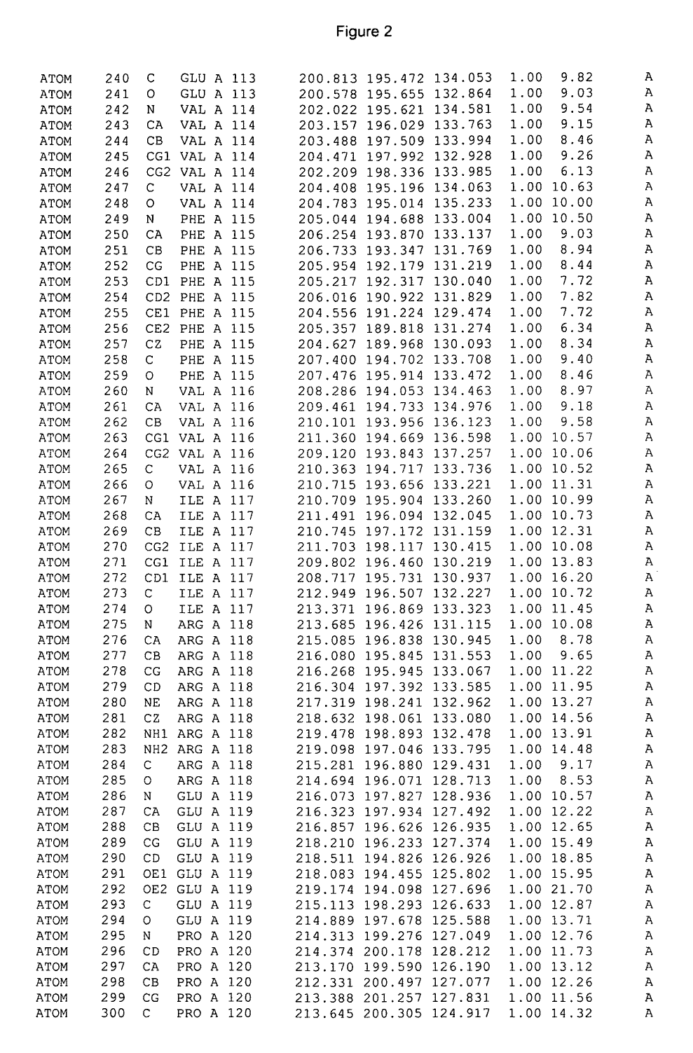

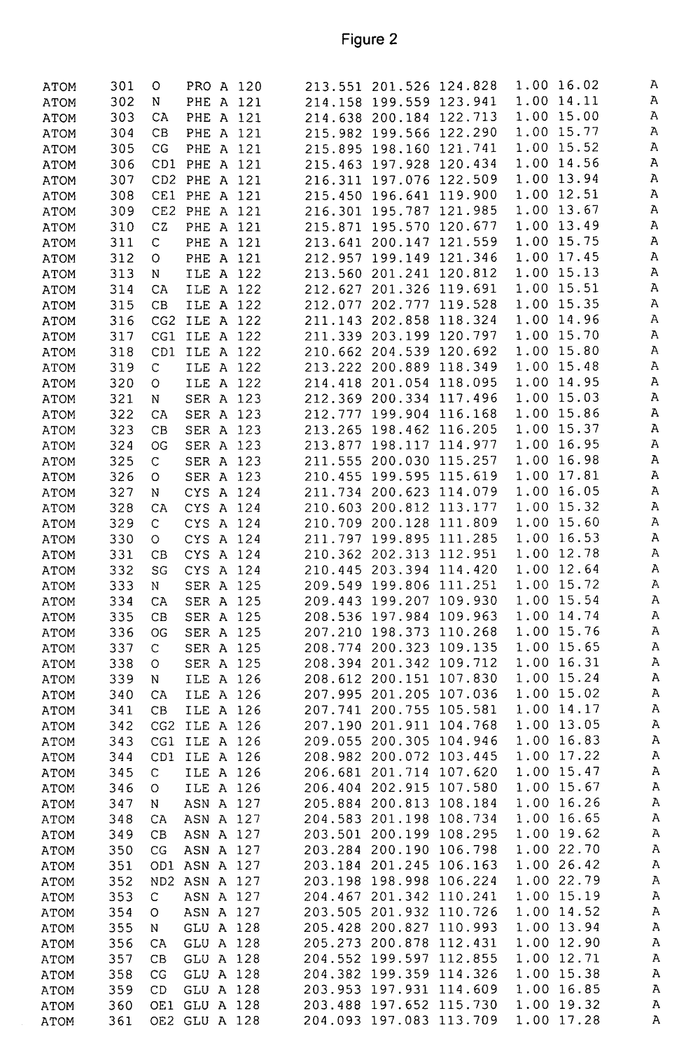

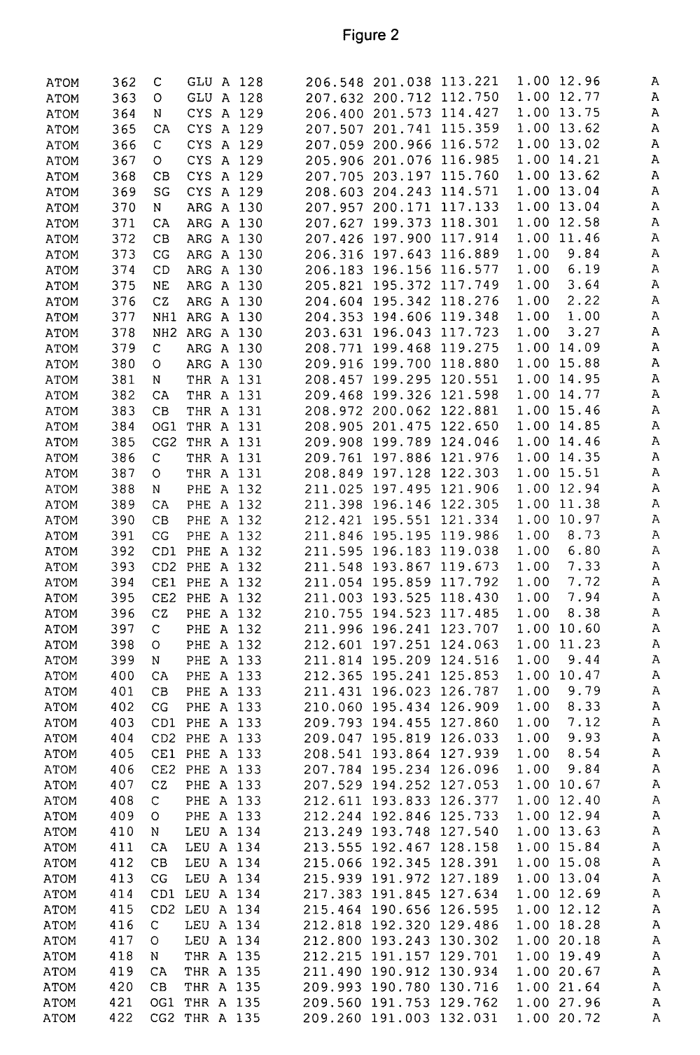

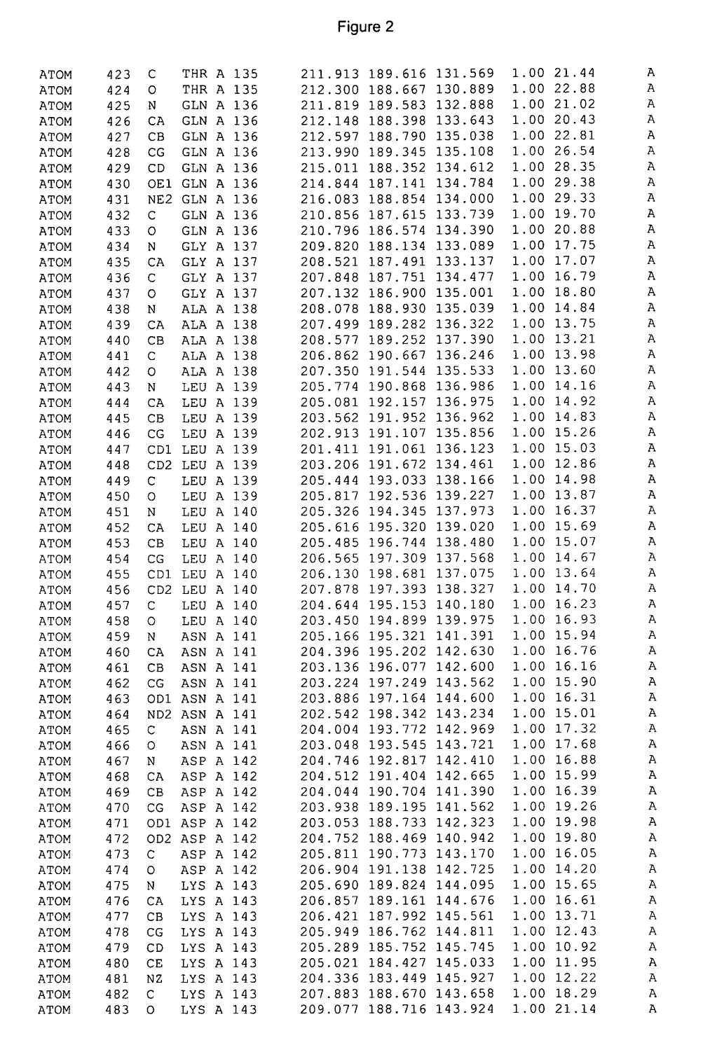

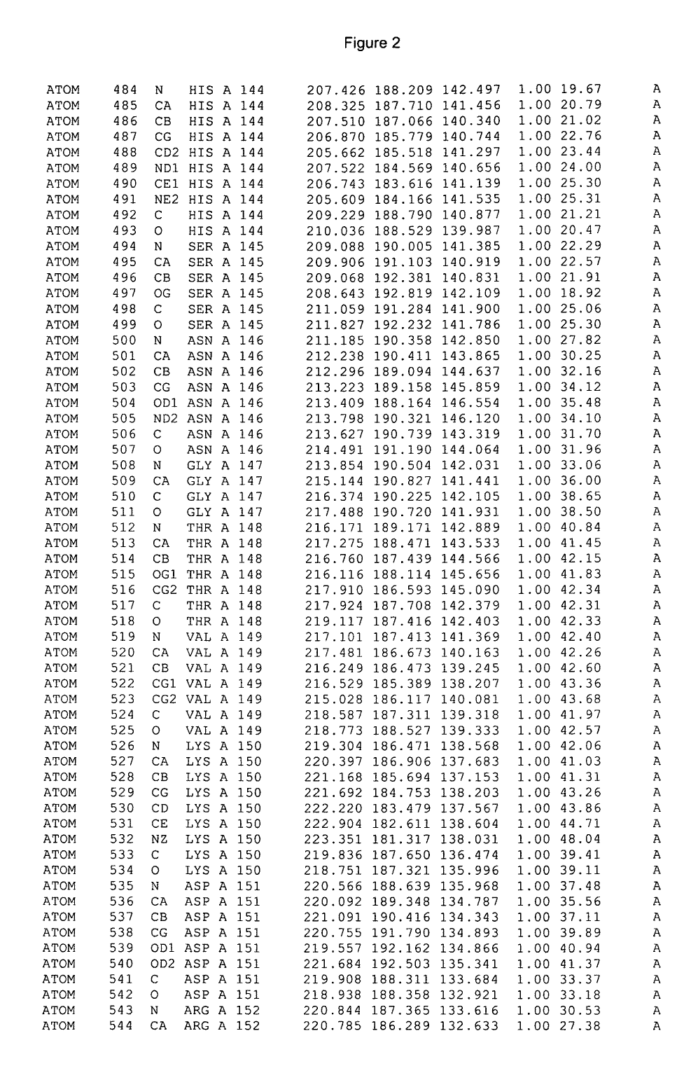

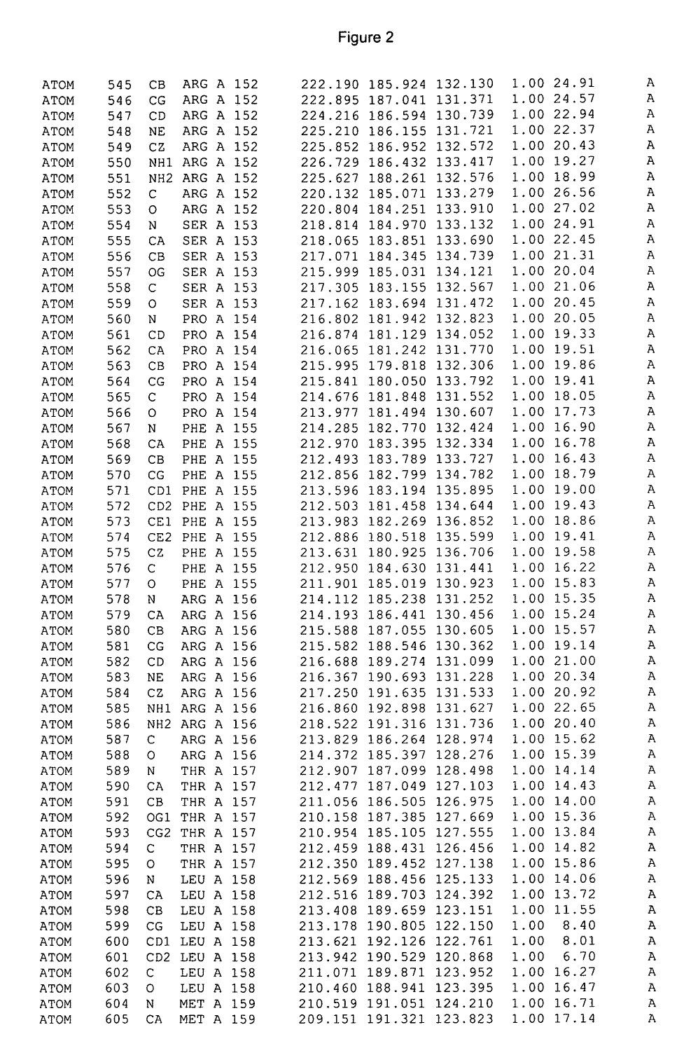

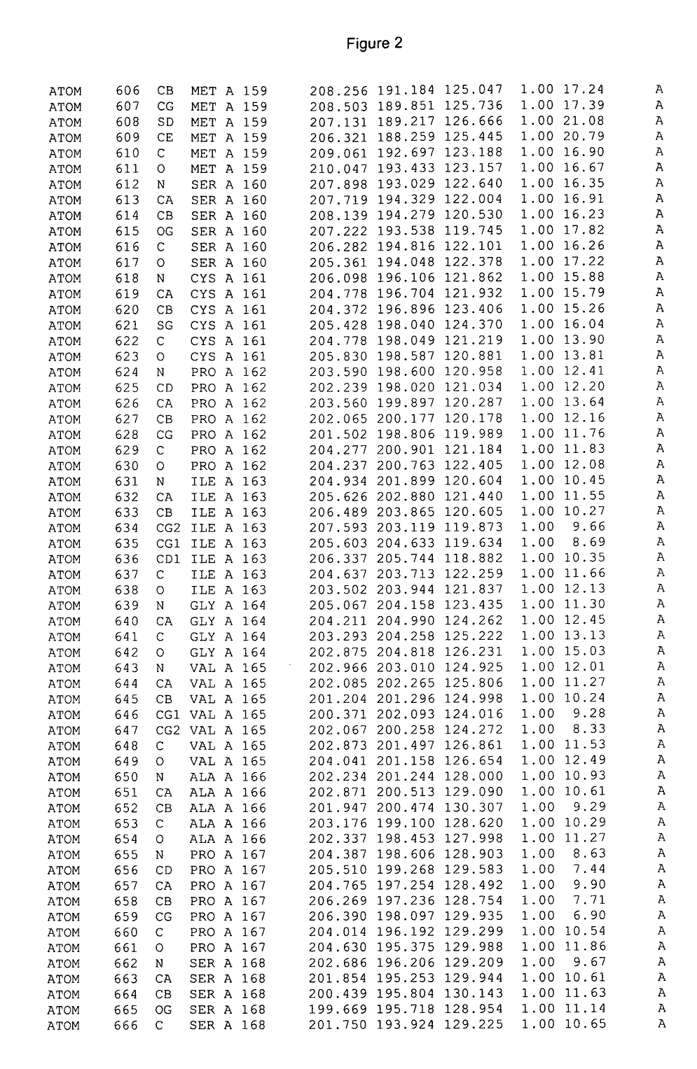

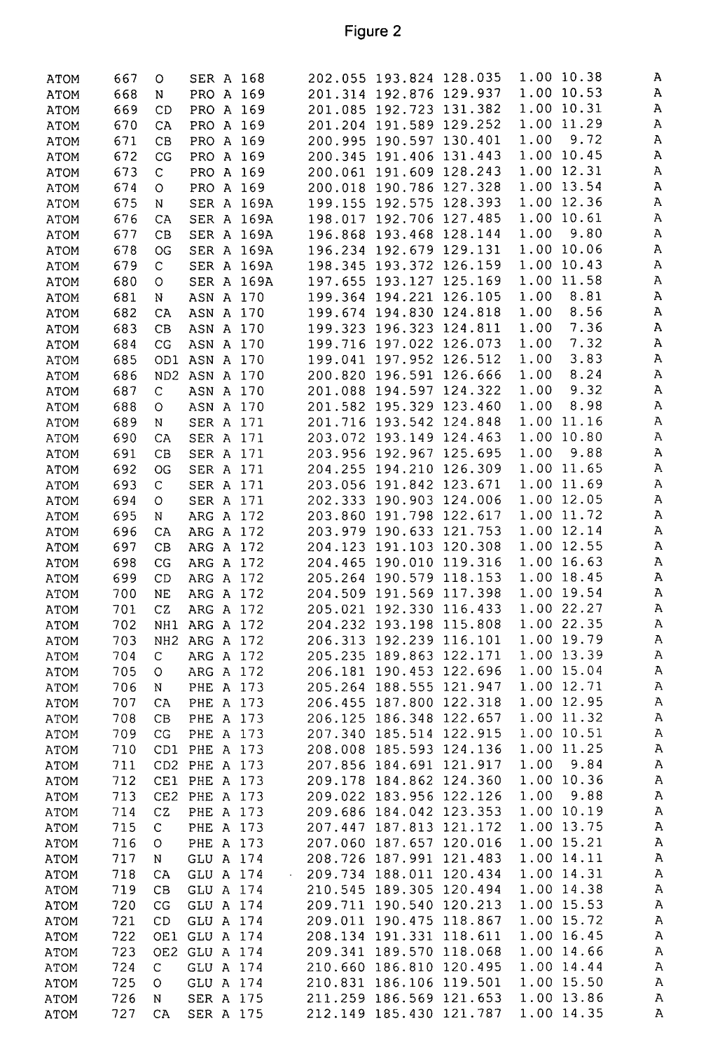

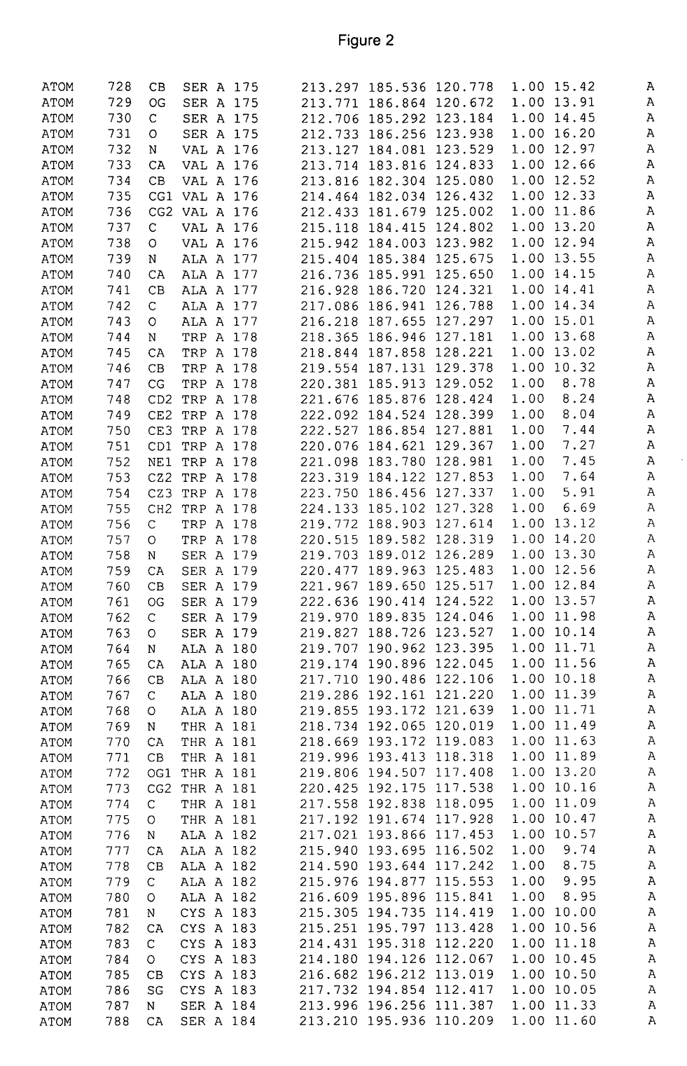

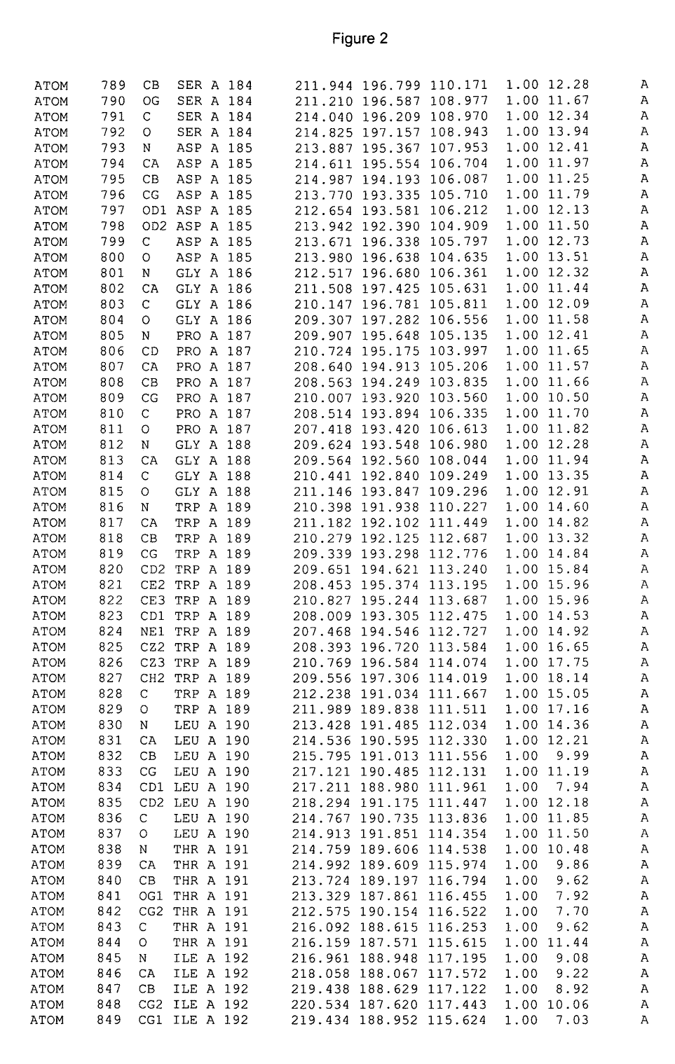

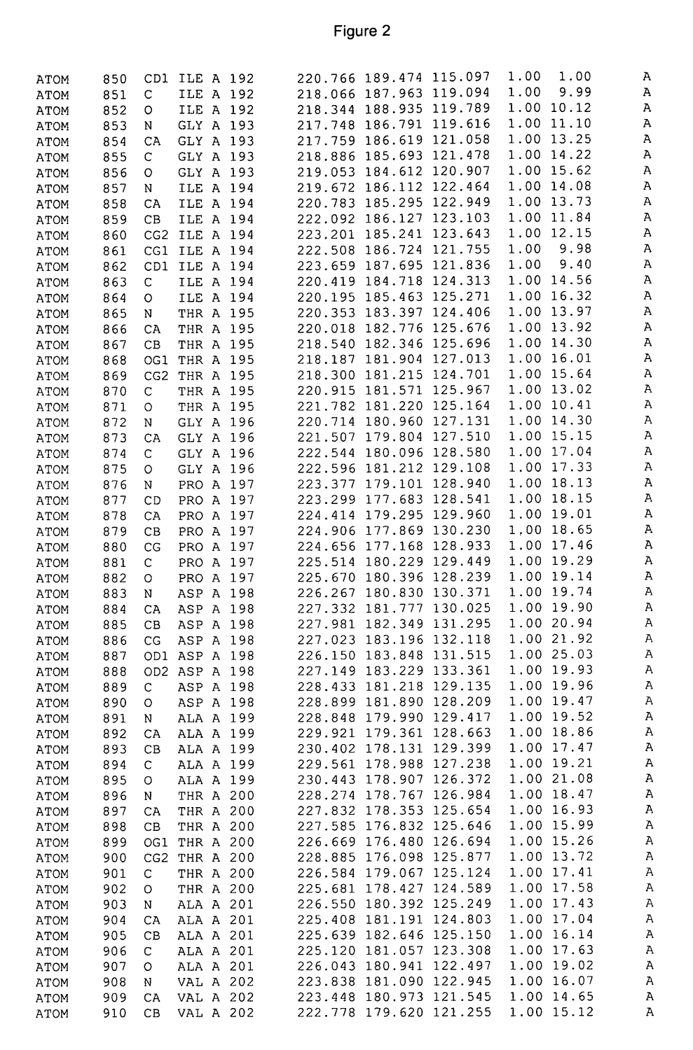

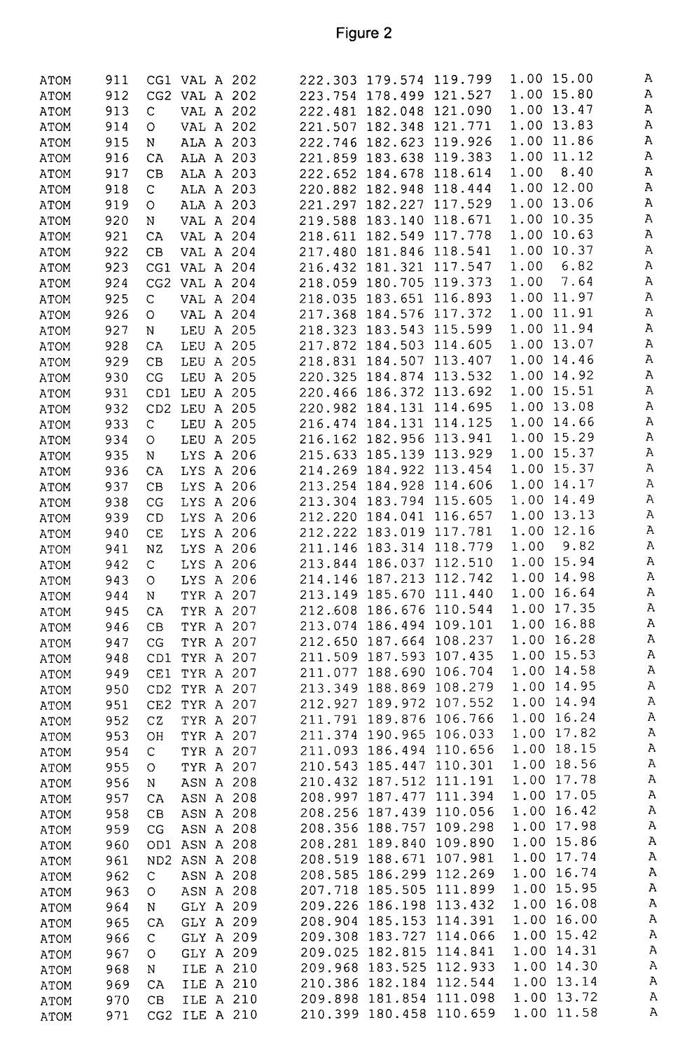

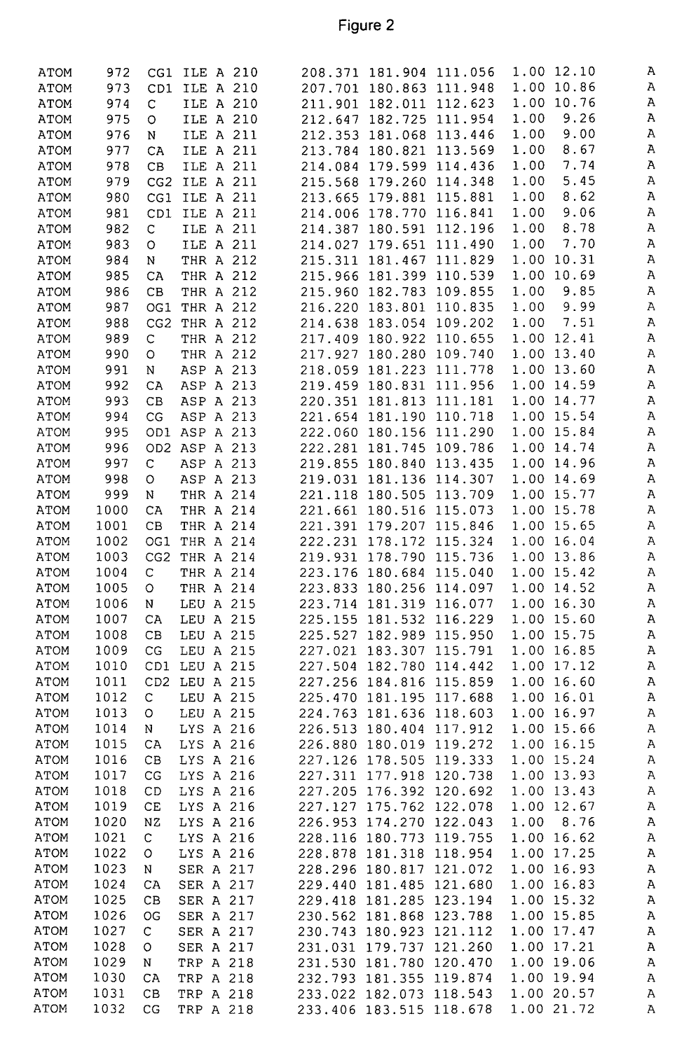

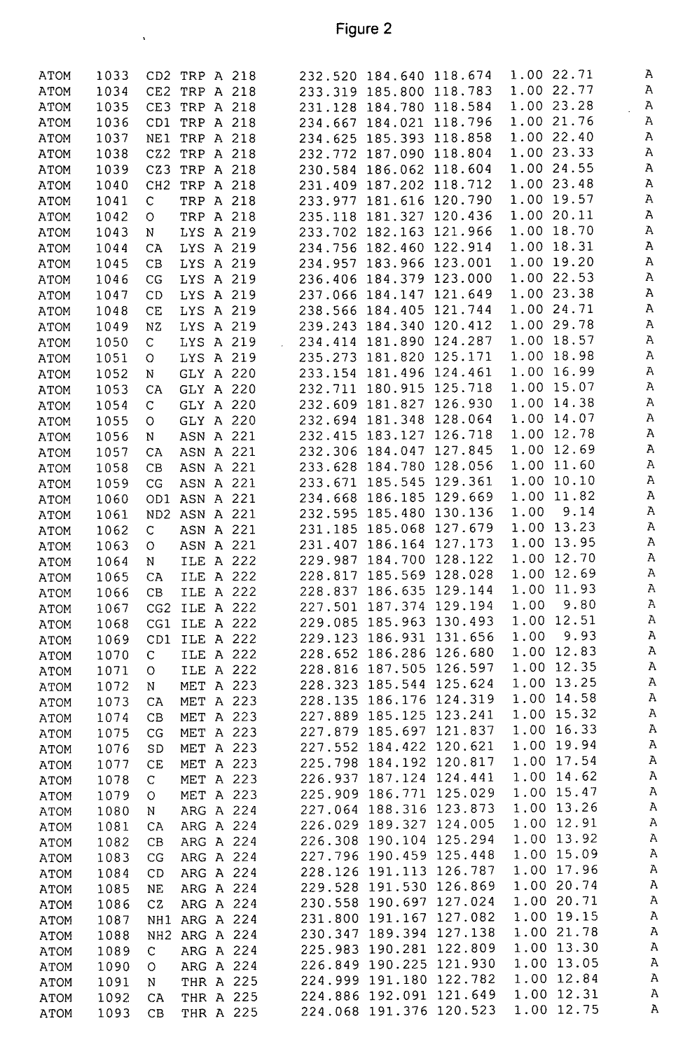

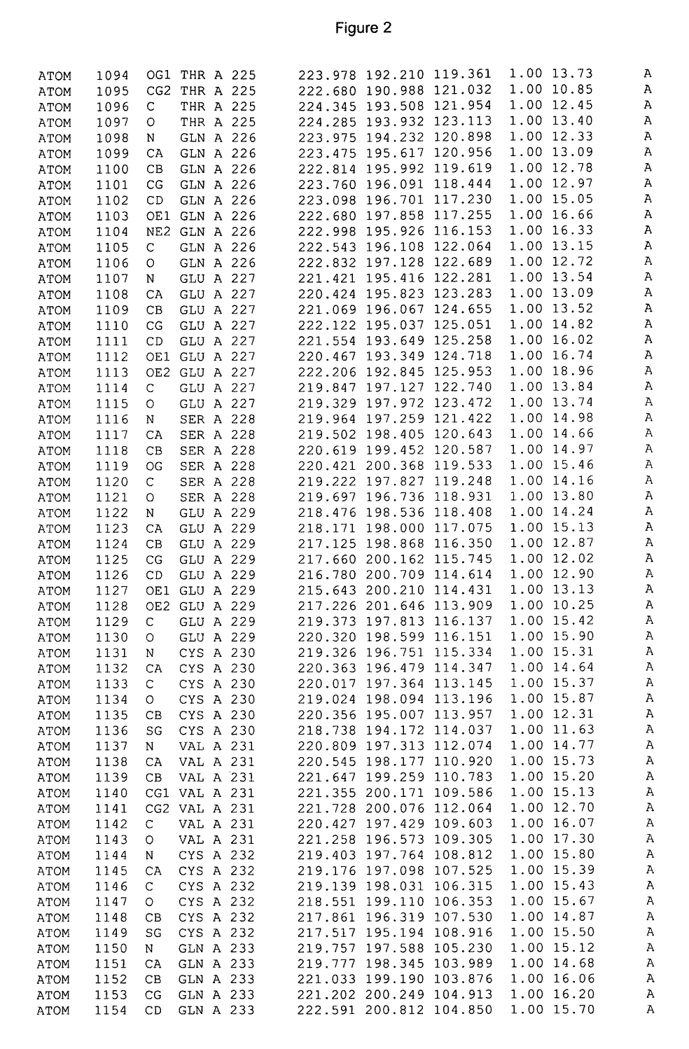

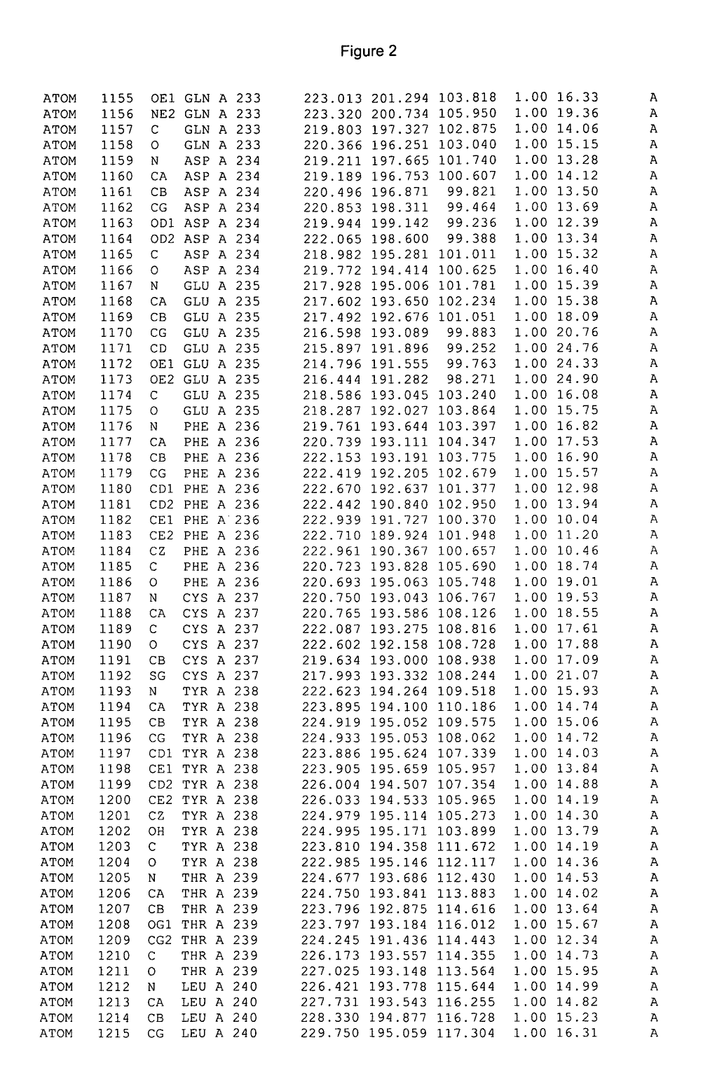

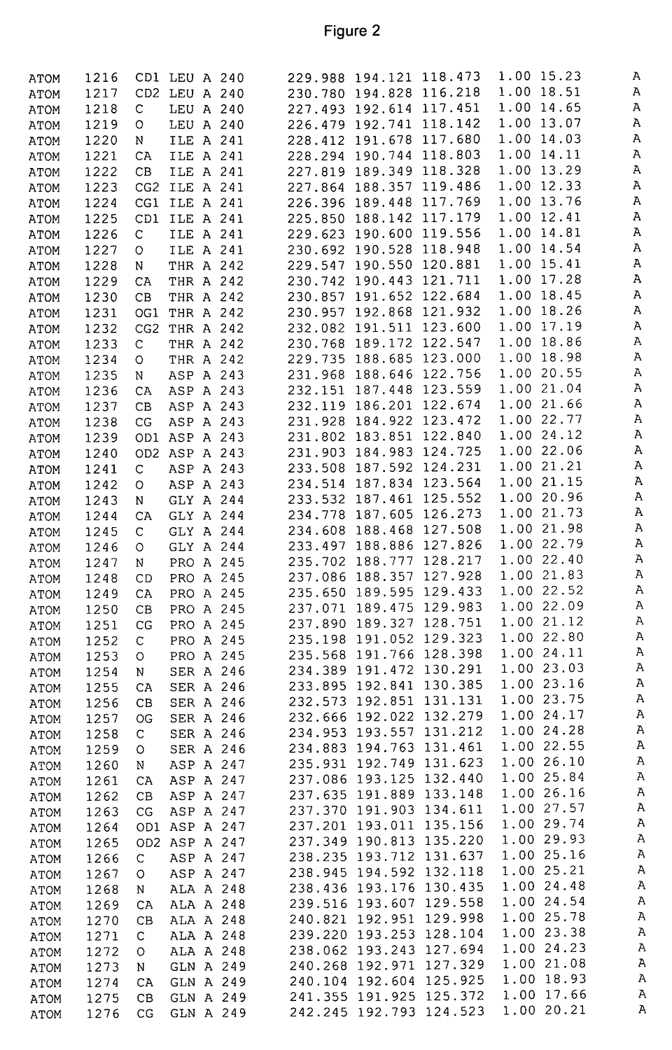

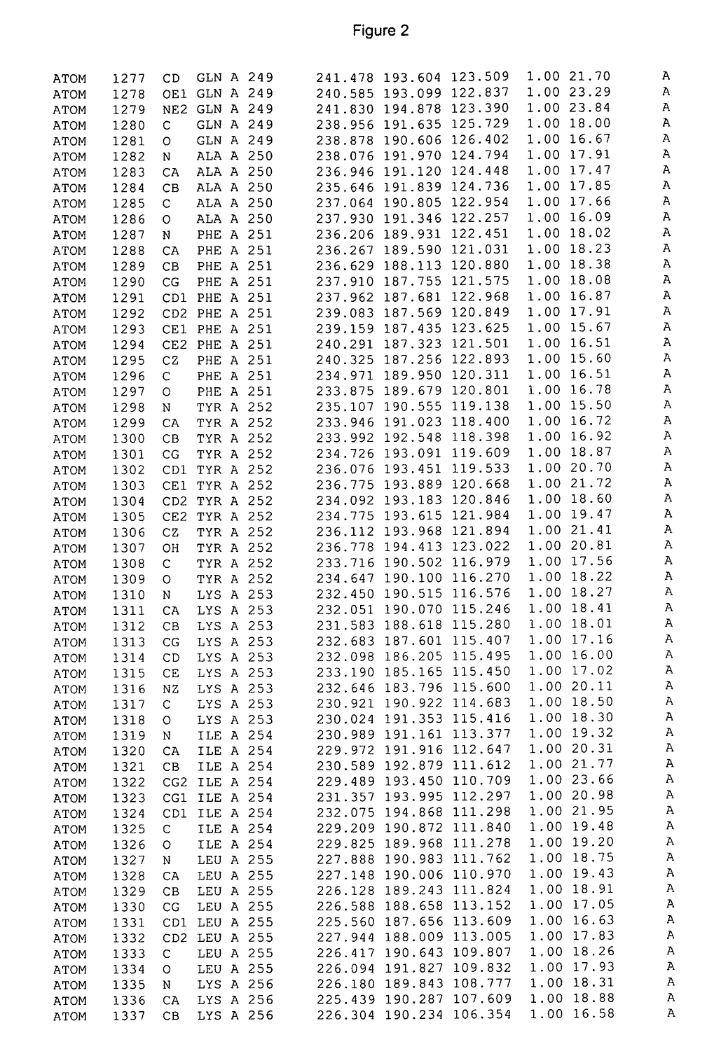

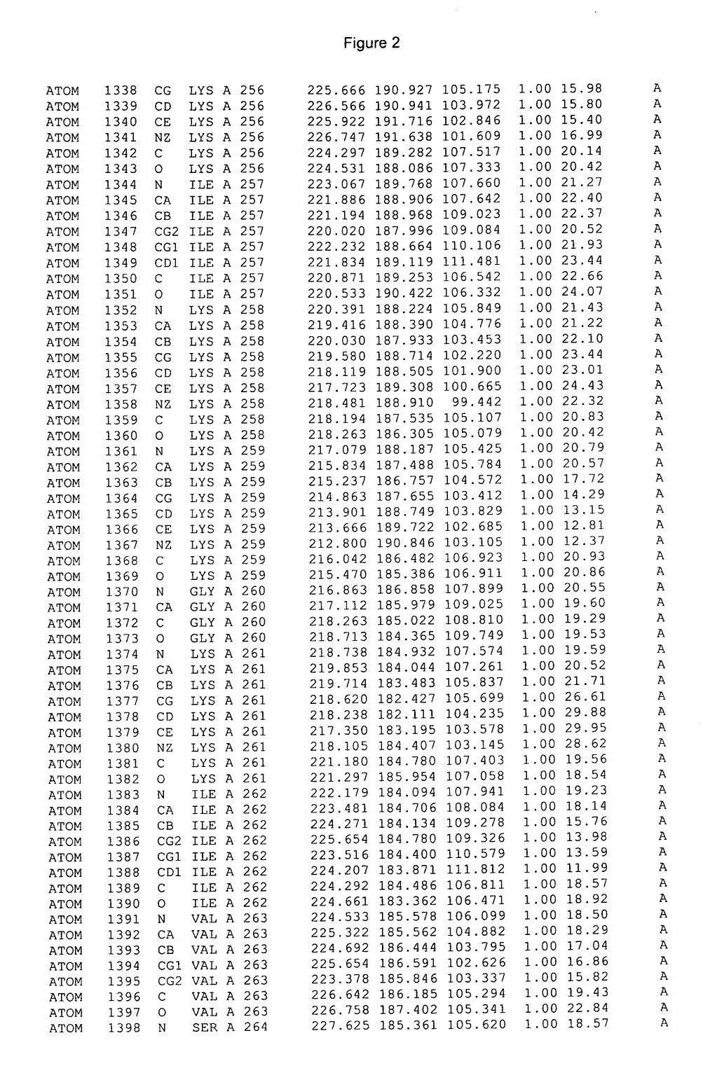

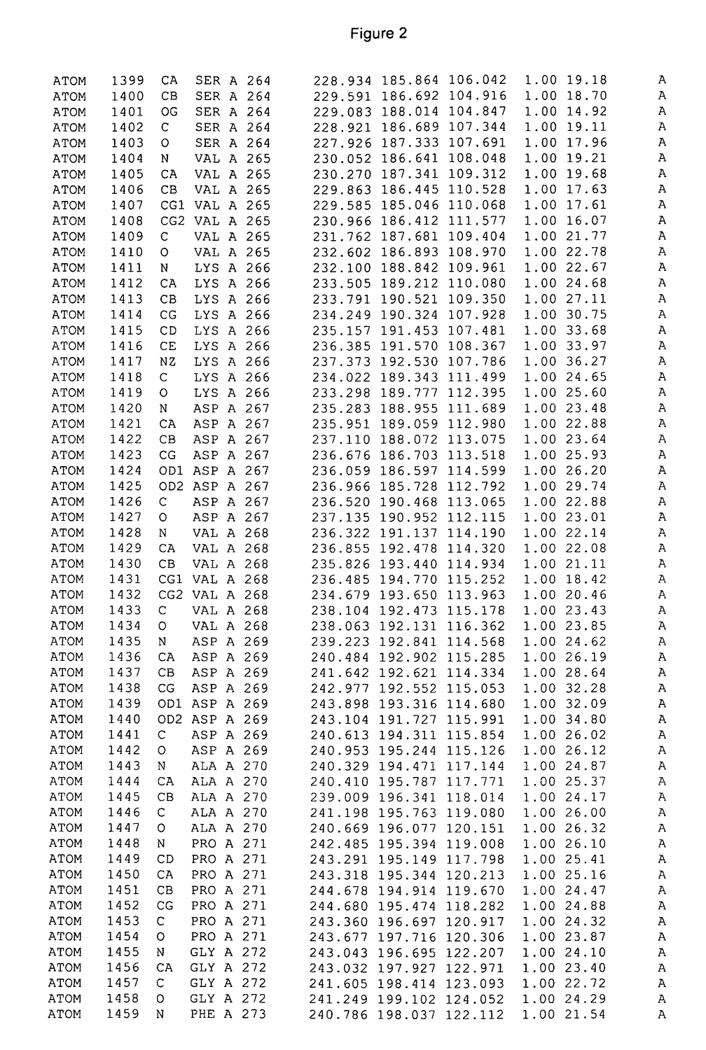

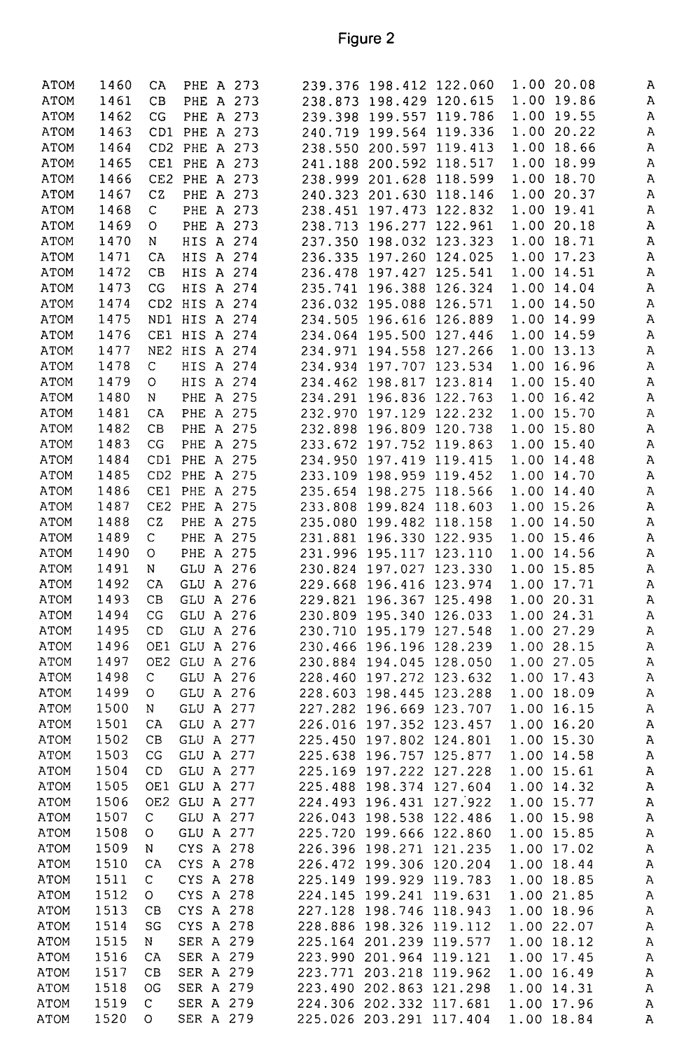

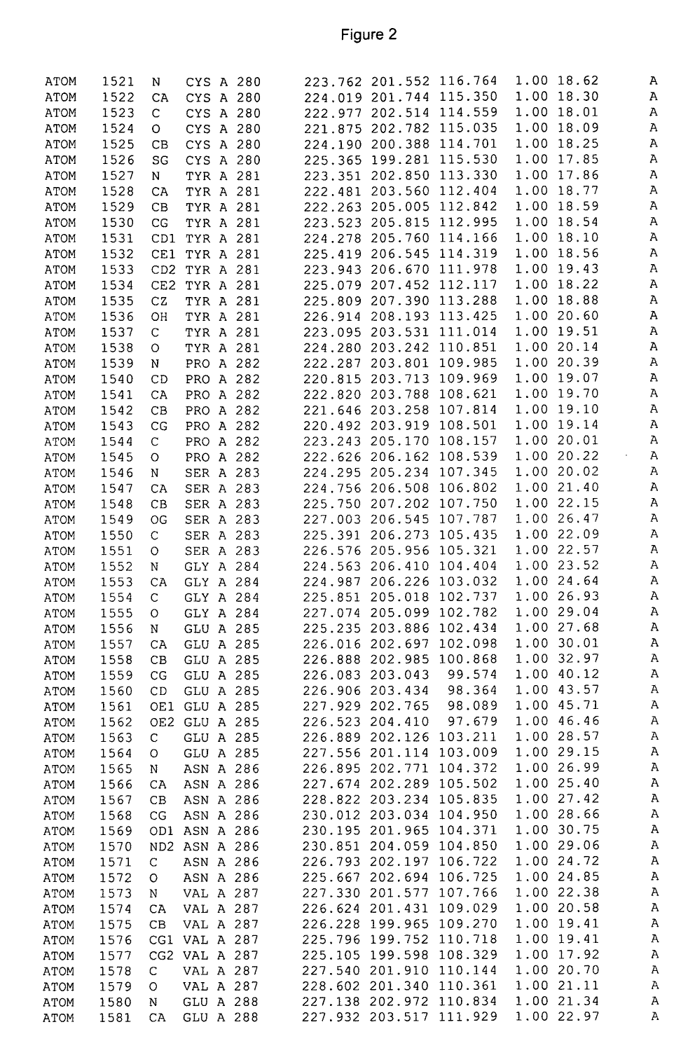

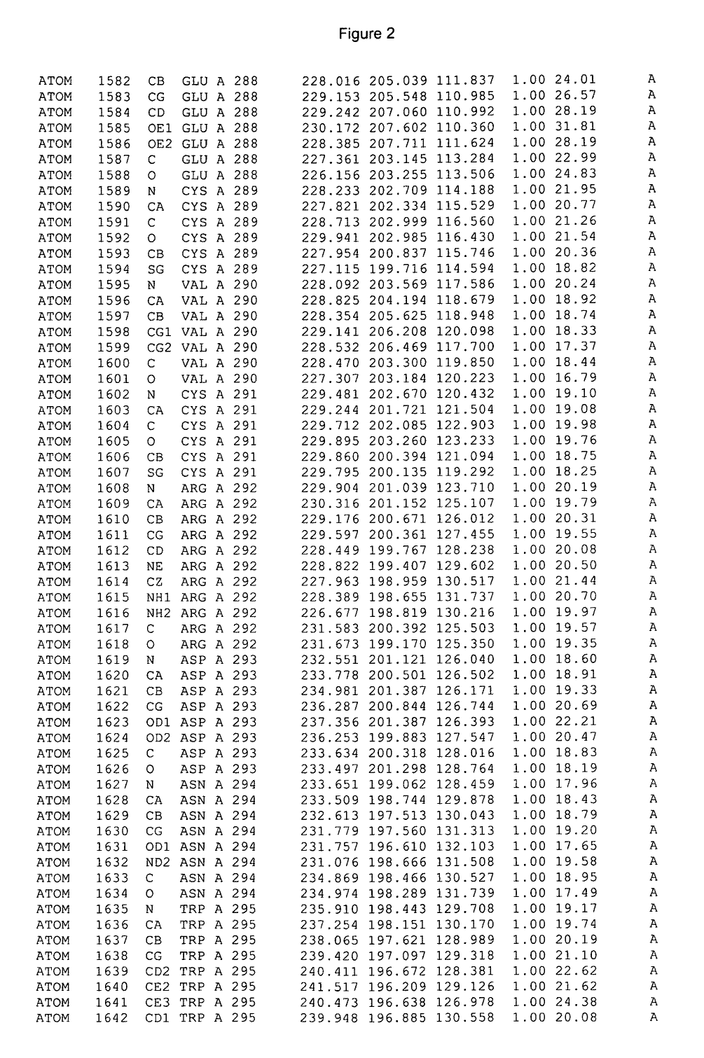

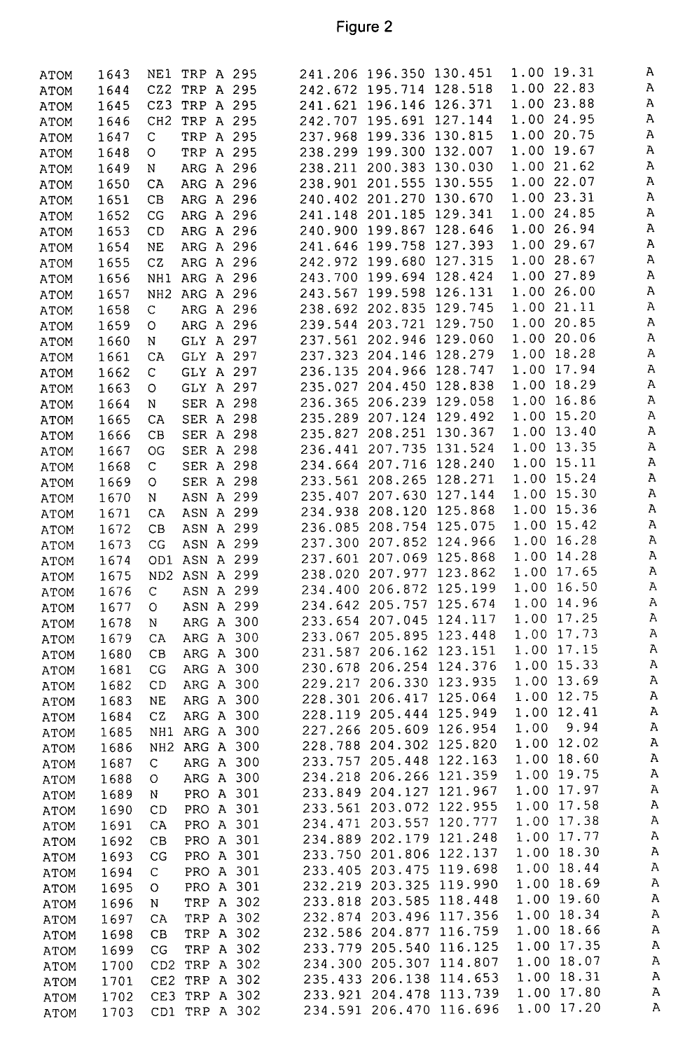

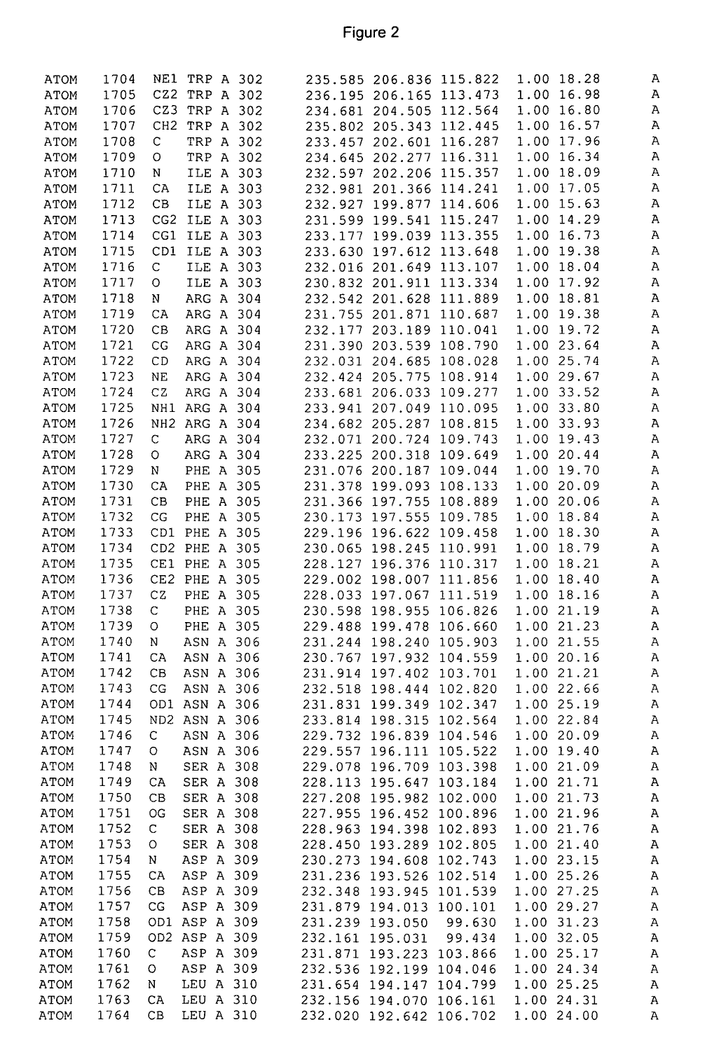

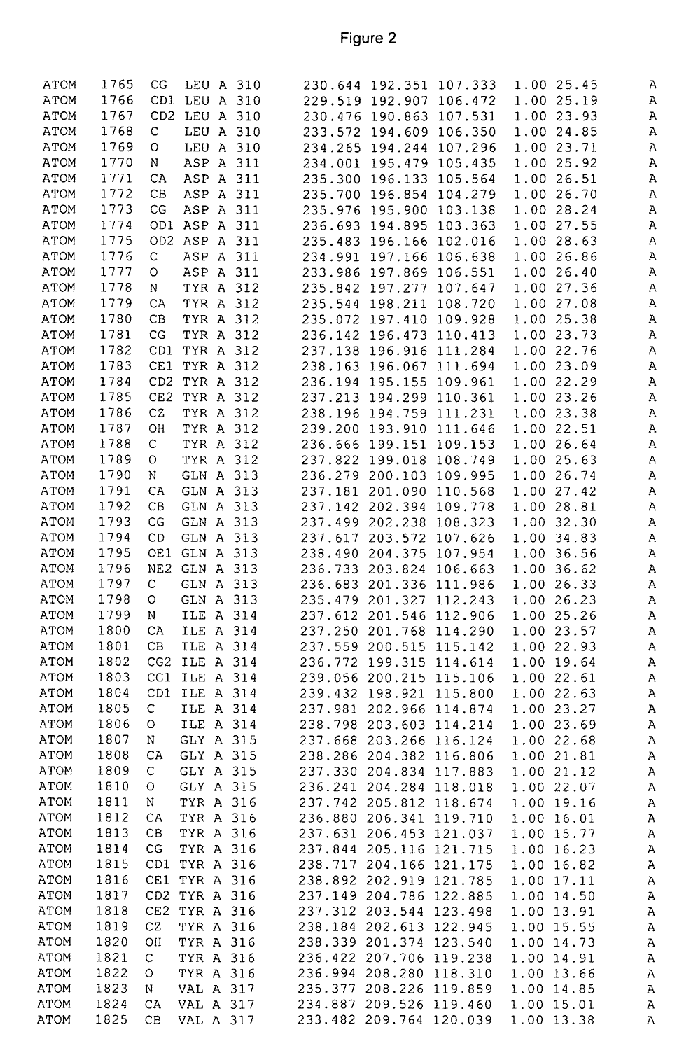

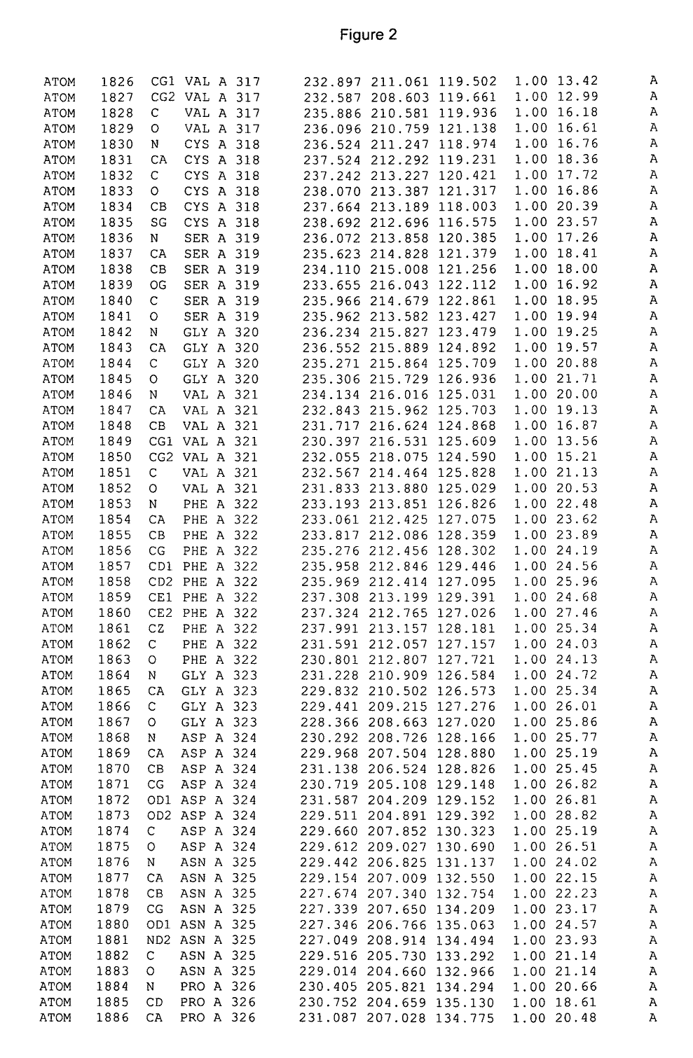

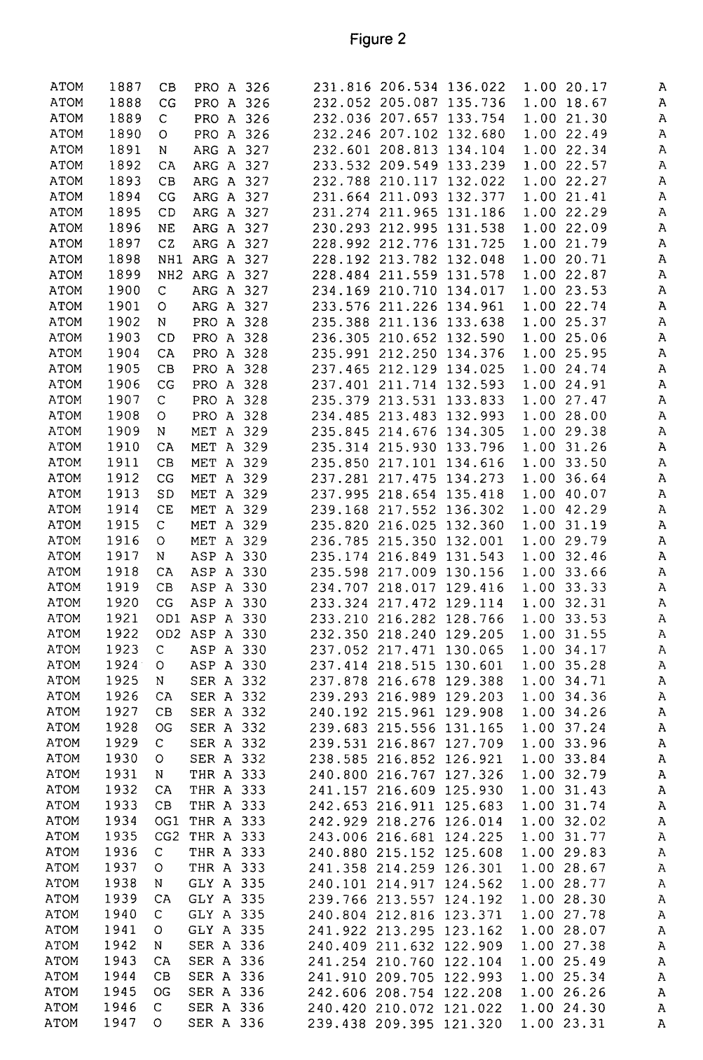

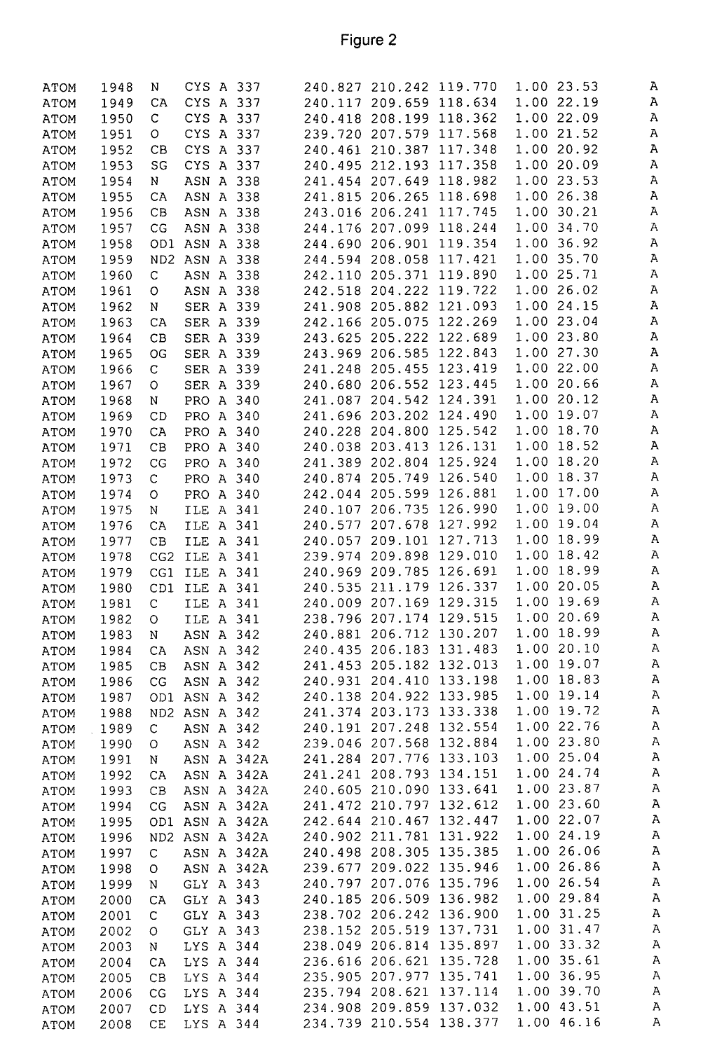

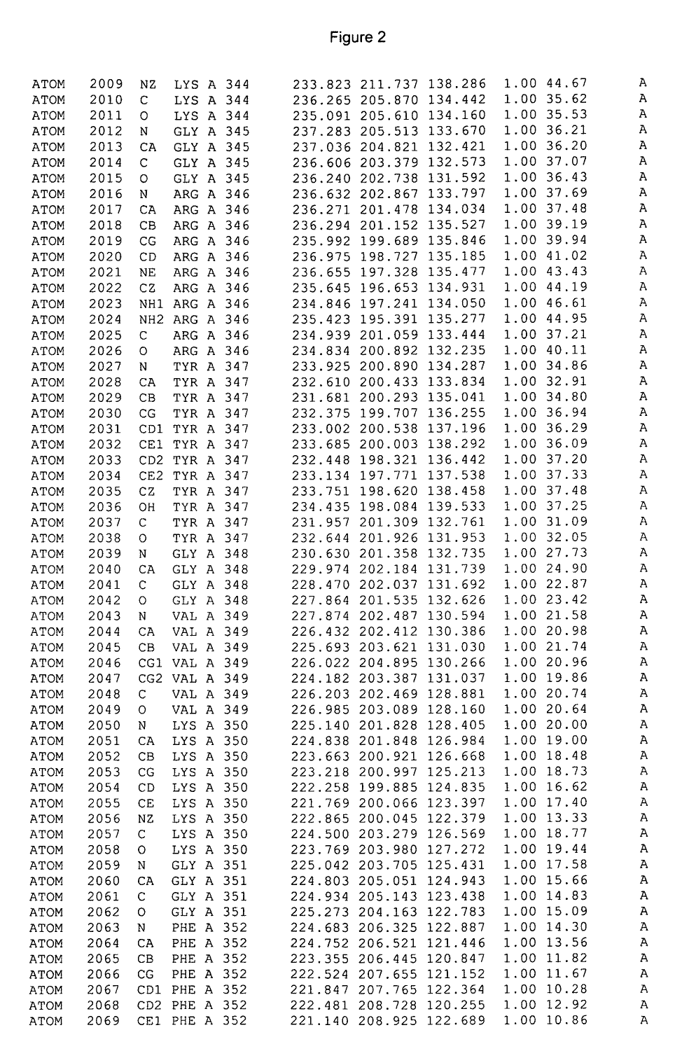

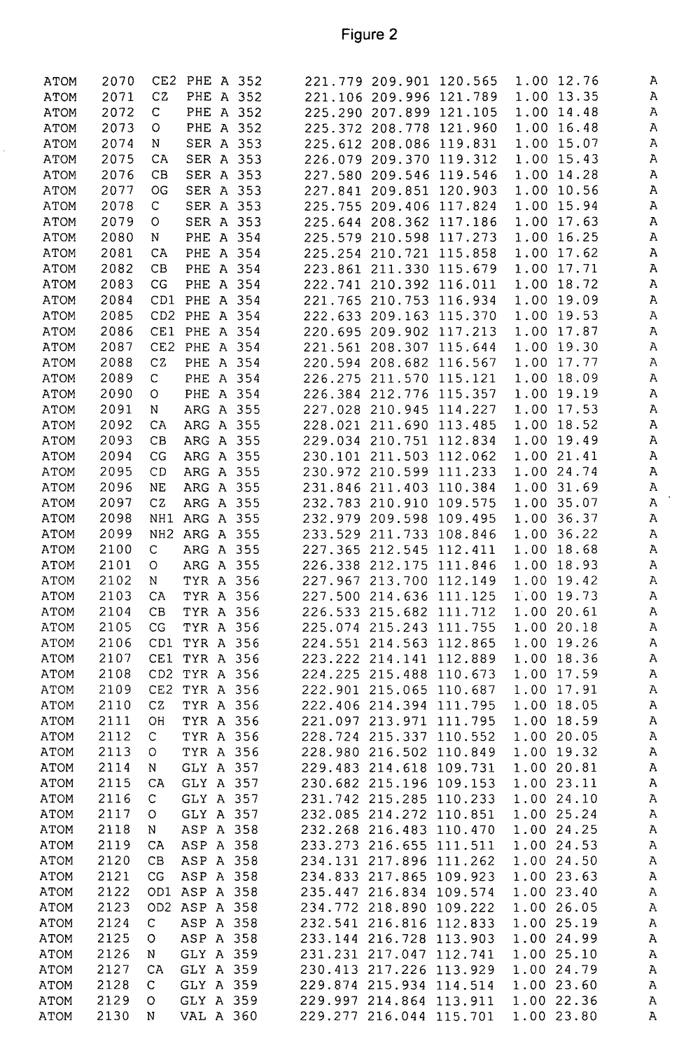

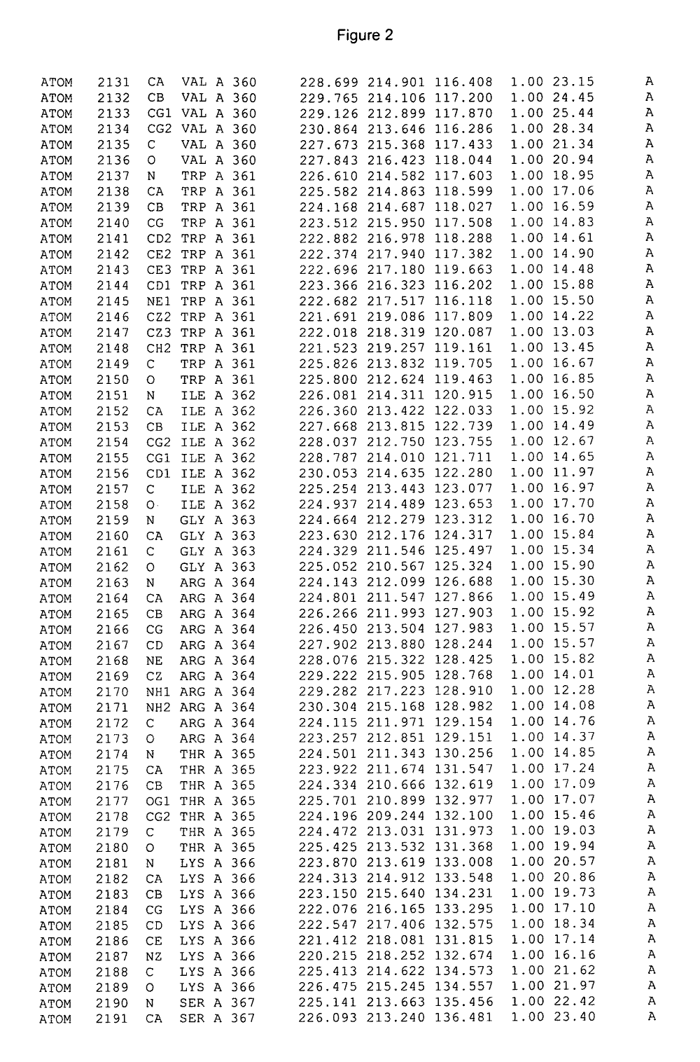

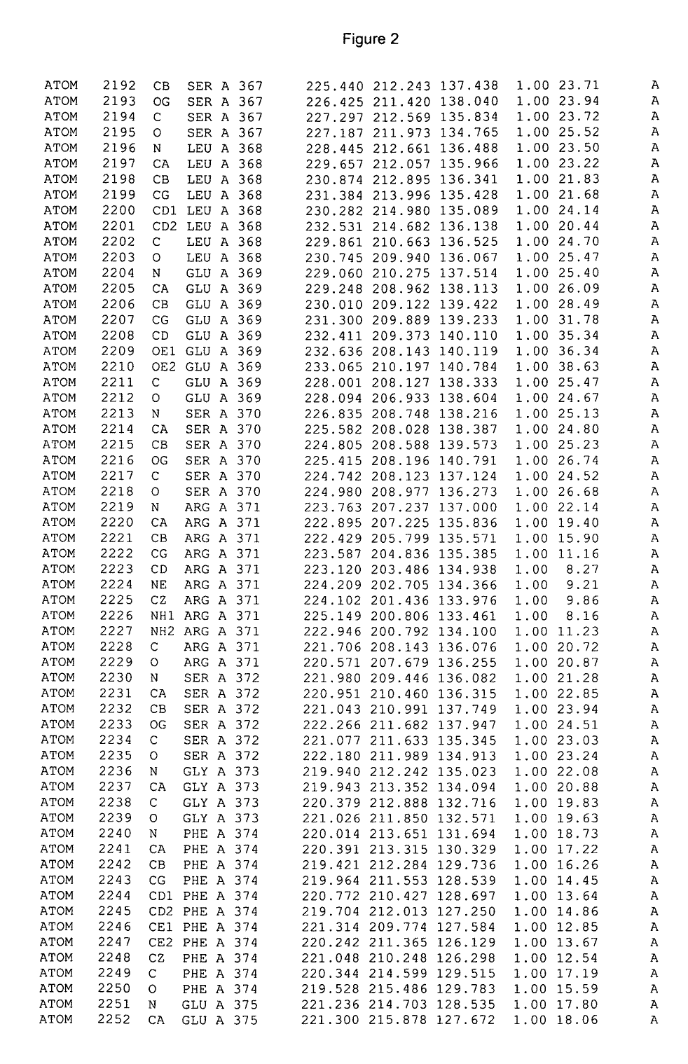

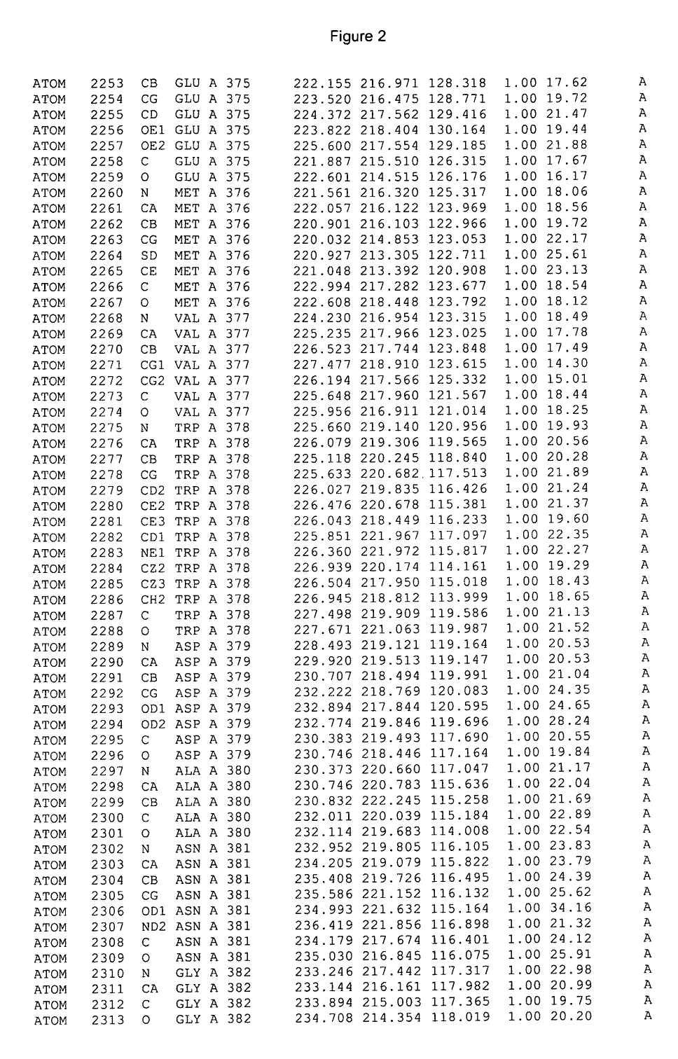

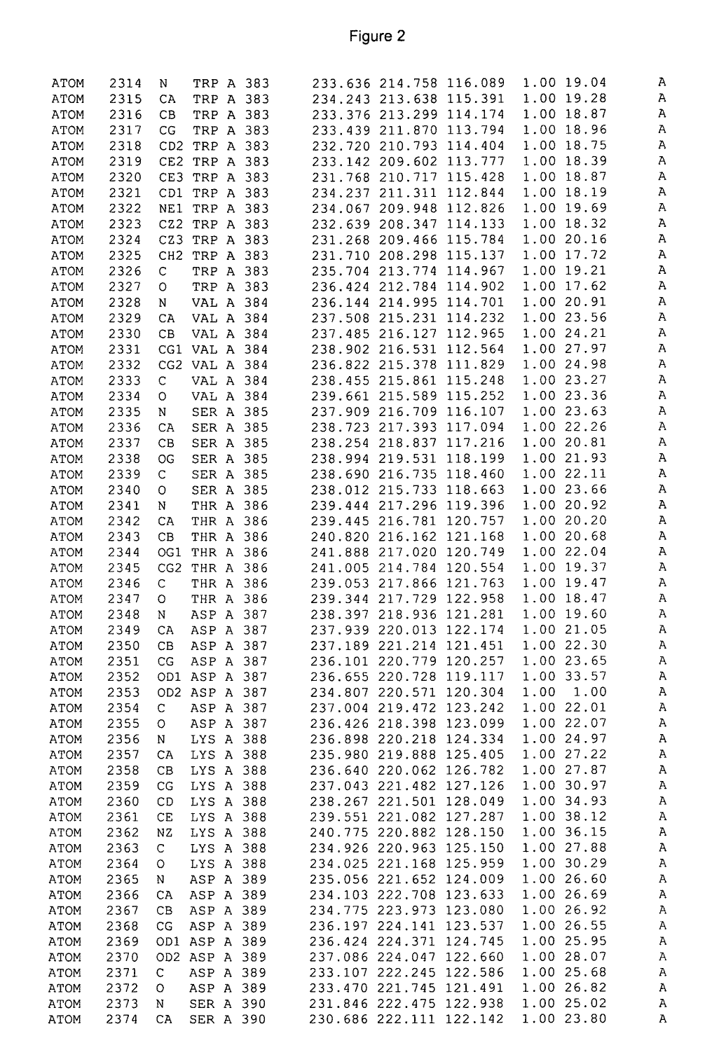

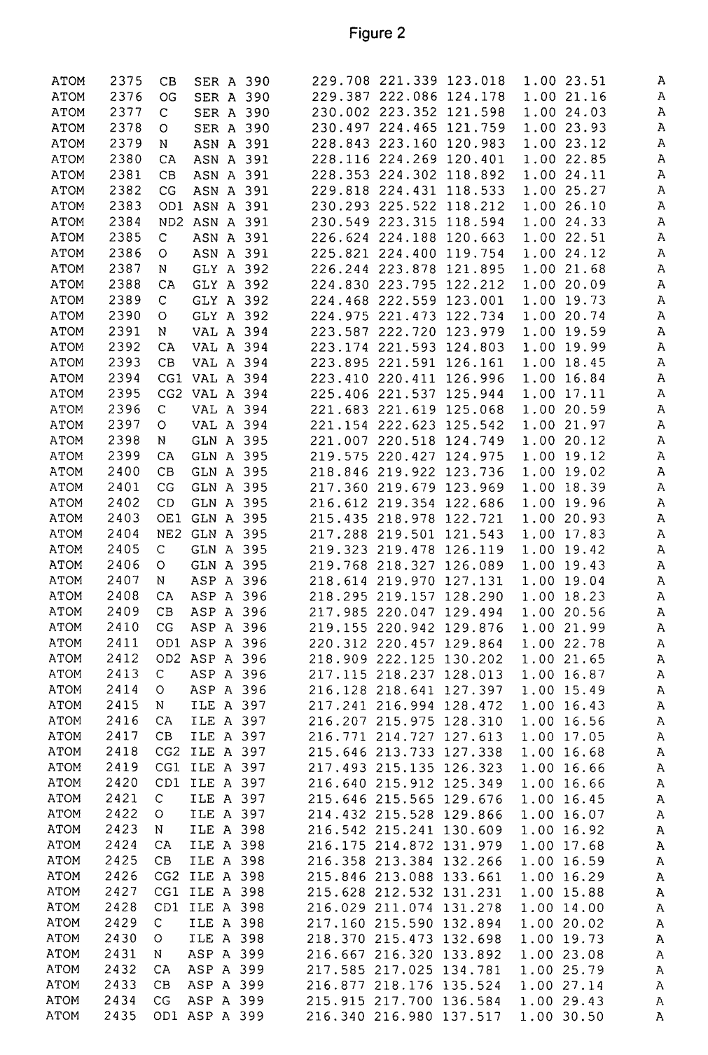

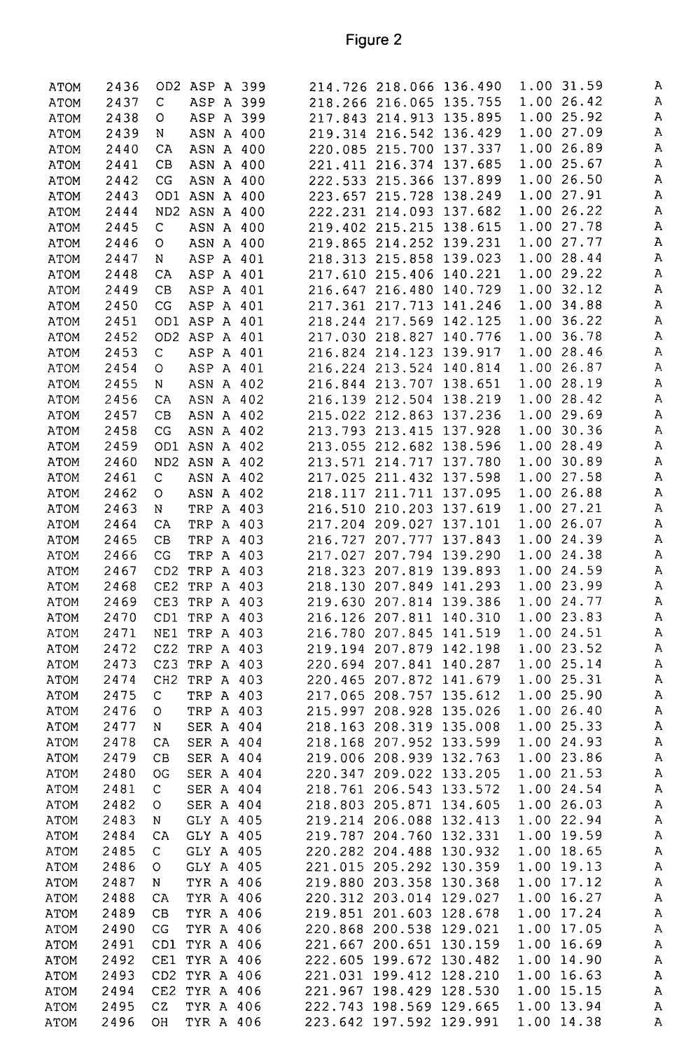

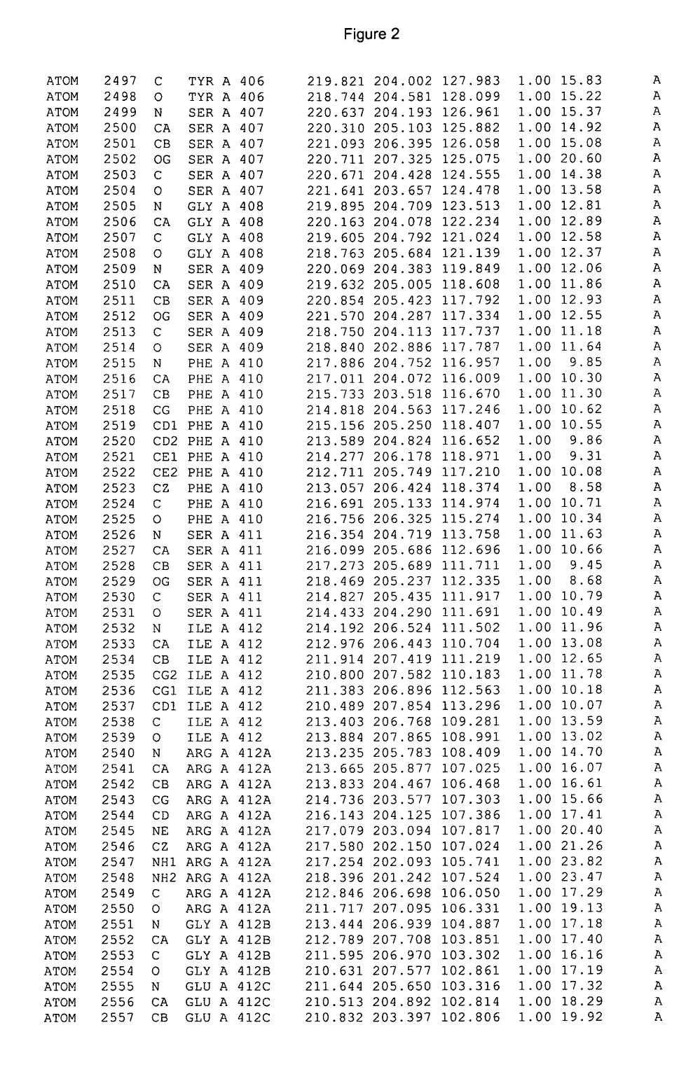

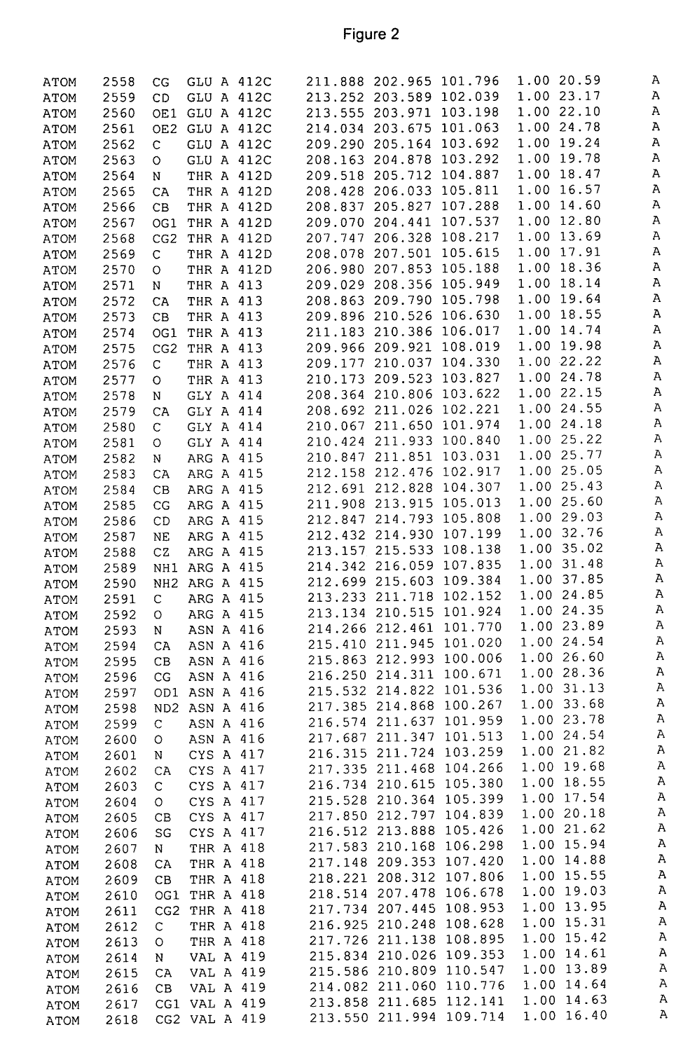

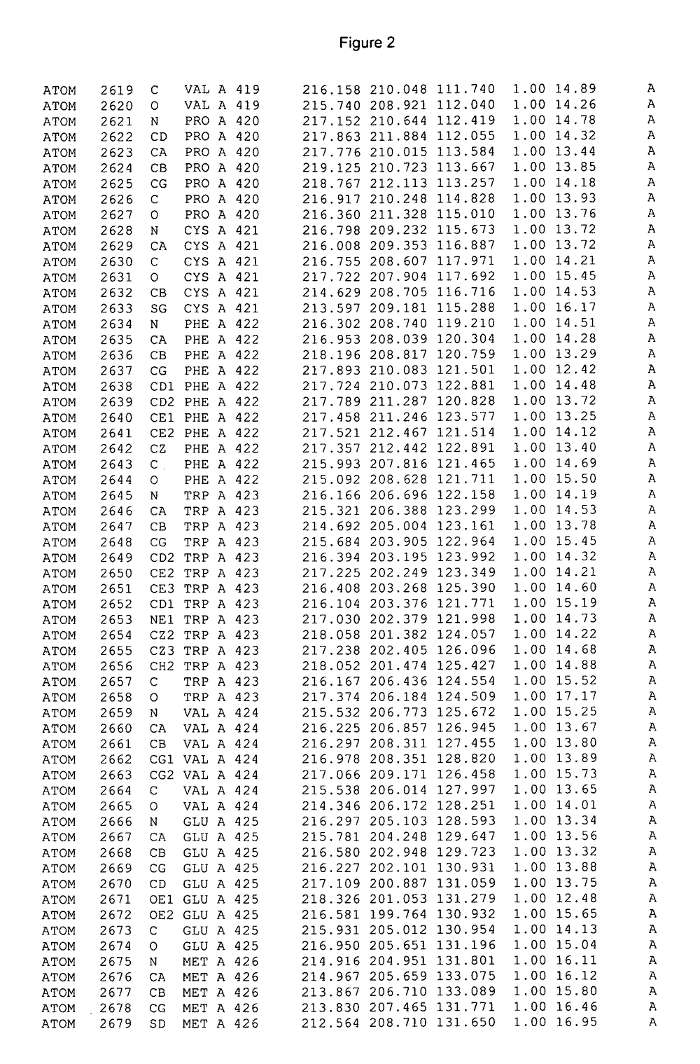

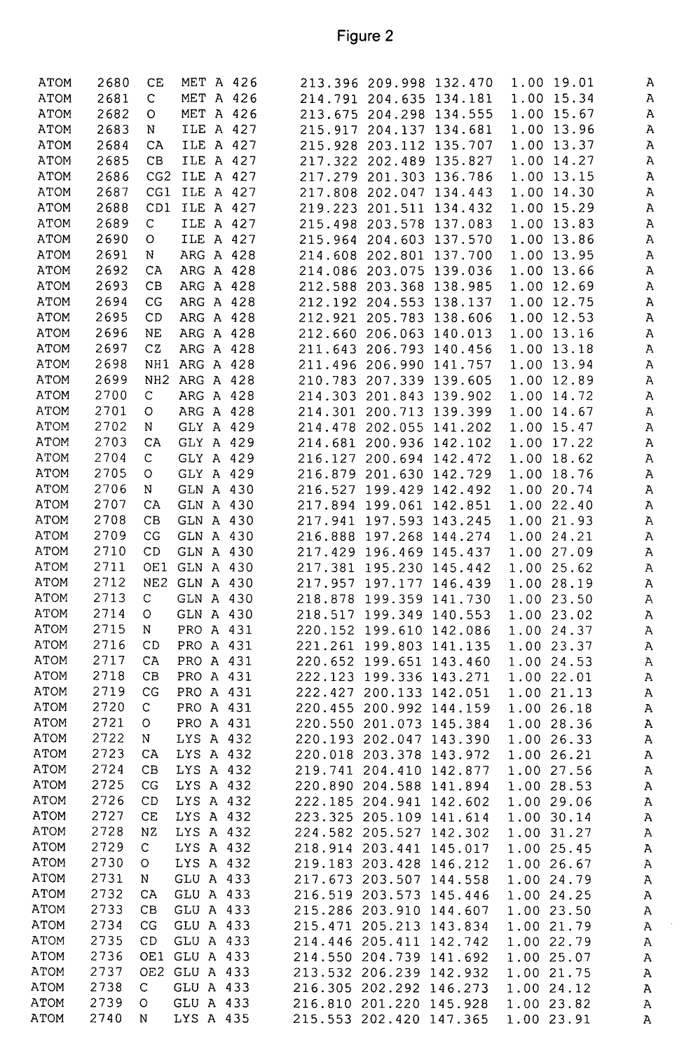

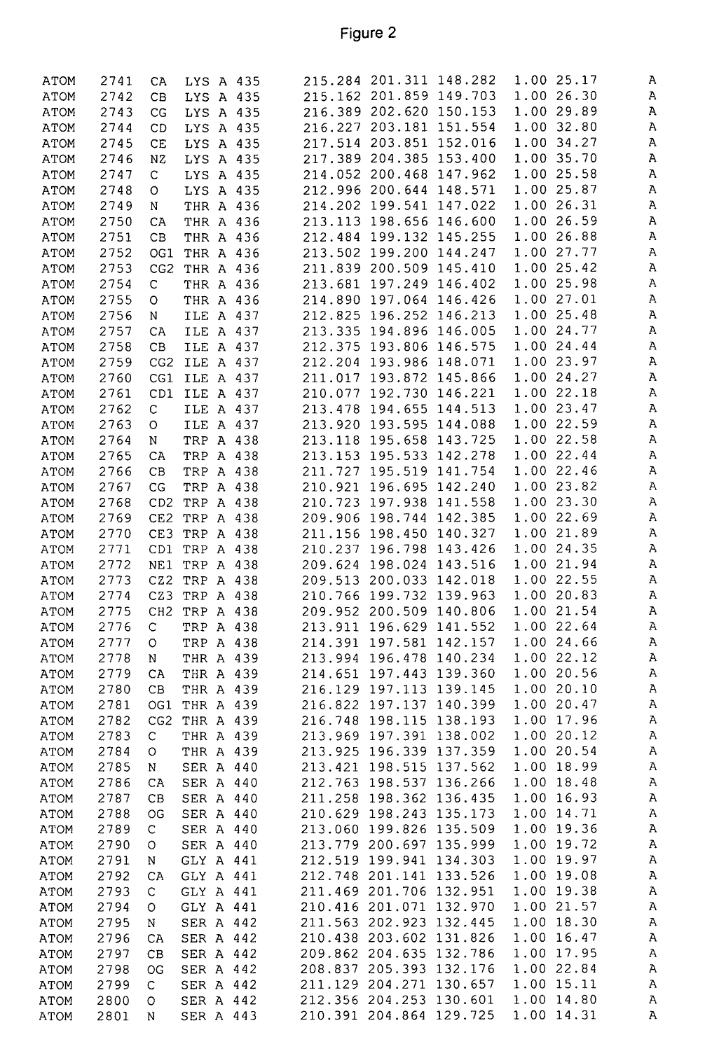

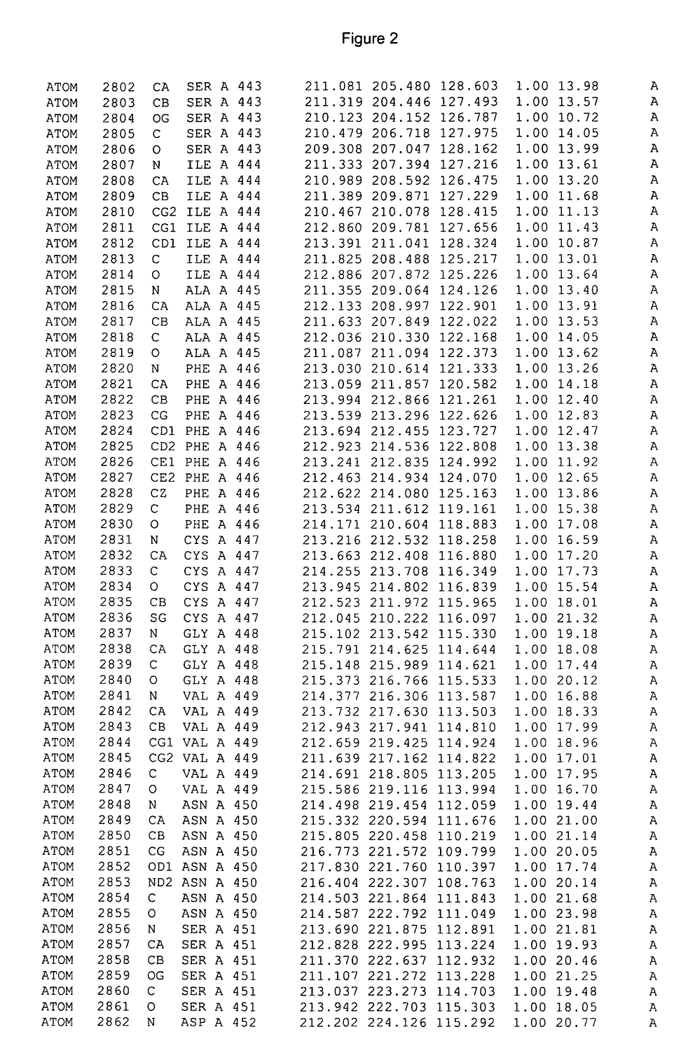

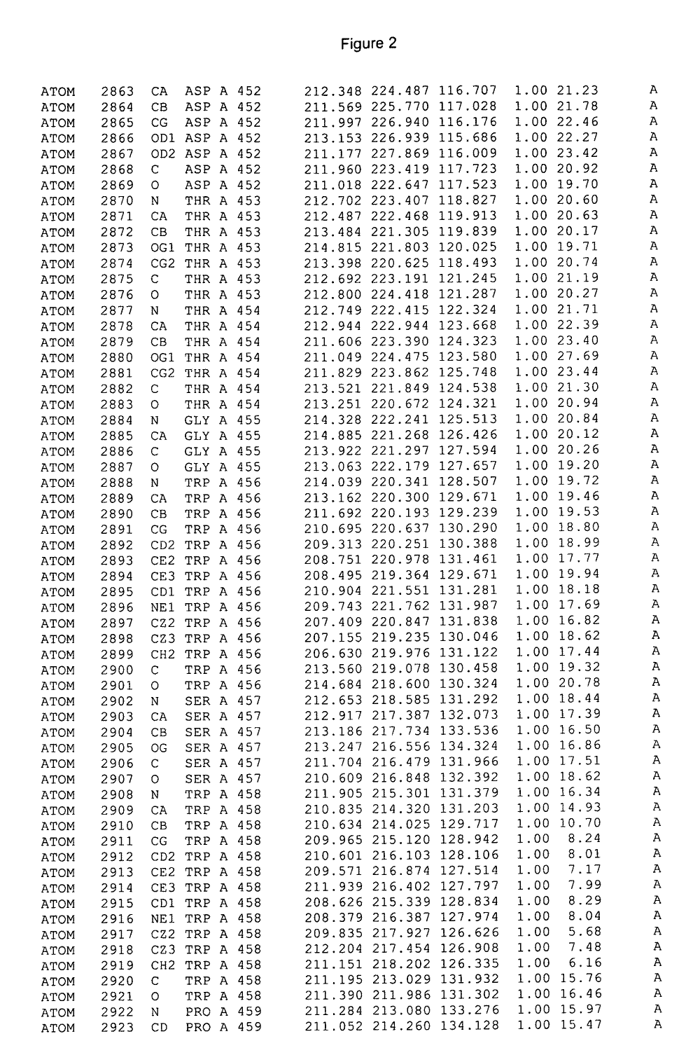

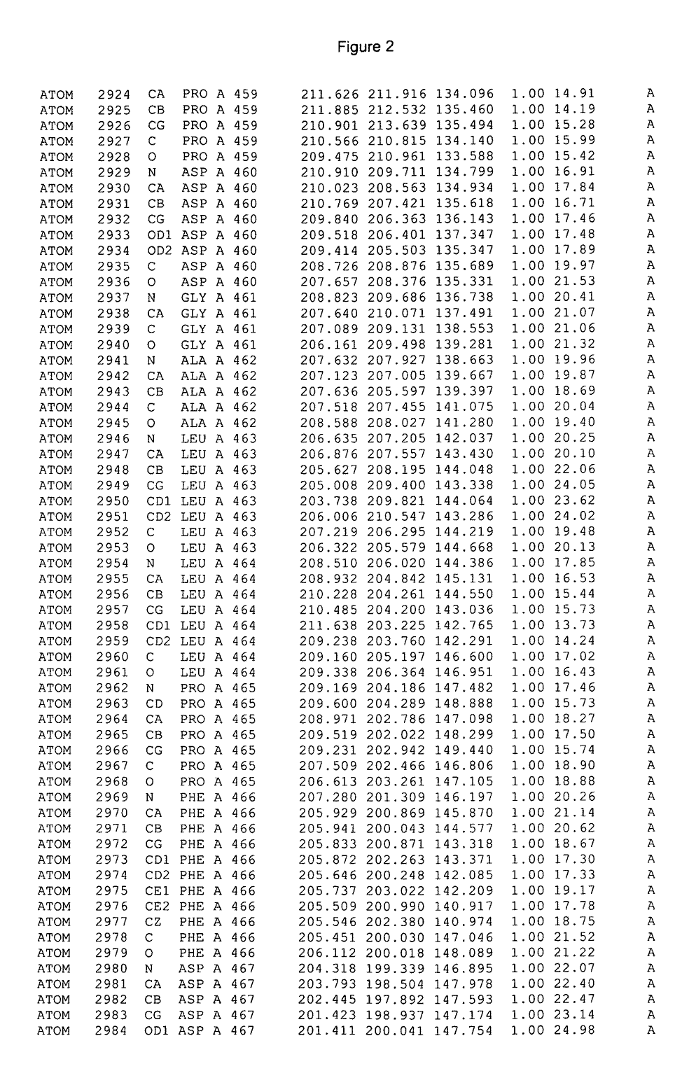

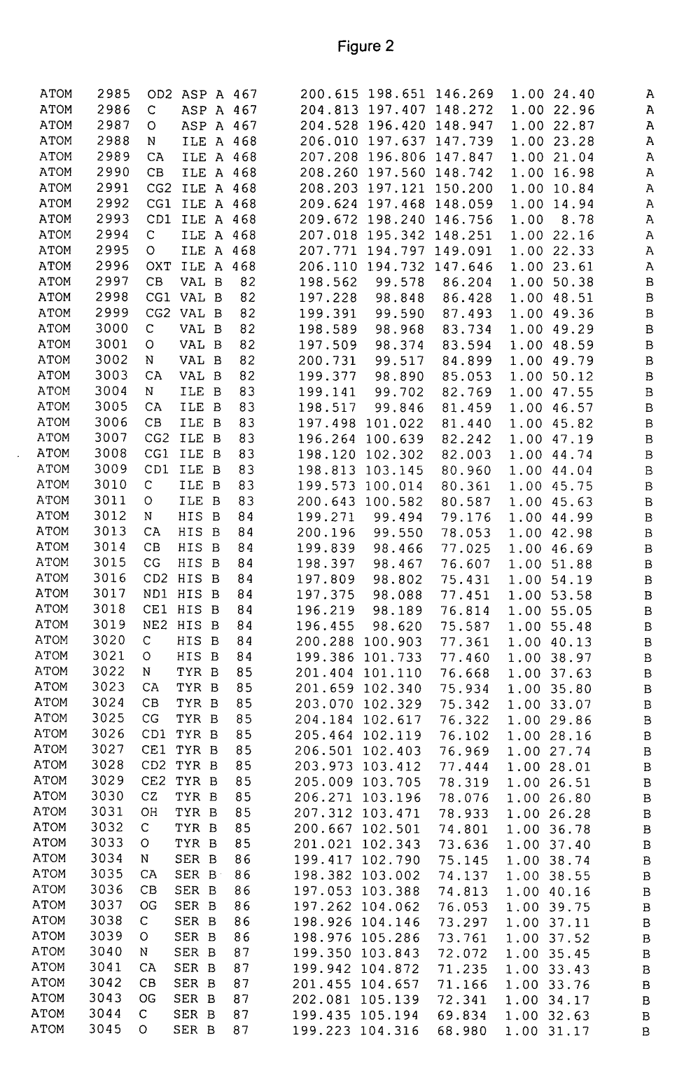

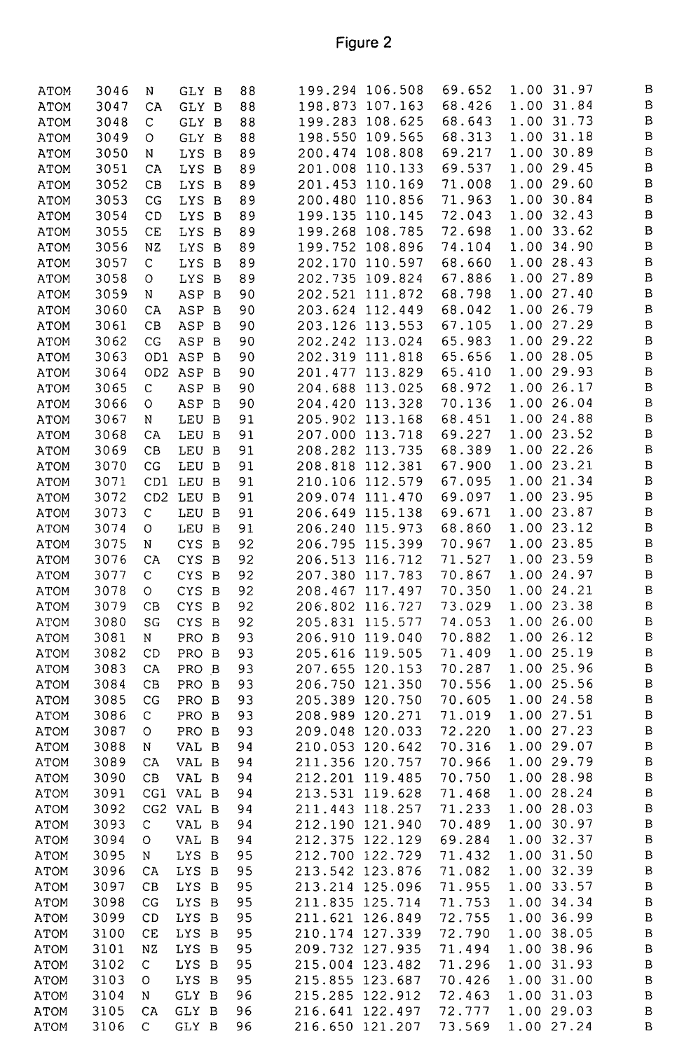

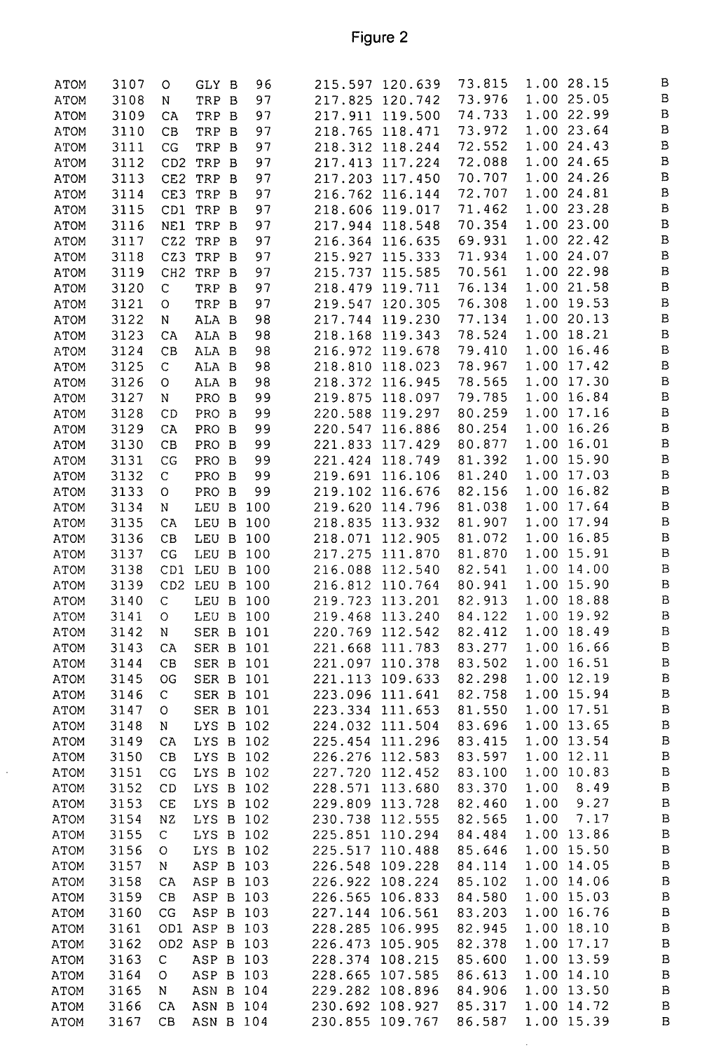

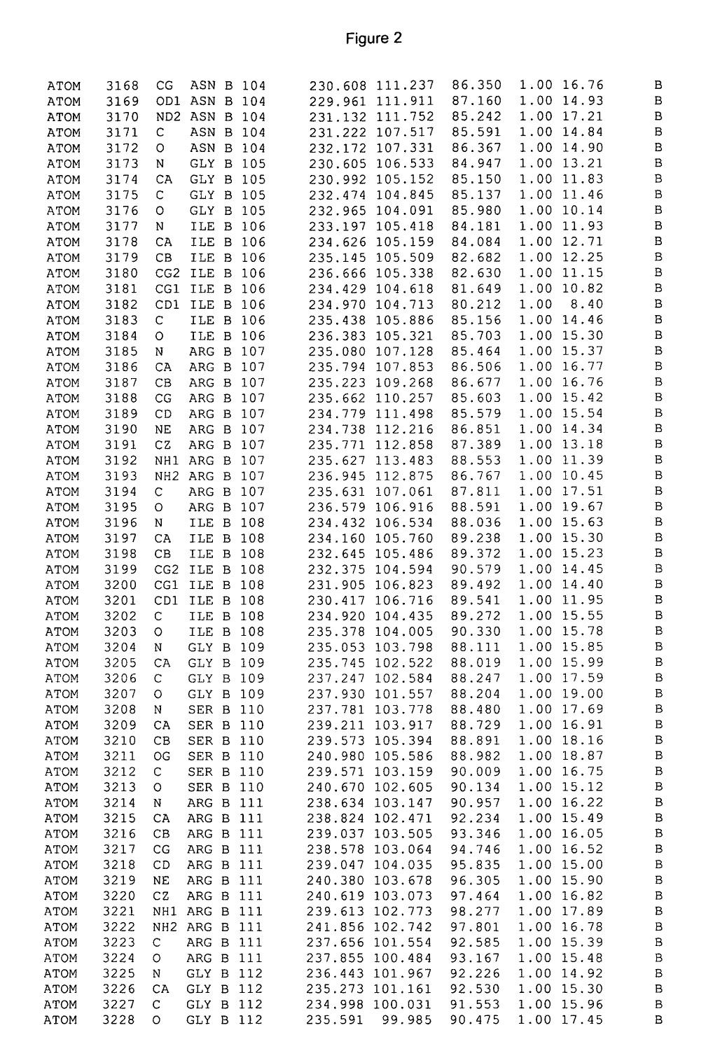

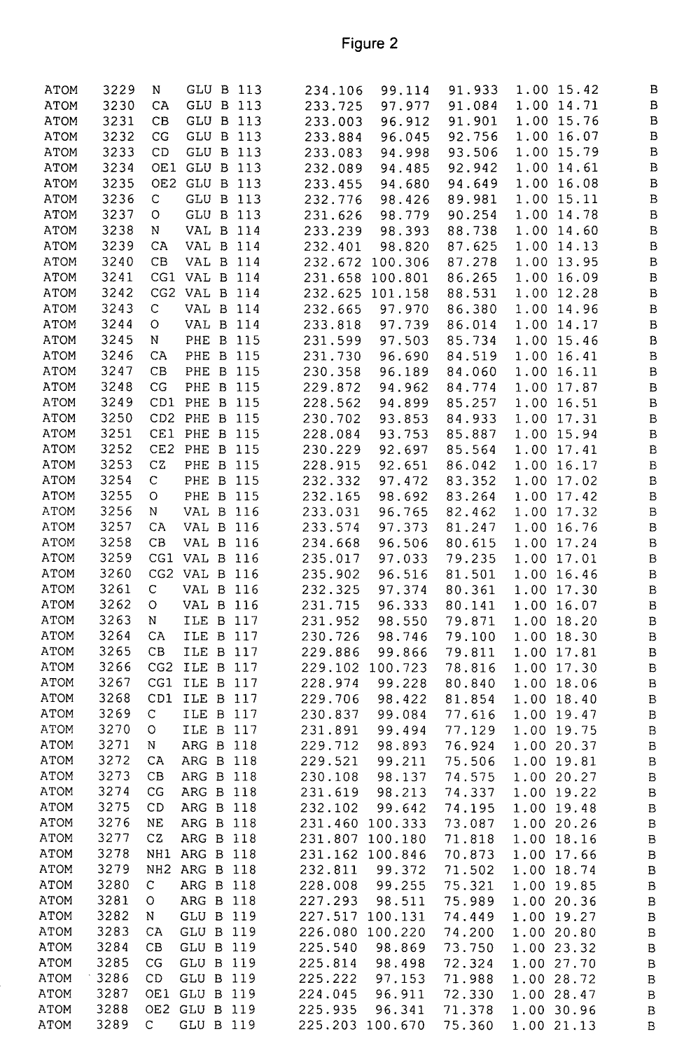

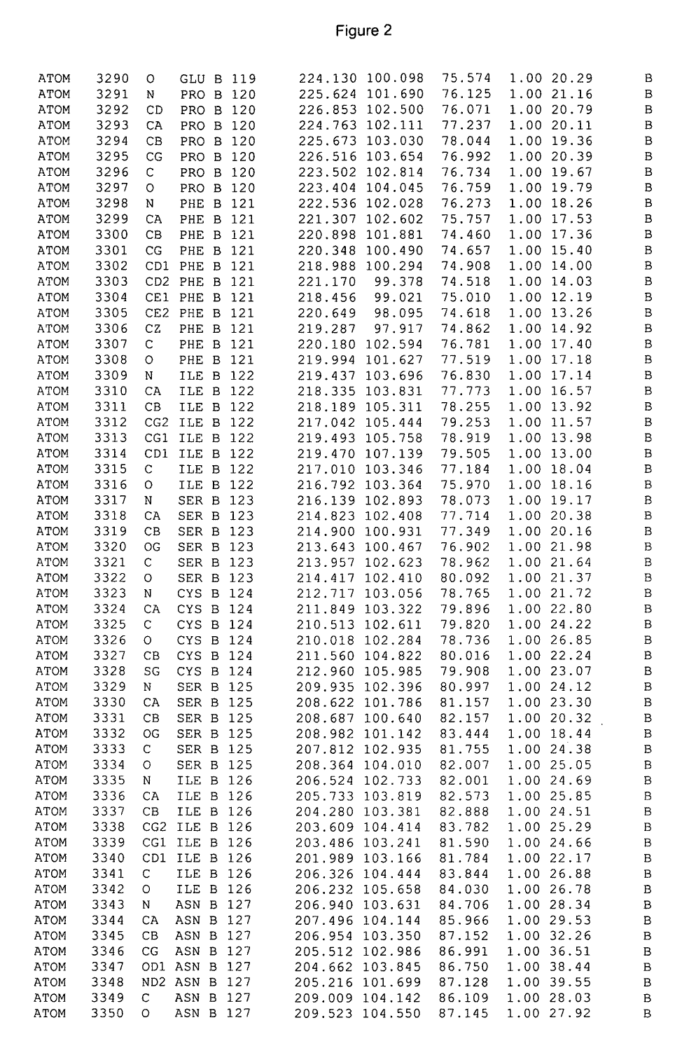

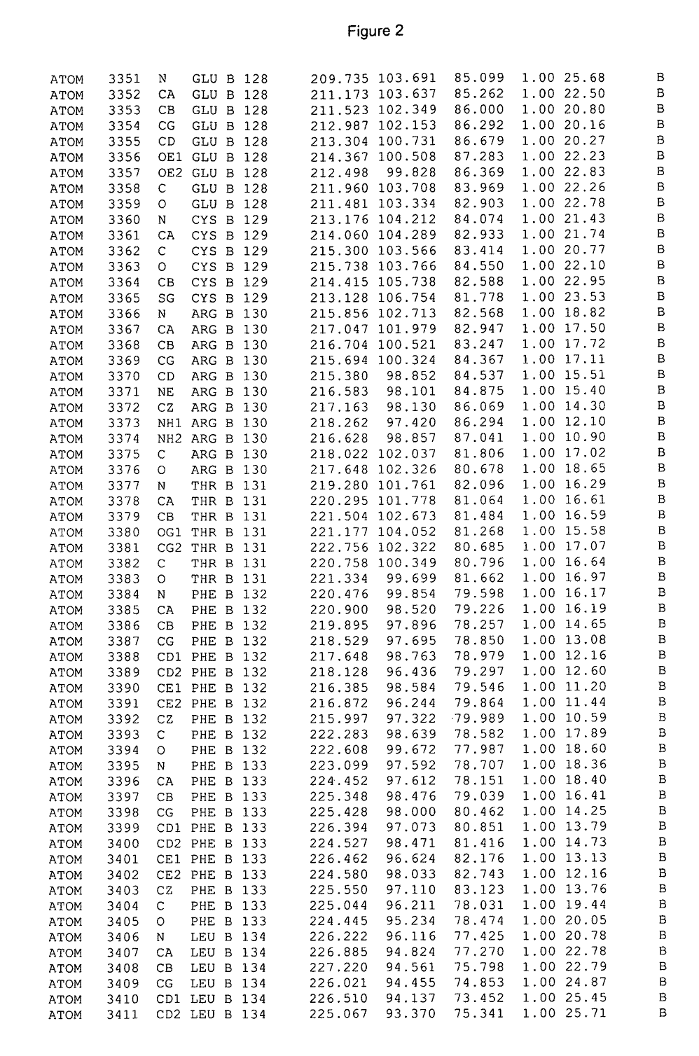

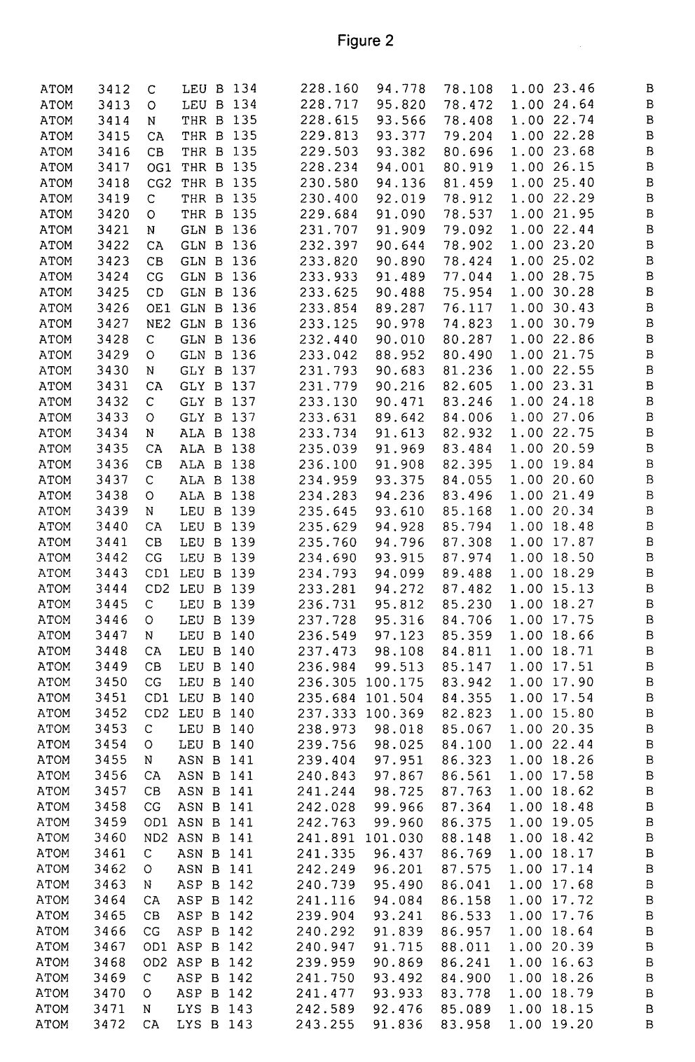

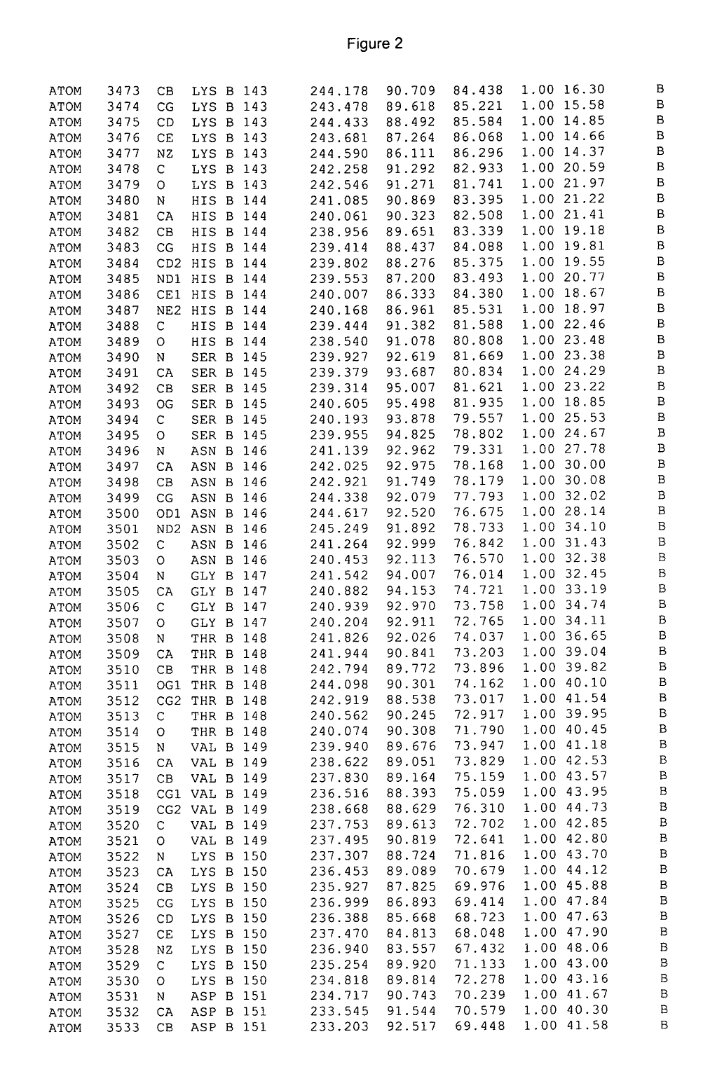

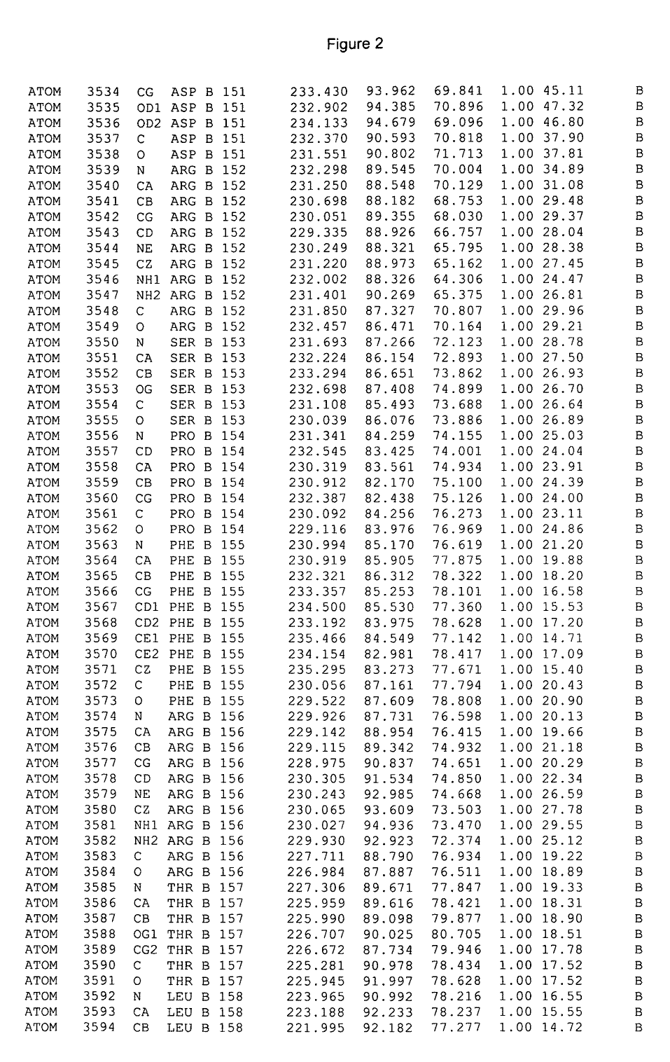

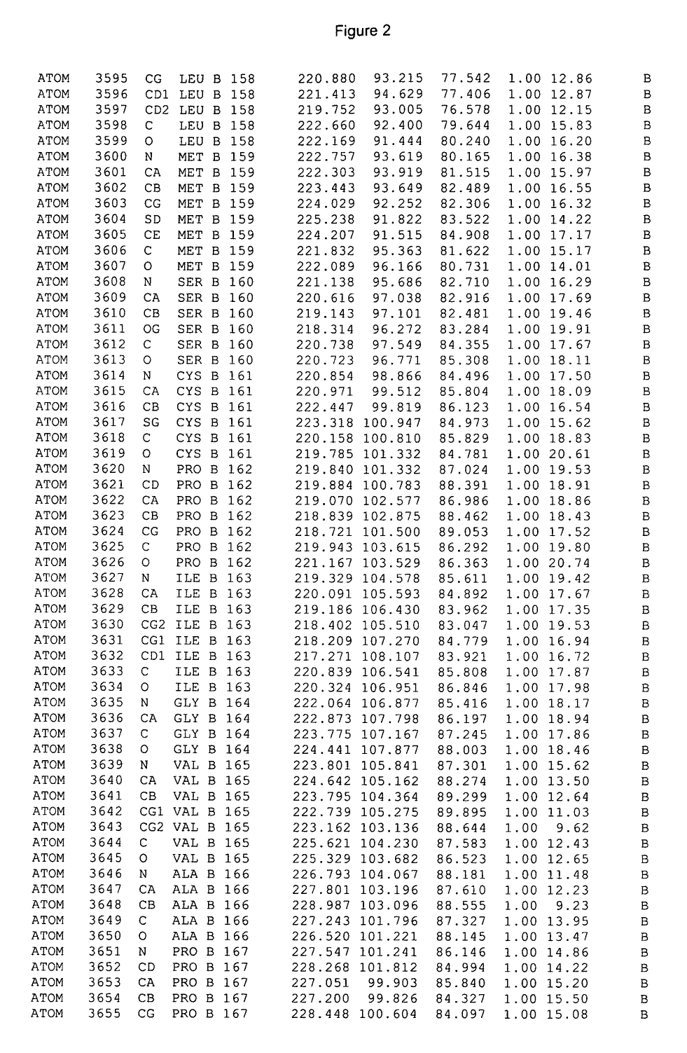

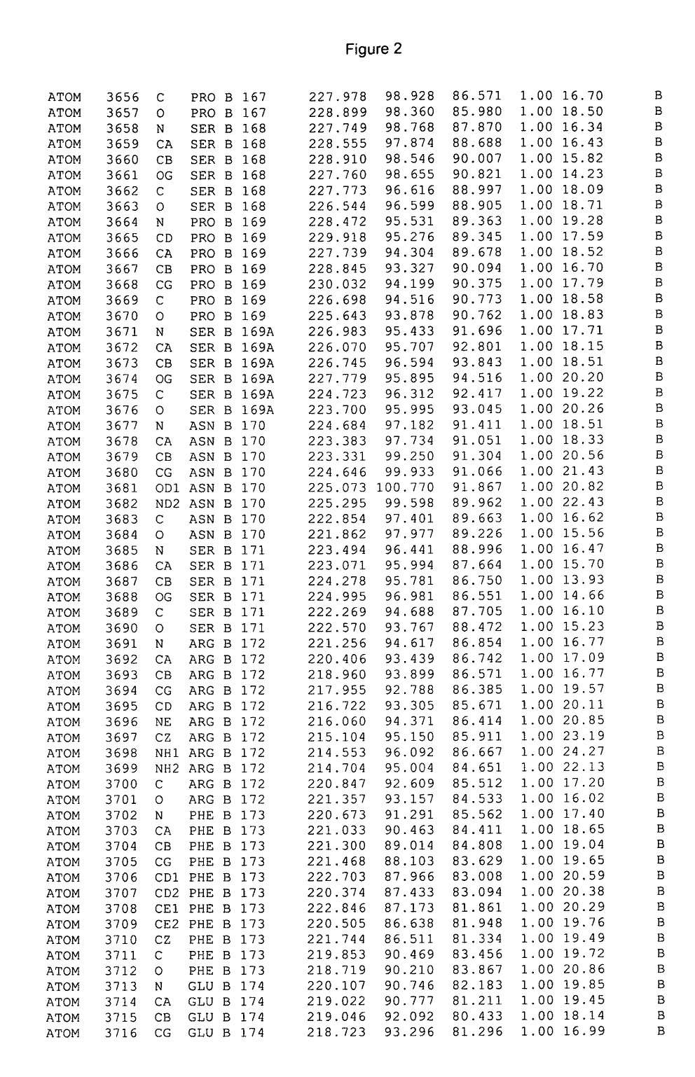

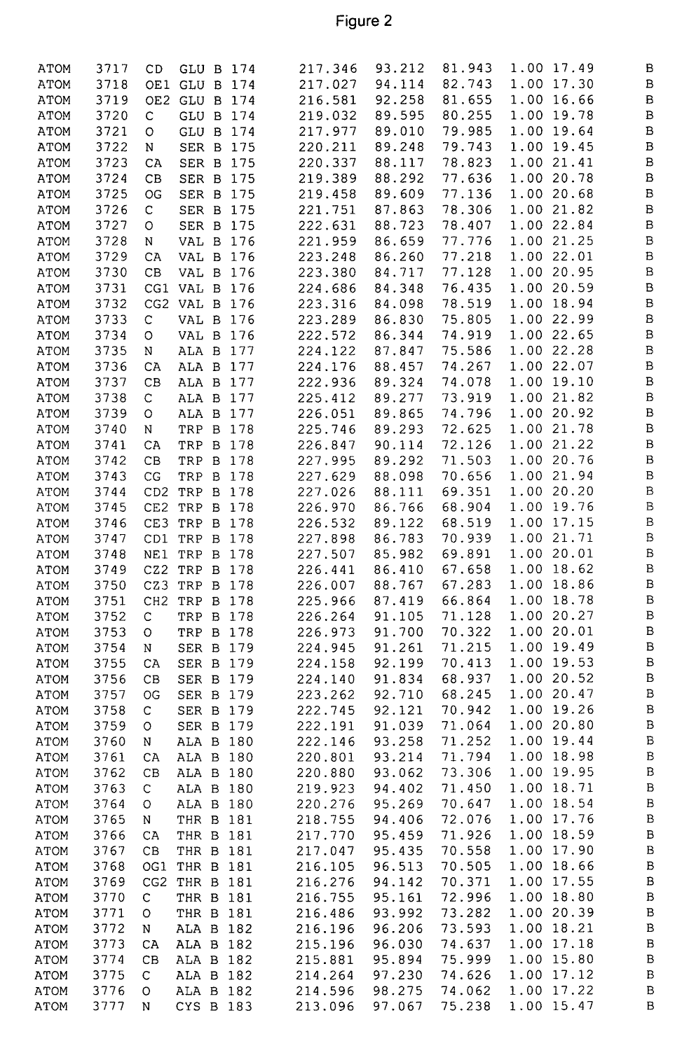

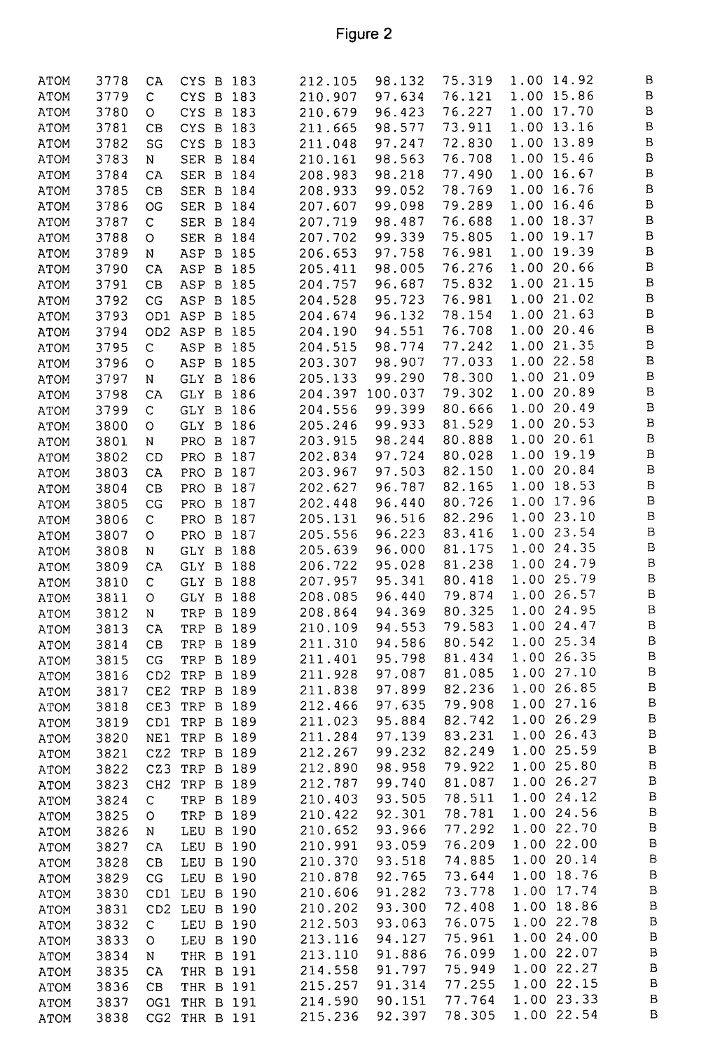

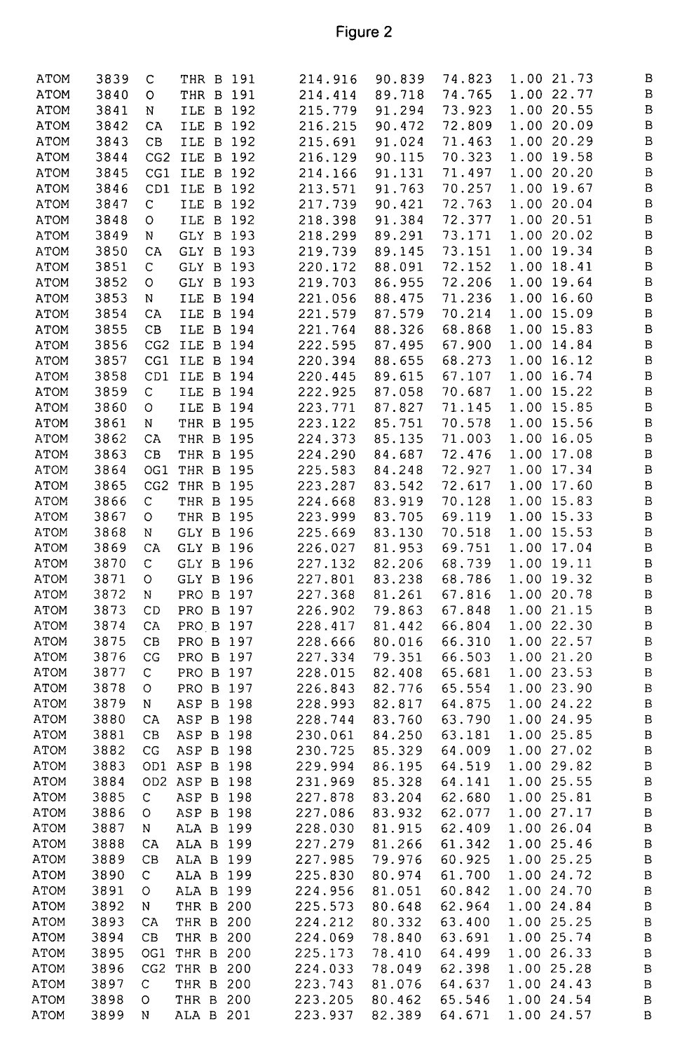

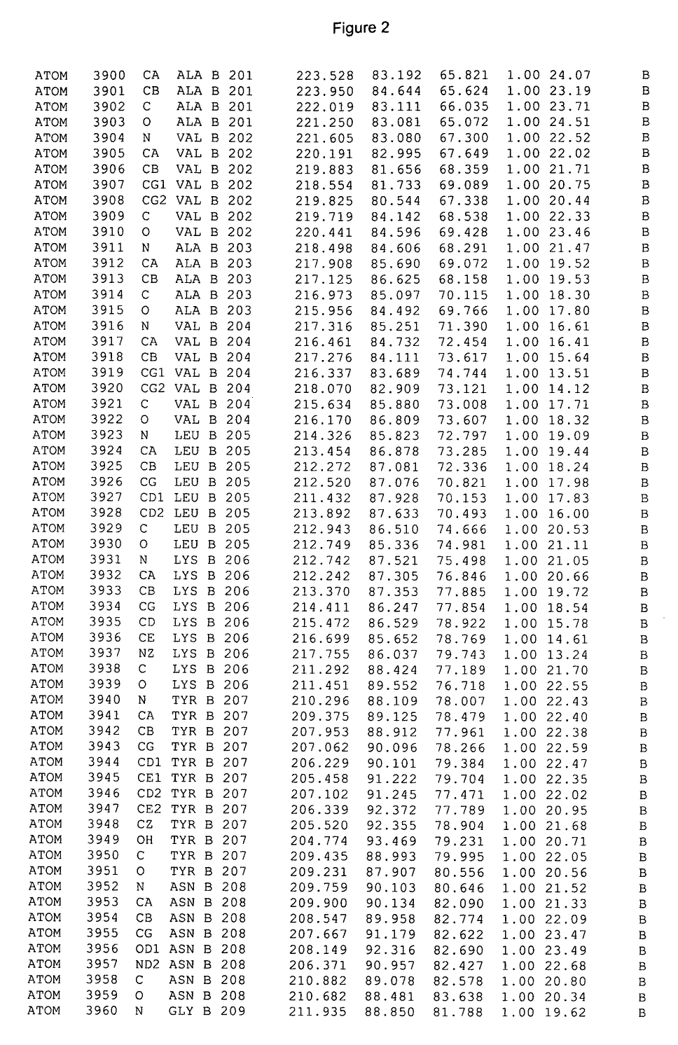

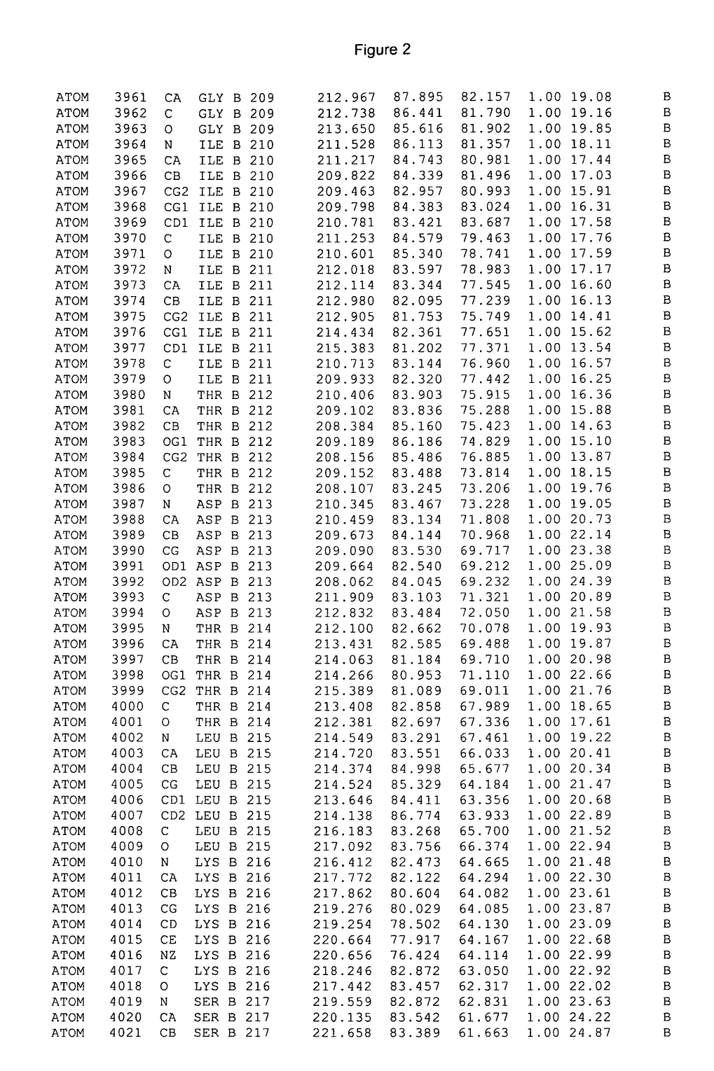

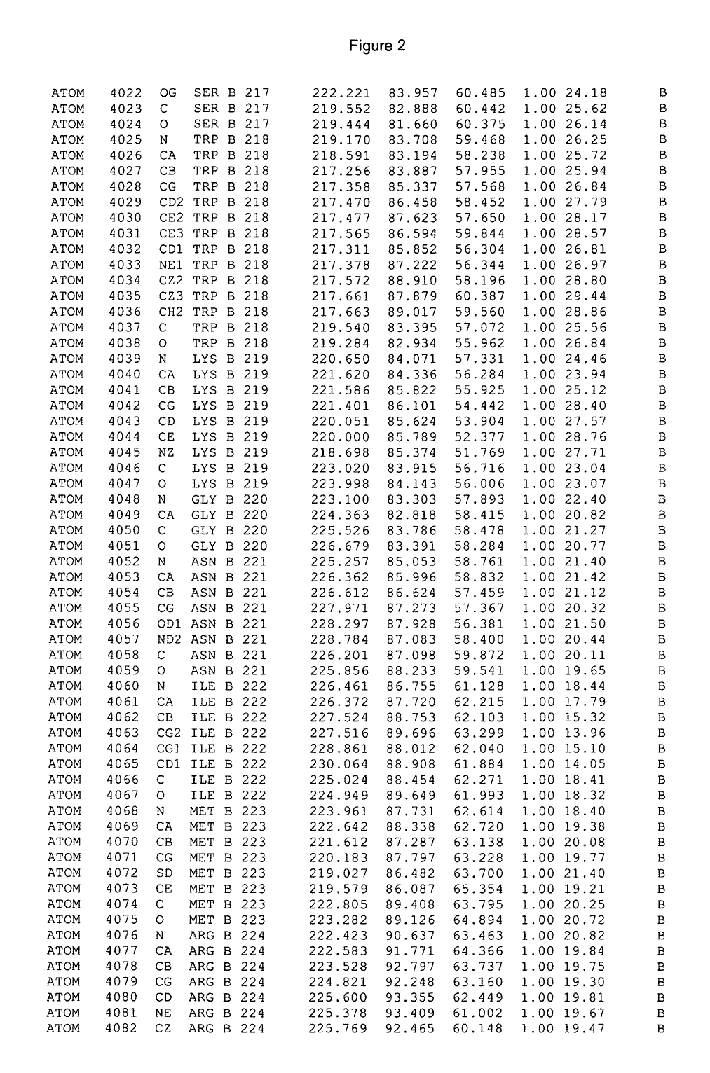

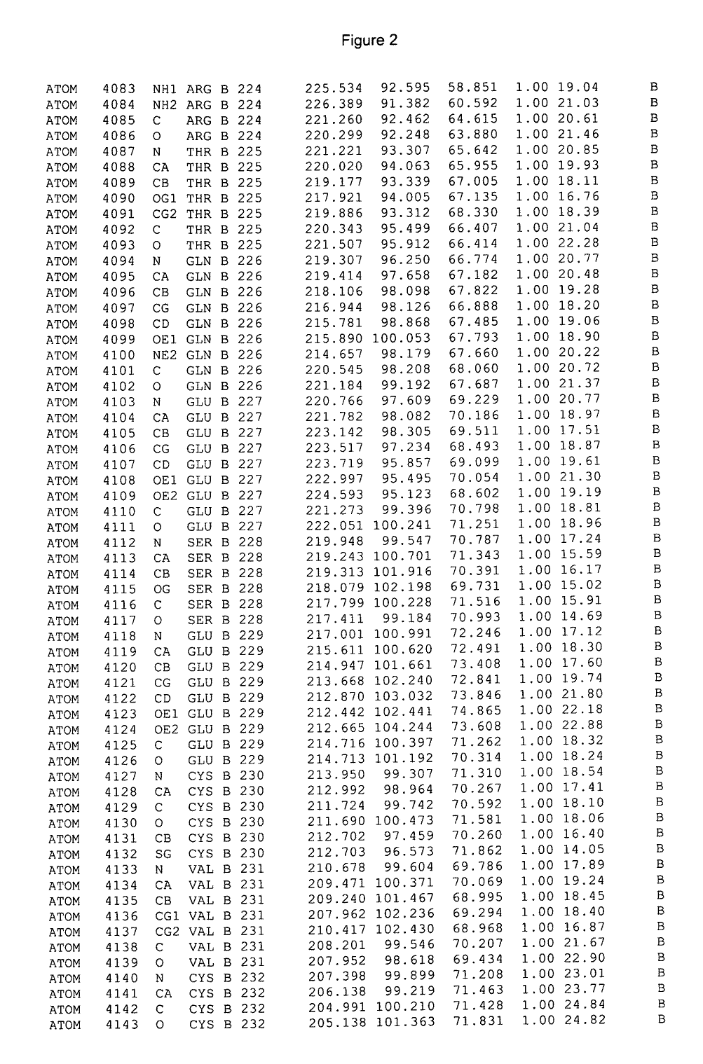

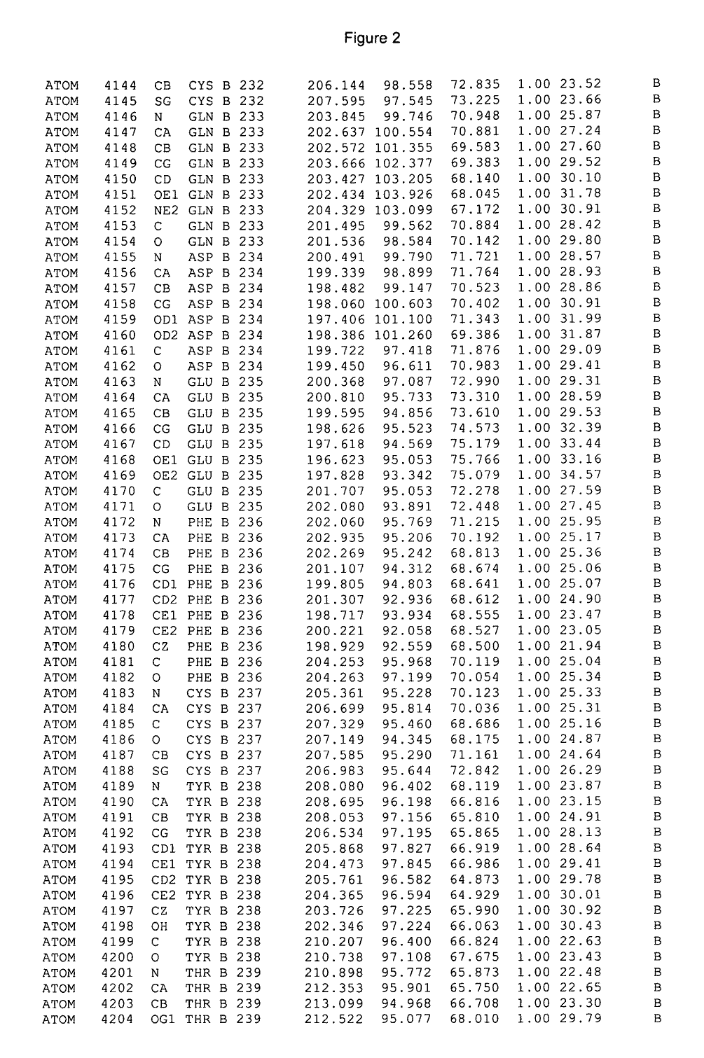

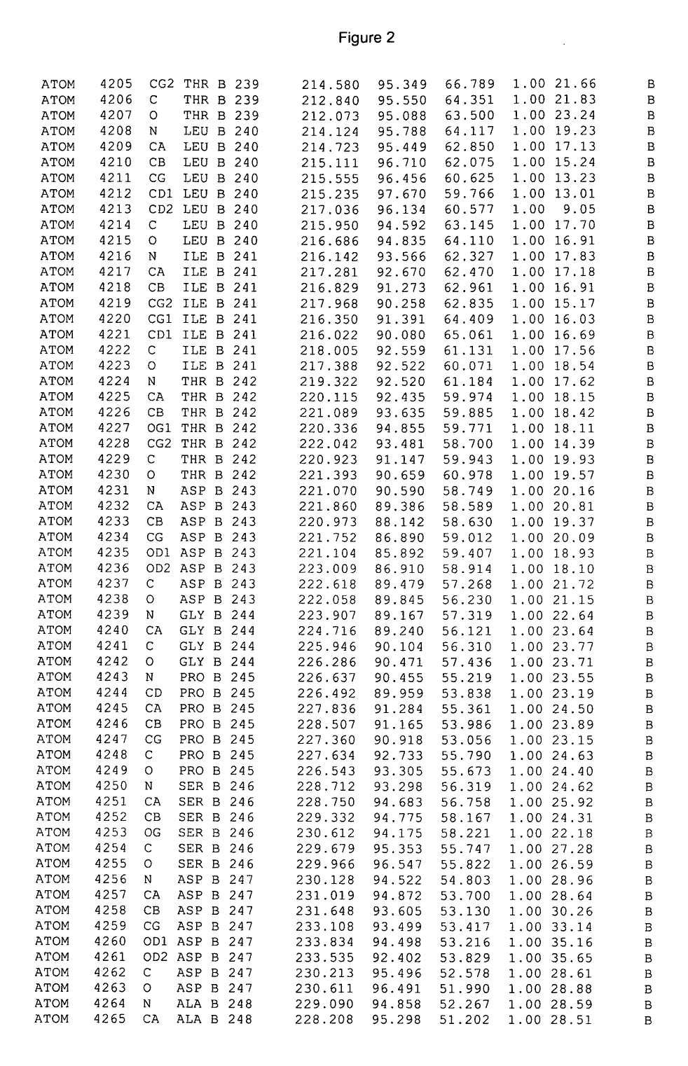

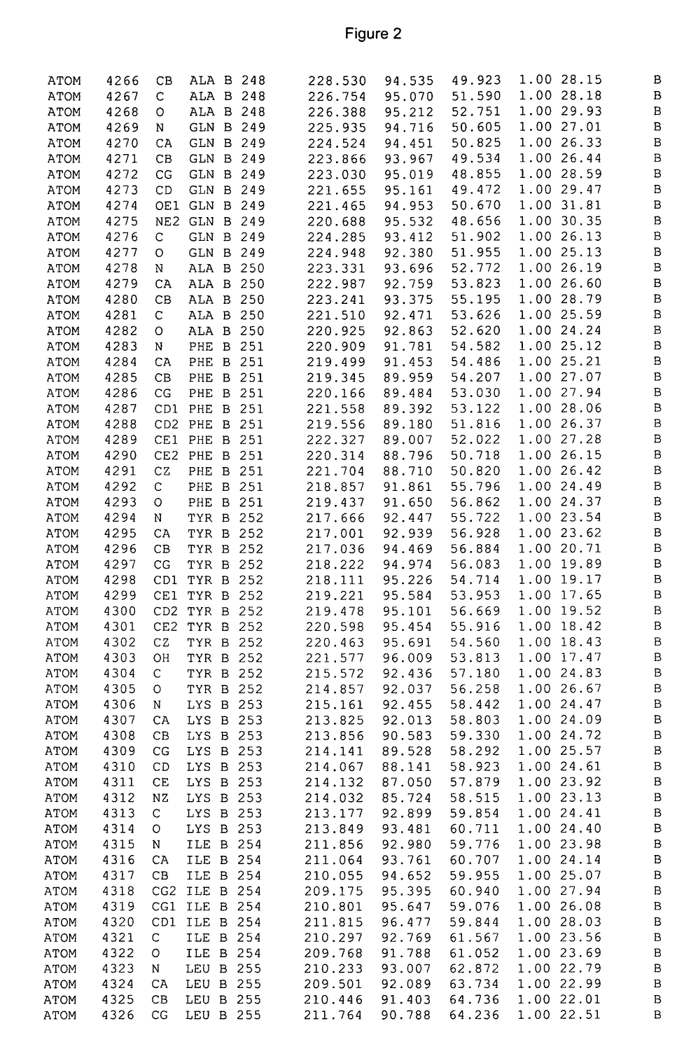

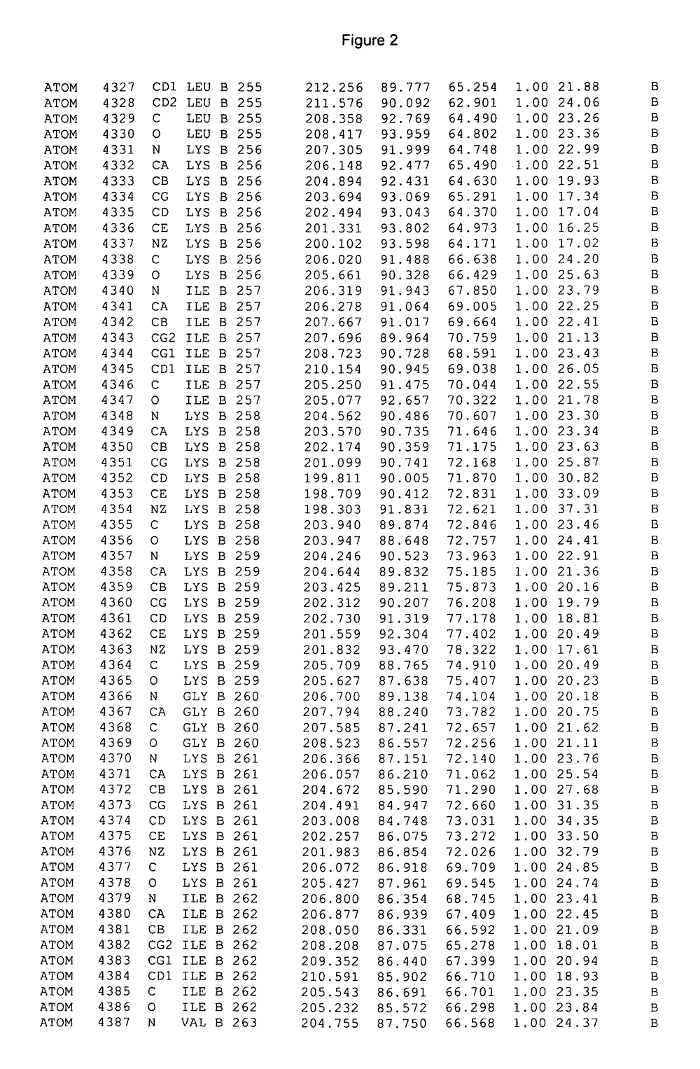

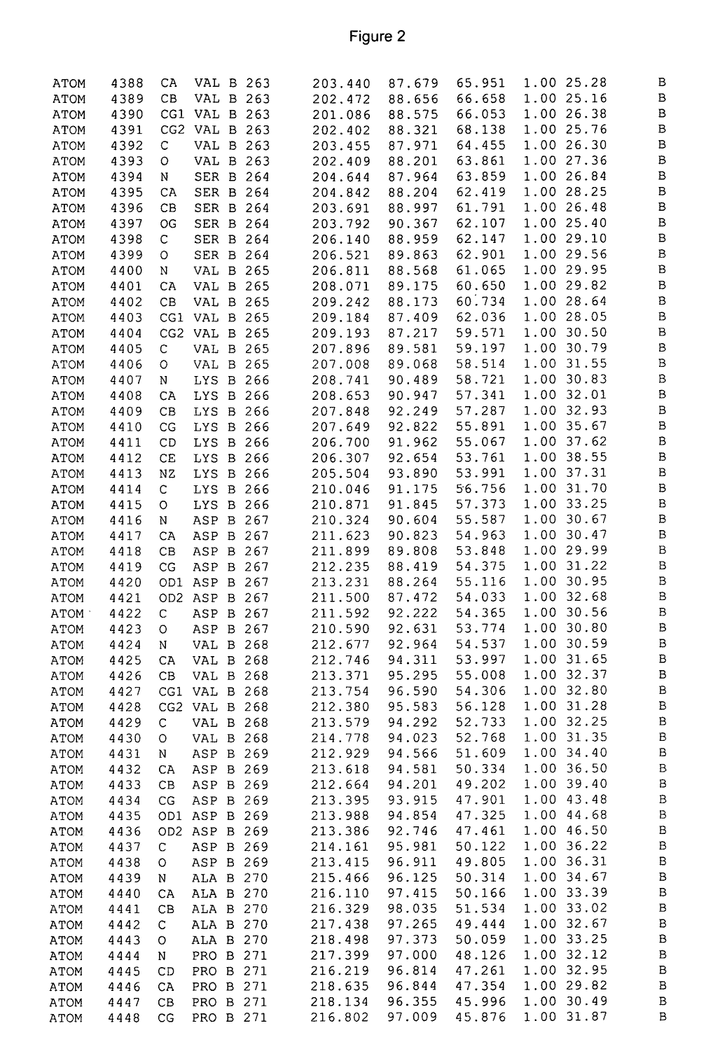

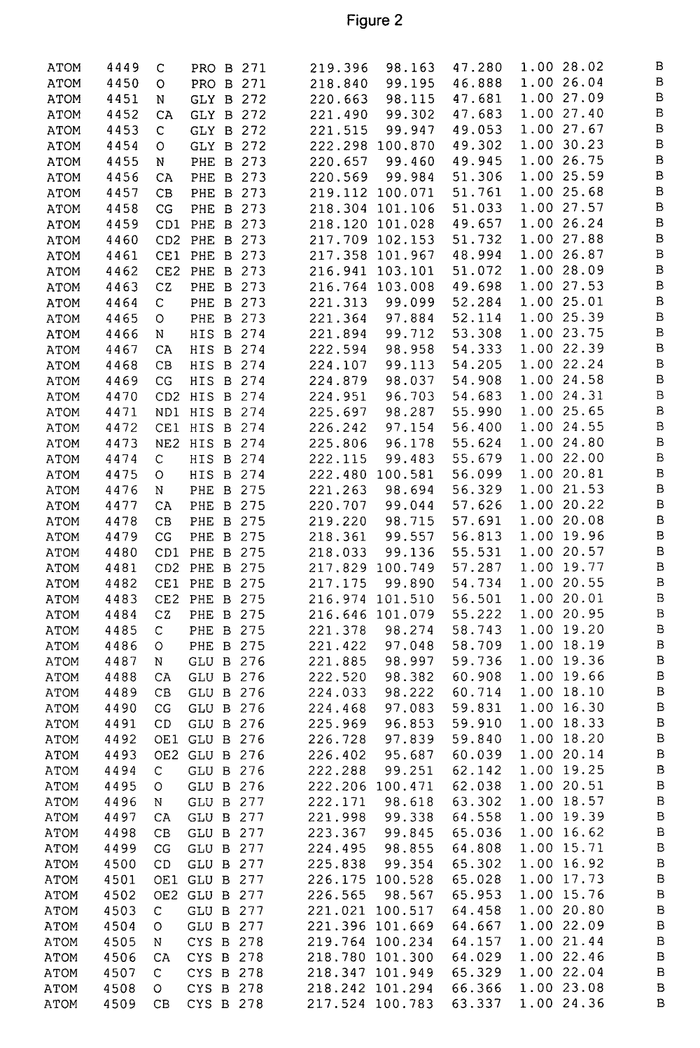

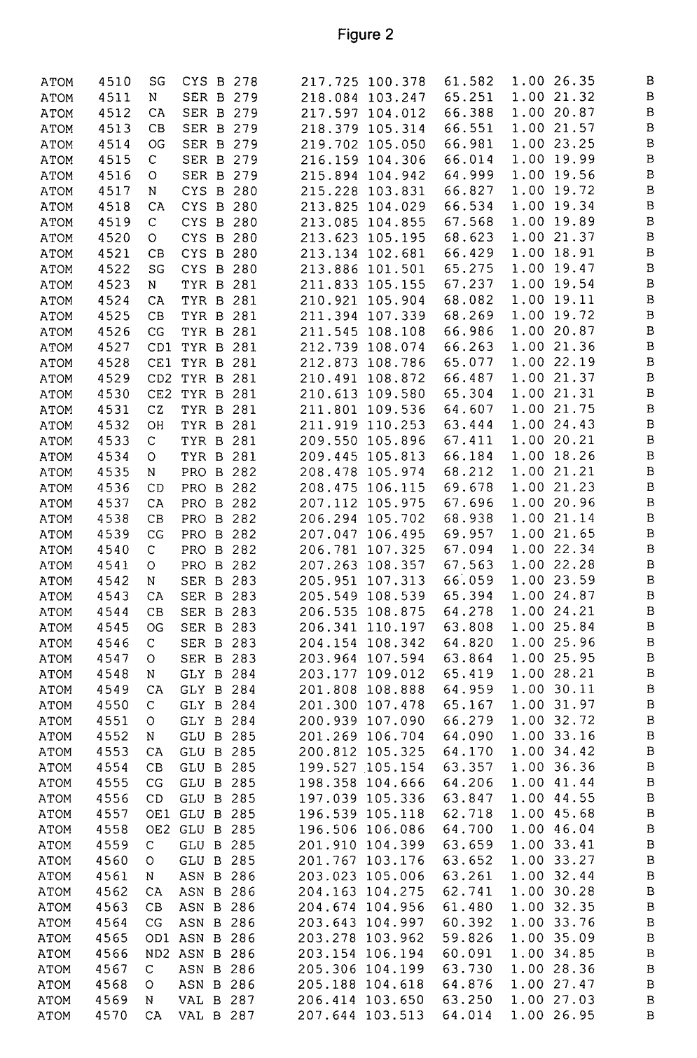

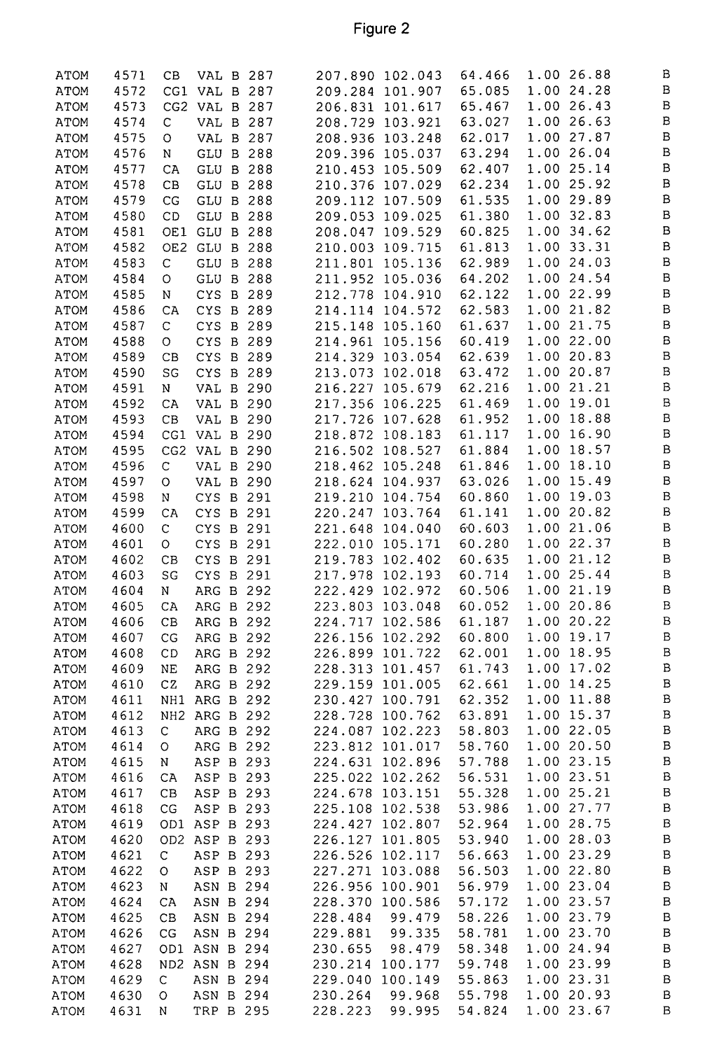

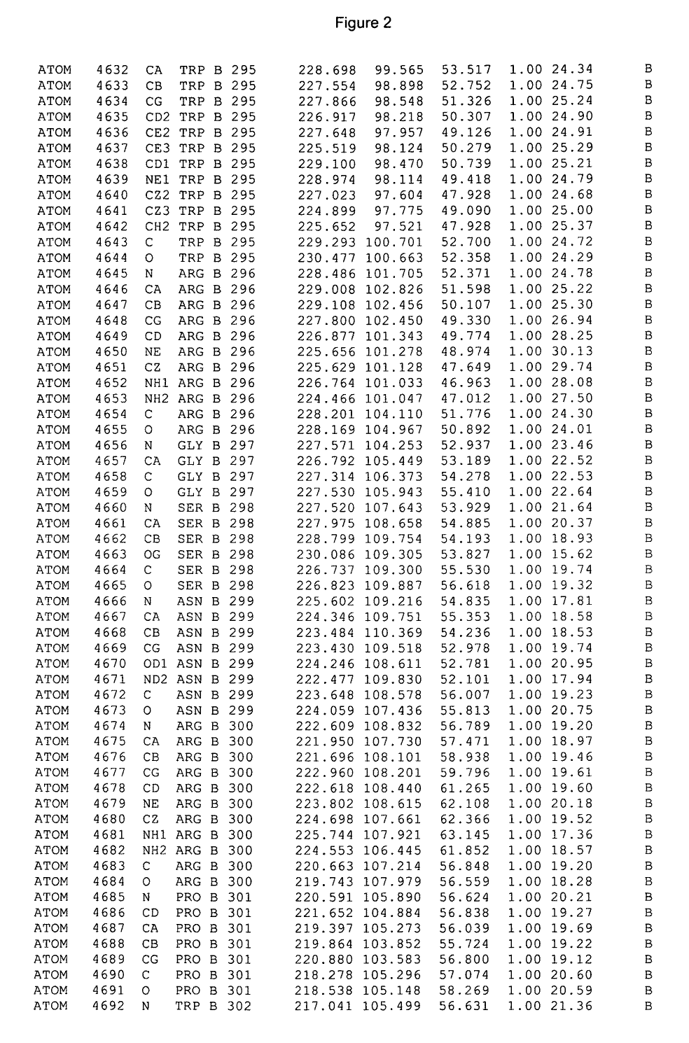

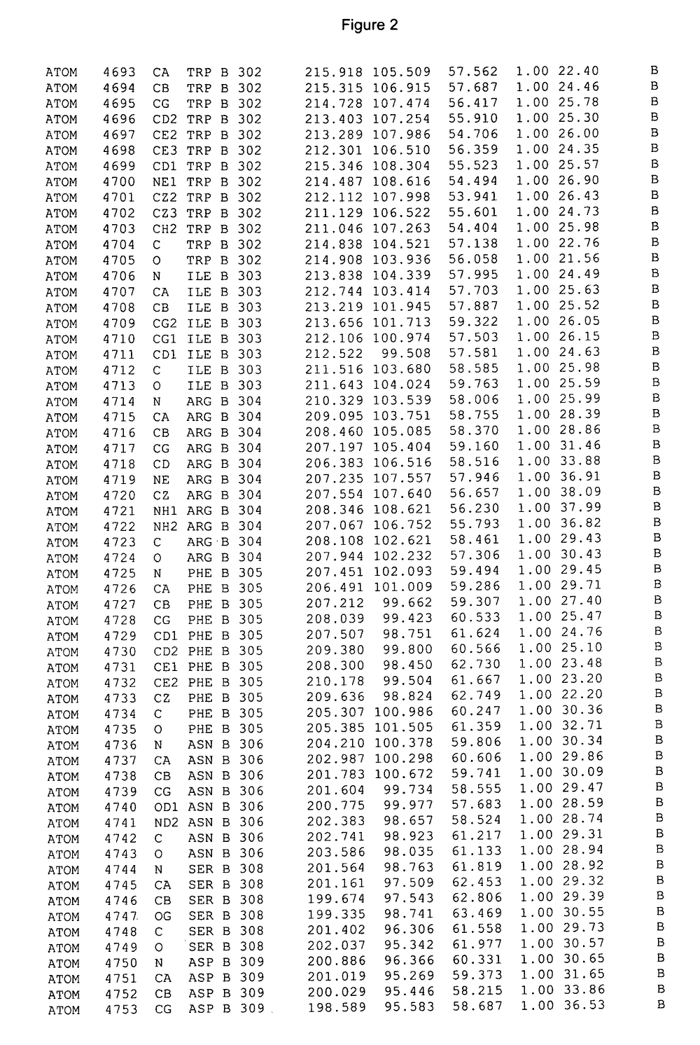

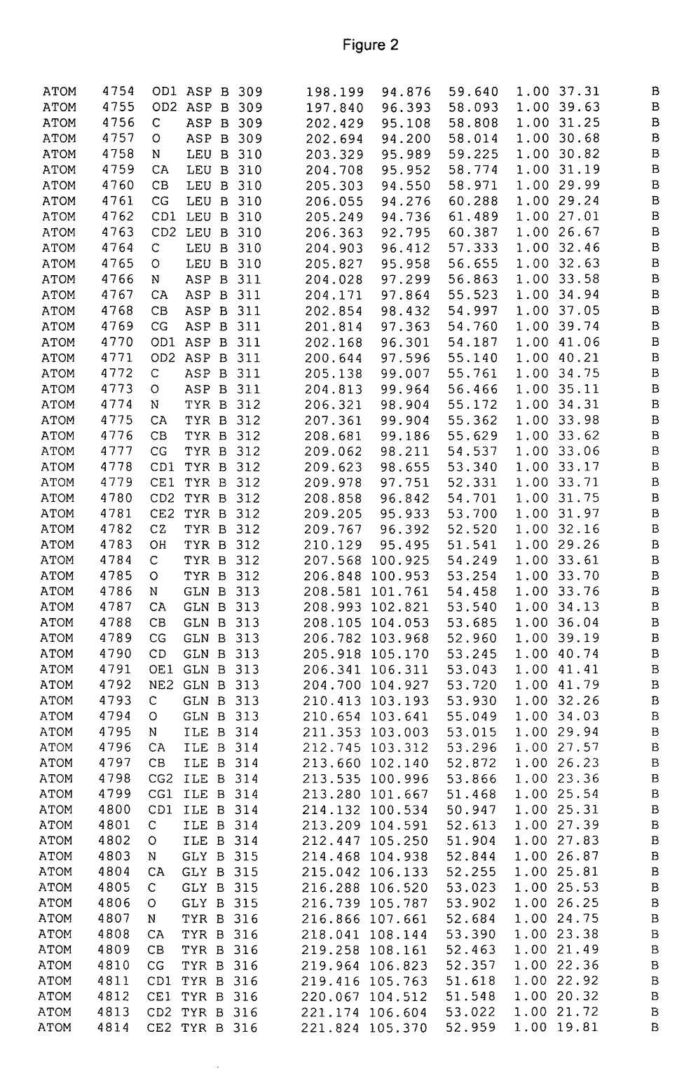

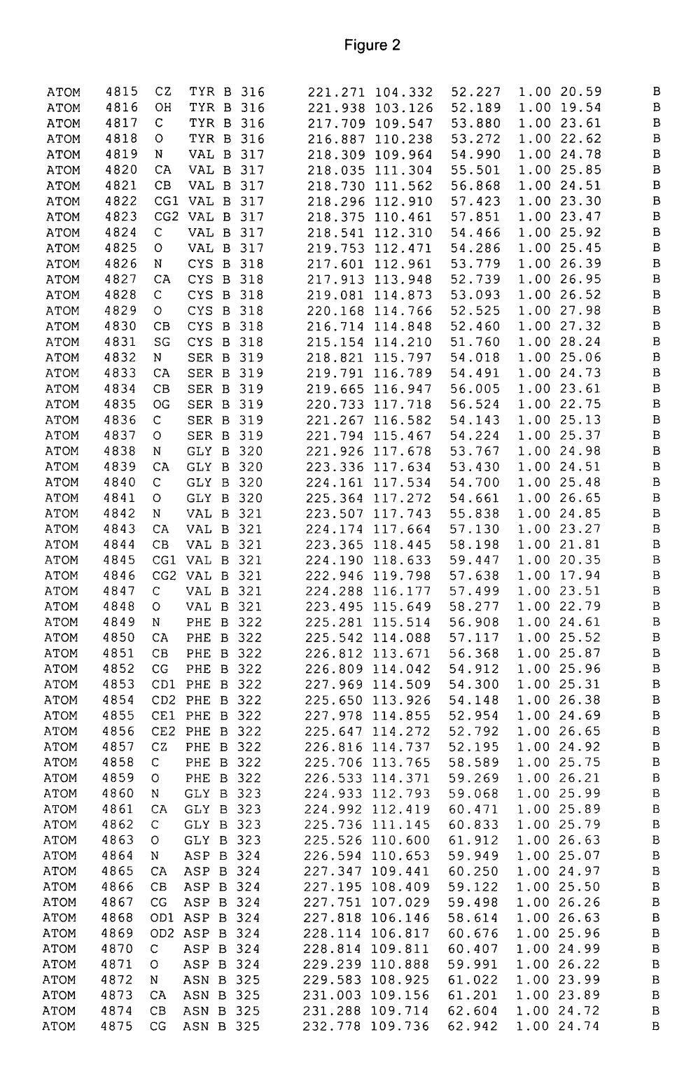

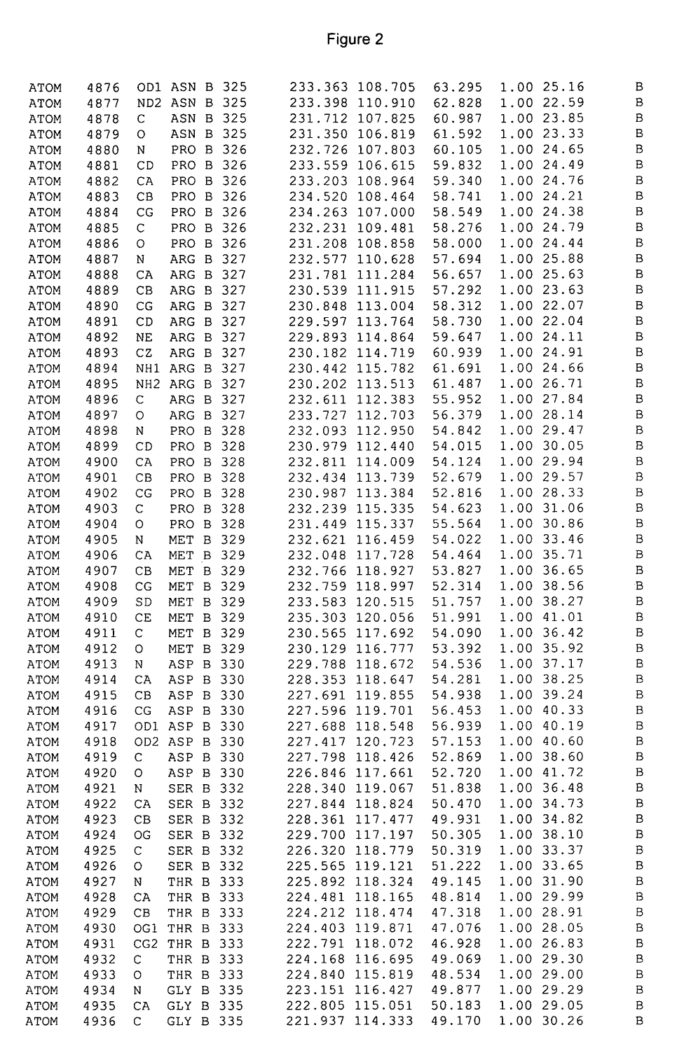

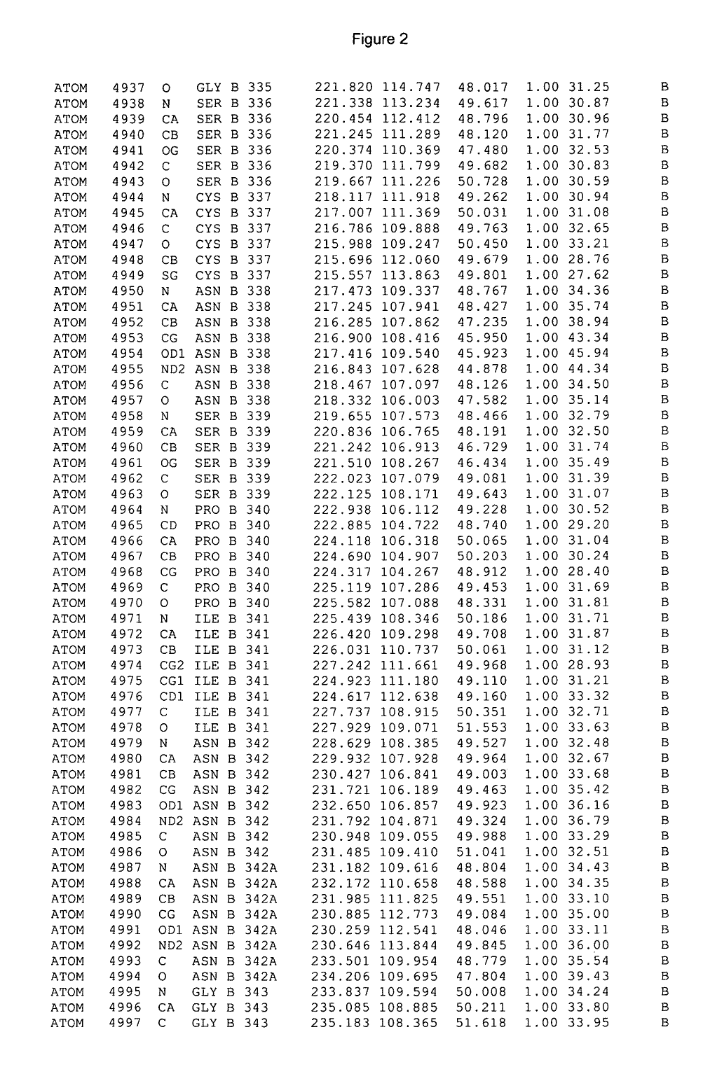

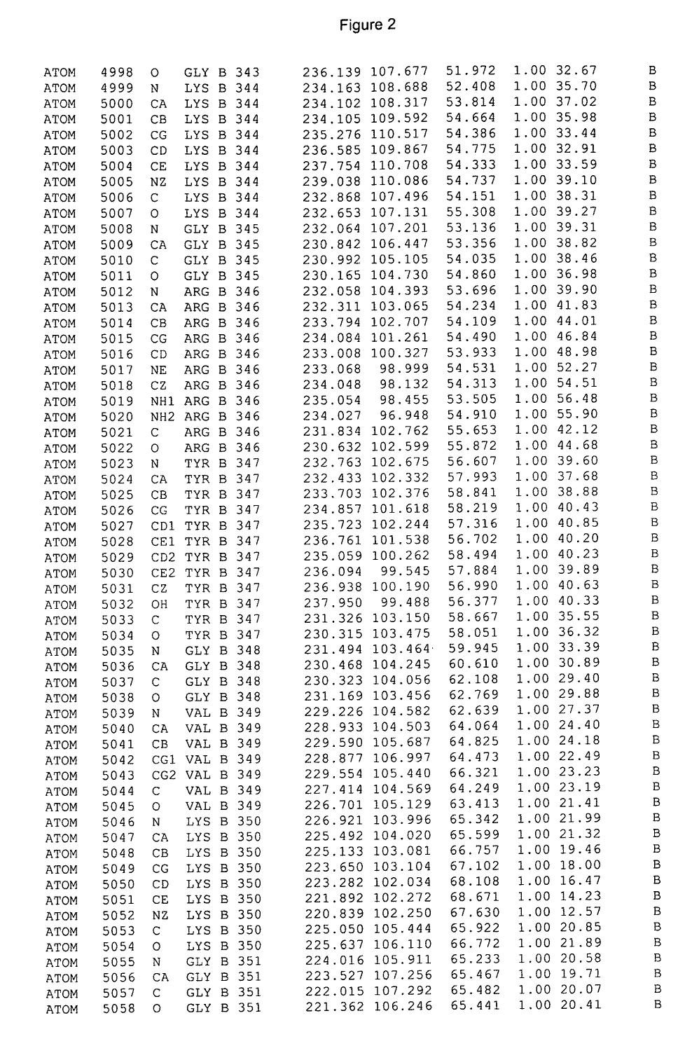

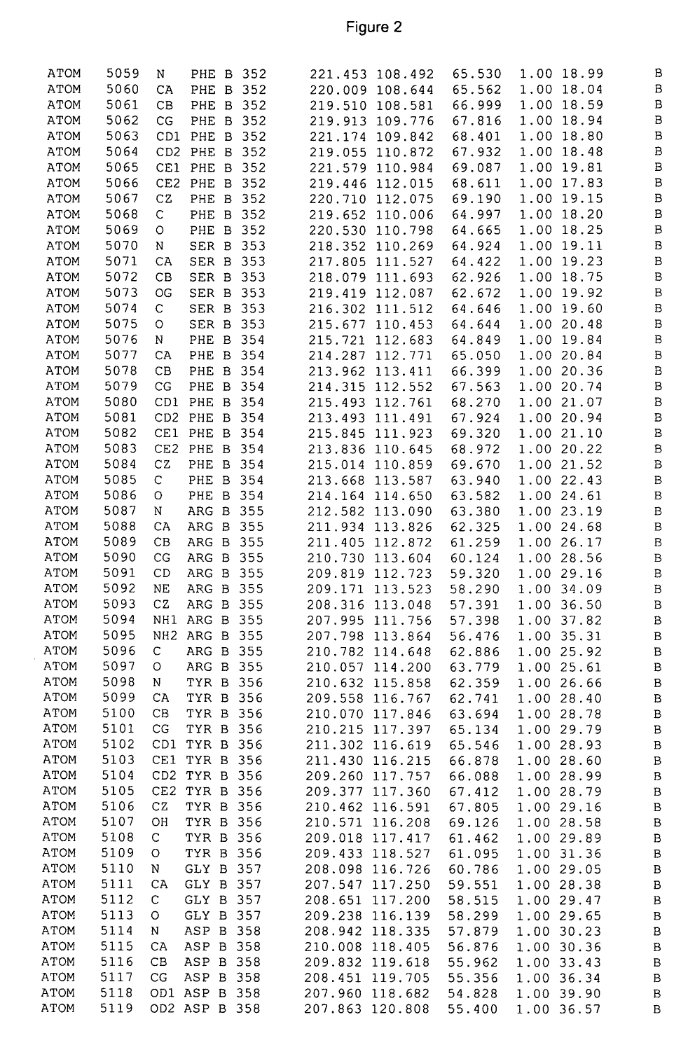

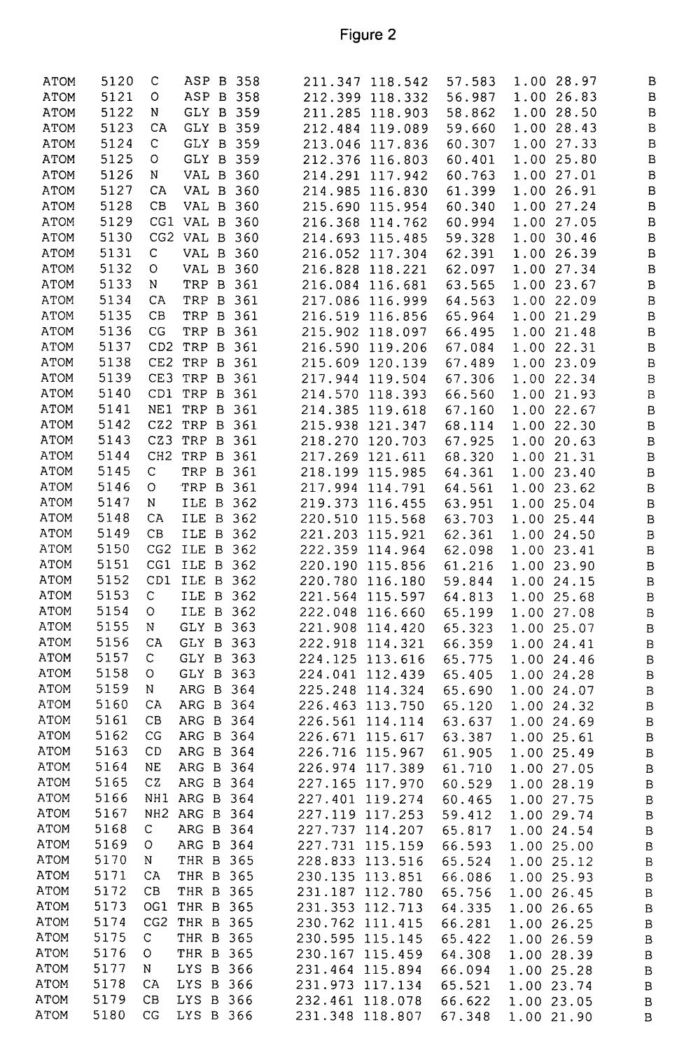

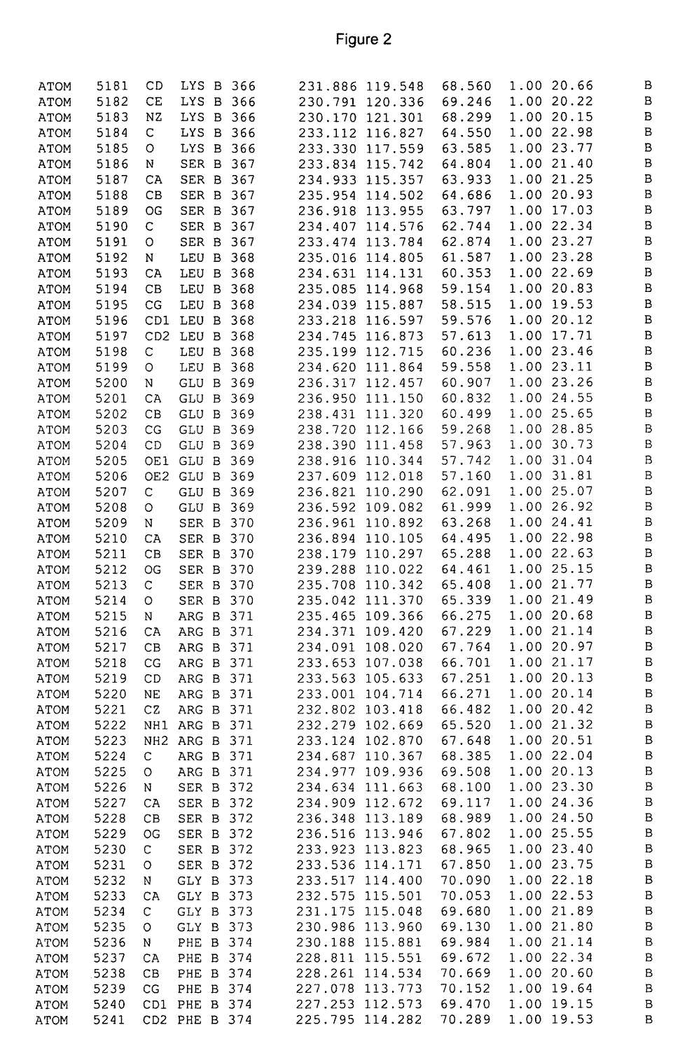

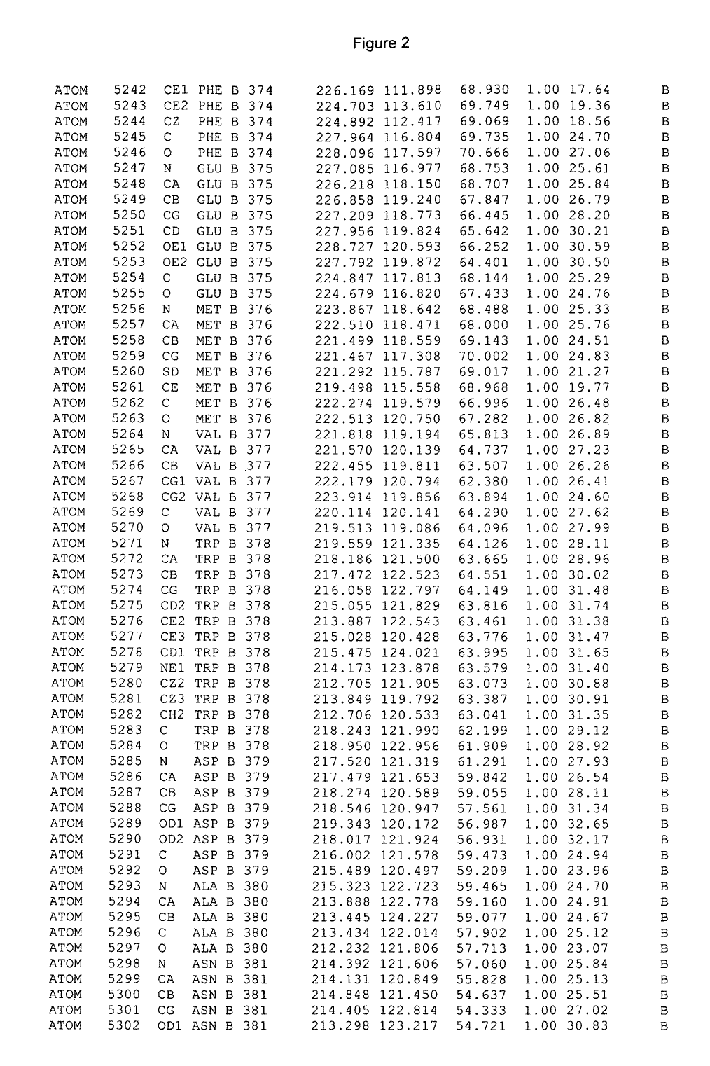

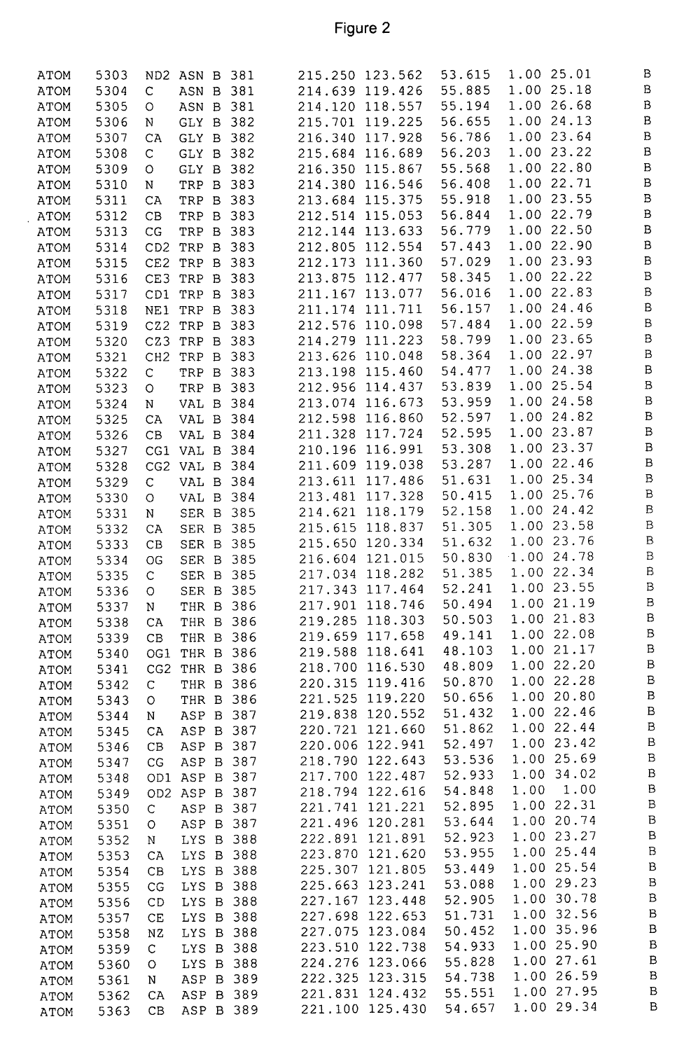

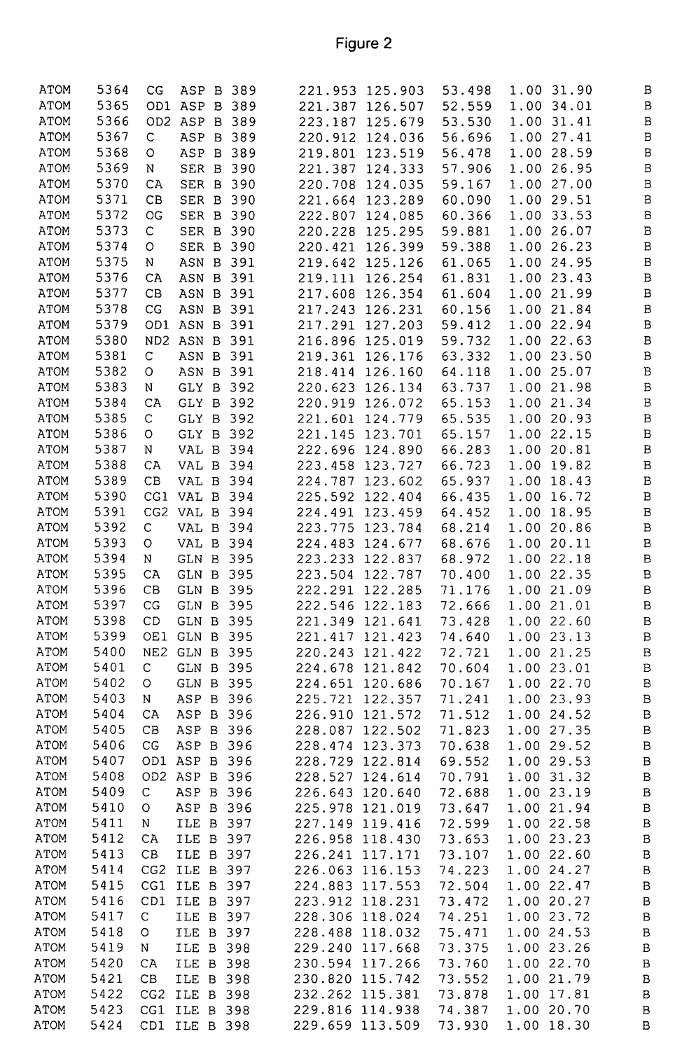

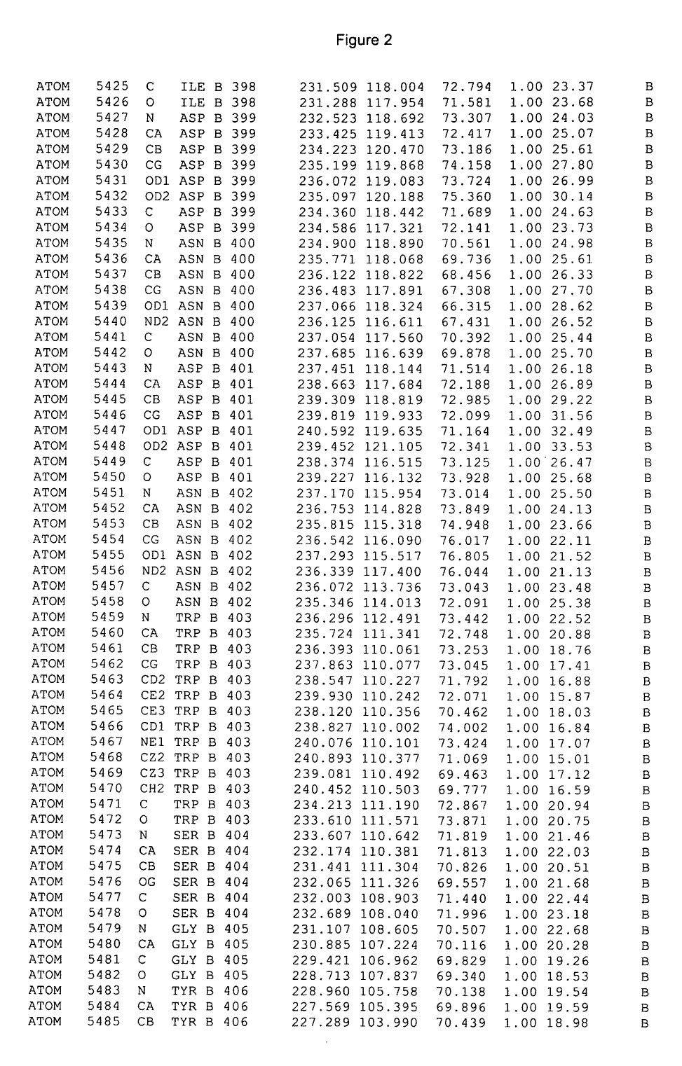

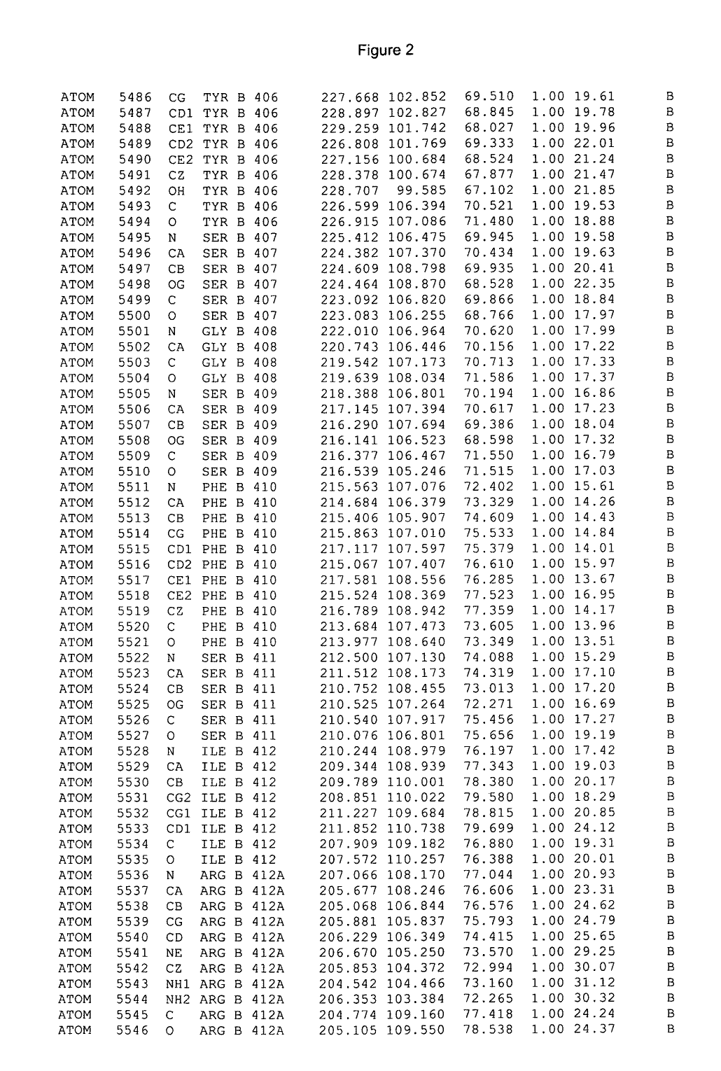

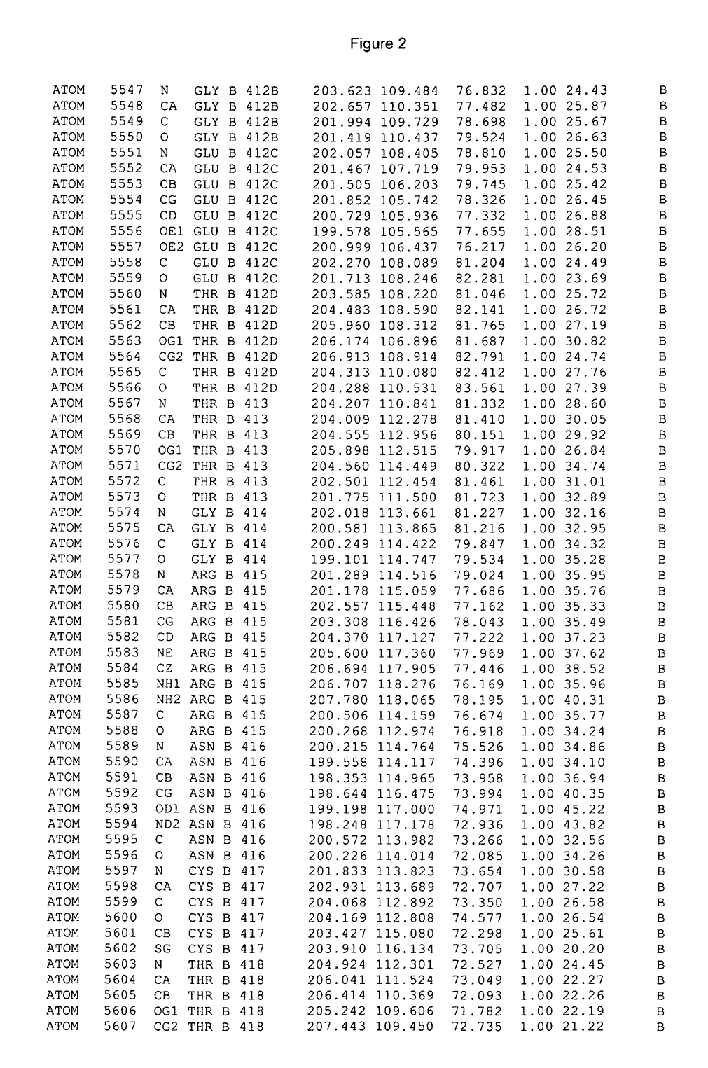

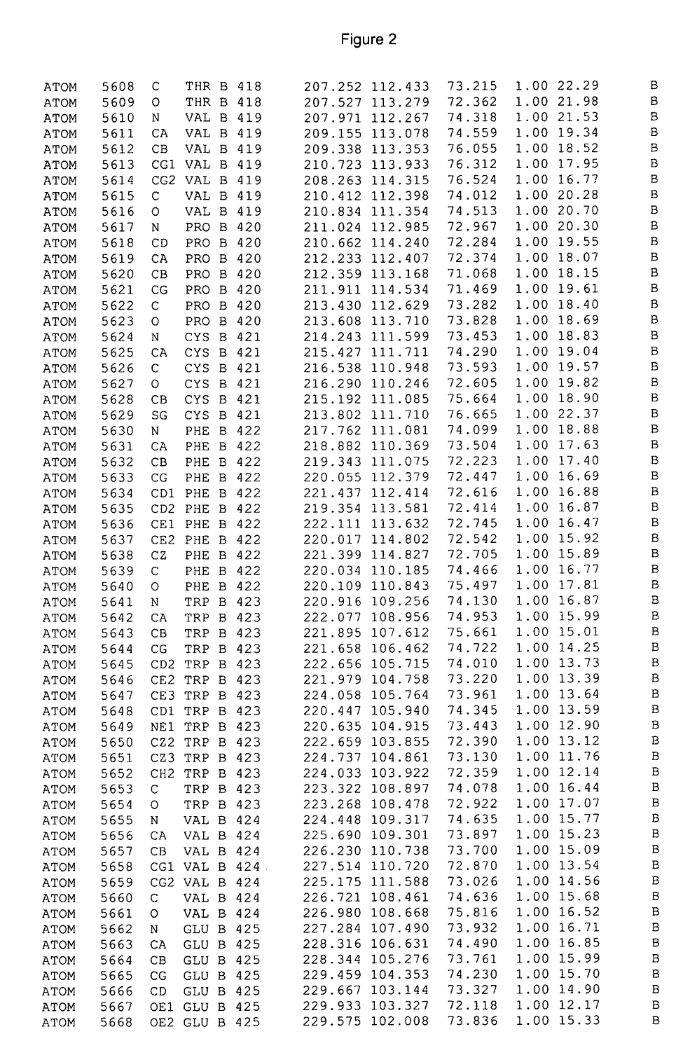

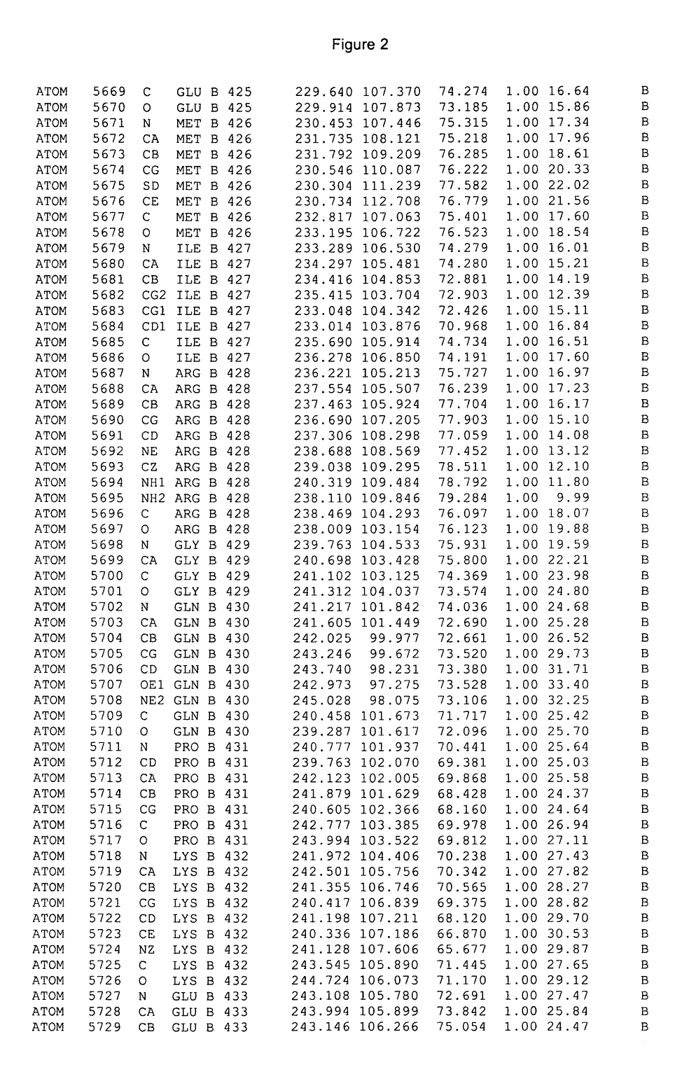

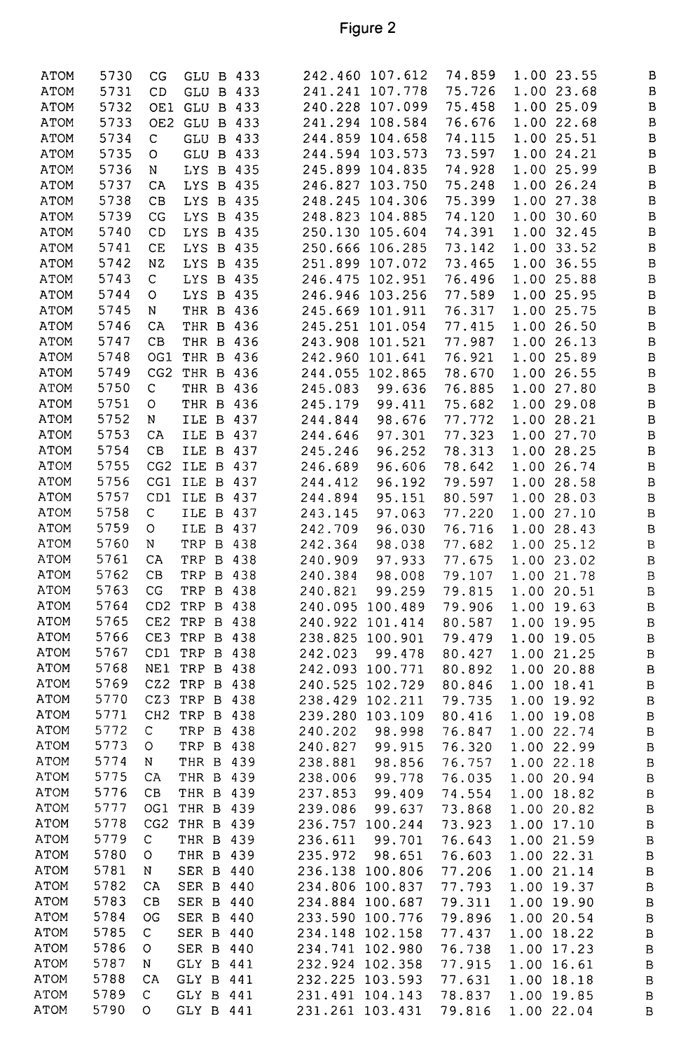

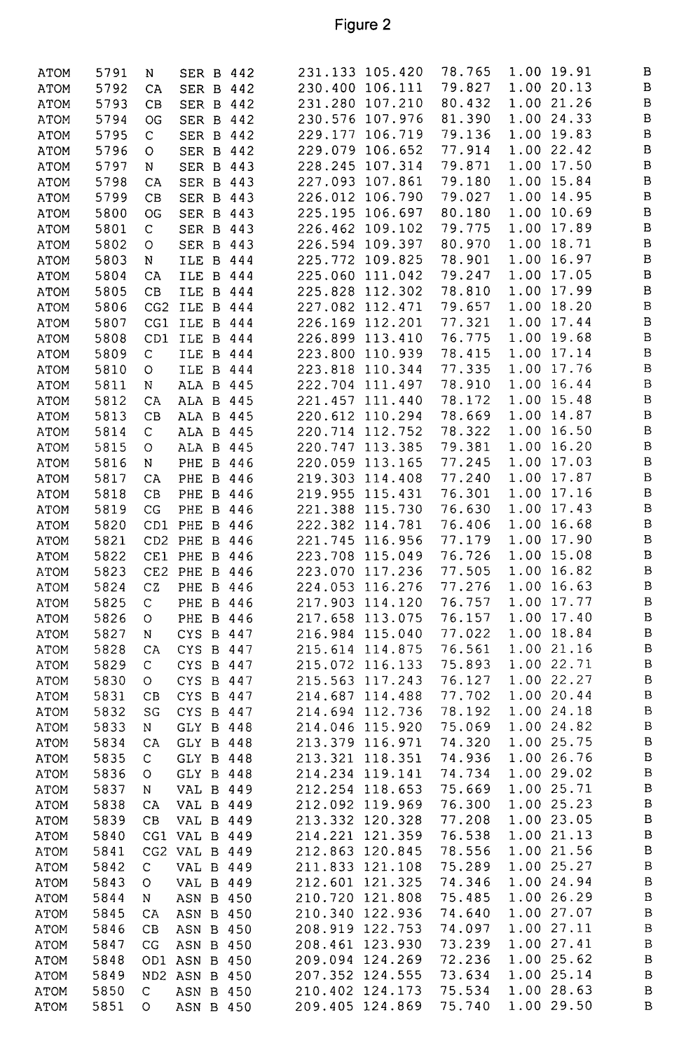

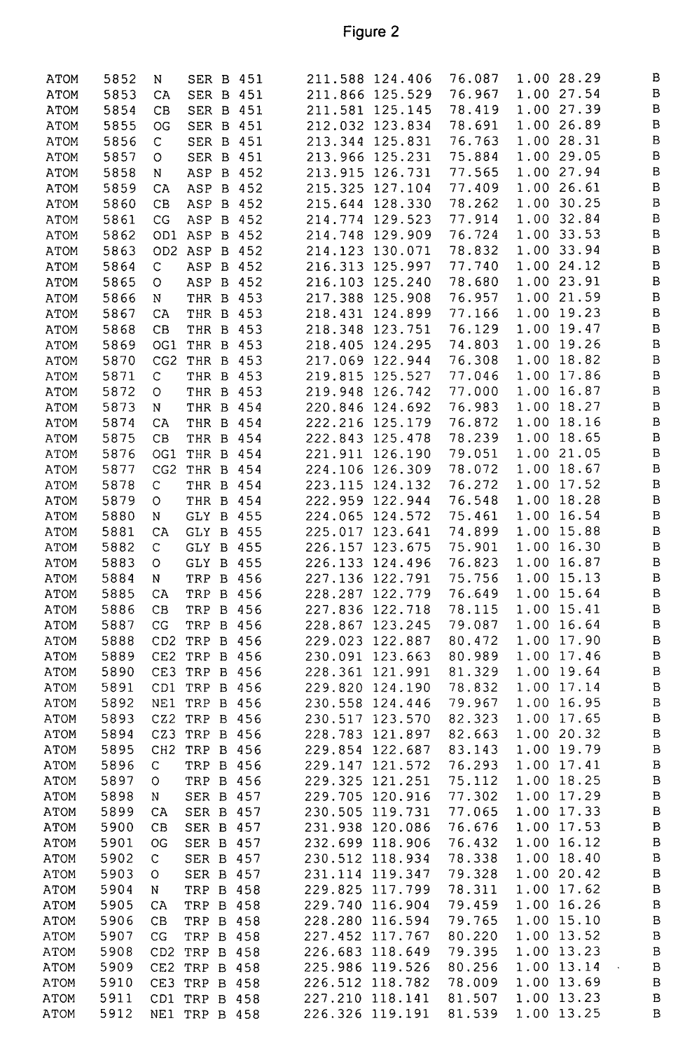

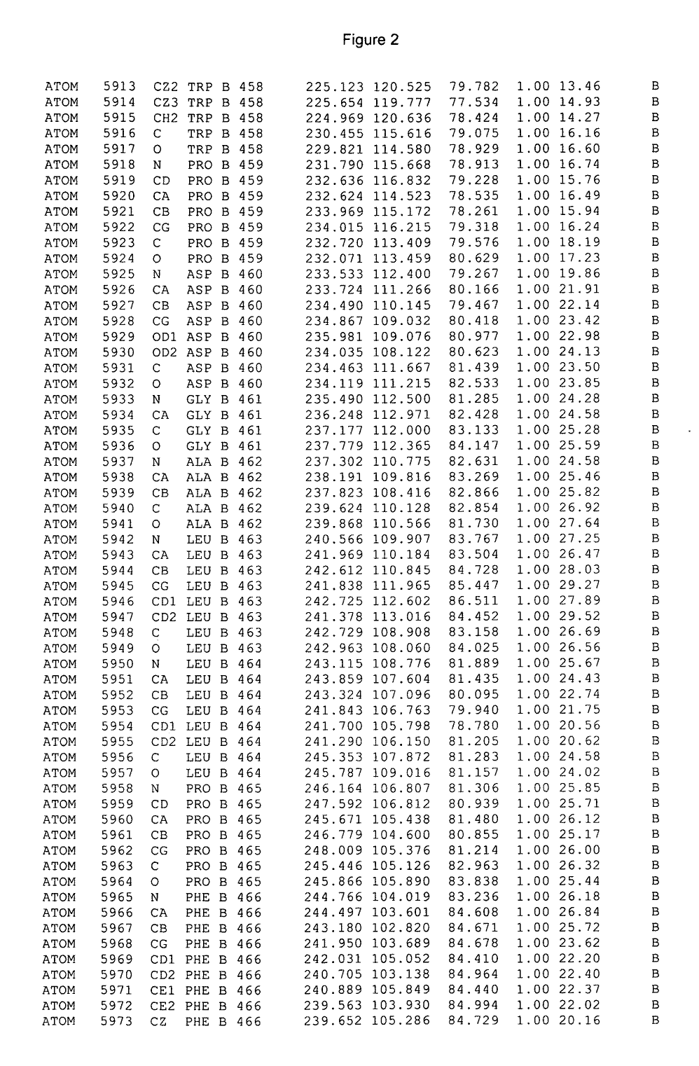

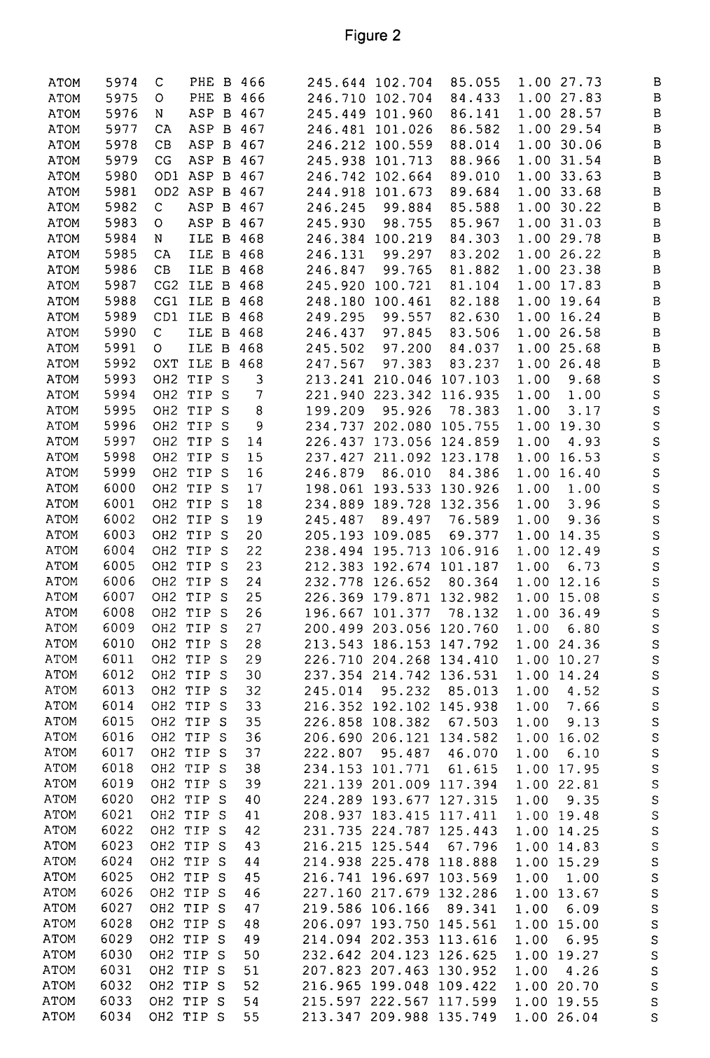

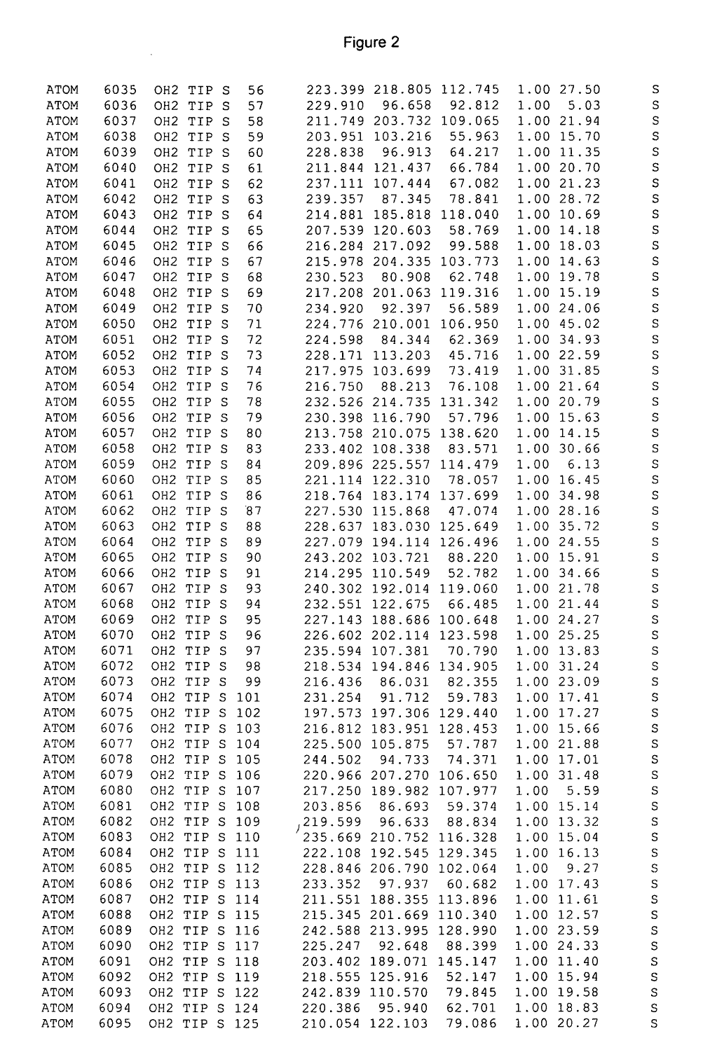

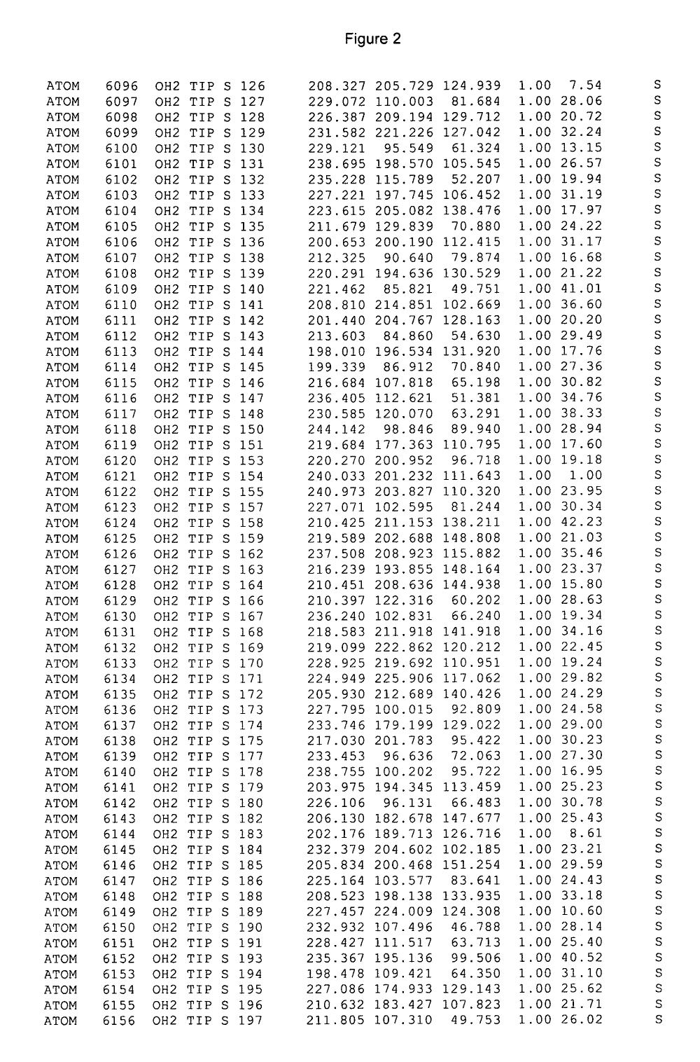

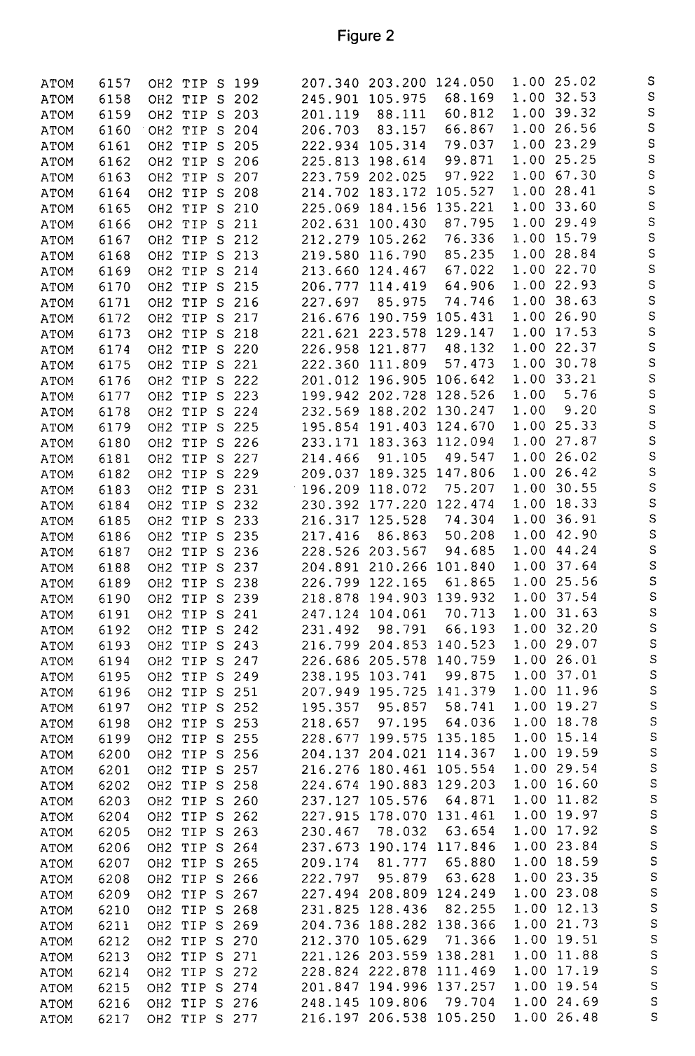

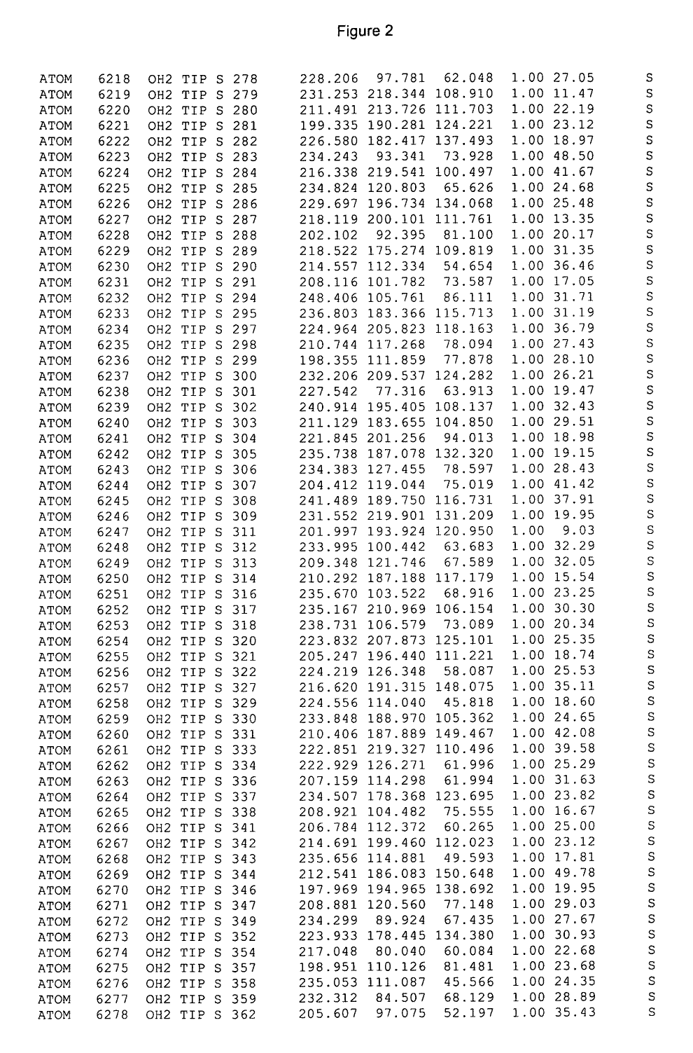

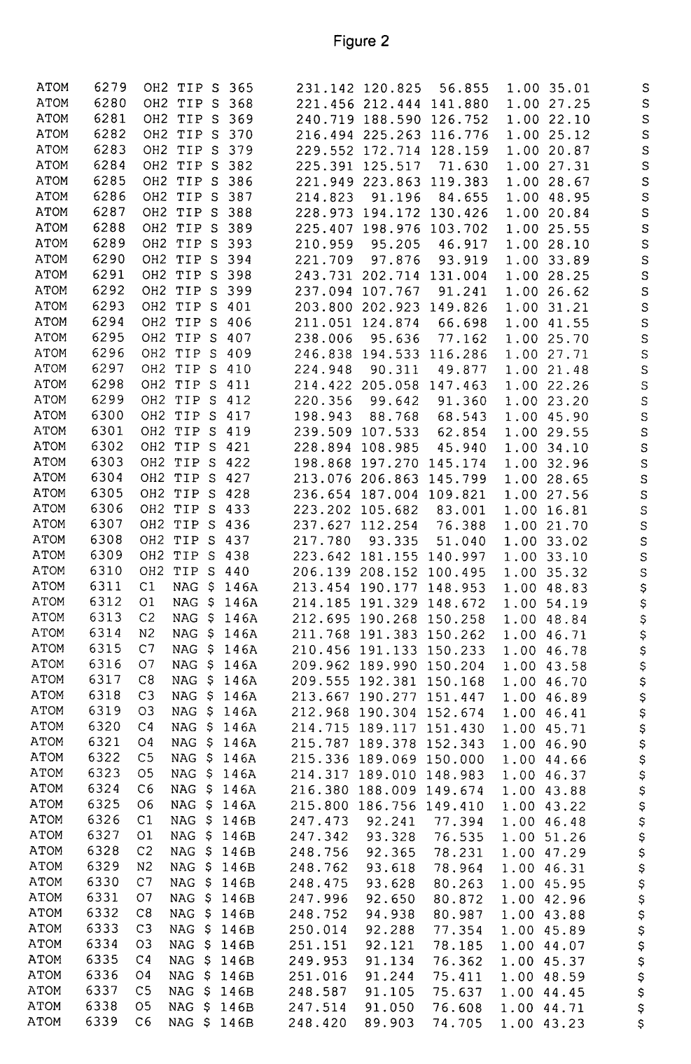

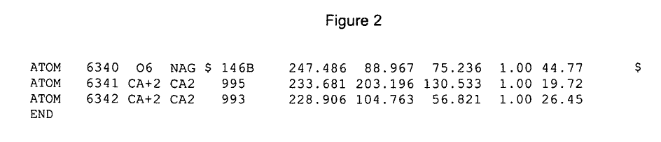

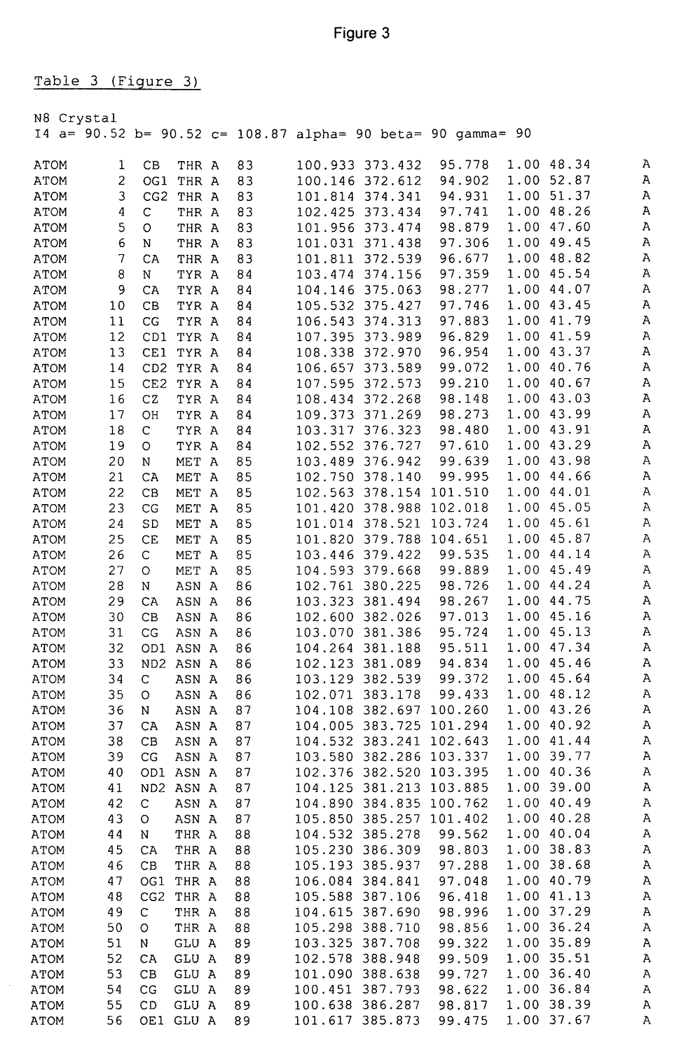

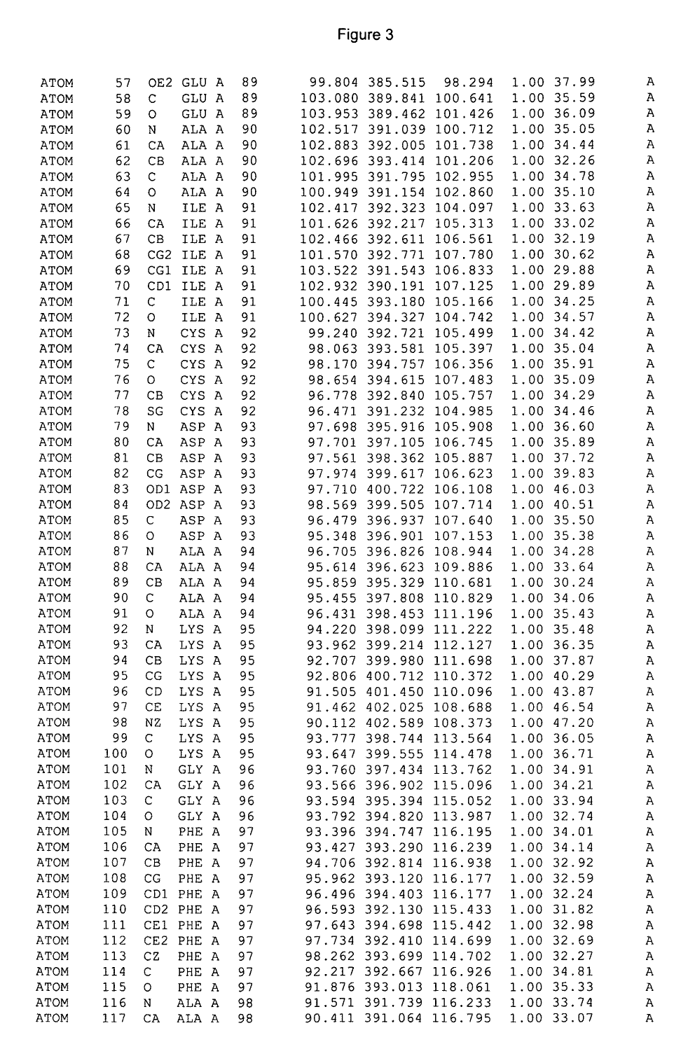

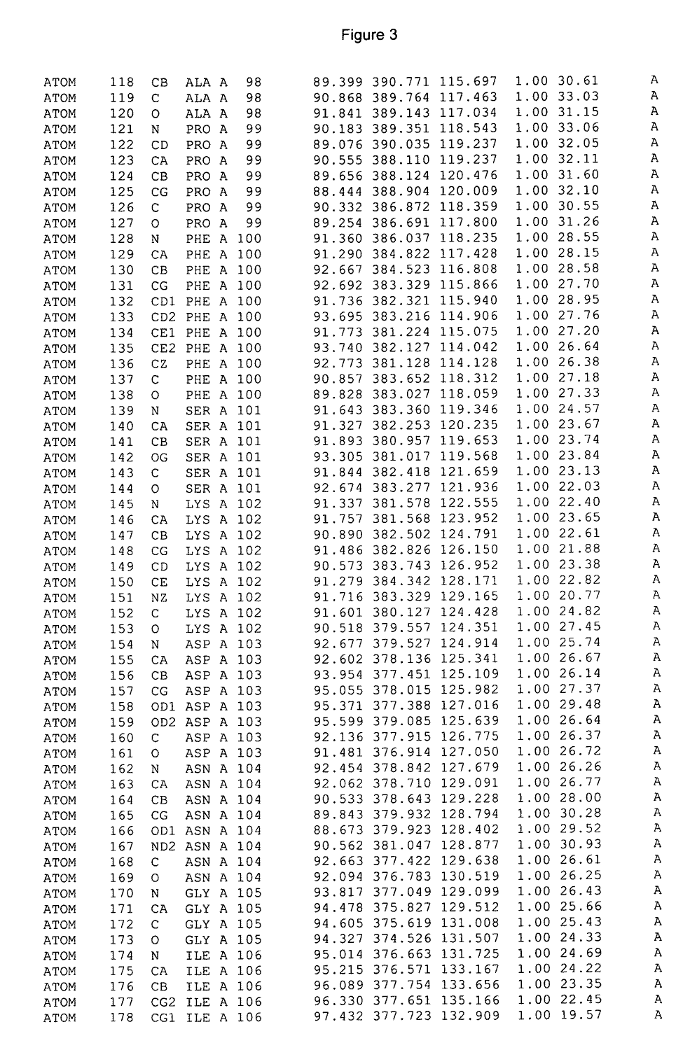

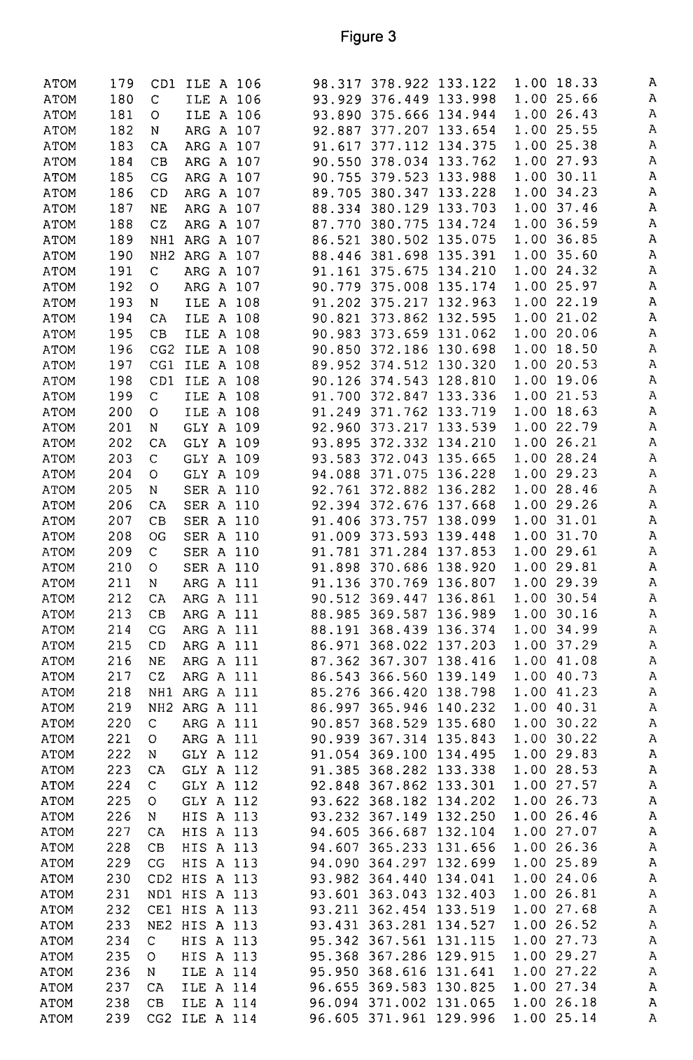

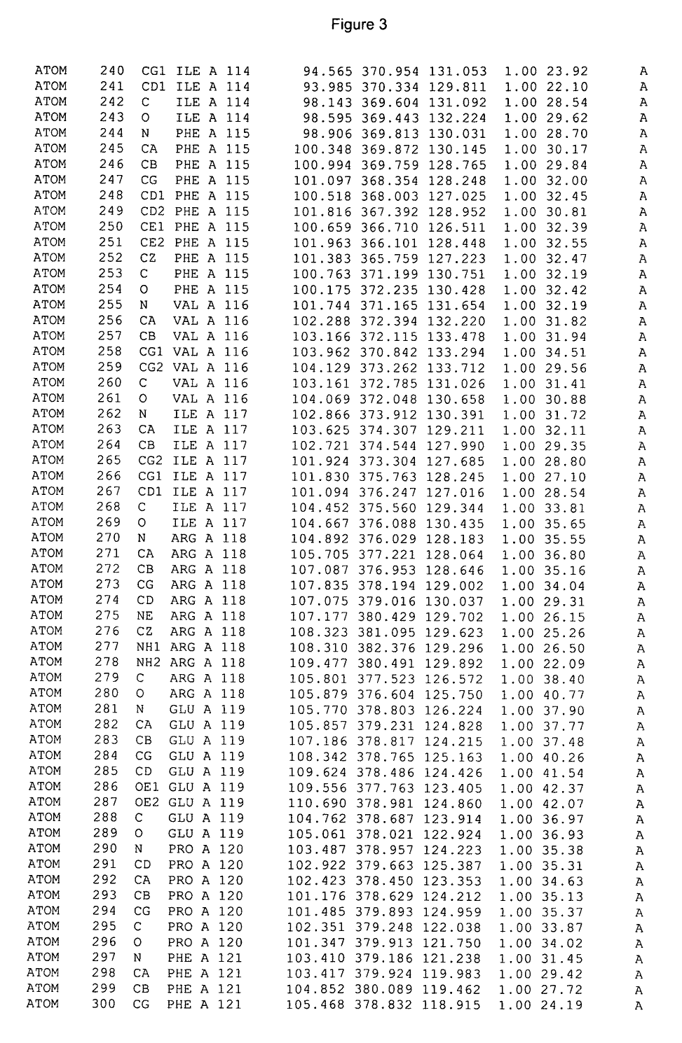

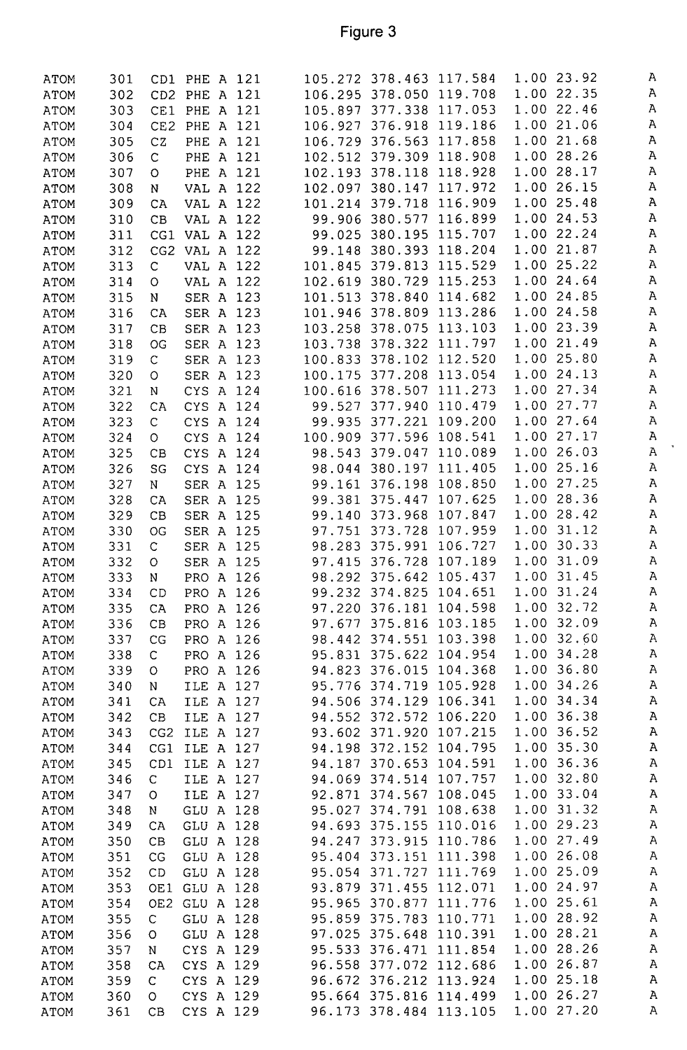

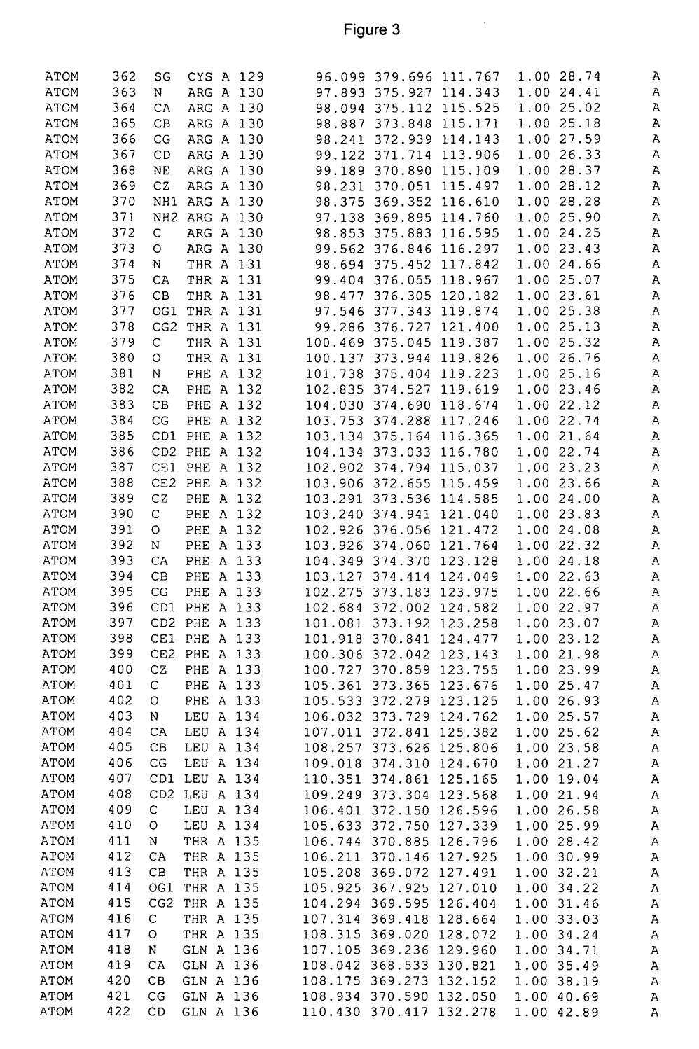

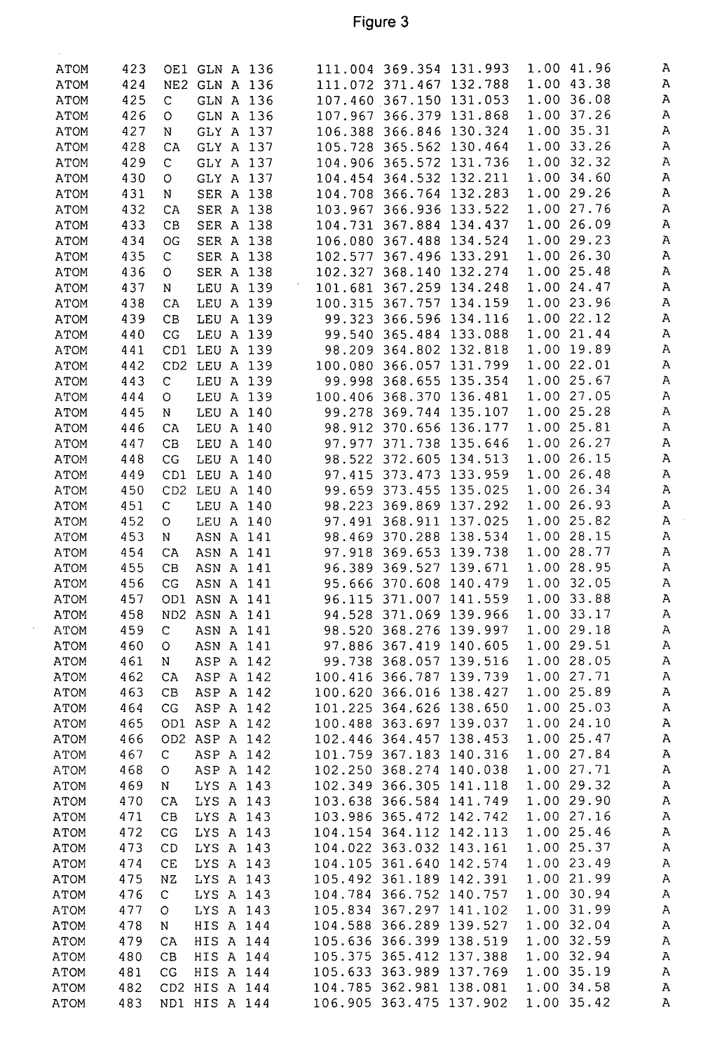

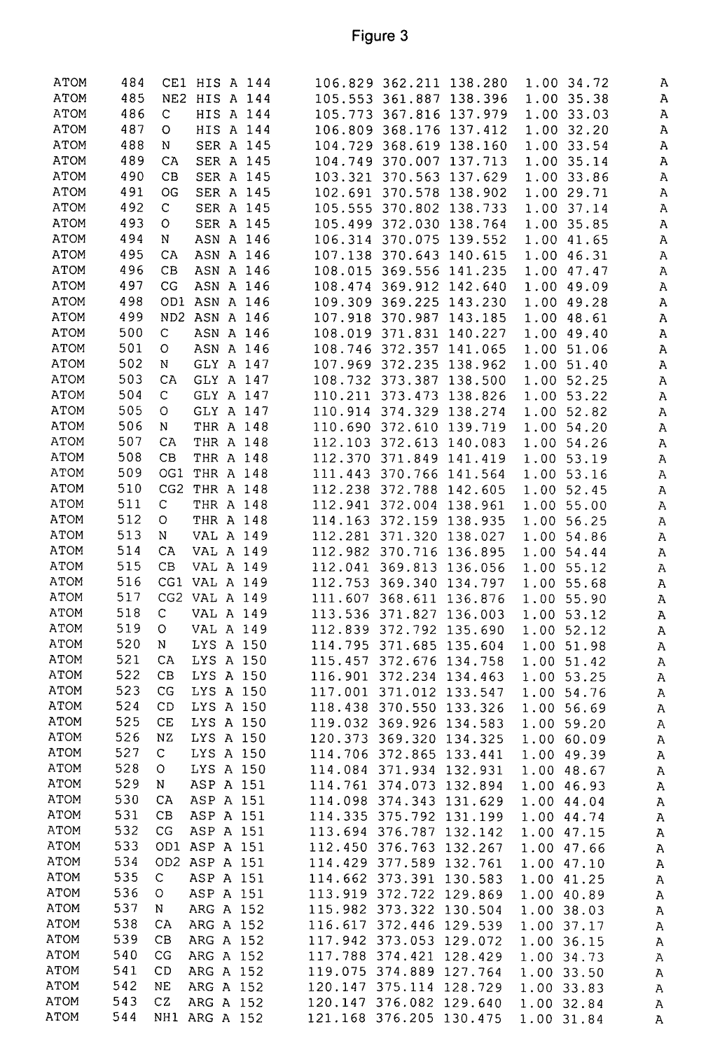

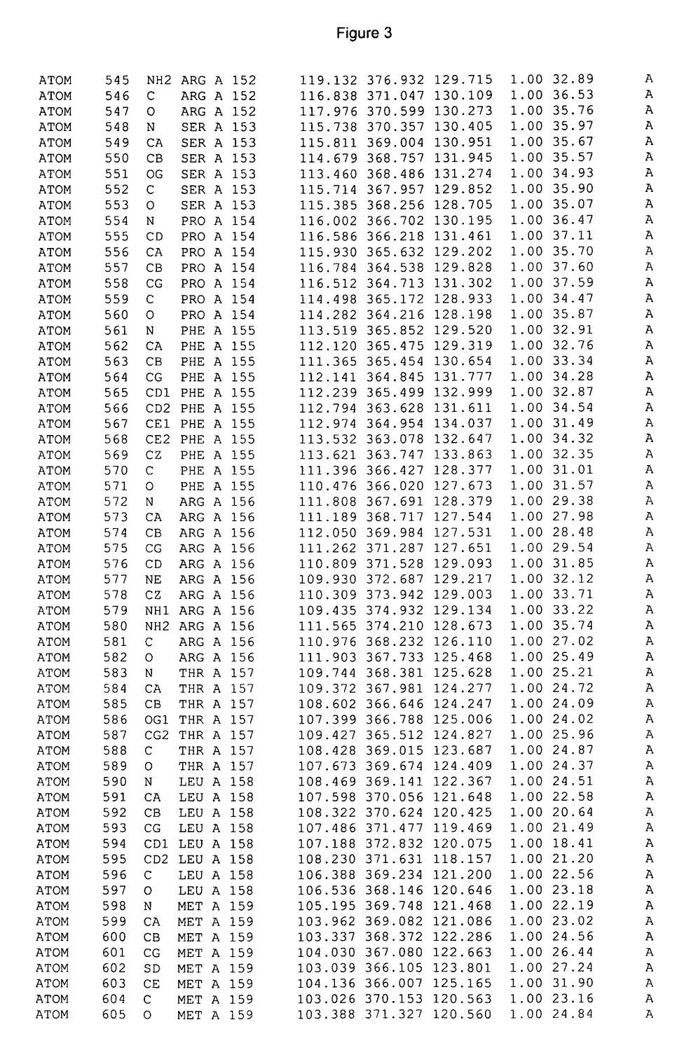

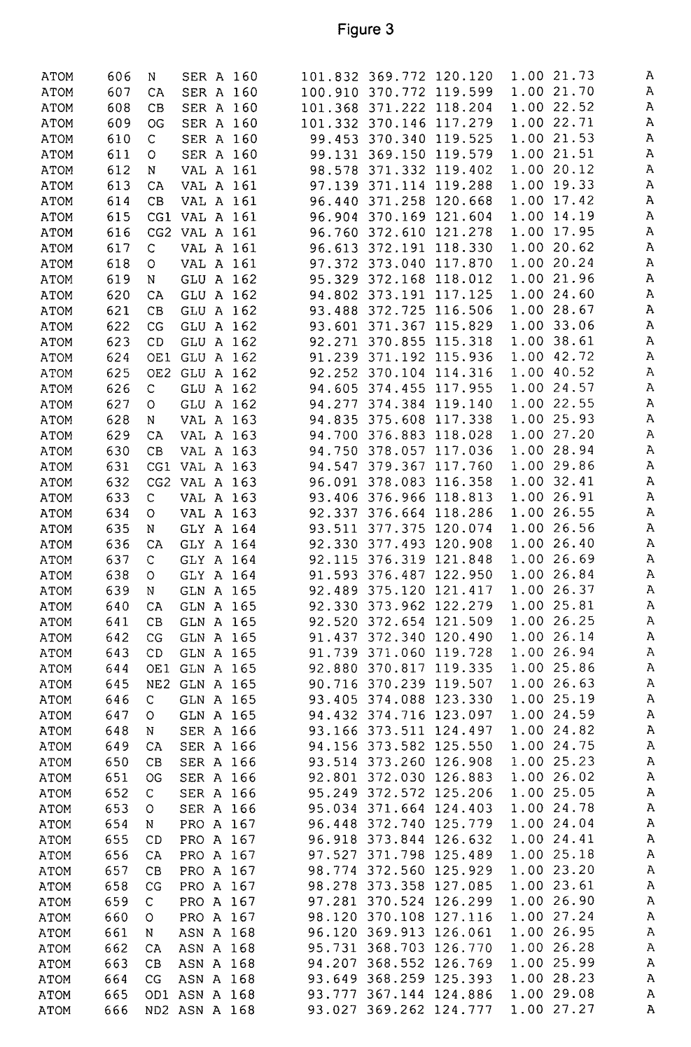

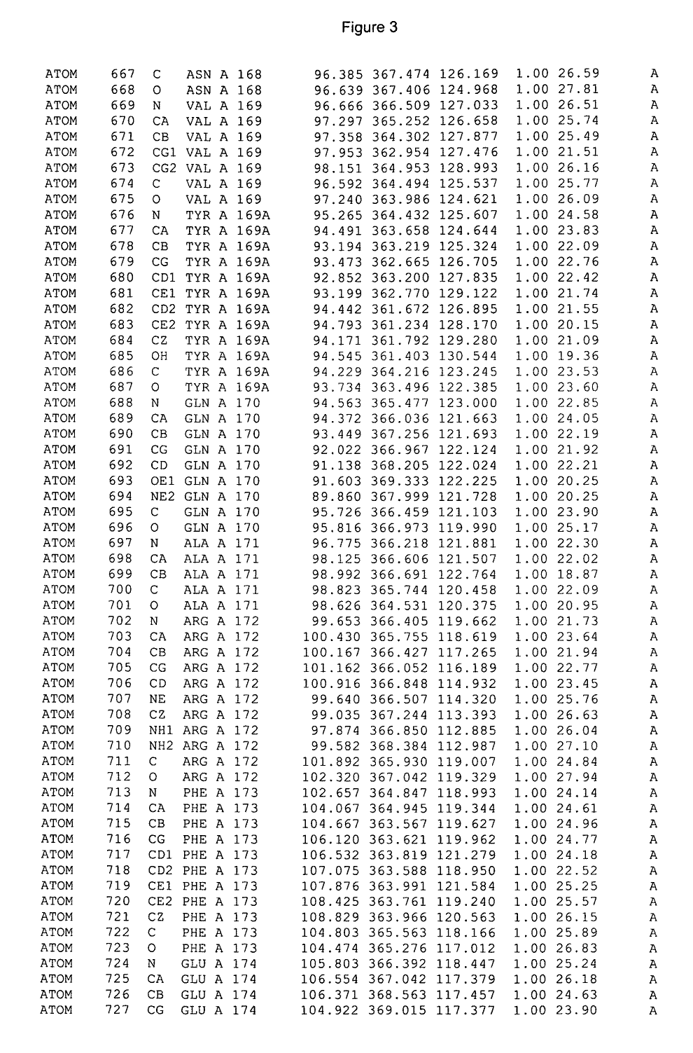

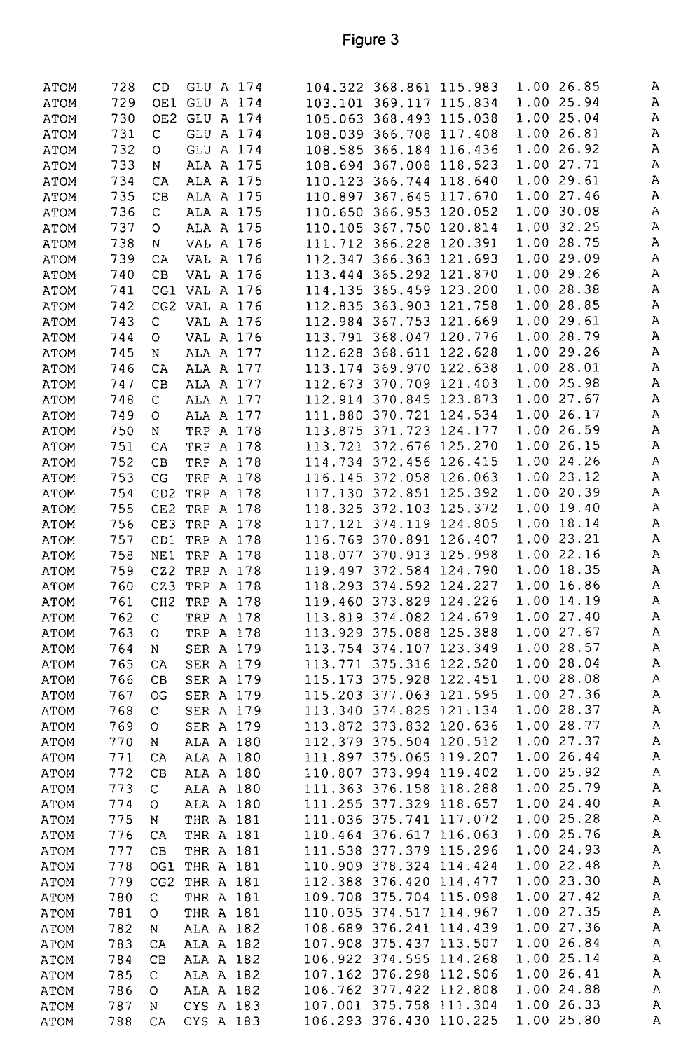

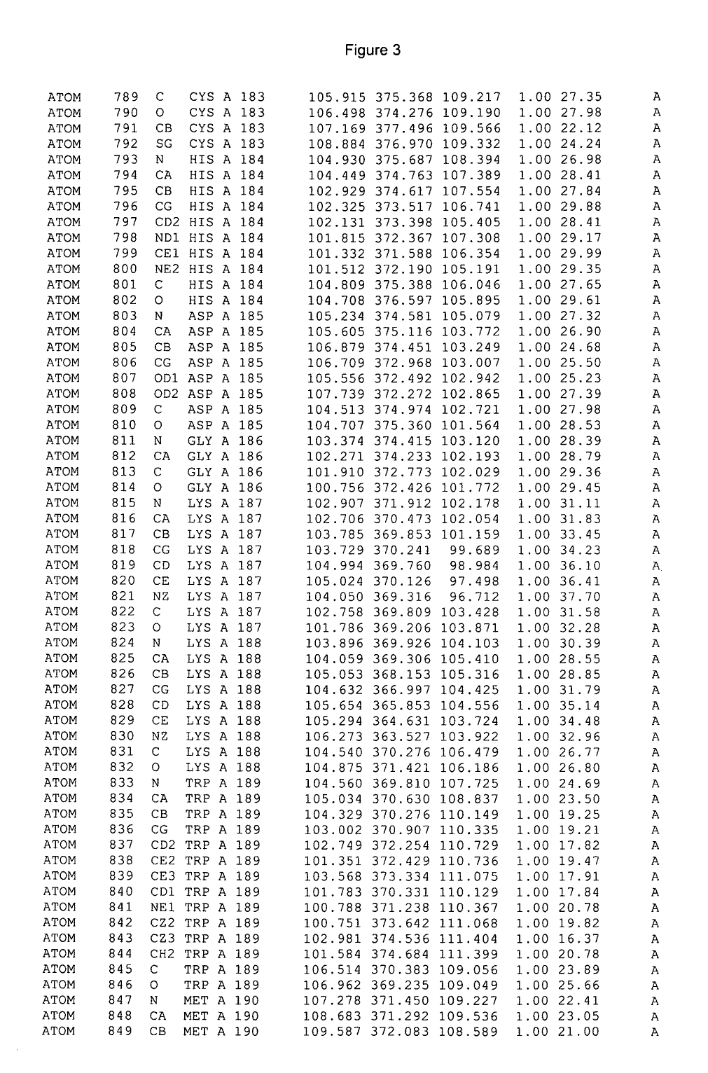

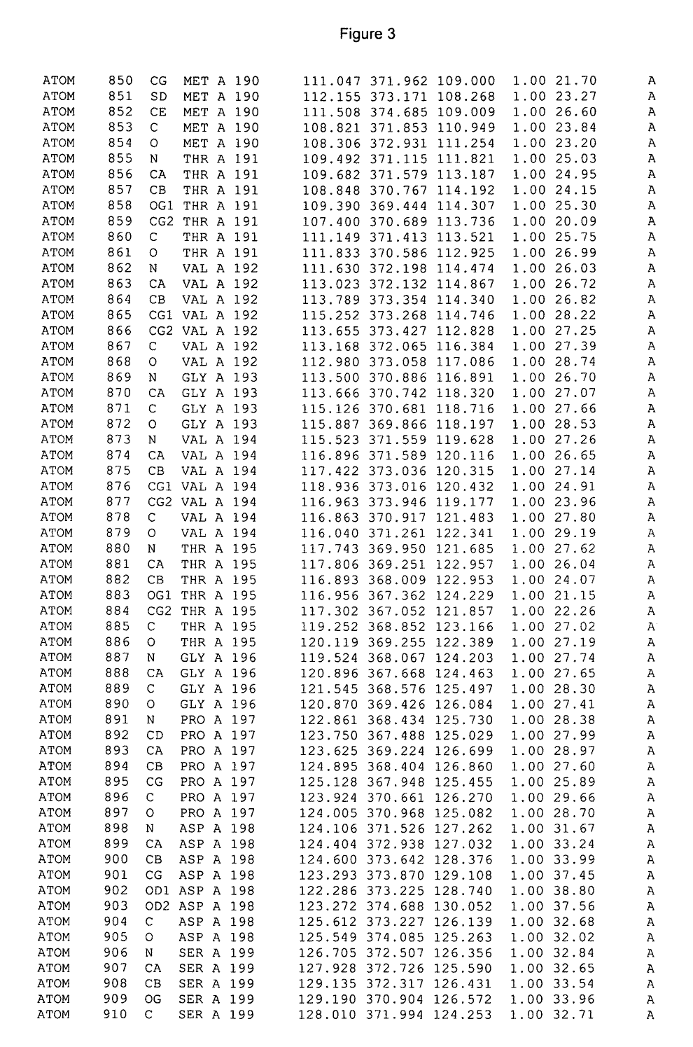

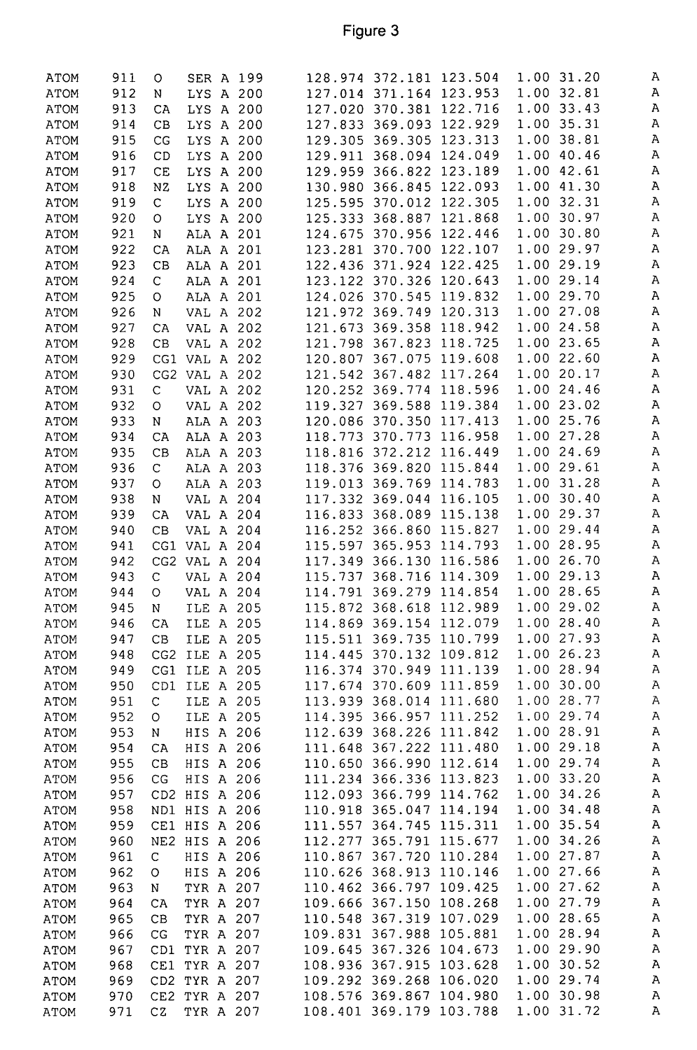

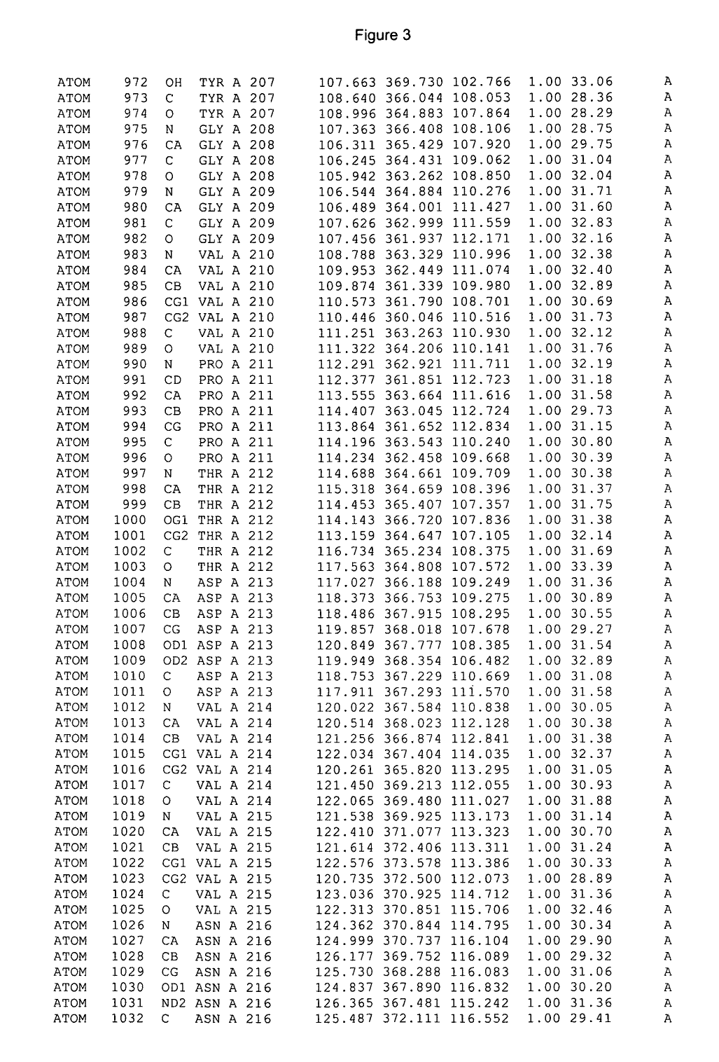

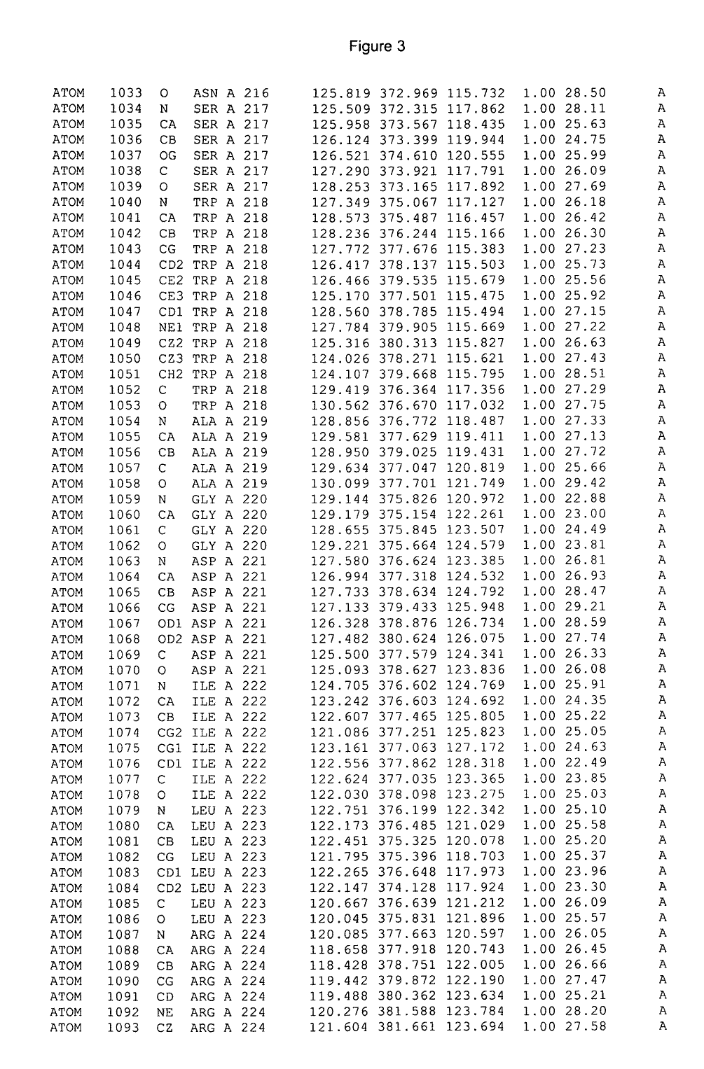

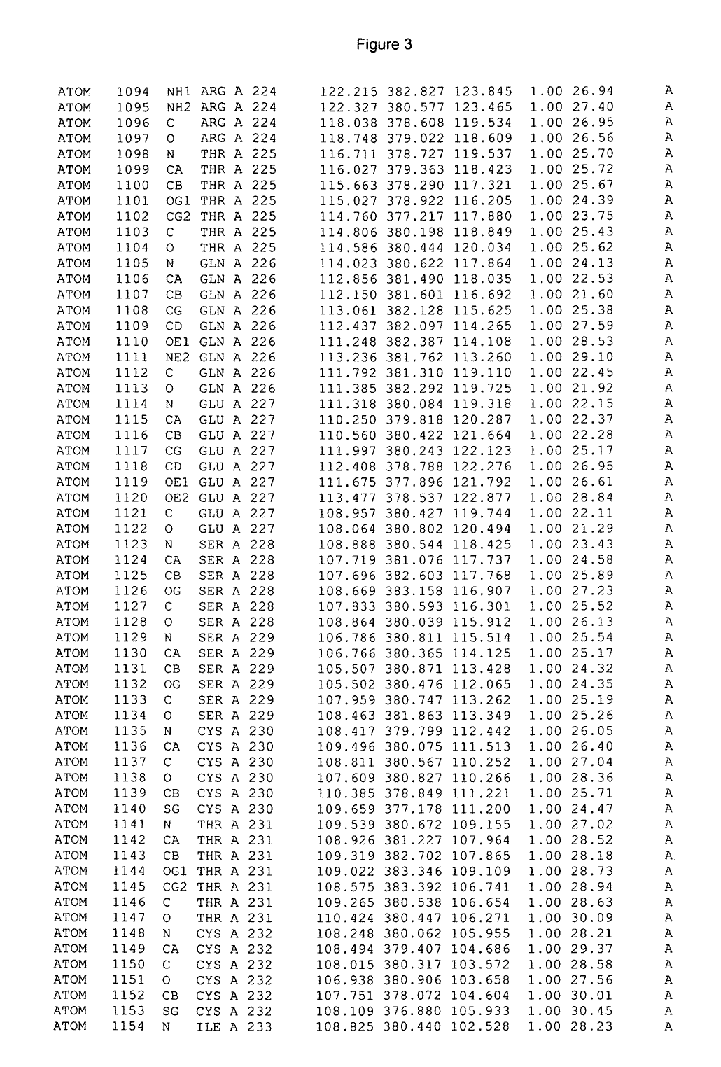

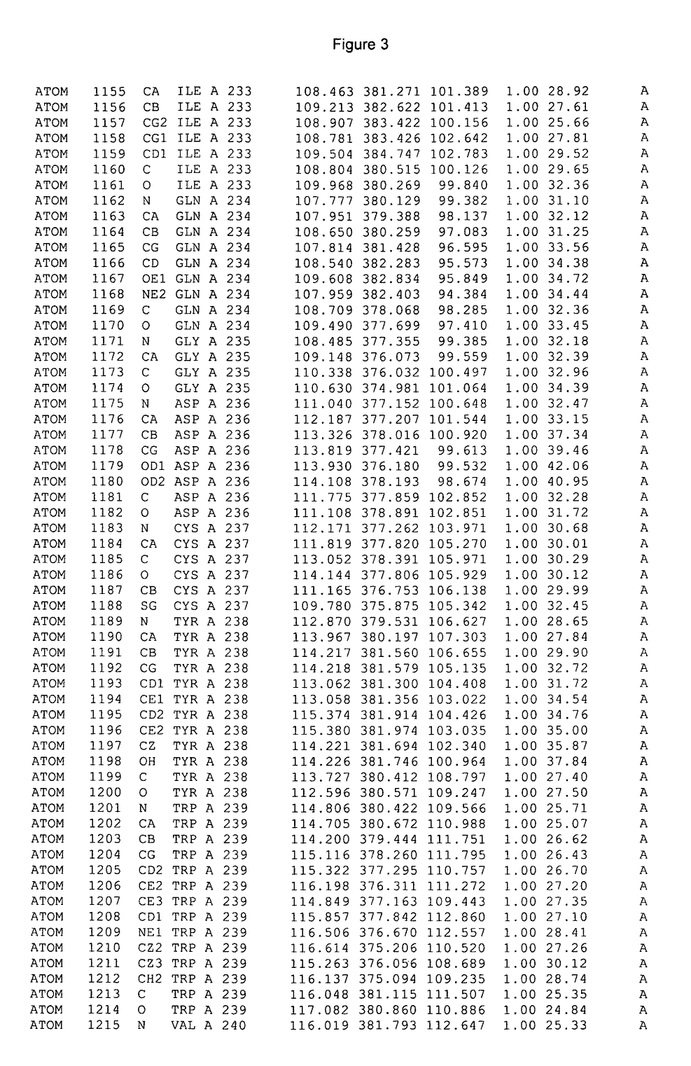

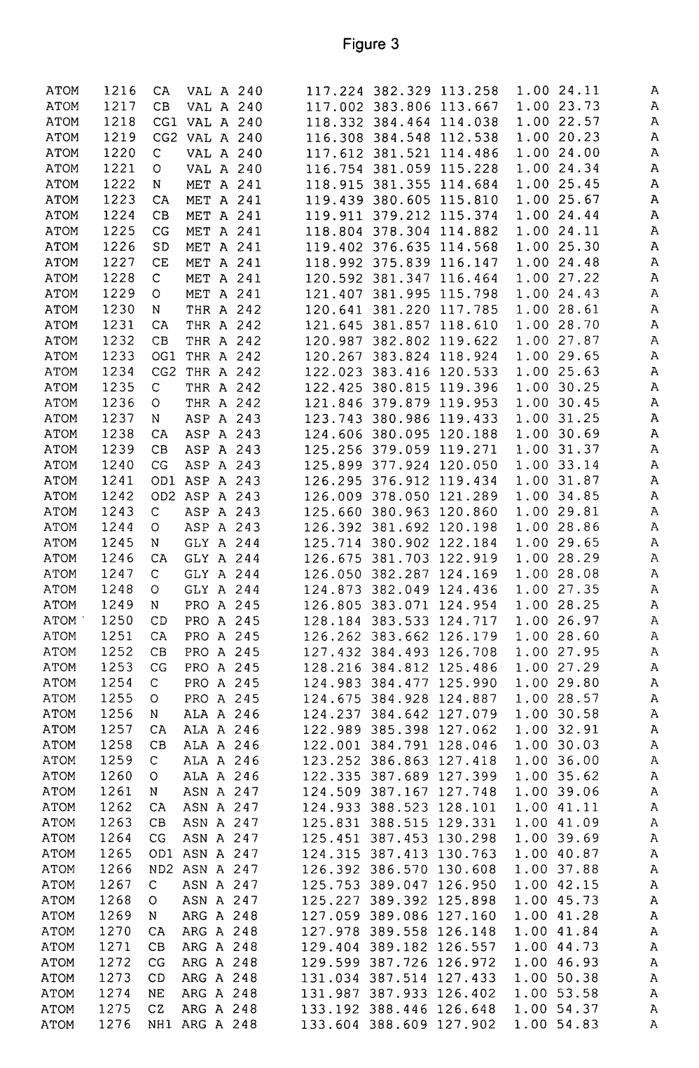

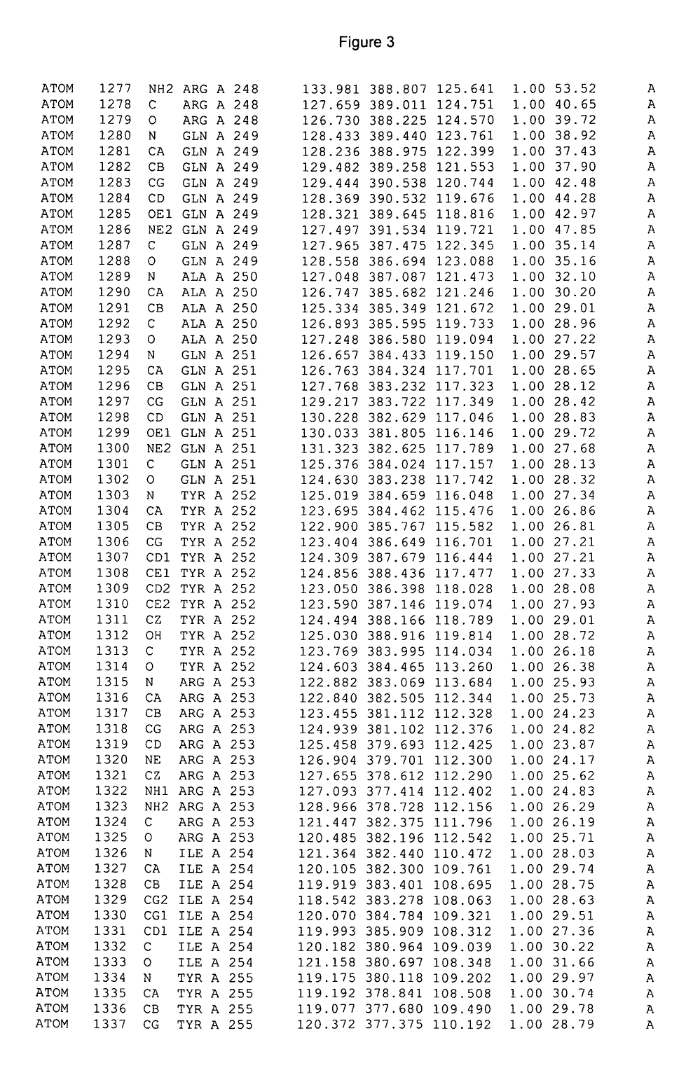

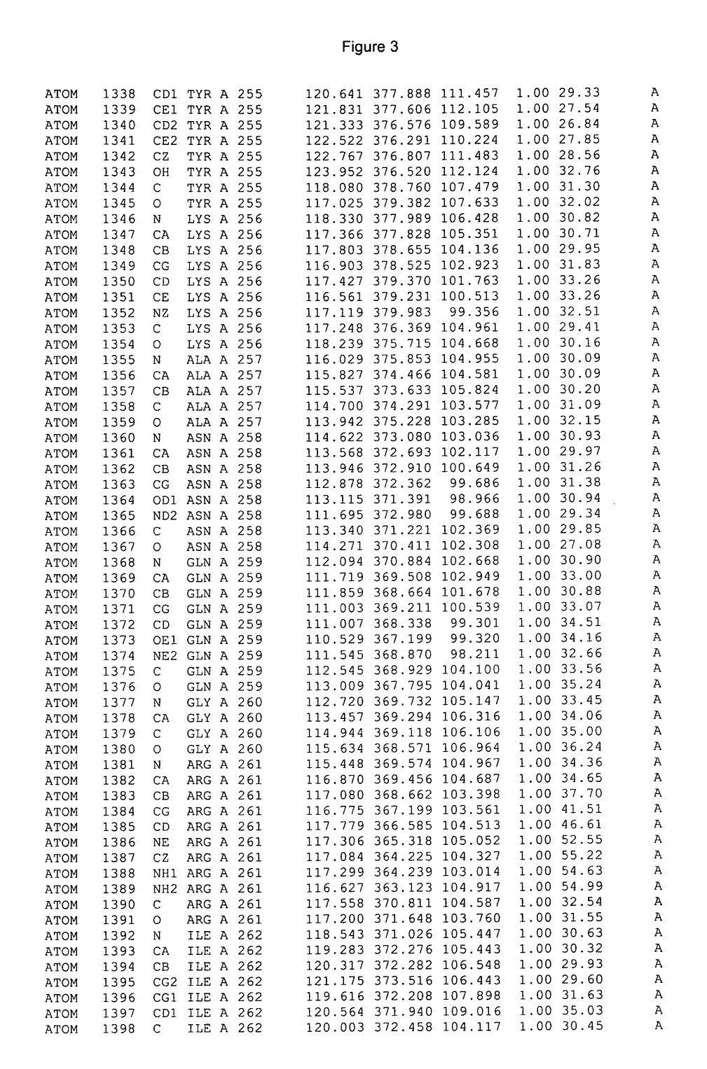

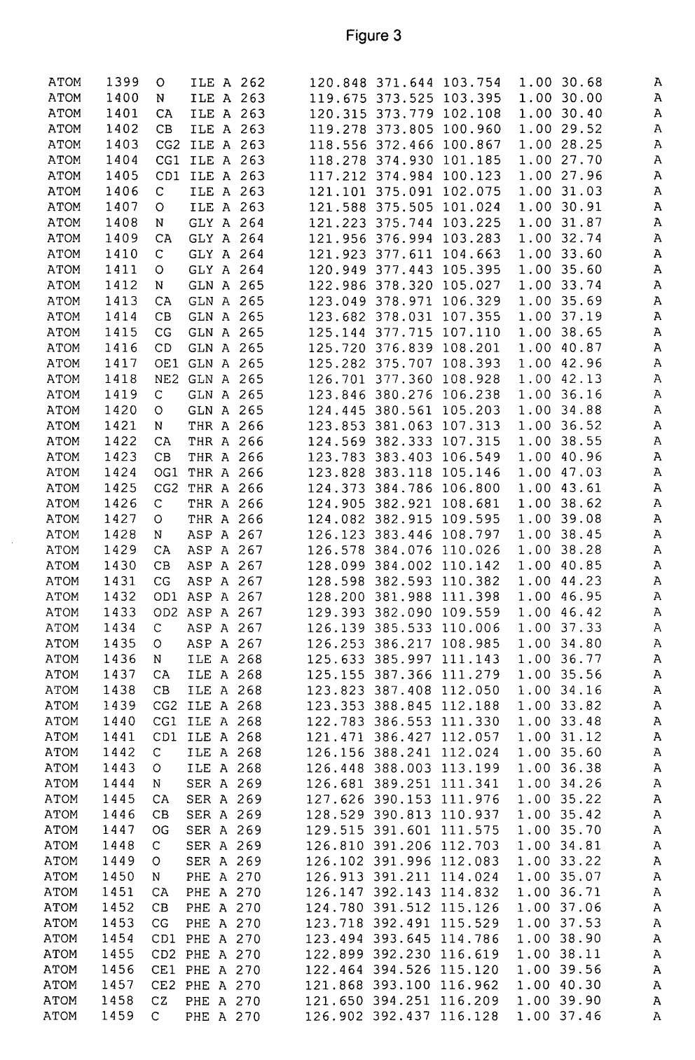

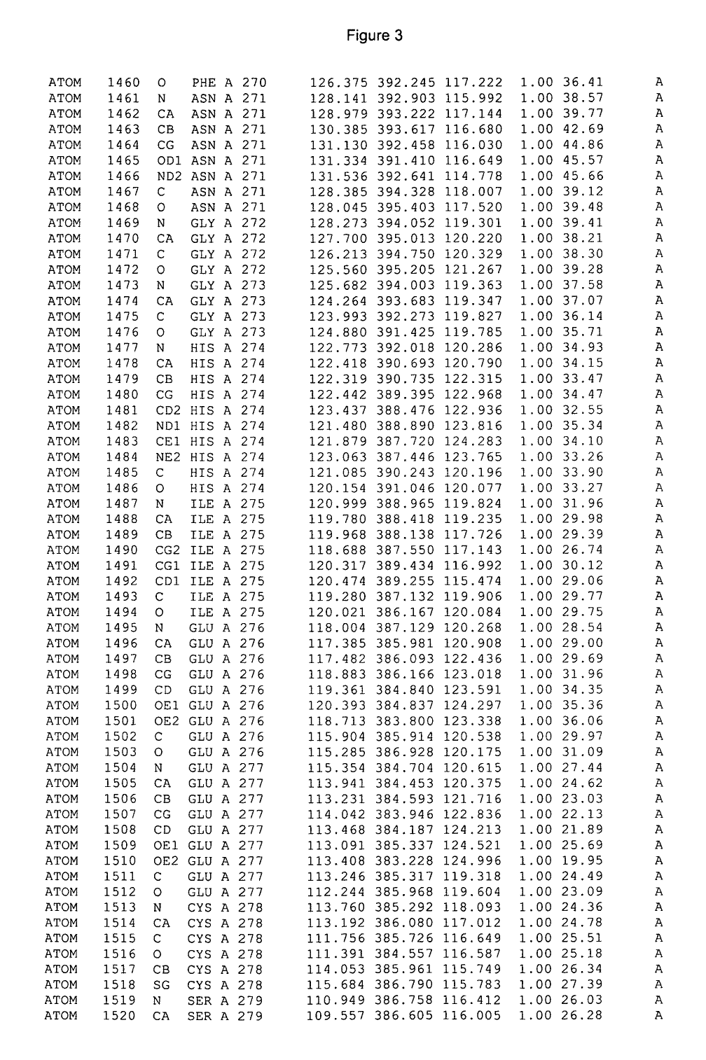

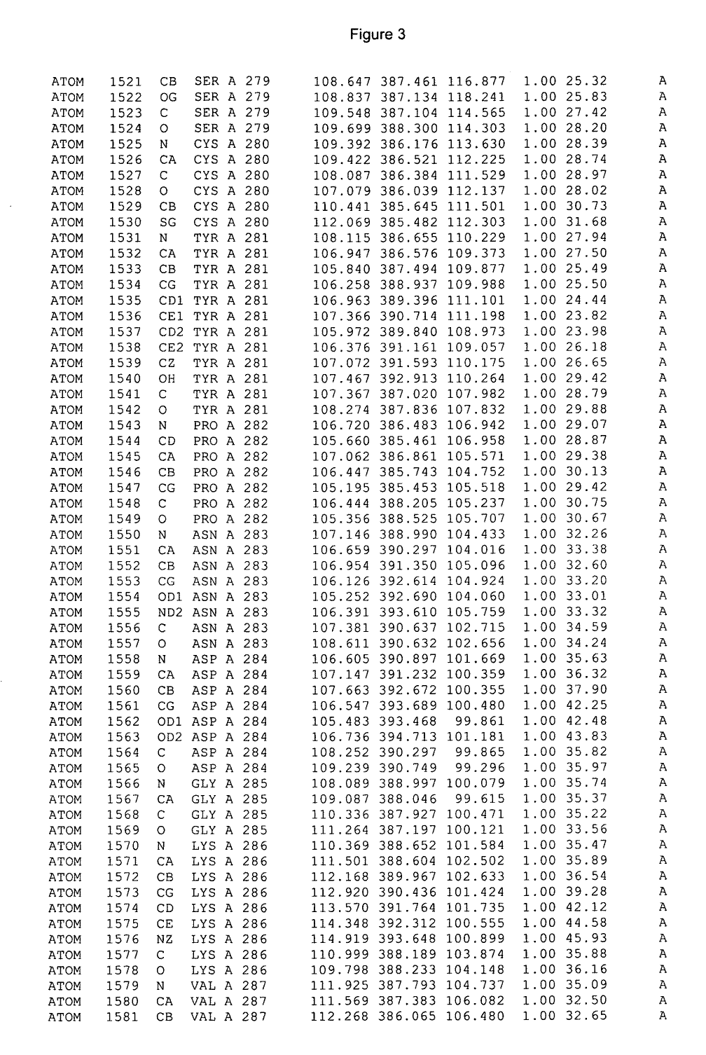

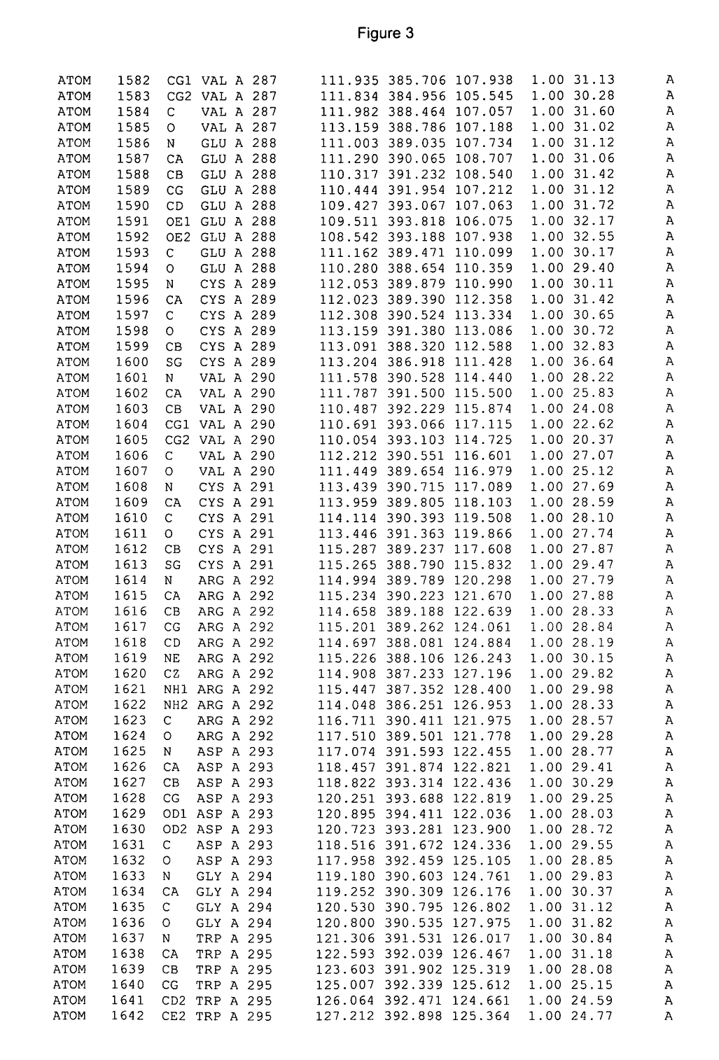

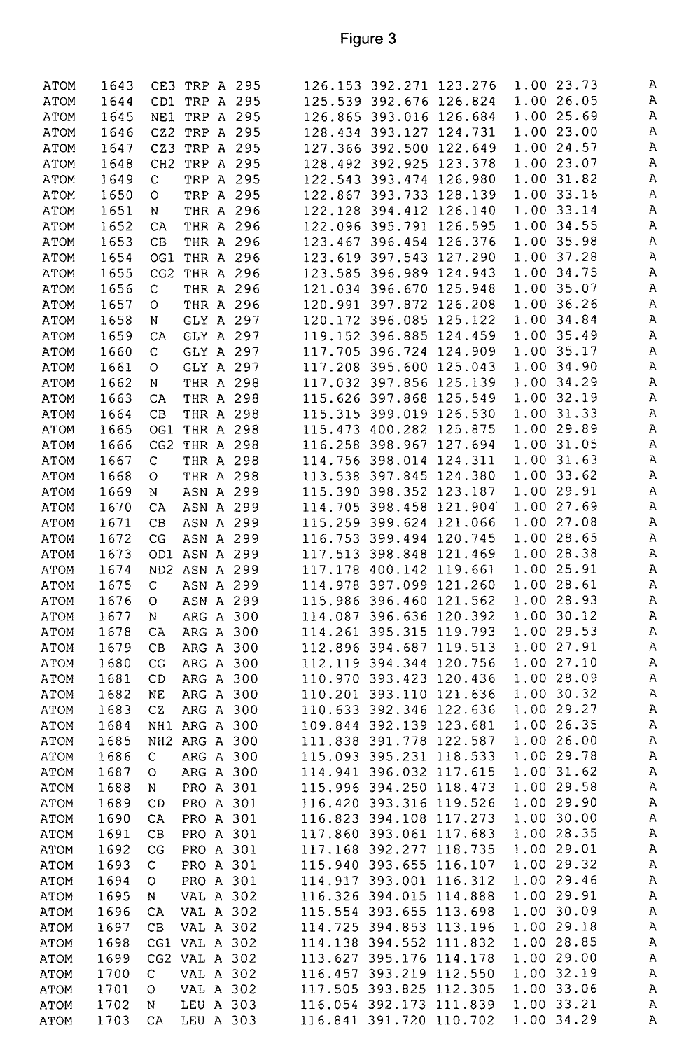

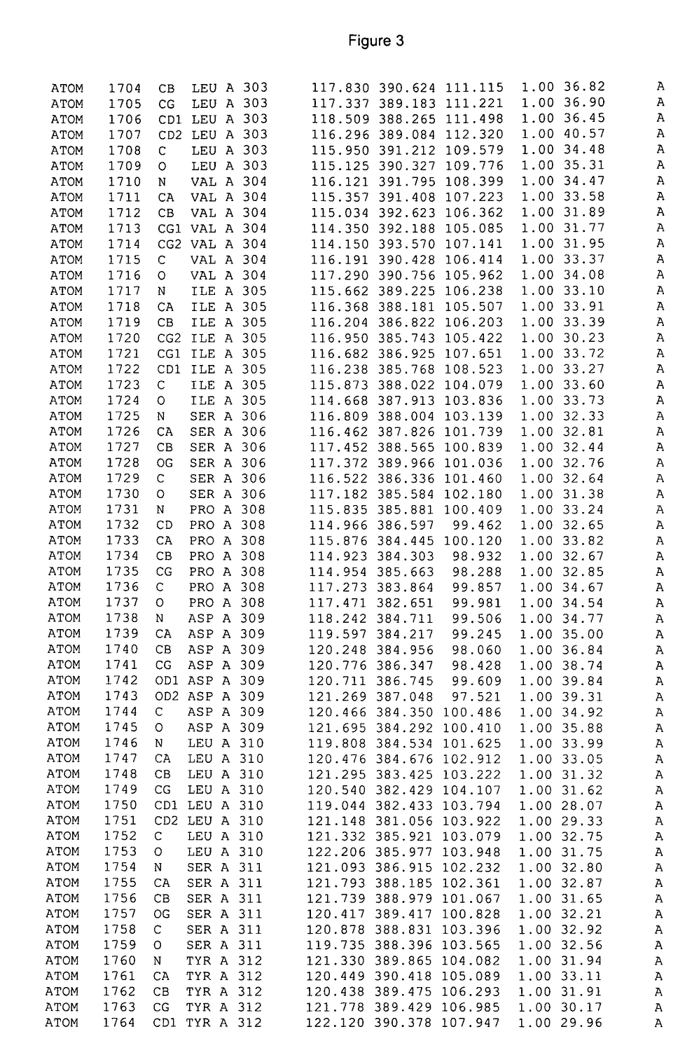

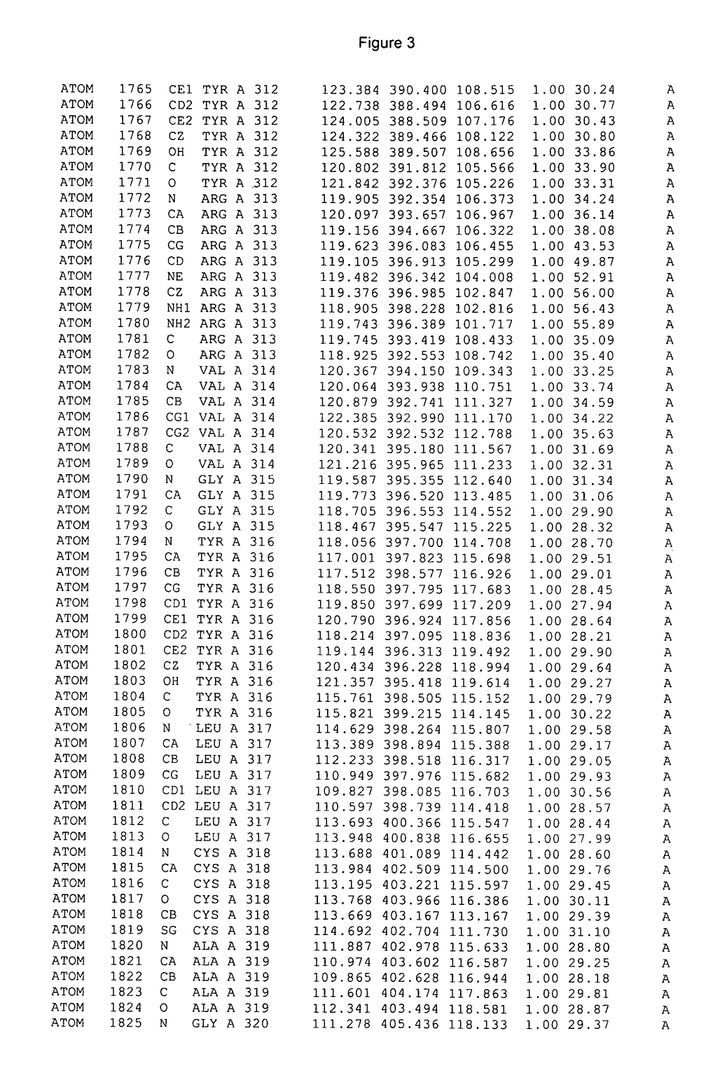

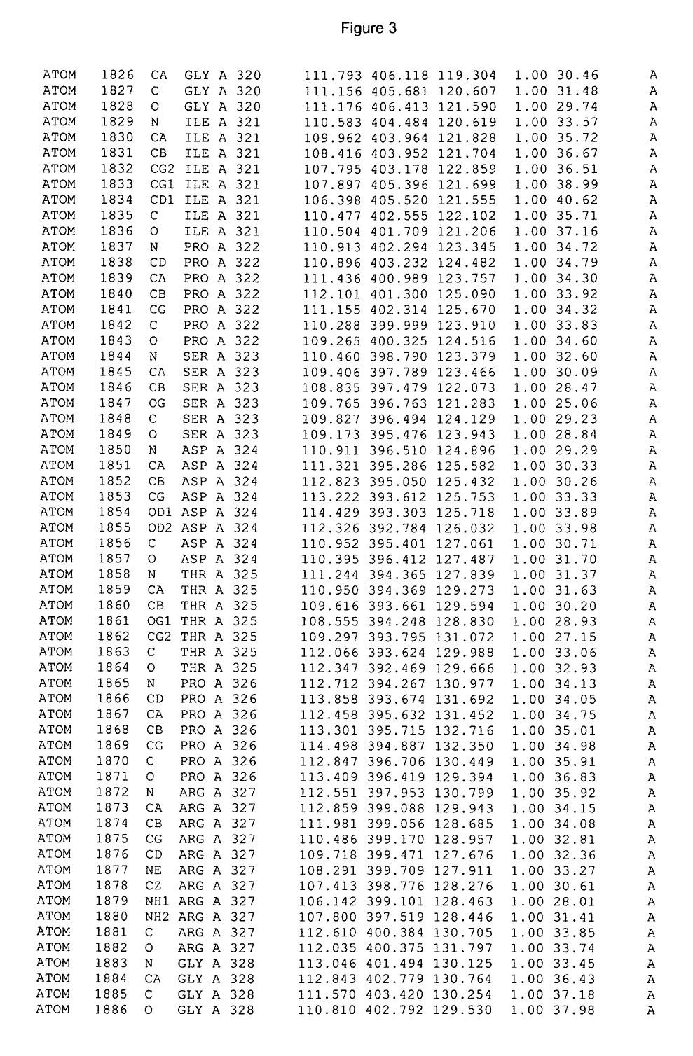

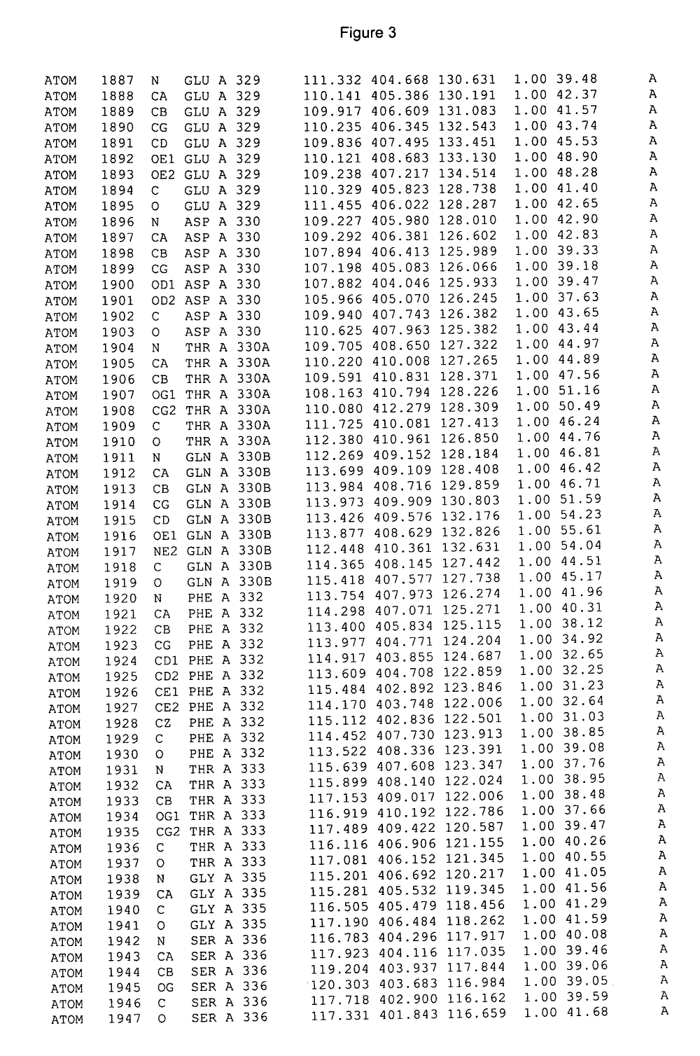

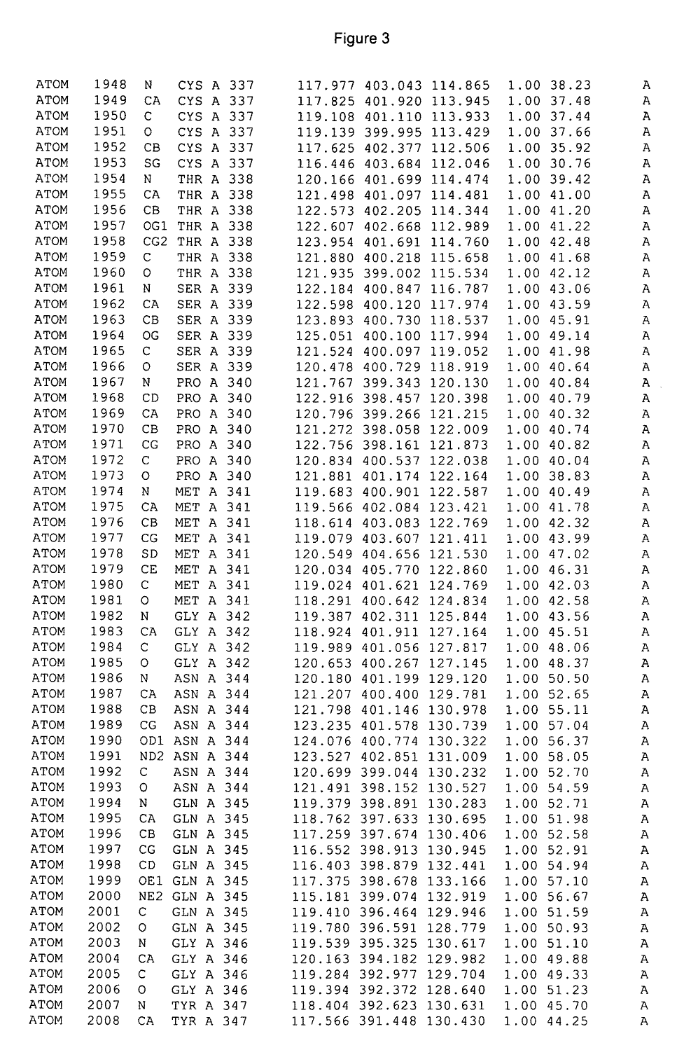

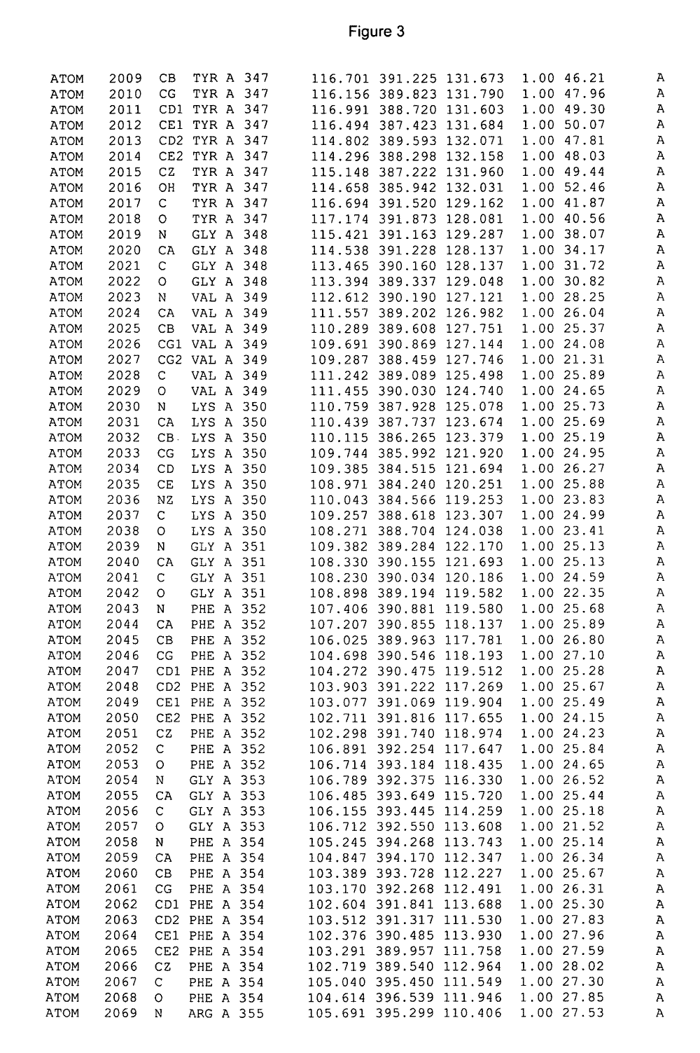

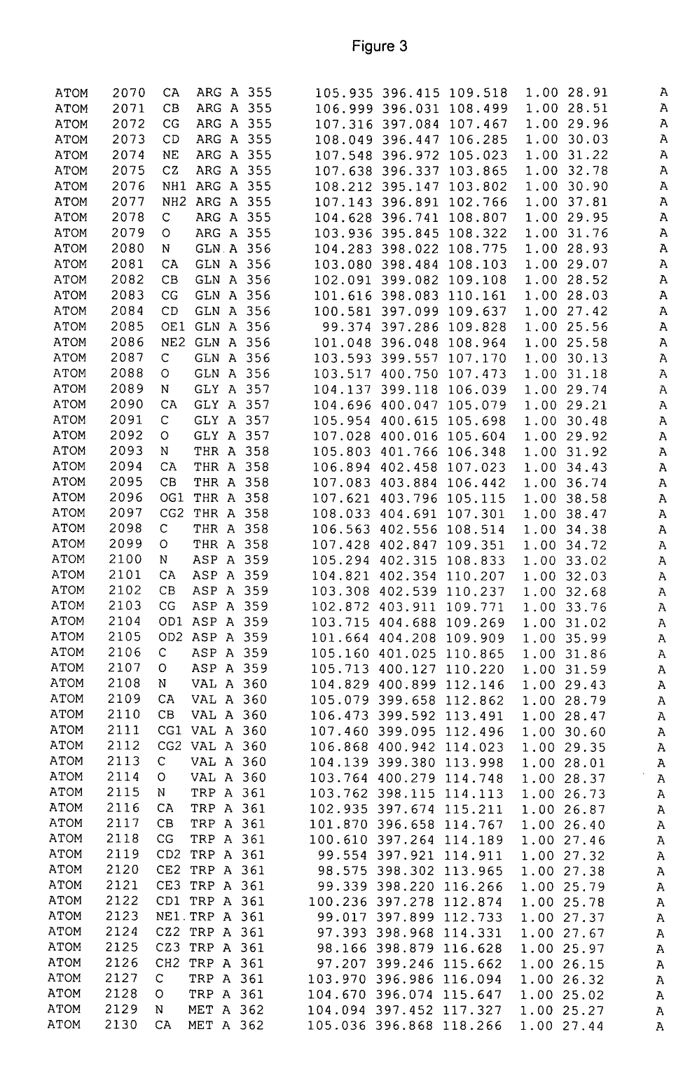

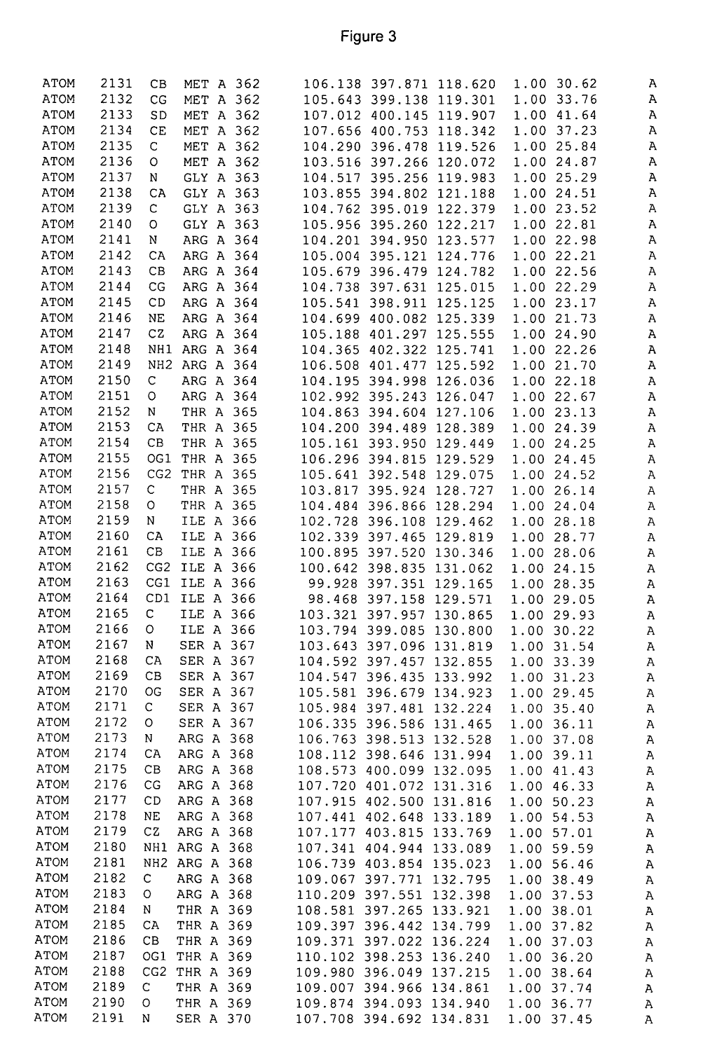

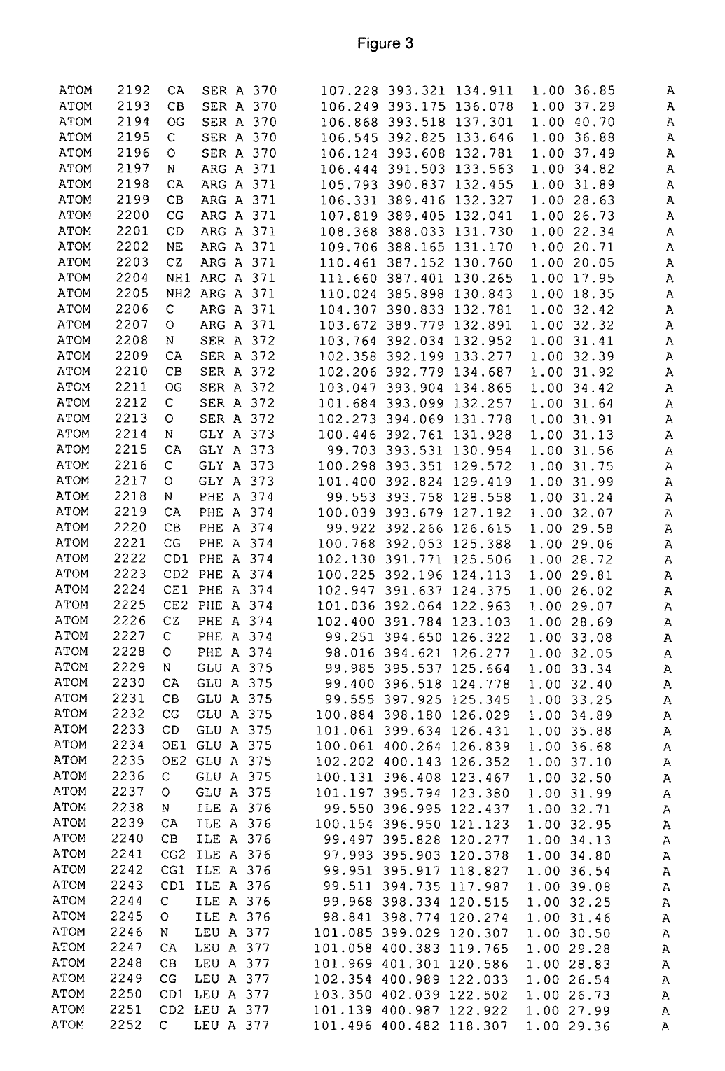

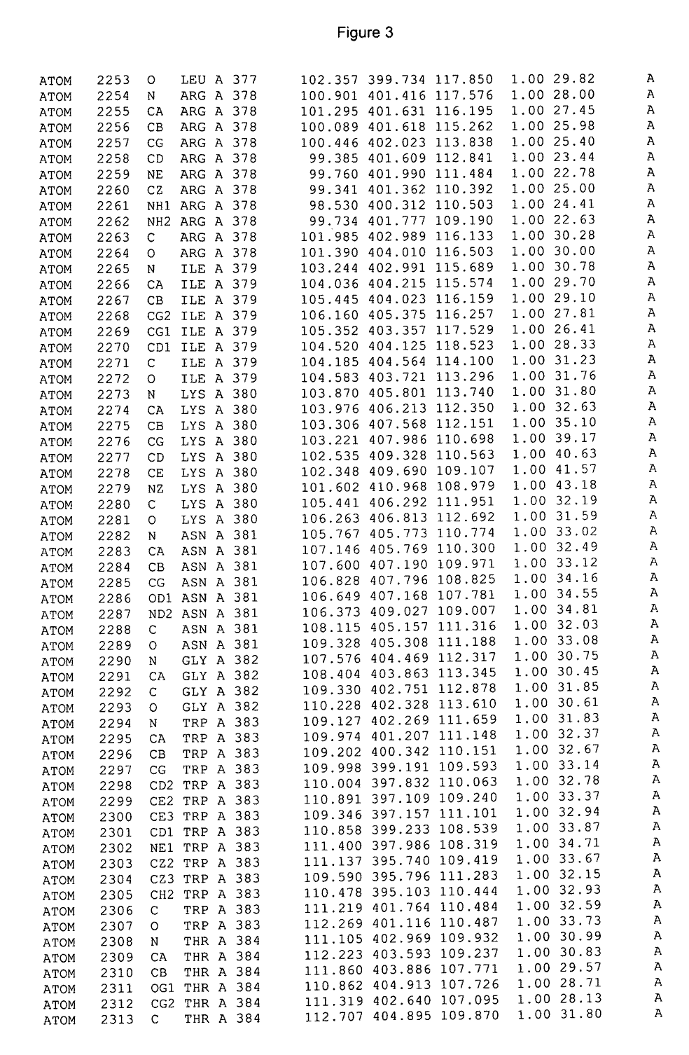

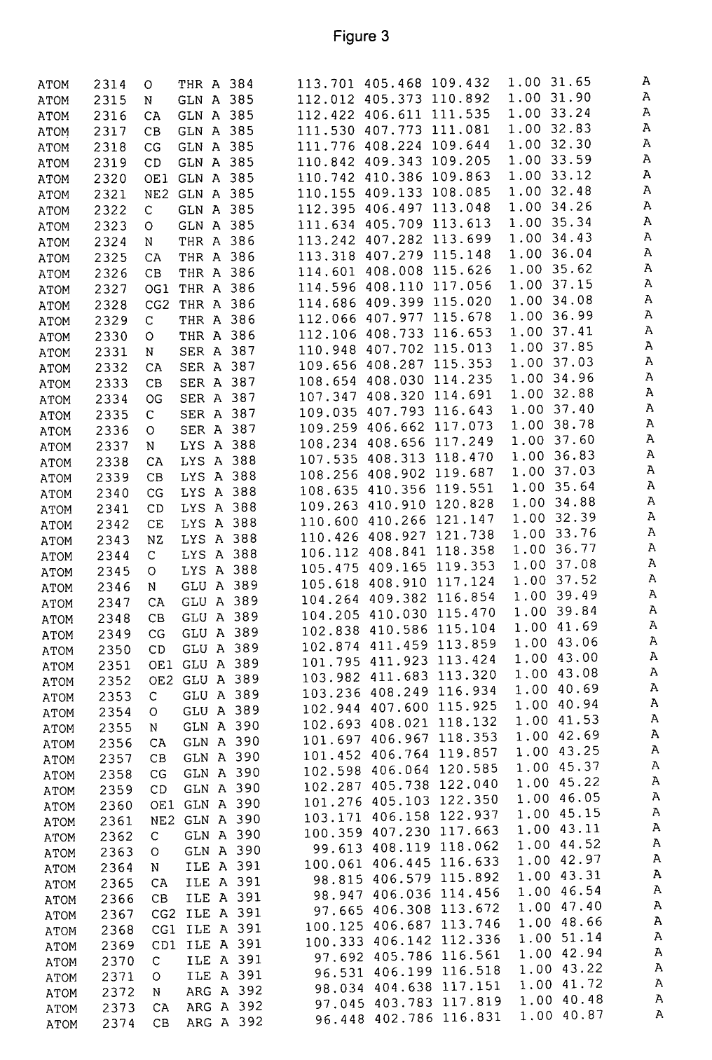

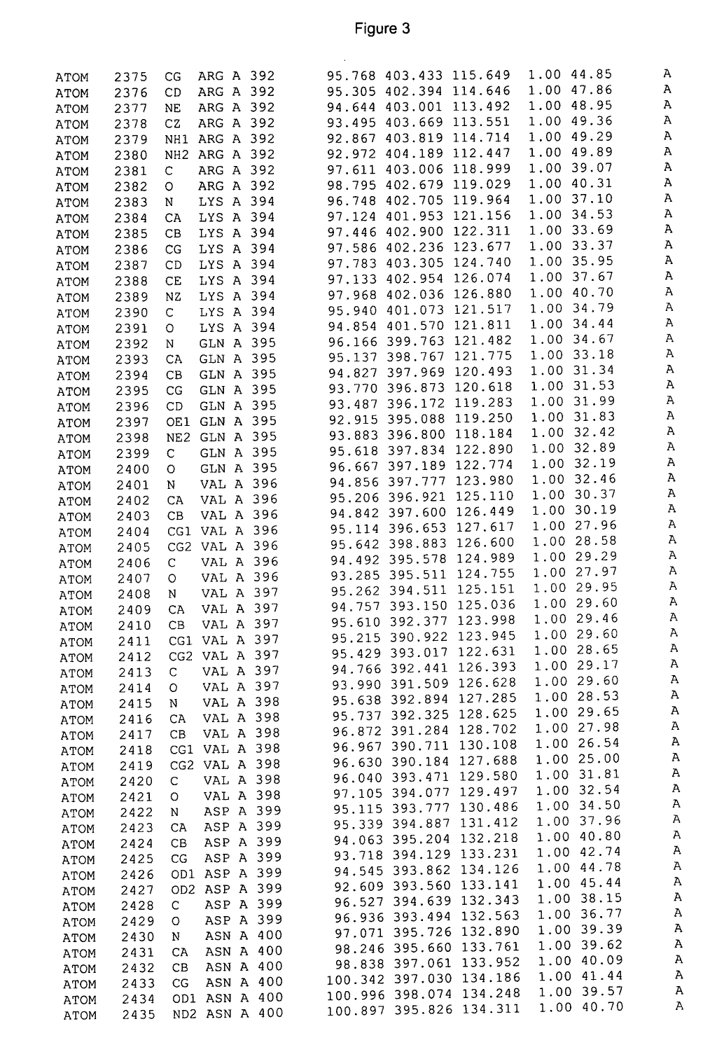

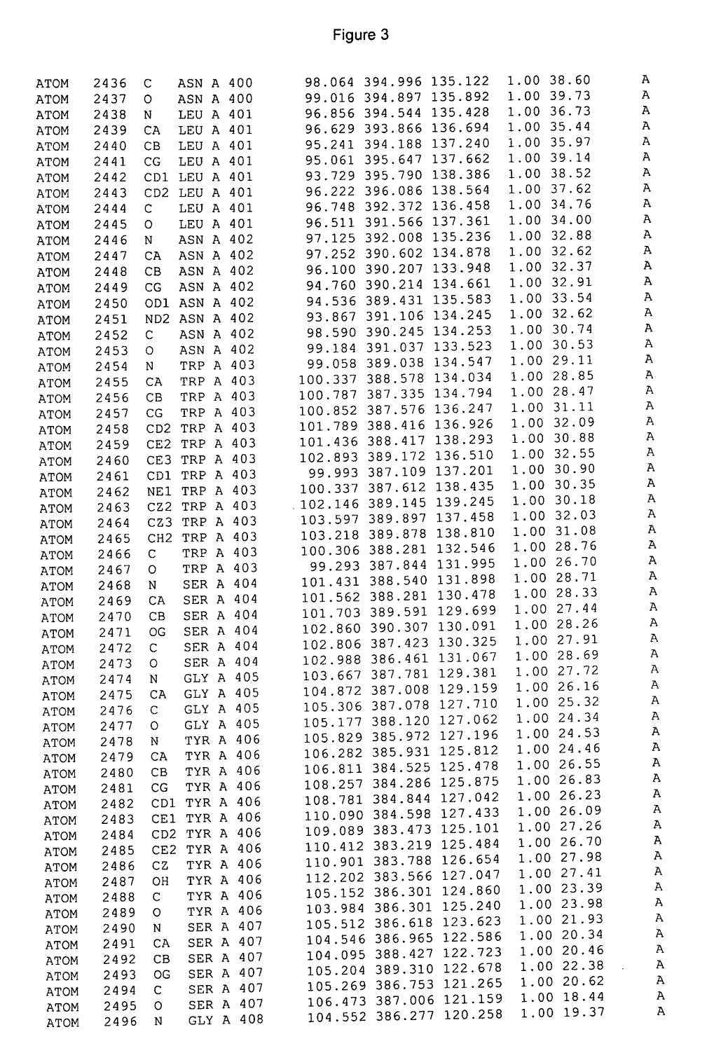

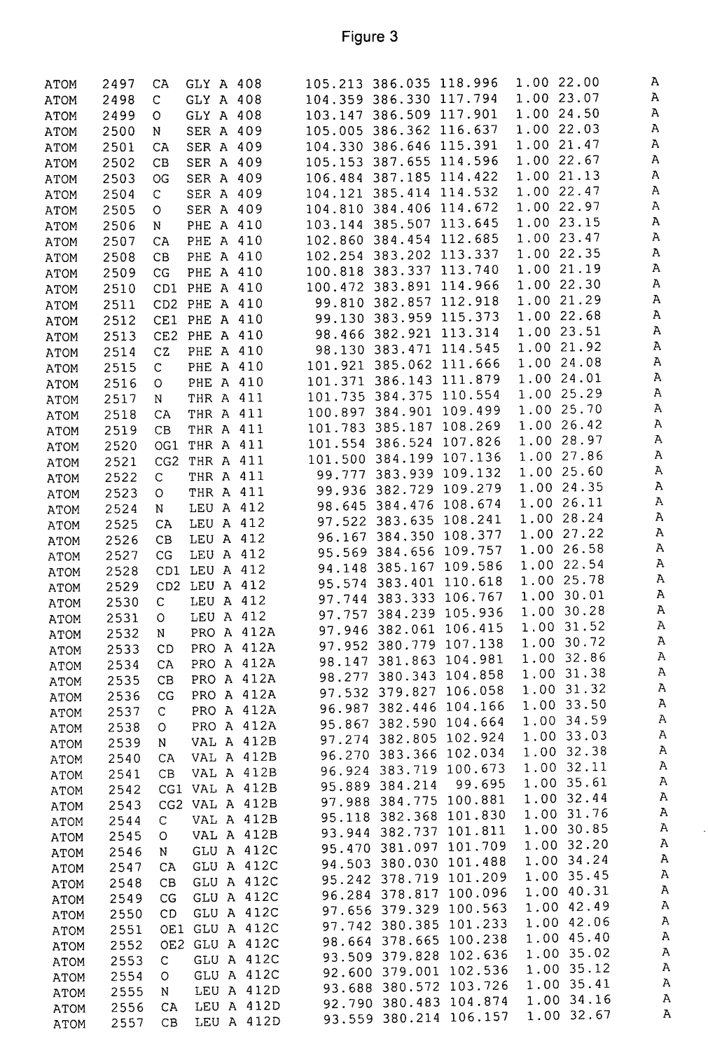

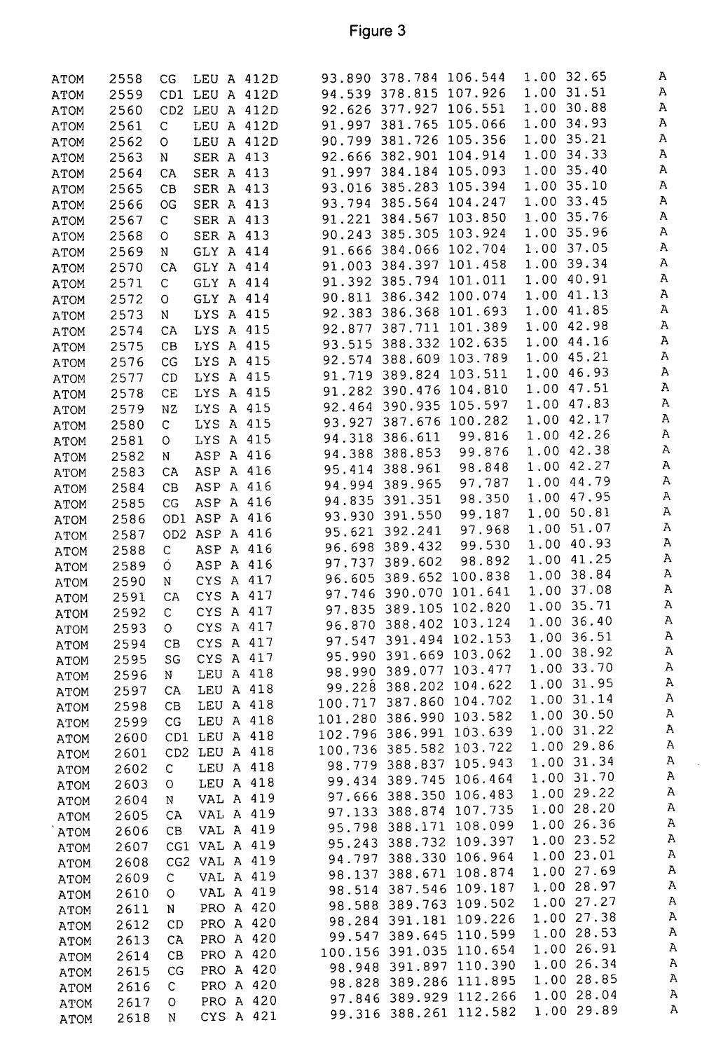

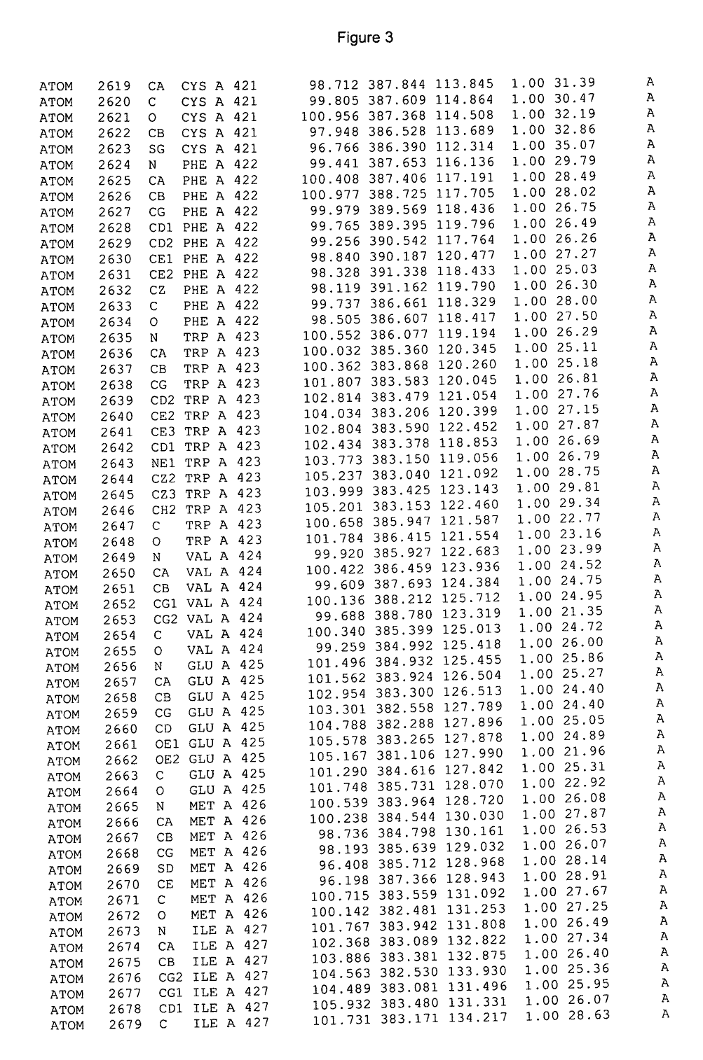

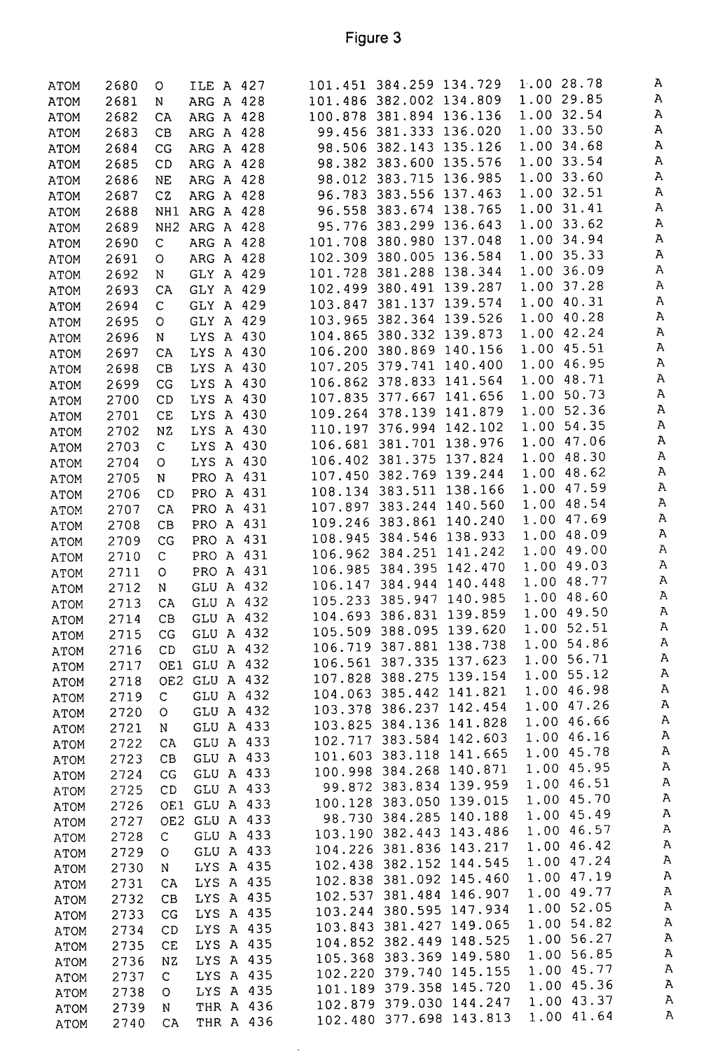

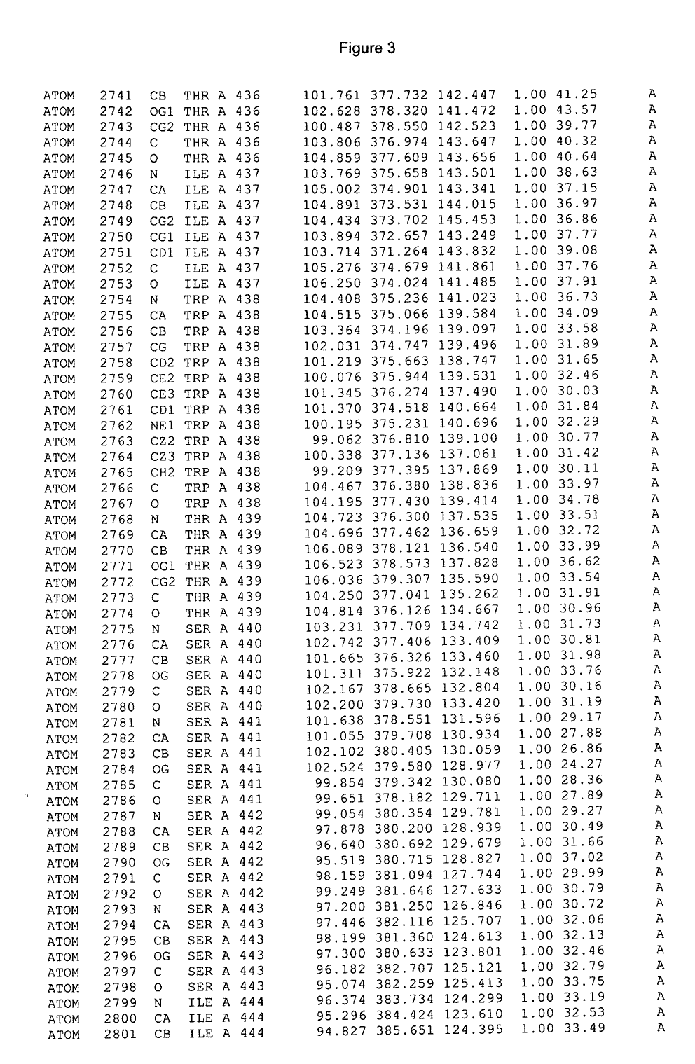

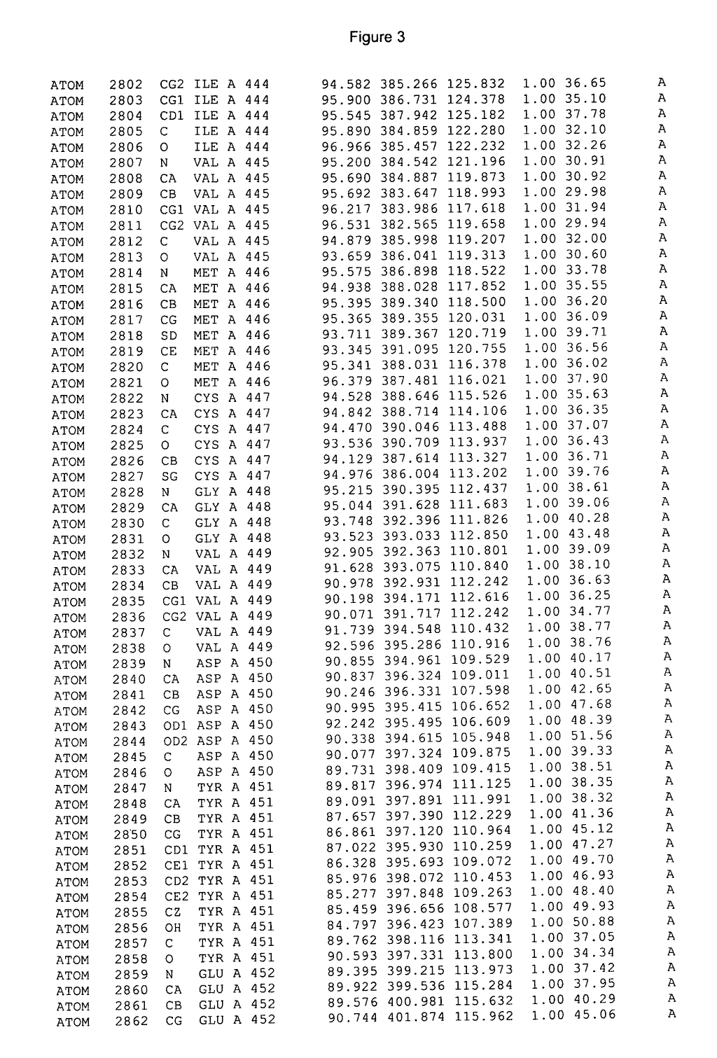

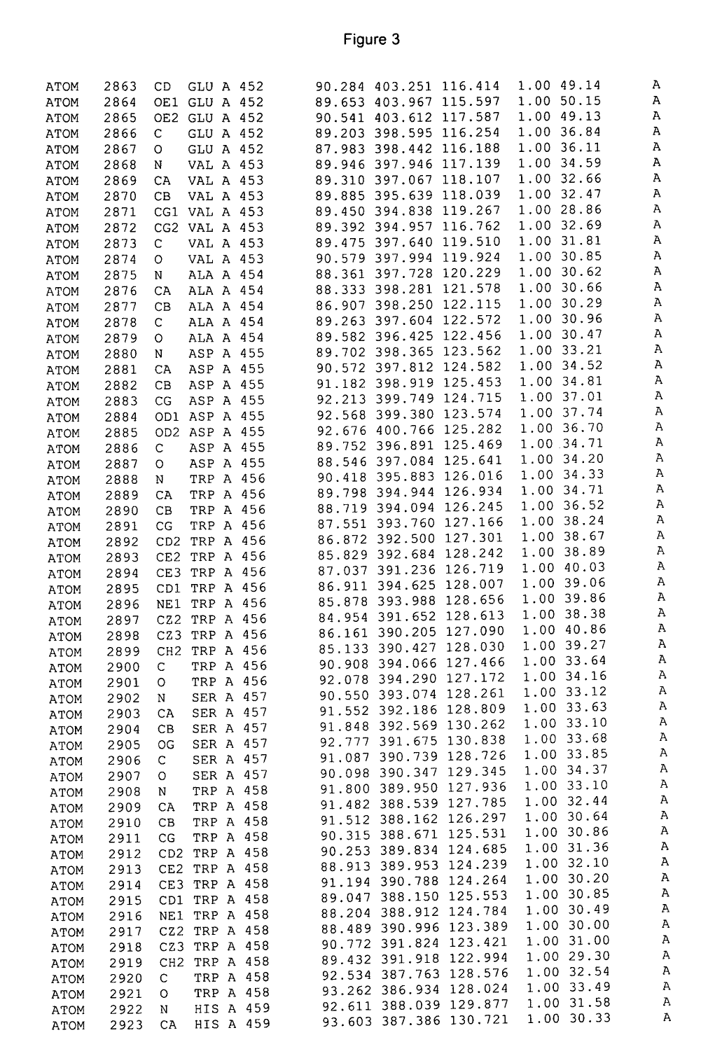

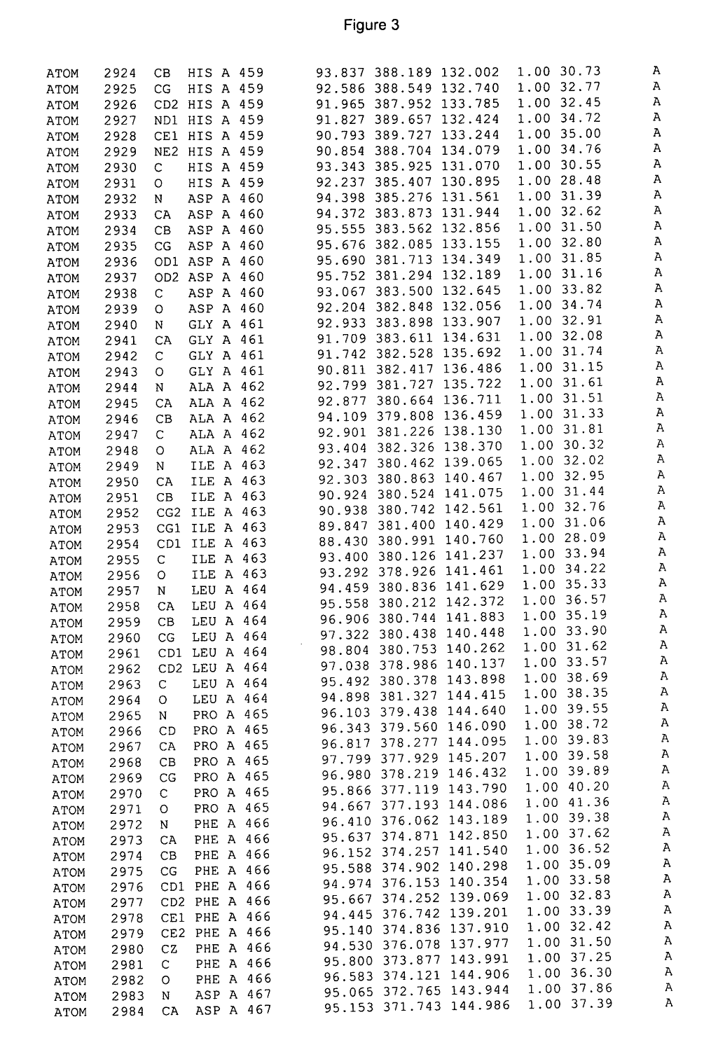

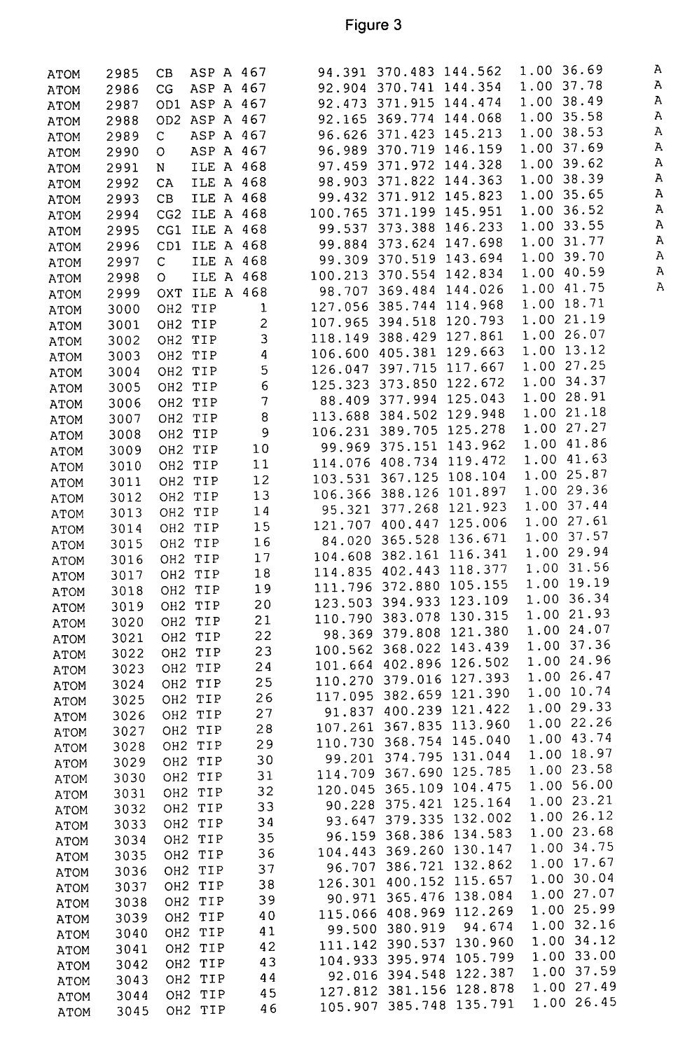

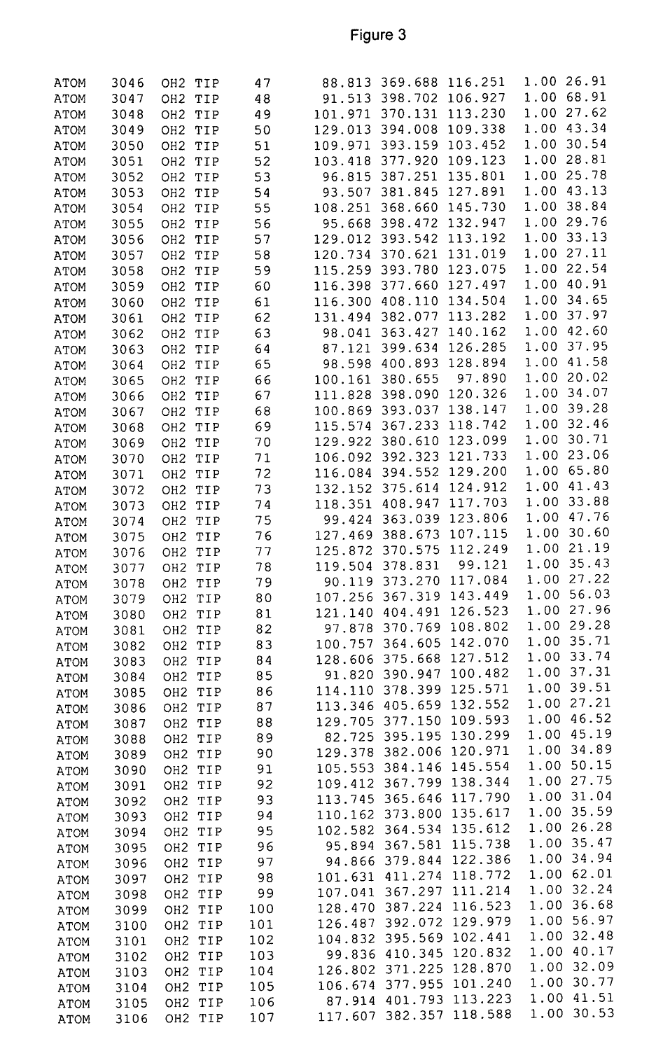

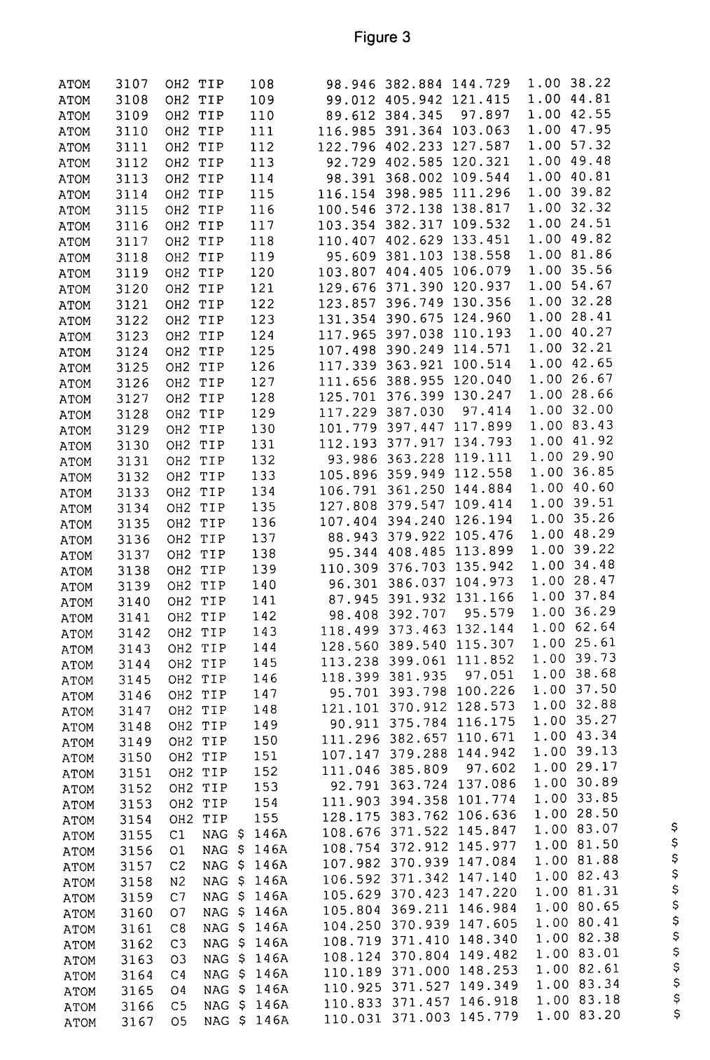

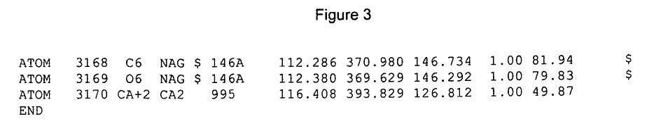

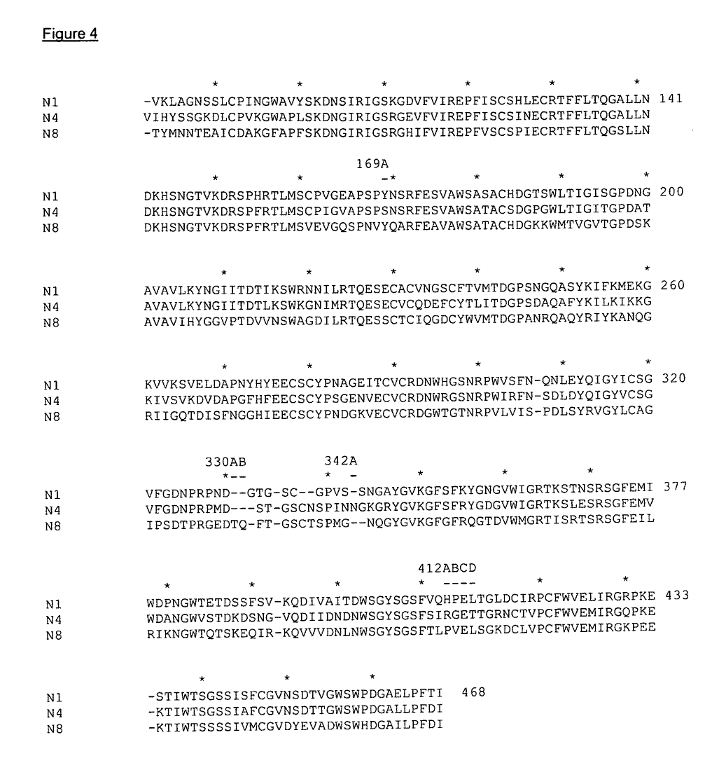

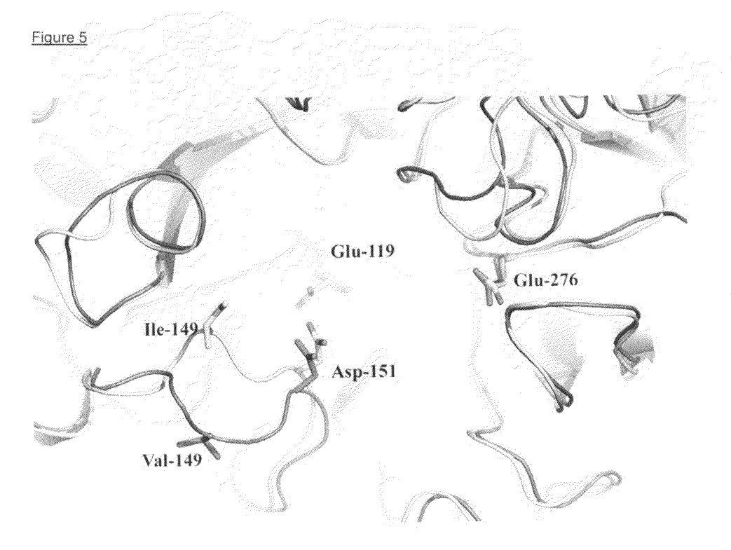

### BRIEF DESCRIPTION OF THE SEQUENCES

SEQ ID NO:1 is the sequence of the N1 neuraminidase used to produce a crystal. The protein is cleaved after position 61, such that the first amino acid of the crystal is Ser62 of SEQ ID NO:1.

SEQ ID NO:2 is the sequence of the N4 neuraminidase used to produce a crystal. The protein is cleaved after position 78, such that the first amino acid of the crystal is Ser79 of SEQ ID NO:2.

SEQ ID NO:3 is the sequence of the N8 neuraminidase used to produce a crystal. The protein is cleaved after position 72, such that the first amino acid of the crystal is Tyr73 of SEQ ID NO:3.

### DETAILED DESCRIPTION OF THE INVENTION

### A. Protein Crystals

The present invention provides a crystal of a N1 group neuraminidase protein. By “N1 group neuraminidase protein” it is meant a member of the N1 group of influenza virus A neuraminidase proteins. These are the N1, N4, N5 and N8 proteins.

The sequence of N1, N4 and N8 proteins from three strains of wild-type viruses are set out as SEQ ID NOs:1-3 respectively.

In order to produce crystals, the proteins are isolated by means known as such in the art, which include cleavage of the protein from the surface of laboratory-grown virus as described in the accompanying examples. This results in cleavage of the N-terminal region of the protein, such that the actual portion of SEQ ID NOs:1-3 which are crystallized are as defined above in the section “Brief Description of the Sequences”.

Alternatively, the proteins may be produced recombinantly and processed in an analogous manner to provide for crystals of the same size and form.

Influenza A virus has an RNA genome and it is known that variants of the N1 to N4 proteins exist in nature. Modifications to the sequences of SEQ ID NO:1-3 may be made to reflect variants of the type found in nature and crystals of such variants are also within the scope of the present invention. It is known that for many proteins, a limited number of changes may be made to the primary amino acid sequence without substantially altering the ability of the protein to crystallize. Thus for example, from 1 to 10, such as from 1 to 7, e.g. up to 1, 2, 3, 4, 5 or 6 amino acid substitutions, deletions or insertions may be made to the crystal-forming portions of sequences of SEQ ID NOs:1-3 without altering the crystal size or form, or without substantial alteration of the conditions under which the crystals form.

This is particularly the case wherein such substitutions, deletions or insertions occur at a location which is not conserved between all three of the N1, N4 and N8 proteins as indicated in the alignment of FIG. 4 (i.e. is different in at least one of the three sequences). Thus, in one aspect the invention extends to crystals wherein one or more (preferably as defined above) substitution, deletion or insertion appears in the N1 group protein at a non-conserved position. Thus reference to a N1 group neuraminidase protein (and in particular an N1, N4 or N8 protein) will be understood to encompass such variation.

Crystals of the invention may be apo crystals or co-crystals of a N1 group neuraminidase protein as defined above with a ligand. Thus in a further aspect, the invention provides a co-crystal of a N1 group neuraminidase protein and a ligand, such as a compound selected from the group oseltamivir, zanamivir, DANA (2-deoxy-2,3-didehydro-N-acetylneuraminic acid) and peramivir, or derivatives thereof.

Alternatively the ligand could be a compound whose interaction with a N1 group neuraminidase protein unknown.

Such co-crystals may be obtained by co-crystallization or soaking. Methods for the production of crystals and co-crystals are illustrated further in the accompanying examples.

The methodology described herein may be used generally to provide a N1 group neuraminidase protein crystal resolvable at a resolution of at least 3.0 Å, and preferably at least about 2.2 to 2.8 Å.

The invention thus further provides a N1 group neuraminidase protein crystal having a resolution of at least 3.0 Å, preferably at least 2.8 Å.

In a more particular embodiment, the invention provides a crystal of N1 having an C-orthorhombic space group C222₁, and unit cell dimensions a=201.74, b=201.51, c=212.43, with a unit cell variability of 5% in all directions.

In another embodiment, the invention relates to a co-crystal of an N1 and a ligand having the above-mentioned space group and unit cell dimensions. A particular embodiment of such a co-crystal is N1 with oseltamivir having an C-orthorhombic space group C222₁, and unit cell dimensions a=200.62, b=200.70, c=210.48.

The N1 protein is preferably that of residues 62-449 of SEQ ID NO:1 or a variant thereof having 1 to 10 amino acid substitutions, deletions or insertions.

In another embodiment, the invention provides a crystal of N4 having an cubic space group P432 and unit cell dimensions a=b=c=193.79 Å, with a unit cell variability of 5% in all directions.

In a further embodiment, the invention provides a co-crystal of N4 and a ligand having a having an cubic space group I432 and unit cell dimensions a=b=c=193.44 Å, with a unit cell variability of 5% in all directions. A particular ligand is DANA.

The N4 protein may be that of residues 79-470 of SEQ ID NO:2 or a variant thereof having from 1 to 10 amino acid substitutions, deletions or insertions.

In another aspect, the invention provides a crystal of N8 having a tetragonal space group I4 having unit cell dimensions a=b=90.67 Å, c=c=109.4 Å, alpha=90 beta=90 gamma=90, with a unit cell variability of 5% in all dimensions.

In another embodiment, the invention relates to a co-crystal of an N8 and a ligand having the above-mentioned space group and unit cell dimensions. Embodiments of such a co-crystal is N8 with DANA having a tetragonal space group I4 having unit cell dimensions a=b=90.38, c=111.49; N8 with oseltamivir having a tetragonal space group I4 having unit cell dimensions a=b=90.58, c=110.78 or a=b=90.42, c=109.71; and N8 with zanamivir having a tetragonal space group I4 having unit cell dimensions a=b=90.41, c=109.30; all said co-crystals having a unit cell variability of 5% in all dimensions.

There is also provided a co-crystal of N8 with peramivir having a tetragonal space group I4 having unit cell dimensions a=b=89.78, c=93.23 having a unit cell variability of 5% in all dimensions.

The N8 protein may be that of residues 73-470 of SEQ ID NO:3 or a variant thereof having from 1 to 10 amino acid substitutions, deletions or insertions.

### B. Crystal Coordinates

In a further aspect, the invention also provides a crystal of a N1 group neuraminidase protein having the three dimensional atomic coordinates from any one of Tables 1 to 3.

An advantageous feature of the structures defined by the atomic coordinates of Tables 1 and 1 to 3 are that they have a resolution better than about 3.0 Å.

A further advantage of the a N1 group neuraminidase protein structures of Tables 1 to 3 are that they are unliganded, apo structures. This makes them particularly suitable for soaking in ligands and hence determining co-complex structures and is also ideal for modelling purposes as there is no conformational bias from a ligand.

Tables 1 to 3 give atomic coordinate data for the N1 group neuraminidase proteins N1, N4 and N8 respectively. In these Tables the third column denotes the atom, the fourth the residue type, the fifth the chain identification, the sixth the residue number (the atom numbering is with respect to the alignment of FIG. 4), the seventh, eighth and ninth columns are the X, Y, Z coordinates respectively of the atom in question, the tenth column the occupancy of the atom, the eleventh the temperature factor of the atom, the twelfth the chain identifier.

Each table further includes a number of water molecules, designated “TIP”, a N-acteyl D-glucosamine group which is attached to via an N-link to asparagine 146, and designated “NAG 146x” where x is a letter corresponding to the protein chain identifier to which it is linked, and a calcium ion associated with each chain.

Tables 1 to 3 are set out in an internally consistent format. For example (apart from the first residue of Tables 1 and 2), the coordinates of the atoms of each amino acid residue are listed such that the backbone nitrogen atom is first, followed by the C-alpha backbone carbon atom, designated CA, followed by side chain residues (designated according to one standard convention) and finally the carbon and oxygen of the protein backbone. Alternative file formats (e.g. such as a format consistent with that of the EBI Macromolecular Structure Database (Hinxton, UK)) which may include a different ordering of these atoms, or a different designation of the side-chain residues, may be used or preferred by others of skill in the art. However it will be apparent that the use of a different file format to present or manipulate the coordinates of the Table is within the scope of the present invention.

Table 1 comprises eight protein units of the N1 protein, and Table 2 comprises two N4 protein units. In the embodiments of the invention described herein which use the crystal structures of the invention, it will be understood that reference to a “N1 group neuraminidase structure” should be interpreted as the structure of any one individual protein chain. The use of two (or in the case of N1) more units is not excluded, but is not required to practice the present invention. Likewise, reference to a “N1 group neuraminidase structure” does not include solvent, sugar or calcium ion coordinates, though the use of these is not excluded where these may be beneficial or necessary to a particular application of the invention.

Protein structure similarity is routinely expressed and measured by the root mean square deviation (r.m.s.d.), which measures the difference in positioning in space between two sets of atoms. The r.m.s.d. measures distance between equivalent atoms after their optimal superposition. The r.m.s.d. can be calculated over all atoms, over residue backbone atoms (i.e. the nitrogen-carbon-carbon backbone atoms of the protein amino acid residues), main chain atoms only (i.e. the nitrogen-carbon-oxygen-carbon backbone atoms of the protein amino acid residues), side chain atoms only or more usually over C-alpha atoms only. For the purposes of this invention, the r.m.s.d. can be calculated over any of these, using any of the methods outlined below.

Preferably, rmsd is calculated by reference to the C-alpha atoms, provided that where selected coordinates are used, these comprise at least about 5%, preferably at least about 10%, of such atoms. Where selected coordinates do not include said at least about 5%, rmsd may be calculated by reference to all four backbone atoms, provided these comprise at least about 10%, preferably at least about 20% and more preferably at least about 30% of the selected coordinates. Where selected coordinates comprise 90% or more side chain atoms, rmsd may be calculated by reference to all the selected coordinates.

Thus the coordinates of Tables 1 to 3 provide a measure of atomic location in Angstroms, given to 3 decimal places. The coordinates are a relative set of positions that define a shape in three dimensions, but the skilled person would understand that an entirely different set of coordinates having a different origin and/or axes could define a similar or identical shape. Furthermore, the skilled person would understand that varying the relative atomic positions of the atoms of the structure so that the root mean square deviation of the residue backbone atoms (i.e. the nitrogen-carbon-carbon backbone atoms of the protein amino acid residues) is less than 0.75 Å, preferably less than 0.6 Å, more preferably less than 0.5 Å, more preferably less than 0.3 Å, such as less than 0.25 Å, or less than 0.2 Å, and most preferably less than 0.1 Å, when superimposed on the coordinates provided in Tables 1 to 3 for the residue backbone atoms, will generally result in a structure which is substantially the same as the structure of Tables 1 to 3 in terms of both its structural characteristics and usefulness for structure-based analysis of a N1 group neuraminidase protein and its interactivity with molecular structures.

Likewise the skilled person would understand that changing the number and/or positions of the water molecules of the Tables will not generally affect the usefulness of the structures for structure-based analysis of a N1 group neuraminidase protein-interacting structure. Thus for the purposes described herein as being aspects of the present invention, it is within the scope of the invention if: the coordinates of any of Tables 1 to 3 are transposed to a different origin and/or axes; the relative atomic positions of the atoms of the structure are varied so that the root mean square deviation of residue backbone atoms is less than 0.75 Å, preferably less than 0.6 Å, more preferably less than 0.5 Å, more preferably less than 0.3 Å, such as less than 0.25 Å, or less than 0.2 Å, and most preferably less than 0.1 Å when superimposed on the coordinates provided in any of Tables 1 to 3 for the residue backbone atoms; and/or the number and/or positions of water molecules is varied.

In the case of Tables 1 and 2, where the crystal form comprises eight copies of N1 and N4 respectively, the rmsd calculation may be performed using just any one copy of this protein.

Reference herein to the coordinate data of or from Tables 1 to 3, its use, and the like thus includes the coordinate data in which one or more individual values of the Table are varied in this way and will be understood to mean as such unless explicitly stated to the contrary.

Programs for determining rmsd include MNYFIT (part of a collection of programs called COMPOSER, Sutcliffe, M. J., Haneef, I., Carney, D. and Blundell, T. L. (1987) Protein Engineering, 1, 377-384), MAPS (Lu, G. An Approach for Multiple Alignment of Protein Structures (1998, in manuscript and on http://bioinfo1.mbfys.lu.se/TOP/maps.html)).

It is usual to consider C-alpha atoms and the rmsd can then be calculated using programs such as LSQKAB (Collaborative Computational Project 4. The CCP4 Suite: Programs for Protein Crystallography, Acta Crystallographica, D50, (1994), 760-763), QUANTA (Jones et al., Acta Crystallography A47 (1991), 110-119 and commercially available from Accelerys, San Diego, Calif.), Insight (commercially available from Accelerys, San Diego, Calif.), Sybyl®) (commercially available from Tripos, Inc., St Louis), O (Jones et al., Acta Crystallographica, A47, (1991), 110-119), and other coordinate fitting programs.

In, for example the programs LSQKAB and O, the user can define the residues in the two proteins that are to be paired for the purpose of the calculation. Alternatively, the pairing of residues can be determined by generating a sequence alignment of the two proteins, programs for sequence alignment are discussed in more detail herein below. The atomic coordinates can then be superimposed according to this alignment and an r.m.s.d. value calculated. The program Sequoia (C. M. Bruns, I. Hubatsch, M. Ridderström, B. Mannervik, and J. A. Tainer (1999) Human Glutathione Transferase A4-4 Crystal Structures and Mutagenesis Reveal the Basis of High Catalytic Efficiency with Toxic Lipid Peroxidation Products, Journal of Molecular Biology 288(3): 427-439) performs the alignment of homologous protein sequences, and the superposition of homologous protein atomic coordinates. Once aligned, the r.m.s.d. can be calculated using programs detailed above. For sequence identical, or highly identical, the structural alignment of proteins can be done manually or automatically as outlined above. Another approach would be to generate a superposition of protein atomic coordinates without considering the sequence.

It is more normal when comparing significantly different sets of coordinates to calculate the rmsd value over C-alpha atoms only. It is particularly useful when analysing side chain movement to calculate the rmsd over all atoms and this can be done using LSQKAB and other programs.

Thus, for example, varying the atomic positions of the atoms of the structure of Table 1 by up to about 0.5 Å, preferably up to about 0.3 Å in any direction will result in a structure which is substantially the same as the structure of Table 1 in terms of both its structural characteristics and utility e.g. for molecular structure-based analysis. The same applies to Tables 2 and 3.

Those of skill in the art will appreciate that in many applications of the invention, it is not necessary to utilise all the coordinates of Tables 1 to 3, but merely a portion of them. For example, as described below, in methods of modelling molecular structures with a N1 group neuraminidase protein, selected coordinates as referred to herein may be used.

By “selected coordinates” it is meant for example at least 5, preferably at least 10, more preferably at least 50 and even more preferably at least 100, for example at least 500 or at least 1000 atoms of a N1 group neuraminidase protein structure. Likewise, the other applications of the invention described herein, including homology modelling and structure solution, and data storage and computer assisted manipulation of the coordinates, may also utilise all or a portion of the coordinates (i.e. selected coordinates) of Tables 1 to 3. The selected coordinates may include or may consist of atoms found in a binding loop, as described herein below or those which interact with ligands, as described herein below.

Residues of the N1 group neuraminidase proteins involved in ligand binding include Glu-119; Val-149, Asp-151, Arg-156; Arg-224; Tyr-252; His-274; Glu-276; Arg-292; Tyr-347 and Arg-371. In a preferred aspect of the invention, where selected coordinates of a structure of the invention are used, these include one or more atoms of one or more of these residues.

Preferably, the selected coordinates include atoms from at least two, e.g. at least 3, 4, 5, 6, 7, 8 or 9 of the above residues. In one embodiment at least one atom of each of these residues may be used.

Alternatively or in addition, one or more atoms of one or more of the binding loop 149-153 may be used. Thus selected coordinates may include at least one atom from at least two, e.g. at least 3, 4 or all 5 residues of this loop.

In one embodiment, the selected coordinates may include at least one atom from the above-defined loop (with preferred numbers of atoms as defined above) together with at least one atom from a residue selected from Glu-119; Glu-276 and Tyr-347.

### C. Homology Modelling

The invention also provides a means for homology modelling of other proteins (referred to below as target neuraminidase proteins). By “homology modelling”, it is meant the prediction of related neuraminidase protein structure based either on X-ray crystallographic data or computer-assisted de novo prediction of structure, based upon manipulation of the coordinate data derivable from any one of Tables 1 to 3 or selected portions thereof.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

In general, the method involves comparing the amino acid sequences of the a N1 group neuraminidase proteins of any of SEQ ID NOs: 1 to 3 with a target neuraminidase protein by aligning the amino acid sequences. Amino acids in the sequences are then compared and groups of amino acids that are homologous (conveniently referred to as “corresponding regions”) are grouped together. This method detects conserved regions of the polypeptides and accounts for amino acid insertions or deletions.

Homology between amino acid sequences can be determined using commercially available algorithms. The programs BLAST, gapped BLAST, BLASTN, PSI-BLAST and BLAST 2 (provided by the National Center for Biotechnology Information) are widely used in the art for this purpose, and can align homologous regions of two amino acid sequences. These may be used with default parameters to determine the degree of homology between the amino acid sequences of the SEQ ID NOs:1-3 proteins and other target neuraminidase proteins which are to be modelled.

Target proteins include other members of the N1 group of proteins, e.g. N5 as well as mutants or alleles of the N1, N4 or N8 proteins. In the case of the latter, such methods may be used to track changes to neuraminidase proteins which occur in isolates of influenza virus associated with increase pathogenicity or changes to the host species which the virus infects.

Once the amino acid sequences of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization.

Homology modelling as such is a technique that is well known to those skilled in the art (see e.g. Greer, Science, Vol. 228, (1985), 1055, and Blundell et al., Eur. J. Biochem, Vol. 172, (1988), 513). The techniques described in these references, as well as other homology modelling techniques, generally available in the art, may be used in performing the present invention.

Thus the invention provides a method of homology modelling comprising the steps of:
(a) aligning a representation of an amino acid sequence of a target neuraminidase protein of unknown three-dimensional structure with the amino acid sequence of the neuraminidase protein of any one of SEQ ID NOs: 1-3 to match homologous regions of the amino acid sequences;
(b) modelling the structure of the matched homologous regions of said target protein of unknown structure on the corresponding regions of the neuraminidase protein structure from any one of Tables 1 to 3 or selected coordinates thereof; and
(c) determining a conformation (e.g. so that favourable interactions are formed within the target protein of unknown structure and/or so that a low energy conformation is formed) for said target protein of unknown structure which substantially preserves the structure of said matched homologous regions.

Preferably one or all of steps (a) to (c) are performed by computer modelling.

The aspects of the invention described herein which utilise a N1 group neuraminidase protein structure in silico may be equally applied to homologue models obtained by the above aspect of the invention, and this application forms a further aspect of the present invention. Thus having determined a conformation of a target neuraminidase protein by the method described above, such a conformation may be used in computer-based methods, e.g. of rational drug design, as described herein.

### D. Structure Solution

The atomic coordinate data of a N1 group neuraminidase can also be used to solve the crystal structure of other target neuraminidase proteins including other crystal forms of a N1 group neuraminidase protein, co-complexes of a N1 group neuraminidase protein, where X-ray diffraction data or NMR spectroscopic data of these target N1 group neuraminidase proteins has been generated and requires interpretation in order to provide a structure.

For example, a N1 group neuraminidase protein may crystallize in more than one crystal form. The data of Tables 1 to 3, or selected coordinates thereof, as provided by this invention, are particularly useful to solve the structure of those other crystal forms. The data may also be used to solve the structure of a N1 group neuraminidase protein co-complex.

Thus, where X-ray crystallographic or NMR spectroscopic data is provided for a target N1 group neuraminidase protein of unknown three-dimensional structure, the atomic coordinate data derived from any one of Tables 1 to 3 may be used to interpret that data to provide a likely structure for the target by techniques which are well known in the art, e.g. phasing in the case of X-ray crystallography and assisting peak assignments in NMR spectra.

One method that may be employed for these purposes is molecular replacement. In this method, the unknown crystal structure, whether it is another crystal form of a N1 group neuraminidase protein or a co-complex thereof, may be determined using the structure coordinates of all or part of any one of Tables 1 to 3 of this invention. This method will provide an accurate structural form for the unknown crystal more quickly and efficiently than attempting to determine such information ab initio.

Examples of computer programs known in the art for performing molecular replacement are CNX (Brunger A. T.; Adams P. D.; Rice L. M., Current Opinion in Structural Biology, Volume 8, Issue 5, October 1998, Pages 606-611 (also commercially available from Accelrys San Diego, Calif.), MOLREP (A. Vagin, A. Teplyakov, MOLREP: an automated program for molecular replacement, J. Appl. Cryst. (1997) 30, 1022-1025, part of the CCP4 suite) or AMoRe (Navaza, J. (1994). AMoRe: an automated package for molecular replacement. Acta Cryst. A50, 157-163).

Thus, in a further aspect of the invention provides a method for determining the structure of a protein, which method comprises;
providing the coordinates (or selected coordinates thereof) of a N1 group neuraminidase protein structure of any one of Tables 1 to 3,
positioning the coordinates in the crystal unit cell of said protein so as to provide a structure for said protein.

The invention may also be used to assign peaks of NMR spectra of such proteins, by manipulation of the data of any one of Tables 1 to 3.

### E. Computer Systems

In another aspect, the present invention provides systems, particularly a computer system, the systems containing one of (a) co-ordinate data of any one of Tables 1 to 3, said data defining the three-dimensional structure of a N1 group neuraminidase protein or at least selected coordinates thereof; (b) atomic coordinate data of a target neuraminidase protein generated by homology modelling of the target based on the coordinate data from any one of Tables 1 to 3, or (c) atomic coordinate data of a target N1 group neuraminidase protein generated by interpreting X-ray crystallographic data or NMR data by reference to the co-ordinate data from any one of Tables 1 to 3.

For example the computer system may comprise: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational drug design. The computer system may further comprise a display coupled to said central-processing unit for displaying said structures.

The invention also provides such systems containing atomic coordinate data of target proteins as referred to above wherein such data has been generated according to the methods of the invention described herein based on the starting data provided the data of any one of Tables 1 to 3 or selected coordinates thereof.

Such data is useful for a number of purposes, including the generation of structures to analyse the mechanisms of action of neuraminidase proteins and/or to perform rational drug design of compounds, which interact with a N1 group neuraminidase protein, such as compounds which are inhibitors or potential inhibitors of a N1 group neuraminidase protein.

In a further aspect, the present invention provides computer readable media with at least one of (a) co-ordinate data of any one of Tables 1 to 3, said data defining the three-dimensional structure of a N1 group neuraminidase protein or at least selected coordinates thereof; (b) atomic coordinate data of a target neuraminidase protein generated by homology modelling of the target based on the coordinate data from any one of Tables 1 to 3, or (c) atomic coordinate data of a target N1 group neuraminidase protein generated by interpreting X-ray crystallographic data or NMR data by reference to the co-ordinate data from any one of Tables 1 to 3.

As used herein, “computer readable media” refers to any medium or media, which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such computer readable media, the atomic coordinate data of the invention can be routinely accessed to model a N1 group neuraminidase protein or selected coordinates thereof. For example, RASMOL (Sayle et al., TIBS, Vol. 20, (1995), 374) is a publicly available computer software package, which allows access and analysis of atomic coordinate data for structure determination and/or rational drug design.

As used herein, “a computer system” refers to the hardware means, software means and data storage means used to analyse the atomic coordinate data of the invention. The minimum hardware means of the computer-based systems of the present invention comprises a central processing unit (CPU), input means, output means and data storage means. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

A further aspect of the invention provides a method of providing data for generating structures and/or performing optimisation of compounds which interact with a N1 group neuraminidase protein, the method comprising:
(i) establishing communication with a remote device containing
   (a) computer-readable data comprising a N1 group neuraminidase structure or selected coordinates thereof from any one of Tables 1 to 3, optionally varied within a root mean square deviation from the Cα atoms of not more than 0.75 Å; and
(ii) receiving said computer-readable data from said remote device.

Thus the remote device may comprise e.g. a computer system or computer readable media of one of the previous aspects of the invention. The device may be in a different country or jurisdiction from where the computer-readable data is received.

The communication may be via the internet, intranet, e-mail etc, transmitted through wires or by wireless means such as by terrestrial radio or by satellite. Typically the communication will be electronic in nature, but some or all of the communication pathway may be optical, for example, over optical fibers.

Once the data is received from the device, the invention may comprise the further step of using the data in the modelling systems of the invention described herein.

### F. Uses of the Structures of the Invention

The crystal structures obtained according to the present invention as well as the structures of target neuraminidase proteins obtained in accordance with the methods described herein), may be used in several ways for drug design. For example, in order to more fully understand the mechanisms by which influenza virus evolves resistance to current anti-viral drugs, the structures of the present invention may be used to analyse the interactions of anti-viral drugs with mutant enzymes and the structure of the drugs modified to target changes to the N1 group protein.

Information on the binding of such drugs or potential drugs may be obtained by co-crystallization, soaking or computationally docking the drug in the binding pocket. This will guide specific modifications to the chemical structure designed to mediate or control the interaction of the drug with the protein. Such modifications can be designed to improve its therapeutic and/or prophylactic action.

The crystal structures of Group-1 NAs described here, with and without bound inhibitors, reveal that the 150-loop, that forms one corner of the enzyme active site, is able to exist in at least two stable conformations. The fact that Group-1 NAs bind drugs like oseltamivir with similar affinity to Group-2 enzymes suggests that the difference in energy between the two conformations is not very large. This notion of a degree of plasticity in the structure of the active site of influenza virus neuraminidases, or at least of the Group-2 enzymes, is somewhat unexpected. Although we have no direct evidence, from a sequence viewpoint, it would not be surprising if the 150-loop of Group-2 NAs also possessed a similar degree of flexibility. Evidently the ‘closed’ conformation is energetically preferred in Group-2 NAs, both in the absence and presence of current inhibitors, but a higher energy ‘open’ conformation may well be accessible given an inhibitor that makes an energetically advantageous interaction with it and not with the ‘closed’ form.

For example, on the basis of our structural observations we propose that new drugs may be designed by adding extra substitutent moieties to existing inhibitor skeletons. In the first instance designing and refining these molecules against Group-1 NAs may be undertaken as discussed further herein below. Although it might seem ideal to generate influenza drugs that work against all virus subtypes, it is not obvious to us that an effective Group-1 specific inhibitor would not be of considerable value now or in the future. In any case, it may well be possible that new inhibitors designed to exploit additional interactions with the open form of the 150-loop of Group-1 NAs could select a similar conformation in this loop in Group-2 NAs.

What is still likely to be the case is that clinical use of any new drugs will eventually lead to the appearance of resistant mutations. Lessons from treating retroviral diseases suggest that one strategy to overcome this problem is the use of combination drug therapy.

### (i) Obtaining and Analysing Crystal Complexes.

In one approach, the structure of a compound bound to a N1 group neuraminidase protein may be determined by experiment. This will provide a starting point in the analysis of the compound bound to a N1 group neuraminidase protein, thus providing those of skill in the art with a detailed insight as to how that particular compound interacts with a N1 group neuraminidase protein and the mechanism by which it works.

Many of the techniques and approaches to structure-based drug design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map (as explained e.g. by Blundell et al., in Protein Crystallography, Academic Press, New York, London and San Francisco, (1976)), it is necessary to know beforehand the protein 3D structure (or at least the protein structure factors). Therefore, determination of the N1 group neuraminidase protein structures also allows difference Fourier electron density maps of protein-compound complexes to be produced, determination of the binding position of the drug and hence may greatly assist the process of rational drug design.

Accordingly, the invention provides a method for determining the structure of a compound bound to a N1 group neuraminidase protein, said method comprising:
providing a crystal of a N1 group neuraminidase protein according to the invention;
soaking the crystal with said compounds; and
determining the structure of said N1 group neuraminidase protein compound complex by employing the coordinate data of any one of Tables 1 to 3 or selected coordinates thereof.

Alternatively, the N1 group neuraminidase protein and compound may be co-crystallized. Thus the invention provides a method for determining the structure of a compound bound to a N1 group neuraminidase protein said method comprising;
mixing the protein with the compound(s), crystallizing the protein-compound(s) complex; and determining the structure of said protein-compound(s) complex by reference to the coordinate data of any one of Tables 1 to 3 or selected coordinates thereof.

The analysis of such structures may employ (i) X-ray crystallographic diffraction data from the complex and (ii) a three-dimensional structure of a N1 group neuraminidase protein, or at least selected coordinates thereof, to generate a difference Fourier electron density map of the complex, the three-dimensional structure being defined by atomic coordinate data of any one of Tables 1 to 3 or selected coordinates thereof. The difference Fourier electron density map may then be analysed.

Therefore, such complexes can be crystallized and analysed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked or co-crystallized protein and the solved structure of uncomplexed protein. These maps can then be analysed e.g. to determine whether and where a particular compound binds to a N1 group neuraminidase protein and/or changes the conformation of said protein.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualization and model building programs such as “O” (Jones et al., Acta Crystallographica, A47, (1991), 110-119) can be used.

All of the complexes referred to above may be studied using well-known X-ray diffraction techniques and may be refined against 1.5 to 3.5 Å resolution X-ray data to an R value of about 0.30 or less using computer software, such as CNX (Brunger et al., Current Opinion in Structural Biology, Vol. 8, Issue 5, October 1998, 606-611, and commercially available from Accelrys, San Diego, Calif.), and as described by Blundell et al, (1976) and Methods in Enzymology, vol. 114 & 115, H. W. Wyckoff et al., eds., Academic Press (1985).

(ii) In silico analysis and design

Although the invention will facilitate the determination of actual crystal structures comprising a N1 group neuraminidase protein and a compound, which interacts with the protein, current computational techniques provide a powerful alternative to the need to generate such crystals and generate and analyse diffraction date. Accordingly, a particularly preferred aspect of the invention relates to in silico methods directed to the analysis and development of compounds which interact with N1 group neuraminidase protein structures of the present invention.

Determination of the three-dimensional structure of a N1 group neuraminidase protein provides important information about the binding sites of this protein, particularly when comparisons are made with similar proteins. As set out in the accompanying examples, we have identified surprising and significant differences in the binding pocket of the N1 proteins in the 149-150 loop, resulting in a significant displacement of some of the residues in this pocket compared to the N2 group of neuraminidase proteins. Additionally, the change of position 347 to tyrosine in the N1 group results in a further ligand interaction not found in the N2 group. Thus in the design of the next generation of neuraminidase inhibitors, attention can be focussed on these newly-identified differences to provide for improved compounds which may overcome the resistance observed against currently available drugs.

This information may then be used for rational design and modification of neuraminidase inhibitors, e.g. by computational techniques which identify possible binding ligands for the binding sites, by enabling linked-fragment approaches to drug design, and by enabling the identification and location of bound ligands (e.g. including those ligands mentioned herein above) using X-ray crystallographic analysis. These techniques are discussed in more detail below.

Thus as a result of the determination of the three-dimensional structures of N1 group neuraminidase proteins, more purely computational techniques for rational drug design may also be used to design structures whose interaction with these group of proteins is better understood (for an overview of these techniques see e.g. Walters et al (Drug Discovery Today, Vol. 3, No. 4, (1998), 160-178; Abagyan, R.; Totrov, M. Curr. Opin. Chem. Biol. 2001, 5, 375-382). For example, automated ligand-receptor docking programs (discussed e.g. by Jones et al. in Current Opinion in Biotechnology, Vol. 6, (1995), 652-656 and Halperin, I.; Ma, B.; Wolfson, H.; Nussinov, R. Proteins 2002, 47, 409-443), which require accurate information on the atomic coordinates of target receptors may be used.

The aspects of the invention described herein which utilize the N1 group neuraminidase protein structure in silico may be equally applied to both the structure from any one of Tables 1 to 3 or selected coordinates thereof and the models of target neuraminidase proteins obtained by other aspects of the invention. Thus having determined a conformation of neuraminidase protein by the method described above, such a conformation may be used in a computer-based method of rational drug design as described herein. In addition the availability of the structure of the a N1 group neuraminidase protein will allow the generation of highly predictive pharmacophore models for virtual library screening or compound design.

Accordingly, the invention provides a computer-based method for the analysis of the interaction of a molecular structure with a N1 group neuraminidase structure, which comprises:
providing a N1 group neuraminidase structure or selected coordinates thereof from any one of Tables 1 to 3, optionally varied within a root mean square deviation from the Cα atoms of not more than 0.75 Å;
providing a molecular structure to be fitted to said N1 group neuraminidase structure or selected coordinates thereof; and
fitting the molecular structure to said N1 group neuraminidase structure.

In practice, it will be desirable to model a sufficient number of atoms of a N1 group neuraminidase structure as defined by the coordinates from any one of Tables 1 to 3 or selected coordinates thereof), which represent a binding pocket, e.g. the numbers of atoms or the atoms from preferred residues as defined in section B above. Thus in this aspect of the invention, the selected coordinates may comprise coordinates of some or all of these above-mentioned residues.

Following the fitting of the molecular structures, a person of skill in the art may seek to use molecular modelling to determine to what extent the structures interact with each other (e.g. by hydrogen bonding, other non-covalent interactions, or by reaction to provide a covalent bond between parts of the structures).

The person of skill in the art may use in silico modelling methods to alter one or more of the structures in order to design new structures which interact in different ways with a N1 group neuraminidase structure.

Newly designed structures may be synthesised and their interaction with a N1 group neuraminidase structure may be determined or predicted as to how the newly designed structure is bound by said N1 group neuraminidase structure. This process may be iterated so as to further alter the interaction between it and the a N1 group neuraminidase structure.

By “fitting”, it is meant determining by automatic, or semi-automatic means, at least one interaction between at least one atom of a molecular structure and at least one atom of a N1 group neuraminidase structure of the invention, and calculating the extent to which such an interaction is stable. Interactions include attraction and repulsion, brought about by charge, steric considerations and the like. Various computer-based methods for fitting are described further herein.

More specifically, the interaction of a compound or compounds with a N1 group neuraminidase structure can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al, J. Mol. Biol. 1982, 161, 269-288, Makino et al, J. Comput. Chem. 1997, 18, 1812-1825), AUTODOCK (Goodsell et al, Proteins 1990, 8, 195-202, Morris et al, J. Comput. Chem. 1998, 19, 1639-1662.), FlexX, (Rarey et al, J. Mol. Biol. 1996, 261, 470-489) or ICM (Abagyan et al, J. Comput. Chem. 1994, 15, 488-506). This procedure can include computer fitting of compounds to a N1 group neuraminidase structure to ascertain how well the shape and the chemical structure of the compound will bind to the structure.

Also computer-assisted, manual examination of the active site structure of a N1 group neuraminidase may be performed. The use of programs such as GRID (Goodford, J. Med. Chem., 28, (1985), 849-857)—a program that determines probable interaction sites between molecules with various functional groups and an enzyme surface—may also be used to analyse the active site to predict, for example, the types of modifications which will alter the rate of metabolism of a compound.

Computer programs can be employed to estimate the attraction, repulsion, and steric hindrance of the two binding partners.

Detailed structural information can then be obtained about the binding of the compound to a N1 group neuraminidase structure, and in the light of this information adjustments can be made to the structure or functionality of the compound, e.g. to alter its interaction with a N1 group neuraminidase structure. The above steps may be repeated and re-repeated as necessary.

Molecular structures, which may be used in the present invention, will usually be compounds under development for pharmaceutical use. Generally such compounds will be organic molecules, which are typically from about 100 to 2000 Da, more preferably from about 100 to 1000 Da in molecular weight. Such compounds include peptides and derivatives thereof, and sialic-acid derivatives including derivatives of oseltamivir, zanamivir, DANA and peramivir. In principle, any compound under development in the field of pharmacy can be used in the present invention in order to facilitate its development or to allow further rational drug design to improve its properties.

In another embodiment, the present invention provides a method for modifying the structure of a compound in order to alter its interaction with a N1 group neuraminidase, which method comprises:
fitting a starting compound to one or more coordinates of at least one amino acid residue of the ligand-binding region of a N1 group neuraminidase structure of the present invention;
modifying the starting compound structure so as to increase or decrease its interaction with the ligand-binding region;
wherein said ligand-binding region is defined as including at least one, and preferably more than one, of the residues of Glu-119; Val-149, Asp-151, Arg-156; Arg-224; Tyr-252; His-274; Glu-276; Arg-292; Tyr-347 and Arg-371 and/or the amino acids 149-152. Preferred numbers and combinations of residues are as defined herein above.

For the avoidance of doubt, the term “modifying” is used as defined in the preceding subsection, and once such a compound has been developed it may be synthesised and tested also as described above.

(iii) Fragment Linking and Growing.

The provision of the crystal structures of the invention will also allow the development of compounds which interact with the binding pocket regions of a N1 group neuraminidase (for example to act as an inhibitors of the protein) based on a fragment linking or fragment growing approach.

For example, the binding of one or more molecular fragments can be determined in the protein binding pocket by X-ray crystallography. Molecular fragments are typically compounds with a molecular weight between 100 and 200 Da (Carr et al, 2002). This can then provide a starting point for medicinal chemistry to optimise the interactions using a structure-based approach. The fragments can be combined onto a template or used as the starting point for ‘growing out’ an inhibitor into other pockets of the protein (Blundell et al, 2002). The fragments can be positioned in the binding pocket of a N1 group neuraminidase structure and then ‘grown’ to fill the space available, exploring the electrostatic, van der Waals or hydrogen-bonding interactions that are involved in molecular recognition. The potency of the original weakly binding fragment thus can be rapidly improved using iterative structure-based chemical synthesis.

At one or more stages in the fragment growing approach, the compound may be synthesized and tested in a biological system for its activity. This can be used to guide the further growing out of the fragment.

Where two fragment-binding regions are identified, a linked fragment approach may be based upon attempting to link the two fragments directly, or growing one or both fragments in the manner described above in order to obtain a larger, linked structure, which may have the desired properties.

Where the binding site of two or more ligands are determined they may be connected to form a potential lead compound that can be further refined using e.g. the iterative technique of Greer et al. For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design neuraminidase inhibitors is made possible by the determination of the neuraminidase structure.

### (iv) Compounds of the Invention.

Where a potential modified compound has been developed by fitting a starting compound to a N1 group neuraminidase structure of the invention and predicting from this a modified compound with an altered rate of action (including a slower, faster or zero rate), the invention further includes the step of synthesizing the modified compound and testing it in an in vivo or in vitro biological system in order to determine its activity and/or the rate at which it acts, e.g. to inhibit viral growth or spread.

In another aspect, the invention includes a compound, which is identified by the methods of the invention described above.

Following identification of such a compound, it may be manufactured and/or used in the preparation, i.e. manufacture or formulation, of a composition such as a medicament, pharmaceutical composition or drug. These may be administered to individuals.

Thus, the present invention extends in various aspects not only to a compound as provided by the invention, but also a pharmaceutical composition, medicament, drug or other composition comprising such a compound. The compositions may be used. for treatment (which may include preventative treatment) of disease, particularly influenza A or B. Such a treatment may comprise administration of such a composition to a patient, e.g. for treatment of disease; the use of such an inhibitor in the manufacture of a composition for administration, e.g. for treatment of disease; and a method of making a pharmaceutical composition comprising admixing such an inhibitor with a pharmaceutically acceptable excipient, vehicle or carrier, and optionally other ingredients.

Thus a further aspect of the present invention provides a method for preparing a medicament, pharmaceutical composition or drug, the method comprising (a) identifying or modifying a compound by a method of any one of the other aspects of the invention disclosed herein; (b) optimising the structure of the molecule; and (c) preparing a medicament, pharmaceutical composition or drug containing the optimised compound.

The above-described processes of the invention may be iterated in that the modified compound may itself be the basis for further compound design.

By “optimising the structure” we mean e.g. adding molecular scaffolding, adding or varying functional groups, or connecting the molecule with other molecules (e.g. using a fragment linking approach) such that the chemical structure of the modulator molecule is changed while its original modulating functionality is maintained or enhanced. Such optimisation is regularly undertaken during drug development programmes to e.g. enhance potency, promote pharmacological acceptability, increase chemical stability etc. of lead compounds.

Modification will be those conventional in the art known to the skilled medicinal chemist, and will include, for example, substitutions or removal of groups containing residues which interact with the amino acid side chain groups of a N1 group neuraminidase structure of the invention. For example, the replacements may include the addition or removal of groups in order to decrease or increase the charge of a group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The invention is illustrated by the following examples:

### EXAMPLES

### Crystallisation of N1 Neuraminidase

N1 protein of SEQ ID NO:1 was prepared from A/Vietnam/1203/04 virus grown in hens' eggs. NA was released from the virus by bromelain digestion and further purified as described previously (Ha Y, Stevens D J, Skehel J J & Wiley D C (2001) Proc Natl Acad Sci Sep. 25; 98(20) 11181-6) to produce a crystallisable protein of residues 62449 of SEQ ID NO:1.

The protein crystallizes under three conditions: (1) 20% PEG 3350, 0.2 M ammonium acetate, 0.1M MES pH 6.0; (2) 20% PEG 3350, 0.2 M ammonium acetate, 0.1M PIPES pH 6.8; (3) 20% PEG 3350, 0.2 M lithium sulphate, 0.1M TrisCl pH 8.5.

Accordingly, the invention in one aspect provides a method of crystallizing a N1 protein of the invention under any one of conditions (1)-(3), wherein each reagent may be varied independently by up to 5% in concentration, pH, or, in the case of PEG, molecular weight.

Crystals from condition 1 diffracted and were these conditions were used for subsequent optimisation on a larger scale. Hanging drops were set up using 1 μl N1 protein (9A₂₈₀/ml) plus 1 μl well solution equilibrated against 18-23% PEG 3350, 0.2M ammonium acetate, 0.1M MES pH 6.0. Crystals from drops containing 20 or 21% PEG were used for native data collection and soaks with 20 μM or 0.5 mM Tamiflu® (oseltamavir).

The cryoprotectant solution used for the crystals was 21% PEG 3350, 0.2M ammonium acetate, 0.1M MES pH 6.0, 15% ethylene glycol.

### Crystallization of N4 and N8 Neuraminidase.

### Methods

N4 (SEQ ID NO:2) and N8 (SEQ ID NO:3) NAs were prepared from A/Mink/Sweden/E12665/84 (H10N4) and A/Duck/Ukraine/1/63 (H3N8) viruses grown in hens' eggs. NA was released from the viruses by bromelain digestion to provide N4 79-470 and N8 73-470, and further purified, as previously described above. Protein was recovered at a concentration of 10 mg/ml in 10 mM Tris-HCl, pH 8.0.

N4 NA Crystals were grown by vapour diffusion in hanging drops consisting of 2 μl of reservoir solution (0.1M Hepes, pH 7.5, 5 mM cobalt chloride, 5 mM nickel chloride, 5 mM cadmium chloride, 5 mM magnesium chloride and 12% w/v PEG 3350) and 2 μl of concentrated protein solution.

Accordingly, the invention in one aspect provides a method of crystallizing a N4 protein of the invention under the above conditions, wherein each reagent may be varied independently by up to 5% in concentration, pH, or, in the case of PEG, molecular weight.

N8 NA Crystals were grown by vapour diffusion in hanging drops consisting of 2 μl of reservoir solution (0.1M Imidazole, pH 8.0 and 35% MPD) and 2 μl of concentrated protein solution (10 mg/ml in 10 mM Tris-HCl, pH 8.0).

Accordingly, the invention in one aspect provides a method of crystallizing a N8 protein of the invention under the above conditions, wherein each reagent may be varied independently by up to 5% in concentration, pH, or, in the case of PEG, molecular weight.

Crystals were soaked for 30 minutes in 20 mM inhibitor made up in crystallisation buffer (augmented with 20% glycerol, in the case of N4, for cryoprotection). Additionally a crystal of N8 NA was soaked in 20 mM oseltamivir for 3 days. Data were collected at 100K on an in-house Rigaku-MSC RU200 rotating anode coupled to a Raxisllc detector. Diffraction data were integrated using Denzo and scaled with Scalepack. N4 and N8 NA structures were solved by molecular replacement using Phaser with N9 NA as the search model. Standard refinement, with CNS and manual model building, with O, was performed. Crystallographic statistics are given in Table 4. All figures were created with Pymol.

The crystal structures of N1, N4 and N8 were solved by molecular replacement and relevant crystallographic statistics are shown in Table 4. Table 5 shows the form, dimensions and resolution of the crystals obtained. The structures of unliganded N1, N4 and N8 are set out in Tables 1 to 3 respectively.

TABLE 4

| | Rwork | Rfree | B-factor | B-factor |
|---|---|---|---|---|
| NA subtype | (%) | (%) | protein (Å²) | ligand (Å²) |
| N1 uncomplexed | 23.2 | 26.2 | 26.3 | — |
| +oseltamivir | 23.7 | 27.3 | 21.4 | 25.1 |
| N4 uncomplexed | 22.2 | 27.3 | 24.2 | — |
| +DANA | 21.7 | 29.5 | 16.0 | 20.0 |
| N8 uncomplexed | 21.7 | 26.1 | 31.6 | — |
| +oseltamivir (30 mins) | 22.9 | 28.6 | 22.8 | 33.3 |
| +oseltamivir (3 days) | 25.2 | 27.3 | 29.8 | 31.6 |
| +DANA | 21.7 | 26.7 | 15.6 | 16.8 |
| +Zanamivir | 27.1 | 33.4 | 27.1 | 27.2 |
| +Peramivir | 23.4 | 28.6 | 19.0 | 15.6 |

Table 4: Crystallographic statistics.

R_{work}=Σ∥Fo|−|Fc∥/Σ|Fo|.

R_{free}=Σ_{T}∥Fo|−|Fc∥/Σ_{T}|Fo|, where T is a test data set of 5% of the total reflections randomly chosen and set aside before refinement.

TABLE 5

| | Space | Unit cell | | Resolution |
|---|---|---|---|---|
| Crystal | group | dimensions (Å) | Z | (Å) |
| N1 | C222₁ | a = 201.74, | 8 | 2.5 |
| | | b = 201.51, | | |
| | | c = 212.43 | | |
| N1 + oseltamivir | C222₁ | a = 200.62, | 8 | 2.5 |
| | | b = 200.70, | | |
| | | c = 210.48 | | |
| N4 | P432 | a = 193.79 | 2 | 2.8 |
| N4 + DANA | I432 | a = 193.44 | 1 | 3.5 |
| N8 | I4 | a = b = 90.67, | 1 | 2.4 |
| | | c = 109.4 | | |
| N8 + DANA | I4 | a = b = 90.38, | 1 | 2.5 |
| | | c = 111.49 | | |
| N8 + Oseltamivir | I4 | a = b = 90.58, | 1 | 2.6 |
| (30 mins soak) | | c = 110.78 | | |
| N8 + Oseltamivir | I4 | a = b = 90.42, | 1 | 2.4 |
| (3 day soak) | | c = 109.71 | | |
| N8 + Zanamivir | I4 | a = b = 90.41, | 1 | 2.2 |
| | | c = 109.30 | | |
| N8 + Peramivir | I4 | a = b = 89.78, | 1 | 2.2 |
| | | c = 93.23 | | |

### Active Site Comparison

Superposition of the structures of N1, N4 and N8 Group-1 NAs reveals that their active sites are virtually identical. However, there are substantial conformational differences between Group-1 and Group-2 centred on the 150-loop (residues 147-152) and the 150-cavity adjacent to the active site. The conformation of the loop is such that the C-alpha position of Group-1 specific Val-149 is about 7 Å distant from the equivalent isoleucine residue in Group-2. Moreover, the hydrophobic side chain at position 149 is pointed away from the active site in Group-1 but towards it in Group-2. At the point of closest approach of the 150-loop to the active site, there is a difference of 1.5 Å in the side chain position of the catalytically important aspartic acid residue at position 151 between Group-1 and Group-2. Comparison of the amino acid residues in the 150-loops offers no obvious explanation for the strong conservation of loop structure within, but not between Group-1 and Group-2.

It is apparent that this loop conformation is an intrinsic feature of Group-1 and not a consequence of adventitious lattice contacts because all three Group-1 enzyme structures of the invention, which crystallise under different conditions, and in different space groups, show a similar structure here.

A major consequence of these differences in structure is that there is a large cavity adjacent to the active site in Group-1 but not in Group-2 NAs. This cavity is accessible from the active site because of the differences in position of Asp-151 and Glu-119 described above. The combined effect of the difference in position of these two acidic residues is to increase the width of the active site cavity by about 5 Å. The conserved Arg-156, whose side chain is located approximately mid-way between the two acidic residues, adopts approximately the same position in the Group-1 and Group-2 structures and defines the entrance from the active site cavity into the 150-cavity. The extent of the 150-cavity is then determined by the difference in conformation of the 150-loop and by the position of Gin-136. In Group-2 proteins this residue hydrogen bonds with the main chain carbonyl of residue 150 of the loop. In Group-1 structures, presumably as a consequence of the different loop structure, Gln-136, unable to make this hydrogen bond, adopts a conformation that results in its side chain sitting about 3.5 Å lower at the base of the cavity. The 150-cavity is therefore about 10 Å long and 5 Å wide and deep. This cavity and its accessibility to the active site may have important implications for the development of drugs which are more specific for the Group-1 proteins.

It would not be apparent from homology modelling or similar techniques based on currently available Group-2 NA structures that such a difference exists.

Two other notable differences between the unliganded structures of Group-1 and Group-2 involve the side chain conformations of Glu-276 and Glu-119. The conformation of Glu-276 is of particular interest because it undergoes the most significant rearrangement upon drug binding to NAs from Influenza B and Group-2. For example, in unliganded Group-2 (N9) the carboxylate of Glu-276 faces into the active site but upon oseltamivir binding it adopts a conformation pointing away from the active site so that the carboxylate now makes a bidentate interaction with the guanidinium group of Arg-224. In so doing the hydrophobic CB and CG of Glu-276 move towards the C6-linked hydrophobic substituent of oseltamivir. In unliganded Group-1 NAs the conformation of Glu-276 is more like the ligand bound conformation seen in Group-2. Thus, although the CD atom of Glu-276 in unliganded N1 is about 1 Å away from the position of the equivalent atom in oseltamivir bound N9, its carboxylate is still able to make the same bidentate interaction with Arg-224. Glu-119 adopts a conformation in Group-1 such that its carboxylate points in approximately the opposite direction as it does in Group-2.

### Inhibitor Binding

We have determined the crystal structures of various anti-neuraminidase inhibitors in complex with N1, N4 and N8 of Group 1 as summarised in Table 4. Remarkably, we find that Group-1 NAs can bind oseltamivir in either the ‘open’ or ‘closed’ conformation of the 150-loop depending on the soaking conditions. Thus the structure of N8 NA in complex with oseltamivir, resulting from a 30-minute soak of inhibitor into preformed crystals, reveals that no large-scale conformational changes have occurred and that the 150-loop retains the same conformation as in the unliganded structure. Presumably as a consequence of the conformation of the 150-loop the acidic residues Asp-151 and Glu-119 are located further from the nitrogen attached to C4 of the inhibitor than they are in the complex with N9. Other interactions between oseltamivir and the N8 NA are similar to those observed in N9 with the further exception that Tyr-347 makes a hydrogen bond interaction with the C1 carboxylate of oseltamivir in addition to the usual bidentate interaction of that carboxylate with Arg-371. In Group-2 residue 347 is a glutamine, rather than a tyrosine, that is unable to make such a hydrogen bond.

The observation that oseltamivir can bind to N8 with the 150-loop in the open conformation seems important. It would be possible to obtain this result if there were low occupancy of the ligand in the crystals. However, the quality of the X-ray data and model refinement is such that we are confident that this is not the case. Rather, it seems likely that the binding of oseltamivir to N8, at least in the crystalline state, is a two-step process. Firstly, inhibitor binds to the ‘open’ form of N8 and then a slow conformational change occurs that results in the ‘closed’ form of the enzyme that realises more of the energy of interaction with ligand. Our structural observations show that this type of inhibitor is capable of binding to the ‘open’ conformation of Group-1 NAs.

When N8 crystals were incubated in oseltamivir for 3 days, or N1 crystals were incubated in a higher concentration of inhibitor, the 150-loop changes its conformation so that it closely resembles the conformation observed in Group-2 in the presence and absence of inhibitors. There are two main consequences of this change in conformation. Firstly, Glu-119 and Asp-151 are now both oriented toward the bound oseltamivir and, secondly, the size of the active site cavity in drug-bound Group-1 is now much the same as it is for Group-2 NAs. We have also determined the structures of three other neuraminidase inhibitors, DANA, zanamivir and peramivir bound to Group-1. Overall, these structures show that the drug bound complexes of Group-1 are very similar to those seen for Group-2. In all three cases the 150-loop of Group-1 changes its conformation on drug binding bringing Asp-151 closer to the inhibitor and, in so doing, closing the 150-cavity.

The discovery of the ‘open’ conformation for the 150-loop in the Group-1 structures suggests that, for these enzymes, this conformation is intrinsically lower in energy than the ‘closed’ conformation for this loop. Group-1 (N8) initially binds to oseltamivir in this ‘open’ conformation but eventually adopts the closed conformation. It thus appears that oseltamivir binding to Group-1 favours the higher energy or ‘closed’ conformer of the 150-loop which it probably accesses via a relatively slow conformational change. It should therefore be possible to design novel inhibitors for Group-1 that are selective for the ‘open’ 150-loop conformation and would thereby have the potential to bind more strongly than Oseltamivir or Zanamivir.

Examination of our structures suggests, for example, that it may be possible for a side chain to be developed from the 4-amino group of Oseltamivir, or the corresponding guanidinium cation in zanamivir, into the 150-cavity and thereby enhance the binding of novel inhibitors to Group-1 relative to Group-2 neuraminidases. The cavity opens near C4 of oseltamivir and zanamivir and contains, at its base, the prominent guanidinium side-chain of conserved Arg-156, that is a prospective partner for an internal salt-bridge or hydrogen-bond pair for a new inhibitor.

### Inhibitor Binding

### Differential Oseltamivir Resistance of Group-1 and Group-2 Mutant NAs

Three oseltamivir and/or zanamivir-resistant mutant NAs have been characterized from influenza A viruses isolated following Tamiflu treatment of influenza-infected humans. One, derived from H5N1 infections, contained the amino acid substitution His-274->Tyr. The other two were from patients infected with H3N2 viruses and contained either Glu-119->Val or Arg-292->Lys substitutions. Comparison of the structures of Group-1 and Group-2 NAs reveals group specific differences in the active sites that might explain how these mutations lead to inhibitor resistance.

### His-274->Tyr

The mutation His-274->Tyr leads to high resistance of Group-1 NAs against oseltamivir but has little effect on Group-2 NAs. Inspection of the structures of the Group-1 NAs in complex with oseltamivir, and comparison with equivalent Group-2 complexes, suggests a reason for this group-specific behaviour and indicates how resistance maybe mediated by the effects of the mutant Tyr-224 on the orientation of Glu-276. The importance of the conformation of Glu-276 for oseltamivir binding by Group-2 NAs has been firmly established. There appears to be at least two factors contributing to the inability of Group-1 NAs to accommodate the His-274Tyr substitution. Firstly, the 270-loop in Group-1 NAs approaching residue 273 makes a tighter turn than the equivalent loop in Group-2. Secondly, in Group-1, but not in Group-2, there is a conserved tyrosine residue at position 252 that makes hydrogen bonds to the main chain carbonyl at position 273, to the peptide amide at 250 and to the histidine side chain at 274. His-274 also hydrogen bonds through its other side chain nitrogen with Glu-276. It appears that introduction of the bulkier tyrosine residue at position 274 in Group-1 enzymes can only be accommodated by the new side chain moving towards, and partially displacing, Glu-276. By contrast, in Group-2 enzymes, there is a smaller residue at position 252 leaving space for tyrosine 274 to occupy without perturbing Glu-276. This interpretation of the group-specific effect of this mutation is consistent with observations from mutagenic studies reported earlier.

### Arg-292->Lys

The mutation Arg-292->Lys is the commonest substitution in Group-2 NAs resistant to oseltamivir. It has already been the subject of a detailed crystallographic analysis to show that in N9 NA resistance results in part from the loss of a hydrogen bond from Arg-292 to the carboxylate group of oseltamivir. The substituted Lys-292 also interacts with Glu-276 impeding its movement to accommodate the hydrophobic substituent attached to C6 of oseltamivir. The structures of Group-1 NAs, and their complexes with oseltamivir, now reveal a likely reason for the smaller effect of the mutation on Group-1 enzymes. The conserved tyrosine residue at position 347 in Group-1 makes an additional hydrogen bond to the carboxylate group of the inhibitor that cannot be made by the equivalent residues in Group-2. In this way it seems that the additional hydrogen bond interaction between Tyr-347 and the carboxylate of the inhibitor compensates for a weaker, water-mediated, interaction between the carboxylate and the substituted lysine residue at position 292.

All publications and patents mentioned in the above specification are herein incorporated by reference. Various modifications and variations of the described invention will be apparent to those of skill in the art without departing from the scope and spirit of the invention. Although the invention has been described in connection with specific preferred embodiments, it should be understood that the invention as claimed should not be unduly limited to such specific embodiments.

1. A computer-based method for the analysis of the interaction of a molecular structure with a N1 group neuraminidase structure, which comprises:
providing a N1 group neuraminidase structure or selected coordinates thereof from any one of Tables 1 to 3, optionally varied within a root mean square deviation from the Cα atoms of not more than 0.75 Å;
providing a molecular structure to be fitted to said N1 group neuraminidase structure or selected coordinates thereof; and
fitting the molecular structure to said N1 group neuraminidase structure.

2. The method of claim 1 wherein said selected coordinates include atoms from one or more of the residues of Glu-119; Val-149, Asp-151, Arg-156; Arg-224; Tyr-252; His-274; Glu-276; Arg-292; Tyr-347 and Arg-371.

3. The method of claim 1 which further comprises the steps of:
obtaining or synthesising a compound which has said molecular structure; and
contacting said compound with an N1 group neuraminidase protein to determine the ability of said compound to interact with the N1 group neuraminidase.

4. The method of claim 1 which further comprises the steps of:
obtaining or synthesising a compound which has said molecular structure;
forming a complex of a N1 group neuraminidase protein and said compound; and
analysing said complex by X-ray crystallography to determine the ability of said compound to interact with the N1 group neuraminidase.

5. The method of claim 1 which further comprises the steps of:
obtaining or synthesising a compound which has said molecular structure; and
determining or predicting how said compound interacts with said N1 group neuraminidase structure; and
modifying the compound structure so as to alter the interaction between it and the N1 group neuraminidase.

6. A compound having the modified structure identified using the method of claim 6.

7. The method of claim 1 wherein the selected coordinates are of at least 5, 10, 50, 100, 500 or 1000 atoms.

8. The method of claim 1 wherein the selected coordinates of any one of Tables 1 to 3 represent at least one side chain atom of each of residues 147-152.

9. The method of claim 8 wherein the selected coordinates further include at least one side-chain residue of an amino acid selected from Glu-119; Glu-276 and Tyr-347.

10. A method for determining the structure of a compound bound to a N1 group neuraminidase protein, said method comprising:
providing a crystal of said N1 group neuraminidase protein;
soaking the crystal with the compound to form a complex; and
determining the structure of the complex by employing the data from any one of Tables 1 to 3, optionally varied within a root mean square deviation from the Cα atoms of not more than 0.75 Å, or selected coordinates thereof.

11. A method for determining the structure of a compound bound to a N1 group neuraminidase protein, said method comprising:
mixing a N1 group neuraminidase protein with the compound;
crystallizing a protein-compound complex; and
determining the structure of the complex by employing the data from any one of Tables 1 to 3, optionally varied within a root mean square deviation from the Cα atoms of not more than 0.75 Å, or selected coordinates thereof.

12. A method of providing data for generating structures and/or performing optimisation of compounds which interact with a N1 group neuraminidase protein, the method comprising:
(i) establishing communication with a remote device containing
(a) computer-readable data comprising a N1 group neuraminidase structure or selected coordinates thereof from any one of Tables 1 to 3, optionally varied within a root mean square deviation from the Cα atoms of not more than 0.75 Å; and
(ii) receiving said computer-readable data from said remote device.

13. The method of claim 12 which further comprises performing the method for the analysis of the interaction of a molecular structure with a N1 group neuraminidase structure, which comprises:
providing a N1 group neuraminidase structure or selected coordinates thereof from any one of Tables 1 to 3, optionally varied within a root mean square deviation from the Cα atoms of not more than 0.75 Å;
providing a molecular structure to be fitted to said N1 group neuraminidase structure or selected coordinates thereof; and
fitting the molecular structure to said N1 group neuraminidase structure with said data.

14. A crystal of a N1 group neuraminidase protein.

15. A co-crystal of a N1 group neuraminidase protein and a ligand.

16. A co-crystal according to claim 15 wherein said ligand is selected from the group oseltamivir, zanamivir, DANA and peramivir, or derivatives thereof.

17. A crystal or co-crystal of claim 14 wherein said N1 group neuraminidase protein is selected from N1, N4 and N8.

18. The crystal or co-crystal of claim 14 wherein said neuraminidase N1 group protein is a N1 protein of residues 62-449 of SEQ ID NO:1 or a variant thereof having from 1 to 10 amino acid substitutions, deletions or insertions.

19. The crystal or co-crystal of claim 14 wherein said neuraminidase N1 group protein is a N1 protein having an C-orthorhombic space group C222₁.

20. The crystal or co-crystal of claim 19 having unit cell dimensions a=200.21 Å, b=200.77 Å, c=211.68 Å, alpha=90 beta=90 gamma=90, with a unit cell variability of 5% in all dimensions.

21. The crystal or co-crystal of claim 14 wherein said neuraminidase N1 group protein is a N4 protein of residues 79-470 of SEQ ID NO:2 or a variant thereof having from 1 to 10 amino acid substitutions, deletions or insertions.

22. The crystal or co-crystal of claim 14 wherein said neuraminidase N1 group protein is a N4 protein having an cubic space group P432 or I432.

23. The crystal or co-crystal of claim 22 having unit cell dimensions a=b=c=193.79 Å, with a unit cell variability of 5% in all dimensions.

24. The crystal or co-crystal of claim 14 wherein said neuraminidase N1 group protein is a N8 protein of residues 73-470 of SEQ ID NO:3 or a variant thereof having from 1 to 10 amino acid substitutions, deletions or insertions.

25. The crystal or co-crystal of claim 14 wherein said neuraminidase N1 group protein is a N8 protein having an tetragonal space group I4.

26. The crystal or co-crystal of claim 25 having unit cell dimensions having unit cell dimensions a=b=90.67 Å, c=c=109.4 Å, alpha=90 beta=90 gamma=90, with a unit cell variability of 5% in all dimensions.

27. A co-crystal of N8 with peramivir having a tetragonal space group I4 having unit cell dimensions a=b=89.78, c=93.23 having a unit cell variability of 5% in all dimensions.


SEQUENCE LISTING (6 sequences)

SEQ ID NO: 1 - protein, 449 aa, Influenza A virus
MNPNQKIITIGSICMVTGIVSLMLQIGNMISIWVSHSIHTGNQHQSEPISNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASYKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 2 - protein, 470 aa, Influenza A virus
MNPNQKIITIGSVSIVLTTIGLLLQITSLCSIWFSHYNQVTQTNEQPCSNNTTNYYNETFVNVTNVQNNYTTITDPSTSQVIHYSSGKDLCPVKGWAPLSKDNGIRIGSRGEVFVIREPFISCSINECRTFFLTQGALLNDKHSNGTVKDRSPFRTLMSCPIGVAPSPSNSRFESVAWSATACSDGPGWLTIGITGPDATAVAVLKYNGIITDTLKSWKGNIMRTQESECVCQDEFCYTLITDGPSDAQAFYKILKIKKGKIVSVKDVDAPGFHFEECSCYPSGENVECVCRDNWRGSNRPWIRFNSDLDYQIGYVCSGVFGDNPRPMDSTGSCNSPINNGKGRYGVKGFSFRYGDGVWIGRTKSLESRSGFEMVWDANGWVSTDKDSNGVQDIIDNDNWSGYSGSFSIRGETTGRNCTVPCFWVEMIRGQPKEKTIWTSGSSIAFCGVNSDTTGWSWPDGALLPFDIDK

SEQ ID NO: 3 - protein, 470 aa, Influenza A virus
MNPNQKIITIGSISLGLVVFNVLLHVVSIIVTVLVLGKGGNNGICNETVVREYNETVRIEKVTQWHNTNVVEYVPYWNGGTYMNNTEAICDAKGFAPFSKDNGIRIGSRGHIFVIREPFVSCSPIECRTFFLTQGSLLNDKHSNGTVKDRSPFRTLMSVEVGQSPNVYQARFEAVAWSATACHDGKKWMTVGVTGPDSKAVAVIHYGGVPTDVVNSWAGDILRTQESSCTCIQGDCYWVMTDGPANRQAQYRIYKANQGRIIGQTDISFNGGHIEECSCYPNDGKVECVCRDGWTGTNRPVLVISPDLSYRVGYLCAGIPSDTPRGEDTQFTGSCTSPMGNQGYGVKGFGFRQGTDVWMGRTISRTSRSGFEILRIKNGWTQTSKEQIRKQVVVDNLNWSGYSGSFTLPVELSGKDCLVPCFWVEMIRGKPEEKTIWTSSSSIVMCGVDYEVADWSWHDGAILPFDIDKM

SEQ ID NO: 4 - protein, 385 aa, Influenza A virus
VKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASYKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTI

SEQ ID NO: 5 - protein, 388 aa, Influenza A virus
VIHYSSGKDLCPVKGWAPLSKDNGIRIGSRGEVFVIREPFISCSINECRTFFLTQGALLNDKHSNGTVKDRSPFRTLMSCPIGVAPSPSNSRFESVAWSATACSDGPGWLTIGITGPDATAVAVLKYNGIITDTLKSWKGNIMRTQESECVCQDEFCYTLITDGPSDAQAFYKILKIKKGKIVSVKDVDAPGFHFEECSCYPSGENVECVCRDNWRGSNRPWIRFNSDLDYQIGYVCSGVFGDNPRPMDSTGSCNSPINNGKGRYGVKGFSFRYGDGVWIGRTKSLESRSGFEMVWDANGWVSTDKDSNGVQDIIDNDNWSGYSGSFSIRGETTGRNCTVPCFWVEMIRGQPKEKTIWTSGSSIAFCGVNSDTTGWSWPDGALLPFDI

SEQ ID NO: 6 - protein, 387 aa, Influenza A virus
TYMNNTEAICDAKGFAPFSKDNGIRIGSRGHIFVIREPFVSCSPIECRTFFLTQGSLLNDKHSNGTVKDRSPFRTLMSVEVGQSPNVYQARFEAVAWSATACHDGKKWMTVGVTGPDSKAVAVIHYGGVPTDVVNSWAGDILRTQESSCTCIQGDCYWVMTDGPANRQAQYRIYKANQGRIIGQTDISFNGGHIEECSCYPNDGKVECVCRDGWTGTNRPVLVISPDLSYRVGYLCAGIPSDTPRGEDTQFTGSCTSPMGNQGYGVKGFGFRQGTDVWMGRTISRTSRSGFEILRIKNGWTQTSKEQIRKQVVVDNLNWSGYSGSFTLPVELSGKDCLVPCFWVEMIRGKPEEKTIWTSSSSIVMCGVDYEVADWSWHDGAILPFDI